SEQUENCE LISTING (277 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 112 aa, Artificial sequence
  Synthesized
EVQLQQSGAELVRPGASVKLSCTASGFNIKDDYMNWVKQRPERGLEWIGWIDPENGDTEYASKFQGKATMTADTSSNTAYLQFSSLTSEDTAVYYCTTSNGWGQGTLVTVST

SEQ ID NO: 8 - protein, 10 aa, Artificial sequence
  Synthesized
GFNIKDDYMN

SEQ ID NO: 9 - protein, 17 aa, Artificial sequence
  Synthesized
WIDPENGDTEYASKFQG

SEQ ID NO: 10 - skipped

SEQ ID NO: 11 - protein, 112 aa, Artificial sequence
  Synthesized
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPLTFGAGTKLELK

SEQ ID NO: 12 - protein, 16 aa, Artificial sequence
  Synthesized
RSSKSLLHSNGITYLY

SEQ ID NO: 13 - protein, 7 aa, Artificial sequence
  Synthesized
QMSNLAS

SEQ ID NO: 14 - protein, 9 aa, Artificial sequence
  Synthesized
AQNLELPLT

SEQ ID NO: 15 - protein, 112 aa, Artificial sequence
  Synthesized
QVQLQQSGAELVKPGASVKLSCTASGFNIKDDYMNWVKQRPEQGLEWIGWIDPENGDTEYASKFQGKATITADTSSNTAYLQLSSLTSEDTAVYYCASSNGWGQGTTLTVSS

SEQ ID NO: 16 - protein, 112 aa, Artificial sequence
  Synthesized
EVQLQQSGAELVKPGATVKISCTASGFNIKDDYMNWVKQRPEQGLEWIGWIDPENGDTEYASKFQGKATMTADTSTNTAYLQLSSLTSEDTAVYYCTTSNGWGQGTTVTVSS

SEQ ID NO: 17 - protein, 112 aa, Artificial sequence
  Synthesized
EVQLQQSGAELVKPGATVKISCTASGFNIKDDYMNWVKQRPEQGLEWIGWIDPENGDTEYASKFQGRATMTADTSTNTAYLELSSLTSEDTAVYYCTTSNGWGQGTTVTVSS

SEQ ID NO: 18 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLELSSLRSEDTAVYYCTTSNGWGQGTTVTVSS

SEQ ID NO: 19 - protein, 112 aa, Artificial sequence
  Synthesized
EVQLQQSGAELVKPGATVKISCKASGFTIKDDYMNWVKQRPEKGLEWIGWVDPEDGETEYASKFQGRATMTADTSTDTAYMELSSLRSEDTAVYYCTTSNGWGQGTLVTVSS

SEQ ID NO: 20 - protein, 112 aa, Artificial sequence
  Synthesized
EVQLQQSGAELVKPGATVKISCKASGFTIKDDYMNWVKQAPEKGLEWMGWVDPEDGETEYASKFQGRATMTADTSTDTAYMELSSLRSEDTAVYYCTTSNGWGQGTLVTVSS

SEQ ID NO: 21 - protein, 112 aa, Artificial sequence
  Synthesized
EVQLVQSGAEVKKPGATVKISCKASGFNIKDDYMNWVRQAPGKGLEWIGWVDPEDGETEYASKFQGRATMTADTSTDTAYMELSSLRSEDTAVYYCTTSNGWGQGTLVTVSS

SEQ ID NO: 22 - protein, 112 aa, Artificial sequence
  Synthesized
EVQLVQSGAEVKKPGATVKISCKASGFNIKDDYMNWVRQAPGKGLEWIGWVDPENGDTEYASKFQGRATMTADTSTDTAYMELSSLRSEDTAVYYCTTSNGWGQGTLVTVSS

SEQ ID NO: 23 - protein, 112 aa, Artificial sequence
  Synthesized
DIVMTQAAFSNPVTLGTSASISCRSSKSLLHSNGITYLYWYLQRPGQSPQLLIYQMSNLASGVPNRFSSSGSGTDFTLRISRVEAEDVGVYYCAQNLELPLTFGGGTKLEIK

SEQ ID NO: 24 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCAQNLELPLTFGQGTKLEIK

SEQ ID NO: 25 - protein, 112 aa, Artificial sequence
  Synthesized
DIVMTQSPFSNPVTPGESASISCRSSKSLLHSNGITYLYWYLQRPGQSPQLLIYQMSNLASGVPNRFSGSGSGTDFTLRISRVEAEDVGVYYCAQNLELPLTFGQGTKLEIK

SEQ ID NO: 26 - protein, 112 aa, Artificial sequence
  Synthesized
DIVMTQSPFSLPVTPGESASISCRSSKSLLHSNGITYLYWYLQRPGQSPQLLIYQGSNRASGVPNRFSGSGSGTDFTLRISRVEAEDVGVYYCAQNLELPLTFGQGTKLEIK

SEQ ID NO: 27 - protein, 112 aa, Artificial sequence
  Synthesized
DIVMTQSPFSLPVTPGESASISCRSSKSLLHSNGYTYLYWYLQRPGQSPQLLIYQGSNRASGVPNRFSGSGSGTDFTLRISRVEAEDVGVYYCAQNLELPLTFGQGTKLEIK

SEQ ID NO: 28 - protein, 112 aa, Artificial sequence
  Synthesized
DIVMTQSPFSLPVTPGESASISCRSSKSLLHSNGYTYLYWYLQRPGQSPQLLIYQGSNRASGVPNRFSGSESGTDFTLRISRVEAEDVGVYYCAQNLELPLTFGQGTKLEIK

SEQ ID NO: 29 - protein, 112 aa, Artificial sequence
  Synthesized
DIVMTQSPLSLPVTPGEPASISCRSSKSLLHSNGINYLYWYLQKPGQSPQLLIYQGSNRASGVPDRFSGSGSGTDFTLKISRVEAEDVGVYYCAQNLELPLTFGQGTKLEIK

SEQ ID NO: 30 - protein, 112 aa, Artificial sequence
  Synthesized
EVQLQQSGAELVEPGASVKLSCTGSGFNIKVYYVHWLKQLTEQGLEWIGRIDPENGETIYTPKFQDKATLTVDTSSNTAYLQLSSLTSEDAAVYYCVSSGYWGQGTTLTVSS

SEQ ID NO: 31 - protein, 119 aa, Homo sapiens
EVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAGYRSPMPTANKWGQGTLVTVSS

SEQ ID NO: 32 - protein, 114 aa, Homo sapiens
QVQLQQSGAELVKPGASVKLSCTASGFNIKDTYMHWVKQRPEQGLEWIGRIDPANGNTKYDPKFQGKATITADTSSNTAYLQLSSLTSEDTAVYYCASYYGIYWGQGTTLTVSS

SEQ ID NO: 33 - protein, 111 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATQHWGQGTLVTVSS

SEQ ID NO: 34 - protein, 111 aa, Artificial sequence
  Synthesized
DIVMTQSAFSNPVTLGTSASISCRSSKSLLHRNGITYLYWYLQKPGQPPQLLIYQMSNLASGVPDRFTSSGSGTDFTLKISRVEAEDVGVYYCAQNLELWTFGGGTKLEIK

SEQ ID NO: 35 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 36 - protein, 112 aa, Homo sapiens [sequence omitted]

SEQ ID NO: 37 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 38 - DNA, 393 bp, Artificial sequence
  Synthesized
atgaaatgcagctgggtcatcttcttcctgatggcagtggttataggggtcaattcagaggttcagctgcagcagtctggggctgaacttgtgaggccaggggcctcagtcaagttgtcctgcacagcttctggctttaacattaaagacgactatatgaactgggtgaaacagagacctgaacggggcctggagtggattggatggattgatcctgagaatggtgatactgaatatgcctcgaagttccagggaaaggccactatgactgcagacacatcctccaacacagcctacctgcagttcagcagcctgacatctgaggacactgccgtctattactgtactacaagtaacggctggggccaagggactctggtcactgtctctaca

SEQ ID NO: 39 - DNA, 396 bp, Artificial sequence
  Synthesized
atgaggttctctgctcagcttctggggctgcttgtgctctggatccctggatccattgcagatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgctcacgttcggtgctgggaccaagctggagctgaaa

SEQ ID NO: 40 - protein, 5 aa, Artificial sequence
  Synthesized
DDYMN

SEQ ID NO: 41 - protein, 7 aa, Artificial sequence
  Synthesized
GFNIKDD

SEQ ID NO: 42 - protein, 6 aa, Artificial sequence
  Synthesized
DPENGD

SEQ ID NO: 43 - protein, 10 aa, Artificial sequence
  Synthesized
WIDPENGDTE

SEQ ID NO: 44 - protein, 6 aa, Artificial sequence
  Synthesized
KDDYMN

SEQ ID NO: 45 - protein, 13 aa, Artificial sequence
  Synthesized
WIGWIDPENGDTE

SEQ ID NO: 46 - protein, 4 aa, Artificial sequence
  Synthesized
TTSN

SEQ ID NO: 47 - protein, 7 aa, Artificial sequence
  Synthesized
ITYLYWY

SEQ ID NO: 48 - protein, 10 aa, Artificial sequence
  Synthesized
LLIYQMSNLA

SEQ ID NO: 49 - protein, 8 aa, Artificial sequence
  Synthesized
AQNLELPL

SEQ ID NO: 50 - protein, 10 aa, Artificial sequence
  Synthesized
GFTIKDDYMN

SEQ ID NO: 51 - protein, 17 aa, Artificial sequence
  Synthesized
WVDPEDGETEYASKFQG

SEQ ID NO: 52 - protein, 17 aa, Artificial sequence
  Synthesized
WVDPENGDTEYASKFQG

SEQ ID NO: 53 - protein, 16 aa, Artificial sequence
  Synthesized
RSSKSLLHSNGYTYLY

SEQ ID NO: 54 - protein, 16 aa, Artificial sequence
  Synthesized
RSSKSLLHSNGINYLY

SEQ ID NO: 55 - protein, 7 aa, Artificial sequence
  Synthesized
QGSNRAS

SEQ ID NO: 56 - protein, 6 aa, Artificial sequence
  Synthesized
  MISC_FEATURE (1)..(1): Xaa = Gln or Glu
  MISC_FEATURE (6)..(6): Xaa = Ser or Pro
XIVYKX

SEQ ID NO: 57 - protein, 6 aa, Artificial sequence
  Synthesized
QIVYKP

SEQ ID NO: 58 - protein, 7 aa, Artificial sequence
  Synthesized
EIVYKSP

SEQ ID NO: 59 - protein, 7 aa, Artificial sequence
  Synthesized
GSGSGSG

SEQ ID NO: 60 - protein, 10 aa, Artificial sequence
  Synthesized
YPYDVPDYAG

SEQ ID NO: 61 - protein, 112 aa, Artificial sequence
  Synthesized
DIVMTQSPFSNPVTPGTSASISCRSSKSLLHSNGITYLYWYLQRPGQSPQLLIYQESNLASGVPNRFSSSGSGTDFTLRISRVEAEDVGVYYCAQNLELPLTFGQGTKLEIK

SEQ ID NO: 62 - protein, 112 aa, Artificial sequence
  Synthesized
DIVMTQSPFSNPVTPGTSASISCRSSKSLLHSNGITYLYWYLQRPGQSPQLLIYQDSNLASGVPNRFSSSGSGTDFTLRISRVEAEDVGVYYCAQNLELPLTFGQGTKLEIK

SEQ ID NO: 63 - protein, 112 aa, Artificial sequence
  Synthesized
DIVMTQSPFSNPVTPGTSASISCRSSKSLDHSNGITYLYWYLQRPGQSPQLLIYQMSNLASGVPNRFSSSGSGTDFTLRISRVEAEDVGVYYCAQNLELPLTFGQGTKLEIK

SEQ ID NO: 64 - protein, 112 aa, Artificial sequence
  Synthesized
DIVMTQSPFSNPVTPGTSASISCRSSKSLGHSNGITYLYWYLQRPGQSPQLLIYQMSNLASGVPNRFSSSGSGTDFTLRISRVEAEDVGVYYCAQNLELPLTFGQGTKLEIK

SEQ ID NO: 65 - protein, 112 aa, Artificial sequence
  Synthesized
DIVMTQSPFSNPVTPGTSASISCRSSKSLSHSNGITYLYWYLQRPGQSPQLLIYQMSNLASGVPNRFSSSGSGTDFTLRISRVEAEDVGVYYCAQNLELPLTFGQGTKLEIK

SEQ ID NO: 66 - protein, 112 aa, Artificial sequence
  Synthesized
DIVMTQSPFSNPVTPGTSASISCRSSKSLEHSNGITYLYWYLQRPGQSPQLLIYQMSNLASGVPNRFSSSGSGTDFTLRISRVEAEDVGVYYCAQNLELPLTFGQGTKLEIK

SEQ ID NO: 67 - protein, 112 aa, Artificial sequence
  Synthesized
DIVMTQSPFSNPVTPGTSASISCRSSKSLLHSNGETYLYWYLQRPGQSPQLLIYQMSNLASGVPNRFSSSGSGTDFTLRISRVEAEDVGVYYCAQNLELPLTFGQGTKLEIK

SEQ ID NO: 68 - protein, 112 aa, Artificial sequence
  Synthesized
DIVMTQSPFSNPVTPGTSASISCRSSKSLLHSNGKTYLYWYLQRPGQSPQLLIYQMSNLASGVPNRFSSSGSGTDFTLRISRVEAEDVGVYYCAQNLELPLTFGQGTKLEIK

SEQ ID NO: 69 - protein, 112 aa, Artificial sequence
  Synthesized
DIVMTQSPFSNPVTPGTSASISCRSSKSLTHSNGITYLYWYLQRPGQSPQLLIYQMSNLASGVPNRFSSSGSGTDFTLRISRVEAEDVGVYYCAQNLELPLTFGQGTKLEIK

SEQ ID NO: 70 - protein, 112 aa, Artificial sequence
  Synthesized
DIVMTQSPFSNPVTPGTSASISCRSSKSLNHSNGITYLYWYLQRPGQSPQLLIYQMSNLASGVPNRFSSSGSGTDFTLRISRVEAEDVGVYYCAQNLELPLTFGQGTKLEIK

SEQ ID NO: 71 - protein, 112 aa, Artificial sequence
  Synthesized
DIVMTQSPFSNPVTPGTSASISCRSSKSDLHSNGITYLYWYLQRPGQSPQLLIYQMSNLASGVPNRFSSSGSGTDFTLRISRVEAEDVGVYYCAQNLELPLTFGQGTKLEIK

SEQ ID NO: 72 - protein, 112 aa, Artificial sequence
  Synthesized
DIVMTQSPFSNPVTPGTSASISCRSSKSLLHSNGGTYLYWYLQRPGQSPQLLIYQMSNLASGVPNRFSSSGSGTDFTLRISRVEAEDVGVYYCAQNLELPLTFGQGTKLEIK

SEQ ID NO: 73 - protein, 112 aa, Artificial sequence
  Synthesized
DIVMTQSPFSNPVTPGTSASISCRSSKSLLHSNGITYNYWYLQRPGQSPQLLIYQMSNLASGVPNRFSSSGSGTDFTLRISRVEAEDVGVYYCAQNLELPLTFGQGTKLEIK

SEQ ID NO: 74 - protein, 112 aa, Artificial sequence
  Synthesized
DIVMTQSPFSNPVTPGTSASISCRSSKSLAHSNGITYLYWYLQRPGQSPQLLIYQMSNLASGVPNRFSSSGSGTDFTLRISRVEAEDVGVYYCAQNLELPLTFGQGTKLEIK

SEQ ID NO: 75 - protein, 112 aa, Artificial sequence
  Synthesized
DIVMTQSPFSNPVTPGTSASISCRSSKSLLHSNGITYTYWYLQRPGQSPQLLIYQMSNLASGVPNRFSSSGSGTDFTLRISRVEAEDVGVYYCAQNLELPLTFGQGTKLEIK

SEQ ID NO: 76 - protein, 112 aa, Artificial sequence
  Synthesized
DIVMTQSPFSNPVTPGTSASISCRSSKSLLHSNGITYSYWYLQRPGQSPQLLIYQMSNLASGVPNRFSSSGSGTDFTLRISRVEAEDVGVYYCAQNLELPLTFGQGTKLEIK

SEQ ID NO: 77 - protein, 112 aa, Artificial sequence
  Synthesized
DIVMTQSPFSNPVTPGTSASISCRSSKSLLHSNGITYRYWYLQRPGQSPQLLIYQMSNLASGVPNRFSSSGSGTDFTLRISRVEAEDVGVYYCAQNLELPLTFGQGTKLEIK

SEQ ID NO: 78 - protein, 112 aa, Artificial sequence
  Synthesized
DIVMTQSPFSNPVTPGTSASISCRSSKSLLHSNGQTYLYWYLQRPGQSPQLLIYQMSNLASGVPNRFSSSGSGTDFTLRISRVEAEDVGVYYCAQNLELPLTFGQGTKLEIK

SEQ ID NO: 79 - protein, 112 aa, Artificial sequence
  Synthesized
DIVMTQSPFSNPVTPGTSASISCRSSKSTLHSNGITYLYWYLQRPGQSPQLLIYQMSNLASGVPNRFSSSGSGTDFTLRISRVEAEDVGVYYCAQNLELPLTFGQGTKLEIK

SEQ ID NO: 80 - protein, 112 aa, Artificial sequence
  Synthesized
DIVMTQSPFSNPVTPGTSASISCRSSKSLLHSNGIGYLYWYLQRPGQSPQLLIYQMSNLASGVPNRFSSSGSGTDFTLRISRVEAEDVGVYYCAQNLELPLTFGQGTKLEIK

SEQ ID NO: 81 - protein, 112 aa, Artificial sequence
  Synthesized
DIVMTQSPFSNPVTPGTSASISCRSSKSQLHSNGITYLYWYLQRPGQSPQLLIYQMSNLASGVPNRFSSSGSGTDFTLRISRVEAEDVGVYYCAQNLELPLTFGQGTKLEIK

SEQ ID NO: 82 - protein, 112 aa, Artificial sequence
  Synthesized
DIVMTQSPFSNPVTPGTSASISCRSSKSLLHSNGITYGYWYLQRPGQSPQLLIYQMSNLASGVPNRFSSSGSGTDFTLRISRVEAEDVGVYYCAQNLELPLTFGQGTKLEIK

SEQ ID NO: 83 - protein, 112 aa, Artificial sequence
  Synthesized
DIVMTQSPFSNPVTPGTSASISCRSSKSLPHSNGITYLYWYLQRPGQSPQLLIYQMSNLASGVPNRFSSSGSGTDFTLRISRVEAEDVGVYYCAQNLELPLTFGQGTKLEIK

SEQ ID NO: 84 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRREAEDVGVYYCAQNLELPLTFGQGTKLEIK

SEQ ID NO: 85 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRDSRVEAEDVGVYYCAQNLELPLTFGQGTKLEIK

SEQ ID NO: 86 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRDEAEDVGVYYCAQNLELPLTFGQGTKLEIK

SEQ ID NO: 87 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISREEAEDVGVYYCAQNLELPLTFGQGTKLEIK

SEQ ID NO: 88 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRPEAEDVGVYYCAQNLELPLTFGQGTKLEIK

SEQ ID NO: 89 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRKEAEDVGVYYCAQNLELPLTFGQGTKLEIK

SEQ ID NO: 90 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISDVEAEDVGVYYCAQNLELPLTFGQGTKLEIK

SEQ ID NO: 91 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRGEAEDVGVYYCAQNLELPLTFGQGTKLEIK

SEQ ID NO: 92 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRIPRVEAEDVGVYYCAQNLELPLTFGQGTKLEIK

SEQ ID NO: 93 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRPSRVEAEDVGVYYCAQNLELPLTFGQGTKLEIK

SEQ ID NO: 94 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRQSRVEAEDVGVYYCAQNLELPLTFGQGTKLEIK

SEQ ID NO: 95 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRGSRVEAEDVGVYYCAQNLELPLTFGQGTKLEIK

SEQ ID NO: 96 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTPRISRVEAEDVGVYYCAQNLELPLTFGQGTKLEIK

SEQ ID NO: 97 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTGRISRVEAEDVGVYYCAQNLELPLTFGQGTKLEIK

SEQ ID NO: 98 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRQEAEDVGVYYCAQNLELPLTFGQGTKLEIK

SEQ ID NO: 99 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRIGRVEAEDVGVYYCAQNLELPLTFGQGTKLEIK

SEQ ID NO: 100 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCAQNDELPLTFGQGTKLEIK

SEQ ID NO: 101 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVTYCAQNLELPLTFGQGTKLEIK

SEQ ID NO: 102 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCAQNEELPLTFGQGTKLEIK

SEQ ID NO: 103 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCAQNGELPLTFGQGTKLEIK

SEQ ID NO: 104 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCAQNQELPLTFGQGTKLEIK

SEQ ID NO: 105 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCAQNLEGPLTFGQGTKLEIK

SEQ ID NO: 106 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGGYYCAQNLELPLTFGQGTKLEIK

SEQ ID NO: 107 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCAQNTELPLTFGQGTKLEIK

SEQ ID NO: 108 - protein, 112 aa, Artificial sequence
  Synthesized
DIVMTQSPFSNPVTPGTSASISCRSSKSLLHSNGITYLYWYLQRPGQSPQLLIYQMSNLASGVPNRFSSSGSGTDFTLRISRVEAEDVGVYYCGQNLELPLTFGQGTKLEIK

SEQ ID NO: 109 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYPELSSLRSEDTAVYYCTTSNGWGQGTTVTVSS

SEQ ID NO: 110 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYDELSSLRSEDTAVYYCTTSNGWGQGTTVTVSS

SEQ ID NO: 111 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLELSSGRSEDTAVYYCTTSNGWGQGTTVTVSS

SEQ ID NO: 112 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLELSSDRSEDTAVYYCTTSNGWGQGTTVTVSS

SEQ ID NO: 113 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLEPSSLRSEDTAVYYCTTSNGWGQGTTVTVSS

SEQ ID NO: 114 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYGELSSLRSEDTAVYYCTTSNGWGQGTTVTVSS

SEQ ID NO: 115 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLEKSSLRSEDTAVYYCTTSNGWGQGTTVTVSS

SEQ ID NO: 116 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLERSSLRSEDTAVYYCTTSNGWGQGTTVTVSS

SEQ ID NO: 117 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLEESSLRSEDTAVYYCTTSNGWGQGTTVTVSS

SEQ ID NO: 118 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLENSSLRSEDTAVYYCTTSNGWGQGTTVTVSS

SEQ ID NO: 119 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTADLELSSLRSEDTAVYYCTTSNGWGQGTTVTVSS

SEQ ID NO: 120 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTANLELSSLRSEDTAVYYCTTSNGWGQGTTVTVSS

SEQ ID NO: 121 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAGLELSSLRSEDTAVYYCTTSNGWGQGTTVTVSS

SEQ ID NO: 122 - protein, 112 aa, Artificial sequence
  Synthesized
EVQLQQSGAELVKPGATVKISCKASGFTIKDDYMNWVKQRPEKGLEWIGWVDPEDGETEYASKFQGRATMTADTSTDTAYEELSSLRSEDTAVYYCTTSNGWGQGTLVTVSS

SEQ ID NO: 123 - protein, 112 aa, Artificial sequence
  Synthesized
EVQLQQSGAELVKPGATVKISCKASGFTIKDDYMNWVKQRPEKGLEWIGWVDPEDGETEYASKFQGRATMTADTSTDTAYGELSSLRSEDTAVYYCTTSNGWGQGTLVTVSS

SEQ ID NO: 124 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLELSSSRSEDTAVYYCTTSNGWGQGTTVTVSS

SEQ ID NO: 125 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAQLELSSLRSEDTAVYYCTTSNGWGQGTTVTVSS

SEQ ID NO: 126 - protein, 112 aa, Artificial sequence
  Synthesized
EVQLQQSGAELVKPGATVKISCKASGFTIKDDYMNWVKQRPEQGLEWIGWIDPENGDTEYASKFQGRATMTADTSTNTAYLELGSLRSEDTAVYYCTTSNGWGQGTTVTVSS

SEQ ID NO: 127 - protein, 112 aa, Artificial sequence
  Synthesized
EVQLVQSGAEVKKPGATVKISCKASGFNIKDDYMNWVQQRPGKGLEWIGWIDPENGDTEYASKFQGRATMTADTSTNTAYMELSSLRSEDTAVYYCTTSNGWGQGTLVTVSS

SEQ ID NO: 128 - protein, 112 aa, Artificial sequence
  Synthesized
EVQLVQSGAEVKKPGATVKISCKASGFNIKDDYMNWVKQRPEKGLEWIGWIDPENGDTEYASKFQGRATMTADTSTNTAYMELSSLRSEDTAVYYCTTSNGWGQGTLVTVSS

SEQ ID NO: 129 - protein, 112 aa, Artificial sequence
  Synthesized
EVQLVQSGAEVKKPGATVKISCKASGFNIKDDYMNWVKQRPEKGLEWIGWIDPENGDTEYASKFQGRATMTADTSTNTAYMELSSGRSEDTAVYYCTTSNGWGQGTLVTVSS

SEQ ID NO: 130 - protein, 112 aa, Artificial sequence
  Synthesized
DIVMTQSPFSLPVTPGESASISCRSSKSLLHSNGITYLYWYLQKPGQSPQLLIYQMSNLASGVPNRFSSSGSGTDFTLKISRVEAEDVGVYYCAQNLELPLTFGQGTKLEIK

SEQ ID NO: 131 - protein, 112 aa, Artificial sequence
  Synthesized
DIVMTQSPFSLPVTPGESASISCRSSKSLLHSNGITYLYWYLQKPGQSPQLLIYQMSNLASGVPDRFSSSGSGTDFTLKISRVEAEDVGVYYCAQNLELPLTFGQGTKLEIK

SEQ ID NO: 132 - protein, 112 aa, Artificial sequence
  Synthesized
DIQMTQSPFSLPVTPGESASISCRSSKSLSHSNGITYLYWYQQKPGQSPQLLIYQGSNGASGVPNRFSSSGSGTDFTLKISRVEAEDVGVYYCAQNIELPLTFGQGTKLEIK

SEQ ID NO: 133 - protein, 112 aa, Artificial sequence
  Synthesized
DIQMTQSPFSLPVTPGESASISCRSSKSLSHSNGITYLYWYQQKPGQSPQLLIYQGSNRASGVPNRFSSSGSGTDFTLKISRVEAEDVGVYYCAQNIELPLTFGQGTKLEIK

SEQ ID NO: 134 - protein, 112 aa, Artificial sequence
  Synthesized
DIQMTQSPFSLPVTPGESASISCRSSKSLSHSNGITYLYWYQQKPGQSPQLLIYQGSNTASGVPNRFSSSGSGTDFTLKISRVEAEDVGVYYCAQNIELPLTFGQGTKLEIK

SEQ ID NO: 135 - protein, 112 aa, Artificial sequence
  Synthesized
DIQMTQSPFSLPVTPGESASISCRSSKSLSHSNGITYLYWYQQKPGQSPQLLIYQGSNRASGVPNRFSSSGSGTDFTLKISRVEAEDVGVYYCAQNGELPLTFGQGTKLEIK

SEQ ID NO: 136 - protein, 112 aa, Artificial sequence
  Synthesized
DIQMTQSPFSLPVTPGESASISCRSSKSLGHSNGITYLYWYQQKPGQSPQLLIYQGSNRASGVPNRFSSSGSGTDFTLKISRVEAEDVGVYYCAQNIELPLTFGQGTKLEIK

SEQ ID NO: 137 - protein, 112 aa, Artificial sequence
  Synthesized
DIQMTQSPFSLPVTPGESASISCRSSKSLDHSNGITYLYWYQQKPGQSPQLLIYQGSNRASGVPNRFSSSGSGTDFTLKISRVEAEDVGVYYCAQNIELPLTFGQGTKLEIK

SEQ ID NO: 138 - protein, 112 aa, Artificial sequence
  Synthesized
DIQMTQSPFSLPVTPGESASISCRSSKSLDHSNGITYLYWYQQKPGQSPQLLIYQKSNRASGVPNRFSSSGSGTDFTLKISRVEAEDVGVYYCAQNIELPLTFGQGTKLEIK

SEQ ID NO: 139 - protein, 112 aa, Artificial sequence
  Synthesized
DIQMTQSPFSLPVTPGESASISCRSSKSLGHSNGITYLYWYQQKPGQSPQLLIYQKSNRASGVPNRFSSSGSGTDFTLKISRVEAEDVGVYYCAQNIELPLTFGQGTKLEIK

SEQ ID NO: 140 - protein, 112 aa, Artificial sequence
  Synthesized
DIQMTQSPFSLPVTPGESASISCRSSKSLGHSNGITYLYWYQQKPGQSPQLLIYQKSNGASGVPNRFSSSGSGTDFTLKISRVEAEDVGVYYCAQNIELPLTFGQGTKLEIK

SEQ ID NO: 141 - protein, 112 aa, Artificial sequence
  Synthesized
DIQMTQSPFSLPVTPGESASISCRSSKSLSHSNGITYLYWYQQKPGQSPQLLIYQKSNGASGVPNRFSSSGSGTDFTLKISRVEAEDVGVYYCAQNIELPLTFGQGTKLEIK

SEQ ID NO: 142 - protein, 112 aa, Artificial sequence
  Synthesized
DIQMTQSPFSLPVTPGESASISCRSSKSLGHSNGITYLYWYGQKPGQSPQLLIYQGSNRASGVPNRFSSSGSGTDFTLKISRVEAEDVGVYYCAQNIELPLTFGQGTKLEIK

SEQ ID NO: 143 - protein, 112 aa, Artificial sequence
  Synthesized
DIQMTQSPFSLPVTPGESASISCRSSKSLGHSNGITYLYWYGQKPGQSPQLLIYQGSNRASGVPNRFSSSGSGTDFTLKISRVEAEDVGVYYCAQNGELPLTFGQGTKLEIK

SEQ ID NO: 144 - protein, 112 aa, Artificial sequence
  Synthesized
DIQMTQSPFSLPVTPGESASISCRSSKSLGHSNGITYLYWYGQKPGQSPQLLIYQGSNRASGVPNRFSSSGSGTDFTLKISRVEAEDVGVYYCAQNLELPLTFGQGTKLEIK

SEQ ID NO: 145 - protein, 112 aa, Artificial sequence
  Synthesized
DIQMTQSPFSLPVTPGESASISCRSSKSLGHSNGITYLYWYGQKPGQSPQLLIYQGSNTASGVPNRFSSSGSGTDFTLKISRVEAEDVGVYYCAQNIELPLTFGQGTKLEIK

SEQ ID NO: 146 - protein, 112 aa, Artificial sequence
  Synthesized
DIQMTQSPFSLPVTPGESASISCRSSKSLGHSNGITYLYWYGQKPGQSPQLLIYQGSNTASGVPNRFSSSGSGTDFTLKISRVEAEDVGVYYCAQNGELPLTFGQGTKLEIK

SEQ ID NO: 147 - protein, 112 aa, Artificial sequence
  Synthesized
DIQMTQSPFSLPVTPGESASISCRSSKSLGHSNGITYLYWYGQKPGQSPQLLIYQGSNTASGVPNRFSSSGSGTDFTLKISRVEAEDVGVYYCAQNLELPLTFGQGTKLEIK

SEQ ID NO: 148 - protein, 112 aa, Artificial sequence
  Synthesized
DIQMTQSPFSLPVTPGESASISCRSSKSLSHSNGITYLYWYGQKPGQSPQLLIYQGSNTASGVPNRFSSSGSGTDFTLKISRVEAEDVGVYYCAQNIELPLTFGQGTKLEIK

SEQ ID NO: 149 - protein, 112 aa, Artificial sequence
  Synthesized
DIQMTQSPFSLPVTPGESASISCRSSKSLDHSNGITYLYWYGQKPGQSPQLLIYQGSNRASGVPNRFSSSGSGTDFTLKISRVEAEDVGVYYCAQNIELPLTFGQGTKLEIK

SEQ ID NO: 150 - protein, 112 aa, Artificial sequence
  Synthesized
DIQMTQSPFSLPVTPGESASISCRSSKSLSHSNGITYLYWYIQKPGQSPQLLIYQISNRASGVPNRFSSSGSGTDFTLKISRVEAEDVGVYYCAQNIELPLTFGQGTKLEIK

SEQ ID NO: 151 - protein, 112 aa, Artificial sequence
  Synthesized
DIQMTQSPFSLPVTPGESASISCRSSKSLSHSNGITYLYWYQQKPGQSPQLLIYQISNGASGVPNRFSSSGSGTDFTLKISRVEAEDVGVYYCAQNIELPLTFGQGTKLEIK

SEQ ID NO: 152 - protein, 112 aa, Artificial sequence
  Synthesized
DIQMTQSPFSLPVTPGESASISCRSSKSLSHSNGITYLYWYQQKPGQSPQLLIYQISNGASGVPNRFSSSGSGTDFTLKISRVEAEDVGVYYCAQNLELPLTFGQGTKLEIK

SEQ ID NO: 153 - protein, 112 aa, Artificial sequence
  Synthesized
DIQMTQSPFSLPVTPGESASISCRSSKSLSHSNGITYLYWYQQKPGQSPQLLIYQESNRASGVPNRFSSSGSGTDFTLKISRVEAEDVGVYYCAQNIELPLTFGQGTKLEIK

SEQ ID NO: 154 - protein, 112 aa, Artificial sequence
  Synthesized
DIQMTQSPFSLPVTPGESASISCRSSKSLGHSNGITYLYWYQQKPGQSPQLLIYQESNGASGVPNRFSSSGSGTDFTLKISRVEAEDVGVYYCAQNIELPLTFGQGTKLEIK

SEQ ID NO: 155 - protein, 112 aa, Artificial sequence
  Synthesized
DIQMTQSPFSLPVTPGESASISCRSSKSLGHSNGITYLYWYIQKPGQSPQLLIYQESNRASGVPNRFSSSGSGTDFTLKISRVEAEDVGVYYCAQNIELPLTFGQGTKLEIK

SEQ ID NO: 156 - protein, 112 aa, Artificial sequence
  Synthesized
DIQMTQSPFSLPVTPGESASISCRSSKSLGHSNGITYLYWYIQKPGQSPQLLIYQESNRASGVPNRFSSSGSGTDFTLKISRVEAEDVGVYYCAQNGELPLTFGQGTKLEIK

SEQ ID NO: 157 - protein, 112 aa, Artificial sequence
  Synthesized
DIQMTQSPFSLPVTPGESASISCRSSKSLIHSNGITYLYWYIQKPGQSPQLLIYQESNRASGVPNRFSSSGSGTDFTLKISRVEAEDVGVYYCAQNLELPLTFGQGTKLEIK

SEQ ID NO: 158 - protein, 112 aa, Artificial sequence
  Synthesized
DIQMTQSPFSLPVTPGESASISCRSSKSLLHSNGITYLYWYQQKPGQSPQLLIYQGSNRASGVPNRFSSSGSGTDFTLKISRVEAEDVGVYYCAQNIELPLTFGQGTKLEIK

SEQ ID NO: 159 - protein, 112 aa, Artificial sequence
  Synthesized
DIQMTQSPFSLPVTPGESASISCRSSKSLSHSNGITYLYWYLQKPGQSPQLLIYQGSNRASGVPNRFSSSGSGTDFTLKISRVEAEDVGVYYCAQNIELPLTFGQGTKLEIK

SEQ ID NO: 160 - protein, 112 aa, Artificial sequence
  Synthesized
DIQMTQSPFSLPVTPGESASISCRSSKSLSHSNGITYLYWYQQKPGQSPQLLIYQMSNRASGVPNRFSSSGSGTDFTLKISRVEAEDVGVYYCAQNIELPLTFGQGTKLEIK

SEQ ID NO: 161 - protein, 112 aa, Artificial sequence
  Synthesized
DIQMTQSPFSLPVTPGESASISCRSSKSLSHSNGITYLYWYQQKPGQSPQLLIYQGSNLASGVPNRFSSSGSGTDFTLKISRVEAEDVGVYYCAQNIELPLTFGQGTKLEIK

SEQ ID NO: 162 - protein, 112 aa, Artificial sequence
  Synthesized
DIQMTQSPFSLPVTPGESASISCRSSKSLSHSNGITYLYWYQQKPGQSPQLLIYQGSNGASGVPDRFSSSGSGTDFTLKISRVEAEDVGVYYCAQNIELPLTFGQGTKLEIK

SEQ ID NO: 163 - protein, 112 aa, Artificial sequence
  Synthesized
DIQMTQSPFSLPVTPGESASISCRSSKSLSHSNGITYLYWYQQKPGQSPQLLIYQGSNRASGVPDRFSSSGSGTDFTLKISRVEAEDVGVYYCAQNIELPLTFGQGTKLEIK

SEQ ID NO: 164 - protein, 112 aa, Artificial sequence
  Synthesized
DIQMTQSPFSLPVTPGESASISCRSSKSLSHSNGITYLYWYQQKPGQSPQLLIYQGSNRASGVPDRFSSSGSGTDFTLKISRVEAEDVGVYYCAQNGELPLTFGQGTKLEIK

SEQ ID NO: 165 - protein, 112 aa, Artificial sequence
  Synthesized
DIQMTQSPFSLPVTPGESASISCRSSKSLGHSNGITYLYWYQQKPGQSPQLLIYQGSNRASGVPDRFSSSGSGTDFTLKISRVEAEDVGVYYCAQNIELPLTFGQGTKLEIK

SEQ ID NO: 166 - protein, 112 aa, Artificial sequence
  Synthesized
DIQMTQSPFSLPVTPGESASISCRSSKSLGHSNGITYLYWYQQKPGQSPQLLIYQKSNRASGVPDRFSSSGSGTDFTLKISRVEAEDVGVYYCAQNIELPLTFGQGTKLEIK

SEQ ID NO: 167 - protein, 112 aa, Artificial sequence
  Synthesized
DIQMTQSPFSLPVTPGESASISCRSSKSLSHSNGITYLYWYQQKPGQSPQLLIYQKSNGASGVPDRFSSSGSGTDFTLKISRVEAEDVGVYYCAQNIELPLTFGQGTKLEIK

SEQ ID NO: 168 - protein, 112 aa, Artificial sequence
  Synthesized
DIQMTQSPFSLPVTPGESASISCRSSKSLGHSNGITYLYWYGQKPGQSPQLLIYQGSNRASGVPDRFSSSGSGTDFTLKISRVEAEDVGVYYCAQNIELPLTFGQGTKLEIK

SEQ ID NO: 169 - protein, 112 aa, Artificial sequence
  Synthesized
DIQMTQSPFSLPVTPGESASISCRSSKSLGHSNGITYLYWYGQKPGQSPQLLIYQGSNRASGVPDRFSSSGSGTDFTLKISRVEAEDVGVYYCAQNLELPLTFGQGTKLEIK

SEQ ID NO: 170 - protein, 112 aa, Artificial sequence
  Synthesized
DIQMTQSPFSLPVTPGESASISCRSSKSLSHSNGITYLYWYIQKPGQSPQLLIYQISNRASGVPDRFSSSGSGTDFTLKISRVEAEDVGVYYCAQNIELPLTFGQGTKLEIK

SEQ ID NO: 171 - protein, 112 aa, Artificial sequence
  Synthesized
DIQMTQSPFSLPVTPGESASISCRSSKSLSHSNGITYLYWYQQKPGQSPQLLIYQISNGASGVPDRFSSSGSGTDFTLKISRVEAEDVGVYYCAQNIELPLTFGQGTKLEIK

SEQ ID NO: 172 - protein, 16 aa, Artificial sequence
  Synthesized
RSSKSDLHSNGITYLY

SEQ ID NO: 173 - protein, 16 aa, Artificial sequence
  Synthesized
RSSKSTLHSNGITYLY

SEQ ID NO: 174 - protein, 16 aa, Artificial sequence
  Synthesized
RSSKSQLHSNGITYLY

SEQ ID NO: 175 - protein, 16 aa, Artificial sequence
  Synthesized
RSSKSLDHSNGITYLY

SEQ ID NO: 176 - protein, 16 aa, Artificial sequence
  Synthesized
RSSKSLGHSNGITYLY

SEQ ID NO: 177 - protein, 16 aa, Artificial sequence
  Synthesized
RSSKSLSHSNGITYLY

SEQ ID NO: 178 - protein, 16 aa, Artificial sequence
  Synthesized
RSSKSLEHSNGITYLY

SEQ ID NO: 179 - protein, 16 aa, Artificial sequence
  Synthesized
RSSKSLTHSNGITYLY

SEQ ID NO: 180 - protein, 16 aa, Artificial sequence
  Synthesized
RSSKSLNHSNGITYLY

SEQ ID NO: 181 - protein, 16 aa, Artificial sequence
  Synthesized
RSSKSLAHSNGITYLY

SEQ ID NO: 182 - protein, 16 aa, Artificial sequence
  Synthesized
RSSKSLPHSNGITYLY

SEQ ID NO: 183 - protein, 16 aa, Artificial sequence
  Synthesized
RSSKSLIHSNGITYLY

SEQ ID NO: 184 - protein, 16 aa, Artificial sequence
  Synthesized
RSSKSLLHSNGETYLY

SEQ ID NO: 185 - protein, 16 aa, Artificial sequence
  Synthesized
RSSKSLLHSNGKTYLY

SEQ ID NO: 186 - protein, 16 aa, Artificial sequence
  Synthesized
RSSKSLLHSNGGTYLY

SEQ ID NO: 187 - protein, 16 aa, Artificial sequence
  Synthesized
RSSKSLLHSNGQTYLY

SEQ ID NO: 188 - protein, 16 aa, Artificial sequence
  Synthesized
RSSKSLLHSNGIGYLY

SEQ ID NO: 189 - protein, 16 aa, Artificial sequence
  Synthesized
RSSKSLLHSNGITYNY

SEQ ID NO: 190 - protein, 16 aa, Artificial sequence
  Synthesized
RSSKSLLHSNGITYTY

SEQ ID NO: 191 - protein, 16 aa, Artificial sequence
  Synthesized
RSSKSLLHSNGITYSY

SEQ ID NO: 192 - protein, 16 aa, Artificial sequence
  Synthesized
RSSKSLLHSNGITYRY

SEQ ID NO: 193 - protein, 16 aa, Artificial sequence
  Synthesized
RSSKSLLHSNGITYGY

SEQ ID NO: 194 - protein, 7 aa, Artificial sequence
  Synthesized
QESNLAS

SEQ ID NO: 195 - protein, 7 aa, Artificial sequence
  Synthesized
QDSNLAS

SEQ ID NO: 196 - protein, 7 aa, Artificial sequence
  Synthesized
QGSNLAS

SEQ ID NO: 197 - protein, 7 aa, Artificial sequence
  Synthesized
QMSNRAS

SEQ ID NO: 198 - protein, 7 aa, Artificial sequence
  Synthesized
QGSNGAS

SEQ ID NO: 199 - protein, 7 aa, Artificial sequence
  Synthesized
QGSNTAS

SEQ ID NO: 200 - protein, 7 aa, Artificial sequence
  Synthesized
QKSNRAS

SEQ ID NO: 201 - protein, 7 aa, Artificial sequence
  Synthesized
QKSNGAS

SEQ ID NO: 202 - protein, 7 aa, Artificial sequence
  Synthesized
QISNRAS

SEQ ID NO: 203 - protein, 7 aa, Artificial sequence
  Synthesized
QISNGAS

SEQ ID NO: 204 - protein, 7 aa, Artificial sequence
  Synthesized
QESNRAS

SEQ ID NO: 205 - protein, 7 aa, Artificial sequence
  Synthesized
QESNGAS

SEQ ID NO: 206 - protein, 9 aa, Artificial sequence
  Synthesized
GQNLELPLT

SEQ ID NO: 207 - protein, 9 aa, Artificial sequence
  Synthesized
AQNDELPLT

SEQ ID NO: 208 - protein, 9 aa, Artificial sequence
  Synthesized
AQNEELPLT

SEQ ID NO: 209 - protein, 9 aa, Artificial sequence
  Synthesized
AQNGELPLT

SEQ ID NO: 210 - protein, 9 aa, Artificial sequence
  Synthesized
AQNQELPLT

SEQ ID NO: 211 - protein, 9 aa, Artificial sequence
  Synthesized
AQNTELPLT

SEQ ID NO: 212 - protein, 9 aa, Artificial sequence
  Synthesized
AQNIELPLT

SEQ ID NO: 213 - protein, 9 aa, Artificial sequence
  Synthesized
AQNLGLPLT

SEQ ID NO: 214 - protein, 112 aa, Artificial sequence
  Synthesized
QVQLVQSGAEVKKPGATVKISCKASGFNIKDDYMNWVQQAPGKGLEWIGWIDPENGDTEYASKFQGRATMTADTSTDTAYMELSSLRSEDTAVYYCTTSNGWGQGTLVTVSS

SEQ ID NO: 215 - protein, 112 aa, Artificial sequence
  Synthesized
EVQLVQSGAEVKKPGATVKISCKASGFNIKDDYMNWVQQAPGKGLEWIGWIDPENGDTEYASKFQGRATMTADTSTDTAYMELSSLRSEDTAVYYCTTSNGWGQGTLVTVSS

SEQ ID NO: 216 - protein, 112 aa, Artificial sequence
  Synthesized
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPLTFGGGTKVEIK

SEQ ID NO: 217 - protein, 112 aa, Artificial sequence
  Synthesized
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPLTFGGGTKLEIK

SEQ ID NO: 218 - protein, 117 aa, Homo sapiens
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCARIPLFGRDHWGQGTLVTVSS

SEQ ID NO: 219 - protein, 112 aa, Artificial sequence
  Synthesized
EVQLQQSGAELVRPGASVKLSCTASGFNIKDDYMNWVRQRPERGLEWIGWIDPENGDTAYASKFQGKATMTADTSSNTAYLQFSSLTSEDSAVYYCTTSNGWGQGTLVTVSA

SEQ ID NO: 220 - protein, 17 aa, Artificial sequence
  Synthesized
WIDPENGDTAYASKFQG

SEQ ID NO: 221 - protein, 112 aa, Artificial sequence
  Synthesized
QVQLVQSGAEVKKPGATVKISCKVSGFNIKDDYMNWVQQAPGKGLEWMGWIDPENGDTAYASKFQGRVTITADTSTDTAYMELSSLRSEDTAVYYCARSNGWGQGTLVTVSS

SEQ ID NO: 222 - protein, 112 aa, Artificial sequence
  Synthesized
EVQLVQSGAEVKKPGATVKISCKVSGFNIKDDYMNWVQQAPGKGLEWIGWIDPENGDTAYASKFQGRVTITADTSTDTAYMELSSLRSEDTAVYYCTTSNGWGQGTLVTVSS

SEQ ID NO: 223 - protein, 112 aa, Artificial sequence
  Synthesized
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPLTFGGGTKVEIK

SEQ ID NO: 224 - protein, 112 aa, Artificial sequence; identical to SEQ ID NO: 217
  Synthesized

SEQ ID NO: 225 - protein, 112 aa, Artificial sequence
  Synthesized
KIQLQQSGAELVRPGASVKLSCTASAFNIKDDYMNWVKQRPERGLEWIGWIDPENGDTKYASKFQGKATMTADTSSNTAYLQLSSLTSEDTAVYYCTTSNGWGQGTLVTVSA

SEQ ID NO: 226 - protein, 10 aa, Artificial sequence
  Synthesized
AFNIKDDYMN

SEQ ID NO: 227 - protein, 17 aa, Artificial sequence
  Synthesized
WIDPENGDTKYASKFQG

SEQ ID NO: 228 - protein, 112 aa, Artificial sequence
  Synthesized
DVVMTQTPLTLSVTIGQPASISCTSSQSLLHSNRKTYLHWLLQRPGQSPKLLIYLVSKLESGVPDRFSGSGSGTDFTLKISRVEAEDLGVYYCLQTTHFPRTFGGGTKLEIK

SEQ ID NO: 229 - protein, 16 aa, Artificial sequence
  Synthesized
TSSQSLLHSNRKTYLH

SEQ ID NO: 230 - protein, 7 aa, Artificial sequence
  Synthesized
LVSKLES

SEQ ID NO: 231 - protein, 9 aa, Artificial sequence
  Synthesized
LQTTHFPRT

SEQ ID NO: 232 - protein, 112 aa, Artificial sequence
  Synthesized
QIQLVQSGAEVKKPGATVKISCKASAFNIKDDYMNWVQQAPGKGLEWIGWIDPENGDTKYASKFQGRATMTADTSTDTAYMELSSLRSEDTAVYYCTTSNGWGQGTTVTVSR

SEQ ID NO: 233 - protein, 112 aa, Artificial sequence; identical to SEQ ID NO: 16
  Synthesized

SEQ ID NO: 234 - protein, 112 aa, Artificial sequence
  Synthesized
DVVMTQTPLSLSVTPGQPASISCTSSQSLLHSNRKTYLHWLLQKPGQPPQLLIYLVSKLESGVPDRFSGSGSGTDFTLKISRVEAEDVGVYYCLQTTHFPRTFGGGTKVEIK

SEQ ID NO: 235 - protein, 112 aa, Artificial sequence
  Synthesized
DVVMTQTPLSLSVTPGQPASISCTSSQSLLHSNRKTYLHWLLQKPGQSPQLLIYLVSKLESGVPDRFSGSGSGTDFTLKISRVEAEDVGVYYCLQTTHFPRTFGGGTKVEIK

SEQ ID NO: 236 - protein, 119 aa, Artificial sequence
  Synthesized
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 237 - protein, 112 aa, Artificial sequence
  Synthesized
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 238 - protein, 112 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLPPTFGGGTKVEIK

SEQ ID NO: 239 - protein, 112 aa, Homo sapiens
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLPLTFGGGTKVEIK

SEQ ID NO: 240 - protein, 114 aa, Artificial sequence
  Synthesized
QVTLKESGPGILQPSQTLSLTCSFSGFSLSTYGMGVGWIRQPSGKGLEWLANIWWDDIKYYNAALKSRLTISKDTSKNQVFLKIASVDTADTATYYCARNVDYWGQGTTLTVSS

SEQ ID NO: 241 - protein, 12 aa, Artificial sequence
  Synthesized
GFSLSTYGMGVG

SEQ ID NO: 242 - protein, 16 aa, Artificial sequence
  Synthesized
NIWWDDIKYYNAALKS

SEQ ID NO: 243 - protein, 4 aa, Artificial sequence
  Synthesized
NVDY

SEQ ID NO: 244 - protein, 112 aa, Artificial sequence
  Synthesized
DVVMTQTPLSLPVSLGDQASISCRSSQSLVHSNGNTFLHWYLQKPGQSPKLLIYKVSNRFSGVPDRFSGSGSGTDFTLKISRVEAEDLGVYFCSQSTLVPWTFGGGTKLEIK

SEQ ID NO: 245 - protein, 16 aa, Artificial sequence
  Synthesized
RSSQSLVHSNGNTFLH

SEQ ID NO: 246 - protein, 7 aa, Artificial sequence
  Synthesized
KVSNRFS

SEQ ID NO: 247 - protein, 9 aa, Artificial sequence
  Synthesized
SQSTLVPWT

SEQ ID NO: 248 - protein, 114 aa, Artificial sequence
  Synthesized
QVTLKESGPALVKPTQTLTLTCTFSGFSLSTYGMGVSWIRQPPGKALEWLANIWWDDIKYYNAALKSRLTISKDTSKNQVVLTMTNMDPVDTATYYCARNVDYWGQGTMVTVSL

SEQ ID NO: 249 - protein, 114 aa, Artificial sequence
  Synthesized
QVTLKESGPALVKPTQTLTLTCTFSGFSLSTYGMGVSWIRQPPGKALEWLANIWWDDIKYYNAALKSRLTISKDTSKNQVVLTMTNMDPVDTATYYCARNVDYWGQGTLVTVSS

SEQ ID NO: 250 - protein, 112 aa, Artificial sequence
  Synthesized
DVVMTQTPLSLPVTPGEPASISCRSSQSLVHSNGNTFLHWYLQKPGQSPQLLIYKVSNRFSGVPDRFSGSGSGTDFTLKISRVEAEDVGVYFCSQSTLVPWTFGGGTKVEIK

SEQ ID NO: 251 - protein, 112 aa, Artificial sequence
  Synthesized
DVVMTQSPLSLPVTPGEPASISCRSSQSLVHSNGNTFLHWYQQKPGQSPQLLIYKVSNRFSGVPDRFSGSGSGTDFTLKISRVEAEDVGVYFCSQSTLVPWTFGQGTKLEIK

SEQ ID NO: 252 - protein, 118 aa, Artificial sequence
  Synthesized
QITLEESGPGILQPSQTLSLTCSFSGFSLSSSAMSVGWIRQPSGKGLEWLAHIWWNDDKYYNPALKSRLTVSKDSSDNQVFLKIASVVTADTATYYCARIPGFGFDYWGQGTTLTVSS

SEQ ID NO: 253 - protein, 119 aa, Homo sapiens
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCARLAVADAFDIWGQGTMVTVSL

SEQ ID NO: 254 - protein, 113 aa, Homo sapiens
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCARNDYWGQGTLVTVSS

SEQ ID NO: 255 - protein, 112 aa, Artificial sequence
  Synthesized
DVVMTQTPLSLSVSLGDQASISCRSSQSLVHSNGNTFLQWYLQKPGQSPKLLIYKVSNRFSGVPDRFSGSGSGTDFTLKISRMEAEDLGIYFCSQTTHVPWTFGGGTKLEIK

SEQ ID NO: 256 - protein, 112 aa, Homo sapiens
DIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 257 - protein, 10 aa, Artificial sequence
  Synthesized
WIDPENGDTA

SEQ ID NO: 258 - protein, 13 aa, Artificial sequence
  Synthesized
WIGWIDPENGDTA

SEQ ID NO: 259 - protein, 7 aa, Artificial sequence
  Synthesized
AFNIKDD

SEQ ID NO: 260 - protein, 10 aa, Artificial sequence
  Synthesized
WIDPENGDTK

SEQ ID NO: 261 - protein, 13 aa, Artificial sequence
  Synthesized
WIGWIDPENGDTK

SEQ ID NO: 262 - protein, 7 aa, Artificial sequence
  Synthesized
KTYLHWL

SEQ ID NO: 263 - protein, 10 aa, Artificial sequence
  Synthesized
LLIYLVSKLE

SEQ ID NO: 264 - protein, 8 aa, Artificial sequence
  Synthesized
LQTTHFPR

SEQ ID NO: 265 - protein, 7 aa, Artificial sequence
  Synthesized
TYGMGVG

SEQ ID NO: 266 - protein, 9 aa, Artificial sequence
  Synthesized
GFSLSTYGM

SEQ ID NO: 267 - protein, 5 aa, Artificial sequence
  Synthesized
WWDDI

SEQ ID NO: 268 - protein, 9 aa, Artificial sequence
  Synthesized
NIWWDDIKY

SEQ ID NO: 269 - protein, 8 aa, Artificial sequence
  Synthesized
STYGMGVG

SEQ ID NO: 270 - protein, 12 aa, Artificial sequence
  Synthesized
WLANIWWDDIKY

SEQ ID NO: 271 - protein, 5 aa, Artificial sequence
  Synthesized
ARNVD

SEQ ID NO: 272 - protein, 7 aa, Artificial sequence
  Synthesized
NTFLHWY

SEQ ID NO: 273 - protein, 10 aa, Artificial sequence
  Synthesized
LLIYKVSNRF

SEQ ID NO: 274 - protein, 8 aa, Artificial sequence
  Synthesized
SQSTLVPW

SEQ ID NO: 275 - protein, 12 aa, Artificial sequence
  Synthesized
GFSLSTYGMGVS

SEQ ID NO: 276 - protein, 4 aa, Artificial sequence
  Synthesized
IVYK

SEQ ID NO: 277 - protein, 6 aa, Artificial sequence
  Synthesized
EIVYKS

(19) United States
(12) Patent Application Publication
(10) Pub. No.: US 2022/0275067 A1
(43) Pub. Date: Sep. 1, 2022

(54) ANTIBODIES RECOGNIZING TAU

(71) Applicant: PROTHENA BIOSCIENCES LIMITED, Dublin 2 (IE)

(72) Inventors: Philip James DOLAN, III, Foster City, CA (US); Tarlochan S. NIJJAR, Orinda, CA (US); Svetlana ALEXANDER, Sunnyvale, CA (US); Robin BARBOUR, Walnut Creek, CA (US); Stephen Jed TAM, Belmont, CA (US)

(73) Assignee: PROTHENA BIOSCIENCES LIMITED, Dublin 2 (IE)

(21) Appl. No.: 17/429,288

(22) PCT Filed: Feb. 7, 2020
(86) PCT No.: PCT/US2020/017357

§ 371 Date: Aug. 6, 2021

Related U.S. Application Data
(60) Provisional application No. 62/803,334, filed on Feb. 8, 2019.
(60) Provisional application No. 62/813,124, filed on Mar. 3, 2019.
(60) Provisional application No. 62/855,434, filed on May 31, 2019.

(51) Int. Cl. C07K 16/18

(52) U.S. Cl. CPC C07K 16/18; C07K 2317/565; C07K 2317/24; C07K 2317/77; C07K 2317/92; C07K 2317/76; C07K 2317/567

(57) ABSTRACT

The invention provides antibodies that specifically bind tau. The antibodies inhibit or delay tau-associated pathologies and associated symptomatic deterioration.

### CROSS REFERENCE TO RELATED APPLICATIONS

This application claims the benefit under 35 USC 119(e) of U.S. Provisional Application No. 62/803,334, filed Feb. 8, 2019, U.S. Provisional Application No. 62/813,124, filed Mar. 3, 2019, and U.S. Provisional Application No. 62/855,434, filed May 31, 2019, each of which is incorporated by reference in its entirety for all purposes.

### REFERENCE TO A SEQUENCE LISTING

The Sequence Listing written in file 2020_02_07_542496WO_SEQLST.txt is 3560 kilobytes, was created on Feb. 7, 2020, and is hereby incorporated by reference.

### BACKGROUND OF THE INVENTION

Tau is a well-known human protein that can exist in phosphorylated forms (see, e.g., Goedert, Proc. Natl. Acad. Sci. U.S.A. 85:4051-4055(1988); Goedert, EMBO J. 8:393-399(1989); Lee, Neuron 2:1615-1624(1989); Goedert, Neuron 3:519-526(1989); Andreadis, Biochemistry 31:10626-10633(1992). Tau has been reported to have a role in stabilizing microtubules, particularly in the central nervous system. Total tau (t-tau, i.e., phosphorylated and unphosphorylated forms) and phospho-tau (p-tau, i.e., phosphorylated tau) are released by the brain in response to neuronal injury and neurodegeneration and have been reported to occur at increased levels in the CSF of Alzheimer's patients relative to the general population (Jack et al., Lancet Neurol 9: 119-28 (2010)).

Tau is the principal constituent of neurofibrillary tangles, which together with plaques are a hallmark characteristic of Alzheimer's disease. The tangles constitute abnormal fibrils measuring 10 nm in diameter occurring in pairs wound in a helical fashion with a regular periodicity of 80 nm. The tau within neurofibrillary tangles is abnormally phosphorylated (hyperphosphorylated) with phosphate groups attached to specific sites on the molecule. Severe involvement of neurofibrillary tangles is seen in the layer II neurons of the entorhinal cortex, the CA1 and subicular regions of the hippocampus, the amygdala, and the deeper layers (layers III, V, and superficial VI) of the neocortex in Alzheimer's disease. Hyperphosphorylated tau has also been reported to interfere with microtubule assembly, which may promote neuronal network breakdown.

Tau inclusions are part of the defining neurophathology of several neurodegenerative diseases including Alzheimer's disease, frontotemporal lobar degeneration, progressive supranuclear palsy and Pick's disease.

### BRIEF SUMMARY OF THE CLAIMED INVENTION

In one aspect, the invention provide an isolated monoclonal antibody that competes for binding to human tau with antibody 9F5. In some such antibodies, the heavy chain CDR-H3 has an amino acid sequence comprising SEQ ID NO:10. In some such antibodies, the heavy chain CDR-H1 has an amino acid sequence comprising SEQ ID NO:8. In some such antibodies, the light chain CDRs CDR-L1, CDR-L2 and CDR-L3 have amino acid sequences comprising SEQ ID NO:12, 13 and 14, respectively). in some such antibodies, the heavy chain CDR-H1 has an amino acid sequence comprising SEQ ID NO: 8. Some such antibodies bind to the same epitope on human tau as 9F5.

Some such antibodies comprise three light chain CDRs and three heavy chain CDRs of monoclonal antibody 9F5, wherein 9F5 is a mouse antibody characterized by a heavy chain variable region having an amino acid sequence comprising SEQ ID NO:7 and a light chain variable region having an amino acid sequence comprising SEQ ID NO:11.

In some such antibodies, the three heavy chain CDRs CDR-H1, CDR-H2, and CDR-H3are as defined by Kabat/Chothia Composite (SEQ ID NOs:8, 9, and 10, respectively), except that position H28 can be occupied by N or T, position H51 can be occupied by I or V, position H54 can be occupied by N or D, and position H56 can be occupied by D or E, and the three light chain CDRs CDR-L1, CDR-L2, and CDR-L3 are as defined by Kabat/Chothia Composite (SEQ ID NOs:12, 13, and 14, respectively), except that position L27b is occupied by L, D, T, or Q, position L27c is occupied by L, D, G, S, E, T, N, A, P, or I, position L30 can be occupied by I, Y, E, K, G, or Q, position L31 can be occupied by T, N, or G, position L33 is occupied by L N, T, S, R, or G, position L51 can be occupied by M, G, E, D, K, or I, position L54 can be occupied by L, R, G, or T, position L89 is occupied by A or G, position L92 is occupied by L, D, E, G, Q, T, or I, and position L93 is occupied by E or G.

In some antibodies, CDR-H1 has an amino acid sequence comprising SEQ ID NO:50. In some antibodies, CDR-H2 has an amino acid sequence comprising SEQ ID NO:51. In some antibodies, CDR-H2 has an amino acid sequence comprising SEQ ID NO:52. In some antibodies, CDR-L1 has an amino acid sequence comprising any of SEQ ID NO:53, SEQ ID NO:54, and SEQ ID NOs:172-193. In some antibodies, CDR-L2 has an amino acid sequence comprising any of SEQ ID NO:55 and SEQ ID NOs:194-205. In some antibodies, CDR-L3 has an amino acid sequence comprising any of SEQ ID NOs:206-213. In some antibodies, CDR-H1 has an amino acid sequence comprising SEQ ID NO:50 and CDR-H2 has an amino acid sequence comprising SEQ ID NO:51. In some antibodies, CDR-L1 has an amino acid sequence comprising SEQ ID NO:53 and CDR-L2 has an amino acid sequence comprising SEQ ID NO:55. In some antibodies, CDR-L1 has an amino acid sequence comprising SEQ ID NO:54 and CDR-L2 has an amino acid sequence comprising SEQ ID NO:55.

Some antibodies are 9F5 or a chimeric, veneered, or humanized form thereof. In some antibodies, the variable heavy chain has ≥85% identity to human sequence. In some antibodies, the variable light chain has ≥85% identity to human sequence. In some antibodies, each of the variable heavy chain and variable light chain has ≥85% identity to human germline sequence. Some antibodies are humanized antibodies.

Some antibodies are a humanized or chimeric 9F5 antibody that specifically binds to human tau, wherein 9F5 is a mouse antibody characterized by a mature heavy chain variable region of SEQ ID NO:7 and a mature light chain variable region of SEQ ID NO:11. Some antibodies comprise a humanized mature heavy chain variable region comprising the three heavy chain CDRs of 9F5 and a humanized mature light chain variable region comprising the three light chain CDRs of 9F5. In some antibodies, the CDRs are of a definition selected from the group of Kabat, Chothia, Kabat/Chothia Composite, AbM and Contact.

In some antibodies, the humanized mature heavy chain variable region comprises the three Kabat/Chothia Composite heavy chain CDRs of 9F5 (SEQ ID NOs:8-10) and the humanized mature light chain variable region comprises the three Kabat/Chothia Composite light chain CDRs of 9F5 (SEQ ID NOs:12-14). In some antibodies, the humanized mature heavy chain variable region comprises the three Kabat heavy chain CDRs of 9F5 (SEQ ID NO:40, SEQ ID NO:9, and SEQ ID NO:10) and the humanized mature light chain variable region comprises the three Kabat light chain CDRs of 9F5 (SEQ ID NOs:12-14). In some antibodies, the humanized mature heavy chain variable region comprises the three Chothia heavy chain CDRs of 9F5 (SEQ ID NO:41, SEQ ID NO:42, and SEQ ID NO:10) and the humanized mature light chain variable region comprises the three Chothia light chain CDRs of 9F5 (SEQ ID NOs:12-14). In some antibodies, the humanized mature heavy chain variable region comprises the three AbM heavy chain CDRs of 9F5 (SEQ ID NO:8, SEQ ID NO:43, and SEQ ID NO:10) and the humanized mature light chain variable region comprises the three AbM light chain CDRs of 9F5 (SEQ ID NOs:12-14). In some antibodies, the humanized mature heavy chain variable region comprises the three Contact heavy chain CDRs of 9F5 (SEQ ID NO:44-46) and the humanized mature light chain variable region comprises the three Contact light chain CDRs of 9F5 (SEQ ID NO:47-49).

For example, the antibody can be a humanized antibody, veneered antibody, or chimeric antibody.

Some such antibodies comprise a humanized mature heavy chain variable region having an amino acid sequence at least 90% identical to any one of SEQ ID NOs:15-22 and SEQ ID NOs:109-129, and a humanized mature light chain variable region having an amino acid sequence at least 90% identical to any one of SEQ ID NOs:23-29, SEQ ID NOs:61-108, and SEQ ID NOs:130-171.

In some antibodies, at least one of the following positions in the VH region is occupied by the amino acid as specified: H1 is occupied by E, H17 is occupied by T, H20 is occupied by I, H69 is occupied by M, H75 is occupied by T, H93 is occupied by T, H94 is occupied by T, and H109 is occupied by V. In some antibodies, positions H1, H17, H20, H69, H75, H94, and H109 are occupied by E, T, I, M, T, T, T, and V, respectively.

In some antibodies, at least one of the following positions in the VH region is occupied by the amino acid as specified: H66 is occupied by R and H81 is occupied by E. In some antibodies, positions H66 and H81 are occupied by R and E, respectively.

In some antibodies, at least one of the following positions in the VH region is occupied by the amino acid as specified: H23 is occupied by I and H83 is occupied by R. In some antibodies, positions H23 and H83 are occupied by K and R, respectively.

In some antibodies, at least one of the following positions in the VH region is occupied by the amino acid as specified: H43 is occupied by K, H51 is occupied by V, H76 is occupied by D, M80 is occupied by M, and H108 is occupied by L. In some antibodies, positions H43, H51, H76, H80, and H108 are occupied by K, V, D, M, and L, respectively.

In some antibodies, position H28 in the VH region is occupied by T.

In some antibodies, at least one of the following positions in the VH region is occupied by the amino acid as specified: H54 is occupied by D and H56 is occupied by E. In some antibodies, positions H54 and H56 are occupied by D and E, respectively.

In some antibodies, position H40 in the VH region is occupied by A.

In some antibodies, at least one of the following positions in the VH region is occupied by the amino acid as specified: H5 is occupied by V, H11 is occupied by V, H12 is occupied by K, H38 is occupied by R, and H42 is occupied by G. In some antibodies, positions H5, H11, H12, H38, and H42 are occupied by V, V, K, R, and G, respectively.

In some antibodies, at least one of the following positions in the VH region is occupied by the amino acid as specified: H1 is occupied by Q or E, H5 is occupied by Q or V, H11 is occupied by L or V, H12 is occupied by V or K, H17 is occupied by S or T, H20 is occupied by L or I, H23 is occupied by T or K, H28 is occupied by N or T, H38 is occupied by K, R, or Q, H40 is occupied by R or A, H42 is occupied by E or G, H43 is occupied by Q or K, H48 is occupied by I or M, H51 is occupied by I or V, H54 is occupied by N or D, H56 is occupied by D or E, H66 is occupied by K or R, H69 is occupied by I or M, H75 is occupied by S or T, H76 is occupied by N or D, H79 is occupied by Y, Q, D, N, or G, H80 is occupied by L, M, P, D, G, or E, H81 is occupied by Q or E, H82 is occupied by L, P, K, R, E, or N, H82a is occupied by S or G, H82c is occupied by L, G, D, or S, H83 is occupied by T or R, H93 is occupied by A or T, H94 is occupied by S or T, H108 is occupied by T or L, H109 is occupied by L or V.

In some antibodies, positions H1, H17, H20, H69, H75, H93, H94, and H109 in the VH region are occupied by E, T, I, M, T, T, T, and V, respectively. In some antibodies, positions H1, H17, H20, H66, H69, H75, H81, H93, H94, and H109 in the VH region are occupied by E, T, I, R, M, T, E, T, T, and V, respectively. In some antibodies, positions H1, H17, H20, H23, H28, H66, H69, H75, H81, H83, H93, H94, and H109 in the VH region are occupied by E, T, I, K, T, R, M, T, E, R, T, T, and V, respectively. In some antibodies, positions H1, H17, H20, H23, H28, H43, H51, H54, H56, H66, H69, H75, H76, H80, H81, H83, H93, H94, H108, and H109 in the VH region are occupied by E, T, I, K, T, K, V, D, E, R, M, T, D, M, E, R, T, T, L, and V, respectively. In some antibodies, positions H1, H17, H20, H23, H28, H40, H43, H48, H51, H54, H56, H66, H69, H75, H76, H80, H81, H83, H93, H94, H108, and H109 in the VH region are occupied by E, T, I, K, T, A, K, M, V, D, E, R, M, T, D, M, E, R, T, T, L, and V, respectively. In some antibodies, positions H1, H5, H11, H12, H17, H20, H23, H38, H40, H42, H43, H51, H54, H56, H66, H69, H75, H76, H80, H81, H83, H93, H94, H108, and H109 in the VH region are occupied by E, V, V, K, T, I, K, R, A, G, K, V, D, E, R, M, T, D, M, E, R, T, T, L, and V, respectively. In some antibodies, H1, H5, H11, H12, H17, H20, H23, H38, H40, H42, H43, H51, H66, H69, H75, H76, H80, H81, H83, H93, H94, H108, and H109 in the VH region are occupied by E, V, V, K, T, I, K, R, A, G, K, V, R, M, T, D, M, E, R, T, T, L, and V, respectively.

In some antibodies, positions H1, H5, H11, H12, H17, H20, H23, H38, H42, H43, H66, H69, H75, H80, H81, H83, H93, H94, H108, and H109 in the VH region are occupied by E, V, V, K, T, I, K, Q, G, K, R, M, T, M, E, R, T, T, L, and V, respectively. In some antibodies, the heavy chain variable region comprises the amino acid sequence of SEQ ID NO:127.

In some antibodies, positions H1, H5, H11, H12, H17, H20, H23, H38, H42, H43, H66, H69, H75, H80, H81, H83, H93, H94, H108, and H109 in the VH region are occupied by E, V, V, K, T, I, K, K, E, K, R, M, T, M, E, R, T, T, L, and V, respectively. In some antibodies, the heavy chain variable region comprises the amino acid sequence of SEQ ID NO:128.

In some antibodies, positions H1, H5, H11, H12, H17, H20, H23, H38, H42, H43, H66, H69, H75, H80, H81, H82c, H83, H93, H94, H108, and H109 in the VH region are occupied by E, V, V, K, T, I, K, K, E, K, R, M, T, M, E, G, R, T, T, L, and V, respectively.

In some antibodies, position H80 in the VH region is occupied by P. In some antibodies, position H80 in the VH region is occupied by D. In some antibodies, position H82c in the VH region is occupied by G. In some antibodies, position H82c in the VH region is occupied by D. In some antibodies, position H82 in the VH region is occupied by P. In some antibodies, position H80 in the VH region is occupied by G. In some antibodies, position H82 in the VH region is occupied by K. In some antibodies, position H82 in the VH region is occupied by R. In some antibodies, position H82 in the VH region is occupied by E. In some antibodies, position H82 in the VH region is occupied by N.

In some antibodies, position H79 in the VH region is occupied by D. In some antibodies, position H79 in the VH region is occupied by N. In some antibodies, position H79 in the VH region is occupied by G. In some antibodies, position H80 in the VH region is occupied by E. In some antibodies, position H80 in the VH region is occupied by G. In some antibodies, position H82c in the VH region is occupied by S. In some antibodies, position H79 in the VH region is occupied by Q. In some antibodies, position H82a in the VH region is occupied by G.

In some antibodies, at least one of the following positions in the VL region is occupied by the amino acid as specified: L7 is occupied by S, L8 is occupied by P, L15 is occupied by P, and L100 is occupied by Q. In some antibodies, positions L7, L8, L15, and L100 are occupied by S, P, P, and Q, respectively.

In some antibodies, position L66 in the VL region is occupied by G. In some antibodies, position L64 in the VL region is occupied by S.

In some antibodies, position L17 in VL region is occupied by E. In some antibodies, at least one of the following positions in the VL region is occupied by the amino acid as specified: L11 is occupied by L, L51 is occupied by G, and L54 is occupied by R.

In some antibodies, positions L11, L51, and L54 are occupied by L, G, and R, respectively. In some antibodies, position L30 in the VL region is occupied by Y.

In some antibodies, at least one of the following positions in the VL region is occupied by the amino acid as specified: L3 is V or Q, L7 is A or S, L8 is A or P, L9 is F or L, L11 is N or L, L15 is L or P, L17 is T or E, L18 is S or P, L27b is L, D. T, or Q, L27c is L, D, G, S, E, T, N, A, P, or I, L30 is I,Y, E, K, G, L31 is T, N, or G, L33 is L, N, T, S, R, or G, L37 is L, Q, G, or I, L39 is R or K, L51 is M, G, E, D, K, or I, L54 is R, G, or T, L60 is N or D, L64 is G or S, L66 is E or G, L73 is L, P, or G, L74 is R or K, L75 is I, D, P, Q, or G, L76 is S, P, or G, L77 is R or D, L78 is V, R, D, E, P, K, G, or Q, L85 is V or G, L86 is Y or T, L89 is A or G, L92 is L, D, E, G, Q, T, or I, L93 is E or G , L100 is G or Q.

In some antibodies, positions L64 and L66 in the VL region are occupied by S and G, respectively. In some antibodies, positions L7, L8, L15, L64, L66, and L100 in the VL region are occupied by S, P, P, S, G, and Q, respectively. In some antibodies, positions L7, L8, L15, L17, L66, and L100 in the VL region are occupied by S, P, P, E, G, and Q, respectively. In some antibodies, positions L7, L8, L11, L15, L17, L51, L54, L66, and L100 in the VL region are occupied by S, P, L, P, E, G, R, G, and Q, respectively.

In some antibodies, the light chain variable region comprises the amino acid sequence of any of SEQ ID NOs:133, 135-137, 142-144, 149, 158, 159 and 168. In some antibodies, the light chain variable region comprises the amino acid sequence of SEQ ID NO:133. In some antibodies, the light chain variable region comprises the amino acid sequence of SEQ ID NO:137. In some antibodies, the light chain variable region comprises the amino acid sequence of SEQ ID NO:149. In some antibodies, the light chain variable region comprises the amino acid sequence of SEQ ID NO:159.

In some antibodies, positions L7, L8, L11, L15, L17, L30, L51, L54, L66, and L100 in the VL region are occupied by S, P, L, P, E, Y, G, R, G, and Q, respectively. In some antibodies, positions L7, L8, L11, L15, L17, L30, L51, L54, and L100 in the VL region are occupied by S, P, L, P, E, Y, G, R, and Q, respectively. In some antibodies, positions L7, L8, L9, L11, L15, L17, L18, L31, L39, L51, L54, L60, L66, L74, and L100 in the VL region are occupied by S, P, L, L, P, E, P, N, K, G, R, D, G, K, and Q, respectively.

In some antibodies, positions L7, L8, L11, L15, L17, L39, L60, L64, L66, L74, and L100 in the VL region are occupied by S, P, L, P, E, K, N, S, G, K and Q, respectively. In some antibodies, position L3 in the VL region is occupied by Q. In some antibodies, position L27c in the VL region is occupied by D, G, I, L or S, position L37 in the VL region is occupied by G, I, L, or Q, position L51 in the VL region is occupied by E, G, I, K or M, position L54 in the VL region is occupied by G, L, R or T, and position L92 in the VL region is occupied by G, I or L. In some antibodies, position L27c in the VL region is occupied by D or S, position L37 in the VL region is occupied by G, L or Q, position L51 in the VL region is occupied by G or K, position L54 in the VL region is occupied by R, and position L92 in the VL region is occupied by I.

In some antibodies, position L27c in the VL region is occupied by D, position L37 in the VL region is occupied by G, and position L51 in the VL region is occupied by G. In some antibodies, the heavy chain variable region has an amino acid sequence comprising SEQ ID NO:127 and the light chain variable region has an amino acid sequence comprising SEQ ID NO:149.

In some antibodies, position L27c in the VL region is occupied by D, position L37 in the VL region is occupied by Q, and position L51 in the VL region is occupied by G. In some antibodies, the heavy chain variable region has an amino acid sequence comprising SEQ ID NO:127 and the light chain variable region has an amino acid sequence comprising SEQ ID NO:137.

In some antibodies, position L27c in the VL region is occupied by S, position L37 in the VL region is occupied by L and position L51 in the VL region is occupied by G. In some antibodies, the heavy chain variable region has an amino acid sequence comprising SEQ ID NO:127 and the light chain variable region has an amino acid sequence comprising SEQ ID NO:159.

In some antibodies, position L27c in the VL region is occupied by D, position L37 in the VL region is occupied by Q, and position L51 in the VL region is occupied by K. In some antibodies, the heavy chain variable region has an amino acid sequence comprising SEQ ID NO:127 and the light chain variable region has an amino acid sequence comprising SEQ ID NO:138.

In some antibodies, position L27c in the VL region is occupied by S, position L37 in the VL region is occupied by Q, and position L51 in the VL region is occupied by G. In some antibodies, the heavy chain variable region has an amino acid sequence comprising SEQ ID NO:127 and the light chain variable region has an amino acid sequence comprising SEQ ID NO:133.

In some antibodies, positions L7, L8, L11, L15, L17, L39, L60, L64, L66, L74, and L100 in the VL region are occupied by, respectively S, P, L, P, E, K, D, S, G, K, and Q. In some antibodies, position L3 in the VL region is occupied by Q. In some antibodies, position L27c in the VL region is occupied by G or S, position L37 in the VL region is occupied by G, I or Q, position L51 in the VL region is occupied by G, I or K, position L54 in the VL region is occupied by G or R, and position L92 in the VL region is occupied by G, I, or L.

In some antibodies, position L27c in the VL region is occupied by G, position L37 in the VL region is occupied by G, position L51 in the VL region is occupied by G, and position L54 in the VL region is occupied by R. In some antibodies, position L92 in the VL region is occupied by I. In some antibodies, the heavy chain variable region has an amino acid sequence comprising SEQ ID NO:129 and the light chain variable region has an amino acid sequence comprising SEQ ID NO:168.

In some antibodies, position L51 in the VL region is occupied by E. In some antibodies, position L51 in the VL region is occupied by D. In some antibodies, position L27c in the VL region is occupied by D. In some antibodies, position L27c in the VL region is occupied by G. In some antibodies, position L27c in the VL region is occupied by S. In some antibodies, position L27c in the VL region is occupied by E. In some antibodies, position L30 in the VL region is occupied by E. In some antibodies, position L30 in the VL region is occupied by K. In some antibodies, position L27c in the VL region is occupied by T.

In some antibodies, position L27c in the VL region is occupied by N. In some antibodies, position L27b in the VL region is occupied by D. In some antibodies, position L30 in the VL region is occupied by G. In some antibodies, provided position L33 in the VL region is occupied by N. In some antibodies, position L27c in the VL region is occupied by A. In some antibodies, position L33 in the VL region is occupied by T. In some antibodies, position L33 in the VL region is occupied by S. In some antibodies, position L33 in the VL region is occupied by R. In some antibodies, position L30 in the VL region is occupied by Q. In some antibodies, position L27b in the VL region is occupied by T.

In some antibodies, position L31 in the VL region is occupied by G. In some antibodies, position L27b in the VL region is occupied by Q In some antibodies, position L33 in the VL region is occupied by G. In some antibodies, position L27c in the VL region is occupied by P. In some antibodies, position L78 in the VL region is occupied by R. In some antibodies, position L75 in the VL region is occupied by D. In some antibodies, position L78 in the VL region is occupied by D. In some antibodies, position L78 in the VL region is occupied by E. In some antibodies, position L78 in the VL region is occupied by P. In some antibodies, position L78 in the VL region is occupied by K.

In some antibodies, position L77 in the VL region is occupied by D. In some antibodies, position L78 in the VL region is occupied by G. In some antibodies, position L76 in the VL region is occupied by P. In some antibodies, position L75 in the VL region is occupied by P. In some antibodies, provided position L75 in the VL region is occupied by Q. In some antibodies, position L75 in the VL region is occupied by G. In some antibodies, position L73 in the VL region is occupied by P. In some antibodies, position L73 in the VL region is occupied by G. In some antibodies, position L78 in the VL region is occupied by Q. In some antibodies, position L76 in the VL region is occupied by G.

In some antibodies, position L92 in the VL region is occupied by D. In some antibodies, position L86 in the VL region is occupied by T. In some antibodies, position L92 in the VL region is occupied by E. In some antibodies, position L92 in the VL region is occupied by G. In some antibodies, position L92 in the VL region is occupied by Q. In some antibodies, position L93 in the VL region is occupied by G. In some antibodies, provided position L85 in the VL region is occupied by G. In some antibodies, position L92 in the VL region is occupied by T. In some antibodies, position L89 in the VL region is occupied by G.

In some antibodies, positions L3, L27c, L37, L51, L54, and L92 in the VL region are occupied by Q, S, Q, G, G, and I, respectively. In some antibodies, positions L3Q, L27c, L37, L51 L54, and L92 in the VL region are occupied by Q, S, Q, G, R, and I. In some antibodies, positions L3, L27c, L37, L51, L54, and L92 in the VL region are occupied by Q, S, Q, G, T, and I, respectively. In some antibodies, positions L3, L27c, L37, L51, L54, and L92 in the VL region are occupied by Q, S, Q, G, R, and G, respectively. In some antibodies, positions L3, L27c, L37, L51, L54, and L92 in the VL region are occupied by Q, G, Q, G, R, and I, respectively.

In some antibodies, positions L3, L27c, L37, L51, L54, and L92 in the VL region are occupied by Q, D, Q, G, R, and I, respectively. In some antibodies, positions L3, L27c, L37, L51, L54, and L92 in the VL region are occupied by Q, D, Q, K, R, and I, respectively. In some antibodies, positions L3, L27c, L37, L51, L54, and L92 in the VL region are occupied by Q, G, Q, K, R, and I, respectively. In some antibodies, positions L3, L27c, L37, L51, L54, and L92 in the VL region are occupied by Q. G, Q, K, G, and I, respectively. In some antibodies, positions L3, L27c, L37, L51, L54, and L92 in the VL region are occupied by Q, S, Q, K, G, and I, respectively.

In some antibodies, positions L3, L27c, L37, L51, L54, and L92 in the VL region are occupied by Q, G, G, G, R, and I, respectively. In some antibodies, positions L3, L27c, L37, L51, L54, and L92 in the VL region are occupied by Q, G, G, G, R, and G, respectively. In some antibodies, positions L3, L27c, L37, L51, and L54 in the VL region are occupied by Q, G, G, G, and R, respectively. In some antibodies, positions L3, L27c, L37, L51, L54, and L92 in the VL region are occupied by Q, G, G, G, T, and I. In some antibodies, positions L3, L27c, L37, L51, L54, and L92 in the VL region are occupied by Q, G, G, G, T, and G, respectively.

In some antibodies, positions L3, L27c, L37, L51, and L54 in the VL region are occupied by Q, G, G, G, and T, respectively. In some antibodies, positions L3, L27c, L37, L51, L54, and L92 in the VL region are occupied by Q, S, G, G, T, and I, respectively. In some antibodies, positions L3, L27c, L37, L51, L54, and L92 in the VL region are occupied by Q, D, G, G, R, and I, respectively. In some antibodies, positions L3, L27c, L37, L51, L54, and L92 in the VL region are occupied by Q, S, I, I, R, and I, respectively. In some antibodies, positions L3, L27c, L37, L51, L54, and L92 in the VL region are occupied by Q, S, Q, I, G, and I, respectively.

In some antibodies, positions L3, L27c, L37, L51, and L54 in the VL region are occupied by Q, S, Q, I, and G, respectively. In some antibodies, positions L3, L27c, L37, L51, L54, and L92 in the VL region are occupied by Q, S, Q, E, R, and I, respectively. In some antibodies, positions L3, L27c, L37, L51, L54, and L92 in the VL region are occupied by Q, G, Q, E, G, and I, respectively. In some antibodies, positions L3, L27c, L37, L51, L54, and L92L in the VL region are occupied by Q, G, I, E, R, and I, respectively. In some antibodies, positions L3, L27c, L37, L51, L54, and L92 in the VL region are occupied by Q, G, I, E, R. and G, respectively.

In some antibodies, positions L3, L27c, L37, L51, and L54 in the VL region are occupied by Q, I, I, E, and R, respectively. In some antibodies, positions L3, L37, L51, L54, and L92 in the VL region are occupied by Q, Q, G, R, and I, respectively. In some antibodies, positions L3, L27c, L51, L54, and L92 in the VL region are occupied by Q, S, G, R, and I, respectively. In some antibodies, positions L3, L27c, L37, L54, and L92 in the VL region are occupied by Q, S, Q, R, and I, respectively. In some antibodies, positions L3, L27c, L37, L51, and L92 in the VL region are occupied by Q, S, Q, G, and I, respectively.

In some antibodies, positions L3, L27c, L37, L51, L54, and L92 in the VL region are occupied by Q, S, Q, G, G, and I. In some antibodies, positions L3, L27c, L37, L51, L54, and L92 in the VL region are occupied by Q, S, Q, G, R, and I, respectively In some antibodies, positions L3, L27c, L37, L51, L54, and L92 in the VL region are occupied by Q, S, Q, G, R, and G, respectively. In some antibodies, positions L3, L27c, L37, L51, L54, and L92 in the VL region are occupied by Q, G, Q, G, R, and I, respectively. In some antibodies, positions L3, L27c, L37, L51, L54, and L92 in the VL region are occupied by Q, G, Q, K, R, and I , respectively.

In some antibodies, positions L3, L27c, L37, L51, L54, and L92 in the VL region are occupied by Q, S, Q, K, G, and I, respectively. In some antibodies, positions L3, L27c, L37, L51, L54, and L92 in the VL region are occupied by Q, G, G, G, R, and I, respectively. In some antibodies, positions L3, L27c, L37, L51, and L54 in the VL region are occupied by Q, G, G, G, and R, respectively. In some antibodies, positions L3, L27c, L37, L51, L54, and L92 in the VL region are occupied by Q, S, I, I, R, and I. In some antibodies, positions L3, L27c, L37, L51, L54, and L92 in the VL region are occupied by Q, S, Q, I, G, and I, respectively.

Some antibodies comprise a mature heavy chain variable region having an amino acid sequence at least 95% identical to any one of SEQ ID NOs:15-22 and SEQ ID NOs:109-129, and a mature light chain variable region having an amino acid sequence at least 95% identical to any one of SEQ ID NOs:23-29, SEQ ID NOs:61-108, and SEQ ID NOs:130-171. Some antibodies comprise a mature heavy chain variable region having an amino acid sequence at least 98% identical to any one of SEQ ID NOs:15-22 and SEQ ID NOs:109-129, and a mature light chain variable region having an amino acid sequence at least 98% identical to any one of SEQ ID NOs:23-29, SEQ ID NOs:61-108, and SEQ ID NOs:130-171. In some antibodies, the mature heavy chain variable region has an amino acid sequence of any one of SEQ ID NOs:15-22 and SEQ ID NOs:109-129, and the mature light chain variable region has an amino acid sequence of any one of SEQ ID NO:23-29, SEQ ID NOs:61-108, and SEQ ID NOs:130-171.

In some antibodies, the mature heavy chain variable region has an amino acid sequence of SEQ ID NO:15 and the mature light chain variable region has an amino acid sequence of SEQ ID NO:23. In some antibodies, the mature heavy chain variable region has an amino acid sequence of SEQ ID NO:15 and the mature light chain variable region has an amino acid sequence of SEQ ID NO:24. In some antibodies, the mature heavy chain variable region has an amino acid sequence of SEQ ID NO:15 and the mature light chain variable region has an amino acid sequence of SEQ ID NO:25. In some antibodies, the mature heavy chain variable region has an amino acid sequence of SEQ ID NO:15 and the mature light chain variable region has an amino acid sequence of SEQ ID NO:26. In some antibodies, the mature heavy chain variable region has an amino acid sequence of SEQ ID NO:15 and the mature light chain variable region has an amino acid sequence of SEQ ID NO:27. In some antibodies, the mature heavy chain variable region has an amino acid sequence of SEQ ID NO:15 and the mature light chain variable region has an amino acid sequence of SEQ ID NO:28. In some antibodies, the mature heavy chain variable region has an amino acid sequence of SEQ ID NO:15 and the mature light chain variable region has an amino acid sequence of SEQ ID NO:29.

In some antibodies, the mature heavy chain variable region has an amino acid sequence of SEQ ID NO:16 and the mature light chain variable region has an amino acid sequence of SEQ ID NO:23. In some antibodies, the mature heavy chain variable region has an amino acid sequence of SEQ ID NO:16 and the mature light chain variable region has an amino acid sequence of SEQ ID NO:24. In some antibodies, the mature heavy chain variable region has an amino acid sequence of SEQ ID NO:16 and the mature light chain variable region has an amino acid sequence of SEQ ID NO:25. In some antibodies, the mature heavy chain variable region has an amino acid sequence of SEQ ID NO:16 and the mature light chain variable region has an amino acid sequence of SEQ ID NO:26. In some antibodies, the mature heavy chain variable region has an amino acid sequence of SEQ ID NO:16 and the mature light chain variable region has an amino acid sequence of SEQ ID NO:27. In some antibodies, the mature heavy chain variable region has an amino acid sequence of SEQ ID NO:16 and the mature light chain variable region has an amino acid sequence of SEQ ID NO:28. In some antibodies, the mature heavy chain variable region has an amino acid sequence of SEQ ID NO:16 and the mature light chain variable region has an amino acid sequence of SEQ ID NO:29.

In some antibodies, the mature heavy chain variable region has an amino acid sequence of SEQ ID NO:17 and the mature light chain variable region has an amino acid sequence of SEQ ID NO:23. In some antibodies, the mature heavy chain variable region has an amino acid sequence of SEQ ID NO:17 and the mature light chain variable region has an amino acid sequence of SEQ ID NO:24. In some antibodies, the mature heavy chain variable region has an amino acid sequence of SEQ ID NO:17 and the mature light chain variable region has an amino acid sequence of SEQ ID NO:25. In some antibodies, the mature heavy chain variable region has an amino acid sequence of SEQ ID NO:17 and the mature light chain variable region has an amino acid sequence of SEQ ID NO:26. In some antibodies, the mature heavy chain variable region has an amino acid sequence of SEQ ID NO:17 and the mature light chain variable region has an amino acid sequence of SEQ ID NO:27. In some antibodies, the mature heavy chain variable region has an amino acid sequence of SEQ ID NO:17 and the mature light chain variable region has an amino acid sequence of SEQ ID NO:28. In some antibodies, the mature heavy chain variable region has an amino acid sequence of SEQ ID NO:17 and the mature light chain variable region has an amino acid sequence of SEQ ID NO:29.

In some antibodies, the mature heavy chain variable region has an amino acid sequence of SEQ ID NO:18 and the mature light chain variable region has an amino acid sequence of SEQ ID NO:23. In some antibodies, the mature heavy chain variable region has an amino acid sequence of SEQ ID NO:18 and the mature light chain variable region has an amino acid sequence of SEQ ID NO:24. In some antibodies, the mature heavy chain variable region has an amino acid sequence of SEQ ID NO:18 and the mature light chain variable region has an amino acid sequence of SEQ ID NO:25. In some antibodies, the mature heavy chain variable region has an amino acid sequence of SEQ ID NO:18 and the mature light chain variable region has an amino acid sequence of SEQ ID NO:26. In some antibodies, the mature heavy chain variable region has an amino acid sequence of SEQ ID NO:18 and the mature light chain variable region has an amino acid sequence of SEQ ID NO:27. In some antibodies, the mature heavy chain variable region has an amino acid sequence of SEQ ID NO:18 and the mature light chain variable region has an amino acid sequence of SEQ ID NO:28. In some antibodies, the mature heavy chain variable region has an amino acid sequence of SEQ ID NO:18 and the mature light chain variable region has an amino acid sequence of SEQ ID NO:29.

In some antibodies, the mature heavy chain variable region has an amino acid sequence of SEQ ID NO:19 and the mature light chain variable region has an amino acid sequence of SEQ ID NO:23. In some antibodies, the mature heavy chain variable region has an amino acid sequence of SEQ ID NO:19 and the mature light chain variable region has an amino acid sequence of SEQ ID NO:24. In some antibodies, the mature heavy chain variable region has an amino acid sequence of SEQ ID NO:19 and the mature light chain variable region has an amino acid sequence of SEQ ID NO:25. In some antibodies, the mature heavy chain variable region has an amino acid sequence of SEQ ID NO:19 and the mature light chain variable region has an amino acid sequence of SEQ ID NO:26. In some antibodies, the mature heavy chain variable region has an amino acid sequence of SEQ ID NO:19 and the mature light chain variable region has an amino acid sequence of SEQ ID NO:27. In some antibodies, the mature heavy chain variable region has an amino acid sequence of SEQ ID NO:19 and the mature light chain variable region has an amino acid sequence of SEQ ID NO:28. In some antibodies, the mature heavy chain variable region has an amino acid sequence of SEQ ID NO:19 and the mature light chain variable region has an amino acid sequence of SEQ ID NO:29.

In some antibodies, the mature heavy chain variable region has an amino acid sequence of SEQ ID NO:20 and the mature light chain variable region has an amino acid sequence of SEQ ID NO:23. In some antibodies, the mature heavy chain variable region has an amino acid sequence of SEQ ID NO:20 and the mature light chain variable region has an amino acid sequence of SEQ ID NO:24. In some antibodies, the mature heavy chain variable region has an amino acid sequence of SEQ ID NO:20 and the mature light chain variable region has an amino acid sequence of SEQ ID NO:25. In some antibodies, the mature heavy chain variable region has an amino acid sequence of SEQ ID NO:20 and the mature light chain variable region has an amino acid sequence of SEQ ID NO:26. In some antibodies, the mature heavy chain variable region has an amino acid sequence of SEQ ID NO:20 and the mature light chain variable region has an amino acid sequence of SEQ ID NO:27. In some antibodies, the mature heavy chain variable region has an amino acid sequence of SEQ ID NO:20 and the mature light chain variable region has an amino acid sequence of SEQ ID NO:28. In some antibodies, the mature heavy chain variable region has an amino acid sequence of SEQ ID NO:20 and the mature light chain variable region has an amino acid sequence of SEQ ID NO:29.

In some antibodies, the mature heavy chain variable region has an amino acid sequence of SEQ ID NO:21 and the mature light chain variable region has an amino acid sequence of SEQ ID NO:23. In some antibodies, the mature heavy chain variable region has an amino acid sequence of SEQ ID NO:21 and the mature light chain variable region has an amino acid sequence of SEQ ID NO:24. In some antibodies, the mature heavy chain variable region has an amino acid sequence of SEQ ID NO:21 and the mature light chain variable region has an amino acid sequence of SEQ ID NO:25. In some antibodies, the mature heavy chain variable region has an amino acid sequence of SEQ ID NO:21 and the mature light chain variable region has an amino acid sequence of SEQ ID NO:26. In some antibodies, the mature heavy chain variable region has an amino acid sequence of SEQ ID NO:21 and the mature light chain variable region has an amino acid sequence of SEQ ID NO:27. In some antibodies, the mature heavy chain variable region has an amino acid sequence of SEQ ID NO:21 and the mature light chain variable region has an amino acid sequence of SEQ ID NO:28. In some antibodies, the mature heavy chain variable region has an amino acid sequence of SEQ ID NO:21 and the mature light chain variable region has an amino acid sequence of SEQ ID NO:29.

In some antibodies, the mature heavy chain variable region has an amino acid sequence of SEQ ID NO:22 and the mature light chain variable region has an amino acid sequence of SEQ ID NO:23. In some antibodies, the mature heavy chain variable region has an amino acid sequence of SEQ ID NO:22 and the mature light chain variable region has an amino acid sequence of SEQ ID NO:24. In some antibodies, the mature heavy chain variable region has an amino acid sequence of SEQ ID NO:22 and the mature light chain variable region has an amino acid sequence of SEQ ID NO:25. In some antibodies, the mature heavy chain variable region has an amino acid sequence of SEQ ID NO:22 and the mature light chain variable region has an amino acid sequence of SEQ ID NO:26. In some antibodies, the mature heavy chain variable region has an amino acid sequence of SEQ ID NO:22 and the mature light chain variable region has an amino acid sequence of SEQ ID NO:27. In some antibodies, the mature heavy chain variable region has an amino acid sequence of SEQ ID NO:22 and the mature light chain variable region has an amino acid sequence of SEQ ID NO:28. In some antibodies, the mature heavy chain variable region has an amino acid sequence of SEQ ID NO:22 and the mature light chain variable region has an amino acid sequence of SEQ ID NO:29.

In some antibodies, the mature heavy chain variable region has an amino acid sequence of SEQ ID NO:127 and the mature light chain variable region has an amino acid sequence of SEQ ID NO: 149. In some antibodies, the mature heavy chain variable region has an amino acid sequence of SEQ ID NO:127 and the mature light chain variable region has an amino acid sequence of SEQ ID NO:142. In some antibodies, the mature heavy chain variable region has an amino acid sequence of SEQ ID NO:127 and the mature light chain variable region has an amino acid sequence of SEQ ID NO:159. In some antibodies, the mature heavy chain variable region has an amino acid sequence of SEQ ID NO:127 and the mature light chain variable region has an amino acid sequence of SEQ ID NO:148.

In some antibodies, the mature heavy chain variable region has an amino acid sequence of SEQ ID NO:127 and the mature light chain variable region has an amino acid sequence of SEQ ID NO:137. In some antibodies, the mature heavy chain variable region has an amino acid sequence of SEQ ID NO:127 and the mature light chain variable region has an amino acid sequence of SEQ ID NO:145. In some antibodies, the mature heavy chain variable region has an amino acid sequence of SEQ ID NO:127 and the mature light chain variable region has an amino acid sequence of SEQ ID NO:136. In some antibodies, the mature heavy chain variable region has an amino acid sequence of SEQ ID NO:127 and the mature light chain variable region has an amino acid sequence of SEQ ID NO:138.

In some antibodies, the mature heavy chain variable region has an amino acid sequence of SEQ ID NO:127 and the mature light chain variable region has an amino acid sequence of SEQ ID NO:158. In some antibodies, the mature heavy chain variable region has an amino acid sequence of SEQ ID NO:127 and the mature light chain variable region has an amino acid sequence of SEQ ID NO:143. In some antibodies, the mature heavy chain variable region has an amino acid sequence of SEQ ID NO:127 and the mature light chain variable region has an amino acid sequence of SEQ ID NO:144. In some antibodies, the mature heavy chain variable region has an amino acid sequence of SEQ ID NO:127 and the mature light chain variable region has an amino acid sequence of SEQ ID NO:133.

In some antibodies, the mature heavy chain variable region has an amino acid sequence of SEQ ID NO:127 and the mature light chain variable region has an amino acid sequence of SEQ ID NO:160. In some antibodies, the mature heavy chain variable region has an amino acid sequence of SEQ ID NO:127 and the mature light chain variable region has an amino acid sequence of SEQ ID NO:161. In some antibodies, the mature heavy chain variable region has an amino acid sequence of SEQ ID NO:127 and the mature light chain variable region has an amino acid sequence of SEQ ID NO:139. In some antibodies, the mature heavy chain variable region has an amino acid sequence of SEQ ID NO:128 and the mature light chain variable region has an amino acid sequence of SEQ ID NO:168.

Some such antibodies comprise three light chain CDRs and three heavy chain CDRs of monoclonal antibody 10C12, wherein 10C12 is a mouse antibody characterized by a heavy chain variable region having an amino acid sequence comprising SEQ ID NO:7 and a light chain variable region having an amino acid sequence comprising SEQ ID NO:11.

In some such antibodies, the three heavy chain CDRs CDR-H1, CDR-H2, and CDR-H3 are as defined by Kabat/Chothia Composite (SEQ ID NOs:8, 9, and 10, respectively), and the three light chain CDRs CDR-L1, CDR-L2, and CDR-L3 are as defined by Kabat/Chothia Composite (SEQ ID NOs:12, 13, and 14, respectively.

Some antibodies are 10C12 or a chimeric, veneered, or humanized form thereof. In some antibodies, the variable heavy chain has ≥85% identity to human sequence. In some antibodies, the variable light chain has ≥85% identity to human sequence. In some antibodies, each of the variable heavy chain and variable light chain has ≥85% identity to human germline sequence. Some antibodies are humanized antibodies.

Some antibodies are a humanized or chimeric 10C12 antibody that specifically binds to human tau, wherein 10C12 is a mouse antibody characterized by a mature heavy chain variable region of SEQ ID NO:7 and a mature light chain variable region of SEQ ID NO:11. Some antibodies comprise a humanized mature heavy chain variable region comprising the three heavy chain CDRs of 10C12 and a humanized mature light chain variable region comprising the three light chain CDRs of 10C12. In some antibodies, the CDRs are of a definition selected from the group of Kabat, Chothia, Kabat/Chothia Composite, AbM and Contact.

In some antibodies, the humanized mature heavy chain variable region comprises the three Kabat/Chothia Composite heavy chain CDRs of 10C12 (SEQ ID NOs:8-10) and the humanized mature light chain variable region comprises the three Kabat/Chothia Composite light chain CDRs of 10C12 (SEQ ID NOs:12-14). In some antibodies, the humanized mature heavy chain variable region comprises the three Kabat heavy chain CDRs of 10C12 (SEQ ID NO:40, SEQ ID NO:9, and SEQ ID NO:10) and the humanized mature light chain variable region comprises the three Kabat light chain CDRs of 10C12 (SEQ ID NOs:12-14). In some antibodies, the humanized mature heavy chain variable region comprises the three Chothia heavy chain CDRs of 10C12 (SEQ ID NO:41, SEQ ID NO:42, and SEQ ID NO:10) and the humanized mature light chain variable region comprises the three Chothia light chain CDRs of 10C12 (SEQ ID NOs:12-14). In some antibodies, the humanized mature heavy chain variable region comprises the three AbM heavy chain CDRs of 10C12 (SEQ ID NO:8, SEQ ID NO:43, and SEQ ID NO:10) and the humanized mature light chain variable region comprises the three AbM light chain CDRs of 10C12 (SEQ ID NOs:12-14). In some antibodies, the humanized mature heavy chain variable region comprises the three Contact heavy chain CDRs of 10C12 (SEQ ID NO:44-46) and the humanized mature light chain variable region comprises the three Contact light chain CDRs of 10C12 (SEQ ID NO:47-49).

For example, the antibody can a humanized antibody, veneered antibody, or chimeric antibody.

Some such antibodies comprise a humanized mature heavy chain variable region having an amino acid sequence at least 90% identical to any one of SEQ ID NOs:214-215 and a humanized mature light chain variable region having an amino acid sequence at least 90% identical to any one of SEQ ID NOs:216-217.

In some antibodies, at least one of the following positions in the VH region is occupied by the amino acid as specified: H24 is occupied by A, H48 is occupied by I, H67 is occupied by A, H69 is occupied by M, H93 is occupied by T, and H94 is occupied by T. In some antibodies, positions H24, H48, H67, H69, H93, and H94 are occupied by A, I, A, M, T, and T, respectively.

In some antibodies, at least one of the following positions in the VH region is occupied by the amino acid as specified: H1 is occupied by Q or E, H24 is occupied by A, H48 is occupied by I, H67 is occupied by A, H69 is occupied by M, H93 is occupied by T, H94 is occupied by T.

In some antibodies, positions H24, H48, H67, H69, H93, and H94 are occupied by A, I, A, M, T, and T, respectively. In some antibodies, positions H1, H24, H48, H67, H69, H93, and H94 are occupied by E, A, I, A, M, T, and T, respectively.

In some antibodies, position L64 in the VL region is occupied by S.

In some antibodies, at least one of the following positions in the VL region is occupied by the amino acid as specified: L64 is S, L104 is V or L. In some antibodies, position L64 is occupied by S. In some antibodies, positions L64 and L104 in the VL region are occupied by S and L, respectively.

Some antibodies comprise a mature heavy chain variable region having an amino acid sequence at least 95% identical to any one of SEQ ID NOs:214-215 and a mature light chain variable region having an amino acid sequence at least 95% identical to any one of SEQ ID NOs:216-217. Some antibodies comprise a a mature heavy chain variable region having an amino acid sequence at least 98% identical to any one of SEQ ID NOs:214-215 and a mature light chain variable region having an amino acid sequence at least 98% identical to any one of SEQ ID NOs:216-217. In some antibodies, the mature heavy chain variable region has an amino acid sequence of any one of SEQ ID NOs:214-215 and the mature light chain variable region has an amino acid sequence of any one of SEQ ID NO:216-217.

In some antibodies, the mature heavy chain variable region has an amino acid sequence of SEQ ID NO:214 and the mature light chain variable region has an amino acid sequence of SEQ ID NO:216. In some antibodies, the mature heavy chain variable region has an amino acid sequence of SEQ ID NO:214 and the mature light chain variable region has an amino acid sequence of SEQ ID NO:217. In some antibodies, the mature heavy chain variable region has an amino acid sequence of SEQ ID NO:215 and the mature light chain variable region has an amino acid sequence of SEQ ID NO:216. In some antibodies, the mature heavy chain variable region has an amino acid sequence of SEQ ID NO:215 and the mature light chain variable region has an amino acid sequence of SEQ ID NO:217.

Some such antibodies comprise three light chain CDRs and three heavy chain CDRs of monoclonal antibody 12C4, wherein 12C4 is a mouse antibody characterized by a heavy chain variable region having an amino acid sequence comprising SEQ ID NO:219 and a light chain variable region having an amino acid sequence comprising SEQ ID NO:11.

In some such antibodies, the three heavy chain CDRs CDR-H1, CDR-H2, and CDR-H3 are as defined by Kabat/Chothia Composite (SEQ ID NOs:8, 220, and 10, respectively), and the three light chain CDRs CDR-L1, CDR-L2, and CDR-L3 are as defined by Kabat/Chothia Composite (SEQ ID NOs:12, 13, and 14, respectively).

Some antibodies are 12C4 or a chimeric, veneered, or humanized form thereof. In some antibodies, the variable heavy chain has ≥85% identity to human sequence. In some antibodies, the variable light chain has ≥85% identity to human sequence. In some antibodies, each of the variable heavy chain and variable light chain has ≥85% identity to human germline sequence. Some antibodies are humanized antibodies.

Some antibodies are a humanized or chimeric 12C4 antibody that specifically binds to human tau, wherein 12C4 is a mouse antibody characterized by a mature heavy chain variable region of SEQ ID NO:219 and a mature light chain variable region of SEQ ID NO:11. Some antibodies comprise a humanized mature heavy chain variable region comprising the three heavy chain CDRs of 12C4 and a humanized mature light chain variable region comprising the three light chain CDRs of 12C4. In some antibodies, the CDRs are of a definition selected from the group of Kabat, Chothia, Kabat/Chothia Composite, AbM and Contact.

In some antibodies, the humanized mature heavy chain variable region comprises the three Kabat/Chothia Composite heavy chain CDRs of 12C4 (SEQ ID NOs:8, 220, and 10) and the humanized mature light chain variable region comprises the three Kabat/Chothia Composite light chain CDRs of 12C4 (SEQ ID NOs:12-14). In some antibodies, the humanized mature heavy chain variable region comprises the three Kabat heavy chain CDRs of 12C4 (SEQ ID NO:40, SEQ ID NO:220, and SEQ ID NO:10) and the humanized mature light chain variable region comprises the three Kabat light chain CDRs of 12C4 (SEQ ID NOs:12-14). In some antibodies, the humanized mature heavy chain variable region comprises the three Chothia heavy chain CDRs of 12C4 (SEQ ID NO:41, SEQ ID NO:42, and SEQ ID NO:10) and the humanized mature light chain variable region comprises the three Chothia light chain CDRs of 12C4 (SEQ ID NOs:12-14). In some antibodies, the humanized mature heavy chain variable region comprises the three AbM heavy chain CDRs of 12C4 (SEQ ID NO:8, SEQ ID NO:257, and SEQ ID NO:10) and the humanized mature light chain variable region comprises the three AbM light chain CDRs of 12C4 (SEQ ID NOs:12-14). In some antibodies, the humanized mature heavy chain variable region comprises the three Contact heavy chain CDRs of 12C4 (SEQ ID NO:44, 258, and 46) and the humanized mature light chain variable region comprises the three Contact light chain CDRs of 12C4 (SEQ ID NO:47-49).

For example, the antibody can be a humanized antibody, veneered antibody, or chimeric antibody.

Some such antibodies comprise a humanized mature heavy chain variable region having an amino acid sequence at least 90% identical to any one of SEQ ID NOs:221-222 and a humanized mature light chain variable region having an amino acid sequence at least 90% identical to any one of SEQ ID NOs:223-224.

In some antibodies, at least one of the following positions in the VH region is occupied by the amino acid as specified: H1 is occupied by Q or E, H48 is occupied by M or I, H93 is occupied by A or T, H94 is occupied by R or T. In some antibodies, positions H1, H48, H93, and H94 in the VH region are occupied by E, I, T, and T, respectively.

In some antibodies, positions in the VL region is occupied by the amino acid as specified: L64 is G or S, L104 is V or L. In some antibodies, positions L64 and L104 in the VL region are occupied by S and L, respectively.

Some antibodies comprise a mature heavy chain variable region having an amino acid sequence at least 95% identical to any one of SEQ ID NOs:221-222 and a mature light chain variable region having an amino acid sequence at least 95% identical to any one of SEQ ID NOs:223-224. Some antibodies comprise a mature heavy chain variable region having an amino acid sequence at least 98% identical to any one of SEQ ID NOs:221-222 and a mature light chain variable region having an amino acid sequence at least 98% identical to any one of SEQ ID NOs:223-224. In some antibodies, the mature heavy chain variable region has an amino acid sequence of any one of SEQ ID NOs:221-222 and the mature light chain variable region has an amino acid sequence of any one of SEQ ID NO:223-224.

In some antibodies, the mature heavy chain variable region has an amino acid sequence of SEQ ID NO:221 and the mature light chain variable region has an amino acid sequence of SEQ ID NO:223. In some antibodies, the mature heavy chain variable region has an amino acid sequence of SEQ ID NO:221 and the mature light chain variable region has an amino acid sequence of SEQ ID NO:224. In some antibodies, the mature heavy chain variable region has an amino acid sequence of SEQ ID NO:222 and the mature light chain variable region has an amino acid sequence of SEQ ID NO:223. In some antibodies, the mature heavy chain variable region has an amino acid sequence of SEQ ID NO:222 and the mature light chain variable region has an amino acid sequence of SEQ ID NO:224

Some such antibodies comprise three light chain CDRs and three heavy chain CDRs of monoclonal antibody 17C12, wherein 17C12 is a mouse antibody characterized by a heavy chain variable region having an amino acid sequence comprising SEQ ID NO:225 and a light chain variable region having an amino acid sequence comprising SEQ ID NO:228.

In some such antibodies, the three heavy chain CDRs CDR-H1, CDR-H2, and CDR-H3 are as defined by Kabat/Chothia Composite (SEQ ID NOs:226, 227, and 10, respectively), and the three light chain CDRs CDR-L1, CDR-L2, and CDR-L3 are as defined by Kabat/Chothia Composite (SEQ ID NOs:229, 230, and 231, respectively).

Some antibodies are 17C12 or a chimeric, veneered, or humanized form thereof. In some antibodies, the variable heavy chain has ≥85% identity to human sequence. In some antibodies, the variable light chain has ≥85% identity to human sequence. In some antibodies, each of the variable heavy chain and variable light chain has ≥85% identity to human germline sequence. Some antibodies are humanized antibodies.

Some antibodies are a humanized or chimeric 17C12 antibody that specifically binds to human tau, wherein 17C12 is a mouse antibody characterized by a mature heavy chain variable region of SEQ ID NO:225 and a mature light chain variable region of SEQ ID NO:228. Some antibodies comprise a humanized mature heavy chain variable region comprising the three heavy chain CDRs of 17C12 and a humanized mature light chain variable region comprising the three light chain CDRs of 17C12. In some antibodies, the CDRs are of a definition selected from the group of Kabat, Chothia, Kabat/Chothia Composite, AbM and Contact.

In some antibodies, the humanized mature heavy chain variable region comprises the three Kabat/Chothia Composite heavy chain CDRs of 17C12 (SEQ ID NOs:226, 227, and 10) and the humanized mature light chain variable region comprises the three Kabat/Chothia Composite light chain CDRs of 17C12 (SEQ ID NOs:229-231). In some antibodies, the humanized mature heavy chain variable region comprises the three Kabat heavy chain CDRs of 17C12 (SEQ ID NO:40, SEQ ID NO:227, and SEQ ID NO:10) and the humanized mature light chain variable region comprises the three Kabat light chain CDRs of 17C12 (SEQ ID NOs:229-231). In some antibodies, the humanized mature heavy chain variable region comprises the three Chothia heavy chain CDRs of 17C12 (SEQ ID NO:259, SEQ ID NO:42, and SEQ ID NO:10) and the humanized mature light chain variable region comprises the three Chothia light chain CDRs of 17C12 (SEQ ID NOs:229-231). In some antibodies, the humanized mature heavy chain variable region comprises the three AbM heavy chain CDRs of 17C12 (SEQ ID NO:226, SEQ ID NO:260, and SEQ ID NO:10) and the humanized mature light chain variable region comprises the three AbM light chain CDRs of 17C12 (SEQ ID NOs:229-231). In some antibodies, the humanized mature heavy chain variable region comprises the three Contact heavy chain CDRs of 17C12 (SEQ ID NO:44, SEQ ID NO:261, and SEQ ID NO:46) and the humanized mature light chain variable region comprises the three Contact light chain CDRs of 17C12 (SEQ ID NO:262-264).

For example, the antibody can be is a humanized antibody, veneered antibody, or chimeric antibody.

Some such antibodies comprise a humanized mature heavy chain variable region having an amino acid sequence at least 90% identical to any one of SEQ ID NOs:232-233 and a humanized mature light chain variable region having an amino acid sequence at least 90% identical to any one of SEQ ID NOs:234-235.

In some antibodies, at least one of the following positions in the VH region is occupied by the amino acid as specified: H2 is occupied by I, H24 is occupied by A, H48 is occupied by I, H67 is occupied by A, H69 is occupied by M, H93 is occupied by T, and H94 is occupied by T. In some antibodies, positions H2, H24, H48, H67, H69, H93, and H94 are occupied by E, A, I, A, M, T, and T, respectively.

In some antibodies, at least one of the following positions in the VH region is occupied by the amino acid as specified: H1 is occupied by Q or E, H2 is occupied by I, H24 is occupied by A, H48 is occupied by I, H67 is occupied by A, H69 is occupied by M, H93 is occupied by T, H94 is occupied by T, H108 is occupied by T or L, H113 is occupied by R or S. In some antibodies, positions H2, H24, H48, H67, H69, H93, and H94 in the VH region are occupied by E, A, I, A, M, T, and T , respectively. In some antibodies, positions H1, H2, H24, H48, H67, H69, H93, H94, H108, and H113 in the VH region are occupied by E, I, A, I, A, M, T. T. L, and S, respectively.

In some antibodies, at least one of the following positions in the VL region is occupied by the amino acid as specified: L2 is occupied by V, and L36 is occupied by L. In some antibodies, positions L2 and L36 are occupied by V and L, respectively.

In some antibodies, at least one of the following positions in the VL region is occupied by the amino acid as specified: L2 is V, L36 is L, L43 is P or S. In some antibodies, positions L2 and L36 in the VL region are occupied by V and L, respectively. In some antibodies, positions L2, L36, and L43 in the VL region are occupied by V, L, and S, respectively.

Some antibodies comprise a mature heavy chain variable region having an amino acid sequence at least 95% identical to any one of SEQ ID NOs:232-233 and a mature light chain variable region having an amino acid sequence at least 95% identical to any one of SEQ ID NOs:234-235. Some antibodies comprise a mature heavy chain variable region having an amino acid sequence at least 98% identical to any one of SEQ ID NOs:232-233 and a mature light chain variable region having an amino acid sequence at least 98% identical to any one of SEQ ID NOs:234-235. In some antibodies, the mature heavy chain variable region has an amino acid sequence of any one of SEQ ID NOs:232-233and the mature light chain variable region has an amino acid sequence of any one of SEQ ID NO:234-235.

In some antibodies, the mature heavy chain variable region has an amino acid sequence of SEQ ID NO:232 and the mature light chain variable region has an amino acid sequence of SEQ ID NO:234. In some antibodies, the mature heavy chain variable region has an amino acid sequence of SEQ ID NO:232 and the mature light chain variable region has an amino acid sequence of SEQ ID NO:235. In some antibodies, the mature heavy chain variable region has an amino acid sequence of SEQ ID NO:233 and the mature light chain variable region has an amino acid sequence of SEQ ID NO:234. In some antibodies, the mature heavy chain variable region has an amino acid sequence of SEQ ID NO:233 and the mature light chain variable region has an amino acid sequence of SEQ ID NO:235.

Some such antibodies comprise three light chain CDRs and three heavy chain CDRs of monoclonal antibody 14H3, wherein 14H3 is a mouse antibody characterized by a heavy chain variable region having an amino acid sequence comprising SEQ ID NO:240 and a light chain variable region having an amino acid sequence comprising SEQ ID NO:244.

In some such antibodies, the three heavy chain CDRs CDR-H1, CDR-H2, and CDR-H3 are as defined by Kabat/Chothia Composite (SEQ ID NOs:241, 242, and 243, respectively), except that position H35B can be occupied by G or S, and the three light chain CDRs CDR-L1, CDR-L2, and CDR-L3 are as defined by Kabat/Chothia Composite (SEQ ID NOs:245, 246, and 247, respectively). In some antibodies, CDR-H1 has an amino acid sequence comprising SEQ ID NO:277.

Some antibodies are 14H3 or a chimeric, veneered, or humanized form thereof. In some antibodies, the variable heavy chain has ≥85% identity to human sequence. In some antibodies, the variable light chain has ≥85% identity to human sequence. In some antibodies, each of the variable heavy chain and variable light chain has ≥85% identity to human germline sequence. Some antibodies are humanized antibodies.

Some antibodies are a humanized or chimeric 14H3 antibody that specifically binds to human tau, wherein 14H3 is a mouse antibody characterized by a mature heavy chain variable region of SEQ ID NO:240 and a mature light chain variable region of SEQ ID NO:244. Some antibodies comprise a humanized mature heavy chain variable region comprising the three heavy chain CDRs of 14H3 and a humanized mature light chain variable region comprising the three light chain CDRs of 14H3. In some antibodies, the CDRs are of a definition selected from the group of Kabat, Chothia, Kabat/Chothia Composite, AbM and Contact.

In some antibodies, wherein the humanized mature heavy chain variable region comprises the three Kabat/Chothia Composite heavy chain CDRs of 14H3 (SEQ ID NOs:241-243) and the humanized mature light chain variable region comprises the three Kabat/Chothia Composite light chain CDRs of 14H3 (SEQ ID NOs:245-247). In some antibodies, the humanized mature heavy chain variable region comprises the three Kabat heavy chain CDRs of 14H3 (SEQ ID NO:265, SEQ ID NO:242, and SEQ ID NO:243) and the humanized mature light chain variable region comprises the three Kabat light chain CDRs of 14H3 (SEQ ID NOs:245-247). In some antibodies, the humanized mature heavy chain variable region comprises the three Chothia heavy chain CDRs of 14H3 (SEQ ID NO:266, SEQ ID NO:267, and SEQ ID NO:243) and the humanized mature light chain variable region comprises the three Chothia light chain CDRs of 14H3 (SEQ ID NOs:245-247). In some antibodies, the humanized mature heavy chain variable region comprises the three AbM heavy chain CDRs of 14H3 (SEQ ID NO:241, SEQ ID NO:268, and SEQ ID NO:243) and the humanized mature light chain variable region comprises the three AbM light chain CDRs of 14H3 (SEQ ID NOs:245-247). In some antibodies, the humanized mature heavy chain variable region comprises the three Contact heavy chain CDRs of 14H3 (SEQ ID NO:269-271) and the humanized mature light chain variable region comprises the three Contact light chain CDRs of 14H3 (SEQ ID NO:272-274).

For example, the antibody can be a humanized antibody, veneered antibody, or chimeric antibody.

Some such antibodies comprise a humanized mature heavy chain variable region having an amino acid sequence at least 90% identical to any one of SEQ ID NOs:248-249 and a humanized mature light chain variable region having an amino acid sequence at least 90% identical to any one of SEQ ID NOs:250-251.

In some antibodies, position H35B in the VH region is occupied by S.

In some antibodies, at least one of the following positions in the VH region is occupied by the amino acid as specified: H35B is occupied by S, H108 is occupied by M or L, H113 is occupied by L or S. In some antibodies, position H35B in the VH region is occupied by S. In some antibodies, positions H35B, H108, and H113 in the VH region are occupied by S, L, and S, respectively.

In some antibodies, at least one of the following positions in the VL region is occupied by the amino acid as specified: L2 is occupied by V and L87 is occupied by F. In some antibodies, positions L2 and L87 are occupied by V and F, respectively.

In some antibodies, at least one of the following positions in the VL region is occupied by the amino acid as specified: L2 is V, L7 is T or S, L37 is L or Q, L87 is F, L100 is G or Q, L104 is V or L. In some antibodies, positions L2 and L87 in the VL region are occupied by V and F, respectively. In some antibodies, positions L2, L7, L37, L87, L100, and L104 in the VL region are occupied by V, S, Q, F, Q, and L, respectively.

Some antibodies comprise a mature heavy chain variable region having an amino acid sequence at least 95% identical to any one of SEQ ID NOs:248-249 and a mature light chain variable region having an amino acid sequence at least 95% identical to any one of SEQ ID NOs:250-251. Some antibodies comprise a mature heavy chain variable region having an amino acid sequence at least 98% identical to any one of SEQ ID NOs:248-249 and a mature light chain variable region having an amino acid sequence at least 98% identical to any one of SEQ ID NOs:250-251. In some antibodies, the mature heavy chain variable region has an amino acid sequence of any one of SEQ ID NOs:248-249 and the mature light chain variable region has an amino acid sequence of any one of SEQ ID NO:250-251

In some antibodies, the mature heavy chain variable region has an amino acid sequence of SEQ ID NO:248 and the mature light chain variable region has an amino acid sequence of SEQ ID NO:250. In some antibodies, the mature heavy chain variable region has an amino acid sequence of SEQ ID NO:248 and the mature light chain variable region has an amino acid sequence of SEQ ID NO:251. In some antibodies, the mature heavy chain variable region has an amino acid sequence of SEQ ID NO:249 and the mature light chain variable region has an amino acid sequence of SEQ ID NO:250. In some antibodies, the mature heavy chain variable region has an amino acid sequence of SEQ ID NO:249 and the mature light chain variable region has an amino acid sequence of SEQ ID NO:251.

For example, the antibody can be a chimeric antibody. For example, the antibody can be a veneered antibody.

The antibody can be an intact antibody. The antibody can be a binding fragment. In an embodiment, the binding fragment is a single-chain antibody, Fab, or Fab′2 fragment. The antibody can be a Fab fragment, or single chain Fv. Some of the antibodies have a human IgG1 isotype, while others may have a human IgG2 or IgG4 isotype. Some antibodies have the mature light chain variable region fused to a light chain constant region and the mature heavy chain variable region fused to a heavy chain constant region. The heavy chain constant region of some antibodies is a mutant form of a natural human heavy chain constant region which has reduced binding to a Fcγ receptor relative to the natural human heavy chain constant region. In some antibodies, the heavy chain constant region is of IgG1 isotype.

Some antibodies may have at least one mutation in the constant region, such as a mutation that reduces complement fixation or activation by the constant region, for example a mutation at one or more of positions 241, 264, 265, 270, 296, 297, 318, 320, 322, 329 and 331 by EU numbering. Some antibodies have an alanine at positions 318, 320 and 322. Some antibodies can be at least 95% w/w pure. The antibody can be conjugated to a therapeutic, cytotoxic, cytostatic, neurotrophic, or neuroprotective agent.

In another aspect, the invention provides a pharmaceutical composition comprising any of the antibodies disclosed herein and a pharmaceutically-acceptable carrier.

In another aspect, the invention provides a nucleic acid encoding the heavy chain and/or light chain of any of the antibodies disclosed herein, a recombinant expression vector comprising the nucleic acid and a host cell transformed with the recombinant expression vector. Some nucleic acids have a sequence comprising any one of SEQ ID NOs:38-39.

In another aspect, the invention provides a vector comprising a nucleic acid encoding a mature heavy chain variable region and a mature light chain variable region operably linked to one or more regulatory sequences to effect expression in a mammalian cell of any of the antibodies disclosed herein, a recombinant expression vector comprising the nucleic acid, a host cell transformed with the recombinant expression vector, and a host cell transformed with the nucleic acid. Some nucleic acids further encode a heavy chain constant region fused to the mature heavy chain variable region and a light chain constant region fused to the mature light chain variable region. In some vectors, the antibody is a scFv. In some vectors, the antibody is a Fab fragment. In some vectors, the one or more regulatory sequences include one or more of a promoter, enhancer, ribosome binding site, and transcription termination signal. In some vectors, the nucleic acid further encodes signal peptides fused to the mature heavy and light chain variable regions. In some vectors, the nucleic acid is codon-optimized for expression in a host cell. In some vectors, the one or more regulatory sequences include a eukaryotic promoter. In some vectors, the nucleic acid further encodes a selectable gene.

In yet another aspect, the invention provides methods of expressing an antibody in a mammalian cell comprising incorporating the nucleic acids disclosed herein into the genome of a transgenic animal, whereby the antibody is expressed.

In yet another aspect, the invention provides first and second vectors respectively comprising nucleic acids encoding a mature heavy chain variable region and a mature light chain variable region, each operably linked to one or more regulatory sequences to effect expression in a mammalian cell of any of the antibodies disclosed herein, and a host cell comprising the nucleic acids. In some first and second vectors, the nucleic acids respectively further encode a heavy chain constant region fused to the mature heavy chain variable region and a light chain constant region fused to the mature light chain variable region.

In yet another aspect, the invention provides methods of expressing an antibody in a mammalian cell comprising incorporating any of the nucleic acids disclosed herein into the genome of a transgenic animal, whereby the antibody is expressed.

In yet another aspect, the invention provides methods of humanizing any non-human antibody described herein, for example, mouse antibody 9F5, wherein 9F5 is characterized by a mature heavy chain variable region of SEQ ID NO:7 and a mature light chain variable region of SEQ ID NO:11, for example, mouse antibody 10C12, wherein 10C12 is characterized by a mature heavy chain variable region of SEQ ID NO:7 and a mature light chain variable region of SEQ ID NO:11; for example, mouse antibody is 2D11, wherein 2D11 is characterized by a mature heavy chain variable region of SEQ ID NO:7 and a mature light chain variable region of SEQ ID NO:11; for example mouse antibody 12C4, wherein 12C4 is characterized by a mature heavy chain variable region of SEQ ID NO:219 and a mature light chain variable region of SEQ ID NO:11; for example mouse antibody 17C12, wherein 17C12 is characterized by a mature heavy chain variable region of SEQ ID NO:225 and a mature light chain variable region of SEQ ID NO:228; for example mouse antibody 14H3, wherein 14H3 is characterized by a mature heavy chain variable region of SEQ ID NO:240 and a mature light chain variable region of SEQ ID NO:244. Such methods can involve selecting one or more acceptor antibodies, identifying the amino acid residues of the mouse antibody to be retained; synthesizing a nucleic acid encoding a humanized heavy chain comprising CDRs of the mouse antibody heavy chain and a nucleic acid encoding a humanized light chain comprising CDRs of the mouse antibody light chain, and expressing the nucleic acids in a host cell to produce a humanized antibody.

Methods of producing antibodies, such as a humanized, chimeric or veneered antibody, for example humanized, chimeric or veneered forms of 9F5, 10C12, 2D11, 12C4, 17C12, or 14H3, are also provided. In such methods, cells transformed with nucleic acids encoding the heavy and light chains of the antibody are cultured so that the cells secrete the antibody. The antibody can then be purified from the cell culture media.

Cell lines producing any of the antibodies disclosed herein can be produced by introducing a vector encoding heavy and light chains of the antibody and a selectable marker into cells, propagating the cells under conditions to select for cells having increased copy number of the vector, isolating single cells from the selected cells; and banking cells cloned from a single cell selected based on yield of antibody.

Some cells can be propagated under selective conditions and screened for cell lines naturally expressing and secreting at least 100 mg/L/10⁶ cells/24 h. Single cells can be isolated from the selected cells. Cells cloned from a single cell can then be banked. Single cells can be selected based on desirable properties, such as the yield of the antibody. Exemplary cell lines are cell lines expressing 9F5, 10C12, 2D11, 12C4, 17C12, or 14H3.

The invention also provides methods of inhibiting or reducing aggregation of tau in a subject having or at risk of developing a tau-mediated amyloidosis, comprising administering to the subject an effective regime of an antibody disclosed herein, thereby inhibiting or reducing aggregation of tau in the subject. Exemplary antibodies include humanized versions of 9F5, 10C12, 2D11, 12C4, 17C12, or 14H3.

Also provided are methods of treating or effecting prophylaxis of a tau-related disease in a subject, comprising administering an effective regime of an antibody disclosed herein and thereby treating or effecting prophylaxis of the disease. Examples of such a disease are Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP). In some methods, the tau-related disease is Alzheimer's disease. In some methods the patient is an ApoE4 carrier.

Also provided are methods of reducing aberrant transmission of tau comprising administering an effective regime of an antibody an antibody disclosed herein and thereby reducing transmission of tau.

Also provided are methods of inducing phagocytosis of tau comprising administering an effective regime of an antibody disclosed herein and thereby inducing phagocytosis of tau.

Also provided are methods of inhibiting tau aggregation or deposition comprising administering an effective regime of an antibody disclosed herein thereby inhibiting tau aggregation or deposition.

Also provided are methods of inhibiting formation of tau tangles comprising administering an effective regime of an antibody disclosed herein.

The invention also provides a method of detecting tau protein deposits in a subject having or at risk of a disease associated with tau aggregation or deposition, comprising administering to a subject an antibody disclosed herein, and detecting the antibody bound to tau in the subject. Examples of such a disease are Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP). In some embodiments, the antibody is administered by intravenous injection into the body of the subject. In some embodiments, the antibody is administered directly to the brain of the subject by intracranial injection or by drilling a hole through the skull of the subject. In some embodiments, the antibody is labeled. In some embodiments, the antibody is labeled with a fluorescent label, a paramagnetic label, or a radioactive label. In some embodiments, the radioactive label is detected using positron emission tomography (PET) or single-photon emission computed tomography (SPECT).

The invention also provides a method of measuring efficacy of treatment in a subject being treated for a disease associated with tau aggregation or deposition, comprising measuring a first level of tau protein deposits in the subject prior to treatment by administering to a subject an antibody disclosed herein, and detecting a first amount of the antibody bound to tau in the subject, administering the treatment to the subject, measuring a second level of tau protein deposits in the subject after treatment by administering to a subject the antibody, and detecting the antibody bound to tau in the subject, wherein a decrease in the level of tau protein deposits indicates a positive response to treatment.

The invention also provides a method of measuring efficacy of treatment in a subject being treated for a disease associated with tau aggregation or deposition, comprising measuring a first level of tau protein deposits in the subject prior to treatment by administering to a subject an antibody disclosed herein, and detecting a first amount of antibody bound to tau in the subject, administering the treatment to the subject, measuring a second level of tau protein deposits in the subject after treatment by administering to a subject the antibody, and detecting a second amount of antibody bound to tau in the subject, wherein no change in the level of tau protein deposits or a small increase in tau protein deposits indicates a positive response to treatment.

The invention also provides an isolated monoclonal antibody that specifically binds to a peptide consisting of residues (Q/E)IVYK(S/P) (SEQ ID NO:56). The invention also provides an isolated monoclonal antibody that specifically binds to a peptide consisting of residues QIVYKP (SEQ ID NO:57). The invention also provides an isolated monoclonal antibody that specifically binds to a peptide consisting of residues EIVYKSP (SEQ ID NO:58). The invention also provides an isolated monoclonal antibody that specifically binds to a peptide consisting of residues EIVYKS (SEQ ID NO:277).

The invention also provides an isolated monoclonal antibody that specifically binds to the polypeptide of SEQ ID NO:1 at an epitope including at least one residue within 307-312 of SEQ ID NO:1. Some such antibodies bind to an epitope within residues 307-312 of SEQ ID NO:1. The invention also provides an isolated monoclonal antibody that specifically binds to the polypeptide of SEQ ID NO:1 at an epitope including at least one residue within residues 391-397 of SEQ ID NO:1. Some such antibodies bind to an epitope within residues 391-397 of SEQ ID NO:1. The invention also provides an isolated monoclonal antibody that specifically binds to the polypeptide of SEQ ID NO:1 at an epitope including at least one residue within residues 391-396 of SEQ ID NO:1. Some such antibodies bind to an epitope within residues 391-396 of SEQ ID NO:1. The invention also provides an isolated monoclonal antibody that specifically binds to the polypeptide of SEQ ID NO:1 at an epitope including at least one residue from within residues 307-312 of SEQ ID NO:1 and at least one residue from within residues 391-397 of SEQ ID NO:1. The invention also provides an isolated monoclonal antibody that specifically binds to the polypeptide of SEQ ID NO:1 at an epitope including at least one residue from within residues 307-312 of SEQ ID NO:1 and at least one residue from within residues 391-396 of SEQ ID NO:1.

The invention also provides a method of treating or effecting prophylaxis of a tau-related disease in a subject comprising administering an immunogen comprising a tau peptide of up to 20 contiguous amino acids of SEQ ID NO:1 to which antibody 9F5, 10C12, 2D11, 12C4, 17C12, or 14H3 specifically binds, wherein the peptide induces formation of antibodies specifically binding to tau in the subject. In some such methods, the tau peptide consists of 4-7 contiguous amino acids from residues 307-312 of SEQ ID NO:1 or from residues 391-397 of SEQ ID NO:1 or from residues 391-396 of SEQ ID NO:1. In some such methods, the tau peptide consists of residues (Q/E)IVYK(S/P) (SEQ ID NO:56). In some such methods, the tau peptide consists of residues QIVYKP (SEQ ID NO:57). In some such methods, the tau peptide consists of residues EIVYKSP (SEQ ID NO:58). In some such methods, the tau peptide consists of residues EIVYKS (SEQ ID NO:277). In some such methods, the tau peptide is attached to a heterologous conjugate molecule.

The invention also provides a method of producing an antibody that specifically binds to an epitope comprising (Q/E)IVYK(S/P) (SEQ ID NO:56), comprising immunizing an animal with tau or a fragment thereof, and screening for antibodies that specifically bind to the epitope. In some such methods, the animal is immunized with 383 amino acid human tau (4R0N). In some such methods, the human tau contains a P301S mutation. In some such methods, the human tau is recombinant N-terminally His-tagged.

In some such methods, the screening is performed against 15 amino acid peptides comprising QIVYKP (SEQ ID NO:57), EIVYKSP (SEQ ID NO:58) EIVYKS (SEQ ID NO:277) or any other consensus motif represented by (Q/E)IVYK(S/P) (SEQ ID NO:56). In some such methods, the peptides comprise QIVYKP (SEQ ID NO:57) or EIVYKSP (SEQ ID NO:58) or EIVYKS (SEQ ID NO:277).

In some such methods, the animal is immunized with a tau fragment comprising a peptide represented by (Q/E)IVYK(S/P) (SEQ ID NO:56), linked to a carrier. In some such methods, the peptide is QIVYKP (SEQ ID NO:57) or EIVYKSP (SEQ ID NO:58) or EIVYKS (SEQ ID NO:277).

### BRIEF DESCRIPTION OF THE DRAWINGS

FIGS. 1A and 1B depict an alignment of heavy chain variable regions of the mouse 9F5 antibody (SEQ ID NO:7) and humanized versions of the 9F5 antibody (hu9F5VHv1, hu9F5VHv2, hu9F5VHv3, hu9F5VHv4, hu9F5VHv5, hu9F5VHv6, hu9F5VHv7, and hu9F5VHv8) with human germline heavy chain variable region sequence IGHV1-69-2*01 (SEQ ID NO:33) with human acceptor heavy chain variable region sequence AAN16432-VH_huFrwk (AAN16432_H; SEQ ID NO:31), and with human acceptor heavy chain variable region sequence 2RCS-VH_huFrwk (2RCS_H; SEQ ID NO:32). hu9F5VHv1 is SEQ ID NO:15, hu9F5VHv2 is SEQ ID NO:16, hu9F5VHv3 is SEQ ID NO:17, hu9F5VHv4 is SEQ ID NO:18, hu9F5VHv5 is SEQ ID NO:19, hu9F5VHv6 is SEQ ID NO:20, and hu9F5VHv7 is SEQ ID NO:21, and hu9F5VHv8 is SEQ ID NO:22. The CDRs of mouse 9F5 VH, as defined by Kabat/Chothia Composite, are in boldface.

FIGS. 2A and 2B depict an alignment of light chain variable regions of the mouse 9F5 antibody (SEQ ID NO:11) and humanized versions of the 9F5 antibody (hu9F5VLv1, hu9F5VLv2, hu9F5VLv3, hu9F5VLv4, hu9F5VLv5, hu9F5VLv6, and hu9F5VLv7) with human germline light chain variable region sequence IGKV2-28*01 &_IGKJ2*01 (IGKV2-28*01_IGKJ2*01; SEQ ID NO:37) with human acceptor CAB51297-VL_huFrwk (CAB51297_L; SEQ ID NO:35), and with human acceptor 1911357B-VL_huFRwk (1911357B_L; SEQ ID NO:36). hu9F5VLv1 is SEQ ID NO:23, hu9F5VLv2 is SEQ ID NO:24, hu9F5VLv3 is SEQ ID NO:25, hu9F5VLv4 is SEQ ID NO:26, hu9F5VLv5 is SEQ ID NO:27, hu9F5VLv6 is SEQ ID NO:28, and hu9F5VLv7 is SEQ ID NO:29. The CDRs of mouse 9F5 VL, as defined by Kabat, are in boldface.

FIG. 3 depicts results of an assay showing that mouse 9F5 antibody blocks neuronal internalization of tau.

FIGS. 4A and 4B depict an alignment of heavy chain variable regions of the mouse 9F5 antibody (SEQ ID NO:7) and humanized versions of the 9F5 antibody (hu9F5VHv1, hu9F5VHv2, hu9F5VHv3, hu9F5VHv4, hu9F5VHv5, hu9F5VHv6, hu9F5VHv7, hu9F5VHv8, hu9F5VHv9, and hu9F5VHv10) with human germline heavy chain variable region sequence IGHV1-69-2*01 (SEQ ID NO:33) with human acceptor heavy chain variable region sequence AAN16432-VH_huFrwk (AAN16432_H; SEQ ID NO:31), and with human acceptor heavy chain variable region sequence 2RCS-VH_huFrwk (2RCS H; SEQ ID NO:32). hu9F5VHv1 is SEQ ID NO:15, hu9F5VHv2 is SEQ ID NO:16, hu9F5VHv3 is SEQ ID NO:17, hu9F5VHv4 is SEQ ID NO:18, hu9F5VHv5 is SEQ ID NO:19, hu9F5VHv6 is SEQ ID NO:20, and hu9F5VHv7 is SEQ ID NO:21, hu9F5VHv8 is SEQ ID NO:22, hu9F5VHv9 is SEQ ID NO:127, and hu9F5VHv10 is SEQ ID NO:128. The CDRs of mouse 9F5 VH, as defined by Kabat/Chothia Composite, are in boldface. Residues identical to those of mouse 9F5 VH are indicated by “.”

FIGS. 5A and 5B depict an alignment of light chain variable regions of the mouse 9F5 antibody (SEQ ID NO:11) and humanized versions of the 9F5 antibody (hu9F5VLv1, hu9F5VLv2, hu9F5VLv3, hu9F5VLv4, hu9F5VLv5, hu9F5VLv6, hu9F5VLv7, hu9F5VLv8, and hu9F5VLv9) with human germline light chain variable region sequence IGKV2-28*01 &_IGKJ2*01 (IGKV2-28*01_IGKJ2*01; SEQ ID NO:37) with human acceptor CAB51297-VL_huFrwk (CAB51297_L; SEQ ID NO:35), and with human acceptor 1911357B-VL_huFRwk (1911357B_L; SEQ ID NO:36). hu9F5VLv1 is SEQ ID NO:23, hu9F5VLv2 is SEQ ID NO:24, hu9F5VLv3 is SEQ ID NO:25, hu9F5VLv4 is SEQ ID NO:26, hu9F5VLv5 is SEQ ID NO:27, hu9F5VLv6 is SEQ ID NO:28, hu9F5VLv7 is SEQ ID NO:2, hu9F5VLv8 is SEQ ID NO:130 and hu9F5VLv9 is SEQ ID NO:131. The CDRs of mouse 9F5 VL, as defined by Kabat, are in boldface. Residues identical to those of mouse 9F5 VL are indicated by “.”

FIGS. 6A, 6B, and 6C depict an alignment of the light chain variable region of hu9F5VLv8 with light chain variable regions of humanized versions of the 9F5 antibody: hu9F5VLv8_DIM1 (SEQ ID NO:132), hu9F5VLv8_DIM2 (SEQ ID NO:133), hu9F5VLv8_DIM3 (SEQ ID NO:134), hu9F5VLv8_DIM4 (SEQ ID NO:135), hu9F5VLv8_DIM5 (SEQ ID NO:136), hu9F5VLv8_DIM6 (SEQ ID NO:137), hu9F5VLv8_DIM7 (SEQ ID NO:138), hu9F5VLv8_DIM8 (SEQ ID NO:139), hu9F5VLv8_DIM9 (SEQ ID NO:140), hu9F5VLv8_DIM10 (SEQ ID NO:141), hu9F5VLv8_DIM11 (SEQ ID NO:142), hu9F5VLv8_DIM12 (SEQ ID NO:143), hu9F5VLv8_DIM13 (SEQ ID NO:144), hu9F5VLv8_DIM14 (SEQ ID NO:145), hu9F5VLv8_DIM15 (SEQ ID NO:146), hu9F5VLv8_DIM16 (SEQ ID NO:147), hu9F5VLv8_DIM17 (SEQ ID NO:148), hu9F5VLv8_DIM18 (SEQ ID NO:149), hu9F5VLv8_DIM19 (SEQ ID NO:150), hu9F5VLv8_DIM20 (SEQ ID NO:151), hu9F5VLv8_DIM21 (SEQ ID NO:152), hu9F5VLv8_DIM22 (SEQ ID NO:153), hu9F5VLv8_DIM23 (SEQ ID NO:154), hu9F5VLv8_DIM24 (SEQ ID NO:155), hu9F5VLv8_DIM25 (SEQ ID NO:156), hu9F5VLv8_DIM26 (SEQ ID NO:157), hu9F5VLv9_DIM1 (SEQ ID NO:162), hu9F5VLv9_DIM2 (SEQ ID NO:163), hu9F5VLv9_DIM4 (SEQ ID NO:164), hu9F5VLv9_DIM5 (SEQ ID NO:165), hu9F5VLv9_DIM8 (SEQ ID NO:166), hu9F5VLv9_DIM 10 (SEQ ID NO:167), hu9F5VLv9_DIM11 (SEQ ID NO:168), hu9F5VLv9_DIM13 (SEQ ID NO:169), hu9F5VLv9_DIM19 (SEQ ID NO:170), hu9F5VLv9_DIM20 (SEQ ID NO:171), hu9F5VLv8_DIM27 (SEQ ID NO:158), hu9F5VLv8_DIM28 (SEQ ID NO:159), hu9F5VLv8_DIM29 (SEQ ID NO:160), hu9F5VLv8_DIM30 (SEQ ID NO:161). The CDRs of hu9F5VLv8, as defined by Kabat, are in boldface.

FIG. 7 depicts an alignment of heavy chain variable regions of the mouse 10C12 antibody (SEQ ID NO:7, labeled m10C12 VH in FIG. 7) and humanized versions of the 10C12 antibody (hu10C12VHv1 and hu10C12VHv2) with human germline heavy chain variable region sequence IGHV1-69-2*01 (SEQ ID NO:33) and with human acceptor heavy chain variable region sequence CAC20421 VH (SEQ ID NO:218). hu10C12VHv1 is SEQ ID NO:214 and hu10C12VHv2 is SEQ ID NO:215. The CDRs of mouse 10C12 VH, as defined by Kabat/Chothia Composite, are in boldface.

FIG. 8 depicts an alignment of light chain variable regions of the mouse 10C12 (SEQ ID NO:11) and humanized versions of the 10C12 antibody (hu10C12VLv1 and hu10C12VLv2) with human germline light chain variable region sequence IGKV2-28*01 &_IGKJ2*01 (SEQ ID NO:37) and with human acceptor CAB51297-VL_huFrwk (SEQ ID NO:35). hu10C12VLv1 is SEQ ID NO:216 and hu10C12VLv2 is SEQ ID NO:217. The CDRs of mouse 10C12 VL, as defined by Kabat, are in boldface.

FIG. 9 depicts an alignment of heavy chain variable regions of the mouse 12C4 antibody (SEQ ID NO:219) and humanized versions of the 12C4 antibody (hu12C4VHv1 and hu12C4VHv2) with human germline heavy chain variable region sequence IGHV1-69-2*01 (SEQ ID NO:33) and with human acceptor heavy chain variable region sequence CAC20421 VH (SEQ ID NO:218). hu12C4VHv1 is SEQ ID NO:221 and hu12C4VHv2 is SEQ ID NO:222. The CDRs of mouse 12C4 VH, as defined by Kabat/Chothia Composite, are in boldface.

FIG. 10 depicts an alignment of light chain variable regions of the mouse 12C4 antibody (SEQ ID NO:11) and humanized versions of the 12C4 antibody (hu12C4VLv1 and huvVLv2) with human germline light chain variable region sequence IGKV2-28*01 &_IGKJ2*01 (SEQ ID NO:37) and with human acceptor CAB51297 (SEQ ID NO:35). hu12C4VLv1 is SEQ ID NO:223 and hu12C4VLv2 is SEQ ID NO:224. The CDRs of mouse 12C4 VL, as defined by Kabat, are in boldface.

FIG. 11 depicts an alignment of heavy chain variable regions of the mouse 17C12 antibody (SEQ ID NO:225) and humanized versions of the 17C12 antibody (hu17C12VHv1and hu17C12VHv2) with human germline heavy chain variable region sequence IGHV1-69-2*01 (SEQ ID NO:33) and with human acceptor heavy chain variable region sequence CAC20421 VH (SEQ ID NO:218). hu17C12VHv1 is SEQ ID NO:232 and hu17C12VHv2 is SEQ ID NO:233. The CDRs of mouse 17C12 VH, as defined by Kabat/Chothia Composite, are in boldface.

FIG. 12 depicts an alignment of light chain variable regions of the mouse 17C12 antibody (SEQ ID NO:228) and humanized versions of the 17C12 antibody (hu17C12VLv1 and hu17C12VLv2) with human germline light chain variable region sequence IGKV2-29*02 & IGKJ4*01 (SEQ ID NO:239) and with human acceptor QD016713 VL (SEQ ID NO:238). hu17C12VLv1 is SEQ ID NO:234 and hu17C12VLv2 is SEQ ID NO:235. The CDRs of mouse 17C12 VL, as defined by Kabat, are in boldface.

FIG. 13 depicts an alignment of heavy chain variable regions of the mouse 14H3 antibody (SEQ ID NO:240) and humanized versions of the 14H3 antibody (hu14H3VHv1 and hu14H3VHv2) with human germline heavy chain variable region sequence IGHV2-70*04 & IGHJ4*01 (SEQ ID NO:254) and with human acceptor heavy chain variable region sequence QDJ57937VH hFrwk (SEQ ID NO:253). hu14H3VHv1 is SEQ ID NO:248 and hu14H3VHv2 is SEQ ID NO:249. The CDRs of mouse 14H3 VH, as defined by Kabat/Chothia Composite, are in boldface.

FIG. 14 depicts an alignment of light chain variable regions of the mouse 14H3 antibody (SEQ ID NO:244) and humanized versions of the 14H3 antibody (hu14H3VLv1 and hu14H3VLv2) with human germline light chain variable region sequence IGKV2-28*01 &_IGKJ2*01 (IGKV2-28*01_IGKJ2*01; SEQ ID NO:37) and with human acceptor ABC66914VL_hFwrk (SEQ ID NO:256). hu14H3VLv1 is SEQ ID NO:250 and hu14H3VLv2 is SEQ ID NO:251. The CDRs of mouse 14H3 VL, as defined by Kabat, are in boldface.

FIG. 15 depicts results of an assay showing that mouse 10C12, mouse 12C4, mouse 2D11, mouse 17C12, mouse 14H3, and mouse 9F5 antibodies block neuronal internalization of tau.

FIG. 16 depicts results of an assay showing that mouse 10C12, mouse 12C4, mouse 2D11, and mouse 9F5 antibodies prevent tau toxicity in primary neurons (neuronal viability).

FIG. 17 depicts results of an assay showing that mouse 10C12, mouse 12C4, mouse 2D11, and mouse 9F5 antibodies prevent tau toxicity in primary neurons (LDH release).

FIG. 18 depicts results of a Western blot assay showing that mouse 10C12, mouse 12C4, mouse 2D11, mouse 17C12, mouse 14H3, and mouse 9F5 antibodies detect tau in samples from brains of Alzheimer's disease patients.

FIG. 19 depicts results of a immunoprecipitation assay with mouse 10C12, mouse 12C4, mouse 2D11, mouse 17C12, mouse 14H3, and mouse 9F5 antibodies and a sample from a brain of Alzheimer's disease patient.

FIG. 20 depicts results of an assay to measure ability of 9F5 humanized variants to withstand aggregation induced by agitation stress.

FIG. 21 depicts results of an assay to measure ability of 9F5 humanized variants to withstand low pH exposure.

FIG. 22 depicts results of an assay to measure ability of 9F5 humanized variants to aggregate under simulated high-concentration conditions.

FIGS. 23A-F depict results of immunohistochemistry assays using control, mouse 2D11, mouse 9F5, mouse 12C4, mouse 14H3, and mouse 17C12.

FIG. 24 depicts an alignment of heavy chain variable regions of the mouse 9F5 antibody (SEQ ID NO:7), mouse 10C12antibody (SEQ ID NO:7), mouse 2D11 antibody (SEQ ID NO:7), mouse 12C4 antibody (SEQ ID NO:219), mouse 14H3 antibody (SEQ ID NO:240), and mouse 17C12 antibody (SEQ ID NO:225). The CDRs of mouse 9F5 VH, as defined by Kabat/Chothia Composite, are in boldface.

FIG. 25 depicts an alignment of light chain variable regions of the mouse 9F5 antibody (SEQ ID NO:11), mouse 10C12antibody (SEQ ID NO:11), mouse 2D11 antibody (SEQ ID NO:11), mouse 12C4 antibody (SEQ ID NO:11), mouse 14H3 antibody (SEQ ID NO:244), and mouse 17C12 antibody (SEQ ID NO:228). The CDRs of mouse 9F5 VL, as defined by Kabat, are in boldface.

### BRIEF DESCRIPTION OF THE SEQUENCES

SEQ ID NO:1 sets forth the amino acid sequence of an isoform of human tau (Swiss-Prot P10636-8).

SEQ ID NO:2 sets forth the amino acid sequence of an isoform of human tau (Swiss-Prot P10636-7).

SEQ ID NO:3 sets forth the amino acid sequence of an isoform of human tau (Swiss-Prot P10636-6), (4R0N human tau).

SEQ ID NO:4 sets forth the amino acid sequence of an isoform of human tau (Swiss-Prot P10636-5)

SEQ ID NO:5 sets forth the amino acid sequence of an isoform of human tau (Swiss-Prot P10636-4).

SEQ ID NO:6 sets forth the amino acid sequence of an isoform of human tau (Swiss-Prot P10636-2).

SEQ ID NO:7 sets forth the amino acid sequence of the heavy chain variable region of the mouse 9F5 antibody.

SEQ ID NO:8 sets forth the amino acid sequence of Kabat/Chothia composite CDR-H1 of the mouse 9F5 antibody.

SEQ ID NO:9 sets forth the amino acid sequence of Kabat CDR-H2 of the mouse 9F5 antibody.

SEQ ID NO:10 sets forth the amino acid sequence of Kabat CDR-H3 of the mouse 9F5 antibody.

SEQ ID NO:11 sets forth the amino acid sequence of the light chain variable region of the mouse 9F5 antibody.

SEQ ID NO:12 sets forth the amino acid sequence of Kabat CDR-L1 of the mouse 9F5 antibody.

SEQ ID NO:13 sets forth the amino acid sequence of Kabat CDR-L2 of the mouse 9F5 antibody.

SEQ ID NO:14 sets forth the amino acid sequence of Kabat CDR-L3 of the mouse 9F5 antibody.

SEQ ID NO:15 sets forth the amino acid sequence of humanized heavy chain variable region hu9F5VHv1.

SEQ ID NO:16 sets forth the amino acid sequence of humanized heavy chain variable region hu9F5VHv2.

SEQ ID NO:17 sets forth the amino acid sequence of humanized heavy chain variable region hu9F5VHv3.

SEQ ID NO:18 sets forth the amino acid sequence of humanized heavy chain variable region hu9F5VHv4.

SEQ ID NO:19 sets forth the amino acid sequence of humanized heavy chain variable region hu9F5VHv5:

SEQ ID NO:20 sets forth the amino acid sequence of humanized heavy chain variable region hu9F5VHv6.

SEQ ID NO:21 sets forth the amino acid sequence of humanized heavy chain variable region hu9F5VHv7.

SEQ ID NO:22 sets forth the amino acid sequence of humanized heavy chain variable region hu9F5VHv8.

SEQ ID NO:23 sets forth the amino acid sequence of the humanized light chain variable region hu9F5VLv1.

SEQ ID NO:24 sets forth the amino acid sequence of humanized light chain variable region hu9F5VLv2.

SEQ ID NO:25 sets forth the amino acid sequence of humanized light chain variable region hu9F5VLv3.

SEQ ID NO:26 sets forth the amino acid sequence of humanized light chain variable region hu9F5VLv4.

SEQ ID NO:27 sets forth the amino acid sequence of humanized light chain variable region of the humanized 9F5 antibody hu9F5VLv5.

SEQ ID NO:28 sets forth the amino acid sequence of humanized light chain variable region hu9F5VLv6.

SEQ ID NO:29 sets forth the amino acid sequence of humanized light chain variable region hu9F5VLv7.

SEQ ID NO:30 sets forth the amino acid sequence of the heavy chain variable region structural model PDB. #5OBF-VH_mSt.

SEQ ID NO:31 sets forth the amino acid sequence of the heavy chain variable region acceptor GenBank Acc. #AAN16432-VH_huFrwk.

SEQ ID NO:32 sets forth the amino acid sequence of the heavy chain variable region acceptor PDB #2RCS-VH_huFrwk.

SEQ ID NO:33 sets forth the amino acid sequence of the heavy chain variable region germline sequence IMGT# IGHV1-69-2*01.

SEQ ID NO:34 sets forth the amino acid sequence of the light chain variable region structural model PDB #5OBF-VL_mSt.

SEQ ID NO:35 sets forth the amino acid sequence of the light chain variable region acceptor GenBank Acc. #CAB51297-VL_huFrwk.

SEQ ID NO:36 sets forth the amino acid sequence of the light chain variable region acceptor GenBank Acc. #1911357B-VL_huFrwk.

SEQ ID NO:37 sets forth the amino acid sequence of the light chain variable region germline sequence IMGT# IGKV2-28*01 & IGKJ2*01.

SEQ ID NO:38 sets forth a nucleic acid sequence encoding the heavy chain variable region of the mouse 9F5 antibody.

SEQ ID NO:39 sets forth a nucleic acid sequence encoding the light chain variable region of the mouse 9F5 antibody.

SEQ ID NO:40 sets forth the amino acid sequence of Kabat CDR-H1 of the mouse 9F5 antibody.

SEQ ID NO:41 sets forth the amino acid sequence of Chothia CDR-H1 of the mouse 9F5 antibody.

SEQ ID NO:42 sets forth the amino acid sequence of Chothia CDR-H2 of the mouse 9F5 antibody.

SEQ ID NO:43 sets forth the amino acid sequence of AbM CDR-H2 of the mouse 9F5 antibody.

SEQ ID NO:44 sets forth the amino acid sequence of Contact CDR-H1 of the mouse 9F5 antibody.

SEQ ID NO:45 sets forth the amino acid sequence of Contact CDR-H2 of the mouse 9F5 antibody.

SEQ ID NO:46 sets forth the amino acid sequence of Contact CDR-H3 of the mouse 9F5 antibody.

SEQ ID NO:47 sets forth the amino acid sequence of Contact CDR-L1 of the mouse 9F5 antibody.

SEQ ID NO:48 sets forth the amino acid sequence of Contact CDR-L2 of the mouse 9F5 antibody.

SEQ ID NO:49 sets forth the amino acid sequence of Contact CDR-L3 of the mouse 9F5 antibody.

SEQ ID NO:50 sets forth the amino acid sequence of an alternate Kabat-Chothia Composite CDR-H1 of a humanized 9F5 antibody (present in hu9F5VHv4, hu9F5VHv5, and hu9F5VHv6).

SEQ ID NO:51 sets forth the amino acid sequence of an alternate Kabat CDR-H2 of a humanized 9F5 antibody (present in hu9F5VHv5, hu9F5VHv6, and hu9F5VHv7).

SEQ ID NO:52 sets forth the amino acid sequence of an alternate Kabat CDR-H2 of a humanized 9F5 antibody (present in hu9F5VHv8).

SEQ ID NO:53 sets forth the amino acid sequence of an alternate Kabat CDR-L1 of a humanized 9F5 antibody (present in hu9F5VLv5 and hu9F5VLv6).

SEQ ID NO:54 sets forth the amino acid sequence of an alternate Kabat CDR-L1 of a humanized 9F5 antibody (present in hu9F5VLv7).

SEQ ID NO:55 sets forth the amino acid sequence of an alternate Kabat CDR-L2 of a humanized 9F5 antibody (present in hu9F5VLv4, hu9F5VLv5, hu9F5VLv6, hu9F5VLv7, hu9F5VLv8_DIM2, hu9F5VLv8_DIM4, hu9F5VLv8_DIM5, hu9F5VLv8_DIM6, hu9F5VLv8_DIM11, hu9F5VLv8_DIM12, hu9F5VLv8_DIM13, hu9F5VLv8_DIM18, hu9F5VLv8_DIM27, hu9F5VLv8_DIM28, hu9F5VLv9_DIM2, hu9F5VLv9_DIM4, hu9F5VLv9_DIM5, hu9F5VLv9_DIM11, and hu9F5VLv9_DIM13).

SEQ ID NO:56 sets forth the amino acid sequence of an epitope of antibody 9F5.

SEQ ID NO:57 sets forth the amino acid sequence of a consensus motif of a peptide bound by antibody 9F5.

SEQ ID NO:58 sets forth the amino acid sequence of a consensus motif of a peptide bound by antibody 9F5.

SEQ ID NO:59 sets forth the amino acid sequence of a linker.

SEQ ID NO:60 sets forth the amino acid sequence of an HA control peptide.

SEQ ID NO:61 sets forth the amino acid sequence of a variant of hu9F5VLv2 light chain variable region (also known as hu9F5VLv2_M51E).

SEQ ID NO:62 sets forth the amino acid sequence of a variant of hu9F5VLv2 light chain variable region (also known as hu9F5VLv2_M51D).

SEQ ID NO:63 sets forth the amino acid sequence of a variant of hu9F5VLv2 light chain variable region (also known as hu9F5VLv2_L27cD).

SEQ ID NO:64 sets forth the amino acid sequence of a variant of hu9F5VLv2 light chain variable region (also known as hu9F5VLv2_L27cG).

SEQ ID NO:65 sets forth the amino acid sequence of a variant of hu9F5VLv2 light chain variable region (also known as hu9F5VLv2_L27cS).

SEQ ID NO:66 sets forth the amino acid sequence of a variant of hu9F5VLv2 light chain variable region (also known as hu9F5VLv2_L27cE).

SEQ ID NO:67 sets forth the amino acid sequence of a variant of hu9F5VLv2 light chain variable region (also known as hu9F5VLv2_I30E).

SEQ ID NO:68 sets forth the amino acid sequence of a variant of hu9F5VLv2 light chain variable region (also known as hu9F5VLv2_I30K).

SEQ ID NO:69 sets forth the amino acid sequence of a variant of hu9F5VLv2 light chain variable region (also known as hu9F5VLv2_L27cT).

SEQ ID NO:70 sets forth the amino acid sequence of a variant of hu9F5VLv2 light chain variable region (also known as hu9F5VLv2_L27cN).

SEQ ID NO:71 sets forth the amino acid sequence of a variant of hu9F5VLv2 light chain variable region (also known as hu9F5VLv2_L27bD).

SEQ ID NO:72 sets forth the amino acid sequence of a variant of hu9F5VLv2 light chain variable region (also known as hu9F5VLv2_I30G).

SEQ ID NO:73 sets forth the amino acid sequence of a variant of hu9F5VLv2 light chain variable region (also known as hu9F5VLv2_L33N).

SEQ ID NO:74 sets forth the amino acid sequence of a variant of hu9F5VLv2 light chain variable region (also known as hu9F5VLv2_L27cA).

SEQ ID NO:75 sets forth the amino acid sequence of a variant of hu9F5VLv2 light chain variable region (also known as hu9F5VLv2_L33T).

SEQ ID NO:76 sets forth the amino acid sequence of a variant of hu9F5VLv2 light chain variable region (also known as hu9F5VLv2_L33S).

SEQ ID NO:77 sets forth the amino acid sequence of a variant of hu9F5VLv2 light chain variable region (also known as hu9F5VLv2_L33R).

SEQ ID NO:78 sets forth the amino acid sequence of a variant of hu9F5VLv2 light chain variable region (also known as hu9F5VLv2_I30Q).

SEQ ID NO:79 sets forth the amino acid sequence of a variant of hu9F5VLv2 light chain variable region (also known as hu9F5VLv2_L27bT).

SEQ ID NO:80 sets forth the amino acid sequence of a variant of hu9F5VLv2 light chain variable region (also known as hu9F5VLv2_T31G).

SEQ ID NO:81 sets forth the amino acid sequence of a variant of hu9F5VLv2 light chain variable region (also known as hu9F5VLv2_L27bQ).

SEQ ID NO:82 sets forth the amino acid sequence of a variant of hu9F5VLv2 light chain variable region (also known as hu9F5VLv2_L33G).

SEQ ID NO:83 sets forth the amino acid sequence of a variant of hu9F5VLv2 light chain variable region (also known as hu9F5VLv2_L27cP).

SEQ ID NO:84 sets forth the amino acid sequence of a variant of hu9F5VLv2 light chain variable region (also known as hu9F5VLv2_V78R).

SEQ ID NO:85 sets forth the amino acid sequence of a variant of hu9F5VLv2 light chain variable region (also known as hu9F5VLv2_I75D).

SEQ ID NO:86 sets forth the amino acid sequence of a variant of hu9F5VLv2 light chain variable region (also known as hu9F5VLv2_V78D).

SEQ ID NO:87 sets forth the amino acid sequence of a variant of hu9F5VLv2 light chain variable region (also known as hu9F5VLv2_V78E).

SEQ ID NO:88 sets forth the amino acid sequence of a variant of hu9F5VLv2 light chain variable region (also known as hu9F5VLv2_V78P).

SEQ ID NO:89 sets forth the amino acid sequence of a variant of hu9F5VLv2 light chain variable region (also known as hu9F5VLv2_V78K).

SEQ ID NO:90 sets forth the amino acid sequence of a variant of hu9F5VLv2 light chain variable region (also known as hu9F5VLv2_R77D).

SEQ ID NO:91 sets forth the amino acid sequence of a variant of hu9F5VLv2 light chain variable region (also known as hu9F5VLv2_V78G).

SEQ ID NO:92 sets forth the amino acid sequence of a variant of hu9F5VLv2 light chain variable region (also known as hu9F5VLv2_S76P).

SEQ ID NO:93 sets forth the amino acid sequence of a variant of hu9F5VLv2 light chain variable region (also known as hu9F5VLv2_I75P).

SEQ ID NO:94 sets forth the amino acid sequence of a variant of hu9F5VLv2 light chain variable region (also known as hu9F5VLv2_I75Q).

SEQ ID NO:95 sets forth the amino acid sequence of a variant of hu9F5VLv2 light chain variable region (also known as hu9F5VLv2_I75G).

SEQ ID NO:96 sets forth the amino acid sequence of a variant of hu9F5VLv2 light chain variable region (also known as hu9F5VLv2_L73P).

SEQ ID NO:97 sets forth the amino acid sequence of a variant of hu9F5VLv2 light chain variable region (also known as hu9F5VLv2_L73G).

SEQ ID NO:98 sets forth the amino acid sequence of a variant of hu9F5VLv2 light chain variable region (also known as hu9F5VLv2_V78Q).

SEQ ID NO:99 sets forth the amino acid sequence of a variant of hu9F5VLv2 light chain variable region (also known as hu9F5VLv2_S76G).

SEQ ID NO:100 sets forth the amino acid sequence of a variant of hu9F5VLv2 light chain variable region (also known as hu9F5VLv2_L92D).

SEQ ID NO:101 sets forth the amino acid sequence of a variant of hu9F5VLv2 light chain variable region (also known as hu9F5VLv2_Y86T).

SEQ ID NO:102 sets forth the amino acid sequence of a variant of hu9F5VLv2 light chain variable region (also known as hu9F5VLv2_L92E).

SEQ ID NO:103 sets forth the amino acid sequence of a variant of hu9F5VLv2 light chain variable region (also known as hu9F5VLv2_L92G).

SEQ ID NO:104 sets forth the amino acid sequence of a variant of hu9F5VLv2 light chain variable region (also known as hu9F5VLv2_L92Q).

SEQ ID NO:105 sets forth the amino acid sequence of a variant of hu9F5VLv2 light chain variable region (also known as hu9F5VLv2_L93G).

SEQ ID NO:106 sets forth the amino acid sequence of a variant of hu9F5VLv2 light chain variable region (also known as hu9F5VLv2_V85G).

SEQ ID NO:107 sets forth the amino acid sequence of a variant of hu9F5VLv2 light chain variable region (also known as hu9F5VLv2_L92T).

SEQ ID NO:108 sets forth the amino acid sequence of a variant of hu9F5VLv2 light chain variable region (also known as hu9F5VLv2_A89G).

SEQ ID NO:109 sets forth the amino acid sequence of a variant of hu9F5VHv4 heavy chain variable region (also known as hu9F5VHv4_L80P).

SEQ ID NO:110 sets forth the amino acid sequence of a variant of hu9F5VHv4 heavy chain variable region (also known as hu9F5VHv4_L80D).

SEQ ID NO:111 sets forth the amino acid sequence of a variant of hu9F5VHv4 heavy chain variable region (also known as hu9F5VHv4_L82cG).

SEQ ID NO:112 sets forth the amino acid sequence of a variant of hu9F5VHv4 heavy chain variable region (also known as hu9F5VHv4_L82cD).

SEQ ID NO:113 sets forth the amino acid sequence of a variant of hu9F5VHv4 heavy chain variable region (also known as hu9F5VHv4_L82P).

SEQ ID NO:114 sets forth the amino acid sequence of a variant of hu9F5VHv4 heavy chain variable region (also known as hu9F5VHv4_L80G).

SEQ ID NO:115 sets forth the amino acid sequence of a variant of hu9F5VHv4 heavy chain variable region (also known as hu9F5VHv4_L82K).

SEQ ID NO:116 sets forth the amino acid sequence of a variant of hu9F5VHv4 heavy chain variable region (also known as hu9F5VHv4_L82R).

SEQ ID NO:117 sets forth the amino acid sequence of a variant of hu9F5VHv4 heavy chain variable region (also known as hu9F5VHv4_L82E).

SEQ ID NO:118 sets forth the amino acid sequence of a variant of hu9F5VHv4 heavy chain variable region (also known as hu9F5VHv4_L82N).

SEQ ID NO:119 sets forth the amino acid sequence of a variant of hu9F5VHv4 heavy chain variable region (also known as hu9F5VHv4_Y79D).

SEQ ID NO:120 sets forth the amino acid sequence of a variant of hu9F5VHv4 heavy chain variable region (also known as hu9F5VHv4_Y79N).

SEQ ID NO:121 sets forth the amino acid sequence of a variant of hu9F5VHv4 heavy chain variable region (also known as hu9F5VHv4_Y79G).

SEQ ID NO:122 sets forth the amino acid sequence of a variant of hu9F5VHv5 heavy chain variable region (also known as hu9F5VHv5_M80E).

SEQ ID NO:123 sets forth the amino acid sequence of a variant of hu9F5VHv5 heavy chain variable region (also known as hu9F5VHv5_M80G).

SEQ ID NO:124 sets forth the amino acid sequence of a variant of hu9F5VHv4 heavy chain variable region (also known as hu9F5VHv4_L82cS).

SEQ ID NO:125 sets forth the amino acid sequence of a variant of hu9F5VHv4 heavy chain variable region (also known as hu9F5VHv4_Y79Q).

SEQ ID NO:126 sets forth the amino acid sequence of a variant of hu9F5VHv4 heavy chain variable region (also known as hu9F5VHv4_S82aG).

SEQ ID NO:127 sets forth the amino acid sequence of a Heavy chain variable region hu9F5VHv9.

SEQ ID NO:128 sets forth the amino acid sequence of a Heavy chain variable region hu9F5VHv10 (also known as hu9F5VHv9_Q38K_G42E).

SEQ ID NO:129 sets forth the amino acid sequence of a Heavy chain variable region hu9F5VHv10_L82cG.

SEQ ID NO: 130 sets forth the amino acid sequence of a Light chain variable region hu9F5VLv8.

SEQ ID NO:131 sets forth the amino acid sequence of a Light chain variable region hu9F5VLv9 (also known as hu8F5VLv8_N60D),

SEQ ID NO:132 sets forth the amino acid sequence of a variant of hu9F5VLv8 light chain variable region (hu9F5VLv8_V3Q, L27cS, L37Q, M51G, L54G, L92I, also known as hu9F5VLv8_DIM1).

SEQ ID NO:133 sets forth the amino acid sequence of a variant of hu9F5VLv8 light chain variable region (hu9F5VLv8_V3Q, L27cS, L37Q, M51G, L54R, L92I, also known as hu9F5VLv8_DIM2).

SEQ ID NO:134 sets forth the amino acid sequence of a variant of hu9F5VLv8 light chain variable region (hu9F5VLv8_V3Q, L27cS, L37Q, M51G, L54T, L92I, also known as hu9F5VLv8_DIM5).

SEQ ID NO:135 sets forth the amino acid sequence of a variant of hu9F5VLv8 light chain variable region (hu9F5VLv8_V3Q, L27cS, L37Q, M51G, L54R, L92G, also known as hu9F5VLv8_DIM4).

SEQ ID NO:136 sets forth the amino acid sequence of a variant of hu9F5VLv8 light chain variable region (hu9F5VLv8_V3Q, L27cG, L37Q, M51G, L54R, L92I, also known as hu9F5VLv8_DIM5).

SEQ ID NO:137 sets forth the amino acid sequence of a variant of hu9F5VLv8 light chain variable region (hu9F5VLv8_V3Q, L27cD, L37Q, M51G, L54R, L92I, also known as hu9F5VLv8_DIM6)

SEQ ID NO: 138 sets forth the amino acid sequence of a variant of hu9F5VLv8 light chain variable region (hu9F5VLv8_V3Q, L27cD, L37Q, M51K, L54R, L92I, also known as hu9F5VLv8_DIM 7).

SEQ ID NO:139 sets forth the amino acid sequence of a variant of hu9F5VLv8 light chain variable region (hu9F5VLv8_V3Q, L27cG, L37Q, M51K, L54R, L92I, also known as hu9F5VLv8_DIM8)

SEQ ID NO:140 sets forth the amino acid sequence of a Variant of hu9F5VLv8 light chain variable region (hu9F5VLv8_V3Q, L27cG, L37Q, M51K, L54G, L92I, also known as hu9F5VLv8_DIM9).

SEQ ID NO:141 sets forth the amino acid sequence of a Variant of hu9F5VLv8 light chain variable region (hu9F5VLv8_V3Q, L27cS, L37Q, M51K, L54G, L92I, also known as hu9F5VLv8_DIM 10).

SEQ ID NO:142 sets forth the amino acid sequence of a variant of hu9F5VLv8 light chain variable region (hu9F5VLv8_V3Q, L27cG, L37G, M51G, L54R, L92I, also known as hu9F5VLv8_DIM11)

SEQ ID NO:143 sets forth the amino acid sequence of a variant of hu9F5VLv8 light chain variable region (hu9F5VLv8_V3Q, L27cG, L37G, M51G, L54R, L92G, also known as hu9F5VLv8_DIM 2)).

SEQ ID NO:144 sets forth the amino acid sequence of a variant of hu9F5VLv8 light chain variable region (hu9F5VLv8_V3Q, L27cG, L37G, M51G, L54R, also known as hu9F5 VLv8_DIM13).

SEQ ID NO:145 sets forth the amino acid sequence of a variant of hu9F5VLv8 light chain variable region (hu9F5VLv8_V3Q, L27cG, L37G, M51G, L54T, L92I, also known as hu9F5VLv8_DIM14)

SEQ ID NO:146 sets forth the amino acid sequence of a variant of hu9F5VLv8 light chain variable region (hu9F5VLv8_V3Q, L27cG, L37G, M51G, L54T, L92G, also known as hu9F5 VLv8_DIM15).

SEQ ID NO:147 sets forth the amino acid sequence of a variant of hu9F5VLv8 light chain variable region (hu9F5VLv8_V3Q, L27cG, L37G, M51G, L54T, also known as hu9F5VLv8_DIM16)

SEQ ID NO:148 sets forth the amino acid sequence of a variant of hu9F5VLv8 light chain variable region (hu9F5VLv8_V3Q, L27cS, L37G, M51G, L54T, L92I, also known as hu9F5VLv8_DIM 17).

SEQ ID NO:149 sets forth the amino acid sequence of a variant of hu9F5VLv8 light chain variable region (hu9F5VLv8_V3Q, L27cD, L37G, M51G, L54R, L92I, also known as hu9F5VLv8_DIM18).

SEQ ID NO:150 sets forth the amino acid sequence of a variant of hu9F5VLv8 light chain variable region (hu9F5VLv8_V3Q, L27cS, L37I, M51I, L54R, L92I, also known as hu9F5VLv8_DIM19)

SEQ ID NO:151 sets forth the amino acid sequence of a variant of hu9F5VLv8 light chain variable region (hu9F5VLv8_V3Q, L27cS, L37Q, M51I, L54G, L92I, also known as hu9F5VLv8_DIM20).

SEQ ID NO:152 sets forth the amino acid sequence of a variant of hu9F5VLv8 light chain variable region (hu9F5VLv8_V3Q, L27cS, L37Q, M51I, L54G, also known as hu9F5VLv8_DIM21).

SEQ ID NO:153 sets forth the amino acid sequence of a variant of hu9F5VLv8 light chain variable region (hu9F5VLv8_V3Q, L27cS, L37Q, M51E, L54R, L92I, also known as hu9F5VLv8_DIM22)

SEQ ID NO:154 Variant of hu9F5VLv8 light chain variable region (hu9F5VLv8_V3Q, L27cG, L37Q, M51E, L54G, L92I, also known as hu9F5VLv8_DIM23).

SEQ ID NO:155 sets forth the amino acid sequence of a variant of hu9F5VLv8 light chain variable region (hu9F5VLv8_V3Q, L27cG, L37I, M51E, L54R, L92I, also known as hu9F5VLv8_DIM24).

SEQ ID NO:156 sets forth the amino acid sequence of a variant of hu9F5VLv8 light chain variable region (hu9F5VLv8_V3Q, L27cG, L37I, M51E, L54R, L92G, also known as hu9F5VLv8_DIM25)

SEQ ID NO:157 sets forth the amino acid sequence of a variant of hu9F5VLv8 light chain variable region (hu9F5VLv8_V3Q, L27cI, L37I, M51E, L54R, also known as hu9F5VLv8_DIM26).

SEQ ID NO:158 sets forth the amino acid sequence of a variant of hu9F5VLv8 light chain variable region (hu9F5VLv8_V3Q, L37Q, M51G, L54R, L92I, also known as hu9F5VLv8_DIM27).

SEQ ID NO:159 sets forth the amino acid sequence of a variant of hu9F5VLv8 light chain variable region (hu9F5VLv8_V3Q, L27cS, M51G, L54R, L92I, also known as hu9F5VLv8_DIM28)

SEQ ID NO:160 sets forth the amino acid sequence of a variant of hu9F5VLv8 light chain variable region (hu9F5VLv8_V3Q, L27cS, L37Q, L54R, L92I, also known as hu9F5VLv8_DIM29).

SEQ ID NO:161 sets forth the amino acid sequence of a variant of hu9F5VLv8 light chain variable region (hu9F5VLv8_V3Q, L27cS, L37Q, M51G, L92I, also known as hu9F5VLv8_DIM30).

SEQ ID NO:162 sets forth the amino acid sequence of a variant of hu9F5VLv9 light chain variable region (hu9F5VLv9_V3Q, L27cS, L37Q, M51G, L54G, L92I, also known as hu9F5VLv9_DIM1).

SEQ ID NO:163 sets forth the amino acid sequence of a variant of hu9F5VLv9 light chain variable region (hu9F5VLv9_V3Q, L27cS, L37Q, M51G, L54R, L92I, also known as hu9F5VLv9_DIM2).

SEQ ID NO:164 sets forth the amino acid sequence of a variant of hu9F5VLv9 light chain variable region (hu9F5VLv9_V3Q, L27cS, L37Q, M51G, L54R, L92G, also known as hu9F5VLv9_DIM4)

SEQ ID NO:165 sets forth the amino acid sequence of a variant of hu9F5VLv9 light chain variable region (hu9F5VLv9_V3Q, L27cG, L37Q, M51G, L54R, L92I, also known as hu9F5VLv9_DIM5).

SEQ ID NO:166 sets forth the amino acid sequence of a variant of hu9F5VLv9 light chain variable region (hu9F5VLv9_V3Q, L27cG, L37Q, M51K, L54R, L92I, also known as hu9F5VLv9_DIM8).

SEQ ID NO:167 sets forth the amino acid sequence of a variant of hu9F5VLv9 light chain variable region (hu9F5VLv9_V3Q, L27cS, L37Q, M51K, L54G, L92I, also known as 9F5 VLv9_DIM10)

SEQ ID NO:168 sets forth the amino acid sequence of a variant of hu9F5VLv9 light chain variable region (hu9F5VLv9_V3Q, L27cG, L37G, M51G, L54R, L92I, also known as hu9F5VLv9_DIM11).

SEQ ID NO:169 sets forth the amino acid sequence of a variant of hu9F5VLv9 light chain variable region (hu9F5VLv9_V3Q, L27cG, L37G, M51G, L54R, also known as hu9F5VLv9_DIM13).

SEQ ID NO:170 sets forth the amino acid sequence of a variant of hu9F5VLv9 light chain variable region (hu9F5VLv9_V3Q, L27cS, L37I, M51I, L54R, L92I, also known as hu9F5VLv9_DIM 19)

SEQ ID NO:171 sets forth the amino acid sequence of a variant of hu9F5VLv9 light chain variable region (hu9F5VLv9_V3Q, L27cS, L37Q, M51I, L54G, L92I, also known as hu9F5VLv9_DIM20).

SEQ ID NO:172 sets forth the amino acid sequence of an alternate Kabat CDR-L1 of a humanized 9F5 antibody (present in hu9F5VLv2_L27bD).

SEQ ID NO:173 sets forth the amino acid sequence of an alternate Kabat CDR-L1 of a humanized 9F5 antibody (present in hu9F5VLv2_L27bT).

SEQ ID NO:174 sets forth the amino acid sequence of an alternate Kabat CDR-L1 of a humanized 9F5 antibody (present in hu9F5VLv2_L27bQ).

SEQ ID NO:175 sets forth the amino acid sequence of an alternate Kabat CDR-L1 of a humanized 9F5 antibody (present in hu9F5VLv2_L27cD, hu9F5VLv8_DIM6, hu9F5VLv8_DIM7, and hu9F5VLv8_DIM18).

SEQ ID NO:176 sets forth the amino acid sequence of an alternate Kabat CDR-L1 of a humanized 9F5 antibody (present in hu9F5VLv2_L27cG, hu9F5VLv8_DIM5, hu9F5VLv8_DIM8, hu9F5VLv8_DIM9, in hu9F5VLv8_DIM11, hu9F5VLv8_DIM12, hu9F5VLv8_DIM13, hu9F5VLv8_DIM14, hu9F5VLv8_DIM15, hu9F5VLv8_DIM16, hu9F5VLv8_DIM23, hu9F5VLv8_DIM24, hu9F5VLv8_DIM25, hu9F5VLv9_DIM5, hu9F5VLv9_DIM8, hu9F5VLv9_DIM11, and hu9F5VLv9_DIM13).

SEQ ID NO:177 sets forth the amino acid sequence of an alternate Kabat CDR-L1 of a humanized 9F5 antibody (present in hu9F5VLv2_L27cS, hu9F5VLv8_DIM1, hu 9F5VLv8_DIM2, hu9F5VLv8_DIM19, hu9F5VLv8_DIM20, hu9F5VLv8_DIM21, hu9F5VLv8_DIM22, hu9F5VLv8_DIM28, hu9F5VLv8_DIM29, hu9F5VLv8_DIM30, hu9F5VLv9_DIM1, hu9F5VLv9_DIM2, hu9F5VLv9_DIM4, hu9F5VLv9_DIM10, in hu9F5VLv9_DIM19, and hu9F5VLv9_DIM20).

SEQ ID NO:178 sets forth the amino acid sequence of an alternate Kabat CDR-L1 of a humanized 9F5 antibody (present in hu9F5VLv2_L27cE).

SEQ ID NO:179 sets forth the amino acid sequence of an alternate Kabat CDR-L1 of a humanized 9F5 antibody (present in hu9F5VLv2_L27cT).

SEQ ID NO:180 sets forth the amino acid sequence of an alternate Kabat CDR-L1 of a humanized 9F5 antibody (present in hu9F5VLv2_L27cN).

SEQ ID NO:181 sets forth the amino acid sequence of an alternate Kabat CDR-L1 of a humanized 9F5 antibody (present in hu9F5VLv2_L27cA).

SEQ ID NO:182 sets forth the amino acid sequence of an alternate Kabat CDR-L1 of a humanized 9F5 antibody (present in hu9F5VLv2_L27cP).

SEQ ID NO:183 sets forth the amino acid sequence of an alternate Kabat CDR-L1 of a humanized 9F5 antibody (present in hu9F5VLv8_DIM26).

SEQ ID NO:184 sets forth the amino acid sequence of an alternate Kabat CDR-L1 of a humanized 9F5 antibody (present in hu9F5VLv2_I30E).

SEQ ID NO:185 sets forth the amino acid sequence of an alternate Kabat CDR-L1 of a humanized 9F5 antibody (present in hu9F5VLv2_I30K).

SEQ ID NO:186 sets forth the amino acid sequence of an alternate Kabat CDR-L1 of a humanized 9F5 antibody (present in hu9F5VLv2_I30G).

SEQ ID NO:187 sets forth the amino acid sequence of an alternate Kabat CDR-L1 of a humanized 9F5 antibody (present in hu9F5VLv2_I30Q).

SEQ ID NO:188 sets forth the amino acid sequence of an alternate Kabat CDR-L1 of a humanized 9F5 antibody (present in hu9F5VLv2_T31G).

SEQ ID NO:189 sets forth the amino acid sequence of an alternate Kabat CDR-L1 of a humanized 9F5 antibody (present in hu9F5VLv2_L33N).

SEQ ID NO:190 sets forth the amino acid sequence of an alternate Kabat CDR-L1 of a humanized 9F5 antibody (present in hu9F5VLv2_L33T).

SEQ ID NO:191 sets forth the amino acid sequence of an alternate Kabat CDR-L1 of a humanized 9F5 antibody (present in hu9F5VLv2_L33S).

SEQ ID NO:192 sets forth the amino acid sequence of an alternate Kabat CDR-L1 of a humanized 9F5 antibody (present in hu9F5VLv2_L33R).

SEQ ID NO:193 sets forth the amino acid sequence of an alternate Kabat CDR-L1 of a humanized 9F5 antibody (present in hu9F5VLv2_L33G).

SEQ ID NO:194 sets forth the amino acid sequence of an alternate Kabat CDR-L2 of a humanized 9F5 antibody (present in hu9F5VLv2_M51E).

SEQ ID NO:195 sets forth the amino acid sequence of an alternate Kabat CDR-L2 of a humanized 9F5 antibody (present in hu9F5VLv2_M51D).

SEQ ID NO:196 sets forth the amino acid sequence of an alternate Kabat CDR-L2 of a humanized 9F5 antibody (present in hu9F5VLv8_DIM30).

SEQ ID NO:197 sets forth the amino acid sequence of an alternate Kabat CDR-L2 of a humanized 9F5 antibody (present in hu9F5VLv8_DIM29).

SEQ ID NO:198 sets forth the amino acid sequence of an alternate Kabat CDR-L2 of a humanized 9F5 antibody (present in hu9F5VLv8_DIM1, and hu9F5VLv9_DIM1).

SEQ ID NO:199 sets forth the amino acid sequence of an alternate Kabat CDR-L2 of a humanized 9F5 antibody (present in hu9F5VLv8_DIM3, hu9F5VLv8_DIM14, hu9F5VLv8_DIM15, hu9F5VLv8_DIM16, and hu9F5VLv8_DIM17).

SEQ ID NO:200 sets forth the amino acid sequence of an alternate Kabat CDR-L2 of a humanized 9F5 antibody (present in hu9F5VLv8_DIM7, hu9F5VLv8_DIM5, and hu9F5VLv9_DIM8).

SEQ ID NO:201 sets forth the amino acid sequence of an alternate Kabat CDR-L2 of a humanized 9F5 antibody (present in hu9F5VLv8_DIM9, hu9F5VLv8_DIM10, and hu9F5VLv9_DIM10).

SEQ ID NO:202 sets forth the amino acid sequence of an alternate Kabat CDR-L2 of a humanized 9F5 antibody (present in hu9F5VLv8_DIM19, and hu9F5VLv9_DIM19).

SEQ ID NO:203 sets forth the amino acid sequence of an alternate Kabat CDR-L2 of a humanized 9F5 antibody (present in hu9F5VLv8_DIM20, hu9F5VLv8_DIM21, and hu9F5VLv9_DIM20).

SEQ ID NO:204 sets forth the amino acid sequence of an alternate Kabat CDR-L2 of a humanized 9F5 antibody (present in hu9F5VLv8_DIM22, hu9F5VLv8_DIM24, hu9F5VLv8_DIM25, and hu9F5VLv8_DIM26).

SEQ ID NO:205 sets forth the amino acid sequence of an alternate Kabat CDR-L2 of a humanized 9F5 antibody (present in hu9F5VLv8_DIM23).

SEQ ID NO:206 sets forth the amino acid sequence of an alternate Kabat CDR-L3 of a humanized 9F5 antibody (present in hu9F5VLv2_A89G).

SEQ ID NO:207 sets forth the amino acid sequence of an alternate Kabat CDR-L3 of a humanized 9F5 antibody (present in hu9F5VLv2_L92D).

SEQ ID NO:208 sets forth the amino acid sequence of an alternate Kabat CDR-L3 of a humanized 9F5 antibody (present in hu9F5VLv2_L92E).

SEQ ID NO:209 sets forth the amino acid sequence of an alternate Kabat CDR-L3 of a humanized 9F5 antibody (present in hu9F5VLv8_DIM4, hu9F5 VLv8_DIM12, hu9F5VLv8_DIM15, hu9F5VLv8_DIM25, and hu9F5VLv9_DIM4).

SEQ ID NO:210 sets forth the amino acid sequence of an alternate Kabat CDR-L3 of a humanized 9F5 antibody (present in hu9F5VLv2_L92Q).

SEQ ID NO:211 sets forth the amino acid sequence of an alternate Kabat CDR-L3 of a humanized 9F5 antibody (present in hu9F5VLv2_L92T).

SEQ ID NO:212 sets forth the amino acid sequence of an alternate Kabat CDR-L3 of a humanized 9F5 antibody (present in hu9F5VLv8_DIM1, hu9F5VLv8_DIM2, hu9F5VLv8_DIM3, hu9F5VLv8_DIM5, hu9F5VLv8DIM6, hu8F5VLv8_DIM7, hu9F5VLv8_DIM8, hu9F5VLv8_DIM9, hu9F5VLv8_DIM10, hu9F5VLv8_DIM11, hu9F5VLv8_DIM14, hu9F5VLv8DIM17, hu9F5VLv8_DIM18, hu9F5VLv8_DIM19, hu9F5VLv8_DIM20, hu9F5VLv8_DIM22, hu9F5VLv8_DIM23, hu9F5VLv8DIM24, hu9F5VLv8_DIM27, hu9F5VLv8_DIM28, hu9F5VLv8_DIM29, hu8F5VLv8_DIM30, hu9F5VLv9_DIM1, hu9F5VLv9_DIM2, hu9F5VLv9_DIM5, hu9F5VLv9_DIM8, hu9F5VLv9_DIM10, hu9F5VLv9_DIM11, hu9F5VLv9_DIM19, and u9F5VLv9_DIM20).

SEQ ID NO:213 sets forth the amino acid sequence of an alternate Kabat CDR-L3 of a humanized 9F5 antibody (present in hu9F5VLv2_L93G).

SEQ ID NO:214 sets forth the amino acid sequence of humanized heavy chain variable region hu10C12VHv1.

SEQ ID NO:215 sets forth the amino acid sequence of humanized heavy chain variable region hu10C12VHv2.

SEQ ID NO:216 sets forth the amino acid sequence of humanized light chain variable region hu10C12VLv1.

SEQ ID NO:217 sets forth the amino acid sequence of humanized light chain variable region hu10C12VLv2.

SEQ ID NO:218 sets forth the amino acid sequence of heavy chain variable region acceptor CAC20421-VH_huFrwk.

SEQ ID NO:219 sets forth the amino acid sequence of the heavy chain variable region of the mouse 12C4 antibody.

SEQ ID NO:220 sets forth the amino acid sequence of Kabat CDR-H2 of the mouse 12C4 antibody.

SEQ ID NO:221 sets forth the amino acid sequence of humanized heavy chain variable region hu12C4VHv1.

SEQ ID NO:222 sets forth the amino acid sequence of humanized heavy chain variable region hu12C4VHv2.

SEQ ID NO:223 sets forth the amino acid sequence of humanized light chain variable region hu12C4VLv1.

SEQ ID NO:224 sets forth the amino acid sequence of humanized light chain variable region hu12C4VLv2.

SEQ ID NO:225 sets forth the amino acid sequence of the heavy chain variable region of the mouse 17C12 antibody.

SEQ ID NO:226 sets forth the amino acid sequence of Kabat-Chothia composite CDR H1 of the mouse 17C12 antibody.

SEQ ID NO:227 sets forth the amino acid sequence of Kabat CDR H2 of the mouse 17C12 antibody.

SEQ ID NO:228 sets forth the amino acid sequence of the light chain variable region of the mouse 17C12 antibody.

SEQ ID NO:229 sets forth the amino acid sequence of Kabat CDR-L1 of the mouse 17C12 antibody.

SEQ ID NO:230 sets forth the amino acid sequence of Kabat CDR-L2 of the mouse 17C12 antibody.

SEQ ID NO:231 sets forth the amino acid sequence of Kabat CDR-L3 of the mouse 17C12 antibody.

SEQ ID NO:232 sets forth the amino acid sequence of humanized heavy chain variable region hu17C12VHv1.

SEQ ID NO:233 sets forth the amino acid sequence of humanized heavy chain variable region hu17C12VHv2.

SEQ ID NO:234 sets forth the amino acid sequence of humanized light chain variable region hu17C12VLv1.

SEQ ID NO:235 sets forth the amino acid sequence of humanized light chain variable region hu17C12VLv2.

SEQ ID NO:236 sets forth the amino acid sequence of the heavy chain variable region structural model 3PP3-VH_mSt.

SEQ ID NO:237 sets forth the amino acid sequence of the light chain variable region structural model 3PP3-VL_mSt.

SEQ ID NO:238 sets forth the amino acid sequence of the light chain variable region acceptor QDO16713-VL_huFrwk.

SEQ ID NO:239 sets forth the amino acid sequence of the light chain variable region germline sequence IGKV2-29*02 & IGKJ4*01.

SEQ ID NO:240 sets forth the amino acid sequence of the heavy chain variable region of the mouse 14H3 antibody.

SEQ ID NO:241 sets forth the amino acid sequence of Kabat-Chothia composite CDR H1 of the mouse 14H3 antibody.

SEQ ID NO:242 sets forth the amino acid sequence of Kabat CDR H2 of the mouse 14H3 antibody.

SEQ ID NO:243 sets forth the amino acid sequence of Kabat CDR H3 of the mouse 14H3 antibody.

SEQ ID NO:244 sets forth the amino acid sequence of the light chain variable region of the mouse 14H3 antibody.

SEQ ID NO:245 sets forth the amino acid sequence of Kabat CDR L1 of the mouse 14H3 antibody

SEQ ID NO:246 sets forth the amino acid sequence of Kabat CDR L2 of the mouse 14H3 antibody.

SEQ ID NO:247 sets forth the amino acid sequence of Kabat CDR L3 of the mouse 14H3 antibody.

SEQ ID NO:248 sets forth the amino acid sequence of humanized heavy chain variable region hu14H3VHv1.

SEQ ID NO:249 sets forth the amino acid sequence of humanized heavy chain variable region hu14H3VHv2.

SEQ ID NO:250 sets forth the amino acid sequence of humanized light chain variable region hu14H3VLv1.

SEQ ID NO:251 sets forth the amino acid sequence of humanized light chain variable region hu14H3VLv2.

SEQ ID NO:252 sets forth the amino acid sequence of the heavy chain variable region structural model 2VQ1-VH_mSt.

SEQ ID NO:253 sets forth the amino acid sequence of the heavy chain variable region acceptor QDJ57937-VH_huFrwk.

SEQ ID NO:254 sets forth the amino acid sequence of the heavy chain variable region germline sequence IGHV1-70*04 & IGHJ4*01.

SEQ ID NO:255 sets forth the amino acid sequence of the light chain variable region structural model 2VQ1-VL_mSt.

SEQ ID NO:256 sets forth the amino acid sequence of the light chain variable region acceptor ABC66914-VL_huFrwk.

SEQ ID NO:257 sets forth the amino acid sequence of AbM CDR-H2 of the mouse 12C4 antibody.

SEQ ID NO:258 sets forth the amino acid sequence of Contact CDR-H2 of the mouse 12C4 antibody.

SEQ ID NO:259 sets forth the amino acid sequence of Chothia CDR-H1 of the mouse 17C12 antibody.

SEQ ID NO:260 sets forth the amino acid sequence of AbM CDR-H2 of the mouse 17C12 antibody

SEQ ID NO:261 sets forth the amino acid sequence of Contact CDR-H2 of the mouse 17C12 antibody.

SEQ ID NO:262 sets forth the amino acid sequence of Contact CDR-L1 of the mouse 17C12 antibody.

SEQ ID NO:263 sets forth the amino acid sequence of Contact CDR-L2 of the mouse 17C12 antibody.

SEQ ID NO:264 sets forth the amino acid sequence of Contact CDR-L3 of the mouse 17C12 antibody.

SEQ ID NO:265 sets forth the amino acid sequence of Kabat CDR-H1 of the mouse 14H3 antibody.

SEQ ID NO:266 sets forth the amino acid sequence of Chothia CDR-H1 of the mouse 14H3 antibody.

SEQ ID NO:267 sets forth the amino acid sequence of Chothia CDR-H2 of the mouse 14H3 antibody.

SEQ ID NO:268 sets forth the amino acid sequence of AbM CDR-H2 of the mouse 14H3 antibody.

SEQ ID NO:269 sets forth the amino acid sequence of Contact CDR-H1 of the mouse 14H3 antibody.

SEQ ID NO:270 sets forth the amino acid sequence of Contact CDR-H2 of the mouse 14H3 antibody.

SEQ ID NO:271 sets forth the amino acid sequence of Contact CDR-H3 of the mouse 14H3 antibody.

SEQ ID NO:272 sets forth the amino acid sequence of Contact CDR-L1 of the mouse 14H3 antibody.

SEQ ID NO:273 sets forth the amino acid sequence of Contact CDR-L2 of the mouse 14H3 antibody.

SEQ ID NO:274 sets forth the amino acid sequence of Contact CDR-L3 of the mouse 14H3 antibody.

SEQ ID NO:275 sets forth the amino acid sequence of an alternate Kabat-Chothia composite CDR H1 of a humanized 14H3 antibody (present in hu14H3VHv1 and hu14H3VHv2).

SEQ ID NO:276 sets forth the amino acid sequence of a consensus motif of a peptide bound by antibody 9F5, 10C12, 2D11, 12C4, 17C12, and 14H3.

SEQ ID NO:277 sets forth the amino acid sequence of a consensus motif of a peptide bound by antibody 2D11.

### Definitions

Monoclonal antibodies or other biological entities are typically provided in isolated form. This means that an antibody or other biologically entity is typically at least 50% w/w pure of interfering proteins and other contaminants arising from its production or purification but does not exclude the possibility that the monoclonal antibody is combined with an excess of pharmaceutically acceptable carrier(s) or other vehicle intended to facilitate its use. Sometimes monoclonal antibodies are at least 60%, 70%, 80%, 90%, 95% or 99% w/w pure of interfering proteins and contaminants from production or purification. Often an isolated monoclonal antibody or other biological entity is the predominant macromolecular species remaining after its purification.

Specific binding of an antibody to its target antigen means an affinity and/or avidity of at least 10⁶, 10⁷, 10⁸, 10⁹, 10¹⁰, 10¹¹, or 10¹² M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody binds one and only one target.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. See generally, Fundamental Immunology, Paul, W., ed., 2nd ed. Raven Press, N.Y., 1989, Ch. 7 (incorporated by reference in its entirety for all purposes).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3. When the application discloses a VL sequence with R as the C-terminal residue, the R can alternatively be considered as being the N-terminal residue of the light chain constant region. Thus, the application should also be understood as disclosing the VL sequence without the C-terminal R.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modeling software; and, the contact definition of Martin et al (bioinfo.org.uk/abs) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

TABLE 1
Conventional Definitions of CDRs Using Kabat Numbering

| | | | Composite of | | |
|---|---|---|---|---|---|
| Loop | Kabat | Chothia | Chothia & Kabat | AbM | Contact |
| L1 | L24--L34 | L24--L34 | L24--L34 | L24--L34 | L30--L36 |
| L2 | L50--L56 | L50--L56 | L50--L56 | L50--L56 | L46--L55 |
| L3 | L89--L97 | L89--L97 | L89--L97 | L89--L97 | L89--L96 |
| H1 | H31--H35B | H26--H32 . . . H34* | H26--H35B* | H26--H35B | H30--H35B |
| H2 | H50--H65 | H52--H56 | H50--H65 | H50--H58 | H47--H58 |
| H3 | H95--H102 | H95--H102 | H95--H102 | H95--H102 | H93--H101 |

*CDR-H1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia CDR-H1 loop ends at H32. If only H35A is present, it ends at H33. If both H35A and H35B are present, it ends at H34.

The term “antibody” includes intact antibodies and binding fragments thereof. Typically, fragments compete with the intact antibody from which they were derived for specific binding to the target including separate heavy chains, light chains Fab, Fab′, F(ab′)2, F(ab)c, Dabs, nanobodies, and Fv. Fragments can be produced by recombinant DNA techniques, or by enzymatic or chemical separation of intact immunoglobulins. The term “antibody” also includes a bispecific antibody and/or a humanized antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)). In some bispecific antibodies, the two different heavy/light chain pairs include a humanized 9F5, 10C12, 2D11, 12C4, 17C12, or 14H3 heavy chain/light chain pair and a heavy chain/light chain pair specific for a different epitope on tau than that bound by 9F5, 10C12, 2D11, 12C4, 17C12, or 14H3.

In some bispecific antibodies, one heavy chain/light chain pair is a humanized 9F5 antibody, humanized 10C12 antibody, humanized 2D11 antibody, humanized 12C4 antibody, humanized 17C12 antibody, or humanized 14H3 antibody as further disclosed below and the other heavy chain/light chain pair is from an antibody that binds to a receptor expressed on the blood brain barrier, such as an insulin receptor, an insulin-like growth factor (IGF) receptor, a leptin receptor, or a lipoprotein receptor, or a transferrin receptor (Friden et al., Proc. Natl. Acad. Sci. USA 88:4771-4775, 1991; Friden et al., Science 259:373-377, 1993). Such a bispecific antibody can be transferred cross the blood brain barrier by receptor-mediated transcytosis. Brain uptake of the bispecific antibody can be further enhanced by engineering the bi-specific antibody to reduce its affinity to the blood brain barrier receptor. Reduced affinity for the receptor resulted in a broader distribution in the brain (see, e.g., Atwal et al., Sci. Trans. Med. 3, 84ra43, 2011; Yu et al., Sci. Trans. Med. 3, 84ra44, 2011).

Exemplary bispecific antibodies can also be: (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; or (5) a so-called Scorpion molecule, comprising, e.g., two scFvs fused to both termini of a human Fc-region. Examples of platforms useful for preparing bispecific antibodies include BiTE (Micromet), DART (MacroGenics), Fcab and Mab2 (F-star), Fc-engineered IgG1 (Xencor) or DuoBody (based on Fab arm exchange, Genmab).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% as measured in a competitive binding assay. Some test antibodies inhibit binding of the references antibody by at least 75%, 90% or 99%. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “pharmaceutically acceptable” means that the carrier, diluent, excipient, or auxiliary is compatible with the other ingredients of the formulation and not substantially deleterious to the recipient thereof.

The term “patient” includes human and other mammalian subjects that receive either prophylactic or therapeutic treatment.

An individual is at increased risk of a disease if the subject has at least one known risk-factor (e.g., genetic, biochemical, family history, and situational exposure) placing individuals with that risk factor at a statistically significant greater risk of developing the disease than individuals without the risk factor.

The term “biological sample” refers to a sample of biological material within or obtainable from a biological source, for example a human or mammalian subject. Such samples can be organs, organelles, tissues, sections of tissues, bodily fluids, peripheral blood, blood plasma, blood serum, cells, molecules such as proteins and peptides, and any parts or combinations derived therefrom. The term biological sample can also encompass any material derived by processing the sample. Derived material can include cells or their progeny. Processing of the biological sample may involve one or more of filtration, distillation, extraction, concentration, fixation, inactivation of interfering components, and the like.

The term “control sample” refers to a biological sample not known or suspected to include tau-related disease-affected regions, or at least not known or suspect to include diseased regions of a given type. Control samples can be obtained from individuals not afflicted with the tau-related disease. Alternatively, control samples can be obtained from patients afflicted with the tau-related disease. Such samples can be obtained at the same time as a biological sample thought to comprise the tau-related disease or on a different occasion. A biological sample and a control sample can both be obtained from the same tissue. Preferably, control samples consist essentially or entirely of normal, healthy regions and can be used in comparison to a biological sample thought to comprise tau-related disease-affected regions. Preferably, the tissue in the control sample is the same type as the tissue in the biological sample. Preferably, the tau-related disease-affected cells thought to be in the biological sample arise from the same cell type (e.g., neurons or glia) as the type of cells in the control sample.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” refers to objective evidence of a disease as observed by a physician.

The term “positive response to treatment” refers to a more favorable response in an individual patient or average response in a population of patients relative to an average response in a control population not receiving treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” or “including” one or more recited elements may include other elements not specifically recited. For example, a composition that “comprises” or “includes” an antibody may contain the antibody alone or in combination with other ingredients.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value.

Statistical significance means p≤0.05.

The singular forms of the articles “a,” “an,” and “the” include plural references unless the context clearly dictates otherwise. For example, the term “a compound” or “at least one compound” can include a plurality of compounds, including mixtures thereof.

### DETAILED DESCRIPTION

### I. General

The invention provides antibodies that bind to tau. Some antibodies specifically bind to an epitope within (Q/E)IVYK(S/P) (SEQ ID NO:56). Some antibodies specifically bind to a peptide comprising amino acid sequence QIVYKP (SEQ ID NO:57, corresponding to residues 307-312 of the tau isoform of SEQ ID NO:1). Some antibodies specifically bind to a peptide comprising amino acid sequence EIVYKSP (SEQ ID NO:58, corresponding to residues 391-397 of the tau isoform of SEQ ID NO:1). The antibodies differ from 3D6 and other antibodies characterized in binding to the microtubule binding region (MTBR) of human tau in having an additional epitope near the C-terminus of tau. The epitope's additional C-terminal specificity provides a basis for an antibody to bind an increased number of conformational forms of tau associated with pathology. Some antibodies specifically bind to a peptide comprising amino acid sequence EIVYKS (SEQ ID NO:277, corresponding to residues 391-396 of the tau isoform of SEQ ID NO:1). Exemplary antibodies of the invention are 9F5, 10C12, 2D11, 12C4, 17C12, and 14H3. Some antibodies of the invention serve to inhibit or delay tau-associated pathologies and associated symptomatic deterioration. Although an understanding of mechanism is not required for practice of the invention, a reduction in toxicity may occur as a result of the antibody inducing phagocytosis of tau, inhibiting tau from inter or intramolecular aggregation, or from binding to other molecules, by stabilizing a non-toxic conformation, by inhibiting intercellular or intracellular transmission of pathogenic tau forms, by blockade of tau phosphorylation, by preventing binding of tau to cells, or by inducing proteolytic cleavage of tau, among other mechanisms. Some antibodies of the invention are useful in increasing aggregation of tau, by increasing the molecular weight of certain aggregated tau species to decrease toxicity/cell uptake and/or increase clearance. Large aggregates of tau molecules may exhibit reduced uptake into neuronal cells. Some antibodies of the invention, by bivalent binding to tau, bring separate tau molecules into closer proximity, encouraging aggregation into a tau aggregate too large for uptake into neuronal cells. In addition, Fc-mediated phagocytosis requires several tau-binding antibodies to be in close proximity. A large aggregate of tau molecules may have many anti-tau antibodies bound to it and provide the cluster needed for Fc-mediated phagocytosis by the macrophage. The antibodies of the invention or agents that induce such antibodies can be used in methods of treating or effecting prophylaxis of Alzheimer's and other diseases associated with tau.

### II. Target Molecules

Unless otherwise apparent from the context, reference to tau means a natural human form of tau including all isoforms irrespective of whether posttranslational modification (e.g., phosphorylation, glycation, or acetylation) is present. There are six major isoforms (splice variants) of tau occurring in the human brain. The longest of these variants has 441 amino acids, of which the initial met residue is cleaved. Residues are numbered according to the 441 isoform. Thus, for example, reference to a phosphorylation at position 404 means position 404 of the 441 isoform, or corresponding position of any other isoform when maximally aligned with the 441 isoform. The amino acid sequences of the isoforms and Swiss-Prot numbers are indicated below.

```
P10636-8
(SEQ ID NO: 1)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEDVTAPLV DEGAPGKQAA AQPHTEIPEG TTAEEAGIGD TPSLEDEAAG
              130 140 150 160 170 180
HVTQARMVSK SKDGTGSDDK KAKGADGKTK IATPRGAAPP GQKGQANATR IPAKTPPAPK
              190 200 210 220 230 240
TPPSSGEPPK SGDRSGYSSP GSPGTPGSRS RTPSLPTPPT REPKKVAVVR TPPKSPSSAK
              250 260 270 280 290 300
SRLQTAPVPM PDLKNVKSKI GSTENLKHQP GGGKVQIINK KLDLSNVQSK CGSKDNIKHV
              310 320 330 340 350 360
PGGGSVQIVY KPVDLSKVTS KCGSLGNIHH KPGGGQVEVK SEKLDFKDRV QSKIGSLDNI
              370 380 390 400 410 420
THVPGGGNKK IETHKLTFRE NAKAKTDHGA EIVYKSPVVS GDTSPRHLSN VSSTGSIDMV
              430 440
DSPQLATLAD EVSASLAKQG L
P10636-7
(SEQ ID NO: 2)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEAEEAGIG DTPSLEDEAA GHVTQARMVS KSKDGTGSDD KKAKGADGKT
              130 140 150 160 170 180
KIATPRGAAP PGQKGQANAT RIPAKTPPAP KTPPSSGEPP KSGDRSGYSS PGSPGTPGSR
              190 200 210 220 230 240
SRTPSLPTPP TREPKKVAVV RTPPKSPSSA KSRLQTAPVP MPDLKNVKSK IGSTENLKHQ
              250 260 270 280 290 300
PGGGKVQIIN KKLDLSNVQS KCGSKDNIKH VPGGGSVQIV YKPVDLSKVT SKCGSLGNIH
              310 320 330 340 350 360
HKPGGGQVEV KSEKLDFKDR VQSKIGSLDN ITHVPGGGNK KIETHKLTFR ENAKAKTDHG
              370 380 390 400 410
AEIVYKSPVV SGDTSPRHLS NVSSTGSIDM VDSPQLATLA DEVSASLAKQ GL
P10636-6 (4R0N human tau)
(SEQ ID NO: 3)
                10 20 30 40 50 60
MAEPRQEFEV MEDHAGTYGL GDRKDQGGYT MHQDQEGDTD AGLKAEEAGI GDTPSLEDEA
                70 80 90 100 110 120
AGHVTQARMV SKSKDGTGSD DKKAKGADGK TKIATPRGAA PPGQKGQANA TRIPAKTPPA
              130 140 150 160 170 180
PKTPPSSGEP PKSGDRSGYS SPGSPGTPGS RSRTPSLPTP PTREPKKVAV VRTPPKSPSS
              190 200 210 220 230 240
AKSRLQTAPV PMPDLKNVKS KIGSTENLKH QPGGGKVQII NKKLDLSNVQ SKCGSKDNIK
              250 260 270 280 290 300
HVPGGGSVQI VYKPVDLSKV TSKCGSLGNI HHKPGGGQVE VKSEKLDFKD RVQSKIGSLD
              310 320 330 340 350 360
NITHVPGGGN KKIETHKLTF RENAKAKTDH GAEIVYKSPV VSGDTSPRHL SNVSSTGSID
              370 380
MVDSPQLATL ADEVSASLAK QGL
P10636-5
(SEQ ID NO: 4)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEDVTAPLV DEGAPGKQAA AQPHTEIPEG TTAEEAGIGD TPSLEDEAAG
              130 140 150 160 170 180
HVTQARMVSK SKDGTGSDDK KAKGADGKTK IATPRGAAPP GQKGQANATR IPAKTPPAPK
              190 200 210 220 230 240
TPPSSGEPPK SGDRSGYSSP GSPGTPGSRS RTPSLPTPPT REPKKVAVVR TPPKSPSSAK
              250 260 270 280 290 300
SRLQTAPVPM PDLKNVKSKI GSTENLKHQP GGGKVQIVYK PVDLSKVTSK CGSLGNIHHK
              310 320 330 340 350 360
PGGGQVEVKS EKLDFKDRVQ SKIGSLDNIT HVPGGGNKKI ETHKLTFREN AKAKTDHGAE
              370 380 390 400 410
IVYKSPVVSG DTSPRHLSNV SSTGSIDMVD SPQLATLADE VSASLAKQGL
P10636-4
(SEQ ID NO: 5)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEAEEAGIG DTPSLEDEAA GHVTQARMVS KSKDGTGSDD KKAKGADGKT
              130 140 150 160 170 180
KIATPRGAAP PGQKGQANAT RIPAKTPPAP KTPPSSGEPP KSGDRSGYSS PGSPGTPGSR
              190 200 210 220 230 240
SRTPSLPTPP TREPKKVAVV RTPPKSPSSA KSRLQTAPVP MPDLKNVKSK IGSTENLKHQ
              250 260 270 280 290 300
PGGGKVQIVY KPVDLSKVTS KCGSLGNIHH KPGGGQVEVK SEKLDFKDRV QSKIGSLDNI
              310 320 330 340 350 360
THVPGGGNKK IETHKLTFRE NAKAKTDHGA EIVYKSPVVS GDTSPRHLSN VSSTGSIDMV
              370 380
DSPQLATLAD EVSASLAKQG L
P10636-2
(SEQ ID NO: 6)
                10 20 30 40 50 60
MAEPRQEFEV MEDHAGTYGL GDRKDQGGYT MHQDQEGDTD AGLKAEEAGI GDTPSLEDEA
                70 80 90 100 110 120
AGHVTQARMV SKSKDGTGSD DKKAKGADGK TKIATPRGAA PPGQKGQANA TRIPAKTPPA
              130 140 150 160 170 180
PKTPPSSGEP PKSGDRSGYS SPGSPGTPGS RSRTPSLPTP PTREPKKVAV VRTPPKSPSS
              190 200 210 220 230 240
AKSRLQTAPV PMPDLKNVKS KIGSTENLKH QPGGGKVQIV YKPVDLSKVT SKCGSLGNIH
              250 260 270 280 290 300
HKPGGGQVEV KSEKLDFKDR VQSKIGSLDN ITHVPGGGNK KIETHKLTFR ENAKAKTDHG
              310 320 330 340 350
AEIVYKSPVV SGDTSPRHLS NVSSTGSIDM VDSPQLATLA DEVSASLAKQ GL
```

Reference to tau includes known natural variations about 30 of which are listed in the Swiss-Prot database and permutations thereof, as well as mutations associated with tau pathologies, such as dementia, Pick's disease, supranuclear palsy, etc. (see, e.g., Swiss-Prot database and Poorkaj, et al. Ann Neurol. 43:815-825 (1998)). Some examples of tau mutations numbered by the 441 isoform are a lysine to threonine mutation at amino acid residue 257 (K257T), an isoleucine to valine mutation at amino acid position 260 (I260V); a glycine to valine mutation at amino acid position 272 (G272V); an asparagine to lysine mutation at amino acid position 279 (N279K); an asparagine to histidine mutation at amino acid position 296 (N296H); a proline to serine mutation at amino acid position 301 (P301S); a proline to leucine mutation at amino acid 301 (P301L); a glycine to valine mutation at amino acid position 303 (G303V); a serine to asparagine mutation at position 305 (S305N); a glycine to serine mutation at amino acid position 335 (G335S); a valine to methionine mutation at position 337 (V337M); a glutamic acid to valine mutation at position 342 (E342V); a lysine to isoleucine mutation at amino acid position 369 (K3691); a glycine to arginine mutation at amino acid position 389 (G389R); and an arginine to tryptophan mutation at amino acid position 406 (R406W).

Tau can be phosphorylated at one or more amino acid residues including tyrosine at amino acid positions 18, 29, 97, 310, and 394 serine at amino acid positions 184, 185, 198, 199, 202, 208, 214, 235, 237, 238, 262, 293, 324, 356, 396, 400, 404, 409, 412, 413, and 422; and threonine at amino acids positions 175, 181, 205, 212, 217, 231, and 403.

Unless otherwise apparent from context, reference to tau, or their fragments includes the natural human amino acid sequences including isoforms, mutants, and allelic variants thereof.

### III. Antibodies

A. Binding Specificity and Functional Properties

The invention provides antibodies that specifically bind to tau. Some antibodies specifically bind to tau at an epitope formed by amino acids from either or both of two regions of tau having a common core motif of IVYK (SEQ ID NO:276). These regions are defined by residues 307-312 and 391-397 or 391-396 of SEQ ID NO:1 respectively. Thus, an antibody with one binding site for tau, such as an scFv can specifically bind to tau at an epitope formed from amino acids within either of these regions individually or to a hybrid epitope formed by amino acids from both these regions. An antibody with two binding sites for tau can also bind epitopes within 307-312 and 391-397 or within 391-396 simultaneously from its two binding sites. The epitope can be on the same or different molecules of tau. Some antibodies of the invention specifically bind to a peptide consisting of residues 307-312 of tau, namely residues QIVYKP (SEQ ID NO:57). Some antibodies of the invention specifically bind to a peptide consisting of residues 391-397 of tau, namely EIVYKSP (SEQ ID NO:58). Some antibodies of the invention specifically bind to a peptide consisting of residues 391-396 of tau, namely EIVYKS (SEQ ID NO:277). Some antibodies of the invention specifically bind to a peptide consisting of the consensus motif (Q/E)IVYK(S/P) (SEQ ID NO:56). These antibodies can be obtained by immunizing with a tau polypeptide purified from a natural source or recombinantly expressed. Antibodies can be screened for binding tau in unphosphorylated form as well as a form in which one or more residues susceptible to phosphorylation are phosphorylated. The invention also provides antibodies binding to the same epitope as any of the foregoing antibodies, such as, for example, the epitope of 9F5, 10C12, 2D11, 12C4, 17C12, or 14H3. Also included are antibodies competing for binding to tau with any of the foregoing antibodies, such as, for example, competing with 9F5, 10C12, 2D11, 12C4, 17C12, or 14H3. In an embodiment, antibodies binding to the same epitope as a reference antibody such as 9F5 or competing with the reference antibody share one or more of its functional properties, such as capacity to inhibit tau internalization into neuronal cells. Optionally, such property is possessed to the same extent within experimental error, or greater than that of the reference antibody.

The invention provides an antibody that competes with 9F5 for binding to tau and reduces tau-induced toxicity in neurons. The invention provides an antibody that competes with 9F5 for binding to tau and has increased resistance to agitation stress. Exemplary 9F5 humanized antibodies that compete with 9F5 for binding to tau and have increased resistance to agitation stress are L27cS/L37Q/M51G/L54R (DIM2), also known as hu9F5VHv9/hu9F5VLv8_DIM2 SEQ ID NO: 127/SEQ ID NO: 133, L27cG/L37G/M51G/L54T (DIM14), also known as hu9F5VHv9/hu9F5VLv8_DIM14 SEQ ID NO: 127/SEQ ID NO: 145, and L27cG/L37G/M51G/L54R (DIM13), also known as hu9F5VHv9/hu9F5VLv8_DIM13 SEQ ID NO: 127/SEQ ID NO: 144. The invention provides an antibody that competes with 9F5 for binding to tau and has increased resistance to low pH stress. Exemplary 9F5 humanized antibodies that compete with 9F5 for binding to tau and have increased resistance to low pH stress are L27cS/L37Q/M51G/L54R (DIM2), also known as hu9F5VHv9/hu9F5VLv8_DIM2 SEQ ID NO: 127/SEQ ID NO: 133, L27cD/L37Q/M51G/L54R (DIM6), also known as hu9F5VHv9/hu9F5VLv8_DIM6 , SEQ ID NO: 127/SEQ ID NO:137, and L27cG/L37G/M51G/L54R (DIM13), also known as hu9F5VHv9/hu9F5VLv8_DIM13 SEQ ID NO: 127/SEQ ID NO: 144. Exemplary 9F5 humanized antibodies that compete with 9F5 for binding to tau and have increased resistance to agitation stress and have increased resistance to low pH stress are DIM 2 [hu9F5VHv9/hu9F5VLv8_DIM2 SEQ ID NO: 127/SEQ ID NO: 133], DIM6 [hu9F5VHv9/hu9F5VLv8_DIM6 SEQ ID NO: 127/SEQ ID NO:137], DIM7 [hu9F5VHv9/hu9F5VLv8_DIM7 SEQ ID NO: 127/SEQ ID NO: 138], DIM8 [hu9F5VHv9/hu9F5VLv8_DIM8 SEQ ID NO: 127/SEQ ID NO: 139], DIM13 [hu9F5VHv9/hu9F5VLv8_DIM13 SEQ ID NO: 127/SEQ ID NO: 144], DIM18 [hu9F5VHv9/hu9F5VLv8_DIM18 SEQ ID NO: 127/SEQ ID NO: 149], DIM28 [hu9F5VHv9/hu9F5VLv8_DIM28 SEQ ID NO: 127/SEQ ID NO: 159, and DIM30 [hu9F5VHv9/hu9F5VLv8_DIM30 SEQ ID NO: 127/SEQ ID NO: 161].

The invention provides an antibody that competes with 9F5 for binding to tau and has reduced propensity to aggregate under high concentration conditions. An exemplary antibody that competes with 9F5 for binding to tau and has reduced propensity to aggregate under high concentration conditions is L37Q/M51G/L54R combined with the original leucine at position L27c(DIM27), also known as hu9F5VHv9/hu9F5VLv8_DIM27 SEQ ID NO: 127/SEQ ID NO: 158.

The above-mentioned antibodies can be generated de novo by immunizing with a tau peptide comprising or consisting of amino acid sequence QIVYKP (SEQ ID NO:57), comprising amino acid sequence EIVYKSP (SEQ ID NO:58), comprising amino acid sequence EIVYKS (SEQ ID NO:277) or comprising or consisting of amino acid sequence (Q/E)IVYK(S/P) (SEQ ID NO:56) or by immunizing with a full length tau polypeptide or fragment thereof comprising such residues and screening for specific binding to a peptide including such residues. Such tau peptides are preferably attached to a heterologous conjugate molecule that helps elicit an antibody response to the peptide. Attachment can be direct or via a spacer peptide or amino acid. Cysteine is used as a spacer amino acid because its free SH group facilitates attachment of a carrier molecule. A polyglycine linker (e.g., 2-6 glycines), with or without a cysteine residue between the glycines and the peptide can also be used. The carrier molecule serves to provide a T-cell epitope that helps elicit an antibody response against the peptide. Several carriers are commonly used particularly keyhole limpet hemocyanin (KLH), ovalbumin and bovine serum albumin (BSA). Peptide spacers can be added to peptide immunogen as part of solid phase peptide synthesis. Carriers are typically added by chemical cross-linking. Some examples of chemical crosslinkers that can be used include cross-N-maleimido-6-aminocaproyl ester or m-maleimidobenzoyl-N-hydroxysuccinimide ester (MBS) (see for example, Harlow, E. et al., Antibodies: A Laboratory Manual, Cold Spring Harbor Laboratory Press, Cold Spring Harbor, N.Y. 1988; Sinigaglia et al., Nature, 336:778-780 (1988); Chicz et al., J. Exp. Med., 178:27-47 (1993); Hammer et al., Cell 74:197-203 (1993); Falk K. et al., Immunogenetics, 39:230-242 (1994); WO 98/23635; and, Southwood et al. J. Immunology, 160:3363-3373 (1998)). The carrier and spacer if present can be attached to either end of the immunogen.

A peptide with optional spacer and carrier can be used to immunize laboratory animals or B-cells as described in more detail below. Hybridoma supernatants can be tested for ability to bind a tau peptide comprising or consisting of amino acid sequence QIVYKP (SEQ ID NO:57), a peptide comprising or consisting of amino acid sequence EIVYKSP (SEQ ID NO:58), a peptide comprising or consisting of amino acid sequence EIVYKS (SEQ ID NO:277),or a peptide comprising or consisting of the amino acid sequence (Q/E)IVYK(S/P) (SEQ ID NO:56) and/or phosphorylated and non-phosphorylated forms of tau, such as, for example, a full-length isoform of tau with position 404 in phosphorylated form. The peptide can be attached to a carrier or other tag to facilitate the screening assay. In this case, the carrier or tag is preferentially different than the combination of spacer and carrier molecule used for immunization to eliminate antibodies specific for the spacer or carrier rather than the tau peptide. Any of the tau isoforms can be used.

An antibody designated 9F5 is an exemplary antibody specifically binding to tau. Unless otherwise apparent from context, reference to 9F5 should be understood as referring to any of the mouse, chimeric, veneered, and humanized forms of this antibody. The antibody has been deposited as [DEPOSIT NUMBER]. This antibody specifically binds a peptide comprising or consisting of amino acid sequence QIVYKP (SEQ ID NO:57), a peptide comprising or consisting of amino acid sequence EIVYKSP (SEQ ID NO:58), or a peptide comprising or consisting of amino acid sequence (Q/E)IVYK(S/P) (SEQ ID NO:56). Kabat/Chothia Composite CDRs of the heavy chain of 9F5 are designated SEQ ID NOs:8, 9, and 10, respectively, and Kabat CDRs of the light chain of 9F5 are designated SEQ ID NOs:12, 13, and 14, respectively.

Additional antibodies that compete with 9F5 for binding to tau and/or bind the same or overlapping epitope as 9F5 have been isolated designated 10C12, 2D11, 12C4, 17C12, and 14H3 and produced by hybridomas of the same names. 10C12 has mature variable heavy and light regions (after cleavage of signal peptide) characterized by SEQ ID NO:7 and SEQ ID NO:11 respectively. Unless otherwise apparent from the context, reference to 10C12 should be understood as referring to any of the mouse, chimeric, veneered, and humanized forms of this antibody. 10C12 has been deposited as [DEPOSIT NUMBER]. 10C12 is further characterized by its ability to bind both non-pathological and pathological forms and conformations of tau, and misfolded/aggregated forms of tau. 10C12 binds structural features such as tau tangles and dystrophic neurites in tissue from Alzheimer's disease, and precipitates both monomeric and aggregated tau from Alzheimer's disease extracts.

2D11 has mature variable heavy and light regions (after cleavage of signal peptide) characterized by SEQ ID NO:7 and SEQ ID NO:11 respectively. Unless otherwise apparent from the context, reference to 2D11 should be understood as referring to any of the mouse, chimeric, veneered, and humanized forms of this antibody. 2D11 has been deposited as [DEPOSIT NUMBER]. 2D11 is further characterized by its ability to bind both non-pathological and pathological forms and conformations of tau, and misfolded/aggregated forms of tau. 2D11 binds structural features such as tau tangles and dystrophic neurites in tissue from Alzheimer's disease, and precipitates both monomeric and aggregated tau from Alzheimer's disease extracts.

12C4 has mature variable heavy and light regions (after cleavage of signal peptide) characterized by SEQ ID NO:219 and SEQ ID NO:11 respectively. Unless otherwise apparent from the context, reference to 12C4 should be understood as referring to any of the mouse, chimeric, veneered, and humanized forms of this antibody. 12C4 has been deposited as [DEPOSIT NUMBER]. 12C4 is further characterized by its ability to bind both non-pathological and pathological forms and conformations of tau, and misfolded/aggregated forms of tau. 12C4 binds structural features such as tau tangles and dystrophic neurites in tissue from Alzheimer's disease, and precipitates both monomeric and aggregated tau from Alzheimer's disease extracts.

17C12 has mature variable heavy and light regions (after cleavage of signal peptide) characterized by SEQ ID NO:225 and SEQ ID NO:228 respectively. Unless otherwise apparent from the context, reference to 17C12 should be understood as referring to any of the mouse, chimeric, veneered, and humanized forms of this antibody. 17C12 has been deposited as [DEPOSIT NUMBER]. 17C12 is further characterized by its ability to bind both non-pathological and pathological forms and conformations of tau, and misfolded/aggregated forms of tau.

14H3 has mature variable heavy and light regions (after cleavage of signal peptide) characterized by SEQ ID NO:240 and SEQ ID NO:244 respectively. Unless otherwise apparent from the context, reference to 14H3 should be understood as referring to any of the mouse, chimeric, veneered, and humanized forms of this antibody. 14H3 has been deposited as [DEPOSIT NUMBER]. 14H3 is further characterized by its ability to bind both non-pathological and pathological forms and conformations of tau, and misfolded/aggregated forms of tau. 14H3 binds structural features such as tau tangles and dystrophic neurites in tissue from Alzheimer's disease.

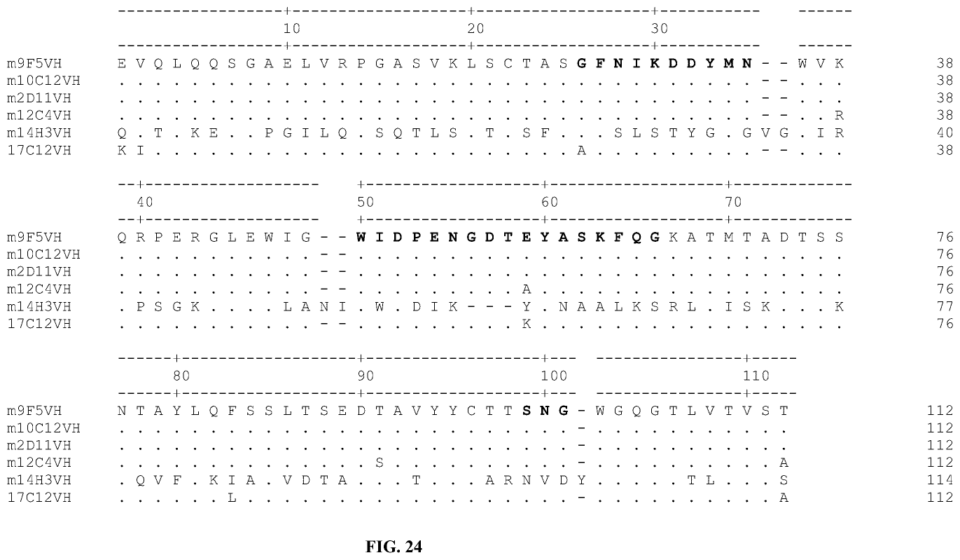

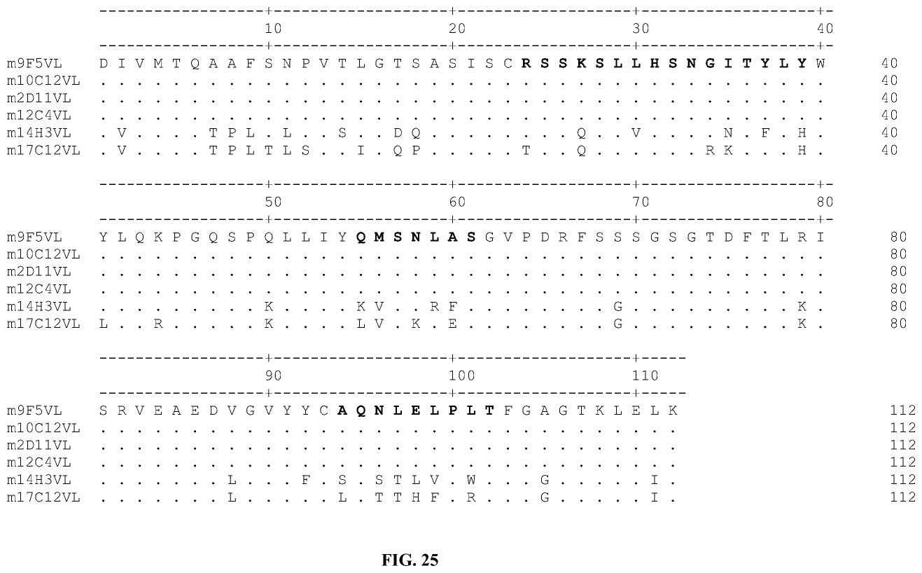

Alignments of the mature heavy chain variable regions of mouse 9F5, 10C12, 2D11, 12C4, 14H3, and 17C12 antibodies are depicted in FIG. 24, and alignments of the mature light chain variable regions of mouse 9F5, 10C12, 2D11, 12C4, 14H3, and 17C12 antibodies are depicted in FIG. 25. The amino acid sequence of the mature heavy chain variable region of mouse 10C12 antibody has 100% sequence identity to that of mouse 9F5 antibody, and the mature light chain variable region of mouse 10C12 antibody has 100% sequence identity to that of mouse 9F5 antibody. The amino acid sequence of the mature heavy chain variable region of mouse 2D11 antibody has 100% sequence identity to that of mouse 9F5 antibody, and the mature light chain variable region of mouse 2D11 antibody has 100% sequence identity to that of mouse 9F5 antibody. The amino acid sequence of the mature heavy chain variable region of mouse 12C4 antibody has 96.6% sequence identity to that of mouse 9F5 antibody, and the mature light chain variable region of mouse 12C4 antibody has 100% sequence identity to that of mouse 9F5 antibody. The amino acid sequence of the mature heavy chain variable region of mouse 17C12 antibody has 95.9% sequence identity to that of mouse 9F5 antibody, and the mature light chain variable region of mouse 17C12 antibody has 70.5% sequence identity to that of mouse 9F5 antibody. The amino acid sequence of the mature heavy chain variable region of mouse 14H3 antibody has 35.0% sequence identity to that of mouse 9F5 antibody, and the mature light chain variable region of mouse 14H3 antibody has 73.2% sequence identity to that of mouse 9F5 antibody.

Optionally, the antibodies of the invention do not include a 10C12 antibody. Optionally, the antibodies of the invention do not include a 2D11 antibody. Optionally, the antibodies of the invention do not include an 12C4 antibody. Optionally, the antibodies of the invention do not include a 17C12 antibody. Optionally, the antibodies of the invention do not include a 14H3 antibody.

Some antibodies of the invention bind to the same or overlapping epitope as an antibody designated 9F5, 10C12, 2D11, 12C4, 17C12, or 14H3. The sequences of the heavy and light chain mature variable regions of this 9F5 are designated SEQ ID NOs:7 and 11, respectively. The sequences of the heavy and light chain mature variable regions of 10C12 are designated SEQ ID NOs:7 and 11, respectively. The sequences of the heavy and light chain mature variable regions of 2D11 are designated SEQ ID NOs:7 and 11, respectively. The sequences of the heavy and light chain mature variable regions of 12C4 are designated SEQ ID NOs:219 and 11, respectively. The sequences of the heavy and light chain mature variable regions of 17C12 are designated SEQ ID NOs:225 and 228, respectively. The sequences of the heavy and light chain mature variable regions of 14H3 are designated SEQ ID NOs:240 and 244, respectively. Other antibodies having such a binding specificity can be produced by immunizing mice with tau or a portion thereof including the desired epitope (e.g. a tau peptide comprising or consisting of the amino acid sequence QIVYKP (SEQ ID NO:57), a tau peptide comprising or consisting of the amino acid sequence EIVYKSP (SEQ ID NO:58), a tau peptide comprising or consisting of the amino acid sequence EIVYKS (SEQ ID NO:277),or a tau peptide comprising or consisting of the amino acid sequence (Q/E)IVYK(S/P) (SEQ ID NO:56)) and screening resulting antibodies for binding to tau optionally in competition with an antibody having the variable regions of mouse 9F5 (IgG1/kappa), 10C12 (IgG2a/kappa), 2D11 (IgG2a/kappa), 12C4 (IgG2a/kappa), 17C12 (IgG2a/kappa), or 14H3 (IgG2a/kappa). Fragments of tau including the desired epitope can be linked to a carrier that helps elicit an antibody response to the fragment and/or be combined with an adjuvant the helps elicit such a response. Such antibodies can be screened for differential binding to tau or a fragment thereof compared with mutants of specified residues. Screening against such mutants more precisely defines the binding specificity to allow identification of antibodies whose binding is inhibited by mutagenesis of particular residues and which are likely to share the functional properties of other exemplified antibodies. The mutations can be systematic replacement substitution with alanine (or serine if an alanine is present already) one residue at a time, or more broadly spaced intervals, throughout the target or throughout a section thereof in which an epitope is known to reside. If the same set of mutations significantly reduces the binding of two antibodies, the two antibodies bind the same epitope.

Antibodies having the binding specificity of a selected murine antibody (e.g., 9F5, 10C12, 2D11, 12C4, 17C12, or 14H3) can also be produced using a variant of the phage display method. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for tau or a fragment thereof (e.g., at least 10⁸ and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for tau or a fragment thereof are selected. The resulting antibodies usually have the same or similar epitope specificity as the murine starting material.

Kabat/Chothia Composite CDRs of the heavy chain of 9F5 are designated SEQ ID NOs:8-10, respectively, and Kabat CDRs of the light chain of 9F5 are designated SEQ ID NOs: 12-14, respectively.

Kabat/Chothia Composite CDRs of the heavy chain of 10C12 are designated SEQ ID NOs:8-10, respectively, and Kabat CDRs of the light chain of 10C12 are designated SEQ ID NOs: 12-14, respectively.

Kabat/Chothia Composite CDRs of the heavy chain of 2D11 are designated SEQ ID NOs:8-10, respectively, and Kabat CDRs of the light chain of 2D11 are designated SEQ ID NOs: 12-14, respectively.

Table 2 indicates the 9F5, 10C12, and 2D11 CDRs as defined by Kabat, Chothia, Composite of Chothia and Kabat (also referred to herein as “Kabat/Chothia Composite”), AbM, and Contact.

TABLE 2
9F5, 10C12, and 2D11 CDRs as defined by Kabat, Chothia,
Composite of Chothia and Kabat, AbM, and Contact

| | | | Composite of | | |
|---|---|---|---|---|---|
| Loop | Kabat | Chothia | Chothia & Kabat | AbM | Contact |
| L1 | L24--L34 | L24--L34 | L24--L34 | L24--L34 | L30--L36 |
| | SEQ ID NO: 12 | SEQ ID NO: 12 | SEQ ID NO: 12 | SEQ ID NO: 12 | SEQ ID NO: 47 |
| L2 | L50--L56 | L50--L56 | L50--L56 | L50--L56 | L46--L55 |
| | SEQ ID NO: 13 | SEQ ID NO: 13 | SEQ ID NO: 13 | SEQ ID NO: 13 | SEQ ID NO: 48 |
| L3 | L89--L97 | L89--L97 | L89--L97 | L89--L97 | L89--L96 |
| | SEQ ID NO: 14 | SEQ ID NO: 14 | SEQ ID NO: 14 | SEQ ID NO: 14 | SEQ ID NO: 49 |
| H1 | H31--H35B | H26--H32 | H26--H35B | H26--H35B | H30--H35B |
| | SEQ ID NO: 40 | SEQ ID NO: 41 | SEQ ID NO: 8 | SEQ ID NO: 8 | SEQ ID NO: 44 |
| H2 | H50--H65 | H52--H56 | H50--H65 | H50--H58 | H47--H58 |
| | SEQ ID NO: 9 | SEQ ID NO: 42 | SEQ ID NO: 9 | SEQ ID NO: 43 | SEQ ID NO: 45 |
| H3 | H95--H102 | H95--H102 | H95--H102 | H95--H102 | H93--H101 |
| | SEQ ID NO: 10 | SEQ ID NO: 10 | SEQ ID NO: 10 | SEQ ID NO: 10 | SEQ ID NO: 46 |

Kabat/Chothia Composite CDRs of the heavy chain of 12C4 are designated SEQ ID NOs:8, 220, and 10, respectively, and Kabat CDRs of the light chain of 12C4 are designated SEQ ID NOs:12-14, respectively.

Table 3 indicates the 12C4 CDRs as defined by Kabat, Chothia, Composite of Chothia and Kabat (also referred to herein as “Kabat/Chothia Composite”), AbM, and Contact.

TABLE 3
12C4 CDRs as defined by Kabat, Chothia, Composite of Chothia and Kabat, AbM, and Contact

| | | | Composite of | | |
|---|---|---|---|---|---|
| Loop | Kabat | Chothia | Chothia & Kabat | AbM | Contact |
| L1 | L24--L34 | L24--L34 | L24--L34 | L24--L34 | L30--L36 |
| | SEQ ID NO: 12 | SEQ ID NO: 12 | SEQ ID NO: 12 | SEQ ID NO: 12 | SEQ ID NO: 47 |
| L2 | L50--L56 | L50--L56 | L50--L56 | L50--L56 | L46--L55 |
| | SEQ ID NO: 13 | SEQ ID NO: 13 | SEQ ID NO: 13 | SEQ ID NO: 13 | SEQ ID NO: 48 |
| L3 | L89--L97 | L89--L97 | L89--L97 | L89--L97 | L89--L96 |
| | SEQ ID NO: 14 | SEQ ID NO: 14 | SEQ ID NO: 14 | SEQ ID NO: 14 | SEQ ID NO: 49 |
| H1 | H31--H35B | H26--H32 | H26--H35B | H26--H35B | H30--H35B |
| | SEQ ID NO: 40 | SEQ ID NO: 41 | SEQ ID NO: 8 | SEQ ID NO: 8 | SEQ ID NO: 44 |
| H2 | H50--H65 | H52--H56 | H50--H65 | H50--H58 | H47--H58 |
| | SEQ ID NO: 220 | SEQ ID NO: 42 | SEQ ID NO: 220 | SEQ ID NO: 257 | SEQ ID NO: 258 |
| H3 | H95--H102 | H95--H102 | H95--H102 | H95--H102 | H93--H101 |
| | SEQ ID NO: 10 | SEQ ID NO: 10 | SEQ ID NO: 10 | SEQ ID NO: 10 | SEQ ID NO: 46 |

Kabat/Chothia Composite CDRs of the heavy chain of 17C12 are designated SEQ ID NOs:226, 227, and 10, respectively, and Kabat CDRs of the light chain of 17C12 are designated SEQ ID NOs:229-231 respectively.

Table 4 indicates the 17C12 CDRs as defined by Kabat, Chothia, Composite of Chothia and Kabat (also referred to herein as “Kabat/Chothia Composite”), AbM, and Contact.

TABLE 4
17C12 CDRs as defined by Kabat, Chothia, Composite of Chothia and Kabat, AbM, and Contact

| | | | Composite of | | |
|---|---|---|---|---|---|
| Loop | Kabat | Chothia | Chothia & Kabat | AbM | Contact |
| L1 | L24--L34 | L24--L34 | L24--L34 | L24--L34 | L30--L36 |
| | SEQ ID NO: 229 | SEQ ID NO: 229 | SEQ ID NO: 229 | SEQ ID NO: 229 | SEQ ID NO: 262 |
| L2 | L50--L56 | L50--L56 | L50--L56 | L50--L56 | L46--L55 |
| | SEQ ID NO: 230 | SEQ ID NO: 230 | SEQ ID NO: 230 | SEQ ID NO: 230 | SEQ ID NO: 263 |
| L3 | L89--L97 | L89--L97 | L89--L97 | L89--L97 | L89--L96 |
| | SEQ ID NO: 231 | SEQ ID NO: 231 | SEQ ID NO: 231 | SEQ ID NO: 231 | SEQ ID NO: 264 |
| H1 | H31--H35B | H26--H32 | H26--H35B | H26--H35B | H30--H35B |
| | SEQ ID NO: 40 | SEQ ID NO: 259 | SEQ ID NO: 226 | SEQ ID NO: 226 | SEQ ID NO: 44 |
| H2 | H50--H65 | H52--H56 | H50--H65 | H50--H58 | H47--H58 |
| | SEQ ID NO: 227 | SEQ ID NO: 42 | SEQ ID NO: 227 | SEQ ID NO: 260 | SEQ ID NO: 261 |
| H3 | H95--H102 | H95--H102 | H95--H102 | H95--H102 | H93--H101 |
| | SEQ ID NO: 10 | SEQ ID NO: 10 | SEQ ID NO: 10 | SEQ ID NO: 10 | SEQ ID NO: 46 |

Kabat/Chothia Composite CDRs of the heavy chain of 14H3 are designated SEQ ID NOs:241-243, respectively, and Kabat CDRs of the light chain of 14H3 are designated SEQ ID NOs:245-247, respectively.

Table 5 indicates the 14H3 CDRs as defined by Kabat, Chothia, Composite of Chothia and Kabat (also referred to herein as “Kabat/Chothia Composite”), AbM, and Contact.

TABLE 5
14H3 CDRs as defined by Kabat, Chothia, Composite of Chothia and Kabat, AbM, and Contact

| | | | Composite of | | |
|---|---|---|---|---|---|
| Loop | Kabat | Chothia | Chothia & Kabat | AbM | Contact |
| L1 | L24--L34 | L24--L34 | L24--L34 | L24--L34 | L30--L36 |
| | SEQ ID NO: 245 | SEQ ID NO: 245 | SEQ ID NO: 245 | SEQ ID NO: 245 | SEQ ID NO: 272 |
| L2 | L50--L56 | L50--L56 | L50--L56 | L50--L56 | L46--L55 |
| | SEQ ID NO: 246 | SEQ ID NO: 246 | SEQ ID NO: 246 | SEQ ID NO: 246 | SEQ ID NO: 273 |
| L3 | L89--L97 | L89--L97 | L89--L97 | L89--L97 | L89--L96 |
| | SEQ ID NO: 247 | SEQ ID NO: 247 | SEQ ID NO: 247 | SEQ ID NO: 247 | SEQ ID NO: 274 |
| H1 | H31--H35B | H26--H32 | H26--H35B | H26--H35B | H30--H35B |
| | SEQ ID NO: 265 | SEQ ID NO: 266 | SEQ ID NO: 241 | SEQ ID NO: 241 | SEQ ID NO: 269 |
| H2 | H50--H65 | H52--H56 | H50--H65 | H50--H58 | H47--H58 |
| | SEQ ID NO: 242 | SEQ ID NO: 267 | SEQ ID NO: 242 | SEQ ID NO: 268 | SEQ ID NO: 270 |
| H3 | H95--H102 | H95--H102 | H95--H102 | H95--H102 | H93--H101 |
| | SEQ ID NO: 243 | SEQ ID NO: 243 | SEQ ID NO: 243 | SEQ ID NO: 243 | SEQ ID NO: 271 |

Other antibodies can be obtained by mutagenesis of cDNA encoding the heavy and light chains of an exemplary antibody, such as 9F5, 10C12, 2D11, 12C4, 17C12, or 14H3. Monoclonal antibodies that are at least 70%, 80%, 90%, 95%, 96%, 97%, 98%, or 99% identical to 9F5, 10C12, 2D11, 12C4, 17C12, or 14H3 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one or all six CDR(s) as defined by any conventional definition, but preferably Kabat, that are 90%, 95%, 99% or 100% identical to corresponding CDRs of 9F5, 10C12, 2D11, 12C4, 17C12, or 14H3 are also included.

The invention also provides antibodies having some or all (e.g., 3, 4, 5, and 6) CDRs entirely or substantially from 9F5, 10C12, 2D11, 12C4, 17C12, or 14H3. Such antibodies can include a heavy chain variable region that has at least two, and usually all three, CDRs entirely or substantially from the heavy chain variable region of 9F5, 10C12, 2D11, 12C4, 17C12, or 14H3 and/or a light chain variable region having at least two, and usually all three, CDRs entirely or substantially from the light chain variable region of 9F5, 10C12, 2D11, 12C4, 17C12, or 14H3. The antibodies can include both heavy and light chains. A CDR is substantially from a corresponding 9F5 CDR when it contains no more than 4, 3, 2, or 1 substitutions, insertions, or deletions, except that CDR-H2 (when defined by Kabat) can have no more than 6, 5, 4, 3, 2, or 1 substitutions, insertions, or deletions. Such antibodies can have at least 70%, 80%, 90%, 95%, 96%, 97%, 98%, or 99% identity to 9F5, 10C12, 2D11, 12C4, 17C12, or 14H3 in the amino acid sequence of the mature heavy and/or light chain variable regions and maintain their functional properties, and/or differ from 9F5, 10C12, 2D11, 12C4, 17C12, or 14H3 by a small number of functionally inconsequential amino acid substitutions (e.g., conservative substitutions), deletions, or insertions.

Some antibodies identified by such assays can bind to monomeric, misfolded, aggregated, phosphorylated, or unphosphorylated forms of tau or otherwise. Likewise, some antibodies are immunoreactive on non-pathological and pathological forms and conformations of tau.

The invention further provides a means for specifically binding to a peptide consisting of residues (Q/E)IVYK(S/P) (SEQ ID NO:56), residues QIVYKP (SEQ ID NO:57) residues EIVYKSP (SEQ ID NO:58), or residues EIVYKS (SEQ ID NO:277). An exemplary means is an antibody comprising the heavy chain CDRs of SEQ ID NOs:8-10 and light chain CDRs of SEQ ID NOs.:12-14. An exemplary means is an antibody comprising the heavy chain CDRs of SEQ ID NOs:8, 220, and 10 and light chain CDRs of SEQ ID NOs:12-14. An exemplary means is an antibody comprising the heavy chain CDRs of SEQ ID NOs:226, 227, and 10 and light chain CDRs of SEQ ID NOs: 229-231. An exemplary means is an antibody comprising the heavy chain CDRs of SEQ ID NOs:241-243 and light chain CDRs of SEQ ID NOs:245-247.

B. Non-Human Antibodies

The production of other non-human antibodies, e.g., murine, guinea pig, primate, rabbit or rat, against tau or a fragment thereof (e.g., a peptide comprising an amino acid sequence of QIVYKP (SEQ ID NO:57), EIVYKSP (SEQ ID NO:58), EIVYKS (SEQ ID NO:277). or (Q/E)IVYK(S/P) (SEQ ID NO:56)) can be accomplished by, for example, immunizing the animal with tau or a fragment thereof. See Harlow & Lane, Antibodies, A Laboratory Manual (CSHP NY, 1988) (incorporated by reference for all purposes). Optionally, the immunogen can be a 383 amino acid human tau (4R0N). Optionally, the immunogen can be a human tau containing a P301S mutation. Optionally, the immunogen can be a human tau, wherein the human tau is recombinant N-terminally His-tagged. Optionally, the animal is immunized with a tau fragment comprising a peptide represented by (Q/E)IVYK(S/P) (SEQ ID NO:56), linked to a carrier. Optionally, the peptide is QIVYKP (SEQ ID NO:57), EIVYKSP (SEQ ID NO:58), or EIVYKS (SEQ ID NO:277). Such an immunogen can be obtained from a natural source, by peptide synthesis, or by recombinant expression. Optionally, the immunogen can be administered fused or otherwise complexed with a carrier protein. Optionally, the immunogen can be administered with an adjuvant. Several types of adjuvant can be used as described below. Complete Freund's adjuvant followed by incomplete adjuvant can be used for immunization of laboratory animals. Rabbits or guinea pigs are typically used for making polyclonal antibodies. Mice are typically used for making monoclonal antibodies. Antibodies are screened for specific binding to tau or an epitope within tau (e.g., QIVYKP (SEQ ID NO:57), EIVYKSP (SEQ ID NO:58), EIVYKS (SEQ ID NO:277). or (Q/E)IVYK(S/P) (SEQ ID NO:56)). Optionally, the screening can be performed against 15 amino acid peptides comprising QIVYKP (SEQ ID NO:57), EIVYKSP (SEQ ID NO:58), EIVYKS (SEQ ID NO:277). or any other consensus motif represented by (Q/E)IVYK(S/P) (SEQ ID NO:56). Optionally, the peptides comprise QIVYKP (SEQ ID NO:57) EIVYKSP (SEQ ID NO:58), EIVYKS (SEQ ID NO:277). Such screening can be accomplished by determining binding of an antibody to a collection of tau variants, such as tau variants comprising or consisting of amino acid residues 307-312 or 391-397 or 391-396 of SEQ ID NO:1) or mutations within these residues, and determining which tau variants bind to the antibody. Binding can be assessed, for example, by Western blot, FACS or ELISA.

C. Humanized Antibodies

A humanized antibody is a genetically engineered antibody in which CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having at least three, four, five or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by any conventional definition but preferably defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical. To be classified as humanized under the 2014 World Health Organization (WHO) International non-proprietary names (INN) definition of humanized antibodies, an antibody must have at least 85% identity to human germline antibody sequences (i.e., prior to somatic hypermutation). Mixed antibodies are antibodies for which one antibody chain (e.g., heavy chain) meets the threshold but the other chain (e.g., light chain) does not meet the threshold. An antibody is classified as chimeric if neither chain meets the threshold, even though the variable framework regions for both chains were substantially human with some murine backmutations. See, Jones et al. (2016) The INNs and outs of antibody nonproprietary names, mAbs 8:1, 1-9, DOI: 10.1080/19420862.2015.1114320. See also “WHO-INN: International nonproprietary names (INN) for biological and biotechnological substances (a review)” (Internet) 2014. Available from: http://www.who.int/medicines/services/inn/BioRev2014.pdf), incorporated herein by reference. For the avoidance of doubt, the term “humanized” as used herein is not intended to be limited to the 2014 WHO INN definition of humanized antibodies. Some of the humanized antibodies provided herein have at least 85% sequence identity to human germline sequences and some of the humanized antibodies provided herein have less than 85% sequence identity to human germline sequences. Some of the heavy chains of the humanized antibodies provided herein have from about 60% to 100% sequence identity to human germ line sequences, such as, for example, in the range of about 60% to 69%, 70% to 79%, 80% to 84%, or 85% to 89%. Some heavy chains fall below the 2014 WHO INN definition and have, for example, about 64%, 65%, 66%, 67%, 68%, 69%, 70%, 71%, 72%, 73%, 74%, 75%, 76%, 77%, 78%, 79%, 80%, 81%, or 82%, 83%, or 84% sequence identity to human germ line sequences, while other heavy chains meet the 2014 WHO INN definition and have about 85%, 86%, 87%, 88%, 89% or greater sequence identity to human germ line sequences. Some of the light chains of the humanized antibodies provided herein have from about 60% to 100% sequence identity to human germ line sequences, such as, for example, in the range of about 80% to 84% or 85% to 89%. Some light chains fall below the 2014 WHO INN definition and have, for example, about 81%, 82%, 83% or 84% sequence identity to human germ line sequences, while other light chains meet the 2014 WHO INN definition and have about 85%, 86%, 87%, 88%, 89% or greater sequence identity to human germ line sequences. Some humanized antibodies provided herein that are “chimeric” under the 2014 WHO INN definition have heavy chains with less than 85% identity to human germ line sequences paired with light chains having less than 85% identity to human germ line sequences. Some humanized antibodies provided herein are “mixed” under the 2014 WHO INN definition, for example, having a heavy chain with at least 85% sequence identity to human germ line sequences paired with a light chain having less than 85% sequence identity to human germ line sequences, or vice versa. Some humanized antibodies provided herein meet the 2014 WHO INN definition of “humanized” and have a heavy chain with at least 85% sequence identity to human germ line sequences paired with a light chain having at least 85% sequence identity to human germ line sequences. Exemplary 12C4 antibodies that meet the 2014 WHO INN definition of “humanized” include antibodies having a mature heavy chain with the amino acid sequence of SEQ ID NO:221 or SEQ ID NO:222 paired with a mature light chain sequence having an amino acid sequence of SEQ ID NO:223 or SEQ ID NO:224. Exemplary 14H3 antibodies that meet the 2014 WHO INN definition of “humanized” include antibodies having a mature heavy chain with the amino acid sequence of SEQ ID NO:248 or SEQ ID NO:249 paired with a mature light chain sequence having an amino acid sequence of SEQ ID NO:251 or SEQ ID NO:252. Some humanized antibodies provided herein meet the 2014 WHO INN definition of “mixed.” Exemplary 9F5 antibodies that meet the 2014 WHO INN definition of “mixed” include antibodies having a mature heavy chain with the amino acid sequence of any of SEQ ID NOs:15-22 and SEQ ID NOs:127-128 paired with a mature light chain sequence having an amino acid sequence of any of SEQ ID NO:26-29 and SEQ ID NOs:130-131. Exemplary 10C12 antibodies that meet the 2014 WHO INN definition of “mixed” include antibodies having a mature heavy chain with the amino acid sequence of SEQ ID NO:214 or SEQ ID NO:215 paired with a mature light chain sequence having an amino acid sequence of SEQ ID NO:216 or SEQ ID NO:217. Exemplary 17C12 antibodies that meet the 2014 WHO INN definition of “mixed” include antibodies having a mature heavy chain with the amino acid sequence of SEQ ID NO:232 or SEQ ID NO:233 paired with a mature light chain sequence having an amino acid sequence of SEQ ID NO:235. Additional humanized 9F5 antibodies of the invention include antibodies having a mature heavy chain having an amino acid sequence of any of SEQ ID NOs:15-22 and SEQ ID NOs:127-128 paired with a mature light chain having an amino acid sequence of any of SEQ ID NOs:23-25. Additional humanized 17C12 antibodies of the invention include antibodies having a mature heavy chain having an amino acid sequence of of SEQ ID NO:232 or SEQ ID NO:233 paired with a mature light chain having an amino acid sequence of SEQ ID NO:234.

Although humanized antibodies often incorporate all six CDRs (defined by any conventional definition but preferably as defined by Kabat) from a mouse antibody, they can also be made with less than all CDRs (e.g., at least 3, 4, or 5 CDRs) from a mouse antibody (e.g., Pascalis et al., J. Immunol. 169:3076, 2002; Vajdos et al., J. of Mol. Biol., 320: 415-428, 2002; Iwahashi et al., Mol. Immunol. 36:1079-1091, 1999; Tamura et al, J. Immunol., 164:1432-1441, 2000).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41: 863, 2004. In such humanized antibodies at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity and/or for meeting the WHO INN definition of “humanized”. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

Some humanized and chimeric antibodies have the same (within experimental error) or improved functional properties, e.g., binding affinity for human tau, inhibition of tau internalization into neurons as described in the examples as a murine antibody from which they were derived. For example, some humanized and chimeric antibodies have a binding affinity within a factor of 3, 2 or 1 of the murine antibody from which they were derived or an affinity indistinguishable within experimental error. Some humanized and chimeric antibodies inhibit tau internalization into neurons as described in the examples within a factor of 3, 2 or 1 of the murine antibody from which they were derived or inhibit the same within experimental error as the mouse antibody from which they were derived.

An example of an acceptor sequence for the 9F5 heavy chain is the human mature heavy chain variable region of humanized 48G7 Fab with PDB accession code 2RCS-VH_huFrwk (SEQ ID NO:32). An example of an acceptor sequence for the 9F5 heavy chain is the human mature heavy chain GenBank AAN16432-VH_huFrwk (SEQ ID NO:31). The variable domains of 9F5, and 48G7 Fab also share identical lengths for the CDR-H1, H2 loops. An example of an acceptor sequence for the 9F5 heavy chain is the human mature heavy chain variable region IMGT# IGHV1-69-2*01 (SEQ ID NO:33). The Chothia CDR-H1 of IMGT# IGHV1-69-2*01 (SEQ ID NO:33) is canonical class 1, and the Chothia CDR-H2 is canonical class 2. IMGT# IGHV1-69-2*01 (SEQ ID NO:33) belongs to human heavy chain subgroup 1. An example of an acceptor sequence for the 9F5 light chain is the human mature light chain variable region 1911357B-VL_huFrwk (SEQ ID NO:36). An example of an acceptor sequence for the 9F5 light chain is the human mature light chain variable region CAB51297-VL_huFrwk (SEQ ID NO:35). The variable light domain of 9F5 and CAB51297 & 1911357B antibodies also share identical lengths for the CDR-L1, L2 and L3 loops. An example of an acceptor sequence for the 9F5 light chain is the human mature light chain variable region with IGKV2-28*01 & IGKJ2*01 (SEQ ID NO:37). Chothia CDR-L1 of IGKV2-28*01 & IGKJ2*01 (SEQ ID NO:37) is canonical class 4. Chothia CDR-L2 of IGKV2-28*01 & IGKJ2*01 (SEQ ID NO:37) is canonical class 1. Chothia CDR-L3 of IGKV2-28*01 & IGKJ2*01 (SEQ ID NO:37) is canonical class 1. IGKV2-28*01 & IGKJ2*01 (SEQ ID NO:37) belongs to human kappa subgroup 2.

An example of an acceptor sequence for the 10C12 heavy chain is the human mature CAC20421 (SEQ ID NO:218). The variable domains of 10C12 and CAC20421 VH also share identical lengths for the CDR-H1, H2 loops. An example of an acceptor sequence for the 10C12 heavy chain is the human mature heavy chain variable region IMGT# IGHV1-69-2*01 (SEQ ID NO:33). The Chothia CDR-H1 of IMGT# IGHV1-69-2*01 (SEQ ID NO:33) is canonical class 1, and the Chothia CDR-H2 is canonical class 2. IMGT# IGHV1-69-2*01 (SEQ ID NO:33) belongs to human heavy chain subgroup 1. An example of an acceptor sequence for the 10C12 light chain is the human mature light chain variable region CAB51297-VL_huFrwk (SEQ ID NO:35). The variable light domain of 10C12 and CAB51297 VL also share identical lengths for the CDR-L1, L2 and L3 loops. An example of an acceptor sequence for the 10C12 light chain is the human mature light chain variable region with IGKV2-28*01 & IGKJ2*01 (SEQ ID NO:37). Chothia CDR-L1 of IGKV2-28*01 & IGKJ2*01 (SEQ ID NO:37) is canonical class 4. Chothia CDR-L2 of IGKV2-28*01 & IGKJ2*01 (SEQ ID NO:37) is canonical class 1. Chothia CDR-L3 of IGKV2-28*01 & IGKJ2*01 (SEQ ID NO:37) is canonical class 1. IGKV2-28*01 & IGKJ2*01 (SEQ ID NO:37) belongs to human kappa subgroup 2.

An example of an acceptor sequence for the 12C4 heavy chain is the human mature heavy chain variable region of CAC20421-VH_huFrwk (SEQ ID NO:218). The variable domains of 12C4 and CAC20421 VH also share identical lengths for the CDR-H1, H2 loops. An example of an acceptor sequence for the 12C4 heavy chain is the human mature heavy chain variable region IMGT# IGHV1-69-2*01 (SEQ ID NO:33). The Chothia CDR-H1 of IMGT# IGHV1-69-2*01 (SEQ ID NO:33) is canonical class 1, and the Chothia CDR-H2 is canonical class 2. IMGT# IGHV1-69-2*01 (SEQ ID NO:33) belongs to human heavy chain subgroup 1. An example of an acceptor sequence for the 12C4 light chain is the human mature light chain variable region CAB51297-VL_huFrwk (SEQ ID NO:35). The variable light domain of 12C4 and CAB51297 VL also share identical lengths for the CDR-L1, L2 and L3 loops. An example of an acceptor sequence for the 12C4 light chain is the human mature light chain variable region with IGKV2-28*01 & IGKJ2*01 (SEQ ID NO:37). Chothia CDR-L1 of IGKV2-28*01 & IGKJ2*01 (SEQ ID NO:37) is canonical class 4. Chothia CDR-L2 of IGKV2-28*01 & IGKJ2*01 (SEQ ID NO:37) is canonical class 1. Chothia CDR-L3 of IGKV2-28*01 & IGKJ2*01 (SEQ ID NO:37) is canonical class 1. IGKV2-28*01 & IGKJ2*01 (SEQ ID NO:37) belongs to human kappa subgroup 2.

An example of an acceptor sequence for the 17C12 heavy chain is the human mature heavy chain variable region of CAC20421-VH_huFrwk (SEQ ID NO:218). The variable heavy domains of 17C12 and CAC20421also share identical lengths for the CDR-H1, H2 loops. An example of an acceptor sequence for the 17C12 heavy chain is the human mature heavy chain variable region IMGT# IGHV1-69-2*01 (SEQ ID NO:33). The Chothia CDR-H1 of IMGT# IGHV1-69-2*01 (SEQ ID NO:33) is canonical class 1, and the Chothia CDR-H2 is canonical class 2. IMGT# IGHV1-69-2*01 (SEQ ID NO:33) belongs to human heavy chain subgroup 1. An example of an acceptor sequence for the 17C12 light chain is the human mature light chain variable region QD016713-VL_huFrwk (SEQ ID NO:238). The variable light domain of 17C12 and QD016713 antibody also share identical lengths for the CDR-L1, L2 and L3 loops. An example of an acceptor sequence for the 17C12 light chain is the human mature light chain variable region with IGKV2-29*02 & IGKJ4*01 (SEQ ID NO:239).

An example of an acceptor sequence for the 14H3 heavy chain is the human mature heavy chain variable region of QDJ57937-VH_huFrwk (SEQ ID NO:253). The variable domains of 14H3 and QDJ57937 VH also share identical lengths for the CDR-H1, H2 loops. An example of an acceptor sequence for the 14H3 heavy chain is the human mature heavy chain variable region IGHV1-70*04 & IGHJ4*01 (SEQ ID NO:254). An example of an acceptor sequence for the 14H3 light chain is the human mature light chain variable region ABC66914-VL_huFrwk (SEQ ID NO:256). The variable light domain of 14H3 and ABC66914 VL also share identical lengths for the CDR-L1, L2 and L3 loops. An example of an acceptor sequence for the 14H3 light chain is the human mature light chain variable region with IGKV2-28*01 & IGKJ2*01 (SEQ ID NO:37). Chothia CDR-L1 of IGKV2-28*01 & IGKJ2*01 (SEQ ID NO:37) is canonical class 4. Chothia CDR-L2 of IGKV2-28*01 & IGKJ2*01 (SEQ ID NO:37) is canonical class 1. Chothia CDR-L3 of IGKV2-28*01 & IGKJ2*01 (SEQ ID NO:37) is canonical class 1. IGKV2-28*01 & IGKJ2*01 (SEQ ID NO:37) belongs to human kappa subgroup 2.

If more than one human acceptor antibody sequence is selected, a composite or hybrid of those acceptors can be used, and the amino acids used at different positions in the humanized light chain and heavy chain variable regions can be taken from any of the human acceptor antibody sequences used. For example, the human mature heavy chain variable regions of AAN16432-VH_huFrwk (SEQ ID NO:31) and humanized 48G7 Fab with PDB accession code 2RCS-VH_huFrwk (SEQ ID NO:32) were used as hybrid acceptor sequences for the humanization of the 9F5 mature heavy chain variable region. Examples of positions in which these two acceptors differ are position H1 (E or Q), H5 (V or Q), H11 (V or L), H12 (K or V), H20 (V or L), H23 (K or T), H28 (T or N), H38(R or K), H40 (A or R), H42 (G or E), H43 (K or Q), H48 (M or I), H54 (D or N), H66 (R or K), H69 (M or I), H75 (T or S), H76 (D or N), H80 (M or L), H81 (E or Q), H83 (R or T), H108 (L or T), or H109 (V or L). Humanized versions of the 9F5 heavy chain variable region can include either amino acid at any of these positions. Human germline sequence IMGT# IGHV1-69-2*01 (SEQ ID NO:25) was also used as acceptor sequence for humanization of the 9F5 mature heavy chain variable region. For example, the human mature light chain variable regions of CAB51297-VL_huFrwk (SEQ ID NO:35) and 1911357B-VL_huFrwk (SEQ ID NO:36) were used as hybrid acceptor sequences for the humanization of the 9F5 mature light chain variable region. Examples of positions in which these two acceptors differ are positions L7 (S or A), L8 (P or A), L9 (L or F), L11 (L or N), L15 (P or L), L17 (E or T), L18 (P or S), L30 (Y or I), L31 (N or T), L54 (R or L), L60 (D or N), L66 (G or E), or L74 (K or R)). Humanized versions of the9F5 light chain variable region can include either amino acid at any of these positions. Human germline sequence IGKV2-28*01 & IGKJ2*01 (SEQ ID NO:37) was also used as an acceptor sequence for humanization of the 9F5 mature light chain variable region.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly;
(2) is adjacent to a CDR region or within a CDR as defined by Chothia but not Kabat;
(3) otherwise interacts with a CDR region (e.g., is within about 6 A of a CDR region), (e.g., identified by modeling the light or heavy chain on the solved structure of a homologous known immunoglobulin chain); or
(4) is a residue participating in the VL-VH interface.

In an embodiment, humanized sequences are generated using a two-stage PCR protocol that allows introduction of multiple mutations, deletions, and insertions using QuikChange site-directed mutagenesis [Wang, W. and Malcolm, B. A. (1999) BioTechniques 26:680-682)].

Framework residues from classes (1) through (3) as defined by Queen, U.S. Pat No. 5,530,101, are sometimes alternately referred to as canonical and vernier residues. Framework residues that help define the conformation of a CDR loop are sometimes referred to as canonical residues (Chothia & Lesk, J. Mol. Biol. 196:901-917 (1987); Thornton & Martin, J. Mol. Biol. 263:800-815 (1996)). Framework residues that support antigen-binding loop conformations and play a role in fine-tuning the fit of an antibody to antigen are sometimes referred to as vernier residues (Foote & Winter, J. Mol. Biol 224:487-499 (1992)).

Other framework residues that are candidates for substitution are residues creating a potential glycosylation site. Still other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins.

Other framework residues that are candidates for substitution are N-terminal glutamine residues (Q) that may be replaced with glutamic acid (E) to minimize potential for pyroglutamate conversion [Y. Diana Liu, et al., 2011, J. Biol. Chem., 286: 11211-11217]. Glutamic acid (E) conversion to pyroglutamate (pE) occurs more slowly than from glutamine (Q). Because of the loss of a primary amine in the glutamine to pE conversion, antibodies become more acidic. Incomplete conversion produces heterogeneity in the antibody that can be observed as multiple peaks using charge-based analytical methods. Heterogeneity differences may indicate a lack of process control. Exemplary 9F5 humanized heavy chain variable regions with N-terminal glutamine to glutamate substitutions are SEQ ID NO:16 (hu9F5VHv2), SEQ ID NO:17 (hu9F5VHv3), SEQ ID NO:18 (hu9F5VHv4), SEQ ID NO:19 (hu9F5VHv5), SEQ ID NO:20 (hu9F5VHv6), SEQ ID NO:21 (hu9F5VHv7), SEQ ID NO:22 (hu9F5VHv8), SEQ ID NO:109 (hu9F5VHv4_L80P), SEQ ID NO:110 (hu9F5VHv4_L80D), SEQ ID NO:111 (hu9F5VHv4_L82cG), SEQ ID NO:112 (hu9F5VHv4_L82cD), SEQ ID NO:113 (hu9F5VHv4_L82P), SEQ ID NO:114 (hu9F5VHv4_L80G), SEQ ID NO:115 (hu9F5VHv4_L82K), SEQ ID NO:116 (hu9F5VHv4_L82R), SEQ ID NO:117 (hu9F5VHv4_L82E), SEQ ID NO:118 (hu9F5VHv4_L82N), SEQ ID NO:119 (hu9F5VHv4_Y79D), SEQ ID NO:120 (hu9F5VHv4_Y79N), SEQ ID NO:121 (hu9F5VHv4_Y79G), SEQ ID NO:122 (hu9F5VHv5_M80E), SEQ ID NO:123 (hu9F5VHv5_M80G), SEQ ID NO:124 (hu9F5VHv4_L82cS), SEQ ID NO:125 (hu9F5VHv4_Y79Q), SEQ ID NO:126 (hu9F5VHv4 S82aG), SEQ ID NO:127 (hu9F5VHv9), SEQ ID NO:128 (hu9F5VHv10), SEQ ID NO:129 (hu9F5VHv10_L82cG). An exemplary 10C12 humanized heavy chain variable region with N-terminal glutamine to glutamate substitution is SEQ ID NO:215 (hu10C12VHv2). An exemplary 12C4 humanized heavy chain variable region with N-terminal glutamine to glutamate substitution is SEQ ID NO:222 (hu12C4VHv2). An exemplary 17C12 humanized heavy chain variable region with N-terminal glutamine to glutamate substitution is SEQ ID NO:233 (hu17C12VHv2).

Exemplary humanized antibodies include humanized forms of the mouse 9F5, designated Hu9F5.

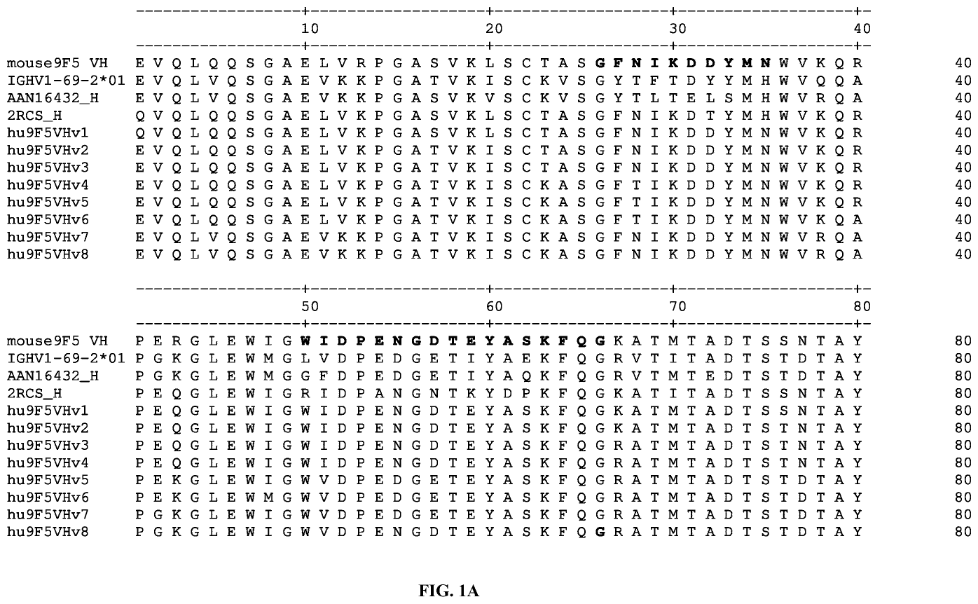

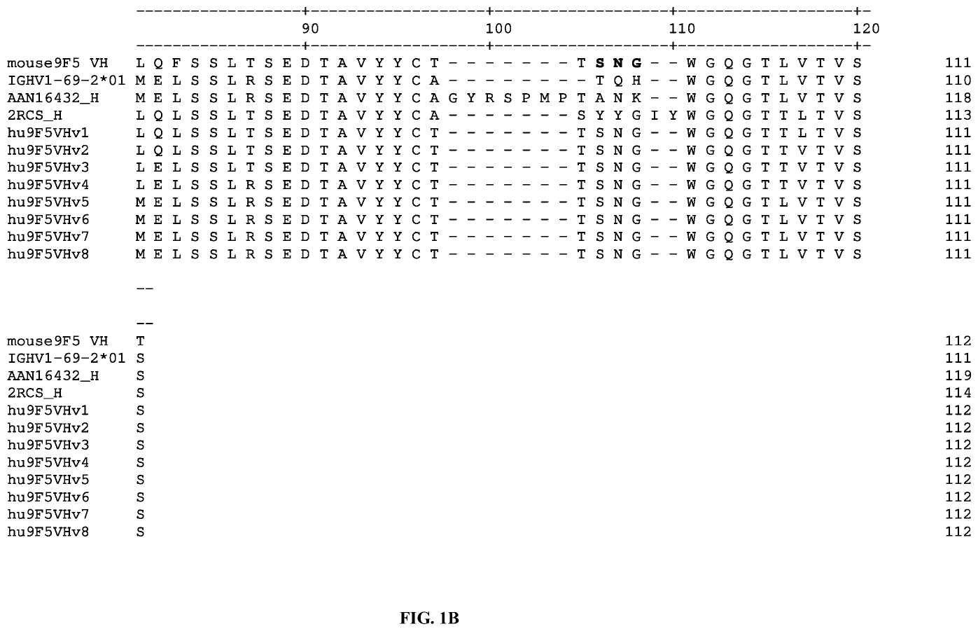

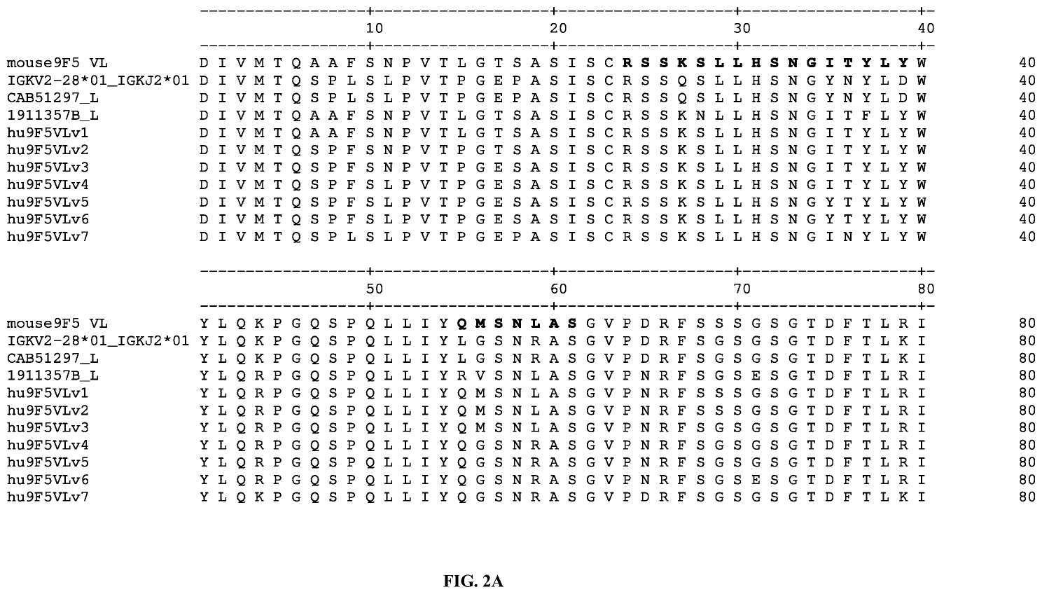

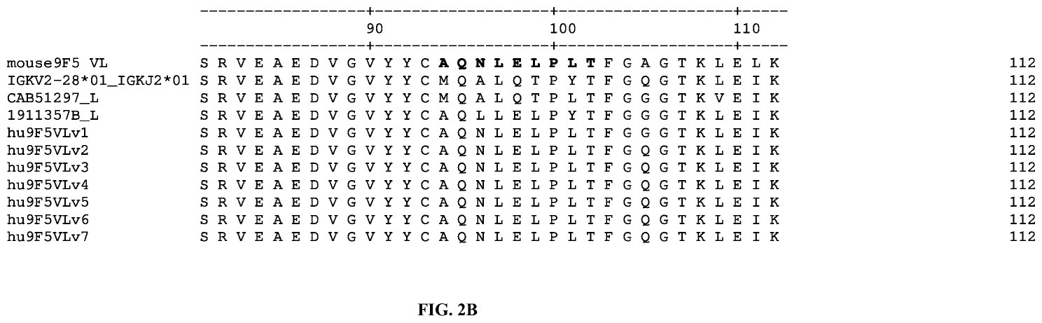

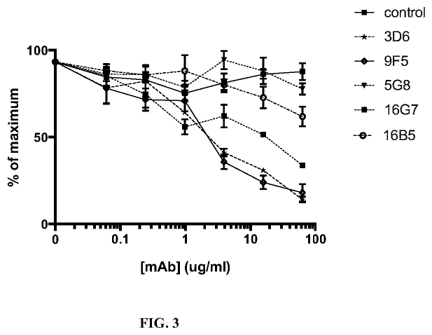

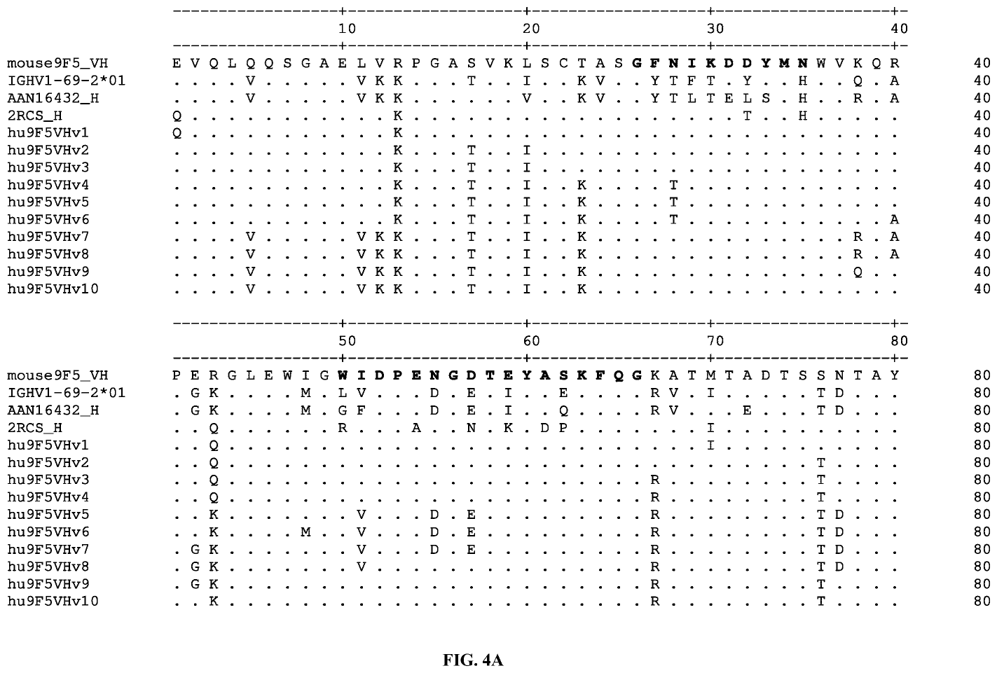

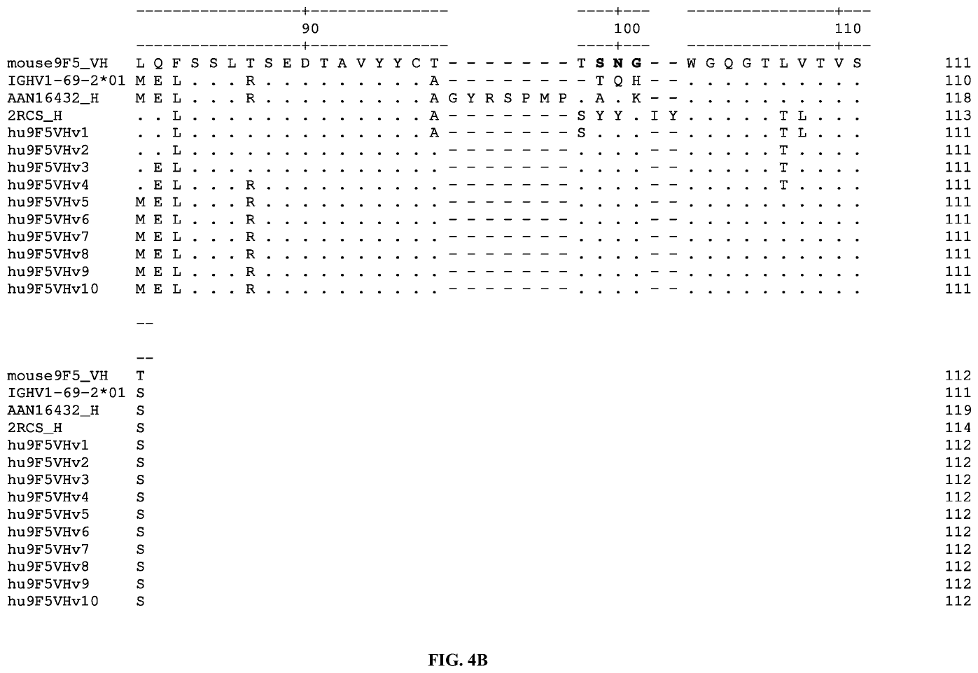

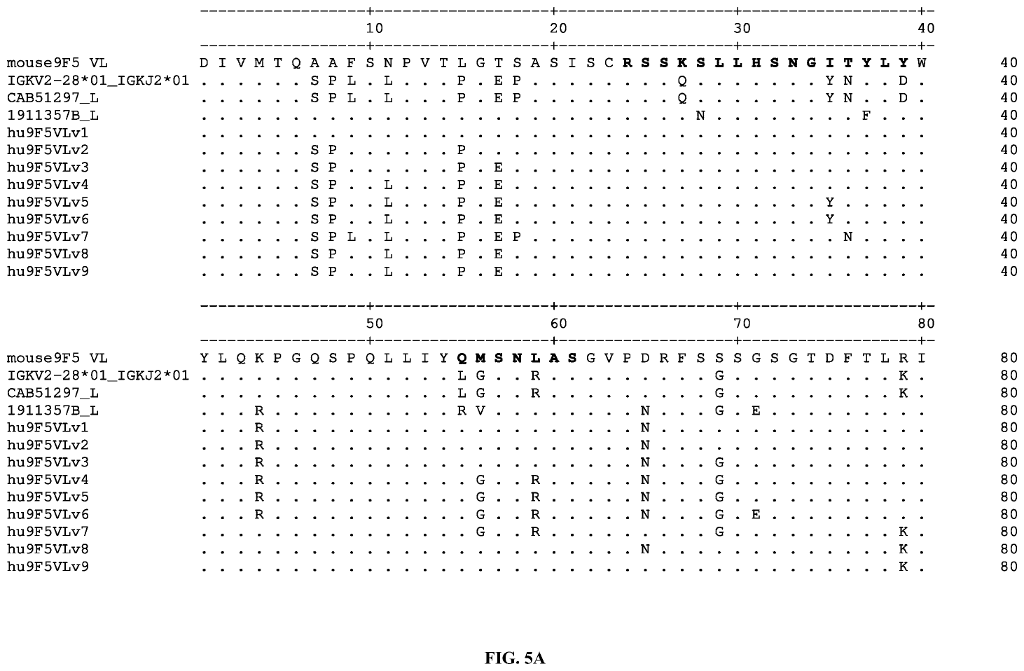

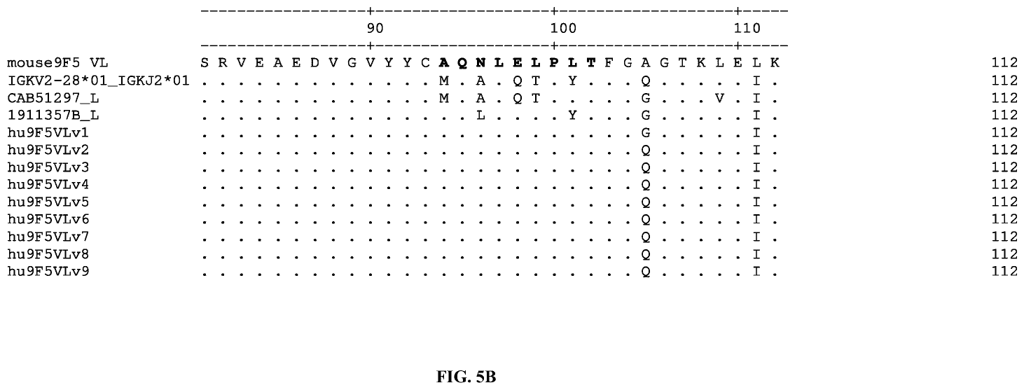

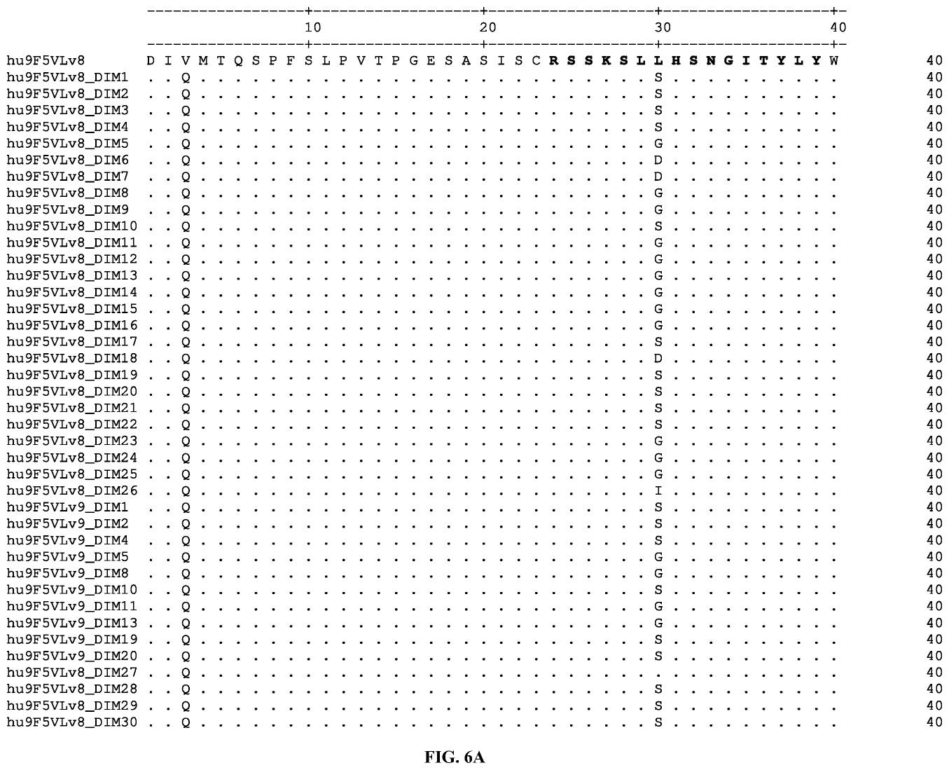

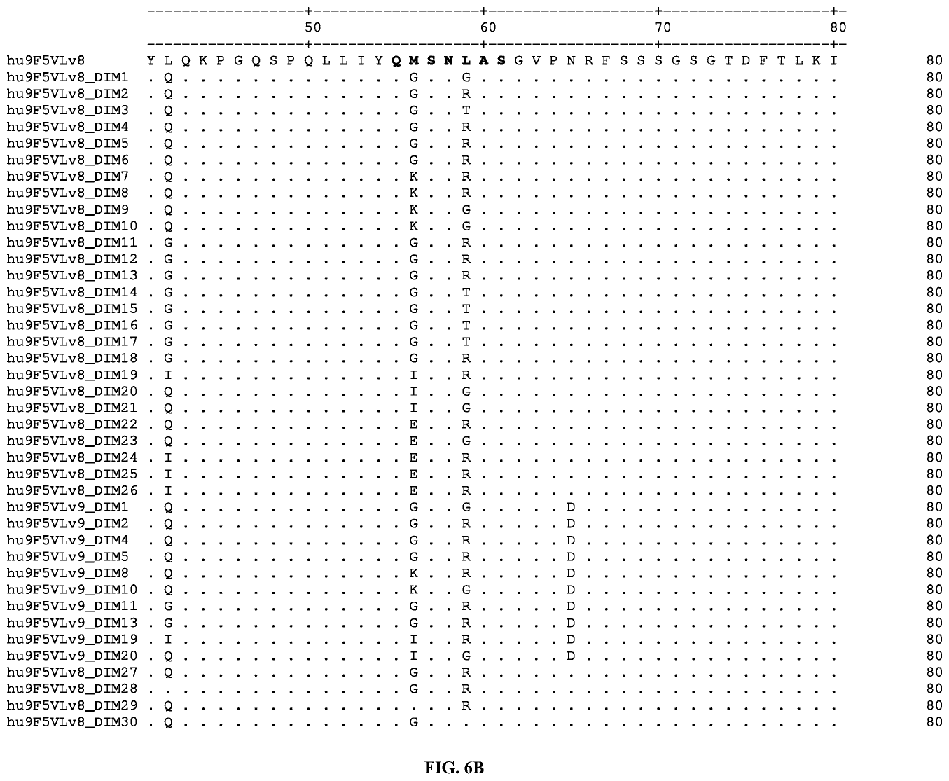

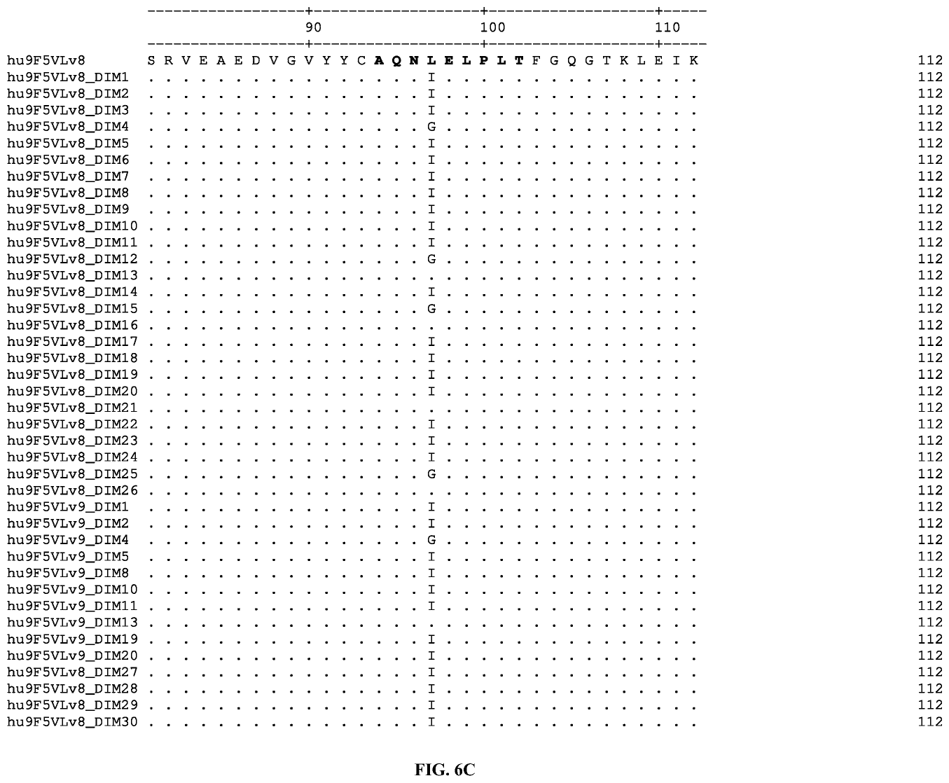

The mouse antibody 9F5 comprises mature heavy and light chain variable regions having amino acid sequences comprising SEQ ID NO:7 and SEQ ID NO:11, respectively. The invention provides 29 exemplified humanized mature heavy chain variable regions: hu9F5VHv1 (SEQ ID NO:15), hu9F5VHv2 (SEQ ID NO:16), hu9F5VHv3 (SEQ ID NO:17), hu9F5VHv4 (SEQ ID NO:18), hu9F5VHv5 (SEQ ID NO:19), hu9F5VHv6 (SEQ ID NO:20 hu9F5VHv7 (SEQ ID NO:21), hu9F5VHv8 (SEQ ID NO:22), hu9F5VHv4_L80P (SEQ ID NO:109), hu9F5VHv4_L80D (SEQ ID NO:110), hu9F5VHv4_L82cG (SEQ ID NO:111), hu9F5VHv4_L82cD (SEQ ID NO:112), hu9F5VHv4_L82P (SEQ ID NO:113), hu9F5VHv4_L80G (SEQ ID NO:114), hu9F5VHv4_L82K (SEQ ID NO 115:), hu9F5VHv4_L82R (SEQ ID NO:116), hu9F5VHv4_L82E (SEQ ID NO:117), hu9F5VHv4_L82N (SEQ ID NO:118), hu9F5VHv4_Y79D (SEQ ID NO:119), hu9F5VHv4_Y79N (SEQ ID NO:120), hu9F5VHv4_Y79G (SEQ ID NO:121), hu9F5VHv5_M80E (SEQ ID NO:122), hu9F5VHv5_M80G (SEQ ID NO:123), hu9F5VHv4_L82cS (SEQ ID NO:124), hu9F5VHv4_Y79Q (SEQ ID NO:125), hu9F5VHv4_S82aG (SEQ ID NO:126), hu9F5VHv9 (SEQ ID NO:127), hu9F5VHv10 (SEQ ID NO:128), and hu9F5VHv10 L82cG (SEQ ID NO:129). The invention further provides 95 exemplified mature light chain variable regions hu9F5VLv1 (SEQ ID NO:23), hu9F5VLv2 (SEQ ID NO:24), hu9F5VLv3 (SEQ ID NO:25), hu9F5VLv4 (SEQ ID NO:26), hu9F5VLv5 (SEQ ID NO:27), hu9F5VLv6 (SEQ ID NO 28:), hu9F5VLv7 (SEQ ID NO:29), hu9F5VLv8 (SEQ ID NO:130), hu9F5VLv9 (SEQ ID NO:131), hu9F5VLv2_M51E (SEQ ID NO:61), hu9F5VLv2_M51D (SEQ ID NO:62), hu9F5VLv2_L27cD (SEQ ID NO:63), hu9F5VLv2_L27cG (SEQ ID NO:64), hu9F5VLv2_L27cS (SEQ ID NO:65), hu9F5VLv2_L27cE (SEQ ID NO:66), hu9F5VLv2_I30E (SEQ ID NO 67:), hu9F5VLv2_I30K (SEQ ID NO:68), hu9F5VLv2_L27cT (SEQ ID NO:69), hu9F5VLv2_L27cN (SEQ ID NO:70, hu9F5VLv2_L27bD (SEQ ID NO:71), hu9F5VLv2_I30G (SEQ ID NO:72), hu9F5VLv2_L33N (SEQ ID NO:73), hu9F5VLv2_L27cA (SEQ ID NO:74), hu9F5VLv2_L33T (SEQ ID NO:75), hu9F5VLv2_L33S (SEQ ID NO:76), hu9F5VLv2_L33R (SEQ ID NO:77), hu9F5VLv2_I30Q (SEQ ID NO:78), hu9F5VLv2_L27bT (SEQ ID NO:79, hu9F5VLv2_T31G (SEQ ID NO:80), hu9F5VLv2_L27bQ (SEQ ID NO:81), hu9F5VLv2_L33G (SEQ ID NO:82), hu9F5VLv2_L27cP (SEQ ID NO:83), hu9F5VLv2_V78R (SEQ ID NO:84), hu9F5VLv2_I75D (SEQ ID NO:85), hu9F5VLv2_V78D (SEQ ID NO:86), hu9F5VLv2_V78E (SEQ ID NO:87), hu9F5VLv2_V78P (SEQ ID NO:88), hu9F5VLv2_V78K (SEQ ID NO:89), hu9F5VLv2_R77D (SEQ ID NO:90), hu9F5VLv2_V78G (SEQ ID NO:91), hu9F5VLv2_S76P (SEQ ID NO:92), hu9F5VLv2_I75P (SEQ ID NO:93), hu9F5VLv2_I75Q (SEQ ID NO:94), hu9F5VLv2_I75G (SEQ ID NO:95), hu9F5VLv2_L73P (SEQ ID NO:96), hu9F5VLv2_L73G (SEQ ID NO:97), hu9F5VLv2_V78Q (SEQ ID NO:98), hu9F5VLv2_S76G (SEQ ID NO:99), hu9F5VLv2_L92D (SEQ ID NO:100), hu9F5VLv2_Y86T (SEQ ID NO:101), hu9F5VLv2_L92E (SEQ ID NO:102), hu9F5VLv2_L92G (SEQ ID NO:103), hu9F5VLv2_L92Q (SEQ ID NO:104), hu9F5VLv2_L93G (SEQ ID NO:105), hu9F5VLv2_V85G (SEQ ID NO:106), hu9F5VLv2_L92T (SEQ ID NO:107), hu9F5VLv2_A89G (SEQ ID NO:108), hu9F5VLv8_DIM1 (SEQ ID NO:132), hu9F5VLv8_DIM2 (SEQ ID NO:133), hu9F5VLv8_DIM3 (SEQ ID NO:134), hu9F5VLv8_DIM4 (SEQ ID NO:135), hu9F5VLv8_DIM5 (SEQ ID NO:136), hu9F5VLv8_DIM6 (SEQ ID NO:137), hu9F5VLv8_DIM7 (SEQ ID NO:138), hu9F5VLv8_DIM8 (SEQ ID NO:139), hu9F5VLv8_DIM9 (SEQ ID NO:140), hu9F5VLv8_DIM10 (SEQ ID NO:141), hu9F5VLv8_DIM11 (SEQ ID NO:142), hu9F5VLv8_DIM12 (SEQ ID NO:143), hu9F5VLv8_DIM13 (SEQ ID NO:144), hu9F5VLv8_DIM14 (SEQ ID NO:145), hu9F5VLv8_DIM15 (SEQ ID NO:146), hu9F5VLv8_DIM16 (SEQ ID NO:147), hu9F5VLv8_DIM17 (SEQ ID NO:148), hu9F5VLv8_DIM18 (SEQ ID NO:149), hu9F5VLv8_DIM 19 (SEQ ID NO:150), hu9F5VLv8_DIM20 (SEQ ID NO:151), hu9F5VLv8_DIM21 (SEQ ID NO:152), hu9F5VLv8_DIM22 (SEQ ID NO:153), hu9F5VLv8_DIM23 (SEQ ID NO:154), hu9F5VLv8_DIM24 (SEQ ID NO:155), hu9F5VLv8_DIM25 (SEQ ID NO:156), hu9F5VLv8_DIM26 (SEQ ID NO:157), hu9F5VLv8_DIM27 (SEQ ID NO:158), hu9F5VLv8_DIM28 (SEQ ID NO:159), hu9F5VLv8_DIM29 (SEQ ID NO:160), hu9F5VLv8_DIM30 (SEQ ID NO:161), hu9F5VLv9_DIM1 (SEQ ID NO:162), hu9F5VLv9_DIM2 (SEQ ID NO:163), hu9F5VLv9_DIM4 (SEQ ID NO:164), hu9F5VLv9_DIM5 (SEQ ID NO:165), hu9F5VLv9_DIM8) (SEQ ID NO:166), hu9F5VLv9_DIM10 (SEQ ID NO:167), hu9F5VLv9_DIM11 (SEQ ID NO:168), (hu9F5VLv9_DIM13 (SEQ ID NO:169), hu9F5VLv9_DIM19 (SEQ ID NO:170), and hu9F5VLv9_DIM20 (SEQ ID NO:171). FIGS. 1A-1B and 4A-4B show alignments of the heavy chain variable region of murine 9F5 and various humanized antibodies. FIGS. 2A-2B and 5A-5B, show alignments of the light chain variable region of murine 9F5 and various humanized antibodies. FIG. 6A-6C show alignments of the light chain variable region of humanized variant hu9F5VLv8 and various humanized antibodies,

For reasons such as possible influence on CDR conformation and/or binding to antigen, mediating interaction between heavy and light chains, interaction with the constant region, being a site for desired or undesired post-translational modification, being an unusual residue for its position in a human variable region sequence and therefore potentially immunogenic, reducing aggregation potential, and other reasons, the following 48 variable region framework positions were considered as candidates for substitutions in the 95 exemplified human mature light chain variable regions and the 29 exemplified human mature heavy chain variable regions, as further specified in the examples: L3 (V3Q), L7 (A7S), L8 (A8P), L9 (F9L), L11 (N11L), L15 (L151P), L17 (T17E), L18 (518P), L37 (L37Q, L37G, L37I)), L39 (R39K), L60 (N60D), L64 (G64S), L66 (E66G), L73 (L73P, L73G), L74 (R74K), L75 (I75D, I75P, I75Q, I75G), L76 (S76P, S76G), L77 (R77D), L78 (V78R, V78D, V78E, V78P, V78K, V78G, V78Q), L85 (V85G), L86 (Y86T), L100 (G100Q), H1 (Q1E), H5 (Q5V), H11 (L11V), H12 (V12K), H17 (S17T), H20 (L201), H23 (T23K), H38 (K38R, K38Q), H40 (R40A), H42 (E42G), H43 (Q43K), H48 (I48M), H66 (K66R), H69 (I69M), H75 (575T), H76 (N76D), H79 (Y79Q, Y79D, Y79N, Y79G), H80 (L80M, L80P, L80D, L80G, L80E), H81 (Q81E), H82 (L82P, L82K, L82R, L82R, L82E, L82N), H82a (S82aG), H82c (L82cG, L82cD, L82cS), H83 (T83R), H93 (A93T), H94 (S94T), H108 (T108L), and H109 (L109V). The following 14 variable region CDR positions were considered as candidates for substitutions in the 95 exemplified human mature light chain variable regions and 29 exemplified human mature heavy chain variable regions, as further specified in the examples: L27b (L27bD, L27bT, L27bQ), L27c (L27cD, L27cG, L27cS, L27cE, L27cT, L27cN, L27cA, L27cP, L27cI), L30 (I30Y, 130E, 130K, 130G, I30Q), L31 (T31N, T31G), L33 (L33N, L33T, L33S, L33R, L33G), L51 (M51G, M51E, M51D, M51K, M51I), L54 (L54R, L54G, L54T), L89 (A89G), L92 (L92D, L92E, L92G, L92Q, L92T, L92I), L93 (E93G), H28 (N28T), H51 (I48M), H54 (N54D), and H56 (D56E). In some humanized 9F5 antibodies, Kabat-Chothia Composite CDR-H1 has an amino acid sequence comprising SEQ ID NO:50. In some humanized 9F5 antibodies, Kabat-Chothia Composite CDR-H1 has an amino acid sequence comprising SEQ ID NO:50, and Kabat CDR-H2 has an amino acid sequence comprising SEQ ID NO:51. In some humanized 9F5 antibodies, Kabat CDR-H2 has an amino acid sequence comprising SEQ ID NO:51. In some humanized 9F5 antibodies, Kabat CDR-H2 has an amino acid sequence comprising SEQ ID NO:52. In some humanized 9F5 antibodies, Kabat CDR-L2 has an amino acid sequence comprising SEQ ID NO:55. In some humanized 9F5 antibodies, Kabat CDR-L1 has an amino acid sequence comprising SEQ ID NO:53 and Kabat CDR-L2 has an amino acid sequence comprising SEQ ID NO:55. In some humanized 9F5 antibodies, Kabat CDR-L1 has an amino acid sequence comprising SEQ ID NO:54, and Kabat CDR-L2 has an amino acid sequence comprising SEQ ID NO:55. In some humanized 9F5 antibodies, Kabat CDR-L1 has an amino acid sequence comprising a sequence selected from the group consisting of SEQ ID NOs:172-193. In some humanized 9F5 antibodies, Kabat CDR-L2 has an amino acid sequence comprising sequence selected from the group consisting of SEQ ID NOs:194-205. In some humanized 9F5 antibodies, Kabat CDR-L3 has an amino acid sequence comprising a sequence selected from the group consisting of SEQ ID NOs:206-213.

Here, as elsewhere, the first-mentioned residue is the residue of a humanized antibody formed by grafting Kabat CDRs or a composite Chothia-Kabat CDR in the case of CDR-H1 into a human acceptor framework, and the second-mentioned residue is a residue being considered for replacing such residue. Thus, within variable region frameworks, the first mentioned residue is human, and within CDRs, the first mentioned residue is mouse.

Exemplified antibodies include any permutations or combinations of the exemplified mature heavy and light chain variable regions hu9F5VHv1/hu9F5VLv1, hu9F5VHv1/hu9F5VLv2, hu9F5VHv1/hu9F5VLv3, hu9F5VHv1/hu9F5VLv4, hu9F5VLv1/hu9F5VLv5, hu9F5VHv1/hu9F5VLv6, hu9F5VHv1/hu9F5VLv7, hu9F5VHv2/hu9F5VLv1, hu9F5VHv2/hu9F5VLv2, hu9F5VHv2/hu9F5VLv3, hu9F5VHv2/hu9F5VLv4, hu9F5VLv2/hu9F5VLv5, hu9F5VHv2/hu9F5VLv6, hu9F5VHv2/hu9F5VLv7, hu9F5VHv3/hu9F5VLv1, hu9F5VHv3/hu9F5VLv2, hu9F5VHv3/hu9F5VLv3, hu9F5VHv3/hu9F5VLv4, hu9F5VLv3/hu9F5VLv5, hu9F5VHv3/hu9F5VLv6, hu9F5VHv3/hu9F5VLv7, hu9F5VHv4/hu9F5VLv1, hu9F5VHv4/hu9F5VLv2, hu9F5VHv4/hu9F5VLv3, hu9F5VHv4/hu9F5VLv4, hu9F5VLv4/hu9F5VLv5, hu9F5VHv4/hu9F5VLv6, hu9F5VHv4/hu9F5VLv7, hu9F5VHv5/hu9F5VLv1, hu9F5VHv5/hu9F5VLv2, hu9F5VHv5/hu9F5VLv3, hu9F5VHv5/hu9F5VLv4, hu9F5VLv5/hu9F5VLv5, hu9F5VHv5/hu9F5VLv6, hu9F5VHv5/hu9F5VLv7, hu9F5VHv6/hu9F5VLv1, hu9F5VHv6/hu9F5VLv2, hu9F5VHv6/hu9F5VLv3, hu9F5VHv6/hu9F5VLv4, hu9F5VHv6/hu9F5VLv5, hu9F5VHv6/hu9F5VLv6, hu9F5VHv6/hu9F5VLv7, hu9F5VHv7/hu9F5VLv1, hu9F5VHv7/hu9F5VLv2, hu9F5VHv7/hu9F5VLv3, hu9F5VHv7/hu9F5VLv4, hu9F5VLv7/hu9F5VLv5, hu9F5VHv7/hu9F5VLv6, hu9F5VHv7/hu9F5VLv7, hu9F5VHv8/hu9F5VLv1, hu9F5VHv8/hu9F5VLv2, hu9F5VHv8/hu9F5VLv3, hu9F5VHv8/hu9F5VLv4, hu9F5VLv8/hu9F5VLv5, hu9F5VHv8/hu9F5VLv6, hu9F5VHv8/hu9F5VLv7.

Exemplified antibodies include any permutations or combinations of the exemplified mature heavy chain variable regions hu9F5VHv1 (SEQ ID NO:15), hu9F5VHv2 (SEQ ID NO:16), hu9F5VHv3 (SEQ ID NO:17), hu9F5VHv4 (SEQ ID NO:18), hu9F5VHv5 (SEQ ID NO:19), hu9F5VHv6 (SEQ ID NO:20), hu9F5VHv7 (SEQ ID NO:21), hu9F5VHv8 (SEQ ID NO:22), hu9F5VHv9 (SEQ ID NO:127), hu9F5VHv10 (SEQ ID NO:128), hu9F5VHv10_L82cG (SEQ ID NO:129), hu9F5VHv4_L80P (SEQ ID NO:109), hu9F5VHv4_L80D (SEQ ID NO:110), hu9F5VHv4_L82cG (SEQ ID NO:111), hu9F5VHv4_L82cD (SEQ ID NO:112), hu9F5VHv4_L82P (SEQ ID NO:113), hu9F5VHv4_L80G (SEQ ID NO:114), hu9F5VHv4_L82K (SEQ ID NO:115), hu9F5VHv4_L82R (SEQ ID NO:116), hu9F5VHv4_L82E (SEQ ID NO:117), hu9F5VHv4_L82N (SEQ ID NO:118), hu9F5VHv4_Y79D (SEQ ID NO:119), hu9F5VHv4_Y79N (SEQ ID NO:120), hu9F5VHv4_Y79G (SEQ ID NO:121), hu9F5VHv5_M80E (SEQ ID NO:122), hu9F5VHv5_M80G (SEQ ID NO:123), hu9F5VHv4_L82Cs (SEQ ID NO:124), hu9F5VHv4_Y79Q (SEQ ID NO:125), and hu9F5VHv4 S82aG (SEQ ID NO:126), with any of the exemplified mature light chain variable regions hu9F5VLv1 (SEQ ID NO:23), hu9F5VLv2_ (SEQ ID NO:24), hu9F5VLv3 (SEQ ID NO:25), hu9F5VLv4 (SEQ ID NO:26), hu9F5VLv5 (SEQ ID NO:27, hu9F5VLv6 (SEQ ID NO:28), hu9F5VLv7 (SEQ ID NO:29), hu9F5VLv8 (SEQ ID NO:130), hu9F5VLv9 (SEQ ID NO:131), hu9F5VLv2_M51E (SEQ ID NO:61), hu9F5VLv2_M51D (SEQ ID NO:62), hu9F5VLv2_L27cD (SEQ ID NO:63), hu9F5VLv2_L27cG (SEQ ID NO:64), hu9F5VLv2_L27cS (SEQ ID NO:65), hu9F5VLv2_L27cE (SEQ ID NO:66), hu9F5VLv2_I30E (SEQ ID NO:67), hu9F5VLv2_I30K (SEQ ID NO:68), hu9F5VLv2_L27cT (SEQ ID NO:69), hu9F5VLv2_L27cN (SEQ ID NO:70), hu9F5VLv2_L27bD (SEQ ID NO:71), hu9F5VLv2_I30G, (SEQ ID NO:72), hu9F5VLv2_L33N (SEQ ID NO:73), hu9F5VLv2_L27cA (SEQ ID NO:74), hu9F5VLv2_L33T (SEQ ID NO:75), hu9F5VLv2_L33S (SEQ ID NO:76), hu9F5VLv2_L33R (SEQ ID NO:77), hu9F5VLv2_I30Q (SEQ ID NO:78), hu9F5VLv2_L27bT (SEQ ID NO:79), hu9F5VLv2_T31G (SEQ ID NO:80), hu9F5VLv2_L27bQ (SEQ ID NO:81), hu9F5VLv2_L33G (SEQ ID NO:82), hu9F5VLv2_L27cP (SEQ ID NO:83), hu9F5VLv2_V78R (SEQ ID NO:84), hu9F5VLv2_I75D (SEQ ID NO:85), hu9F5VLv2_V78D (SEQ ID NO:86), hu9F5VLv2_V78E (SEQ ID NO:87), hu9F5VLv2_V78P (SEQ ID NO:88), hu9F5VLv2_V78K (SEQ ID NO:89), hu9F5VLv2_R77D (SEQ ID NO:90), hu9F5VLv2_V78G (SEQ ID NO:91), hu9F5VLv2_S76P (SEQ ID NO:92), hu9F5VLv2_I75P (SEQ ID NO:93), hu9F5VLv2_I75Q (SEQ ID NO:94), hu9F5VLv2_I75G (SEQ ID NO:95), hu9F5VLv2_L73P (SEQ ID NO:96), hu9F5VLv2_L73G (SEQ ID NO:97), hu9F5VLv2_V78Q (SEQ ID NO:98), hu9F5VLv2_S76G (SEQ ID NO:99), hu9F5VLv2_L92D (SEQ ID NO:100), hu9F5VLv2_Y86T (SEQ ID NO:101), hu9F5VLv2_L92E (SEQ ID NO:102), hu9F5VLv2_L92G (SEQ ID NO:103), hu9F5VLv2_L92Q (SEQ ID NO:104), hu9F5VLv2_L93G (SEQ ID NO:105), hu9F5VLv2_V85G (SEQ ID NO:106), hu9F5VLv2_L92T (SEQ ID NO:107), hu9F5VLv2_A89G (SEQ ID NO:108), hu9F5VLv8_DIM1 (SEQ ID NO:132), hu9F5VLv8_DIM2 (SEQ ID NO:133), hu9F5VLv8_DIM3 (SEQ ID NO:134), hu9F5VLv8_DIM4 (SEQ ID NO:135), hu9F5VLv8_DIM5 (SEQ ID NO:136), hu9F5VLv8_DIM6 (SEQ ID NO:137), hu9F5VLv8_DIM7 (SEQ ID NO:138), hu9F5VLv8_DIM5 (SEQ ID NO:139), hu9F5VLv8_DIM9 (SEQ ID NO:140), hu9F5VLv8_DIM10 (SEQ ID NO:141), hu9F5VLv8_DIM11 (SEQ ID NO:142), hu9F5VLv8_DIM12 (SEQ ID NO:143), hu9F5VLv8_DIM13 (SEQ ID NO:144), hu9F5VLv8_DIM14 (SEQ ID NO:145), hu9F5VLv8_DIM15 (SEQ ID NO:146), hu9F5VLv8_DIM16 (SEQ ID NO:147), hu9F5VLv8_DIM17 (SEQ ID NO:148), hu9F5VLv8_DIM18 (SEQ ID NO:149), hu9F5VLv8_DIM19 (SEQ ID NO:150), hu9F5VLv8_DIM20 (SEQ ID NO:151), hu9F5VLv8_DIM21 (SEQ ID NO:152), hu9F5VLv8_DIM22 (SEQ ID NO:153), hu9F5VLv8_DIM23 (SEQ ID NO:154), hu9F5VLv8_DIM24 (SEQ ID NO:155), hu9F5VLv8_DIM25 (SEQ ID NO:156), hu9F5VLv8_DIM26 (SEQ ID NO:157), hu9F5VLv8_DIM27 (SEQ ID NO:158), hu9F5 VLv8_DIM28 (SEQ ID NO:159), hu9F5VLv8_DIM29 (SEQ ID NO:160), hu9F5VLv8_DIM30 (SEQ ID NO:161), hu9F5VLv9_DIM1 (SEQ ID NO:162), hu9F5VLv9_DIM2 (SEQ ID NO:163), hu9F5VLv9_DIM4 (SEQ ID NO:164), hu9F5VLv9_DIM5 (SEQ ID NO:165), hu9F5VLv9_DIM8 (SEQ ID NO:166), hu9F5VLv9_DIM10 (SEQ ID NO:167), hu9F5VLv9_DIM11 (SEQ ID NO:168), hu9F5 VLv9_DIM13 (SEQ ID NO:169), hu9F5VLv9_DIM19 (SEQ ID NO:170), and hu9F5VLv9_DIM20 (SEQ ID NO:171),

The invention provides an antibody in which humanized heavy chain variable region hu9F5VHv9 (SEQ ID NO:127) is combined with humanized light chain variable region hu9F5VLv8_DIM18 (also known as hu9F5VLv8_V3Q, L27cD, L37G, M51G, L54R, L92I, SEQ ID NO:149). The invention provides an antibody in which humanized heavy chain variable region hu9F5VHv9 (SEQ ID NO:127) is combined with humanized light chain variable region hu9F5VLv8_DIM11 (also known as hu9F5VLv8_V3Q, L27cG, L37G, M51G, L54R, L92I, SEQ ID NO:142). The invention provides an antibody in which humanized heavy chain variable region hu9F5VHv9 (SEQ ID NO:127) is combined with humanized light chain variable region hu9F5VLv8_DIM28 (also known as hu9F5VLv8_V3Q, L27cS, M51G, L54R, L92I, SEQ ID NO:159). The invention provides an antibody in which humanized heavy chain variable region hu9F5VHv9 (SEQ ID NO:127) is combined with humanized light chain variable region hu9F5VLv8_DIM17 (also known as hu9F5VLv8_V3Q, L27cS, L37G, M51G, L54T, L92I, SEQ ID NO:148).

The invention provides an antibody in which humanized heavy chain variable region hu9F5VHv9 (SEQ ID NO:127) is combined with humanized light chain variable region /hu9F5VLv8_DIM6 (also known as hu9F5VLv8_V3Q, L27cD, L37Q, M51G, L54R, L92I, SEQ ID NO:137). The invention provides an antibody in which humanized heavy chain variable region hu9F5VHv9 (SEQ ID NO:127) is combined with humanized light chain variable region hu9F5VLv8_DIM14 (also known as hu9F5VLv8_V3Q, L27cG, L37G, M51G, L54T, L92I, SEQ ID NO:145). The invention provides an antibody in which humanized heavy chain variable region hu9F5VHv9 (SEQ ID NO:127) is combined with humanized light chain variable region hu9F5VLv8_DIM5 (also known as hu9F5VLv8_V3Q, L27cG, L37Q, M51G, L54R, L92I, SEQ ID NO:136). The invention provides an antibody in which humanized heavy chain variable region hu9F5VHv9 (SEQ ID NO:127) is combined with humanized light chain variable region hu9F5VLv8_DIM7 (also known as hu9F5VLv8_V3Q, L27cD, L37Q, M51K, L54R, L92I, SEQ ID NO:138).

The invention provides an antibody in which humanized heavy chain variable region hu9F5VHv9 (SEQ ID NO:127) is combined with humanized light chain variable region hu9F5VLv8_DIM27 (also known as hu9F5VLv8_V3Q, L37Q, M51G, L54R, L92I, SEQ ID NO:158). The invention provides an antibody in which humanized heavy chain variable region hu9F5VHv9 (SEQ ID NO:127) is combined with humanized light chain variable region hu9F5VLv8_DIM12 (also known as hu9F5VLv8_V3Q, L27cG, L37G, M51G, L54R, L92G, SEQ ID NO:143). The invention provides an antibody in which humanized heavy chain variable region hu9F5VHv9 (SEQ ID NO:127) is combined with humanized light chain variable region hu9F5VLv8_DIM13 (also known as hu9F5VLv8_V3Q, L27cG, L37G, M51G, L54R, SEQ ID NO:144). The invention provides an antibody in which humanized heavy chain variable region hu9F5VHv9 (SEQ ID NO:127) is combined with humanized light chain variable region hu9F5VLv8_DIM2 (also known as hu9F5VLv8_V3Q, L27cS, L37Q, M51G, L54R, L92I, SEQ ID NO:133).

The invention provides an antibody in which humanized heavy chain variable region hu9F5VHv9 (SEQ ID NO:127) is combined with humanized light chain variable region hu9F5VLv8_DIM29 (also known as hu9F5VLv8_V3Q, L27cS, L37Q, L54R, L92I, SEQ ID NO:160). The invention provides an antibody in which humanized heavy chain variable region hu9F5VHv9 (SEQ ID NO:127) is combined with humanized light chain variable region /hu9F5VLv8_DIM30 (also known as hu9F5VLv8_V3Q, L27cS, L37Q, M51G, L92I, SEQ ID NO:161). The invention provides an antibody in which humanized heavy chain variable region hu9F5VHv9 (SEQ ID NO:127) is combined with humanized light chain variable region hu9F5VLv8_DIM8 (also known as hu9F5VLv8_V3Q, L27cG, L37Q, M51K, L54R, L92I, SEQ ID NO:139).

The invention provides an antibody in which humanized heavy chain variable region hu9F5VHv10 (SEQ ID NO:128) is combined with humanized light chain variable region hu9F5VLv9_DIM11 (also known as hu9F5VLv9_V3Q, L27cG, L37G, M51G, L54R, L92I, SEQ ID NO:168).

The invention provides variants of the 9F5 humanized antibody in which the humanized mature heavy chain variable region shows at least 90%, 95%, 96%, 97%, 98%, or 99% identity to hu9F5VHv1 (SEQ ID NO:15), hu9F5VHv2 (SEQ ID NO:16), hu9F5VHv3 (SEQ ID NO:17), hu9F5VHv4 (SEQ ID NO:18), hu9F5VHv5 (SEQ ID NO:19), hu9F5VHv6 (SEQ ID NO:20), hu9F5VHv7 (SEQ ID NO:21), hu9F5VHv8 (SEQ ID NO:22), hu9F5VHv9 (SEQ ID NO:127), hu9F5VHv10 (SEQ ID NO:128), hu9F5VHv10 L82cG (SEQ ID NO:129), hu9F5VHv4_L80P (SEQ ID NO:109), hu9F5VHv4_L80D (SEQ ID NO:110), hu9F5VHv4_L82cG (SEQ ID NO:111), hu9F5VHv4_L82cD (SEQ ID NO:112), hu9F5VHv4_L82P (SEQ ID NO:113), hu9F5VHv4_L80G (SEQ ID NO:114), hu9F5VHv4_L82K (SEQ ID NO:115), hu9F5VHv4_L82R (SEQ ID NO:116), hu9F5VHv4_L82E (SEQ ID NO:117), hu9F5VHv4_L82N (SEQ ID NO:118), hu9F5VHv4_Y79D (SEQ ID NO:119), hu9F5VHv4_Y79N (SEQ ID NO:120), hu9F5VHv4_Y79G (SEQ ID NO:121), hu9F5VHv5_M80E (SEQ ID NO:122), hu9F5VHv5_M80G (SEQ ID NO:123), hu9F5VHv4_L82Cs (SEQ ID NO:124), hu9F5VHv4_Y79Q (SEQ ID NO:125), or hu9F5VHv4 S82aG (SEQ ID NO:126), and the humanized mature light chain variable region shows at least 90%, 95%, 96%, 97%, 98%, or 99% identity to hu9F5VLv1 (SEQ ID NO:23), hu9F5VLv2_(SEQ ID NO:24), hu9F5VLv3 (SEQ ID NO:25), hu9F5VLv4 (SEQ ID NO:26), hu9F5VLv5 (SEQ ID NO:27, hu9F5VLv6 (SEQ ID NO:28), hu9F5VLv7 (SEQ ID NO:29), hu9F5VLv8 (SEQ ID NO:130, hu9F5VLv9 (SEQ ID NO:131), hu9F5VLv2_M51E (SEQ ID NO:61), hu9F5VLv2_M51D (SEQ ID NO:62), hu9F5VLv2_L27cD (SEQ ID NO:63), hu9F5VLv2_L27cG (SEQ ID NO:64), hu9F5VLv2_L27cS (SEQ ID NO:65), hu9F5VLv2_L27cE (SEQ ID NO:66), hu9F5VLv2_I30E (SEQ ID NO:67), hu9F5VLv2_I30K (SEQ ID NO:68), hu9F5VLv2_L27cT (SEQ ID NO:69), hu9F5VLv2_L27cN (SEQ ID NO:70), hu9F5VLv2_L27bD (SEQ ID NO:71), hu9F5VLv2_I30G, (SEQ ID NO:72), hu9F5VLv2_L33N (SEQ ID NO:73), hu9F5VLv2_L27cA (SEQ ID NO:74), hu9F5VLv2_L33T (SEQ ID NO:75), hu9F5VLv2_L33 S (SEQ ID NO:76), hu9F5VLv2_L33R (SEQ ID NO:77), hu9F5VLv2_I30Q (SEQ ID NO:78), hu9F5VLv2_L27bT (SEQ ID NO:79), hu9F5VLv2_T31G (SEQ ID NO:80), hu9F5VLv2_L27bQ (SEQ ID NO:81), hu9F5VLv2_L33G (SEQ ID NO:82), hu9F5VLv2_L27cP (SEQ ID NO:83), hu9F5VLv2_V78R (SEQ ID NO:84), hu9F5VLv2_I75D (SEQ ID NO:85), hu9F5VLv2_V78D (SEQ ID NO:86), hu9F5VLv2_V78E (SEQ ID NO:87), hu9F5VLv2_V78P (SEQ ID NO:88), hu9F5VLv2_V78K (SEQ ID NO:89), hu9F5VLv2_R77D (SEQ ID NO:90), hu9F5VLv2_V78G (SEQ ID NO:91), hu9F5VLv2_S76P (SEQ ID NO:92), hu9F5VLv2_I75P (SEQ ID NO:93), hu9F5VLv2_I75Q (SEQ ID NO:94), hu9F5VLv2_I75G (SEQ ID NO:95), hu9F5VLv2_L73P (SEQ ID NO:96), hu9F5VLv2_L73G (SEQ ID NO:97), hu9F5VLv2_V78Q (SEQ ID NO:98), hu9F5VLv2_S76G (SEQ ID NO:99), hu9F5VLv2_L92D (SEQ ID NO:100), hu9F5VLv2_Y86T (SEQ ID NO:101), hu9F5VLv2_L92E (SEQ ID NO:102), hu9F5VLv2_L92G (SEQ ID NO:103), hu9F5VLv2_L92Q (SEQ ID NO:104), hu9F5VLv2_L93G (SEQ ID NO:105), hu9F5VLv2_V85G (SEQ ID NO:106), hu9F5VLv2_L92T (SEQ ID NO:107), hu9F5VLv2_A89G (SEQ ID NO:108), hu9F5VLv8_DIM1 (SEQ ID NO:132), hu9F5VLv8_DIM2 (SEQ ID NO:133), hu9F5VLv8_DIM3 (SEQ ID NO:134), hu9F5VLv8_DIM4 (SEQ ID NO:135), hu9F5VLv8_DIM5 (SEQ ID NO:136), hu9F5VLv8_DIM6 (SEQ ID NO:137), hu9F5VLv8_DIM7 (SEQ ID NO:138), hu9F5VLv8_DIM8 (SEQ ID NO:139), hu9F5VLv8_DIM9 (SEQ ID NO:140), hu9F5VLv8_DIM 10 (SEQ ID NO:141), hu9F5VLv8_DIM11 (SEQ ID NO:142), hu9F5VLv8_DIM12 (SEQ ID NO:143), hu9F5VLv8_DIM13 (SEQ ID NO:144), hu9F5VLv8_DIM14 (SEQ ID NO:145), hu9F5VLv8_DIM15 (SEQ ID NO:146), hu9F5VLv8_DIM16 (SEQ ID NO:147), hu9F5VLv8_DIM17 (SEQ ID NO:148), hu9F5VLv8_DIM18 (SEQ ID NO:149), hu9F5VLv8_DIM19 (SEQ ID NO:150), hu9F5VLv8_DIM20 (SEQ ID NO:151), hu9F5VLv8_DIM21 (SEQ ID NO:152), hu9F5VLv8_DIM22 (SEQ ID NO:153), hu9F5VLv8_DIM23 (SEQ ID NO:154), hu9F5VLv8_DIM24 (SEQ ID NO:155), hu9F5VLv8_DIM25 (SEQ ID NO:156), hu9F5VLv8_DIM26 (SEQ ID NO:157), hu9F5VLv8_DIM27 (SEQ ID NO:158), hu9F5VLv8_DIM28 (SEQ ID NO:159), hu9F5VLv8_DIM29 (SEQ ID NO:160), hu9F5VLv8_DIM30 (SEQ ID NO:161), hu9F5VLv9_DIM1 (SEQ ID NO:162), hu9F5VLv9_DIM2 (SEQ ID NO:163), hu9F5VLv9_DIM4 (SEQ ID NO:164), hu9F5VLv9_DIM5 (SEQ ID NO:165), hu9F5VLv9_DIM8 (SEQ ID NO:166), hu9F5VLv9_DIM10 (SEQ ID NO:167), hu9F5VLv9_DIM11 (SEQ ID NO:168), hu9F5VLv9_DIM13 (SEQ ID NO:169), hu9F5VLv9_DIM19 (SEQ ID NO:170), or hu9F5VLv9_DIM20 (SEQ ID NO:171). In some such antibodies at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35,36, 37, 38, 39, 40, 41, 42, 43, 44, 45, 46, 47, 48, 49, 50, 51, 52, 53, 54, 55, 56, 57, 58, 59, 60, 61, or all 62 of the positions subject to backmutations or other mutations in SEQ ID NOs:15-22, 109-129, SEQ ID NOs:23-29, SEQ ID NOs:NOs:61-108, and SEQ ID NOs:130-171) are likewise backmutated or otherwise mutated.

In some humanized 9F5 antibodies, at least one of the following positions in the VH region is occupied by the amino acid as specified: H1 is occupied by E, H17 is occupied by T, H20 is occupied by I, H69 is occupied by M, H75 is occupied by T, H93 is occupied by T, H94 is occupied by T, and H109 is occupied by V. In some humanized 9F5 antibodies, positions H1, H17, H20, H69, H75, H94, and H109 are occupied by E, T, I, M, T, T, T, and V, respectively.

In some humanized 9F5 antibodies, at least one of the following positions in the VH region is occupied by the amino acid as specified: H66 is occupied by R and H81 is occupied by E. In some humanized 9F5 antibodies, positions H66 and H81 are occupied by R and E, respectively.

In some humanized 9F5 antibodies, at least one of the following positions in the VH region is occupied by the amino acid as specified: H23 is occupied by I and H83 is occupied by R. In some humanized 9F5 antibodies, positions H23 and H83 are occupied by K and R, respectively.

In some humanized 9F5 antibodies, at least one of the following positions in the VH region is occupied by the amino acid as specified: H43 is occupied by K, H51 is occupied by V, H76 is occupied by D, M80 is occupied by M, and H108 is occupied by L. In some humanized 9F5 antibodies, positions H43, H51, H76, H80, and H108 are occupied by K, V, D, M, and L, respectively.

In some humanized 9F5 antibodies, position H28 in the VH region is occupied by T.

In some humanized 9F5 antibodies, at least one of the following positions in the VH region is occupied by the amino acid as specified: H54 is occupied by D and H56 is occupied by E. In some humanized 9F5 antibodies, positions H54 and H56 are occupied by D and E, respectively.

In some humanized 9F5 antibodies, position H40 in the VH region is occupied by A. In some humanized 9F5 antibodies, at least one of the following positions in the VH region is occupied by the amino acid as specified: H5 is occupied by V, H11 is occupied by V, H12 is occupied by K, H38 is occupied by R, and H42 is occupied by G.

In some humanized 9F5 antibodies, positions H5, H11, H12, H38, and H42 are occupied by V, V, K, R, and G, respectively.

In some humanized 9F5 antibodies, at least one of the following positions in the VH region is occupied by the amino acid as specified: H1 is occupied by Q or E, H5 is occupied by Q or V, H11 is occupied by L or V, H12 is occupied by V or K, H17 is occupied by S or T, H20 is occupied by L or I, H23 is occupied by T or K, H28 is occupied by N or T, H38 is occupied by K, R or Q, H40 is occupied by R or A, H42 is occupied by E or G, H43 is occupied by Q or K, H48 is occupied by I or M, H51 is occupied by I or V, H54 is occupied by N or D, H56 is occupied by D or E, H66 is occupied by K or R, H69 is occupied by I or M, H75 is occupied by S or T, H76 is occupied by N or D, H79 is occupied by Y, Q, D, N, or G, H80 is occupied by L, M, P, D, G, or E, H81 is occupied by Q or E, H82 is occupied by L, P, K, R, E, or N, H82a is occupied by S or G, H82c is occupied by L, G, D, or S, H83 is occupied by T or R, H93 is occupied by A or T, H94 is occupied by S or T, H108 is occupied by T or L, H109 is occupied by L or V.

In some humanized 9F5 antibodies, positions H1, H17, H20, H69, H75, H93, H94, and H109 in the VH region are occupied by E, T, I, M, T, T, T, and V , respectively, as in hu9F5VHv2. In some humanized 9F5 antibodies, positions H1, H17, H20, H66, H69, H75, H81, H93, H94, and H109 in the VH region are occupied by E, T, I, R, M, T, E, T, T, and V, respectively, as in hu9F5VHv3. In some humanized 9F5 antibodies, positions H1, H17, H20, H23, H28, H66, H69, H75, H81, H83, H93, H94, and H109 in the VH region are occupied by E, T, I, K, T, R, M, T, E, R, T, T, and V, respectively, as in hu9F5VHv4. In some humanized 9F5 antibodies, positions H1, H17, H20, H23, H28, H43, H51, H54, H56, H66, H69, H75, H76, H80, H81, H83, H93, H94, H108, and H109 in the VH region are occupied by E, T, I, K, T, K, V, D, E, R, M, T, D, M, E, R, T, T, L, and V, respectively, as in hu9F5VHv5. In some humanized 9F5 antibodies, positions H1, H17, H20, H23, H28, H40, H43, H48, H51, H54, H56, H66, H69, H75, H76, H80, H81, H83, H93, H94, H108, and H109 in the VH region are occupied by E, T, I, K, T, A, K, M, V, D, E, R, M, T, D, M, E, R, T, T, L, and V, respectively, as in hu9F5VHv6. In some humanized 9F5 antibodies, positions H1, H5, H11, H12, H17, H20, H23, H38, H40, H42, H43, H51, H54, H56, H66, H69, H75, H76, H80, H81, H83, H93, H94, H108, and H109 in the VH region are occupied by E, V, V, K, T, I, K, R, A, G, K, V, D, E, R, M, T, D, M, E, R, T, T, L, and V, respectively, as in hu9F5VHv7. In some humanized 9F5 antibodies, positions H1, H5, H11, H12, H17, H20, H23, H38, H40, H42, H43, H51, H66, H69, H75, H76, H80, H81, H83, H93, H94, H108, and H109 in the VH region are occupied by E, V, V, K, T, I, K, R, A, G, K, V, R, M, T, D, M, E, R, T, T, L, and V, respectively, as in hu9F5VHv8. In some humanized 9F5 antibodies, positions H1, H5, H11, H12, H17, H20, H23, H38, H42, H43, H66, H69, H75, H80, H81, H83, H93, H94, H108, and H109 in the VH region are occupied by E, V, V, K, T, I, K, Q, G, K, R, M, T, M, E, R, T, T, L, and V, respectively, as in hu9F5VHv9. In some humanized 9F5 antibodies, the heavy chain variable region comprises the amino acid sequence of SEQ ID NO:127.

In some humanized 9F5 antibodies, positions H1, H5, H11, H12, H17, H20, H23, H38, H42, H43, H66, H69, H75, H80, H81, H83, H93, H94, H108, and H109 in the VH region are occupied by E, V, V, K, T, I, K, K, E, K, R, M, T, M, E, R, T, T, L, and V, respectively, as in hu9F5VHv10. In some humanized 9F5 antibodies, the heavy chain variable region comprises the amino acid sequence of SEQ ID NO:128.

In some humanized 9F5 antibodies, positions H1, H5, H11, H12, H17, H20, H23, H38, H42, H43, H66, H69, H75, H80, H81, H82c, H83, H93, H94, H108, and H109 in the VH region are occupied by E, V, V, K, T, I, K, K, E, K, R, M, T, M, E, G, R, T, T, L, and V, respectively, as in hu9F5VHv10_L82cG.

In some humanized 9F5 antibodies, at least one of the following positions in the VL region is occupied by the amino acid as specified: L7 is occupied by S, L8 is occupied by P, L15 is occupied by P, and L100 is occupied by Q. In some humanized 9F5 antibodies, positions L7, L8, L15, and L100 are occupied by S, P, P, and Q, respectively.

In some humanized 9F5 antibodies, position L66 in the VL region is occupied by G. In some humanized 9F5 antibodies, position L64 in the VL region is occupied by S.

In some humanized 9F5 antibodies, position L17 in VL region is occupied by E.

In some humanized 9F5 antibodies, at least one of the following positions in the VL region is occupied by the amino acid as specified: L11 is occupied by L, L51 is occupied by G, and L54 is occupied by R. In some humanized 9F5 antibodies, positions L11, L51, and L54 are occupied by L, G, and R, respectively.

In some humanized 9F5 antibodies, position L30 in the VL region is occupied by Y.

In some humanized 9F5 antibodies, at least one of the following positions in the VL region is occupied by the amino acid as specified: L3 is V or Q, L7 is A or S, L8 is A or P, L9 is F or L, L11 is N or L, L15 is L or P, L17 is T or E, L18 is S or P, L27b is L, D. T, or Q, L27c is L, D, G, S, E, T, N, A, P, or I, L30 is I, Y, E, K, G, or Q, L31 is T,N, or G, L33 is L, N, T, S, R, or G, L37 is L, Q, G, or I, L39 is R or K, L51 is M, G, E, D, K, or I, L54 is L, R, G, or T, L60 is N or D, L64 is G or S, L66 is E or G, L73 is L, P, or G, L74 is R or K, L75 is I, D, P, Q, or G, L76 is S, P, or G, L77 is SEQ ID NO:146 or D, L78 is V, R, D, E, P, K, G, or Q, L85 is V or G, L86 is Y or T, L89 is A or G, L92 is L, D, E, G, Q, T, or I, L93 is E or G, L100 is G or Q.

In some humanized 9F5 antibodies, positions L64 and L66 in the VL region are occupied by S and G, respectively, as in hu9F5VLv1. In some humanized 9F5 antibodies, positions L7, L8, L15, L64, L66, and L100 in the VL region are occupied by S, P, P, S, G, and Q, respectively, as in hu9F5VLv2. In some humanized 9F5 antibodies, positions L7, L8, L15, L17, L66, and L100 in the VL region are occupied by S, P, P, E, G, and Q, respectively, as in hu9F5VLv3.

In some humanized 9F5 antibodies, positions L7, L8, L11, L15, L17, L51, L54, L66, and L100 in the VL region are occupied by S, P, L, P, E, G, R, G, and Q, respectively, as in hu9F5VLv4. In some humanized 9F5 antibodies, the light chain variable region comprises the amino acid sequence of any of SEQ ID NOs:133, 135-137, 142-144, 149, 158, 159 and 168. In some humanized 9F5 antibodies, the light chain variable region comprises the amino acid sequence of SEQ ID NO:133. In some humanized 9F5 antibodies, the light chain variable region comprises the amino acid sequence of SEQ ID NO:137. In some humanized 9F5 antibodies, the light chain variable region comprises the amino acid sequence of SEQ ID NO:149. In some humanized 9F5 antibodies, the light chain variable region comprises the amino acid sequence of SEQ ID NO:159.

In some humanized 9F5 antibodies, positions L7, L8, L11, L15, L17, L30, L51, L54, L66, and L100 in the VL region are occupied by S, P, L, P, E, Y, G, R, G, and Q, respectively, as in hu9F5VLv5. In some humanized 9F5 antibodies, positions L7, L8, L11, L15, L17, L30, L51, L54, and L100 in the VL region are occupied by S, P, L, P, E, Y, G, R, and Q, respectively, as in hu9F5VLv6. In some humanized 9F5 antibodies, positions L7, L8, L9, L11, L15, L17, L18, L31, L39, L51, L54, L60, L66, L74, and L100 in the VL region are occupied by S, P, L, L, P, E, P, N, K, G, R, D, G, K, and Q, respectively, as in hu9F5VLv7.

In some humanized 9F5 antibodies, positions L7, L8, L11, L15, L17, L39, L64, L66, L74, and L100 in the VL region are occupied by S, P, L, P, E, K, S, G, K and Q, respectively, as in hu9F5VLv8. In some humanized 9F5 antibodies, position L3 in the VL region is occupied by Q. In some humanized 9F5 antibodies, position L27c in the VL region is occupied by D, G, I, L or S, position L37 in the VL region is occupied by G, I, L, or Q, position L51 in the VL region is occupied by E, G, I, K or M, position L54 in the VL region is occupied by G, L, R or T, and position L92 in the VL region is occupied by G, I or L. In some humanized 9F5 antibodies, position L27c in the VL region is occupied by D or S, position L37 in the VL region is occupied by G, L or Q, position L51 in the VL region is occupied by G or K, position L54 in the VL region is occupied by R, and position L92 in the VL region is occupied by I.

In some humanized 9F5 antibodies, position L27c in the VL region is occupied by D, position L37 in the VL region is occupied by G, and position L51 in the VL region is occupied by G. In some humanized 9F5 antibodies, the heavy chain variable region has an amino acid sequence comprising SEQ ID NO:127 and the light chain variable region has an amino acid sequence comprising SEQ ID NO:149.

In some humanized 9F5 antibodies, position L27c in the VL region is occupied by D, position L37 in the VL region is occupied by Q, and position L51 in the VL region is occupied by G. In some humanized 9F5 antibodies, the heavy chain variable region has an amino acid sequence comprising SEQ ID NO:127 and the light chain variable region has an amino acid sequence comprising SEQ ID NO:137.

In some humanized 9F5 antibodies, position L27c in the VL region is occupied by S, position L37 in the VL region is occupied by L and position L51 in the VL region is occupied by G. In some humanized 9F5 antibodies, the heavy chain variable region has an amino acid sequence comprising SEQ ID NO:127 and the light chain variable region has an amino acid sequence comprising SEQ ID NO:159.

In some humanized 9F5 antibodies, position L27c in the VL region is occupied by D, position L37 in the VL region is occupied by Q, and position L51 in the VL region is occupied by K. In some humanized 9F5 antibodies, the heavy chain variable region has an amino acid sequence comprising SEQ ID NO:127 and the light chain variable region has an amino acid sequence comprising SEQ ID NO:138.

In some humanized 9F5 antibodies, position L27c in the VL region is occupied by S, position L37 in the VL region is occupied by Q, and position L51 in the VL region is occupied by G. In some humanized 9F5 antibodies, the heavy chain variable region has an amino acid sequence comprising SEQ ID NO:127 and the light chain variable region has an amino acid sequence comprising SEQ ID NO:133.

In some humanized 9F5 antibodies, positions L7, L8, L11, L15, L17, L39, L60, L64, L66, L74, and L100 in the VL region are occupied by, respectively S, P, L, P, E, K, D, S, G, K, and Q, as in hu9F5VLv9. In some humanized 9F5 antibodies, position L3 in the VL region is occupied by Q.

In some humanized 9F5 antibodies, position L27c in the VL region is occupied by G or S, position L37 in the VL region is occupied by G, I or Q, position L51 in the VL region is occupied by G, I or K, position L54 in the VL region is occupied by G or R, and position L92 in the VL region is occupied by G, I or L. In some humanized 9F5 antibodies, position L27c in the VL region is occupied by G, position L37 in the VL region is occupied by G, position L51 in the VL region is occupied by G, and position L54 in the VL region is occupied by R. In some humanized 9F5 antibodies, position L92 in the VL region is occupied by I. In some humanized 9F5 antibodies, the heavy chain variable region has an amino acid sequence comprising SEQ ID NO:129 and the light chain variable region has an amino acid sequence comprising SEQ ID NO:168.

The light chain variable region of any of the above referenced antibodies can be modified to further reduce immunogenicity. For example, in some of the humanized antibodies position L27b in the VL region is occupied by D, T or Q; position L27c in the VL region is occupied by D, G, S, E, T, N, A, I, or P; position L30 in the VL region is occupied by E, K, G or Q; position L31 in the VL region is occupied by G; position L33 in the VL region is occupied by N, T, S, R or G; position L37 in the VL region is occupied by Q, G, or I, position L51 in the VL region is occupied by E, D, G, K, or I; position L54 in the VL region is occupied by G, R, or T, position L60 in the VL region is occupied by D, position L73 in the VL region is occupied by P or G; position L75 in the VL region is occupied by D, P, Q or G; position L76 in the VL region is occupied by P or G; position L77 in the VL region is occupied by D; position L78 in the VL region is occupied by R, D, E, P, K, G or Q; position L85 in the VL region is occupied by G; position L86 in the VL region is occupied by T; position L89 in the VL region is occupied by G; position L92 in the VL region is occupied by D, E, G, Q, I, or T; and/or position L93 in the VL region is occupied by G (Kabat numbering).

In some humanized 9F5 antibodies, position L51 in the VL region is occupied by E, as in hu9F5VLv2_M51E. In some humanized 9F5 antibodies, position L51 in the VL region is occupied by D, as in hu9F5VLv2_M51D. In some humanized 9F5 antibodies, position L27c in the VL region is occupied by D, as in hu9F5VLv2_L27cD. In some humanized 9F5 antibodies, position L27c in the VL region is occupied by G, as in hu9F5VLv2_L27cG. In some humanized 9F5 antibodies, position L27c in the VL region is occupied by S, as in hu9F5VLv2_L27cS. In some humanized 9F5 antibodies, position L27c in the VL region is occupied by E, as in hu9F5VLv2_L27cE. In some humanized 9F5 antibodies, position L30 in the VL region is occupied by E, as in hu9F5VLv2_I30E. In some humanized 9F5 antibodies, position L30 in the VL region is occupied by K, as in hu9F5VLv2_I30K. In some humanized 9F5 antibodies, position L27c in the VL region is occupied by T, as in hu9F5VLv2_L27cT. In some humanized 9F5 antibodies, position L27c in the VL region is occupied by N, as in hu9F5VLv2_L27cN)

In some humanized 9F5 antibodies, position L27b in the VL region is occupied by D, as in hu9F5VLv2_L27bD. In some humanized 9F5 antibodies, position L30 in the VL region is occupied by G, as in hu9F5VLv2_I30G. In some humanized 9F5 antibodies, position L33 in the VL region is occupied by N, as in hu9F5VLv2_L33N. In some humanized 9F5 antibodies, position L27c in the VL region is occupied by A, as in hu9F5VLv2_L27cA. In some humanized 9F5 antibodies, position L33 in the VL region is occupied by T, as in hu9F5VLv2_L33T. In some humanized 9F5 antibodies, position L33 in the VL region is occupied by S, as in hu9F5VLv2_L33S. In some humanized 9F5 antibodies, position L33 in the VL region is occupied by R, as in hu9F5VLv2_L33R. In some humanized 9F5 antibodies, position L30 in the VL region is occupied by Q, as in hu9F5VLv2_I30Q. In some humanized 9F5 antibodies, position L27b in the VL region is occupied by T, as in hu9F5VLv2_L27bT. In some humanized 9F5 antibodies, position L31 in the VL region is occupied by G, as in hu9F5VLv2_T31G.

In some humanized 9F5 antibodies, position L27b in the VL region is occupied by Q, as in hu9F5VLv2_L27bQ) In some humanized 9F5 antibodies, position L33 in the VL region is occupied by G, as in hu9F5VLv2_L33G. In some humanized 9F5 antibodies, position L27c in the VL region is occupied by P, as in hu9F5VLv2_L27cP. In some humanized 9F5 antibodies, position L78 in the VL region is occupied by R, as in hu9F5VLv2_V78R. In some humanized 9F5 antibodies, position L75 in the VL region is occupied by D, as in hu9F5VLv2_I75D. In some humanized 9F5 antibodies, position L78 in the VL region is occupied by D, as in hu9F5VLv2_V78D. In some humanized 9F5 antibodies, position L78 in the VL region is occupied by E, as in hu9F5VLv2_V78E. In some humanized 9F5 antibodies, position L78 in the VL region is occupied by P, as in hu9F5VLv2_V78P. In some humanized 9F5 antibodies, position L78 in the VL region is occupied by K, as in hu9F5VLv2_V78K. In some humanized 9F5 antibodies, position L77 in the VL region is occupied by D, as in hu9F5VLv2_R77D.

In some humanized 9F5 antibodies, position L78 in the VL region is occupied by G, as in hu9F5VLv2_V78G. In some humanized 9F5 antibodies, position L76 in the VL region is occupied by P, as in hu9F5VLv2_S76P. In some humanized 9F5 antibodies, position L75 in the VL region is occupied by P, as in hu9F5VLv2_I75P. In some humanized 9F5 antibodies, position L75 in the VL region is occupied by Q, as in hu9F5VLv2_I75Q. In some humanized 9F5 antibodies, position L75 in the VL region is occupied by G, as in hu9F5VLv2_I75G. In some humanized 9F5 antibodies, position L73 in the VL region is occupied by P, as in hu9F5VLv2_L73P. In some humanized 9F5 antibodies, position L73 in the VL region is occupied by G, as in hu9F5VLv2_L73G. In some humanized 9F5 antibodies, position L78 in the VL region is occupied by Q, as in hu9F5VLv2_V78Q. In some humanized 9F5 antibodies, position L76 in the VL region is occupied by G, as in hu9F5VLv2_S76G. In some humanized 9F5 antibodies, position L92 in the VL region is occupied by D, as in hu9F5VLv2_L92D.

In some humanized 9F5 antibodies, position L86 in the VL region is occupied by T, as in hu9F5VLv2_Y86T. In some humanized 9F5 antibodies, position L92 in the VL region is occupied by E, as in hu9F5VLv2_L92E. In some humanized 9F5 antibodies, position L92 in the VL region is occupied by G, as in hu9F5VLv2_L92G. In some humanized 9F5 antibodies, position L92 in the VL region is occupied by Q, as in hu9F5VLv2_L92Q. In some humanized 9F5 antibodies, position L93 in the VL region is occupied by G, as in hu9F5VLv2_L93G). In some humanized 9F5 antibodies, position L85 in the VL region is occupied by G, as in hu9F5VLv2_V85G. In some humanized 9F5 antibodies, position L92 in the VL region is occupied by T, as in hu9F5VLv2_L92T. In some humanized 9F5 antibodies, position L89 in the VL region is occupied by G, as in hu9F5VLv2_A89G.

In some humanized 9F5 antibodies, positions L3, L27c, L37, L51, L54, and L92 in the VL region are occupied by Q, S, Q, G, G, and I, respectively, as in hu9F5VLv8_V3Q, L27cS, L37Q, M51G, L54G, L92I, also known as hu9F5VLv8_DIM1. In some humanized 9F5 antibodies, positions L3, L27c, L37, L51, L54, and L92 in the VL region are occupied by Q, S, Q, G, R, and I, hu9F5VLv8_V3Q, L27cS, L37Q, M51G, L54R, L92I, also known as hu9F5VLv8_DIM2. In some humanized 9F5 antibodies, positions L3, L27c, L37, L51, L54, and L92 in the VL region are occupied by Q, S, Q, G, T, and I, respectively, as in hu9F5VLv8_V3Q, L27cS, L37Q, M51G, L54T, L92I, also known as hu9VLv8_DIM3. In some humanized 9F5 antibodies, positions L3, L27c, L37, L51, L54, and L92 in the VL region are occupied by Q, S, Q, G, R, and G, respectively, as in hu9F5VLv8_V3Q, L27cS, L37Q, M51G, L54R, L92G, also known as hu9F5VLv8_DIM4. In some humanized 9F5 antibodies, positions L3, L27c, L37, L51, L54, and L92 in the VL region are occupied by Q, G, Q, G, R, and I, respectively, as in hu9F5VLv8_V3Q, L27cG, L37Q, M51G, L54R, L92I, also known as hu9F5VLv8_DIM5.

In some humanized 9F5 antibodies, positions L3, L27c, L37, L51, L54, and L92 in the VL region are occupied by Q, D, Q, G, R, and I, respectively, as in hu9F5VLv8_V3Q, L27cD, L37Q, M51G, L54R, L92I, also known as hu9F5VLv8_DIM6. In some humanized 9F5 antibodies, positions L3, L27c, L37, L51, L54, and L92 in the VL region are occupied by Q, D, Q, K, R, and I, respectively, as in hu9F5VLv8_V3Q, L27cD, L37Q, M51K, L54R, L92I, also known as hu9F5 v8_DIM7. In some humanized 9F5 antibodies, positions L3, L27c, L37, L51, L54, and L92 in the VL region are occupied by Q, G, Q, K, R, and I, respectively, as in hu9F5VLv8_V3Q, L27cG, L37Q, M51K, L54R, L92I, also known as hu9F5VLv8_DIM8. In some humanized 9F5 antibodies, positions L3, L27c, L37, L51, L54, and L92 in the VL region are occupied by Q. G, Q, K, G, and I, respectively, as in hu9F5VLv8_V3Q, L27cG, L37Q, M51K, L54G, L92I, also known as hu9F5VLv8_DIM9. In some humanized 9F5 antibodies, positions L3, L27c, L37, L51, L54, and L92 in the VL region are occupied by Q, S, Q, K, G, and I, respectively, as in hu9F5VLv8_V3Q, L27cS, L37Q, M51K, L54G, L92I, also known as hu9F5VLv8_DIM10

In some humanized 9F5 antibodies, positions L3, L27c, L37, L51, L54, and L92 in the VL region are occupied by Q, G, G, G, R, and I, respectively, as in hu9F5VLv8_V3Q, L27cG, L37G, M51G, L54R, L92I, also known as hu9F5VLv8_DIM11. In some humanized 9F5 antibodies, positions L3, L27c, L37, L51, L54, and L92 in the VL region are occupied by Q, G, G, G, R, and G, respectively, as in hu9F5VLv8_V3Q, L27cG, L37G, M51G, L54R, L92G, also known as hu9F5VLv8_DIM12. In some humanized 9F5 antibodies, positions L3, L27c, L37, L51, and L54 in the VL region are occupied by Q, G, G, G, and R, respectively, as in hu9F5VLv8_V3Q, L27cG, L37G, M51G, L54R, also known as hu9F5VLv8_DIM13. In some humanized 9F5 antibodies, positions L3, L27c, L37, L51, L54, and L92 in the VL region are occupied by Q, G, G, G, T, and I, respectively, as in hu9F5VLv8_V3Q, L27cG, L37G, M51G, L54T, L92I, also known as hu9F5 VLv8_DIM14. In some humanized 9F5 antibodies, positions L3, L27c, L37, L51, L54, and L92 in the VL region are occupied by Q, G, G, G, T, and G, respectively, as in hu9F5VLv8_V3Q, L27cG, L37G, M51G, L54T, L92G, also known as hu9F5VLv8_DIM15.

In some humanized 9F5 antibodies, positions L3, L27c, L37, L51, and L54 in the VL region are occupied by Q, G, G, G, and T, respectively, as in hu9F5VLv8_V3Q, L27cG, L37G, M51G, L54T, also known as hu9F5VLv8_DIM16. In some humanized 9F5 antibodies, positions L3, L27c, L37, L51, L54, and L92 in the VL region are occupied by Q, S, G, G, T, and I, respectively, as in hu9F5VLv8_V3Q, L27cS, L37G, M51G, L54T, L92I, also known as hu 9F5VLv8_DIM17 In some humanized 9F5 antibodies, positions L3, L27c, L37, L51, L54, and L92 in the VL region are occupied by Q, D, G, G, R, and I, respectively, as in hu9F5VLv8_V3Q, L27cD, L37G, M51G, L54R, L92I, also known as hu9F5VLv8_DIM18. In some humanized 9F5 antibodies, positions L3, L27c, L37, L51, L54, and L92 in the VL region are occupied by Q, S, I, I, R, and I, respectively, as in hu9F5VLv8_V3Q, L27cS, L37I, M51I, L54R, L92I, also known as hu9F5VLv8_DIM19. In some humanized 9F5 antibodies, positions L3, L27c, L37, L51, L54, and L92 in the VL region are occupied by Q, S, Q, I, G, and I, respectively, as in hu9F5VLv8_V3Q, L27cS, L37Q, M51I, L54G, L92I, also known as hu9F5VLv8_DIM 20.

In some humanized 9F5 antibodies, positions L3, L27c, L37, L51, and L54 in the VL region are occupied by Q, S, Q, I, and G, respectively, as in hu9F5VLv8_V3Q, L27cS, L37Q, M51I, L54G, also known as hu9F5VLv8_DIM21. In some humanized 9F5 antibodies, positions L3, L27c, L37, L51, L54, and L92 in the VL region are occupied by Q, S, Q, E, R, and I, respectively, as in hu9F5VLv8_V3Q, L27cS, L37Q, M51E, L54R, L92I, also known as hu9F5VLv8_DIM22 In some humanized 9F5 antibodies, positions L3, L27c, L37, L51, L54, and L92 in the VL region are occupied by Q, G, Q, E, G, and I, respectively, as in hu9F5VLv8_V3Q, L27cG, L37Q, M51E, L54G, L92I, also known as hu9F5VLv8_DIM23. In some humanized 9F5 antibodies, positions L3, L27c, L37, L51, L54, and L92L in the VL region are occupied by Q, G, I, E, R, and I, respectively, as in hu9F5VLv8_V3Q, L27cG, L37I, M51E, L54R, L92I, also known as hu9F5VLv8_DIM24. In some humanized 9F5 antibodies, positions L3, L27c, L37, L51, L54, and L92 in the VL region are occupied by Q, G, I, E, R. and G, respectively, as in hu9F5VLv8_V3Q, L27cG, L37I, M51E, L54R, L92G, also known as hu9F5VLv8_DIM 25.

In some humanized 9F5 antibodies, positions L3, L27c, L37, L51, and L54 in the VL region are occupied by Q, I, I, E, and R, respectively, as in hu9F5VLv8_V3Q, L27cI, L37I, M51E, L54R, also known as hu9F5VLv8_DIM26 In some humanized 9F5 antibodies, positions L3, L37, L51, L54, and L92 in the VL region are occupied by Q, Q, G, R, and I, respectively, as in hu9F5VLv8_V3Q, L37Q, M51G, L54R, L92I, also known as hu9F5VLv8_DIM27. In some humanized 9F5 antibodies, positions L3, L27c, L51, L54, and L92 in the VL region are occupied by Q, S, G, R, and I, respectively, as in hu9F5VLv8_V3Q, L27cS, M51G, L54R, L92I, also known as hu9F5VLv8_DIM28. In some humanized 9F5 antibodies, positions L3, L27c, L37, L54, and L92 in the VL region are occupied by Q, S, Q, R, and I, respectively, as in hu9F5VLv8_V3Q, L27cS, L37Q, L54R, L92I, also known as hu9F5VLv8_DIM29. In some humanized 9F5 antibodies, positions L3, L27c, L37, L51, and L92 in the VL region are occupied by Q, S, Q, G, and I, respectively, as in hu9F5VLv8_V3Q, L27cS, L37Q, M51G, L92I, also known as hu9F5VLv8_DIM30.

In some humanized 9F5 antibodies, positions L3, L27c, L37, L51, L54, and L92 in the VL region are occupied by Q, S, Q, G, G, and I, respectively, as in hu9F5VLv9_V3Q, L27cS, L37Q, M51G, L54G, L92I, also known as hu9F5VLv9_DIM1. In some humanized 9F5 antibodies, positions L3, L27c, L37, L51, L54, and L92in the VL region are occupied by Q, S, Q, G, R, and I, respectively, as in hu9F5VLv9_V3Q, L27cS, L37Q, M51G, L54R, L92I, also known as hu9F5VLv9_DIM2. In some humanized 9F5 antibodies, positions L3, L27c, L37, L51, L54, and L92 in the VL region are occupied by Q, S, Q, G, R, and G, respectively, as in hu9F5VLv9_V3Q, L27cS, L37Q, M51G, L54R, L92G, also known as hu9F5VLv9_DIM4). In some humanized 9F5 antibodies, positions L3, L27c, L37, L51, L54, and L92 in the VL region are occupied by Q, G, Q, G, R, and I, respectively, as in hu9F5VLv9_V3Q, L27cG, L37Q, M51G, L54R, L92I, also known as hu9F5 VLv9_DIM5. In some humanized 9F5 antibodies, positions L3, L27c, L37, L51, L54, and L92 in the VL region are occupied by Q, G, Q, K, R, and I, respectively, as in hu9F5VLv9_V3Q, L27cG, L37Q, M51K, L54R, L92I, also known as hu9F5VLv9_DIM8.

In some humanized 9F5 antibodies, positions L3, L27c, L37, L51, L54, and L92 in the VL region are occupied by Q, S, Q, K, G, and I, respectively, as in hu9F5VLv9_V3Q, L27cS, L37Q, M51K, L54G, L92I, also known as hu9F5VLv9_DIM10. In some humanized 9F5 antibodies, positions L3, L27c, L37, L51, L54, and L92 in the VL region are occupied by Q, G, G, G, R, and I, respectively, as in hu9F5VLv9_V3Q, L27cG, L37G, M51G, L54R, L92I, also known as hu9F5VLv9_DIM11. In some humanized 9F5 antibodies, positions L3, L27c, L37, L51, and L54 in the VL region are occupied by Q, G, G, G, and R, respectively, as in hu9F5VLv9_V3Q, L27cG, L37G, M51G, L54R, also known as hu9F5VLv9_DIM13. In some humanized 9F5 antibodies, positions L3, L27c, L37, L51, L54, and L92 in the VL region are occupied by Q, S, I, I, R, and I, respectively, as in hu9F5VLv9_V3Q, L27cS, L37I, M51I, L54R, L92I, also known as hu9F5VLv9_DIM19. in some humanized 9F5 antibodies, positions L3, L27c, L37, L51, L54, and L92 in the VL region are occupied by Q, S, Q, I, G, and I, respectively, as in hu9F5VLv9_V3Q, L27cS, L37Q, M51I, L54G, L92I, also known as hu9F5VLv9_DIM20.

The heavy chain variable region of any of the above referenced antibodies can also be modified to further reduce immunogenicity. For example, in some of the humanized antibodies, position H79 is occupied by D, N, G or Q; position H80 is occupied by P, D, E or G; position H82 is occupied by P, K, R, E or N; position H82a is occupied by G; and/or position H82c is occupied by G, D or S.

In some humanized 9F5 antibodies, position H80 is occupied by P, as in hu9F5VHv4_L80P. In some humanized 9F5 antibodies, position H80 is occupied by D, as in hu9F5VHv4_L80D. In some humanized 9F5 antibodies, position H82c is occupied by G, as in hu9F5VHv4_L82cG and in hu9F5VHv10_L82cG. In some humanized 9F5 antibodies, position H82c is occupied by D, as in hu9F5VHv4_L82cD. In some humanized 9F5 antibodies, position H82 is occupied by P, as in hu9F5VHv4_L82P. In some humanized 9F5 antibodies, position H80 is occupied by G, as in hu9F5VHv4_L80G. In some humanized 9F5 antibodies, position H82 is occupied by K, as in hu9F5VHv4_L82K. In some humanized 9F5 antibodies, position H82 is occupied by R, as in hu9F5VHv4_L82R. In some humanized 9F5 antibodies, position H82 is occupied by E, as in hu9F5VHv4_L82E. In some humanized 9F5 antibodies, position H82 is occupied by N, as in hu9F5VHv4_L82N.

In some humanized 9F5 antibodies, position H79 is occupied by D, as in hu9F5VHv4_Y79D. In some humanized 9F5 antibodies, position H79 is occupied by N, as in hu9F5VHv4_Y79N. In some humanized 9F5 antibodies, position H79 is occupied by G, as in hu9F5VHv4_Y79G. In some humanized 9F5 antibodies, position H80 is occupied by E, as in hu9F5VHv5_M80E. In some humanized 9F5 antibodies, position H80 is occupied by G, as in hu9F5VHv5_M80G. In some humanized 9F5 antibodies, position H82c is occupied by S, as in hu9F5VHv4_L82cS. In some humanized 9F5 antibodies, position H79 is occupied by Q, as in hu9F5VHv4_Y79Q. In some humanized 9F5 antibodies, position H82a is occupied by G, as in hu9F5VHv4_S82aG.

In some humanized 9F5 antibodies, the variable heavy chain has ≥85% identity to human sequence. In some humanized 9F5 antibodies, the variable light chain has ≥85% identity to human sequence. In some humanized 9F5 antibodies, each of the variable heavy chain and variable light chain has ≥85% identity to human germline sequence. In some humanized 9F5 antibodies, the three heavy chain CDRs are as defined by Kabat/Chothia Composite (SEQ ID NOs:8, 9, and 10) and the three light chain CDRs are as defined by Kabat/Chothia Composite (SEQ ID NOs:12, 13, and 14); provided that position H28 is occupied by N or T, position H51 is occupied by I or V, position H54 is occupied by N or D, position H56 is occupied by D or E, position L27b is occupied by L, D, T, or Q, position L27c is occupied by L, D, G, S, E, T, N, A, P, or I, position L30 is occupied by I, Y, E, K, G, or Q, position L31 is occupied by T, N, or G, position L33 is occupied by L N, T, S, R, or G, position L51 is occupied by M, G, E, D, K, or I, position L54 is occupied by L, R, G, or T, position L89 is occupied by A or G, position L92 is occupied by L, D, E, G, Q, T, or I, and position L93 is occupied by E or G.

In some humanized 9F5 antibodies, Kabat-Chothia Composite CDR-H1 has an amino acid sequence comprising SEQ ID NO:50. In some humanized 9F5 antibodies, Kabat CDR-H2 has an amino acid sequence comprising SEQ ID NO:51 or SEQ ID NO:52. In some humanized 9F5 antibodies, Kabat CDR-L1 has an amino acid sequence comprising SEQ ID NO:53 or SEQ ID NO:54. In some humanized 9F5 antibodies, Kabat CDR-L2 has an amino acid sequence comprising SEQ ID NO:55. In some humanized 9F5 antibodies, Kabat CDR-L1 has an amino acid sequence selected from the group consisting of SEQ ID NOs:172-193. In some humanized 9F5 antibodies, Kabat CDR-L2 has an amino acid sequence selected from the group consisting of SEQ ID NOs:194-205. In some humanized 9F5 antibodies, Kabat CDR-L3 has an amino acid sequence selected from the group consisting of SEQ ID NOs:206-213.

Exemplary humanized antibodies include humanized forms of the mouse 10C12, designated Hu10C12.

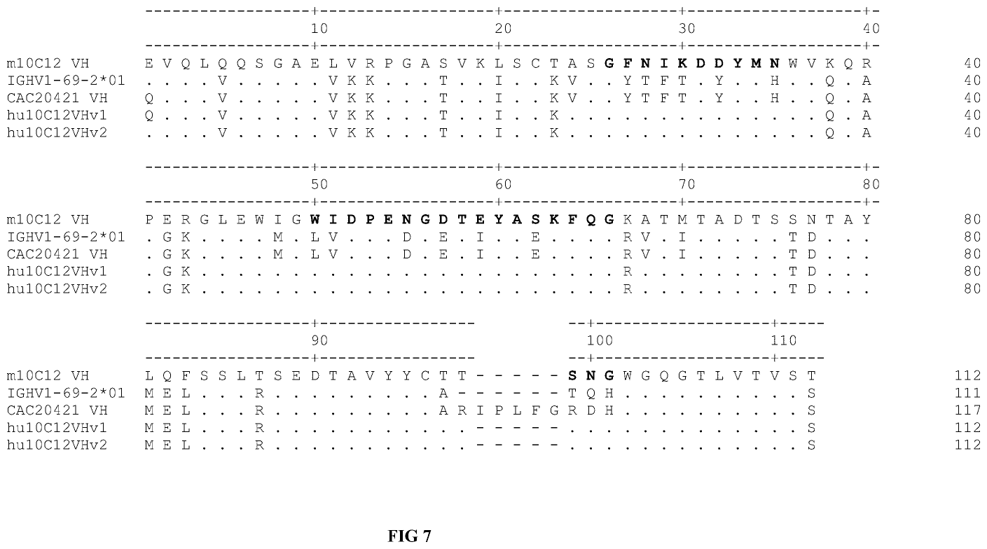

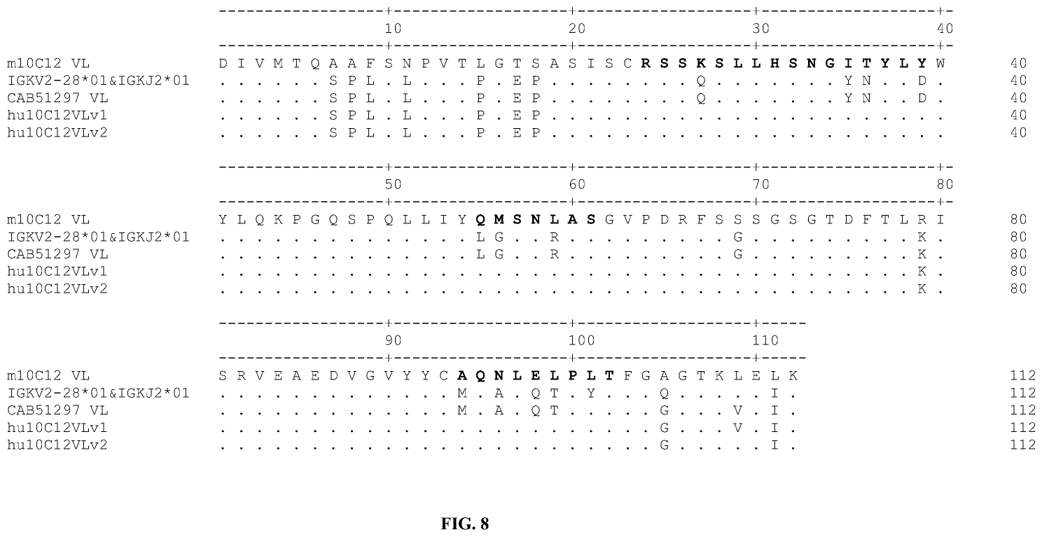

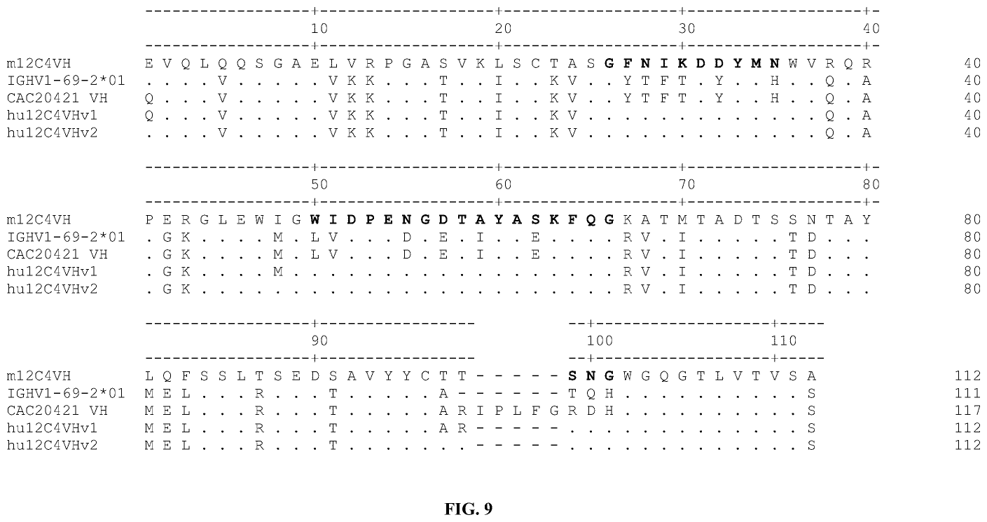

The mouse antibody 10C12 comprises mature heavy and light chain variable regions having amino acid sequences comprising SEQ ID NO:7 and SEQ ID NO:11, respectively. The invention provides 2 exemplified humanized mature heavy chain variable regions: hu10C12VHv1 and hu10C12VHv2. The invention further provides 2 exemplified mature light chain variable regions hu10C12VLv1 and hu10C12VLv2. FIGS. 7 and 8 show alignments of the heavy chain variable region and light chain variable region, respectively, of murine 10C12 and various humanized antibodies.

For reasons such as possible influence on CDR conformation and/or binding to antigen, mediating interaction between heavy and light chains, interaction with the constant region, being a site for desired or undesired post-translational modification, being an unusual residue for its position in a human variable region sequence and therefore potentially immunogenic, reducing aggregation potential, and other reasons, the following 9 variable region framework positions were considered as candidates for substitutions in the 2 exemplified human mature light chain variable regions and the 2 exemplified human mature heavy chain variable regions, as further specified in the examples: L64 (G64S), L104 (V104L), H1 (Q1E), H24 (V24A), H48 (M48I), H67 (V67A), H69 (I69M), H93 (A93T), and H94 (R94T).

Here, as elsewhere, the first-mentioned residue is the residue of a humanized antibody formed by grafting Kabat CDRs or a composite Chothia-Kabat CDR in the case of CDR-H1 into a human acceptor framework, and the second-mentioned residue is a residue being considered for replacing such residue. Thus, within variable region frameworks, the first mentioned residue is human, and within CDRs, the first mentioned residue is mouse.

Exemplified antibodies include any permutations or combinations of the exemplified mature heavy and light chain variable regions hu10C12VHv1/hu10C12VLv1, hu10C12VHv1/hu10C12VLv2, hu10C12VHv2/hu10C12VLv1, hu10C12VHv2/hu10C12VLv2.

Exemplified antibodies include any permutations or combinations of the exemplified mature heavy chain variable regions hu10C12VHv1 (SEQ ID NO:214) and hu10C12VHv2 (SEQ ID NO:215), with any of the exemplified mature light chain variable regions hu10C12VLv1 (SEQ ID NO:216) and hu10C12VLv2 (SEQ ID NO:217).

The invention provides variants of the 10C12 humanized antibody in which the humanized mature heavy chain variable region shows at least 90%, 95%, 96%, 97%, 98%, or 99% identity to hu10C12VHv1 (SEQ ID NO:214) or hu10C12VHv2 (SEQ ID NO:215) and the humanized mature light chain variable region shows at least 90%, 95%, 96%, 97%, 98%, or 99% identity to hu10C12VLv1 (SEQ ID NO:216) or hu10C12VLv2 (SEQ ID NO:217). In some such antibodies at least 1, 2, 3, 4, 5, 6, 7, 8, or all 9 of the backmutations or other mutations in SEQ ID NOs:214-215, and SEQ ID NOs:216-217) are retained.

In some humanized 10C12 antibodies, at least one of the following positions in the VH region is occupied by the amino acid as specified: H24 is occupied by A, H48 is occupied by I, H67 is occupied by A, H69 is occupied by M, H93 is occupied by T, and H94 is occupied by T. In some humanized 10C12 antibodies, positions H24, H48, H67, H69, H93, and H94 are occupied by A, I, A, M, T, and T, respectively.

In some humanized 10C12 antibodies, at least one of the following positions in the VH region is occupied by the amino acid as specified: H1 is occupied by Q or E, H24 is occupied by A, H48 is occupied by I, H67 is occupied by A, H69 is occupied by M, H93 is occupied by T, H94 is occupied by T.

In some humanized 10C12 antibodies, positions H24, H48, H67, H69, H93, and H94 are occupied by A, I, A, M, T, and T, respectively, as in hu10C12VHv1. In some humanized 10C12 antibodies, positions H1, H24, H48, H67, H69, H93, and H94 are occupied by E, A, I, A, M, T, and T, respectively, as in hu10C12VHv2.

In some humanized 10C12 antibodies, position L64 in the VL region is occupied by S.

In some humanized 10C12 antibodies, at least one of the following positions in the VL region is occupied by the amino acid as specified: L64 is S, L104 is V or L.

In some humanized 10C12 antibodies, position L64 and L104 in the VL region is occupied by S, as in hu10C12VLv1. In some humanized 10C12 antibodies, positions L64 and L104 in the VL region are occupied by S and L, respectively, as in hu10C12VLv2.

In some humanized 10C12 antibodies, the variable heavy chain has ≥85% identity to human sequence. In some humanized 10C12 antibodies, the variable light chain has ≥85% identity to human sequence. In some humanized 10C12 antibodies, each of the variable heavy chain and variable light chain has ≥85% identity to human germline sequence. In some humanized 10C12 antibodies, the three heavy chain CDRs are as defined by Kabat/Chothia Composite (SEQ ID NOs:8, 9, and 10) and the three light chain CDRs are as defined by Kabat/Chothia Composite (SEQ ID NOs:12, 13, and 14).

Exemplary humanized antibodies include humanized forms of the mouse 12C4, designated Hu12C4.

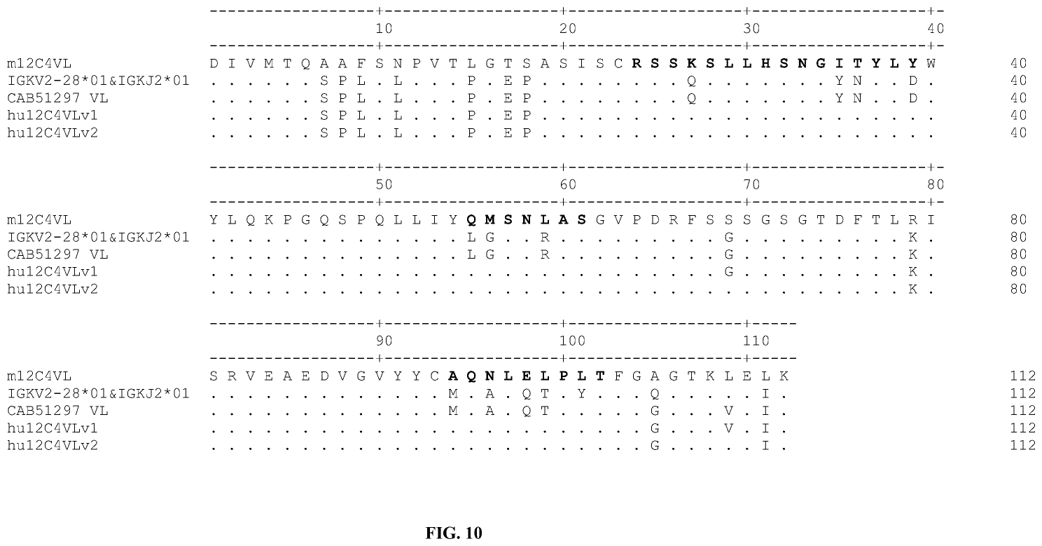

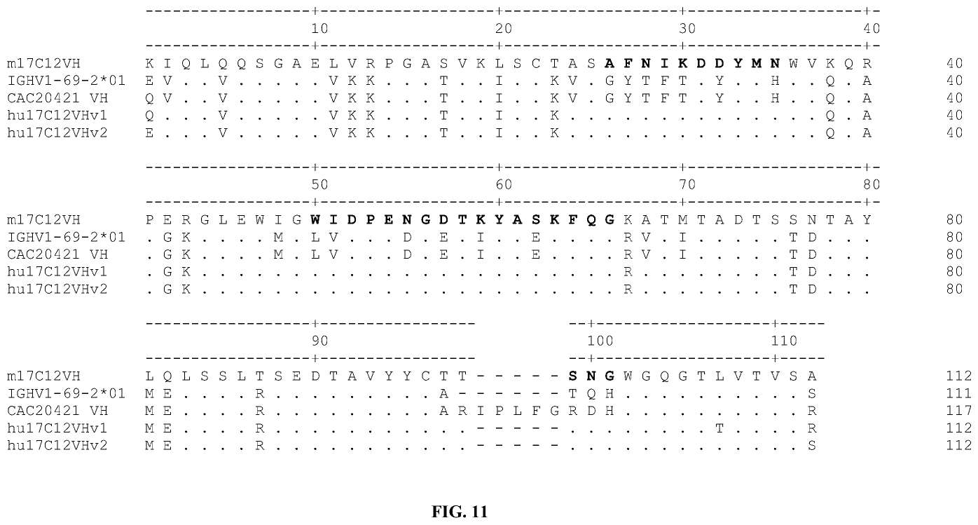

The mouse antibody 12C4 comprises mature heavy and light chain variable regions having amino acid sequences comprising SEQ ID NO:219 and SEQ ID NO:11, respectively. The invention provides 2 exemplified humanized mature heavy chain variable regions: hu12C4VHv1 and hu12C4VHv2. The invention further provides 2 exemplified mature light chain variable regions hu12C4VLv1 and hu12C4VLv2. FIGS. 9 and 10 show alignments of the heavy chain variable region and light chain variable region, respectively, of murine 12C4 and various humanized antibodies.

For reasons such as possible influence on CDR conformation and/or binding to antigen, mediating interaction between heavy and light chains, interaction with the constant region, being a site for desired or undesired post-translational modification, being an unusual residue for its position in a human variable region sequence and therefore potentially immunogenic, getting aggregation potential, and other reasons, the following 6 variable region framework positions were considered as candidates for substitutions in the 2 exemplified human mature light chain variable regions and the 2 exemplified human mature heavy chain variable regions, as further specified in the examples: L64 (G64S), L104 (V104L), H1 (Q1E), H48 (M48I), H93 (A93T), and H94 (R94T).

Here, as elsewhere, the first-mentioned residue is the residue of a humanized antibody formed by grafting Kabat CDRs or a composite Chothia-Kabat CDR in the case of CDR-H1 into a human acceptor framework, and the second-mentioned residue is a residue being considered for replacing such residue. Thus, within variable region frameworks, the first mentioned residue is human, and within CDRs, the first mentioned residue is mouse.

Exemplified antibodies include any permutations or combinations of the exemplified mature heavy and light chain variable regions hu12C4VHv1/hu12C4VLv1, hu12C4VHv1/hu12C4VLv2, hu12C4VHv2/hu12C4VLv1, hu12C4VHv2/hu12C4VLv2.

Exemplified antibodies include any permutations or combinations of the exemplified mature heavy chain variable regions hu12C4VHv1 (SEQ ID NO:221) and hu12C4VHv2 (SEQ ID NO:222), with any of the exemplified mature light chain variable regions hu12C4VLv1 (SEQ ID NO:223) and hu12C4VLv2 (SEQ ID NO:224).

The invention provides variants of the 12C4 humanized antibody in which the humanized mature heavy chain variable region shows at least 90%, 95%, 96%, 97%, 98%, or 99% identity to hu12C4VHv1 (SEQ ID NO:221) or hu12C4VHv2 (SEQ ID NO:222), and the humanized mature light chain variable region shows at least 90%, 95%, 96%, 97%, 98%, or 99% identity to hu12C4VLv1 (SEQ ID NO:223) or hu12C4VLv2 (SEQ ID NO:224). In some such antibodies at least 1, 2, 3, 4, 5, or all 6 of the backmutations or other mutations in SEQ ID NOs:221-222, and SEQ ID NOs:223-224) are retained.

In some humanized 12C4 antibodies, at least one of the following positions in the VH region is occupied by the amino acid as specified: H1 is occupied by Q or E, H48 is occupied by M or I, H93 is occupied by A or T, H94 is occupied by R or T.

In some humanized 12C4 antibodies, positions H1, H48, H93, and H94 in the VH region are occupied by E, I, T, and T, respectively, as in hu12C4VHv2.

In some humanized 12C4 antibodies, at least one of the following positions in the VL region is occupied by the amino acid as specified: L64 is G or S, L104 is V or L.

In some humanized 12C4 antibodies, positions L64 and L104 in the VL region are occupied by S and L, respectively, as in hu12C4VLv2.

In some humanized 12C4 antibodies, the variable heavy chain has ≥85% identity to human sequence. In some humanized 12C4 antibodies, the variable light chain has ≥85% identity to human sequence. In some humanized 12C4 antibodies, each of the variable heavy chain and variable light chain has ≥85% identity to human germline sequence. In some humanized 12C4 antibodies, the three heavy chain CDRs are as defined by Kabat/Chothia Composite (SEQ ID NOs:8, 220, and 10) and the three light chain CDRs are as defined by Kabat/Chothia Composite (SEQ ID NOs:12, 13, and 14).

Exemplary humanized antibodies are humanized forms of the mouse 17C12, designated Hu17C12.

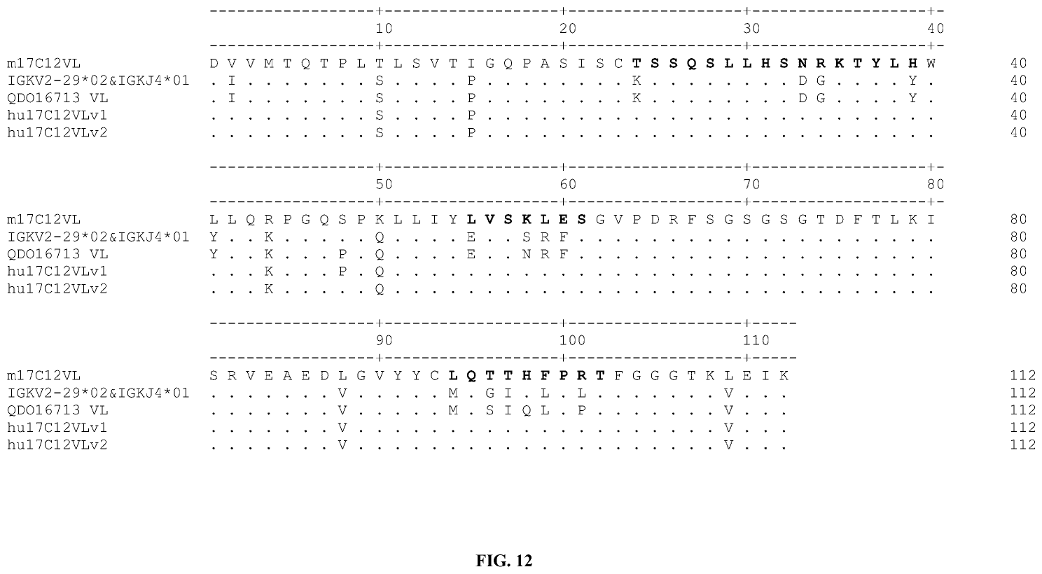

The mouse antibody 17C12 comprises mature heavy and light chain variable regions having amino acid sequences comprising SEQ ID NO:225 and SEQ ID NO:228, respectively. The invention provides 2 exemplified humanized mature heavy chain variable regions: hu17C12VHv and hu17C12VHv2. The invention further provides 2 exemplified mature light chain variable regions hu17C12VLv1 and hu17C12VLv2. FIGS. 11 and 12 show alignments of the heavy chain variable region and light chain variable region, respectively, of murine 17C12 and various humanized antibodies.

For reasons such as possible influence on CDR conformation and/or binding to antigen, mediating interaction between heavy and light chains, interaction with the constant region, being a site for desired or undesired post-translational modification, being an unusual residue for its position in a human variable region sequence and therefore potentially immunogenic, getting aggregation potential, and other reasons, the following 13 variable region framework positions were considered as candidates for substitutions in the 2 exemplified human mature light chain variable regions and the 2 exemplified human mature heavy chain variable regions, as further specified in the examples: L2 (I2V), L36 (Y36L), L43 (P43S), H1 (Q1E), H2 (V2I), H24 (V24A). H48 (M48I), H67 (V67A), H69 (I69M), H93 (A93T), H94 (R94T), H108 (T108L), and H113 (R113S).

Here, as elsewhere, the first-mentioned residue is the residue of a humanized antibody formed by grafting Kabat CDRs or a composite Chothia-Kabat CDR in the case of CDR-H1 into a human acceptor framework, and the second-mentioned residue is a residue being considered for replacing such residue. Thus, within variable region frameworks, the first mentioned residue is human, and within CDRs, the first mentioned residue is mouse.

Exemplified antibodies include any permutations or combinations of the exemplified mature heavy and light chain variable regions hu17C12VHv1/hu17C12VLv1, hu17C12VHv1/hu17C12VLv2, hu17C12VHv2/hu17C12VLv1, hu17C12VHv2/hu17C12VLv2.

Exemplified antibodies include any permutations or combinations of the exemplified mature heavy chain variable regions hu17C12VHv1 (SEQ ID NO:232) and hu17C12VHv2 (SEQ ID NO:233), with any of the exemplified mature light chain variable regions hu17C12VLv1 (SEQ ID NO:234) and hu17C12VLv2 (SEQ ID NO:235).

The invention provides variants of the 17C12 humanized antibody in which the humanized mature heavy chain variable region shows at least 90%, 95%, 96%, 97%, 98%, or 99% identity to hu17C12VHv1 (SEQ ID NO:232) or hu17C12VHv2 (SEQ ID NO:233), and the humanized mature light chain variable region shows at least 90%, 95%, 96%, 97%, 98%, or 99% identity to hu17C12VLv1 (SEQ ID NO:234) or hu17C12VLv2 (SEQ ID NO:235). In some such antibodies at least 1, 2, 3, 4, 5, 6, 7, 8, 9, 10, 11, 12, or all 13 of the backmutations or other mutations in SEQ ID NOs:232-233, and SEQ ID NOs:234-235 are retained.

In some humanized 17C12 antibodies, at least one of the following positions in the VH region is occupied by the amino acid as specified: H2 is occupied by I, H24 is occupied by A, H48 is occupied by I, H67 is occupied by A, H69 is occupied by M, H93 is occupied by T, and H94 is occupied by T. In some humanized 17C12 antibodies, positions H2, H24, H48, H67, H69, H93, and H94 are occupied by E, A, I, A, M, T, and T, respectively.

In some humanized 17C12 antibodies, at least one of the following positions in the VH region is occupied by the amino acid as specified: H1 is occupied by Q or E, H2 is occupied by I, H24 is occupied by A, H48 is occupied by I, H67 is occupied by A, H69 is occupied by M, H93 is occupied by T, H94 is occupied by T, H108 is occupied by T or L, H113 is occupied by R or S.

In some humanized 17C12 antibodies, positions H2, H24, H48, H67, H69, H93, and H94 in the VH region are occupied by E, A, I, A, M, T, and T , respectively, as in hu17C12VHv1. In some humanized 17C12 antibodies, positions H1, H2, H24, H48, H67, H69, H93, H94, H108, and H113 in the VH region are occupied by E, I, A, I, A, M, T. T. L, and S, respectively, as in hu17C12VHv2.

In some humanized 17C12 antibodies, at least one of the following positions in the VL region is occupied by the amino acid as specified: L2 is occupied by V, and L36 is occupied by L. In some humanized 17C12 antibodies, positions L2 and L36 are occupied by V and L, respectively.

In some humanized 17C12 antibodies, at least one of the following positions in the VL region is occupied by the amino acid as specified: L2 is V, L36 is L, L43 is P or S.

In some humanized 17C12 antibodies, positions L2 and L36 in the VL region are occupied by V and L, respectively, as in hu17C12VLv1. In some humanized 17C12 antibodies, positions L2, L36, and L43 in the VL region are occupied by V, L, and S, respectively, as in hu17C12VLv2.

In some humanized 17C12 antibodies, the variable heavy chain has ≥85% identity to human sequence. In some humanized 17C12 antibodies, the variable light chain has ≥85% identity to human sequence. In some humanized 17C12 antibodies, each of the variable heavy chain and variable light chain has ≥85% identity to human germline sequence. In some humanized 9F5 antibodies, the three heavy chain CDRs are as defined by Kabat/Chothia Composite (SEQ ID NOs:226, 227, and 10) and the three light chain CDRs are as defined by Kabat/Chothia Composite (SEQ ID NOs:229-231).

Exemplary humanized antibodies are humanized forms of the mouse 14H3, designated Hu14H3.

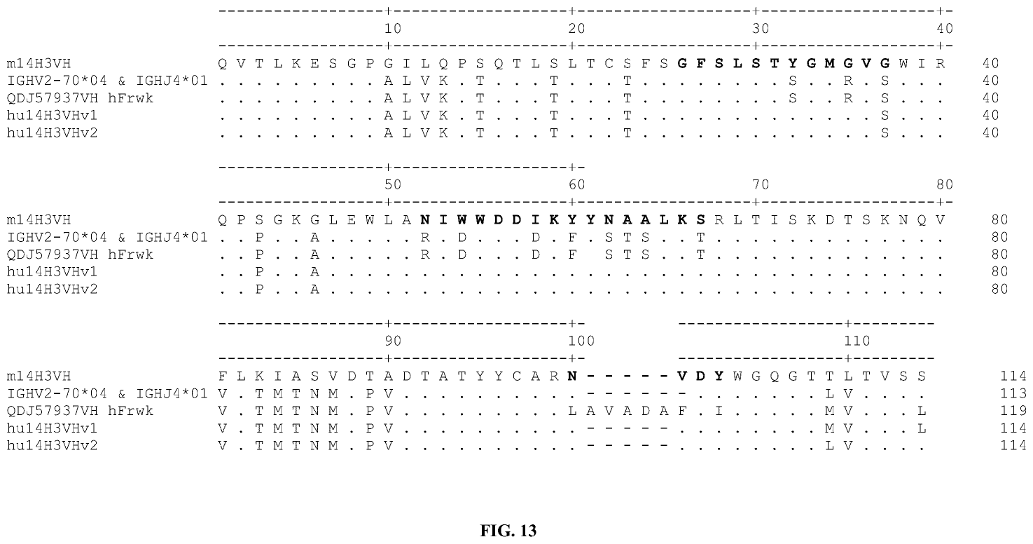

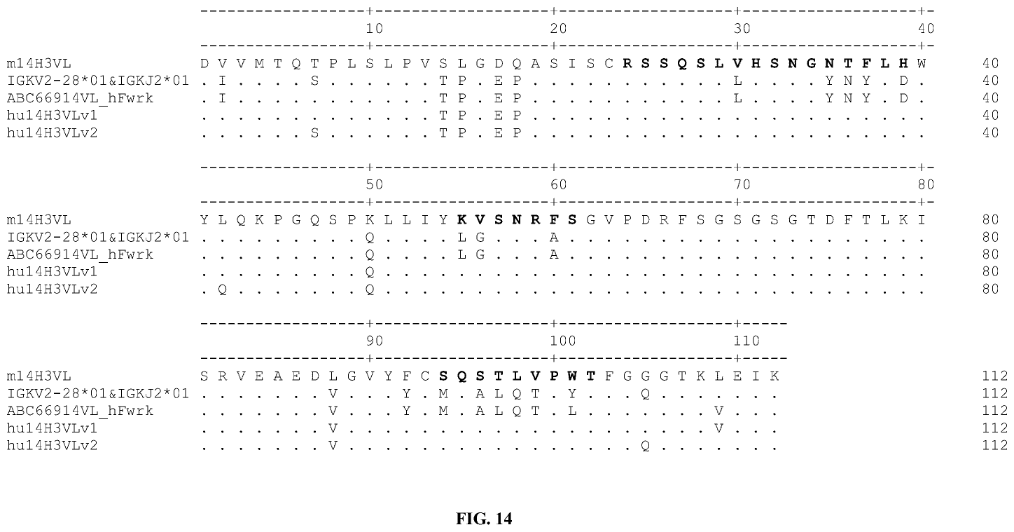

The mouse antibody 14H3 comprises mature heavy and light chain variable regions having amino acid sequences comprising SEQ ID NO:240 and SEQ ID NO:244, respectively. The invention provides 2 exemplified humanized mature heavy chain variable regions: hu14H3VHv1 and hu14H3VHv2. The invention further provides 2 exemplified mature light chain variable regions hu14H3VLv1 and hu14H3VLv2. FIGS. 13 and 14 show alignments of the heavy chain variable region and light chain variable region, respectively, of murine 14H3 and various humanized antibodies.

For reasons such as possible influence on CDR conformation and/or binding to antigen, mediating interaction between heavy and light chains, interaction with the constant region, being a site for desired or undesired post-translational modification, being an unusual residue for its position in a human variable region sequence and therefore potentially immunogenic, getting aggregation potential, and other reasons, the following 8 variable region framework positions were considered as candidates for substitutions in the 2 exemplified human mature light chain variable regions and the 2 exemplified human mature heavy chain variable regions, as further specified in the examples: L2 (I2V), L7 (T7S), L37 (L37Q), L87 (Y87F), L100 (G100Q), L104 (V104L), H108 (M108L), and H113 (L113S). The following variable region CDR position was considered as a candidate for substitutions in the 2 exemplified human mature heavy chain variable regions, as further specified in the examples: H35B (G35BS). In some humanized 14H3 antibodies, Kabat-Chothia Composite CDR-H1 has an amino acid sequence comprising SEQ ID NO:275.

Here, as elsewhere, the first-mentioned residue is the residue of a humanized antibody formed by grafting Kabat CDRs or a composite Chothia-Kabat CDR in the case of CDR-H1 into a human acceptor framework, and the second-mentioned residue is a residue being considered for replacing such residue. Thus, within variable region frameworks, the first mentioned residue is human, and within CDRs, the first mentioned residue is mouse.

Exemplified antibodies include any permutations or combinations of the exemplified mature heavy and light chain variable regions hu14H3VHv1/hu14H3VLv1, hu14H3VHv1/hu14H3VLv2, hu14H3VHv2/hu14H3VLv1, hu14H3VHv2/hu14H3VLv2.

Exemplified antibodies include any permutations or combinations of the exemplified mature heavy chain variable regions hu14H3VHv1 (SEQ ID NO:248) and hu14H3VHv2 (SEQ ID NO:249) with any of the exemplified mature light chain variable regions hu14H3VLv1 (SEQ ID NO:250) and hu14H3VLv2 (SEQ ID NO:251).

The invention provides variants of the 14H3 humanized antibody in which the humanized mature heavy chain variable region shows at least 90%, 95%, 96%, 97%, 98%, or 99% identity to hu14H3VHv1 (SEQ ID NO:249) or hu14H3VHv2 (SEQ ID NO:250) and the humanized mature light chain variable region shows at least 90%, 95%, 96%, 97%, 98%, or 99% identity to hu14H3VLv1 (SEQ ID NO:251) or hu14H3VLv2 (SEQ ID NO:252). In some such antibodies at least 1, 2, 3, 4, 5, 6, 7, 8, or all 9 of the backmutations or other mutations in SEQ ID NOs:249-250, and SEQ ID NOs:251-252 are retained.

In some humanized 14H3 antibodies, position H35B in the VH region is occupied by S.

In some humanized 14H3 antibodies, at least one of the following positions in the VH region is occupied by the amino acid as specified: H35B is occupied by S, H108 is occupied by M or L, H113 is occupied by L or S.

In some humanized 14H3 antibodies, position H35B in the VH region is occupied by S, as in hu14H3VHv1. In some humanized 14H3 antibodies, positions H35B, H108, and H113 in the VH region are occupied by S, L, and S, respectively, as in hu14H3VHv2.

In some humanized 14H3 antibodies, at least one of the following positions in the VL region is occupied by the amino acid as specified: L2 is occupied by V and L87 is occupied by F. In some humanized 14H3 antibodies, positions L2 and L87 are occupied by V and F, respectively.

In some humanized 14H3 antibodies, at least one of the following positions in the VL region is occupied by the amino acid as specified: L2 is V, L7 is T or S, L37 is L or Q, L87 is F, L100 is G or Q, L104 is V or L.

In some humanized 14H3 antibodies, positions L2 and L87 in the VL region are occupied by V and F, respectively, as in hu14H3VLv1. In some humanized 14H3 antibodies, positions L2, L7, L37, L87, L100, and L104 in the VL region are occupied by V, S, Q, F, Q, and L, respectively, as in hu14H3VLv2.

In some humanized 14H3 antibodies, the variable heavy chain has ≥85% identity to human sequence. In some humanized 14H3 antibodies, the variable light chain has ≥85% identity to human sequence. In some humanized 14H3 antibodies, each of the variable heavy chain and variable light chain has ≥85% identity to human germline sequence. In some humanized 14H3 antibodies, the three heavy chain CDRs are as defined by Kabat/Chothia Composite (SEQ ID NOs:241-243)); provided that position H35B is occupied by G or S and the three light chain CDRs are as defined by Kabat/Chothia Composite (SEQ ID NOs:245-247).

In some humanized 14H3 antibodies, Kabat-Chothia Composite CDR-H1 has an amino acid sequence comprising SEQ ID NO:275.

The CDR regions of such humanized 9F5, 10C12, 2D11, 12C4, 17C12, and 14H3 antibodies can be identical or substantially identical to the CDR regions of 9F5, 10C12, 2D11, 12C4, 17C12, or 14H3. The CDR regions can be defined by any conventional definition (e.g., Chothia, or composite of Chothia and Kabat) but are preferably as defined by Kabat.

Variable regions framework positions are in accordance with Kabat numbering unless otherwise stated. Other such variants typically differ from the sequences of the exemplified Hu9F5, Hu10C12, Hu12C4, Hu17C12, or Hu14H3 heavy and light chains by a small number (e.g., typically no more than 1, 2, 3, 5, 10, or 15) of replacements, deletions or insertions. Such differences are usually in the framework but can also occur in the CDRs.,

A possibility for additional variation in humanized 9F5, 10C12, 2D11, 12C4, 17C12, and 14H3 variants is additional backmutations in the variable region frameworks. Many of the framework residues not in contact with the CDRs in the humanized mAb can accommodate substitutions of amino acids from the corresponding positions of the donor mouse mAb or other mouse or human antibodies, and even many potential CDR-contact residues are also amenable to substitution. Even amino acids within the CDRs may be altered, for example, with residues found at the corresponding position of the human acceptor sequence used to supply variable region frameworks. In addition, alternate human acceptor sequences can be used, for example, for the heavy and/or light chain. If different acceptor sequences are used, one or more of the backmutations recommended above may not be performed because the corresponding donor and acceptor residues are already the same without backmutations.

Preferably, replacements or backmutations in humanized 9F5, 10C12, 2D11, 12C4, 17C12, and 14H3 variants (whether or not conservative) have no substantial effect on the binding affinity or potency of the humanized mAb, that is, its ability to bind to tau.

The humanized 9F5antibodies are further characterized by their ability to bind both phosphorylated and unphosphorylated tau and misfolded/aggregated forms of tau.

D. Chimeric and Veneered Antibodies

The invention further provides chimeric and veneered forms of non-human antibodies, particularly the 9F5, 10C12, 2D11, 12C4, 17C12, or 14H3 antibodies of the examples.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions. Veneered forms of the 9F5, 10C12, 2D11, 12C4, 17C12, and 14H3 antibodies are included in the invention.

E. Human Antibodies

Human antibodies specifically binding to tau or a fragment thereof (e.g., a peptide comprising or consisting of an amino acid sequence of QIVYKP (SEQ ID NO:57), EIVYKSP (SEQ ID NO:58), EIVYKS (SEQ ID NO:277), or (Q/E)IVYK(S/P) (SEQ ID NO:56)) are provided by a variety of techniques described below. Some human antibodies are selected by competitive binding experiments, by the phage display method of Winter, above, or otherwise, to have the same epitope specificity as a particular mouse antibody, such as one of the mouse monoclonal antibodies described in the examples. Human antibodies can also be screened for a particular epitope specificity by using only a fragment of tau, such as a tau fragment comprising or consisting of an amino acid sequence of QIVYKP (SEQ ID NO:57), EIVYKSP (SEQ ID NO:58), EIVYKS (SEQ ID NO:277), or (Q/E)IVYK(S/P) (SEQ ID NO:56) as the target antigen, and/or by screening antibodies against a collection of tau variants, such as tau variants containing various mutations within amino acid residues 307-312 or 391-397 or 391-396 of SEQ ID NO:1.

Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397; 5,874,299; 5,814,318; 5,789,650; 5,770,429; 5,661,016; 5,633,425; 5,625,126; 5,569,825; 5,545,806; Neuberger, Nat. Biotechnol. 14:826 (1996); and Kucherlapati, WO 91/10741 (1991)) phage display methods (see, e.g., Dower et al., WO 91/17271; McCafferty et al., WO 92/01047; U.S. Pat. Nos. 5,877,218; 5,871,907; 5,858,657; 5,837,242; 5,733,743; and 5,565,332); and methods described in WO 2008/081008 (e.g., immortalizing memory B cells isolated from humans, e.g., with EBV, screening for desired properties, and cloning and expressing recombinant forms).

F. Selection of Constant Region

The heavy and light chain variable regions of chimeric, veneered or humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Human IgG1 and IgG3 also induce stronger cell mediated effector functions than human IgG2 and IgG4. Light chain constant regions can be lambda or kappa. Numbering conventions for constant regions include EU numbering (Edelman, G. M. et al., Proc. Natl. Acad. USA, 63, 78-85 (1969)), Kabat numbering (Kabat, Sequences of Proteins of Immunological Interest (National Institutes of Health, Bethesda, MD, 1991, IMGT unique numbering (Lefranc M.-P. et al., IMGT unique numbering for immunoglobulin and T cell receptor constant domains and Ig superfamily C-like domains, Dev. Comp. Immunol., 29, 185-203 (2005), and IMGT exon numbering (Lefranc, supra).

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduce affinity for Fcγ receptors, particularly FcγRI receptor (see, e.g., U.S. Pat. No. 6,624,821). An alanine substitution at positions 234, 235, and 237 of human IgG1 can be used for reducing effector functions. Some antibodies have alanine substitution at positions 234, 235 and 237 of human IgG1 for reducing effector functions. Optionally, positions 234, 236 and/or 237 in human IgG2 are substituted with alanine and position 235 with glutamine (see, e.g., U.S. Pat. No. 5,624,821). In some antibodies, a mutation at one or more of positions 241, 264, 265, 270, 296, 297, 322, 329, and 331 by EU numbering of human IgG1 is used. In some antibodies, a mutation at one or more of positions 318, 320, and 322 by EU numbering of human IgG1 is used. In some antibodies, positions 234 and/or 235 are substituted with alanine and/or position 329 is substituted with glycine. In some antibodies, positions 234 and 235 are substituted with alanine. In some antibodies, the isotype is human IgG2 or IgG4.

Antibodies can be expressed as tetramers containing two light and two heavy chains, as separate heavy chains, light chains, as Fab, Fab′, F(ab′)2, and Fv, or as single chain antibodies in which heavy and light chain mature variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Thus, for example, another heavy chain constant region is of IgG1 G1m3with or without the C-terminal lysine. Reference to a human constant region includes a constant region with any natural allotype or any permutation of residues occupying positions in natural allotypes.

G. Expression of Recombinant Antibodies

A number of methods are known for producing chimeric and humanized antibodies using an antibody-expressing cell line (e.g., hybridoma). For example, the immunoglobulin variable regions of antibodies can be cloned and sequenced using well known methods. In one method, the heavy chain variable VH region is cloned by RT-PCR using mRNA prepared from hybridoma cells. Consensus primers are employed to the VH region leader peptide encompassing the translation initiation codon as the 5′ primer and a g2b constant regions specific 3′ primer. Exemplary primers are described in U.S. patent publication US 2005/0009150 by Schenk et al. (hereinafter “Schenk”). The sequences from multiple, independently derived clones can be compared to ensure no changes are introduced during amplification. The sequence of the VH region can also be determined or confirmed by sequencing a VH fragment obtained by 5′ RACE RT-PCR methodology and the 3′ g2b specific primer.

The light chain variable VL region can be cloned in an analogous manner. In one approach, a consensus primer set is designed for amplification of VL regions using a 5′ primer designed to hybridize to the VL region encompassing the translation initiation codon and a 3′ primer specific for the Ck region downstream of the V-J joining region. In a second approach, 5′RACE RT-PCR methodology is employed to clone a VL encoding cDNA. Exemplary primers are described in Schenk, supra. The cloned sequences are then combined with sequences encoding human (or other non-human species) constant regions.

In one approach, the heavy and light chain variable regions are re-engineered to encode splice donor sequences downstream of the respective VDJ or VJ junctions and are cloned into a mammalian expression vector, such as pCMV-hγ1 for the heavy chain and pCMV-Mcl for the light chain. These vectors encode human γ1 and Ck constant regions as exonic fragments downstream of the inserted variable region cassette. Following sequence verification, the heavy chain and light chain expression vectors can be co-transfected into CHO cells to produce chimeric antibodies. Conditioned media is collected 48 hours post-transfection and assayed by western blot analysis for antibody production or ELISA for antigen binding. The chimeric antibodies are humanized as described above.

Chimeric, veneered, humanized, and human antibodies are typically produced by recombinant expression. Recombinant polynucleotide constructs typically include an expression control sequence operably linked to the coding sequences of antibody chains, including naturally associated or heterologous expression control elements, such as a promoter. The expression control sequences can be promoter systems in vectors capable of transforming or transfecting eukaryotic or prokaryotic host cells. Once the vector has been incorporated into the appropriate host, the host is maintained under conditions suitable for high level expression of the nucleotide sequences and the collection and purification of the crossreacting antibodies.

These expression vectors are typically replicable in the host organisms either as episomes or as an integral part of the host chromosomal DNA. Commonly, expression vectors contain selection markers, e.g., ampicillin resistance or hygromycin resistance, to permit detection of those cells transformed with the desired DNA sequences.

E. coli is one prokaryotic host useful for expressing antibodies, particularly antibody fragments. Microbes, such as yeast, are also useful for expression. Saccharomyces is a yeast host with suitable vectors having expression control sequences, an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells can be used for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed, and include CHO cell lines, various COS cell lines, HeLa cells, HEK293 cells, L cells, and non-antibody-producing myelomas including Sp2/0 and NS0. The cells can be nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for light and/or heavy chains operably linked with a promoter and enhancer from a mammary gland specific gene, such as casein or beta lactoglobulin.

The vectors containing the DNA segments of interest can be transferred into the host cell by methods depending on the type of cellular host. For example, calcium chloride transfection is commonly utilized for prokaryotic cells, whereas calcium phosphate treatment, electroporation, lipofection, biolistics, or viral-based transfection can be used for other cellular hosts. Other methods used to transform mammalian cells include the use of polybrene, protoplast fusion, liposomes, electroporation, and microinjection. For production of transgenic animals, transgenes can be microinjected into fertilized oocytes or can be incorporated into the genome of embryonic stem cells or induced pluripotent stem cells (iPSCs), and the nuclei of such cells transferred into enucleated oocytes.

Having introduced vector(s) encoding antibody heavy and light chains into cell culture, cell pools can be screened for growth productivity and product quality in serum-free media. Top-producing cell pools can then be subjected of FACS-based single-cell cloning to generate monoclonal lines. Specific productivities above 50 pg or 100 pg per cell per day, which correspond to product titers of greater than 7.5 g/L culture, can be used. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, antibodies can be purified according to standard procedures of the art, including protein A capture, HPLC purification, column chromatography, gel electrophoresis and the like (see generally, Scopes, Protein Purification (Springer-Verlag, NY, 1982)).

Methodology for commercial production of antibodies can be employed, including codon optimization, selection of promoters, selection of transcription elements, selection of terminators, serum-free single cell cloning, cell banking, use of selection markers for amplification of copy number, CHO terminator, or improvement of protein titers (see, e.g., U.S. Pat. Nos. 5,786,464; 6,114,148; 6,063,598; 7,569,339; WO2004/050884; WO2008/012142; WO2008/012142; WO2005/019442; WO2008/107388; WO2009/027471; and U.S. Pat. No. 5,888,809).

### IV. Active Immunogens

An agent used for active immunization serves to induce in a patient the same types of antibody described in connection with passive immunization above. Agents used for active immunization can be the same types of immunogens used for generating monoclonal antibodies in laboratory animals, e.g., a peptide of 3-15 or 3-12 or 5-12, or 5-8 contiguous amino acids from a region of tau corresponding to residues 307-312 or 391-397 or 391-396 of SEQ ID NO:1, such as, for example, a tau peptide including or consisting of residues 307-312 or 391-397 or 391-396 of SEQ ID NO:1) or a tau peptide comprising or consisting of amino acid sequence QIVYKP (SEQ ID NO:57), a tau peptide comprising or consisting of amino acid sequence EIVYKSP (SEQ ID NO:58), a tau peptide comprising or consisting of amino acid sequence EIVYKS (SEQ ID NO:277), or a tau peptide comprising or consisting of amino acid sequence (Q/E)IVYK(S/P) (SEQ ID NO:56). For inducing antibodies binding to the same or overlapping epitope as 9F5, 10C12, 2D11, 12C4, 17C12, or 14H3, the epitope specificity of these antibodies can be mapped (e.g., by testing binding to a series of overlapping peptides spanning tau). A fragment of tau consisting of or including or overlapping the epitope can then be used as an immunogen. Such fragments are typically used in unphosphorylated form.

The heterologous carrier and adjuvant, if used may be the same as used for generating monoclonal antibody, but may also be selected for better pharmaceutical suitability for use in humans. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-S-glycerine cysteine (Pam₃Cys), mannan (a mannose polymer), or glucan (a β1→2 polymer)), cytokines (e.g., IL-1, IL-1 alpha and β peptides, IL-2, γ-INF, IL-10, GM-CSF), and chemokines (e.g., MIP1-α and β, and RANTES). Immunogens may be linked to the carriers with or without spacers amino acids (e.g., gly-gly). Additional carriers include virus-like particles. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLoS ONE 2(5):e415.) Alternatively, peptide immunogens can be linked to at least one artificial T-cell epitope capable of binding a large proportion of MHC Class II molecules., such as the pan DR epitope (“PADRE”). PADRE is described in U.S. Pat. No. 5,736,142, WO 95/07707, and Alexander J et al, Immunity, 1:751-761 (1994). Active immunogens can be presented in multimeric form in which multiple copies of an immunogen and/or its carrier are presented as a single covalent molecule.

Fragments are often administered with pharmaceutically acceptable adjuvants. The adjuvant increases the titer of induced antibodies and/or the binding affinity of induced antibodies relative to the situation if the peptide were used alone. A variety of adjuvants can be used in combination with an immunogenic fragment of tau to elicit an immune response. Some adjuvants augment the intrinsic response to an immunogen without causing conformational changes in the immunogen that affect the qualitative form of the response. Some adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, Mass.; now Antigenics, Inc., New York, N.Y.). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Another adjuvant is CpG (WO 98/40100). Adjuvants can be administered as a component of a therapeutic composition with an active agent or can be administered separately, before, concurrently with, or after administration of the therapeutic agent.

Analogs of natural fragments of tau that induce antibodies against tau can also be used. For example, one or more or all L-amino acids can be substituted with D amino acids in such peptides. Also the order of amino acids can be reversed (retro peptide). Optionally a peptide includes all D-amino acids in reverse order (retro-inverso peptide). Peptides and other compounds that do not necessarily have a significant amino acid sequence similarity with tau peptides but nevertheless serve as mimetics of tau peptides and induce a similar immune response. Anti-idiotypic antibodies against monoclonal antibodies to tau as described above can also be used. Such anti-Id antibodies mimic the antigen and generate an immune response to it (see Essential Immunology, Roit ed., Blackwell Scientific Publications, Palo Alto, Calif. 6th ed., p. 181).

Peptides (and optionally a carrier fused to the peptide) can also be administered in the form of a nucleic acid encoding the peptide and expressed in situ in a patient. A nucleic acid segment encoding an immunogen is typically linked to regulatory elements, such as a promoter and enhancer that allow expression of the DNA segment in the intended target cells of a patient. For expression in blood cells, as is desirable for induction of an immune response, promoter and enhancer elements from light or heavy chain immunoglobulin genes or the CMV major intermediate early promoter and enhancer are suitable to direct expression. The linked regulatory elements and coding sequences are often cloned into a vector. Antibodies can also be administered in the form of nucleic acids encoding the antibody heavy and/or light chains. If both heavy and light chains are present, the chains are preferably linked as a single chain antibody. Antibodies for passive administration can also be prepared e.g., by affinity chromatography from sera of patients treated with peptide immunogens.

The DNA can be delivered in naked form (i.e., without colloidal or encapsulating materials). Alternatively a number of viral vector systems can be used including retroviral systems (see, e.g., Lawrie and Tumin, Cur. Opin. Genet. Develop. 3, 102-109 (1993)) including retrovirus derived vectors such MMLV, HIV-1, and ALV; adenoviral vectors {see, e.g., Bett et al, J. Virol. 67, 591 1 (1993)); adeno-associated virus vectors {see, e.g., Zhou et al., J. Exp. Med. 179, 1867 (1994)), lentiviral vectors such as those based on HIV or FIV gag sequences, viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see U.S. Pat. No. 5,643,576) and rhabdoviruses, such as vesicular stomatitis virus (see WO 96/34625)and papillomaviruses (Ohe et al., Human Gene Therapy 6, 325-333 (1995); Woo et al, WO 94/12629 and Xiao & Brandsma, Nucleic Acids. Res. 24, 2630-2622 (1996)).

DNA encoding an immunogen, or encoding the antibody heavy and/or light chains, or a vector containing the same, can be packaged into liposomes. Suitable lipids and related analogs are described by U.S. Pat. Nos. 5,208,036, 5,264,618, 5,279,833, and 5,283,185. Vectors and DNA encoding an immunogen or encoding the antibody heavy and/or light chains can also be adsorbed to or associated with particulate carriers, examples of which include polymethyl methacrylate polymers and polylactides and poly(lactide-co-glycolides), (see, e.g., McGee et al., J. Micro Encap. 1996).

Vectors or segments therefrom encoding the antibody heavy and/or light chains can be incorporated in cells ex vivo, for example to cells explanted from an individual patient (e.g., lymphocytes, bone marrow aspirates, tissue biopsy) or universal donor hematopoietic stem cells, followed by reimplantation of the cells into a patient, usually after selection for cells which have incorporated the transgenes. (see, e.g., WO 2017/091512). Exemplary patient-derived cells include patient derived induced pluripotent stem cells (iPSCs) or other types of stem cells (embryonic, hematopoietic, neural, or mesenchymal).

A vector or segment therefrom encoding the antibody heavy and/or light chains can be introduced into any region of interest in cells ex vivo, such as an albumin gene or other safe harbor gene. Cells incorporating the vector can be implanted with or without prior differentiation. Cells can be implanted into a specific tissue, such as a secretory tissue or a location of pathology, or systemically, such as by infusion into the blood. For example, cells can be implanted into a secretory tissue of a patient, such as the liver, optionally with prior differentiation to cells present in that tissue, such as hepatocytes in the case of a liver. Expression of the antibody in the liver results in secretion of the antibody to the blood.

H. Antibody Screening Assays

Antibodies can be initially screened for the intended binding specificity as described above. Active immunogens can likewise be screened for capacity to induce antibodies with such binding specificity. In this case, an active immunogen is used to immunize a laboratory animal and the resulting sera tested for the appropriate binding specificity.

Antibodies having the desired binding specificity can then be tested in cellular and animal models. The cells used for such screening are preferentially neuronal cells. A cellular model of tau pathology has been reported in which neuroblastoma cells are transfected with a four-repeat domain of tau, optionally with a mutation associated with tau pathology (e.g., delta K280, see Khlistunova, Current Alzheimer Research 4, 544-546 (2007)). In another model, tau is induced in the neuroblastoma N2a cell line by the addition of doxycyclin. The cell models enable one to study the toxicity of tau to cells in the soluble or aggregated state, the appearance of tau aggregates after switching on tau gene expression, the dissolution of tau aggregates after switching the gene expression off again, and the efficiency of antibodies in inhibiting formation of tau aggregates or disaggregating them.

Antibodies or active immunogens can also be screened in transgenic animal models of diseases associated with tau. Such transgenic animals can include a tau transgene (e.g., any of the human isoforms) and optionally a human APP transgene among others, such as a kinase that phosphorylates tau, ApoE, presenilin or alpha synuclein. Such transgenic animals are disposed to develop at least one sign or symptom of a disease associated with tau.

An exemplary transgenic animal is the K3 line of mice (Itner et al., Proc. Natl. Acad. Sci. USA 105(41):15997-6002 (2008)). These mice have a human tau transgene with a K 369 I mutation (the mutation is associated with Pick's disease) and a Thy 1.2 promoter. This model shows a rapid course of neurodegeneration, motor deficit and degeneration of afferent fibers and cerebellar granule cells. Another exemplary animal is the JNPL3 line of mice. These mice have a human tau transgene with a P301L mutation (the mutation is associated with frontotemporal dementia) and a Thy 1.2 promoter (Taconic, Germantown, N.Y., Lewis, et al., Nat Genet. 25:402-405 (2000)). These mice have a more gradual course of neurodegeneration. The mice develop neurofibrillary tangles in several brain regions and spinal cord, which is hereby incorporated by reference in its entirety). This is an excellent model to study the consequences of tangle development and for screening therapy that may inhibit the generation of these aggregates. Another advantage of these animals is the relatively early onset of pathology. In the homozygous line, behavioral abnormalities associated with tau pathology can be observed at least as early as 3 months, but the animals remain relatively healthy at least until 8 months of age. In other words, at 8 months, the animals ambulate, feed themselves, and can perform the behavioral tasks sufficiently well to allow the treatment effect to be monitored. Active immunization of these mice for 6-13 months with—AI wI KLH-PHF-1 generated titers of about 1,000 and showed fewer neurofibrillary tangles, less pSer422, and reduced weight loss relative to untreated control ice.

The activity of antibodies or active agents can be assessed by various criteria including reduction in amount of total tau or phosphorylated tau, reduction in other pathological characteristics, such as amyloid deposits of Aβ, and inhibition or delay or behavioral deficits. Active immunogens can also be tested for induction of antibodies in the sera. Both passive and active immunogens can be tested for passage of antibodies across the blood brain barrier into the brain of a transgenic animal. Antibodies or fragments inducing an antibody can also be tested in non-human primates that naturally or through induction develop symptoms of diseases characterized by tau. Tests on an antibody or active agent are usually performed in conjunction with a control in which a parallel experiment is conduct except that the antibody or active agent is absent (e.g., replaced by vehicle). Reduction, delay or inhibition of signs or symptoms disease attributable to an antibody or active agent under test can then be assessed relative to the control.

### V. Patients Amenable to Treatment

The presence of neurofibrillary tangles has been found in several diseases including Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, and progressive supranuclear palsy (PSP). The present regimes can also be used in treatment or prophylaxis of any of these diseases. Because of the widespread association between neurological diseases and conditions and tau, the present regimes can be used in treatment or prophylaxis of any subject showing elevated levels of tau or phosphorylated tau (e.g., in the CSF) compared with a mean value in individuals without neurological disease. The present regimes can also be used in treatment or prophylaxis of neurological disease in individuals having a mutation in tau associated with neurological disease. The present methods are particularly suitable for treatment or prophylaxis of Alzheimer's disease, and especially in patients.

Patients amenable to treatment include individuals at risk of disease but not showing symptoms, as well as patients presently showing symptoms. Patients at risk of disease include those having a known genetic risk of disease. Such individuals include those having relatives who have experienced this disease, and those whose risk is determined by analysis of genetic or biochemical markers. Genetic markers of risk include mutations in tau, such as those discussed above, as well as mutations in other genes associated with neurological disease. For example, the ApoE4 allele in heterozygous and even more so in homozygous form is associated with risk of Alzheimer's disease. Other markers of risk of Alzheimer's disease include mutations in the APP gene, particularly mutations at position 717 and positions 670 and 671 referred to as the Hardy and Swedish mutations respectively, mutations in the presenilin genes, PS1 and PS2, a family history of AD, hypercholesterolemia or atherosclerosis. Individuals presently suffering from Alzheimer's disease can be recognized by PET imaging, from characteristic dementia, as well as the presence of risk factors described above. In addition, a number of diagnostic tests are available for identifying individuals who have AD. These include measurement of CSF tau or phospho-tau and Aβ42 levels. Elevated tau or phospho-tau and decreased Aβ42 levels signify the presence of AD. Some mutations associated with Parkinson's disease. Ala30Pro or Ala53, or mutations in other genes associated with Parkinson's disease such as leucine-rich repeat kinase, PARKS. Individuals can also be diagnosed with any of the neurological diseases mentioned above by the criteria of the DSM IV TR.

In asymptomatic patients, treatment can begin at any age (e.g., 10, 20, 30). Usually, however, it is not necessary to begin treatment until a patient reaches 40, 50, 60 or 70 years of age. Treatment typically entails multiple dosages over a period of time. Treatment can be monitored by assaying antibody levels over time. If the response falls, a booster dosage is indicated. In the case of potential Down's syndrome patients, treatment can begin antenatally by administering therapeutic agent to the mother or shortly after birth.

I. Nucleic Acids

The invention further provides nucleic acids encoding any of the heavy and light chains described above (e.g., SEQ ID NO:7, SEQ ID NO:11, SEQ ID NOs:15-22, SEQ ID NOs:23-29, 61-108, 109-129, 130-171, 214-217, 219, 221-224, 225, 228, 232-235, 240, 244, and 248-251). Optionally, such nucleic acids further encode a signal peptide and can be expressed with the signal peptide linked to the variable region. Coding sequences of nucleic acids can be operably linked with regulatory sequences to ensure expression of the coding sequences, such as a promoter, enhancer, ribosome binding site, transcription termination signal, and the like. The regulatory sequences can include a promoter, for example, a prokaryotic promoter or a eukaryotic promoter. The nucleic acids encoding heavy or light chains can be codon-optimized for expression in a host cell. The nucleic acids encoding heavy and light chains can encode a selectable gene. The nucleic acids encoding heavy and light chains can occur in isolated form or can be cloned into one or more vectors. The nucleic acids can be synthesized by, for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

J. Conjugated Antibodies

Conjugated antibodies that specifically bind to antigens, such as tau, are useful in detecting the presence of tau; monitoring and evaluating the efficacy of therapeutic agents being used to treat patients diagnosed with Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP); inhibiting or reducing aggregation of tau; inhibiting or reducing tau fibril formation; reducing or clearing tau deposits; stabilizing non-toxic conformations of tau; or treating or effecting prophylaxis of Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP) in a patient. For example, such antibodies can be conjugated with other therapeutic moieties, other proteins, other antibodies, and/or detectable labels. See WO 03/057838; U.S. Pat. No. 8,455,622. Such therapeutic moieties can be any agent that can be used to treat, combat, ameliorate, prevent, or improve an unwanted condition or disease in a patient, such as Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP).

Conjugated therapeutic moieties can include cytotoxic agents, cytostatic agents, neurotrophic agents, neuroprotective agents, radiotherapeutic agents, radioactive (radiopharmaceuticals), fluorescent, paramagnetic tracers, ultrasound contrast agents, immunomodulators, or any biologically active agents that facilitate or enhance the activity of the antibody, or modify bioavailability, and distribution in the body or within organs. A cytotoxic agent can be any agent that is toxic to a cell. A cytostatic agent can be any agent that inhibits cell proliferation. A neurotrophic agent can be any agent, including chemical or proteinaceous agents, that promotes neuron maintenance, growth, or differentiation. A neuroprotective agent can be agent, including chemical or proteinaceous agents, that protects neurons from acute insult or degenerative processes. An immunomodulator can be any agent that stimulates or inhibits the development or maintenance of an immunologic response. A radiotherapeutic agent can be any molecule or compound that emits radiation. If such therapeutic moieties are coupled to a tau-specific antibody, such as the antibodies described herein, the coupled therapeutic moieties will have a specific affinity for tau-related disease-affected cells over normal cells. Consequently, administration of the conjugated antibodies directly targets cancer cells with minimal damage to surrounding normal, healthy tissue. This can be particularly useful for therapeutic moieties that are too toxic to be administered on their own. In addition, smaller quantities of the therapeutic moieties can be used.

Some such antibodies can be modified to act as immunotoxins. See, e.g., U.S. Pat. No. 5,194,594. For example, ricin, a cellular toxin derived from plants, can be coupled to antibodies by using the bifunctional reagents S-acetylmercaptosuccinic anhydride for the antibody and succinimidyl 3-(2-pyridyldithio)propionate for ricin. See Pietersz et al., Cancer Res. 48(16):4469-4476 (1998). The coupling results in loss of B-chain binding activity of ricin, while impairing neither the toxic potential of the A-chain of ricin nor the activity of the antibody. Similarly, saporin, an inhibitor of ribosomal assembly, can be coupled to antibodies via a disulfide bond between chemically inserted sulfhydryl groups. See Polito et al., Leukemia 18:1215-1222 (2004).

Some such antibodies can be linked to radioisotopes. Examples of radioisotopes include, for example, yttrium⁹⁰ (90Y), indium¹¹¹ (111In), ¹³¹I, ⁹⁹mTc, radiosilver-111, radiosilver-199, and Bismuth²¹³. Linkage of radioisotopes to antibodies may be performed with conventional bifunction chelates. For radiosilver-111 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1:S91-S95 (2001).

Some such antibodies can be linked to other therapeutic moieties. Such therapeutic moieties can be, for example, cytotoxic, cytostatic, neurotrophic, or neuroprotective. For example, antibodies can be conjugated with toxic chemotherapeutic drugs such as maytansine, geldanamycin, tubulin inhibitors such as tubulin binding agents (e.g., auristatins), or minor groove binding agents such as calicheamicin. Other representative therapeutic moieties include agents known to be useful for treatment, management, or amelioration of Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP).

Antibodies can also be coupled with other proteins. For example, antibodies can be coupled with Fynomers. Fynomers are small binding proteins (e.g., 7 kDa) derived from the human Fyn SH3 domain. They can be stable and soluble, and they can lack cysteine residues and disulfide bonds. Fynomers can be engineered to bind to target molecules with the same affinity and specificity as antibodies. They are suitable for creating multi-specific fusion proteins based on antibodies. For example, Fynomers can be fused to N-terminal and/or C-terminal ends of antibodies to create bi- and tri-specific FynomAbs with different architectures. Fynomers can be selected using Fynomer libraries through screening technologies using FACS, Biacore, and cell-based assays that allow efficient selection of Fynomers with optimal properties. Examples of Fynomers are disclosed in Grabulovski et al., J. Biol. Chem. 282:3196-3204 (2007); Bertschinger et al., Protein Eng. Des. Sel. 20:57-68 (2007); Schlatter et al., MAbs. 4:497-508 (2011); Banner et al., Acta. Crystallogr. D. Biol. Crystallo 69(Pt6):1124-1137 (2013); and Brack et al., Mol. Cancer Ther. 13:2030-2039 (2014).

The antibodies disclosed herein can also be coupled or conjugated to one or more other antibodies (e.g., to form antibody heteroconjugates). Such other antibodies can bind to different epitopes within tau or can bind to a different target antigen.

Antibodies can also be coupled with a detectable label. Such antibodies can be used, for example, for diagnosing Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP), and/or for assessing efficacy of treatment. Such antibodies are particularly useful for performing such determinations in subjects having or being susceptible to Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP), or in appropriate biological samples obtained from such subjects. Representative detectable labels that may be coupled or linked to an antibody include various enzymes, such as horseradish peroxidase, alkaline phosphatase, beta-galactosidase, or acetylcholinesterase; prosthetic groups, such streptavidin/biotin and avidin/biotin; fluorescent materials, such as umbelliferone, fluorescein, fluorescein isothiocyanate, rhodamine, dichlorotriazinylamine fluorescein, dansyl chloride or phycoerythrin; luminescent materials, such as luminol; bioluminescent materials, such as luciferase, luciferin, and aequorin; radioactive materials, such as radiosilver-111, radiosilver-199, Bismuth²¹³, iodine (¹³¹I, ¹²⁵I, ¹²³I, ¹²¹I) carbon (¹⁴C), sulfur (⁵S), tritium (³H), indium (¹¹⁵In, ¹¹³In, ¹¹²In, ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies; nonradioactive paramagnetic metal ions; and molecules that are radiolabeled or conjugated to specific radioisotopes.

Linkage of radioisotopes to antibodies may be performed with conventional bifunction chelates. For radiosilver-111 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1:S91-S95 (2001).

Therapeutic moieties, other proteins, other antibodies, and/or detectable labels may be coupled or conjugated, directly or indirectly through an intermediate (e.g., a linker), to an antibody of the invention. See e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985); and Thorpe et al., Immunol. Rev., 62:119-58 (1982). Suitable linkers include, for example, cleavable and non-cleavable linkers. Different linkers that release the coupled therapeutic moieties, proteins, antibodies, and/or detectable labels under acidic or reducing conditions, on exposure to specific proteases, or under other defined conditions can be employed.

### VI. Pharmaceutical Compositions and Methods of Use

In prophylactic applications, an antibody or agent for inducing an antibody or a pharmaceutical composition the same is administered to a patient susceptible to, or otherwise at risk of a disease (e.g., Alzheimer's disease) in regime (dose, frequency and route of administration) effective to reduce the risk, lessen the severity, or delay the onset of at least one sign or symptom of the disease. In particular, the regime is preferably effective to inhibit or delay tau or phospho-tau and paired filaments formed from it in the brain, and/or inhibit or delay its toxic effects and/or inhibit/or delay development of behavioral deficits. In therapeutic applications, an antibody or agent to induce an antibody is administered to a patient suspected of, or already suffering from a disease (e.g., Alzheimer's disease) in a regime (dose, frequency and route of administration) effective to ameliorate or at least inhibit further deterioration of at least one sign or symptom of the disease. In particular, the regime is preferably effective to reduce or at least inhibit further increase of levels of tau, phosphor-tau, or paired filaments formed from it , associated toxicities and/or behavioral deficits.

A regime is considered therapeutically or prophylactically effective if an individual treated patient achieves an outcome more favorable than the mean outcome in a control population of comparable patients not treated by methods of the invention, or if a more favorable outcome is demonstrated in treated patients versus control patients in a controlled clinical trial (e.g., a phase II, phase II/III or phase III trial) at the p<0.05 or 0.01 or even 0.001 level.

Effective doses of vary depending on many different factors, such as means of administration, target site, physiological state of the patient, whether the patient is an ApoE carrier, whether the patient is human or an animal, other medications administered, and whether treatment is prophylactic or therapeutic.

Exemplary dosage ranges for antibodies are from about 0.01 to 60 mg/kg, or from about 0.1 to 3 mg/kg or 0.15-2 mg/kg or 0.15-1.5 mg/kg, of patient body weight. Antibody can be administered such doses daily, on alternative days, weekly, fortnightly, monthly, quarterly, or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months.

The amount of an agent for active administration varies from 0.1-500 μg per patient and more usually from 1-100 or 1-10 μg per injection for human administration. The timing of injections can vary significantly from once a day, to once a year, to once a decade. A typical regimen consists of an immunization followed by booster injections at time intervals, such as 6 week intervals or two months. Another regimen consists of an immunization followed by booster injections 1, 2 and 12 months later. Another regimen entails an injection every two months for life. Alternatively, booster injections can be on an irregular basis as indicated by monitoring of immune response.

Antibodies or agents for inducing antibodies are preferably administered via a peripheral route (i.e., one in which an administered or induced antibody crosses the blood brain barrier to reach an intended site in the brain. Routes of administration include topical, intravenous, oral, subcutaneous, intraarterial, intracranial, intrathecal, intraperitoneal, intranasal, intraocular, intradermal, or intramuscular. Some routes for administration of antibodies are intravenous and subcutaneous. Some routes for active immunization are subcutaneous and intramuscular. This type of injection is most typically performed in the arm or leg muscles. In some methods, agents are injected directly into a particular tissue where deposits have accumulated, for example intracranial injection.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

The present regimes can be administered in combination with another agent effective in treatment or prophylaxis of the disease being treated. For example, in the case of Alzheimer's disease, the present regimes can be combined with immunotherapy against Aβ (WO/2000/072880), cholinesterase inhibitors or memantine or in the case of Parkinson's disease immunotherapy against alpha synuclein WO/2008/103472, Levodopa, dopamine agonists, COMT inhibitors, MAO-B inhibitors, Amantadine, or anticholinergic agents.

Antibodies are administered in an effective regime meaning a dosage, route of administration and frequency of administration that delays the onset, reduces the severity, inhibits further deterioration, and/or ameliorates at least one sign or symptom of a disorder being treated. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

Exemplary dosages for an antibody are 0.1-60 mg/kg (e.g., 0.5, 3, 10, 30, or 60 mg/kg), or 0.5-5 mg/kg body weight (e.g., 0.5, 1, 2, 3, 4 or 5 mg/kg) or 10-4000 mg or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Some antibodies can be administered into the systemic circulation by intravenous or subcutaneous administration. Intravenous administration can be, for example, by infusion over a period such as 30-90 min.

The frequency of administration depends on the half-life of the antibody in the circulation, the condition of the patient and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

A. Diagnostics and Monitoring Methods

### In Vivo Imaging, Diagnostic Methods, and Optimizing Immunotherapy

The invention provides methods of in vivo imaging tau protein deposits (e.g., neurofibrillary tangles and tau inclusions) in a patient. The methods work by administering a reagent, such as antibody that binds tau (e.g., a mouse, humanized, chimeric or veneered 9F5, 10C12, 2D11, 12C4, 17C12, or 14H3 antibody), to the patient and then detecting the agent after it has bound. Antibodies specifically binding to tau at an epitope within amino acid residues QIVYKP (SEQ ID NO:57), EIVYKSP (SEQ ID NO:58), EIVYKS (SEQ ID NO:277), or (Q/E)IVYK(S/P) (SEQ ID NO:56) can be used. In some methods, the antibody binds to a peptide consisting of an epitope within amino acid residues QIVYKP (SEQ ID NO:57), EIVYKSP (SEQ ID NO:58), EIVYKS (SEQ ID NO:277), or (Q/E)IVYK(S/P) (SEQ ID NO:56). A clearing response to the administered antibodies can be avoided or reduced by using antibody fragments lacking a full-length constant region, such as Fabs. In some methods, the same antibody can serve as both a treatment and diagnostic reagent.

Diagnostic reagents can be administered by intravenous injection into the body of the patient, or directly into the brain by intracranial injection or by drilling a hole through the skull. The dosage of reagent should be within the same ranges as for treatment methods. Typically, the reagent is labeled, although in some methods, the primary reagent with affinity for tau is unlabeled and a secondary labeling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using positron emission tomography (PET) or single-photon emission computed tomography (SPECT).

The methods of in vivo imaging of tau protein deposits are useful to diagnose or confirm diagnosis of a tauopathy, such as Alzheimer's disease, frontotemporal lobar degeneration, progressive supranuclear palsy and Pick's disease, or susceptibility to such a disease. For example, the methods can be used on a patient presenting with symptoms of dementia. If the patient has abnormal neurofibrillary tangles, then the patient is likely suffering from Alzheimer's disease. Alternatively, if the patient has abnormal tau inclusions, then depending on the location of the inclusions, the patient may be suffering from frontotemporal lobar degeneration. The methods can also be used on asymptomatic patients. Presence of abnormal tau protein deposits indicates susceptibility to future symptomatic disease. The methods are also useful for monitoring disease progression and/or response to treatment in patients who have been previously diagnosed with a tau-related disease.

Diagnosis can be performed by comparing the number, size, and/or intensity of labeled loci, to corresponding baseline values. The base line values can represent the mean levels in a population of undiseased individuals. Baseline values can also represent previous levels determined in the same patient. For example, baseline values can be determined in a patient before beginning tau immunotherapy treatment, and measured values thereafter compared with the baseline values. A decrease in values relative to baseline signals a positive response to treatment.

In some patients, diagnosis of a tauopathy may be aided by performing a PET scan. A PET scan can be performed using, for example, a conventional PET imager and auxiliary equipment. The scan typically includes one or more regions of the brain known in general to be associated with tau protein deposits and one or more regions in which few if any deposits are generally present to serve as controls.

The signal detected in a PET scan can be represented as a multidimensional image. The multidimensional image can be in two dimensions representing a cross-section through the brain, in three dimensions, representing the three dimensional brain, or in four dimensions representing changes in the three dimensional brain over time. A color scale can be used with different colors indicating different amounts of label and, inferentially, tau protein deposit detected. The results of the scan can also be presented numerically, with numbers relating to the amount of label detected and consequently amount of tau protein deposits. The label present in a region of the brain known to be associated with deposits for a particular tauopathy (e.g., Alzheimer's disease) can be compared with the label present in a region known not to be associated with deposits to provide a ratio indicative of the extent of deposits within the former region. For the same radiolabeled ligand, such ratios provide a comparable measure of tau protein deposits and changes thereof between different patients.

In some methods, a PET scan is performed concurrent with or in the same patient visit as an Mill or CAT scan. An MM or CAT scan provides more anatomical detail of the brain than a PET scan. However, the image from a PET scan can be superimposed on an Mill or CAT scan image more precisely indicating the location of PET ligand and inferentially tau deposits relative to anatomical structures in the brain. Some machines can perform both PET scanning and Mill or CAT scanning without the patient changing positions between the scans facilitating superimposition of images.

Suitable PET ligands include radiolabeled antibodies of the invention (e.g., a mouse, humanized, chimeric or veneered 9F5, 10C12, 2D11, 12C4, 17C12, or 14H3 antibody). The radioisotope used can be, for example, C¹¹, N¹³, O¹⁵, F¹⁸, or I¹²³. The interval between administering the PET ligand and performing the scan can depend on the PET ligand and particularly its rate of uptake and clearing into the brain, and the half-life of its radiolabel.

PET scans can also be performed as a prophylactic measure in asymptomatic patients or in patients who have symptoms of mild cognitive impairment but have not yet been diagnosed with a tauopathy but are at elevated risk of developing a tauopathy. For asymptomatic patients, scans are particularly useful for individuals considered at elevated risk of tauopathy because of a family history, genetic or biochemical risk factors, or mature age. Prophylactic scans can commence for example, at a patient age between 45 and 75 years. In some patients, a first scan is performed at age 50 years.

Prophylactic scans can be performed at intervals of for example, between six months and ten years, preferably between 1-5 years. In some patients, prophylactic scans are performed annually. If a PET scan performed as a prophylactic measure indicates abnormally high levels of tau protein deposits, immunotherapy can be commenced and subsequent PET scans performed as in patients diagnosed with a tauopathy. If a PET scanned performed as a prophylactic measure indicates levels of tau protein deposits within normal levels, further PET scans can performed at intervals of between six months and 10 years, and preferably 1-5 years, as before, or in response to appearance of signs and symptoms of a tauopathy or mild cognitive impairment. By combining prophylactic scans with administration of tau-directed immunotherapy if and when an above normal level of tau protein deposits is detected, levels of tau protein deposits can be reduced to, or closer to, normal levels, or at least inhibited from increasing further, and the patient can remain free of the tauopathy for a longer period than if not receiving prophylactic scans and tau-directed immunotherapy (e.g., at least 5, 10, 15 or 20 years, or for the rest of the patient's life).

Normal levels of tau protein deposits can be determined by the amount of neurofibrillary tangles or tau inclusions in the brains of a representative sample of individuals in the general population who have not been diagnosed with a particular tauopathy (e.g., Alzheimer's disease) and are not considered at elevated risk of developing such disease (e.g., a representative sample of disease-free individuals under 50 years of age). Alternatively, a normal level can be recognized in an individual patient if the PET signal according to the present methods in a region of the brain in which tau protein deposits are known to develop is not different (within the accuracy of measurement) from the signal from a region of the brain in which it is known that such deposits do not normally develop. An elevated level in an individual can be recognized by comparison to the normal levels (e.g., outside mean and variance of a standard deviation) or simply from an elevated signal beyond experimental error in a region of the brain associated with tau protein deposits compared with a region not known to be associated with deposits. For purposes of comparing the levels of tau protein deposits in an individual and population, the tau protein deposits should preferably be determined in the same region(s) of the brain, these regions including at least one region in which tau protein deposits associated with a particular tauopathy (e.g., Alzheimer's disease) are known to form. A patient having an elevated level of tau protein deposits is a candidate for commencing immunotherapy.

After commencing immunotherapy, a decrease in the level of tau protein deposits can be first seen as an indication that the treatment is having the desired effect. The observed decrease can be, for example, in the range of 1-100%, 1-50%, or 1-25% of the baseline value. Such effects can be measured in one or more regions of the brain in which deposits are known to form or can be measured from an average of such regions. The total effect of treatment can be approximated by adding the percentage reduction relative to baseline to the increase in tau protein deposits that would otherwise occur in an average untreated patient.

Maintenance of tau protein deposits at an approximately constant level or even a small increase in tau protein deposits can also be an indication of response to treatment albeit a suboptimal response. Such responses can be compared with a time course of levels of tau protein deposits in patients with a particular tauopathy (e.g., Alzheimer's disease) that did not receive treatment, to determine whether the immunotherapy is having an effect in inhibiting further increases of tau protein deposits.

Monitoring of changes in tau protein deposits allows adjustment of the immunotherapy or other treatment regime in response to the treatment. PET monitoring provides an indication of the nature and extent of response to treatment. Then a determination can be made whether to adjust treatment and if desired treatment can be adjusted in response to the PET monitoring. PET monitoring thus allows for tau-directed immunotherapy or other treatment regime to be adjusted before other biomarkers, MRI or cognitive measures have detectably responded. A significant change means that comparison of the value of a parameter after treatment relative to basement provides some evidence that treatment has or has not resulted in a beneficial effect. In some instances, a change of values of a parameter in a patient itself provides evidence that treatment has or has not resulted in a beneficial effect. In other instances, the change of values, if any, in a patient, is compared with the change of values, if any, in a representative control population of patients not undergoing immunotherapy. A difference in response in a particular patient from the normal response in the control patient (e.g., mean plus variance of a standard deviation) can also provide evidence that an immunotherapy regime is or is not achieving a beneficial effect in a patient.

In some patients, monitoring indicates a detectable decline in tau protein deposits but that the level of tau protein deposits remains above normal. In such patients, if there are no unacceptable side effects, the treatment regime can be continued as is or even increased in frequency of administration and/or dose if not already at the maximum recommended dose.

If the monitoring indicates levels of tau protein deposits in a patient have already been reduced to normal, or near-normal, levels of tau protein deposits, the immunotherapy regime can be adjusted from one of induction (i.e., that reduces the level of tau protein deposits) to one of maintenance (i.e. , that maintains tau protein deposits at an approximately constant level). Such a regime can be affected by reducing the dose and or frequency of administering immunotherapy.

In other patients, monitoring can indicate that immunotherapy is having some beneficial effect but a suboptimal effect. An optimal effect can be defined as a percentage reduction in the level of tau protein deposits within the top half or quartile of the change in tau protein deposits (measured or calculated over the whole brain or representative region(s) thereof in which tau protein deposits are known to form) experienced by a representative sample of tauopathy patients undergoing immunotherapy at a given time point after commencing therapy. A patient experiencing a smaller decline or a patient whose tau protein deposits remains constant or even increases, but to a lesser extent than expected in the absence of immunotherapy (e.g., as inferred from a control group of patients not administered immunotherapy) can be classified as experiencing a positive but suboptimal response. Such patients can optionally be subject to an adjustment of regime in which the dose and or frequency of administration of an agent is increased.

In some patients, tau protein deposits may increase in similar or greater fashion to tau deposits in patients not receiving immunotherapy. If such increases persist over a period of time, such as 18 months or 2 years, even after any increase in the frequency or dose of agents, immunotherapy can if desired be discontinued in favor of other treatments.

The foregoing description of diagnosing, monitoring, and adjusting treatment for tauopathies has been largely focused on using PET scans. However, any other technique for visualizing and/or measuring tau protein deposits that is amenable to the use of tau antibodies of the invention (e.g., a mouse, humanized, chimeric or veneered 9F5, 10C12, 2D11, 12C4, 17C12, or 14H3 antibody) can be used in place of PET scans to perform such methods.

Also provided are methods of detecting an immune response against tau in a patient suffering from or susceptible to diseases associated with tau. The methods can be used to monitor a course of therapeutic and prophylactic treatment with the agents provided herein. The antibody profile following passive immunization typically shows an immediate peak in antibody concentration followed by an exponential decay. Without a further dose, the decay approaches pretreatment levels within a period of days to months depending on the half-life of the antibody administered. For example, the half-life of some human antibodies is of the order of 20 days.

In some methods, a baseline measurement of antibody to tau in the subject is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dose of antibody is administered. In some methods, peak or subsequent measured levels less background are compared with reference levels previously determined to constitute a beneficial prophylactic or therapeutic treatment regime in other subjects. If the measured antibody level is significantly less than a reference level (e.g., less than the mean minus one or, preferably, two standard deviations of the reference value in a population of subjects benefiting from treatment) administration of an additional dose of antibody is indicated.

Also provided are methods of detecting tau in a subject, for example, by measuring tau in a sample from a subject or by in vivo imaging of tau in a subject. Such methods are useful to diagnose or confirm diagnosis of diseases associated with tau, or susceptibility thereto. The methods can also be used on asymptomatic subjects. The presence of tau indicates susceptibility to future symptomatic disease. The methods are also useful for monitoring disease progression and/or response to treatment in subjects who have been previously diagnosed with Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP).

Biological samples obtained from a subject having, suspected of having, or at risk of having Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP) can be contacted with the antibodies disclosed herein to assess the presence of tau. For example, levels of tau in such subjects may be compared to those present in healthy subjects. Alternatively, levels of tau in such subjects receiving treatment for the disease may be compared to those of subjects who have not been treated for Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP). Some such tests involve a biopsy of tissue obtained from such subjects. ELISA assays may also be useful methods, for example, for assessing tau in fluid samples.

### VII. Kits

The invention further provides kits (e.g., containers) comprising an antibody disclosed herein and related materials, such as instructions for use (e.g., package insert). The instructions for use may contain, for example, instructions for administration of the antibody and optionally one or more additional agents. The containers of antibody may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products

Kits can also include a second container comprising a pharmaceutically-acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

### VIII. Other Applications

The antibodies can be used for detecting tau, or fragments thereof, in the context of clinical diagnosis or treatment or in research. For example, the antibodies can be used to detect the presence of tau in a biological sample as an indication that the biological sample comprises tau deposits. Binding of the antibodies to the biological sample can be compared to binding of the antibodies to a control sample. The control sample and the biological sample can comprise cells of the same tissue origin. Control samples and biological samples can be obtained from the same individual or different individuals and on the same occasion or on different occasions. If desired, multiple biological samples and multiple control samples are evaluated on multiple occasions to protect against random variation independent of the differences between the samples. A direct comparison can then be made between the biological sample(s) and the control sample(s) to determine whether antibody binding (i.e., the presence of tau) to the biological sample(s) is increased, decreased, or the same relative to antibody binding to the control sample(s). Increased binding of the antibody to the biological sample(s) relative to the control sample(s) indicates the presence of tau in the biological sample(s). In some instances, the increased antibody binding is statistically significant. Optionally, antibody binding to the biological sample is at least 1.5-fold, 2-fold, 3-fold, 4-fold, 5-fold, 10-fold, 20-fold, or 100-fold higher than antibody binding to the control sample.

In addition, the antibodies can be used to detect the presence of the tau in a biological sample to monitor and evaluate the efficacy of a therapeutic agent being used to treat a patient diagnosed with Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP). A biological sample from a patient diagnosed with Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP) is evaluated to establish a baseline for the binding of the antibodies to the sample (i.e., a baseline for the presence of the tau in the sample) before commencing therapy with the therapeutic agent. In some instances, multiple biological samples from the patient are evaluated on multiple occasions to establish both a baseline and measure of random variation independent of treatment. A therapeutic agent is then administered in a regime. The regime may include multiple administrations of the agent over a period of time. Optionally, binding of the antibodies (i.e., presence of tau) is evaluated on multiple occasions in multiple biological samples from the patient, both to establish a measure of random variation and to show a trend in response to immunotherapy. The various assessments of antibody binding to the biological samples are then compared. If only two assessments are made, a direct comparison can be made between the two assessments to determine whether antibody binding (i.e., presence of tau) has increased, decreased, or remained the same between the two assessments. If more than two measurements are made, the measurements can be analyzed as a time course starting before treatment with the therapeutic agent and proceeding through the course of therapy. In patients for whom antibody binding to biological samples has decreased (i.e., the presence of tau), it can be concluded that the therapeutic agent was effective in treating the Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP) in the patient. The decrease in antibody binding can be statistically significant. Optionally, binding decreases by at least 1%, 2%, 3%, 4%, 5%, 10%, 15%, 20%, 30%, 40%, 50%, 60%, 70%, 80%, 90%, or 100%. Assessment of antibody binding can be made in conjunction with assessing other signs and symptoms of Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP).

The antibodies can also be used as research reagents for laboratory research in detecting tau, or fragments thereof. In such uses, antibodies can be labeled with fluorescent molecules, spin-labeled molecules, enzymes, or radioisotopes, and can be provided in the form of kit with all the necessary reagents to perform the detection assay. The antibodies can also be used to purify tau, or binding partners of tau, e.g., by affinity chromatography.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1. Identification and Screening of Tau Monoclonal Antibodies

Immunizations were performed with either recombinant N-terminally His-tagged 383 a.a. human tau (4R0N), containing a P301S mutation [immunogen A] or recombinant 383 a.a. human tau (4R0N), containing a P301S mutation, lacking an N-terminal His-tag [immunogen B]. Immunogens were emulsified in RIM adjuvant.

Five week old female A/J mice were intraperitoneally immunized with 25 μg of immunogen A on day 0, and 10 μg of immunogen A each on days 7, 14, 21, 27, 34, 48, and 55. For antibody 10C12, on day 43 mice were bled and titered against immunogen A. The animals with highest titers were boosted on day 54 with a terminal immunization of 50 μg immunogen A, which was delivered ½ intraperitoneally and ½ intravenously.

For antibodies 9F5, 17C12, 2D11, 14H3, and 12C4 the animals were immunized with an additional 10 μg of immunogen A on day 62 and 10 μg of immunogen B on days 76 and 90. On days 43 and 98, mice were bled and titered against immunogen A. Animals with highest titers were boosted on day 101 with a terminal immunization of 50 μg immunogen B, which was delivered ½ intraperitoneally and ½ intravenously. For all antibodies, fused hybridomas were screened via ELISA against both immunogens.

An internalization assay employing fluorescence activated cell sorting (FACS) was performed to evaluate the ability of various antibodies to block neuronal internalization of tau. Antibodies that block internalization will likely block transmission of tau. pHrodo-labeled tau P301L soluble oligomer (1.5 μg/ml final concentration) was preincubated with anti-tau antibodies (dose titration: 80 μg/ml starting concentration followed by 4-fold serial dilutions) for 30 min at room temperature in cell culture media. TAU/antibody mixture was then added to B103 neuroblastoma cell lines at 500,000 cells/ml final concentration and incubated for 3-4 hrs at 37° C. in tissue culture incubator (5% CO₂). Cells were then washed 3× with culture media, followed by 10 minutes culture media incubation, and washed 2× with FACS buffer (1% FBS in PBS). Cells were resuspended in 100 μl FACS buffer and Texas Red mean fluorescence intensity measured by FACS LSR II. Texas red fluorescence from pHrodo is activated by low pH associated with endolysosomal compartments upon internalization. Because FACS detects cells and pHrodo only fluoresces upon internalization, only tau internalized by the cells will be detected. The lower the mean fluorescence intensity, the lower the amount of internalized tau, which suggests a higher blocking activity of the antibody tested. As shown in Table 55 and FIG. 3, mouse 3D6 (WO 2017/191560) and mouse 9F5 antibody block neuronal internalization significantly more than the other antibodies tested. Mouse 9F5 was selected based on its novel epitope and the results in the pHrodo assay.

TABLE 55
Internalization assay results with mouse antibodies 9F5, 3D6, 5G8, 16G7, and 16B5
All values are % of maximum signal compared to cells with no treatment

| Antibody | | | | | | |
|---|---|---|---|---|---|---|
| concentration | | | | | | |
| (μg/mL) | Control | 3D6 | 9F5 | 5G8 | 16G7 | 16B5 |
| 62.5 | 87.7 ± 4.8 | 14.2 ± 1.7 | 18.1 ± 5 | 77.7 ± 3.2 | 33.9 ± 1.3 | 61.9 ± 5.7 |
| 15.625 | 86.2 ± 2.6 | 31 ± 1.6 | 24.2 ± 3.9 | 88.1 ± 7.6 | 51.4 ± 0.8 | 72.8 ± 6.2 |
| 3.9 | 81.2 ± 5.3 | 41.2 ± 2.2 | 35.9 ± 4.2 | 94.3 ± 5.3 | 62.2 ± 6.5 | 80.1 ± 3.1 |
| 0.98 | 75.4 ± 4.8 | 64.3 ± 0.6 | 71 ± 6.0 | 78.8 ± 3.7 | 56 ± 4.3 | 88.1 ± 9.1 |
| 0.244 | 82.9 ± 1.5 | 82.2 ± 9.2 | 71.7 ± 6.5 | 86 ± 4.6 | 74.6 ± 7.6 | 85.9 ± 1.6 |
| 0.061 | 84.5 ± 4.9 | 78.4 ± 9.2 | 78.1 ± 8.6 | 86.3 ± 4.1 | 85.3 ± 6.7 | 88.2 ± 1.3 |
| 0 | 93.3 ± 0.2 | 93.3 ± 0.2 | 93.3 ± 0.2 | 93.3 ± 0.2 | 93.3 ± 0.2 | 93.3 ± 0.2 |

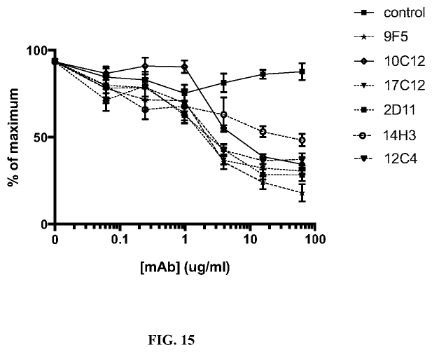

As shown in Table 56 and FIG. 15, mouse 10C12, mouse 12C4, mouse 2D11, mouse 17C12, and mouse 14H3 block neuronal internalization in a similar degree as 9F5 antibody. Internalization of tau into neurons is an important initial step in tau spreading and pathogenesis, and antibodies that block this process may share common features. Mouse 10C12, mouse 12C4, mouse 2D11, mouse 17C12, and mouse 14H3 were selected based on results in the pHrodo assay.

TABLE 56
Internalization assay results with mouse antibodies 9F5, 10C12, 17C12, 2D11, 14H3, and 12C4
All values are % of maximum signal compared to cells with no treatment

| Antibody | | | | | | | |
|---|---|---|---|---|---|---|---|
| concentration | | | | | | | |
| (μg/mL) | Control | 9F5 | 10C12 | 17C12 | 2D11 | 14H3 | 12C4 |
| 62.5 | 87.7 ± 4.8 | 18.1 ± 5 | 34.5 ± 3.7 | 30.6 ± 1.8 | 28.4 ± 3.6 | 48.4 ± 3.5 | 37.3 ± 3.5 |
| 15.625 | 86.2 ± 2.6 | 24.2 ± 3.9 | 38.9 ± 1.5 | 32.4 ± 4.8 | 28.6 ± 2.9 | 53.2 ± 3.2 | 36.6 ± 1.9 |
| 3.9 | 81.2 ± 5.3 | 35.9 ± 4.2 | 55.2 ± 1.7 | 36.8 ± 2.5 | 43 ± 3.3 | 63.1 ± 9.9 | 42.6 ± 3.2 |
| 0.98 | 75.4 ± 4.8 | 71 ± 6.0 | 90.5 ± 3.6 | 63.9 ± 4.2 | 62.7 ± 4.8 | 67.8 ± 9.7 | 69.7 ± 7.0 |
| 0.244 | 82.9 ± 1.5 | 71.7 ± 6.5 | 9.09 ± 4.9 | 78.6 ± 0.8 | 79.5 ± 8.2 | 66 ± 5.6 | 78.6 ± 5.1 |
| 0.061 | 84.5 ± 4.9 | 78.1 ± 8.6 | 86.7 ± 4.0 | 79.8 ± 4.7 | 71.5 ± 6.4 | 79.7 ± 6.8 | 78.2 ± 7.1 |
| 0 | 93.3 ± 0.2 | 93.3 ± 0.2 | 93.3 ± 0.19 | 93.7 ± 0.7 | 93.3 ± 0.2 | 93.3 ± 0.2 | 93.7 ± 0.7 |

### Example 2. Epitope Mapping of Mouse Antibodies 9F5, 2D11, 10C12, 17C12, 12C4, and 14H3 by ELISA Analysis

For ELISA mapping, peptides spanning the entire length of the tau protein were used.

Peptides contained 15 amino acids, with a 5 amino acid overlap. To enable binding to a streptavidin surface, peptides also contained an N-terminal biotin. Peptides were incubated on a streptavidin-coated plate, and the plate was blocked with 1% BSA in 1× PBS. After washing, antibodies were incubated on the plates for 1 hour at room temperature and washed. Plates were then coated with goat anti-mouse horseradish peroxidase (HRP), and washed. Plates were developed with OPD, and absorbance was read at 490 nm.

ELISA data indicate that mouse 9F5, mouse 10C12, mouse 2D11, mouse 17C12, mouse 12C4, and mouse 14H3 each strongly bind peptides containing residues 302-316 as well as residues 383-397 of the longest CNS isoform of tau (441aa, Uniprot ID P10636-8; SEQ ID NO:1). Both peptides 302-316 and 383-397 contain an “IVYK” (SEQ ID NO:276) repeat, which has been shown to be important for tau aggregation and self-association, and forms the B-sheet core of tau aggregates; in addition, it has been demonstrated as an important site for tau seeding. This may allow mouse 9F5, mouse 10C12, mouse 2D11, mouse 17C12, mouse 12C4, and mouse 14H3 to bind a range of aggregated conformers compared to other antibodies that bind regions in the MTBR. In addition, the “IVYK” (SEQ ID NO:276) repeat has been demonstrated to a important site for tau seeding, suggesting that binding with an antibody to this region may interrupt the progression of tau pathology, or be used in a diagnostic capacity to detect seed-competent species.

### Example 3. Epitope Mapping of Mouse Antibodies 9F5, 2D11, 10C12, and 17C12 by Peptide Microarray Analysis

Method

Epitope analysis of 9F5, 2D11, 10C12, and 17C12 was performed by peptide microarray analysis. The sequence of full-length human tau (441 amino acids) was linked and elongated with neutral GSGSGSG (SEQ ID NO:59) linkers at the C- and N-terminus to avoid truncated peptides. The linked and elongated antigen sequence was translated into 15 amino acid peptides with a peptide-peptide overlap of 14 amino acids. The resulting peptide microarrays contained 441 different peptides printed in duplicate (882 peptide spots), and were framed by additional HA (YPYDVPDYAG, SEQ ID NO:60, 82 spots) control peptides.

After synthesis, the microarray was blocked to prevent nonspecific binding (Rockland catalog #MB-070). Murine 9F5 was then applied to the microarray at a concentration of 1 μg/mL along with positive control mouse monoclonal anti-HA (12CA5) DyLight800 (0.5 μg/ml) for 16 h at 4° C. with shaking at 140 rpm. The microarray was washed, and secondary antibody (Goat anti-mouse IgG (H+L) DyLight680 (0.2 μg/ml) was applied for 45 minutes at room temperature. After further washing, the microarray was imaged using a Licor Odyssey Imaging System.

Quantification of spot intensities and peptide annotation were based on the 16-bit gray scale tiff files at scanning intensities of 7/7 that exhibit a higher dynamic range than the 24-bit colorized tiff files; microarray image analysis was done with PepSlide® Analyzer. A software algorithm separates fluorescence intensities of each spot into raw, foreground and background signal, and calculates averaged median foreground intensities and spot-to-spot deviations of spot duplicates (see “Raw Data” tabs). Based on averaged median foreground intensities, an intensity map was generated and interactions in the peptide map highlighted by an intensity color code with red for high and white for low spot intensities. A maximum spot-to-spot deviation of 40% was tolerated, otherwise the corresponding intensity value was zeroed.

Results

For mouse 9F5, mouse 10C12, and mouse 17C12,a very strong monoclonal antibody response was observed against two epitope-like spot patterns formed by adjacent peptides with the very similar consensus motifs QIVYKP (SEQ ID NO:57) and EIVYKSP (SEQ ID NO:58), corresponding to amino acid sequences 307-312 and 391-397 respectively of the longest CNS isoform of tau (441aa, Uniprot ID P10636-8; SEQ ID NO:1). This indicates an epitope of (Q/E)IVYK(S/P) (SEQ ID NO:56) for mouse 9F5, mouse 10C12, and mouse 17C12.

For mouse 2D11, a very strong monoclonal antibody response was observed against two epitope-like spot patterns formed by adjacent peptides with the very similar consensus motifs QIVYKP (SEQ ID NO:57) and EIVYKS (SEQ ID NO:277), corresponding to amino acid sequences 307-312 and 391-396 respectively of the longest CNS isoform of tau (441aa, Uniprot ID P10636-8; SEQ ID NO:1). This indicates an epitope of (Q/E)IVYK(S/P) (SEQ ID NO:56) for mouse 2D11.

### Example 4. Design of Humanized 9F5 Antibodies

The starting point or donor antibody for humanization was the mouse antibody 9F5. The heavy chain variable amino acid sequence of mature m9F5 is provided as SEQ ID NO:7. The light chain variable amino acid sequence of mature m9F5 is provided as SEQ ID NO:11. The heavy chain Kabat/Chothia Composite CDR1, CDR2, and CDR3 amino acid sequences are provided as SEQ ID NOs:8-10, respectively. The light chain Kabat CDR1, CDR2, and CDR3 amino acid sequences are provided as SEQ ID NOs12-14 respectively. Kabat numbering is used throughout.

The variable kappa (Vk) of 9F5 belongs to mouse Kabat subgroup 2 which corresponds to human Kabat subgroup 2 and the variable heavy (Vh) to mouse Kabat subgroup 2c which corresponds to human Kabat subgroup 1 [Kabat E. A., et al., (1991), Sequences of Proteins of Immunological Interest, Fifth Edition. NIH Publication No. 91-3242]. 16 residue Chothia CDR-L1 is similar to Chothia canonical class 4, 7 residue Chothia CDR-L2 is of Chothia canonical class 1, 9 residue Chothia CDR-L3 is similar to Chothia canonical class 1 [Martin ACR. (2010) Protein sequence and structure analysis of antibody variable domains. In: Kontermann R and Dübel S (eds). Antibody Engineering. Heidelberg, Germany: Springer International Publishing AG. [Martin, 2010]. 10 residue Chothia CDR-H1 is similar to Chothia canonical class 1, 17 residue Chothia CDR-H2 is similar to Chothia canonical class 2 [Martin, 2010]]. 3 residue CDR-H3 has no canonical classes. A search was made over the protein sequences in the PDB database [Deshpande N, et al., (2005) Nucleic Acids Res. 33: D233-7] to find structures, which would provide a rough structural model of 9F5. The crystal structure of an antibody fab pdb code 5OBF [Vicentini, et al., 2017, unpublished] was used for both Vh and Vk structure since it had good resolution (1.92A°) and overall sequence similarity to 9F5 Vh and Vk, retaining the same canonical structures for the loops.

The frameworks of 9F5 VH share a high degree of sequence similarity with the corresponding regions of humanized 48G7 Fab PDB: 2RCS, designed by Wedemayer, G. J. et al. (1997, Science 276: 1665-1669) and heavy chain AAN16432 cloned by McElhiney, J. et al (2002, Appl. Environ. Microbiol. 68 (11), 5288-5295). The variable domains of 9F5 and AAN16432 & 48G7 fab also share identical lengths for the CDR-H1, H2 loops. Similarly, the frameworks of 9F5 VL share a high degree of sequence similarity with the corresponding regions of human antibody CAB51297 VL and 1911357B VL, cloned by Capello et al (GenBank Ref. NO. CAB51297, submitted Jul. 20, 1999, unpublished) and Cooper L.J, et al. (1993, J. Immunol. 150 (6), 2231-2242), respectively. The variable light domain of 9F5 and CAB51297 & 1911357B antibodies also share identical lengths for the CDR-L1, L2 and L3 loops. Accordingly, the framework regions of AAN16432 VH & 48G7 VH (2RCS-VH) and CAB51297 VL & 1911357B VL were chosen as the hybrid acceptor sequences for the CDRs of 9F5. A model of the 9F5 CDRs grafted onto the respective human frame-works for VH and VL was built and used as a guidance for further backmutations.

Heavy and light chain variant sequences resulting from antibody humanization process were further aligned to human germ line sequences using IMGT Domain GapAlign tool to assess the humanness of the heavy and light chain as outlined by WHO INN committee guidelines. (WHO-INN: International nonproprietary names (INN) for biological and biotechnological substances (a review) (Internet) 2014. Available from: http://www.who.int/medicines/services/inn/BioRev2014.pdf) Residues were changed to align with corresponding human germ line sequence, where possible, to enhance humanness and to reduce potential immunogenicity. For humanized VLv2, VLv3, VLv4, VLv5, VLv6, VLv7, VLv8, and VLv9 variants, mutations were introduced to render the sequences more similar to human germ line gene IGKV2-28*01 & IGKJ2*01 (SEQ ID NO:37). For humanized VHv2, VHv3, VHv4, VHv5, VHv6, VHv7, VHv8, VHv9, and VHv10 variants, mutations were introduced to render the sequences more similar to human germ line gene IGHV1-69-2*01 (SEQ ID NO:33)

Additional versions of hu9F5-VH and hu9F5-VL were designed to enable assessment of various framework residues for their contributions to antigen binding, thermostability, and immunogenicity, and for optimization of deamination, oxidation, N-glycosylation, proteolysis and aggregation. The positions considered for mutation include those that . . . . The positions considered for mutation include those that . . . .
define the canonical CDR conformations (summarized in Martin, A. C. R. (2010) Protein sequence and structure analysis of antibody variable domains. In: Kontermann R and Dübel S (eds). Antibody Engineering. Heidelberg, Germany: Springer International Publishing AG.),
are within the Vernier zone (Foote J and Winter G. (1992) Antibody framework residues affecting the conformation of the hypervariable loops. J Mol Biol. 224(2):487-99.),
localize to the VH/VL domain interface (summarized in Léger O J P and Saldanha J.

(2000) Preparation of recombinant antibodies from immune rodent spleens and the design of their humanisation by CDR grafting. In: Shepherd P and Dean C (eds). Monoclonal Antibodies: a Practical Approach. Oxford, UK: Oxford University Press.),
are susceptible to post-translational modifications, such as glycosylation or pyroglutamination, are occupied by residues that are predicted to clash with CDRs, according to the model of 9F5 CDRs grafted onto VH and VL frameworks, or
are occupied by residues that are rare among sequenced human antibodies, where either the parental mouse 9F5 residue or some other residue is much more prevalent within human antibody repertoire.

Alignments of the murine 9F5 and various humanized antibodies are shown for the light chain variable regions (Table 7 and FIGS. 2A-2B, 5A-5B, 6A-6C), and heavy chain variable regions (Table 6 and FIGS. 1A-1B, 4A-4B).

10 humanized heavy chain variable region variants and 9 humanized light chain variable region variants were constructed containing different permutations of substitutions: hu9F5VHv1, hu9F5VHv2, hu9F5VHv3, hu9F5VHv4, hu9F5VHv5, hu9F5VHv6, hu9F5VHv7, hu9F5VHv8, hu9F5VHv9, or 9F5VHv10 (SEQ ID NOs:15-22, SEQ ID NOs:127-128, respectively); and hu9F5VLv1, hu9F5VLv2, hu9F5VLv3, hu9F5VLv4, hu9F5VLv5, hu9F5VLv6, hu9F5VLv7, hu9F5VLv8, or hu9F5VLv9 (SEQ ID NOs:23-29, SEQ ID NOs:130-131, respectively) (Tables 6 and 7). The exemplary humanized Vk and Vh designs, with backmutations and other mutations based on selected human frameworks, are shown in Tables 6 and 7, respectively. The bolded areas in Tables 6 and 7 indicate the CDRs as defined by Kabat/Chothia Composite. A “-” in the columns in Tables 6 and 7 indicates no residue at the indicated position. SEQ ID NOs:15-22 and SEQ ID NOs:127-128, and SEQ ID NOs:23-29 and SEQ ID NOs:130-131 contain backmutations and other mutations as shown in Table 8. The amino acids at positions in hu9F5VHv1, hu9F5VHv2, hu9F5VHv3, hu9F5VHv4, hu9F5VHv5, hu9F5VHv6, hu9F5VHv7, hu9F5VHv8, hu9F5VHv9, and hu9F5VHv10 are listed in Table 9. The amino acids at positions in hu9F5VLv1, hu9F5VLv2, hu9F5VLv3, hu9F5VLv4, hu9F5VLv5, hu9F5VLv6, hu9F5VLv7, hu9F5VLv8, and hu9F5VLv9 are listed in Table 10. Hu9F5VLv8 with substitution N60D is also known as hu9F5VLv9.

The percentage humanness for humanized VH chains hu9F5VHv1, hu9F5VHv2, hu9F5VHv3, hu9F5VHv4, hu9F5VHv5, hu9F5VHv6, hu9F5VHv7, hu9F5VLv8, hu9F5VHv9, and hu9F5VHv10 (SEQ ID NOs:15-22 and SEQ ID NOs: 127-128, respectively) with respect to the most similar human germline gene IGHV1-69-2*01 (SEQ ID NO:33), and for humanized VL chains hu9F5VLv1, hu9F5VLv2, hu9F5VLv3, hu9F5VLv4, hu9F5VLv5, hu9F5VLv6, hu9F5VLv7, hu9F5VLv8, and hu9F5VLv9 (SEQ ID NOs:23-29 and SEQ ID NOs:130-131, respectively) with respect to the most similar human germline gene IGKV2-28*01 & IGKJ2*01 (SEQ ID NO:37), is shown in Table 11.

TABLE 6

| | | | Mouse | IMGT# | | | | | | | | | | | | |
|---|---|---|---|---|---|---|---|---|---|---|---|---|---|---|---|---|
| | | | 9F5VH | IGHV1- | AAN16432— | | | | | | | | | | | |
| Kabat | Linear | | (SEQ | 69-2*01 | VH_huFrwk | 2RCS-VH_ | Hu9F5 | Hu9F5 | Hu9F5 | | | | | | | Hu9F |
| resi- | resi- | FR | ID | (SEQ | (SEQ | huFrwk | VHv1 | VHv2 | VHv3 | Hu9F5VHvv4 | Hu9F5VHwvv | Hu9F5VHv6 | Hu9F5VHv7 | Hu9F5VHv8 | Hu9F5VHv9 | 5VHv10 |
| due | due | or | NO: | ID | ID | (SEQ ID | (SEQ ID | (SEQ ID | (SEQ ID | (SEQ ID | (SEQ ID | (SEQ ID | (SEQ ID | (SEQ ID | (SEQ ID | (SEQ ID |
| # | # | CDR | 7) | NO: 33) | NO: 31) | NO: 32) | NO: 15) | NO: 16) | NO: 17) | NO: 18) | NO: 19) | NO: 20) | NO: 21) | NO: 22) | NO: 127) | NO: 128) |
| 1 | 1 | Fr1 | E | E | E | Q | Q | E | E | E | E | E | E | E | E | E |
| 2 | 2 | Fr1 | V | V | V | V | V | V | V | V | V | V | V | V | V | V |
| 3 | 3 | Fr1 | Q | Q | Q | Q | Q | Q | Q | Q | Q | Q | Q | Q | Q | Q |
| 4 | 4 | Fr1 | L | L | L | L | L | L | L | L | L | L | L | L | L | L |
| 5 | 5 | Fr1 | Q | V | V | Q | Q | Q | Q | Q | Q | Q | V | V | V | V |
| 6 | 6 | Fr1 | Q | Q | Q | Q | Q | Q | Q | Q | Q | Q | Q | Q | Q | Q |
| 7 | 7 | Fr1 | S | S | S | S | S | S | S | S | S | S | S | S | S | S |
| 8 | 8 | Fr1 | G | G | G | G | G | G | G | G | G | G | G | G | G | G |
| 9 | 9 | Fr1 | A | A | A | A | A | A | A | A | A | A | A | A | A | A |
| 10 | 10 | Fr1 | E | E | E | E | E | E | E | E | E | E | E | E | E | E |
| 11 | 11 | Fr1 | L | V | V | L | L | L | L | L | L | L | V | V | V | V |
| 12 | 12 | Fr1 | V | K | K | V | V | V | V | V | V | V | K | K | K | K |
| 13 | 13 | Fr1 | R | K | K | K | K | K | K | K | K | K | K | K | K | K |
| 14 | 14 | Fr1 | P | P | P | P | P | P | P | P | P | P | P | P | P | P |
| 15 | 15 | Fr1 | G | G | G | G | G | G | G | G | G | G | G | G | G | G |
| 16 | 16 | Fr1 | A | A | A | A | A | A | A | A | A | A | A | A | A | A |
| 17 | 17 | Fr1 | S | T | S | S | S | T | T | T | T | T | T | T | T | T |
| 18 | 18 | Fr1 | V | V | V | V | V | V | V | V | V | V | V | V | V | V |
| 19 | 19 | Fr1 | K | K | K | K | K | K | K | K | K | K | K | K | K | K |
| 20 | 20 | Fr1 | L | I | V | L | L | I | I | I | I | I | I | I | I | I |
| 21 | 21 | Fr1 | S | S | S | S | S | S | S | S | S | S | S | S | S | S |
| 22 | 22 | Fr1 | C | C | C | C | C | C | C | C | C | C | C | C | C | C |
| 23 | 23 | Fr1 | T | K | K | T | T | T | T | K | K | K | K | K | K | K |
| 24 | 24 | Fr1 | A | V | V | A | A | A | A | A | A | A | A | A | A | A |
| 25 | 25 | Fr1 | S | S | S | S | S | S | S | S | S | S | S | S | S | S |
| 26 | 26 | CDR- | G | G | G | G | G | G | G | G | G | G | G | G | G | G |
| | | H1 | | | | | | | | | | | | | | |
| 27 | 27 | CDR- | F | Y | Y | F | F | F | F | F | F | F | F | F | F | F |
| | | H1 | | | | | | | | | | | | | | |
| 28 | 28 | CDR- | N | T | T | N | N | N | N | T | T | T | N | N | N | N |
| | | H1 | | | | | | | | | | | | | | |
| 29 | 29 | CDR- | I | F | L | I | I | I | I | I | I | I | I | I | I | I |
| | | H1 | | | | | | | | | | | | | | |
| 30 | 30 | CDR- | K | T | T | K | K | K | K | K | K | K | K | K | K | K |
| | | H1 | | | | | | | | | | | | | | |
| 31 | 31 | CDR- | D | D | E | D | D | D | D | D | D | D | D | D | D | D |
| | | H1 | | | | | | | | | | | | | | |
| 32 | 32 | CDR- | D | Y | L | T | D | D | D | D | D | D | D | D | D | D |
| | | H1 | | | | | | | | | | | | | | |
| 33 | 33 | CDR- | Y | Y | S | Y | Y | Y | Y | Y | Y | Y | Y | Y | Y | Y |
| | | H1 | | | | | | | | | | | | | | |
| 34 | 34 | CDR- | M | M | M | M | M | M | M | M | M | M | M | M | M | M |
| | | H1 | | | | | | | | | | | | | | |
| 35 | 35 | CDR- | N | H | H | H | N | N | N | N | N | N | N | N | N | N |
| | | H1 | | | | | | | | | | | | | | |
| 35A | | CDR- | — | | — | — | — | — | — | — | — | — | — | | | |
| | | H1 | | | | | | | | | | | | | | |
| 35B | | CDR- | — | | — | — | — | — | — | — | — | — | — | | | |
| | | H1 | | | | | | | | | | | | | | |
| 36 | 36 | Fr2 | W | W | W | W | W | W | W | W | W | W | W | W | W | W |
| 37 | 37 | Fr2 | V | V | V | V | V | V | V | V | V | V | V | V | V | V |
| 38 | 38 | Fr2 | K | Q | R | K | K | K | K | K | K | K | R | R | Q | K |
| 39 | 39 | Fr2 | Q | Q | Q | Q | Q | Q | Q | Q | Q | Q | Q | Q | Q | Q |
| 40 | 40 | Fr2 | R | A | A | R | R | R | R | R | R | A | A | A | R | R |
| 41 | 41 | Fr2 | P | P | P | P | P | P | P | P | P | P | P | P | P | P |
| 42 | 42 | Fr2 | E | G | G | E | E | E | E | E | E | E | G | G | G | E |
| 43 | 43 | Fr2 | R | K | K | Q | Q | Q | Q | Q | K | K | K | K | K | K |
| 44 | 44 | Fr2 | G | G | G | G | G | G | G | G | G | G | G | G | G | G |
| 45 | 45 | Fr2 | L | L | L | L | L | L | L | L | L | L | L | L | L | L |
| 46 | 46 | Fr2 | E | E | E | E | E | E | E | E | E | E | E | E | E | E |
| 47 | 47 | Fr2 | W | W | W | W | W | W | W | W | W | W | W | W | W | W |
| 48 | 48 | Fr2 | I | M | M | I | I | I | I | I | I | M | I | I | I | I |
| 49 | 49 | Fr2 | G | G | G | G | G | G | G | G | G | G | G | G | G | G |
| 50 | 50 | CDR- | W | L | G | R | W | W | W | W | W | W | W | W | W | W |
| | | H2 | | | | | | | | | | | | | | |
| 51 | 51 | CDR- | I | V | F | I | I | I | I | I | V | V | V | V | I | I |
| | | H2 | | | | | | | | | | | | | | |
| 52 | 52 | CDR- | D | D | D | D | D | D | D | D | D | D | D | D | D | D |
| | | H2 | | | | | | | | | | | | | | |
| 52A | 53 | CDR- | P | P | P | P | P | P | P | P | P | P | P | P | P | P |
| | | H2 | | | | | | | | | | | | | | |
| 52B | | CDR- | — | — | — | — | — | — | — | — | — | — | — | — | | |
| | | H2 | | | | | | | | | | | | | | |
| 52C | | CDR- | — | — | — | — | — | — | — | — | — | — | — | — | | |
| | | H2 | | | | | | | | | | | | | | |
| 53 | 54 | CDR- | E | E | E | A | E | E | E | E | E | E | E | E | E | E |
| | | H2 | | | | | | | | | | | | | | |
| 54 | 55 | CDR- | N | D | D | N | N | N | N | N | D | D | D | N | N | N |
| | | H2 | | | | | | | | | | | | | | |
| 55 | 56 | CDR- | G | G | G | G | G | G | G | G | G | G | G | G | G | G |
| | | H2 | | | | | | | | | | | | | | |
| 56 | 57 | CDR- | D | E | E | N | D | D | D | D | E | E | E | D | D | D |
| | | H2 | | | | | | | | | | | | | | |
| 57 | 58 | CDR- | T | T | T | T | T | T | T | T | T | T | T | T | T | T |
| | | H2 | | | | | | | | | | | | | | |
| 58 | 59 | CDR- | E | I | I | K | E | E | E | E | E | E | E | E | E | E |
| | | H2 | | | | | | | | | | | | | | |
| 59 | 60 | CDR- | Y | Y | Y | Y | Y | Y | Y | Y | Y | Y | Y | Y | Y | Y |
| | | H2 | | | | | | | | | | | | | | |
| 60 | 61 | CDR- | A | A | A | D | A | A | A | A | A | A | A | A | A | A |
| | | H2 | | | | | | | | | | | | | | |
| 61 | 62 | CDR- | S | E | Q | P | S | S | S | S | S | S | S | S | S | S |
| | | H2 | | | | | | | | | | | | | | |
| 62 | 63 | CDR- | K | K | K | K | K | K | K | K | K | K | K | K | K | K |
| | | H2 | | | | | | | | | | | | | | |
| 63 | 64 | CDR- | F | F | F | F | F | F | F | F | F | F | F | F | F | F |
| | | H2 | | | | | | | | | | | | | | |
| 64 | 65 | CDR- | Q | Q | Q | Q | Q | Q | Q | Q | Q | Q | Q | Q | Q | Q |
| | | H2 | | | | | | | | | | | | | | |
| 65 | 66 | CDR- | G | G | G | G | G | G | G | G | G | G | G | G | G | G |
| | | H2 | | | | | | | | | | | | | | |
| 66 | 67 | Fr3 | K | R | R | K | K | K | R | R | R | R | R | R | R | R |
| 67 | 68 | Fr3 | A | V | V | A | A | A | A | A | A | A | A | A | A | A |
| 68 | 69 | Fr3 | T | T | T | T | T | T | T | T | T | T | T | T | T | T |
| 69 | 70 | Fr3 | M | I | M | I | I | M | M | M | M | M | M | M | M | M |
| 70 | 71 | Fr3 | T | T | T | T | T | T | T | T | T | T | T | T | T | T |
| 71 | 72 | Fr3 | A | A | E | A | A | A | A | A | A | A | A | A | A | A |
| 72 | 73 | Fr3 | D | D | D | D | D | D | D | D | D | D | D | D | D | D |
| 73 | 74 | Fr3 | T | T | T | T | T | T | T | T | T | T | T | T | T | T |
| 74 | 75 | Fr3 | S | S | S | S | S | S | S | S | S | S | S | S | S | S |
| 75 | 76 | Fr3 | S | T | T | S | S | T | T | T | T | T | T | T | T | T |
| 76 | 77 | Fr3 | N | D | D | N | N | N | N | N | D | D | D | D | N | N |
| 77 | 78 | Fr3 | T | T | T | T | T | T | T | T | T | T | T | T | T | T |
| 78 | 79 | Fr3 | A | A | A | A | A | A | A | A | A | A | A | A | A | A |
| 79 | 80 | Fr3 | Y | Y | Y | Y | Y | Y | Y | Y | Y | Y | Y | Y | Y | Y |
| 80 | 81 | Fr3 | L | M | M | L | L | L | L | L | M | M | M | M | M | M |
| 81 | 82 | Fr3 | Q | E | E | Q | Q | Q | E | E | E | E | E | E | E | E |
| 82 | 83 | Fr3 | F | L | L | L | L | L | L | L | L | L | L | L | L | L |
| 82A | 84 | Fr3 | S | S | S | S | S | S | S | S | S | S | S | S | S | S |
| 82B | 85 | Fr3 | S | S | S | S | S | S | S | S | S | S | S | S | S | S |
| 82C | 86 | Fr3 | L | L | L | L | L | L | L | L | L | L | L | L | L | L |
| 83 | 87 | Fr3 | T | R | R | T | T | T | T | R | R | R | R | R | R | R |
| 84 | 88 | Fr3 | S | S | S | S | S | S | S | S | S | S | S | S | S | S |
| 85 | 89 | Fr3 | E | E | E | E | E | E | E | E | E | E | E | E | E | E |
| 86 | 90 | Fr3 | D | D | D | D | D | D | D | D | D | D | D | D | D | D |
| 87 | 91 | Fr3 | T | T | T | T | T | T | T | T | T | T | T | T | T | T |
| 88 | 92 | Fr3 | A | A | A | A | A | A | A | A | A | A | A | A | A | A |
| 89 | 93 | Fr3 | V | V | V | V | V | V | V | V | V | V | V | V | V | V |
| 90 | 94 | Fr3 | Y | Y | Y | Y | Y | Y | Y | Y | Y | Y | Y | Y | Y | Y |
| 91 | 95 | Fr3 | Y | Y | Y | Y | Y | Y | Y | Y | Y | Y | Y | Y | Y | Y |
| 92 | 96 | Fr3 | C | C | C | C | C | C | C | C | C | C | C | C | C | C |
| 93 | 97 | Fr3 | T | A | A | A | A | T | T | T | T | T | T | T | T | T |
| 94 | 98 | Fr3 | T | T | G | S | S | T | T | T | T | T | T | T | T | T |
| 95 | 99 | CDR- | S | — | Y | Y | S | S | S | S | S | S | S | S | S | S |
| | | H3 | | | | | | | | | | | | | | |
| 96 | | CDR- | — | — | R | Y | — | — | — | — | — | — | — | — | | |
| | | H3 | | | | | | | | | | | | | | |
| 97 | | CDR- | — | — | S | G | — | — | — | — | — | — | — | — | | |
| | | H3 | | | | | | | | | | | | | | |
| 98 | | CDR- | — | — | P | — | — | — | — | — | — | — | — | — | | |
| | | H3 | | | | | | | | | | | | | | |
| 99 | | CDR- | — | — | M | — | — | — | — | — | — | — | — | — | | |
| | | H3 | | | | | | | | | | | | | | |
| 100 | | CDR- | — | — | P | — | — | — | — | — | — | — | — | — | | |
| | | H3 | | | | | | | | | | | | | | |
| 100A | | CDR- | — | — | T | — | — | — | — | — | — | — | — | — | | |
| | | H3 | | | | | | | | | | | | | | |
| 100B | | CDR- | — | — | A | — | — | — | — | — | — | — | — | — | | |
| | | H3 | | | | | | | | | | | | | | |
| 100C | | CDR- | — | — | — | — | — | — | — | — | — | — | — | — | | |
| | | H3 | | | | | | | | | | | | | | |
| 100D | | CDR- | — | — | — | — | — | — | — | — | — | — | — | — | | |
| | | H3 | | | | | | | | | | | | | | |
| 100E | | CDR- | — | — | — | — | — | — | — | — | — | — | — | — | | |
| | | H3 | | | | | | | | | | | | | | |
| 100F | | CDR- | — | — | — | — | — | — | — | — | — | — | — | — | | |
| | | H3 | | | | | | | | | | | | | | |
| 100G | | CDR- | — | — | — | — | — | — | — | — | — | — | — | — | | |
| | | H3 | | | | | | | | | | | | | | |
| 100H | | CDR- | — | — | — | — | — | — | — | — | — | — | — | — | | |
| | | H3 | | | | | | | | | | | | | | |
| 100I | | CDR- | — | — | — | — | — | — | — | — | — | — | — | — | | |
| | | H3 | | | | | | | | | | | | | | |
| 100J | | CDR- | — | — | — | — | — | — | — | — | — | — | — | — | | |
| | | H3 | | | | | | | | | | | | | | |
| 100K | | CDR- | — | — | — | — | — | — | — | — | — | — | — | — | | |
| | | H3 | | | | | | | | | | | | | | |
| 101 | 100 | CDR- | N | Q | N | I | N | N | N | N | N | N | N | N | N | N |
| | | H3 | | | | | | | | | | | | | | |
| 102 | 101 | CDR- | G | H | K | Y | G | G | G | G | G | G | G | G | G | G |
| | | H3 | | | | | | | | | | | | | | |
| 103 | 102 | Fr4 | W | W | W | W | W | W | W | W | W | W | W | W | W | W |
| 104 | 103 | Fr4 | G | G | G | G | G | G | G | G | G | G | G | G | G | G |
| 105 | 104 | Fr4 | Q | Q | Q | Q | Q | Q | Q | Q | Q | Q | Q | Q | Q | Q |
| 106 | 105 | Fr4 | G | G | G | G | G | G | G | G | G | G | G | G | G | G |
| 107 | 106 | Fr4 | T | T | T | T | T | T | T | T | T | T | T | T | T | T |
| 108 | 107 | Fr4 | L | L | L | T | T | T | T | T | L | L | L | L | L | L |
| 109 | 108 | Fr4 | V | V | V | L | L | V | V | V | V | V | V | V | V | V |
| 110 | 109 | Fr4 | T | T | T | T | T | T | T | T | T | T | T | T | T | T |
| 111 | 110 | Fr4 | V | V | V | V | V | V | V | V | V | V | V | V | V | V |
| 112 | 111 | Fr4 | S | S | S | S | S | S | S | S | S | S | S | S | S | S |
| 113 | 112 | Fr4 | TS | S | S | S | S | S | S | S | S | S | S | S | S | |

TABLE 7

| | | | | IGKV2-28* | | | | | | | | | | | |
|---|---|---|---|---|---|---|---|---|---|---|---|---|---|---|---|
| Kabat | | | Mouse | 01 & | CAB51297- | 1911357B- | | | | | | | | | Hu9F5 |
| resi- | Linear | FR | 9F5 VL | IGKJ2*01 | VL_huFrwk | VL_huFrwk | Hu9F5VLv1 | Hu9F5VLv2 | Hu9F5VLv3 | Hu9F5VLvv4 | Hu9F5VLv5 | Hu9F5VLv6 | Hu9F5VLv7 | Hu9F5VLv8 | VLv9 |
| due | residue | or | (SEQ ID | (SEQ ID | (SEQ ID | (SEQ ID | (SEQ ID | (SEQ ID | (SEQ ID | (SEQ ID | (SEQ ID | (SEQ ID | (SEQ ID | (SEQ ID | (SEQ ID |
| # | # | CDR | NO: 11) | NO: 37) | NO: 35) | NO: 36) | NO: 23) | NO: 24) | NO: 25) | NO: 26) | NO: 27) | NO: 28) | NO: 29) | NO: 130) | NO: 131) |
| 1 | 1 | Fr1 | D | D | D | D | D | D | D | D | D | D | D | D | D |
| 2 | 2 | Fr1 | I | I | I | I | I | I | I | I | I | I | I | I | I |
| 3 | 3 | Fr1 | V | V | V | V | V | V | V | V | V | V | V | V | V |
| 4 | 4 | Fr1 | M | M | M | M | M | M | M | M | M | M | M | M | M |
| 5 | 5 | Fr1 | T | T | T | T | T | T | T | T | T | T | T | T | T |
| 6 | 6 | Fr1 | Q | Q | Q | Q | Q | Q | Q | Q | Q | Q | Q | Q | Q |
| 7 | 7 | Fr1 | A | S | S | A | A | S | S | S | S | S | S | S | S |
| 8 | 8 | Fr1 | A | P | P | A | A | P | P | P | P | P | P | P | P |
| 9 | 9 | Fr1 | F | L | L | F | F | F | F | F | F | F | L | F | F |
| 10 | 10 | Fr1 | S | S | S | S | S | S | S | S | S | S | S | S | S |
| 11 | 11 | Fr1 | N | L | L | N | N | N | N | L | L | L | L | L | L |
| 12 | 12 | Fr1 | P | P | P | P | P | P | P | P | P | P | P | P | P |
| 13 | 13 | Fr1 | V | V | V | V | V | V | V | V | V | V | V | V | V |
| 14 | 14 | Fr1 | T | T | T | T | T | T | T | T | T | T | T | T | T |
| 15 | 15 | Fr1 | L | P | P | L | L | P | P | P | P | P | P | P | P |
| 16 | 16 | Fr1 | G | G | G | G | G | G | G | G | G | G | G | G | G |
| 17 | 17 | Fr1 | T | E | E | T | T | T | E | E | E | E | E | E | E |
| 18 | 18 | Fr1 | S | P | S | S | S | S | S | S | S | S | S | S | S |
| 19 | 19 | Fr1 | A | A | A | A | A | A | A | A | A | A | A | A | A |
| 20 | 20 | Fr1 | S | S | S | S | S | S | S | S | S | S | S | S | S |
| 21 | 21 | Fr1 | I | I | I | I | I | I | I | I | I | I | I | I | I |
| 22 | 22 | Fr1 | S | S | S | S | S | S | S | S | S | S | S | S | S |
| 23 | 23 | Fr1 | C | C | C | C | C | C | C | C | C | C | C | C | C |
| 24 | 24 | CDR- | R | R | R | R | R | R | R | R | R | R | R | R | R |
| | | L1 | | | | | | | | | | | | | |
| 25 | 25 | CDR- | S | S | S | S | S | S | S | S | S | S | S | S | S |
| | | L1 | | | | | | | | | | | | | |
| 26 | 26 | CDR- | S | S | S | S | S | S | S | S | S | S | S | S | S |
| | | L1 | | | | | | | | | | | | | |
| 27 | 27 | CDR- | K | Q | Q | K | K | K | K | K | K | K | K | K | K |
| | | L1 | | | | | | | | | | | | | |
| 27A | 28 | CDR- | S | S | S | N | S | S | S | S | S | S | S | S | S |
| | | L1 | | | | | | | | | | | | | |
| 27B | 29 | CDR- | L | L | L | L | L | L | L | L | L | L | L | L | L |
| | | L1 | | | | | | | | | | | | | |
| 27C | 30 | CDR- | L | L | L | L | L | L | L | L | L | L | L | L | L |
| | | L1 | | | | | | | | | | | | | |
| 27D | 31 | CDR- | H | H | H | H | H | H | H | H | H | H | H | H | H |
| | | L1 | | | | | | | | | | | | | |
| 27E | 32 | CDR- | S | S | S | S | S | S | S | S | S | S | S | S | S |
| | | L1 | | | | | | | | | | | | | |
| 27F | | CDR- | — | — | | — | — | — | — | — | — | — | — | | |
| | | L1 | | | | | | | | | | | | | |
| 28 | 33 | CDR- | N | N | N | N | N | N | N | N | N | N | N | N | N |
| | | L1 | | | | | | | | | | | | | |
| 29 | 34 | CDR- | G | G | G | G | G | G | G | G | G | G | G | G | G |
| | | L1 | | | | | | | | | | | | | |
| 30 | 35 | CDR- | I | Y | Y | I | I | I | I | I | Y | Y | I | I | I |
| | | L1 | | | | | | | | | | | | | |
| 31 | 36 | CDR- | T | N | N | T | T | T | T | T | T | T | N | T | T |
| | | L1 | | | | | | | | | | | | | |
| 32 | 37 | CDR- | Y | Y | Y | F | Y | Y | Y | Y | Y | Y | Y | Y | Y |
| | | L1 | | | | | | | | | | | | | |
| 33 | 38 | CDR- | L | L | L | L | L | L | L | L | L | L | L | L | L |
| | | L1 | | | | | | | | | | | | | |
| 34 | 39 | CDR- | Y | D | D | Y | Y | Y | Y | Y | Y | Y | Y | Y | Y |
| | | L1 | | | | | | | | | | | | | |
| 35 | 40 | Fr2 | W | W | W | W | W | W | W | W | W | W | W | W | W |
| 36 | 41 | Fr2 | Y | Y | Y | Y | Y | Y | Y | Y | Y | Y | Y | Y | Y |
| 37 | 42 | Fr2 | L | L | L | L | L | L | L | L | L | L | L | L | L |
| 38 | 43 | Fr2 | Q | Q | Q | Q | Q | Q | Q | Q | Q | Q | Q | Q | Q |
| 39 | 44 | Fr2 | K | K | K | R | R | R | R | R | R | R | K | K | K |
| 40 | 45 | Fr2 | P | P | P | P | P | P | P | P | P | P | P | P | P |
| 41 | 46 | Fr2 | G | G | G | G | G | G | G | G | G | G | G | G | G |
| 42 | 47 | Fr2 | Q | Q | Q | Q | Q | Q | Q | Q | Q | Q | Q | Q | Q |
| 43 | 48 | Fr2 | S | S | S | S | S | S | S | S | S | S | S | S | S |
| 44 | 49 | Fr2 | P | P | P | P | P | P | P | P | P | P | P | P | P |
| 45 | 50 | Fr2 | Q | Q | Q | Q | Q | Q | Q | Q | Q | Q | Q | Q | Q |
| 46 | 51 | Fr2 | L | L | L | L | L | L | L | L | L | L | L | L | L |
| 47 | 52 | Fr2 | L | L | L | L | L | L | L | L | L | L | L | L | L |
| 48 | 53 | Fr2 | I | I | I | I | I | I | I | I | I | I | I | I | I |
| 49 | 54 | Fr2 | Y | Y | Y | Y | Y | Y | Y | Y | Y | Y | Y | Y | Y |
| 50 | 55 | CDR- | Q | L | L | R | Q | Q | Q | Q | Q | Q | Q | Q | Q |
| | | L2 | | | | | | | | | | | | | |
| 51 | 56 | CDR- | M | G | G | V | M | M | M | G | G | G | G | M | M |
| | | L2 | | | | | | | | | | | | | |
| 52 | 57 | CDR- | S | S | S | S | S | S | S | S | S | S | S | S | S |
| | | L2 | | | | | | | | | | | | | |
| 53 | 58 | CDR- | N | N | N | N | N | N | N | N | N | N | N | N | N |
| | | L2 | | | | | | | | | | | | | |
| 54 | 59 | CDR- | L | R | R | L | L | L | L | R | R | R | R | L | L |
| | | L2 | | | | | | | | | | | | | |
| 55 | 60 | CDR- | A | A | A | A | A | A | A | A | A | A | A | A | A |
| | | L2 | | | | | | | | | | | | | |
| 56 | 61 | CDR- | S | S | S | S | S | S | S | S | S | S | S | S | S |
| | | L2 | | | | | | | | | | | | | |
| 57 | 62 | Fr3 | G | G | G | G | G | G | G | G | G | G | G | G | G |
| 58 | 63 | Fr3 | V | V | V | V | V | V | V | V | V | V | V | V | V |
| 59 | 64 | Fr3 | P | P | P | P | P | P | P | P | P | P | P | P | P |
| 60 | 65 | Fr3 | D | D | D | N | N | N | N | N | N | N | D | N | D |
| 61 | 66 | Fr3 | R | R | R | R | R | R | R | R | R | R | R | R | R |
| 62 | 67 | Fr3 | F | F | F | F | F | F | F | F | F | F | F | F | F |
| 63 | 68 | Fr3 | S | S | S | S | S | S | S | S | S | S | S | S | S |
| 64 | 69 | Fr3 | S | G | G | G | S | S | G | G | G | G | G | S | S |
| 65 | 70 | Fr3 | S | S | S | S | S | S | S | S | S | S | S | S | S |
| 66 | 71 | Fr3 | G | G | G | E | G | G | G | G | G | E | G | G | G |
| 67 | 72 | Fr3 | S | S | S | S | S | S | S | S | S | S | S | S | S |
| 68 | 73 | Fr3 | G | G | G | G | G | G | G | G | G | G | G | G | G |
| 69 | 74 | Fr3 | T | T | T | T | T | T | T | T | T | T | T | T | T |
| 70 | 75 | Fr3 | D | D | D | D | D | D | D | D | D | D | D | D | D |
| 71 | 76 | Fr3 | F | F | F | F | F | F | F | F | F | F | F | F | F |
| 72 | 77 | Fr3 | T | T | T | T | T | T | T | T | T | T | T | T | T |
| 73 | 78 | Fr3 | L | L | L | L | L | L | L | L | L | L | L | L | L |
| 74 | 79 | Fr3 | R | K | K | R | R | R | R | R | R | R | K | K | K |
| 75 | 80 | Fr3 | I | I | I | I | I | I | I | I | I | I | I | I | I |
| 76 | 81 | Fr3 | S | S | S | S | S | S | S | S | S | S | S | S | S |
| 77 | 82 | Fr3 | R | R | R | R | R | R | R | R | R | R | R | R | R |
| 78 | 83 | Fr3 | V | V | V | V | V | V | V | V | V | V | V | V | V |
| 79 | 84 | Fr3 | E | E | E | E | E | E | E | E | E | E | E | E | E |
| 80 | 85 | Fr3 | A | A | A | A | A | A | A | A | A | A | A | A | A |
| 81 | 86 | Fr3 | E | E | E | E | E | E | E | E | E | E | E | E | E |
| 82 | 87 | Fr3 | D | D | D | D | D | D | D | D | D | D | D | D | D |
| 83 | 88 | Fr3 | V | V | V | V | V | V | V | V | V | V | V | V | V |
| 84 | 89 | Fr3 | G | G | G | G | G | G | G | G | G | G | G | G | G |
| 85 | 90 | Fr3 | V | V | V | V | V | V | V | V | V | V | V | V | V |
| 86 | 91 | Fr3 | Y | Y | Y | Y | Y | Y | Y | Y | Y | Y | Y | Y | Y |
| 87 | 92 | Fr3 | Y | Y | Y | Y | Y | Y | Y | Y | Y | Y | Y | Y | Y |
| 88 | 93 | Fr3 | C | C | C | C | C | C | C | C | C | C | C | C | C |
| 89 | 94 | CDR- | A | M | M | A | A | A | A | A | A | A | A | A | A |
| | | L3 | | | | | | | | | | | | | |
| 90 | 95 | CDR- | Q | Q | Q | Q | Q | Q | Q | Q | Q | Q | Q | Q | Q |
| | | L3 | | | | | | | | | | | | | |
| 91 | 96 | CDR- | N | A | A | L | N | N | N | N | N | N | N | N | N |
| | | L3 | | | | | | | | | | | | | |
| 92 | 97 | CDR- | L | L | L | L | L | L | L | L | L | L | L | L | L |
| | | L3 | | | | | | | | | | | | | |
| 93 | 98 | CDR- | E | Q | Q | E | E | E | E | E | E | E | E | E | E |
| | | L3 | | | | | | | | | | | | | |
| 94 | 99 | CDR- | L | T | T | L | L | L | L | L | L | L | L | L | L |
| | | L3 | | | | | | | | | | | | | |
| 95 | 100 | CDR- | P | P | P | P | P | P | P | P | P | P | P | P | P |
| | | L3 | | | | | | | | | | | | | |
| 95A | | CDR- | — | — | — | — | — | — | — | — | — | — | — | | |
| | | L3 | | | | | | | | | | | | | |
| 95B | | CDR- | — | — | — | — | — | — | — | — | — | — | — | | |
| | | L3 | | | | | | | | | | | | | |
| 95C | | CDR- | — | — | — | — | — | — | — | — | — | — | — | | |
| | | L3 | | | | | | | | | | | | | |
| 95D | | CDR- | — | — | — | — | — | — | — | — | — | — | | | |
| | | L3 | | | | | | | | | | | | | |
| 95E | | CDR- | — | — | — | — | — | — | — | — | — | — | — | | |
| | | L3 | | | | | | | | | | | | | |
| 95F | | CDR- | — | — | — | — | — | — | — | — | — | — | — | | |
| | | L3 | | | | | | | | | | | | | |
| 96 | 101 | CDR- | L | Y | L | Y | L | L | L | L | L | L | L | L | L |
| | | L3 | | | | | | | | | | | | | |
| 97 | 102 | CDR- | T | T | T | T | T | T | T | T | T | T | T | T | T |
| | | L3 | | | | | | | | | | | | | |
| 98 | 103 | Fr4 | F | F | F | F | F | F | F | F | F | F | F | F | F |
| 99 | 104 | Fr4 | G | G | G | G | G | G | G | G | G | G | G | G | G |
| 100 | 105 | Fr4 | A | Q | G | G | G | Q | Q | Q | Q | Q | Q | Q | Q |
| 101 | 106 | Fr4 | G | G | G | G | G | G | G | G | G | G | G | G | G |
| 102 | 107 | Fr4 | T | T | T | T | T | T | T | T | T | T | T | T | T |
| 103 | 108 | Fr4 | K | K | K | K | K | K | K | K | K | K | K | K | K |
| 104 | 109 | Fr4 | L | L | V | L | L | L | L | L | L | L | L | L | L |
| 105 | 110 | Fr4 | E | E | E | E | E | E | E | E | E | E | E | E | E |
| 106 | 111 | Fr4 | L | I | I | I | I | I | I | I | I | I | I | I | I |
| 106A | | Fr4 | — | — | — | — | — | — | — | — | — | — | — | | |
| 107 | 112 | Fr4 | K | K | K | K | K | K | K | K | K | K | K | K | K |

TABLE 8
V_{H}, V_{L} Backmutations and Other Mutations for Humanized 9F5

| | | Changes from Acceptor Framework |
|---|---|---|
| | | (or CDR) Residues (based on |
| V_{H} or V_{L} Variant | V_{H} or V_{L} Exon Acceptor Sequence | Kabat/Chothia Composite CDRs) |
| Hu9F5VHv1 | GenBank Acc. # AAN16432-VH_huFrwk | None |
| (SEQ ID NO: 15) | (SEQ ID NO: 31) | |
| | PDB ID 2RCS-VH_huFrwk | |
| | (SEQ ID NO: 32) | |
| Hu9F5VHv2 | GenBank Acc. # AAN16432-VH_huFrwk | H1, H17, H20, H69, H75, H93, H94, H109 |
| (SEQ ID NO: 16) | (SEQ ID NO: 31) | |
| | PDB ID 2RCS-VH_huFrwk | |
| | (SEQ ID NO: 32) | |
| | IMGT # IGHV1-69-2*01 | |
| | (SEQ ID NO: 33) | |
| Hu9F5VHv3 | GenBank Acc. # AAN16432-VH_huFrwk | H1, H17, H20, H66, H69, H75, H81, H93, |
| (SEQ ID NO: 17) | (SEQ ID NO: 31) | H94, H109 |
| | PDB ID 2RCS-VH_huFrwk | |
| | (SEQ ID NO: 32) | |
| | IMGT # IGHV1-69-2*01 | |
| | (SEQ ID NO: 33) | |
| Hu9F5VHv4 | GenBank Acc. # AAN16432-VH_huFrwk | H1, H17, H20, H23, H28, H66, H69, H75, |
| (SEQ ID NO: 18) | (SEQ ID NO: 31) | H81, H83, H93, H94, H109 |
| | PDB ID 2RCS-VH_huFrwk | |
| | (SEQ ID NO: 32) | |
| | IMGT # IGHV1-69-2*01 | |
| | (SEQ ID NO: 33) | |
| Hu9F5VHv5 | GenBank Acc. # AAN16432-VH_huFrwk | H1, H17, H20, H23, H28, H43, H51, H54, |
| (SEQ ID NO: 19) | (SEQ ID NO: 31) | H56, H66, H69, H75, H76, H80, H81, H83, |
| | PDB ID 2RCS-VH_huFrwk | H93, H94, H109, H109 |
| | (SEQ ID NO: 32) | |
| | IMGT # IGHV1-69-2*01 | |
| | (SEQ ID NO: 33) | |
| Hu9F5VHv6 | GenBank Acc. # AAN16432-VH_huFrwk | H1, H17, H20, H23, H28, H40, H43, H48, |
| (SEQ ID NO: 20) | (SEQ ID NO: 31) | H51, H54, H56, H66, H69, H75, H76, H80, |
| | PDB ID 2RCS-VH_huFrwk | H81, H83, H93, H94, H108, H109 |
| | (SEQ ID NO: 32) | |
| | IMGT # IGHV1-69-2*01 | |
| | (SEQ ID NO: 33) | |
| Hu9F5VHv7 | GenBank Acc. # AAN16432-VH_huFrwk | H1, H5, H11, H12, H17, H20, H23, H38, |
| (SEQ ID NO: 21) | (SEQ ID NO: 31) | H40, H42, H43, H51, H54, H56, H66, H69, |
| | PDB ID 2RCS-VH_huFrwk | H75, H76, H80, H81, H83, H93, H94, H108, |
| | (SEQ ID NO: 32) | H109 |
| | IMGT # IGHV1-69-2*01 | |
| | (SEQ ID NO: 33) | |
| Hu9F5VHv8 | GenBank Acc. # AAN16432-VH_huFrwk | H1, H5, H11, H12, H17, H20, H23, H38, |
| (SEQ ID NO: 22) | (SEQ ID NO: 31) | H40, H42, H43, H51, H66, H69, H75, H76, |
| | PDB ID 2RCS-VH_huFrwk | H80, H81, H83, H93, H94, H108, H109 |
| | (SEQ ID NO: 32) | |
| | IMGT # IGHV1-69-2*01 | |
| | (SEQ ID NO: 33) | |
| Hu9F5VHv9 | GenBank Acc. # AAN16432-VH_huFrwk | H1, H5, H11, H12, H17, H20, H23, H38, |
| (SEQ ID NO: 127) | (SEQ ID NO: 31) | H42, H43, H66, H69, H75, H80, H81, H83, |
| | PDB ID 2RCS-VH_huFrwk | H93, H94, H108, H109 |
| | (SEQ ID NO: 32) | |
| | IMGT # IGHV1-69-2*01 | |
| | (SEQ ID NO: 33) | |
| Hu9F5VHv10 | GenBank Acc. # AAN16432-VH_huFrwk | H1, H5, H11, H12, H17, H20, H23, H43, |
| (SEQ ID NO: 128) | (SEQ ID NO: 31) | H66, H69, H75, H80, H81, H83, H93, H94, |
| | PDB ID 2RCS-VH_huFrwk | H108, H109; |
| | (SEQ ID NO: 32) | |
| | IMGT # IGHV1-69-2*01 | |
| | (SEQ ID NO: 33) | |
| Hu9F5VLv1 | GenBank Acc. # CAB51297-VL_huFrwk | L64, L66 |
| (SEQ ID NO: 23) | (SEQ ID NO: 35) | |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv2 | GenBank Acc. # CAB51297-VL_huFrwk | L7, L8, L15, L64, L66, L100 |
| (SEQ ID NO: 24) | (SEQ ID NO: 35) | |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv3 | GenBank Acc. # CAB51297-VL_huFrwk | L7, L8, L15, L17, L66, L100 |
| (SEQ ID NO: 25) | (SEQ ID NO: 35) | |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv4 | GenBank Acc. # CAB51297-VL_huFrwk | L7, L8, L11, L15, L17, L51, L54, L66, |
| (SEQ ID NO: 26) | (SEQ ID NO: 35) | L100 |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv5 | GenBank Acc. # CAB51297-VL_huFrwk | L7, L8, L11, L15, L17, L30, L51, L54, |
| (SEQ ID NO: 27) | (SEQ ID NO: 35) | L66, L100 |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv6 | GenBank Acc. # CAB51297-VL_huFrwk | L7, L8, L11, L15, L17, L30, L51, L54, |
| (SEQ ID NO: 28) | (SEQ ID NO: 35) | L100 |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv7 | GenBank Acc. # CAB51297-VL_huFrwk | L7, L8, L9, L11, L15, L17, L18, L31, |
| (SEQ ID NO: 29) | (SEQ ID NO: 35) | L39, L51, L54, L60, L66, L74, L100 |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv8 | GenBank Acc. # CAB51297-VL_huFrwk | L7, L8, L11, L15, L17, L39, L64, L66, |
| (SEQ ID NO: 130) | (SEQ ID NO: 35) | L74, L100 |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |
| Hu9F5VLv9 | GenBank Acc. # CAB51297-VL_huFrwk | L7, L8, L11, L15, L17, L39, L60, L64, |
| (SEQ ID NO: 131) | (SEQ ID NO: 35) | L66, L74, L100 |
| | GenBank Acc. # 1911357B-VL_huFrwk | |
| | (SEQ ID NO: 36) | |
| | IMGT # IGKV2-28*01 & IGKJ2*01 | |
| | (SEQ ID NO: 37) | |

TABLE 9
Kabat Numbering of Framework (or CDR) Residues (based on Kabat/Chothia
Composite CDRs) for Backmutations and Other Mutations in Heavy Chains of Humanized
9F5 Antibodies

| | | | IMGT# | | | | | | | |
|---|---|---|---|---|---|---|---|---|---|---|
| | AAN16432- | 2RCS-VH | IGHV1- | Mouse | | | | | | |
| | VH_huFrwk | huFrwk | 69-2*01 | 9F5 VH | Hu9F5VHv1 | Hu9F5VHv2 | Hu9F5VHv3 | Hu9F5VHvv4 | Hu9F5VHvv5 | Hu9F5VHv6 |
| | (SEQ ID | (SEQ ID | (SEQ ID | (SEQ ID | (SEQ ID | (SEQ ID | (SEQ ID | (SEQ ID | (SEQ ID | (SEQ ID |
| Residue | NO: 31) | NO: 32) | NO: 33) | NO: 7) | NO: 15) | NO: 16) | NO: 17) | NO: 18) | NO: 19) | NO: 20) |
| H1 | E | Q | E | E | Q | E | E | E | E | E |
| H5 | V | Q | V | Q | Q | Q | Q | Q | Q | Q |
| H11 | V | L | V | L | L | L | L | L | L | L |
| H12 | K | V | K | V | V | V | V | V | V | V |
| H17 | S | S | T | S | S | T | T | T | T | T |
| H20 | V | L | I | L | L | I | I | I | I | I |
| H23 | K | T | K | T | T | T | T | K | K | K |
| H28 | T | N | T | N | N | N | N | T | T | T |
| H38 | R | K | Q | K | K | K | K | K | K | K |
| H40 | A | R | A | R | R | R | R | R | R | A |
| H42 | G | E | G | E | E | E | E | E | E | E |
| H43 | K | Q | K | R | Q | Q | Q | Q | K | K |
| H48 | M | I | M | I | I | I | I | I | I | M |
| H51 | F | I | V | I | I | I | I | I | V | V |
| H54 | D | N | D | N | N | N | N | N | D | D |
| H56 | E | N | E | D | D | D | D | D | E | E |
| H66 | R | K | R | K | K | K | R | R | R | R |
| H69 | M | I | I | M | I | M | M | M | M | M |
| H75 | T | S | T | S | S | T | T | T | T | T |
| H76 | D | N | D | N | N | N | N | N | D | D |
| H80 | M | L | M | L | L | L | L | L | M | M |
| H81 | E | Q | E | Q | Q | Q | E | E | E | E |
| H83 | R | T | R | T | T | T | T | R | R | R |
| H93 | A | A | A | T | A | T | T | T | T | T |
| H94 | G | S | T | T | S | T | T | T | T | T |
| H108 | L | T | L | L | T | T | T | T | L | L |
| H109 | V | L | V | V | L | V | V | V | V | V |
| | | | | | | | Hu9F5VHv7 | Hu9F5VHv8 | Hu9F5VHv9 | Hu9F5VHv10 |
| | | | | | | | (SEQ ID | (SEQ ID | (SEQ ID | (SEQ ID |
| | | | | | | Residue | NO: 21) | NO: 22) | NO: 127) | NO: 128) |
| | | | | | | H1 | E | E | E | E |
| | | | | | | H5 | V | V | V | V |
| | | | | | | H11 | V | V | V | V |
| | | | | | | H12 | K | K | K | K |
| | | | | | | H17 | T | T | T | T |
| | | | | | | H20 | I | I | I | I |
| | | | | | | H23 | K | K | K | K |
| | | | | | | H28 | N | N | N | N |
| | | | | | | H38 | R | R | Q | K |
| | | | | | | H40 | A | A | R | R |
| | | | | | | H42 | G | G | G | E |
| | | | | | | H43 | K | K | K | K |
| | | | | | | H48 | I | I | I | I |
| | | | | | | H51 | V | V | I | I |
| | | | | | | H54 | D | N | N | N |
| | | | | | | H56 | E | D | D | D |
| | | | | | | H66 | R | R | R | R |
| | | | | | | H69 | M | M | M | M |
| | | | | | | H75 | T | T | T | T |
| | | | | | | H76 | D | D | N | N |
| | | | | | | H80 | M | M | M | M |
| | | | | | | H81 | E | E | E | E |
| | | | | | | H83 | R | R | R | R |
| | | | | | | H93 | T | T | T | T |
| | | | | | | H94 | T | T | T | T |
| | | | | | | H108 | L | L | L | L |
| | | | | | | H109 | V | V | V | V |

TABLE 10
Kabat Numbering of Framework Residues (based on Kabat/Chothia Composite
CDRs) for Backmutations and Other Mutations in Light Chains of Humanized 9F5
Antibodies

| | CAB51297- | 1911357B- | IGKV2-28*01 & | Mouse | | | | | |
|---|---|---|---|---|---|---|---|---|---|
| | VL_huFrwk | VL_huFrwk | IGKJ2*01 | 9F5 VL | Hu9F5VLv1 | Hu9F5VLv2 | Hu9F5VLv3 | Hu9F5VLvv4 | Hu9F5VLv5 |
| | (SEQ ID | (SEQ ID | (SEQ ID | (SEQ ID | (SEQ ID | (SEQ ID | (SEQ ID | (SEQ ID | (SEQ ID |
| Residue | NO: 35) | NO: )36 | NO: 37) | NO: 11) | NO: 23) | NO: 24) | NO: 25) | NO: 26) | NO: 27) |
| L7 | S | A | S | A | A | S | S | S | S |
| L8 | P | A | P | A | A | P | P | P | P |
| L9 | L | F | L | F | F | F | F | F | F |
| L11 | L | N | L | N | N | N | N | L | L |
| L15 | P | L | P | L | L | P | P | P | P |
| L17 | E | T | E | T | T | T | E | E | E |
| L18 | P | S | P | S | S | S | S | S | S |
| L30 | Y | I | Y | I | I | I | I | I | Y |
| L31 | N | T | N | T | T | T | T | T | T |
| L39 | K | R | K | K | R | R | R | R | R |
| L51 | G | V | G | M | M | M | M | G | G |
| L54 | R | L | R | L | L | L | L | R | R |
| L60 | D | N | D | D | N | N | N | N | N |
| L64 | G | G | G | S | S | S | G | G | G |
| L66 | G | E | G | G | G | G | G | G | G |
| L74 | K | R | K | R | R | R | R | R | R |
| L100 | G | G | Q | A | G | Q | Q | Q | Q |
| | | | | | | Hu9F5VLv6 | Hu9F5VLv7 | Hu9F5VLv8 | Hu9F5VLv9 |
| | | | | | | (SEQ ID | (SEQ ID | (SEQ ID | (SEQ ID |
| | | | | | Residue | NO: 28) | NO: 29) | NO: 130) | NO: 131) |
| | | | | | L7 | S | S | S | S |
| | | | | | L8 | P | P | P | P |
| | | | | | L9 | F | L | F | F |
| | | | | | L11 | L | L | L | L |
| | | | | | L15 | P | P | P | P |
| | | | | | L17 | E | E | E | E |
| | | | | | L18 | S | P | S | S |
| | | | | | L30 | Y | I | I | I |
| | | | | | L31 | T | N | T | T |
| | | | | | L39 | R | K | K | K |
| | | | | | L51 | G | G | M | M |
| | | | | | L54 | R | R | L | L |
| | | | | | L60 | N | D | N | D |
| | | | | | L64 | G | G | S | S |
| | | | | | L66 | E | G | G | G |
| | | | | | L74 | R | K | K | K |
| | | | | | L100 | Q | Q | Q | Q |

TABLE 11
Percentage Humanness of Heavy and Light
Chains of Humanized 9F5 Antibodies

| V_{H} or V_{L} Variant | % Humanness |
|---|---|
| Hu9F5VHv1 (SEQ ID NO: 15) | 66.3% |
| Hu9F5VHv2 (SEQ ID NO: 16) | 69.4% |
| Hu9F5VHv3 (SEQ ID NO: 17) | 71.4% |
| Hu9F5VHv4 (SEQ ID NO: 18) | 74.5% |
| Hu9F5VHv5 (SEQ ID NO: 19) | 80.6% |
| Hu9F5VHv6 (SEQ ID NO: 20) | 82.7% |
| Hu9F5VHv7 (SEQ ID NO: 21) | 84.7% |
| Hu9F5VHv8 (SEQ ID NO: 22) | 82.7% |
| Hu9F5VHv9 (SEQ ID NO: 127) | 80.6% |
| Hu9F5VHv10 (SEQ ID NO: 128) | 78.6% |
| Hu9F5VLv1 (SEQ ID NO: 23) | 78.0% |
| Hu9F5VLv2 (SEQ ID NO: 24) | 81.0% |
| Hu9F5VLv3 (SEQ ID NO: 25) | 83.0% |
| Hu9F5VLv4 (SEQ ID NO: 26) | 86.0% |
| Hu9F5VLv5 (SEQ ID NO: 27) | 87.0% |
| Hu9F5VLv6 (SEQ ID NO: 28) | 86.0% |
| Hu9F5VLv7 (SEQ ID NO: 29) | 92.0% |
| Hu9F5VLv8 (SEQ ID NO: 130) | 85% |
| Hu9F5VLv9 (SEQ ID NO: 131) | 86% |

Positions at which canonical, vernier, or interface residues differ between mouse and human acceptor sequences are candidates for substitution. Examples of canonical/CDR interacting residues include Kabat residues H54 and H94 in Table 6. Examples of vernier residues include Kabat residues H28, H48, H69, H93, and H94 in Table 6 and L64 and L66 in Table 7. Examples of interface/packing (VH+VL) residues include Kabat residue H93 in Table 6.

The rationales for selection of the positions indicated in Table 6 in the heavy chain variable region as candidates for substitution are as follows.

Heavy Chain Variable Regions

hu9F5VHv1
consists of the CDR-H1, H2, and H3 loops of 9F5-VH grafted onto the framework of AAN16432_VH & RCS-VH.

hu9F5VHv2
reverts all framework substitutions at positions that are key for defining the Chothia canonical classes, are part of the Vernier zone, or localize to the VH/VL domain interface or contribute to structural stability. hu9F5VHv2 incorporates backmutations or substitutions at various positions as listed below to enable assessment of these positions' contributions to antigen-binding affinity and immunogenicity.

hu9F5VHv3, hu9F5VHv4, hu9F5VHv5, hu9F5VHv6, hu9F5VHv7, hu9F5VHv8, hu9F5VHv9, and hu9F5VHv10,
consists of further substitutions and either to add to Ab stability and/or optimization of deamination, oxidation, N-glycosylation, proteolysis and aggregation

Q1E: is a stability enhancing mutation to mitigate pyroglutamate formation potential to reduce N-term heterogeneity (Liu, supra.)

Q5V: is a germ-line aligning mutation. Val is in human germ line gene IGHV1-69-2*01 (SEQ ID NO:33) at this position.

L11V: is a germ-line aligning mutation. Val is in human germ line gene IGHV1-69-2*01 (SEQ ID NO:33) at this position.

V12K: is a germ-line aligning mutation. Lys is in human germ line gene IGHV1-69-2*01 (SEQ ID NO:33) at this position.

S17T: is a germ-line aligning mutation. Thr is in human germ line gene IGHV1-69-2*01 (SEQ ID NO:33) at this position. Thr substitution may enhance stability over Ser at this position.

L20I: is a germ-line aligning mutation. Ile is in human germ line gene IGHV1-69-2*01 (SEQ ID NO:33) at this position.

T23K: is a frequency-based and germ line-aligning mutation. Lys is in human germ line gene IGHV1-69-2*01 (SEQ ID NO:33) at this position.

N28T: This is a CDR-H1 residue substitution to Thr, and is a germ line-aligning mutation. Thr is in human germ line gene IGHV1-69-2*01 (SEQ ID NO:33) at this position.

K38R: Arg is in AAN16432-VH_huFrwk (SEQ ID NO:31) at this position and may enhance stability over Lys at this position.

K38Q: K38Q is a germ-line aligning mutation. Gln is in human germ line gene IGHV1-69-2*01 (SEQ ID NO:33) at this position. This substitution makes the antibody a more human like sequence and preserves antibody function at the same time.

R40A: is a frequency-based and germ line-aligning mutation. Ala is in human germ line gene IGHV1-69-2*01 (SEQ ID NO:33) at this position.

E42G: is a germ-line aligning mutation. Gly is in human germ line gene IGHV1-69-2*01 (SEQ ID NO:33) at this position.

Q43K: is a frequency-based and germ line-aligning mutation. Lys is in human germ line gene IGHV1-69-2*01 (SEQ ID NO:33) at this position. Lys at this position may enhance stability.

I48M: is a frequency-based and germ line-aligning mutation of a Vernier zone residue. Met is in human germ line gene IGHV1-69-2*01 (SEQ ID NO:33) at this position.

I51V: This is a CDR-H2 residue substitution to Val. This position is predicted to be a non-antigen contact position as per homology model. This is a germ line-aligning mutation. Val is in human germ line gene IGHV1-69-2*01 (SEQ ID NO:33) at this position.

N54D and D56E: These are CDR-H2 residue substitutions and are predicted to be non-antigen contact positions as per homology model. N54D and D56E substitutions are predicted to stabilize antibody structure.

K66R: Arg at this position is predicted to make H-bonds with Ser 82a & Thr 83 in addition to making H-bond and salt-bridge with Asp 86.

I69M is a backmutation of a Vernier zone residue.

S75T: Ser at this position is predicted to make H-bond with Asp 72 and Tyr 76. Thr at this position is predicted to also make these contacts but being surface exposed residue Thr may enhance antibody stability. S75T is germ-line aligning mutation. Thr is in human germ line gene IGHV1-69-2*01 (SEQ ID NO:33) at this position. Thr is in acceptor sequence AAN16432-VH_huFrwk at this position.

N76D: is a germ-line aligning mutation. Asp is in human germ line gene IGHV1-69-2*01 (SEQ ID NO:33) at this position. Asp at this position may reduce N-glycosylation potential.

L80M: is a germ-line aligning mutation. Met is in human germ line gene IGHV1-69-2*01 (SEQ ID NO:33) at this position.

Q81E: Glu is predicted to make H-bond plus salt-bridge with Lys19 hence Glu at this position make enhance antibody stability.

T83R: is a frequency-based and germ line-aligning mutation. Arg is in human germ line gene IGHV1-69-2*01 (SEQ ID NO:33) at this position.

A93T: Position H93 is a heavy chain/light chain interface residue. Backmutations to murine residue Thr at this position preserves this interface. A93T is also a germ-line-aligning mutation. Thr is in human germ line gene IGHV1-69-2*01 (SEQ ID NO:33) at this position. A93T at this position makes antibody more human-like.

S94T: is a back-mutation of a Chothia defined canonical structural residue and vernier residue. S94T is also a germ-line-aligning mutation. Thr is in human germ line gene IGHV1-69-2*01 (SEQ ID NO:33) at this position.

T108L: is a germ-line aligning mutation. Leu is in human germ line gene IGHV1-69-2*01 (SEQ ID NO:33) at this position.

L109V: is a frequency-based and germ line-aligning mutation. Val is most frequent at this position. Val is in human germ line gene IGHV1-69-2*01 (SEQ ID NO:33) at this position.

The rationales for selection of the positions indicated in Table 7 in the light chain variable region as candidates for substitution are as follows.

Kappa Light Chain Variable Regions

hu9F5VLv1
consists of the CDR-L1, L2, and L3 loops of 9F5-VL grafted onto the framework of CAB51297 VL & 1911357B VL along with reverting all framework substitutions at positions that are key for defining the Chothia canonical classes, are part of the Vernier zone, or locate to the VH/VL domain interface.

hu9F5VLv2, hu9F5VLv3, hu9F5VLv4, hu9F5VLv5, hu9F5VLv6, hu9F5VLv7, hu9F5VLv8, and hu9F5VLv9 also include substitution that contribute to structural stability antigen-binding affinity and immunogenicity. Listed below are the substitutions made in these versions.

A7S: is a germ-line aligning mutation. Ser is in human germ line gene IGKV2-28*01 & IGKJ2*01 (SEQ ID NO:37) at this position. Ser substitution may enhance stability as Ser side chain makes H-bond with main chain of Phe9 thereby making the loop more stable.

ABP: is a frequency-based and germ line-aligning mutation. Pro is in human germ line gene IGKV2-28*01 & IGKJ2*01 (SEQ ID NO:37) at this position.

F9L: is a germ-line aligning mutation. Leu is in human germ line gene IGKV2-28*01 & IGKJ2*01 (SEQ ID NO:37) at this position.

N11L: is a frequency-based and germ line-aligning mutation. Leu is in human germ line gene IGKV2-28*01 & IGKJ2*01 (SEQ ID NO:37) at this position.

L15P: is a frequency-based and germ line-aligning mutation. Pro is in human germ line gene IGKV2-28*01 & IGKJ2*01 (SEQ ID NO:37) at this position. Pro at this position may fit better to support the protein stand turn at this position.

T17E: is a frequency-based mutation. Glu is frequent at this position, and Glu at this position is predicted to make H-bond with T14 and salt-bridge with Lys 107, both light chain residues, and to enhance antibody stability.

S18P: is a germ-line aligning mutation. Pro is in human germ line gene IGKV2-28*01 & IGKJ2*01 (SEQ ID NO:37) at this position.

I30Y: This is a substitution of a CDR-L1 residue predicted to be in the antigen contact zone. Tyr at this position makes pie stack with Tyr32 and His27D thus enhancing stability. Since Ile and Tyr are both hydrophobic, Tyr substitution may be well tolerated.

T31N: This is a substitution of a CDR-L1 residue. Asn is in human germ line gene IGKV2-28*01 & IGKJ2*01 (SEQ ID NO:37) at this position.

R39K: is a germ-line aligning mutation. Lys is in human germ line gene IGKV2-28*01 & IGKJ2*01 (SEQ ID NO:37) at this position. Lys may provide equivalent structural support as Arg.

M51G: This is a substitution of a CDR-L2 residue and is predicted to face away from the antigen interface. Gly is also a germ line-aligning mutation. Gly is in human germ line gene IGKV2-28*01 & IGKJ2*01 (SEQ ID NO:37) at this position. This position is predicted to be surface-exposed. Gly at this position, being neutral, reduces potential for Met oxidation.

L54R: This is a substitution of a CDR-L2 residue and is predicted to face away from the antigen interface. Arg is also a germ line-aligning mutation. Arg is in human germ line gene IGKV2-28*01 & IGKJ2*01 (SEQ ID NO:37) at this position. Arg is predicted to make H-bond each with Asn60 and Phe62 chains; increasing stability of inter chain-loop.

N60D: is a germ-line aligning mutation. Asp is in human germ line gene IGKV2-28*01 & IGKJ2*01 (SEQ ID NO:37) at this position.

G64S: is a back-mutation of a Vernier zone residue. G64S back-mutation preserves the CDR conformation.

E66G: is a back-mutation of a Vernier zone residue. E66G back-mutation preserves the CDR conformation. E66G is a germ-line-aligning mutation. Gly is in human germ line gene IGKV2-28*01 & IGKJ2*01 (SEQ ID NO:37) at this position.

R74K: is a germ-line aligning mutation. Lys is in human germ line gene IGKV2-28*01 & IGKJ2*01 (SEQ ID NO:37) at this position.

G100Q: is a frequency-based and germ line-aligning mutation. Gln is in human germ line gene IGKV2-28*01 & IGKJ2*01 (SEQ ID NO:37) at this position. This residue is predicted to be surface-exposed, and therefore, Gln at this position may be better for antibody solubility since it is hydrophilic neutral. Gln at this position may enhance stability.

The designs based on these human frameworks were:

heavy chain variable regions
> hu9F5VHv1
(SEQ ID NO: 15)
QVQLQQSGAELVKPGASVKLSCTASGFNIKDDYMNWVKQRPEQGLEWIGW
IDPENGDTEYASKFQGKATITADTSSNTAYLQLSSLTSEDTAVYYCASSN
GWGQGTTLTVSS
> hu9F5VHv2
(SEQ ID NO: 16)
EVQLQQSGAELVKPGATVKISCTASGFNIKDDYMNWVKQRPEQGLEWIGW
IDPENGDTEYASKFQGKATMTADTSTNTAYLQLSSLTSEDTAVYYCTTSN
GWGQGTTVTVSS
> hu9F5VHv3
(SEQ ID NO: 17)
EVQLQQSGAELVKPGATVKISCTASGFNIKDDYMNWVKQRPEQGLEWIGW
IDPENGDTEYASKFQGRATMTADTSTNTAYLELSSLTSEDTAVYYCTTSN
GWGQGTTVTVSS
> hu9F5VHv4
(SEQ ID NO: 18)
EVQLQQSGAELVKPGATVKISCKASGFTIKDDYMNWVKQRPEQGLEWIGW
IDPENGDTEYASKFQGRATMTADTSTNTAYLELSSLRSEDTAVYYCTTSN
GWGQGTTVTVSS
> hu9F5VHv5
(SEQ ID NO: 19)
EVQLQQSGAELVKPGATVKISCKASGFTIKDDYMNWVKQRPEKGLEWIGW
VDPEDGETEYASKFQGRATMTADTSTDTAYMELSSLRSEDTAVYYCTTSN
GWGQGTLVTVSS
> hu9F5VHv6
(SEQ ID NO: 20)
EVQLQQSGAELVKPGATVKISCKASGFTIKDDYMNWVKQAPEKGLEWMGW
VDPEDGETEYASKFQGRATMTADTSTDTAYMELSSLRSEDTAVYYCTTSN
GWGQGTLVTVSS
> hu9F5VHv7
(SEQ ID NO: 21)
EVQLVQSGAEVKKPGATVKISCKASGFNIKDDYMNWVRQAPGKGLEWIGW
VDPEDGETEYASKFQGRATMTADTSTDTAYMELSSLRSEDTAVYYCTTSN
GWGQGTLVTVSS
> hu9F5VHv8
(SEQ ID NO: 22)
EVQLVQSGAEVKKPGATVKISCKASGFNIKDDYMNWVRQAPGKGLEWIGW
VDPENGDTEYASKFQGRATMTADTSTDTAYMELSSLRSEDTAVYYCTTSN
GWGQGTLVTVSS
> hu9F5VHv9
(SEQ ID NO: 127)
EVQLVQSGAEVKKPGATVKISCKASGFNIKDDYMNWVQQRPGKGLEWIGW
IDPENGDTEYASKFQGRATMTADTSTNTAYMELSSLRSEDTAVYYCTTSN
GWGQGTLVTVSS
> hu9F5VHv10, (also known as hu9F5VHv9_Q38K_G42E)
(SEQ ID NO: 128)
EVQLVQSGAEVKKPGATVKISCKASGFNIKDDYMNWVKQRPEKGLEWIGW
IDPENGDTEYASKFQGRATMTADTSTNTAYMELSSLRSEDTAVYYCTTSN
GWGQGTLVTVSS
kappa light chain variable regions
> hu9F5VLv1
(SEQ ID NO: 23)
DIVMTQAAFSNPVTLGTSASISCRSSKSLLHSNGITYLYWYLQRPGQSPQ
LLIYQMSNLASGVPNRFSSSGSGTDFTLRISRVEAEDVGVYYCAQNLELP
LTFGGGTKLEIK
> hu9F5VLv2
(SEQ ID NO: 24)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNLELP
LTFGQGTKLEIK
> hu9F5VLv3
(SEQ ID NO: 25)
DIVMTQSPFSNPVTPGESASISCRSSKSLLHSNGITYLYWYLQRPGQSPQ
LLIYQMSNLASGVPNRFSGSGSGTDFTLRISRVEAEDVGVYYCAQNLELP
LTFGQGTKLEIK
> hu9F5VLv4
(SEQ ID NO: 26)
DIVMTQSPFSLPVTPGESASISCRSSKSLLHSNGITYLYWYLQRPGQSPQ
LLIYQGSNRASGVPNRFSGSGSGTDFTLRISRVEAEDVGVYYCAQNLELP
LTFGQGTKLEIK
> hu9F5VLv5
(SEQ ID NO: 27)
DIVMTQSPFSLPVTPGESASISCRSSKSLLHSNGYTYLYWYLQRPGQSPQ
LLIYQGSNRASGVPNRFSGSGSGTDFTLRISRVEAEDVGVYYCAQNLELP
LTFGQGTKLEIK
> hu9F5VLv6
(SEQ ID NO: 28)
DIVMTQSPFSLPVTPGESASISCRSSKSLLHSNGYTYLYWYLQRPGQSPQ
LLIYQGSNRASGVPNRFSGSESGTDFTLRISRVEAEDVGVYYCAQNLELP
LTFGQGTKLEIK
> hu9F5VLv7
(SEQ ID NO: 29)
DIVMTQSPLSLPVTPGEPASISCRSSKSLLHSNGINYLYWYLQKPGQSPQ
LLIYQGSNRASGVPDRFSGSGSGTDFTLKISRVEAEDVGVYYCAQNLELP
LTFGQGTKLEIK
> hu9F5VLv8
(SEQ ID NO: 130)
DIVMTQSPFSLPVTPGESASISCRSSKSLLHSNGITYLYWYLQKPGQSPQ
LLIYQMSNLASGVPNRFSSSGSGTDFTLKISRVEAEDVGVYYCAQNLELP
LTFGQGTKLEIK
> hu9F5VLv9, also known as hu9F5VLv8_N60D
(SEQ ID NO: 131)
DIVMTQSPFSLPVTPGESASISCRSSKSLLHSNGITYLYWYLQKPGQSPQ
LLIYQMSNLASGVPDRFSSSGSGTDFTLKISRVEAEDVGVYYCAQNLELP
LTFGQGTKLEIK

Humanized sequences are generated using a two-stage PCR protocol that allows introduction of multiple mutations, deletions, and insertions using QuikChange site-directed mutagenesis [Wang, W. and Malcolm, B. A. (1999) BioTechniques 26:680-682).

### Example 5. Immunogenicity of hu9F5VHv4/hu9F5VLv2

The amino acid sequences of the heavy chain variable region (SEQ ID NO:18) and light chain variable region (SEQ ID NO:24) of hu9F5VHv4/hu9F5VLv2 were analyzed using iedb.org Deimmunization Tool (Dhanda et al, Immunology. 2018 January; 153(1):118-132). Table 12 shows the peptides that may be selected for deimmunization of hu9F5VLv2, i.e., suggesting areas where further substitutions could be made to reduce potential immunogenicity.

TABLE 12
Results of immunogenicity analysis identifying
potential peptides within hu9F5VLv2 for
deimmunization

| Start | Stop | Median | |
|---|---|---|---|
| position | position | percentile | |
| (linear) | (linear) | rank | Peptide |
| 51 | 65 | 9.895 | LLIYQMSNLASGVPN |
| 46 | 60 | 15.18 | GQSPQLLIYQMSNLA |
| 26 | 40 | 16.055 | SKSLLHSNGITYLW |
| 76 | 90 | 18.23 | FTLRISRVEAEDVGV |
| 36 | 50 | 19.455 | TYLYWYLQRPGQSPQ |
| 86 | 100 | 19.535 | EDVGVYYCAQNLELP |

Based on the results of the analysis shown in Table 12, variants of hu9F5VLv2 light chain variable region were designed, targeting the amino acid residues bolded in Table 13. Each variant incorporates one of the following amino acid substitutions, as shown in Table 14.

TABLE 13
Amino acid residues targeted for substitution.

| | Original |
|---|---|
| Targeted residues of hu9F5VLv2 | SEQ ID |
| light chain variable region | NO |
| DIVMTQSPFSNPVTPGTSASISCRSSKSLL | 24 |
| HSNGITYLYWYLQRPGQSPQLLIYQMSNLA | |
| SGVPNRFSSSGSGTDFTLRISRVEAEDVGV | |
| YYCAQNLELPLTFGQGTKLEIK | |

TABLE 14
Amino acid substitutions made in hu9F5VLv2 light
chain variable region to reduce immunogenicity

| | | SEQ ID NO of |
|---|---|---|
| Linear | Kabat | resulting light chain |
| Number | Number | variable region |
| M56E | M51E | SEQ ID NO: 61 |
| M56D | M51D | SEQ ID NO: 62 |
| L30D | L27cD | SEQ ID NO: 63 |
| L30G | L27cG | SEQ ID NO: 64 |
| L30S | L27cS | SEQ ID NO: 65 |
| L30E | L27cE | SEQ ID NO: 66 |
| I35E | I30E | SEQ ID NO: 67 |
| I35K | I30K | SEQ ID NO: 68 |
| L30T | L27cT | SEQ ID NO: 69 |
| L30N | L27cN | SEQ ID NO: 70 |
| L29D | L27bD | SEQ ID NO: 71 |
| I35G | I30G | SEQ ID NO: 72 |
| L38N | L33N | SEQ ID NO: 73 |
| L30A | L27cA | SEQ ID NO: 74 |
| L38T | L33T | SEQ ID NO: 75 |
| L38S | L33S | SEQ ID NO: 76 |
| L38R | L33R | SEQ ID NO: 77 |
| I35Q | I30Q | SEQ ID NO: 78 |
| L29T | L27bT | SEQ ID NO: 79 |
| T36G | T31G | SEQ ID NO: 80 |
| L29Q | L27bQ | SEQ ID NO: 81 |
| L38G | L33G | SEQ ID NO: 82 |
| L30P | L27cP | SEQ ID NO: 83 |
| V83R | V78R | SEQ ID NO: 84 |
| I80D | I75D | SEQ ID NO: 85 |
| V83D | V78D | SEQ ID NO: 86 |
| V83E | V78E | SEQ ID NO: 87 |
| V83P | V78P | SEQ ID NO: 88 |
| V83K | V78K | SEQ ID NO: 89 |
| R82D | R77D | SEQ ID NO: 90 |
| V83G | V78G | SEQ ID NO: 91 |
| S81P | S76P | SEQ ID NO: 92 |
| I80P | I75P | SEQ ID NO: 93 |
| I80Q | I75Q | SEQ ID NO: 94 |
| I80G | I75G | SEQ ID NO: 95 |
| L78P | L73P | SEQ ID NO: 96 |
| L78G | L73G | SEQ ID NO: 97 |
| V83Q | V78Q | SEQ ID NO: 98 |
| S81G | S76G | SEQ ID NO: 99 |
| L97D | L92D | SEQ ID NO: 100 |
| Y91T | Y86T | SEQ ID NO: 101 |
| L97E | L92E | SEQ ID NO: 102 |
| L97G | L92G | SEQ ID NO: 103 |
| L97Q | L92Q | SEQ ID NO: 104 |
| L99G | L93G | SEQ ID NO: 105 |
| V90G | V85G | SEQ ID NO: 106 |
| L97T | L92T | SEQ ID NO: 107 |
| A94G | A89G | SEQ ID NO: 108 |

Similarly, hu9F5VHv4 was analyzed for potential immunogenicity and Table 15 shows the peptide identified for potential deimmunization.

TABLE 15
Results of immunogenicity analysis identifying
potential peptide within hu9F5VHv4 for
deimmunization

| Start | Stop | Median | |
|---|---|---|---|
| position | position | percentile | |
| (linear) | (linear) | rank | Peptide |
| 76 | 90 | 11.925 | TNTAYLELSSLRSED |

Based on these results, variants of hu9F5VHv4 heavy chain variable region and hu9F5VHv5 heavy chain variable region were designed, targeting the amino acid residues bolded in Table 16. Each variant incorporates one of the following amino acid substitutions, as shown in Table 17.

TABLE 16
Amino acid residues targeted for substitution.

| | Original |
|---|---|
| Targeted residues of heavy chain | SEQ ID |
| variable region | NO |
| hu9F5VHv4 | 18 |
| EVQLQQSGAELVKPGATVKISCKASGFTIK | |
| DDYMNWVKQRPEQGLEWIGWIDPENGDTEY | |
| ASKFQGRATMTADTSTNTAYLELSSLRSED | |
| TAVYYCTTSNGWGQGTTVTVSS | |
| hu9F5VHv5 | 19 |
| EVQLQQSGAELVKPGATVKISCKASGFTIK | |
| DDYMNWVKQRPEKGLEWIGWVDPEDGETEY | |
| ASKFQGRATMTADTSTDTAYMELSSLRSED | |
| TAVYYCTTSNGWGQGTLVTVSS | |

TABLE 17
Amino acid substitutions made in hu9F5VHv4 or v5 as indicated
heavy chain variable region to reduce immunogenicity

| | | 9F5 variant in | SEQ ID NO of |
|---|---|---|---|
| Linear | Kabat | which substitutions | resulting heavy chain |
| Number | Number | are made | variable region |
| L81P | L80P | hu9F5VHv4 | SEQ ID NO: 109 |
| L81D | L80D | hu9F5VHv4 | SEQ ID NO: 110 |
| L86G | L82cG | hu9F5VHv4 | SEQ ID NO: 111 |
| L86D | L82cD | hu9F5VHv4 | SEQ ID NO: 112 |
| L83P | L82P | hu9F5VHv4 | SEQ ID NO: 113 |
| L81G | L80G | hu9F5VHv4 | SEQ ID NO: 114 |
| L83K | L82K | hu9F5VHv4 | SEQ ID NO: 115 |
| L83R | L82R | hu9F5VHv4 | SEQ ID NO: 116 |
| L83E | L82E | hu9F5VHv4 | SEQ ID NO: 117 |
| L83N | L82N | hu9F5VHv4 | SEQ ID NO: 118 |
| Y80D | Y79D | hu9F5VHv4 | SEQ ID NO: 119 |
| Y80N | Y79N | hu9F5VHv4 | SEQ ID NO: 120 |
| Y80G | Y79G | hu9F5VHv4 | SEQ ID NO: 121 |
| M81E | M80E | hu9F5VHv5 | SEQ ID NO: 122 |
| M81G | M80G | hu9F5VHv5 | SEQ ID NO: 123 |
| L86S | L82cS | hu9F5VHv4 | SEQ ID NO: 124 |
| Y80Q | Y79Q | hu9F5VHv4 | SEQ ID NO: 125 |
| S84G | S82aG | hu9F5VHv4 | SEQ ID NO: 126 |

### Example 6 Immunogenicity of hu9F5VLv8 and hu9F5VHv10

The amino acid sequences of the heavy chain variable region (SEQ ID NO:128) and light chain variable region (SEQ ID NO:130) of hu9F5VHv10/hu9F5VLv8 were analyzed using iedb.org Deimmunization Tool (Dhanda et al, Immunology. 2018 January; 153(1):118-132). Table 18 shows the peptides that may be selected for deimmunization of hu9F5VLv8, i.e., suggesting areas where further substitutions could be made to reduce potential immunogenicity.

TABLE 18
Results of immunogenicity analysis identifying
potential peptides within hu9F5VLv8 for
deimmunization

| Start | Stop | Median | |
|---|---|---|---|
| position | position | percentile | |
| (linear) | (linear) | rank | Peptide |
| 51 | 65 | 9.895 | LLIYQMSNLASGVPN |
| 46 | 60 | 15.18 | GQSPQLLIYQMSNLA |
| 26 | 40 | 16.055 | SKSLLHSNGITYLYW |
| 1 | 15 | 17.995 | DIVMTQSPFSLPVTP |
| 86 | 100 | 19.535 | EDVGVYYCAQNLELP |
| 36 | 50 | 19.84 | TYLWYLQKPGQSPQ |

Based on the results of the analysis shown in Table 19, variants of hu9F5VLv8 light chain variable region were designed, targeting the amino acid residues bolded in Table 19. Each variant incorporates one or more amino acid substitutions at the bolded residues of Table 19 as shown in Tables 20-21. Hu9F5VLv8 with substitution N60D is also known as hu9F5VLv9.

TABLE 19
Amino acid residues targeted for substitution.

| | SEQ ID |
|---|---|
| Targeted residues of hu9F5VLv8 light | NO: of |
| chain variable region | hu9F5VLv8 |
| DIVMTQSPFSLPVTPGESASISCRSSKSLL | 130 |
| HSNGITYLYWYLQKPGQSPQLLIYQMSNLA | |
| SGVPNRFSSSGSGTDFTLKISRVEAEDVGV | |
| YYCAQNLELPLTFGQGTKLEIK | |

TABLE 20
Amino acid substitutions made in hu9F5VLv8 light
chain variable region to reduce immunogenicity

| | Substitutions in Additional | |
|---|---|---|
| | Variants in hu9F5VLv8 designed | SEQ ID NO: of |
| | to reduce immunogenicity (Kabat | resulting light chain |
| Variant Name | number of mutation(s) indicated) | variable region |
| hu9F5VLv8_DIM1 | V3Q, L27cS, L37Q, M51G, L54G, L92I | SEQ ID NO: 132 |
| hu9F5VLv8_DIM2 | V3Q, L27cS, L37Q, M51G, L54R, L92I | SEQ ID NO: 133 |
| hu9F5VLv8_DIM3 | V3Q, L27cS, L37Q, M51G, L54T, L92I | SEQ ID NO: 134 |
| hu9F5VLv8_DIM4 | V3Q, L27cS, L37Q, M51G, L54R, L92G | SEQ ID NO: 135 |
| hu9F5VLv8_DIM5 | V3Q, L27cG, L37Q, M51G, L54R, L92I | SEQ ID NO: 136 |
| hu9F5VLv8_DIM6 | V3Q, L27cD, L37Q, M51G, L54R, L92I | SEQ ID NO: 137 |
| hu9F5VLv8_DIM7 | V3Q, L27cD, L37Q, M51K, L54R, L92I | SEQ ID NO: 138 |
| hu9F5VLv8_DIM8 | V3Q, L27cG, L37Q, M51K, L54R, L92I | SEQ ID NO: 139 |
| hu9F5VLv8_DIM9 | V3Q, L27cG, L37Q, M51K, L54G, L92I | SEQ ID NO: 140 |
| hu9F5VLv8_DIM10 | V3Q, L27cS, L37Q, M51K, L54G, L92I | SEQ ID NO: 141 |
| hu9F5VLv8_DIM11 | V3Q, L27cG, L37G, M51G, L54R, L92I | SEQ ID NO: 142 |
| hu9F5VLv8_DIM12 | V3Q, L27cG, L37G, M51G, L54R, L92G | SEQ ID NO: 143 |
| hu9F5VLv8_DIM13 | V3Q, L27cG, L37G, M51G, L54R | SEQ ID NO: 144 |
| hu9F5VLv8_DIM14 | V3Q, L27cG, L37G, M51G, L54T, L92I | SEQ ID NO: 145 |
| hu9F5VLv8_DIM15 | V3Q, L27cG, L37G, M51G, L54T, L92G | SEQ ID NO: 146 |
| hu9F5VLv8_DIM16 | V3Q, L27cG, L37G, M51G, L54T | SEQ ID NO: 147 |
| hu9F5VLv8_DIM17 | V3Q, L27cS, L37G, M51G, L54T, L92I | SEQ ID NO: 148 |
| hu9F5VLv8_DIM18 | V3Q, L27cD, L37G, M51G, L54R, L92I | SEQ ID NO: 149 |
| hu9F5VLv8_DIM19 | V3Q, L27cS, L37I, M51I, L54R, L92I | SEQ ID NO: 150 |
| hu9F5VLv8_DIM20 | V3Q, L27cS, L37Q, M51I, L54G, L92I | SEQ ID NO: 151 |
| hu9F5VLv8_DIM21 | V3Q, L27cS, L37Q, M51I, L54G | SEQ ID NO: 152 |
| hu9F5VLv8_DIM22 | V3Q, L27cS, L37Q, M51E, L54R, L92I | SEQ ID NO: 153 |
| hu9F5VLv8_DIM23 | V3Q, L27cG, L37Q, M51E, L54G, L92I | SEQ ID NO: 154 |
| hu9F5VLv8_DIM24 | V3Q, L27cG, L37I, M51E, L54R, L92I | SEQ ID NO: 155 |
| hu9F5VLv8_DIM25 | V3Q, L27cG, L37I, M51E, L54R, L92G | SEQ ID NO: 156 |
| hu9F5VLv8_DIM26 | V3Q, L27cI, L37I, M51E, L54R | SEQ ID NO: 157 |
| hu9F5VLv8_DIM27 | V3Q, L37Q, M51G, L54R, L92I | SEQ ID NO: 158 |
| hu9F5VLv8_DIM28 | V3Q, L27cS, M51G, L54R, L92I | SEQ ID NO: 159 |
| hu9F5VLv8_DIM29 | V3Q, L27cS, L37Q, L54R, L92I | SEQ ID NO: 160 |
| hu9F5VLv8_DIM30 | V3Q, L27cS, L37Q, M51G, L92I | SEQ ID NO: 161 |

TABLE 21
Amino acid substitutions made in hu9F5VLv9 light
chain variable region to reduce immunogenicity

| | Substitutions in Additional | |
|---|---|---|
| | Variants in hu9F5VLv9 designed | SEQ ID NO: of |
| | to reduce immunogenicity (Kabat | resulting light chain |
| Variant Name | number of mutation(s) indicated) | variable region |
| hu9F5VLv9_DIM1 | V3Q, L27cS, L37Q, M51G, L54G, L92I | SEQ ID NO: 162 |
| hu9F5VLv9_DIM2 | V3Q, L27cS, L37Q, M51G, L54R, L92I | SEQ ID NO: 163 |
| hu9F5VLv9_DIM4 | V3Q, L27cS, L37Q, M51G, L54R, L92G | SEQ ID NO: 164 |
| hu9F5VLv9_DIM5 | V3Q, L27cG, L37Q, M51G, L54R, L92I | SEQ ID NO: 165 |
| hu9F5VLv9_DIM8 | V3Q, L27cG, L37Q, M51K, L54R, L92I | SEQ ID NO: 166 |
| hu9F5VLv9_DIM10 | V3Q, L27cS, L37Q, M51K, L54G, L92I | SEQ ID NO: 167 |
| hu9F5VLv9_DIM11 | V3Q, L27cG, L37G, M51G, L54R, L92I | SEQ ID NO: 168 |
| hu9F5VLv9_DIM13 | V3Q, L27cG, L37G, M51G, L54R | SEQ ID NO: 169 |
| hu9F5VLv9_DIM19 | V3Q, L27cS, L37I, M51I, L54R, L92I | SEQ ID NO: 170 |
| hu9F5VLv9_DIM20 | V3Q, L27cS, L37Q, M51I, L54G, L92I | SEQ ID NO: 171 |

The rationales for selection of the positions as indicated in Table 20 and Table 21 in the light chain variable region as candidates for substitution are as follows.

Mutation at V3Q was designed based on guidance from deimmunization analysis. Gln at position 3 is the second most frequent at this position in the human sequence, therefore, V3Q substitution is designed to reduce immunogenicity and keep the sequence more human-like. Immunogenicity prediction for hu9F5VLv8_V3Q substitutions shows elimination of the N-term 1-15 peptide.

The following mutations were designed based on guidance from deimmunization analysis and also taking into consideration position of the given residue in the homology model. The mutations were also designed to preserve antibody functionality by conserving size and charge/polarity of the substituted residue. Priority was given to a residue if it happens to be the same residue in human germline.

L27cI, L27cS, L27cD, L27cG: to reduce immunogenicity

L37Q, L37G, L37I: to reduce immunogenicity

M51G, M51K, M51I, M51E: to reduce immunogenicity

M51G: This is a substitution of a CDR-L2 residue and is predicted to face away from the antigen interface. Gly is also a germ line-aligning mutation. Gly is in human germ line gene IGKV2-28*01 & IGKJ2*01 (SEQ ID NO:37) at this position. This position is predicted to be surface-exposed. Gly at this position, being neutral, reduces potential for Met oxidation.

L54G, L54R, L54T; to reduce immunogenicity

L54R: This is a substitution of a CDR-L2 residue and is predicted to face away from the antigen interface. Arg is also a germ line-aligning mutation. Arg is in human germ line gene IGKV2-28*01 & IGKJ2*01 (SEQ ID NO:37) at this position. Arg is predicted to make H-bond each with Asn60 and Phe62 chains; increasing stability of inter chain-loop.

N60D: to reduce immunogenicity. N60D: is also a germ-line aligning mutation. Asp is in human germ line gene IGKV2-28*01 & IGKJ2*01 (SEQ ID NO:37) at this position.

L92I, L92G: to reduce immunogenicity

TABLE 22
Results of immunogenicity analysis identifying
potential peptides within hu9F5VHv10 for
deimmunization

| Start | Stop | Median | |
|---|---|---|---|
| position | position | percentile | |
| (linear) | (linear) | rank | Peptide |
| 76 | 90 | 12.72 | TNTAYMELSSLRSED |

Table 22 shows the peptides that may be selected for deimmunization of hu9F5VHv10, i.e., suggesting areas where further substitutions could be made to reduce potential immunogenicity.

Based on these results, a variant of hu9F5VHv10 heavy chain variable region was designed, targeting amino acid residue H82c bolded in Table 23. Variant hu9F5VHv10_L82cG incorporates substitution L82cG, as shown in Table 24.

TABLE 23
Amino acid residues targeted for substitution.

| | Original |
|---|---|
| Targeted residues of heavy chain | SEQ ID |
| variable region | NO |
| >hu9F5VHv10 | 128 |
| EVQLVQSGAEVKKPGATVKISCKASGFNIKDDY | |
| MNWVKQRPEKGLEWIGWIDPENGDTEYASKFQG | |
| RATMTADTSTNTAYMELSSLRSEDTAVYYCTTS | |
| NGWGQGTLVTVSS | |

TABLE 24
Amino acid substitution made in hu9F5VHv10 heavy
chain variable region to reduce immunogenicity

| | Substitutions in Additional | |
|---|---|---|
| | Variants in hu9F5VHv10 designed | SEQ ID NO: of |
| | to reduce immunogenicity (Kabat | resulting heavy chain |
| Variant Name | number of mutation(s) indicated) | variable region |
| hu9F5VHv10_L82cG | L82cG | SEQ ID NO: 129 |

### Example 7 Analysis of Humanized 9F5 Variants

Humanized 9F5 variants with predicted deimmunizing substitutions were analyzed for several characteristics, including target binding affinity, activity in cell-based assays, thermostability, expression characteristics, and number of substitutions. In all cases, results were compared with the parental sequence, hu9F5VHv9/hu9F5VLv8, to determine if any loss of activity or stability was noted.

Target binding analysis was performed using a Biacore T200 to compare the binding affinity of humanized 9F5 variants to recombinant human 4R0N tau. Anti-Human Fc antibody was immobilized on sensor chip CM3 via amine coupling, and humanized 9F5 variants were captured to equivalent levels. Various concentrations of recombinant 4R0N human tau (ranging from 0.02 nM to 12.5 nM) were passed over the captured ligand at 50 μL/min in running buffer (HBS+0.05% P-20, 1 mg/mL BSA) for 180 seconds association/420 seconds dissociation as a single cycle. Data was blank subtracted to both an irrelevant sensor not containing antibody and 0 nM analyte concentration. Analysis was performed using a global 1:1 fit with Biacore Evaluation software.

Affinity determination revealed a number of deimmunized variants that retained the affinity of the parental, which was determined by comparison of the K_{D} of each antibody. In this case, K_{D} of antibodies were determined to be comparable within 3-fold of hu9F5VHv9/hu9F5VLv8 (Table 25) include hu9F5VHv9/hu9F5VLv8_DIM2, hu9F5VHv9/hu9F5VLv8_DIM5, hu9F5VHv9/hu9F5VLv8_DIM6, hu9F5VHv9/hu9F5VLv8_DIM7, hu9F5VHv9/hu9F5VLv8_DIM8, hu9F5VHv9/hu9F5VLv8_DIM11, hu9F5VHv9/hu9F5VLv8_DIM12, hu9F5VHv9/hu9F5VLv8_DIM13, hu9F5VHv9/hu9F5VLv8_DIM14, hu9F5VHv9/hu9F5VLv8_DIM17, hu9F5VHv9/hu9F5VLv8_DIM18, hu9F5VHv9/hu9F5VLv8_DIM27, hu9F5VHv9/hu9F5VLv8_DIM28, hu9F5VHv9/hu9F5VLv8_DIM29, hu9F5VHv9/hu9F5VLv8_DIM30, and hu9F5VHv10/hu9F5VLv9_DIM11.

TABLE 25
Affinity of 9F5 Humanized Variants

| | Substitutions | Substitutions | | | |
|---|---|---|---|---|---|
| | in VH relative | in VL relative | | | |
| | to hu9F5VHv9 | to hu9F5VLv8 | | | |
| 9F5 Humanized Variant | (Kabat number | (Kabat number | | | |
| and SEQ ID NOs: | of mutation(s) | of mutation(s) | Kₐ (1/M s) | K_{d} (1/s) | K_{D} (M) |
| hu9F5VHv9/hu9F5VLv8 | | | 1716666.667 | 0.000429333 | 2.69333E−10 |
| SEQ ID NO: 127/ | | | | | |
| SEQ ID NO: 130 | | | | | |
| hu9F5VHv9/hu9F5VLv8_DIM1 | | V3Q, L27cS, | 6620000 | 0.00559 | 8.44E−10 |
| SEQ ID NO: 127/ | | L37Q, M51G, | | | |
| SEQ ID NO: 132 | | L54G, L92I | | | |
| hu9F5VHv9/hu9F5VLv8_DIM2 | | V3Q, L27cS, | 1960000 | 0.000586 | 2.99E−10 |
| SEQ ID NO: 127/ | | L37Q, M51G, | | | |
| SEQ ID NO: 133 | | L54R, L92I | | | |
| hu9F5VHv9/hu9F5VLv8_DIM3 | | V3Q, L27cS, | 1130000 | 0.00068 | 6E−10 |
| SEQ ID NO: 127/ | | L37Q, M51G, | | | |
| SEQ ID NO: 134 | | L54T, L92I | | | |
| hu9F5VHv9/hu9F5VLv8_DIM4 | | V3Q, L27cS, | 542000 | 0.00143 | 2.63E−09 |
| SEQ ID NO: 127/ | | L37Q, M51G, | | | |
| SEQ ID NO: 135 | | L54R, L92G | | | |
| hu9F5VHv9/hu9F5VLv8_DIM5 | | V3Q, L27cG, | 1750000 | 0.000295 | 1.69E−10 |
| SEQ ID NO: 127/ | | L37Q, M51G, | | | |
| SEQ ID NO: 136 | | L54R, L92I | | | |
| hu9F5VHv9/hu9F5VLv8_DIM6 | | V3Q, L27cD, | 1530000 | 0.000272 | 1.78E−10 |
| SEQ ID NO: 127/ | | L37Q, M51G, | | | |
| SEQ ID NO: 137 | | L54R, L92I | | | |
| hu9F5VHv9/hu9F5VLv8_DIM7 | | V3Q, L27cD, | 2360000 | 0.000424 | 1.8E−10 |
| SEQ ID NO: 127/ | | L37Q, M51K, | | | |
| SEQ ID NO: 138 | | L54R, L92I | | | |
| hu9F5VHv9/hu9F5VLv8_DIM8 | | V3Q, L27cG, | 1320000 | 0.000576 | 4.35E−10 |
| SEQ ID NO: 127/ | | L37Q, M51K, | | | |
| SEQ ID NO: 139 | | L54R, L92I | | | |
| hu9F5VHv9/hu9F5VLv8_DIM9 | | V3Q, L27cG, | 2290000 | 0.0116 | 5.07E−09 |
| SEQ ID NO: 127/ | | L37Q, M51K, | | | |
| SEQ ID NO: 140 | | L54G, L92I | | | |
| hu9F5VHv9/hu9F5VLv8_DIM10 | | V3Q, L27cS, | 18900000 | 0.0741 | 3.92E−09 |
| SEQ ID NO: 127/ | | L37Q, M51K, | | | |
| SEQ ID NO: 141 | | L54G, L92I | | | |
| hu9F5VHv9/hu9F5VLv8_DIM11 | | V3Q, L27cG, | 1420000 | 0.00011 | 7.72E−11 |
| SEQ ID NO: 127/ | | L37G, M51G, | | | |
| SEQ ID NO: 142 | | L54R, L92I | | | |
| hu9F5VHv9/hu9F5VLv8_DIM12 | | V3Q, L27cG, | 1940000 | 0.000429 | 2.21E−10 |
| SEQ ID NO: 127/ | | L37G, M51G, | | | |
| SEQ ID NO: 143 | | L54R, L92G | | | |
| hu9F5VHv9/hu9F5VLv8_DIM13 | | V3Q, L27cG, | 1650000 | 0.000439 | 2.66E−10 |
| SEQ ID NO: 127/ | | L37G, M51G, | | | |
| SEQ ID NO: 144 | | L54R | | | |
| hu9F5VHv9/hu9F5VLv8_DIM14 | | V3Q, L27cG, | 1330000 | 0.000188 | 1.42E−10 |
| SEQ ID NO: 127/ | | L37G, M51G, | | | |
| SEQ ID NO: 145 | | L54T, L92I | | | |
| hu9F5VHv9/hu9F5VLv8_DIM15 | | V3Q, L27cG, | 839000 | 0.000732 | 8.73E−10 |
| SEQ ID NO: 127/ | | L37G, M51G, | | | |
| SEQ ID NO: 146 | | L54T, L92G | | | |
| hu9F5VHv9/hu9F5VLv8_DIM16 | | V3Q, L27cG, | 2270000 | 0.000808 | 3.55E−10 |
| SEQ ID NO: 127/ | | L37G, M51G, | | | |
| SEQ ID NO: 147 | | L54T | | | |
| hu9F5VHv9/hu9F5VLv8_DIM17 | | V3Q, L27cS, | 2540000 | 0.000326 | 1.28E−10 |
| SEQ ID NO: 127/ | | L37G, M51G, | | | |
| SEQ ID NO: 148 | | L54T, L92I | | | |
| hu9F5VHv9/hu9F5VLv8_DIM18 | | V3Q, L27cD, | 2100000 | 0.0000939 | 4.47E−11 |
| SEQ ID NO: 127/ | | L37G, M51G, | | | |
| SEQ ID NO: 149 | | L54R, L92I | | | |
| hu9F5VHv9/hu9F5VLv8_DIM19 | | V3Q, L27cS, | 3610000 | 0.00215 | 5.97E−10 |
| SEQ ID NO: 127/ | | L37I, M51I, | | | |
| SEQ ID NO: 150 | | L54R, L92I | | | |
| hu9F5VHv9/hu9F5VLv8_DIM20 | | V3Q, L27cS, | 5640000 | 0.0694 | 1.23E−08 |
| SEQ ID NO: 127/ | | L37Q, M51I, | | | |
| SEQ ID NO: 151 | | L54G, L92I | | | |
| hu9F5VHv9/hu9F5VLv8_DIM21 | | V3Q, L27cS, | 1310000 | 1.12 | 0.000000858 |
| SEQ ID NO: 127/ | | L37Q, M51I, | | | |
| SEQ ID NO: 152 | | L54G | | | |
| hu9F5VHv9/hu9F5VLv8_DIM22 | | V3Q, L27cS, | 2250000 | 0.000878 | 3.9E−10 |
| SEQ ID NO: 127/ | | L37Q, M51E, | | | |
| SEQ ID NO: 153 | | L54R, L92I | | | |
| hu9F5VHv9/hu9F5VLv8_DIM23 | | V3Q, L27cG, | 66900000 | 0.0832 | 1.24E−09 |
| SEQ ID NO: 127/ | | L37Q, M51E, | | | |
| SEQ ID NO: 154 | | L54G, L92I | | | |
| hu9F5VHv9/hu9F5VLv8_DIM24 | | V3Q, L27cG, | 2190000 | 0.000993 | 4.54E−10 |
| SEQ ID NO: 127/ | | L37I, M51E, | | | |
| SEQ ID NO: 155 | | L54R, L92I | | | |
| hu9F5VHv9/hu9F5VLv8_DIM25 | | V3Q, L27cG, | 13000000 | 0.0409 | 3.15E−09 |
| SEQ ID NO: 127/ | | L37I, M51E, | | | |
| SEQ ID NO: 156 | | L54R, L92G | | | |
| hu9F5VHv9/hu9F5VLv8_DIM26 | | V3Q, L27cI, | 3840000 | 0.004 | 1.04E−09 |
| SEQ ID NO: 127/ | | L37I, M51E, | | | |
| SEQ ID NO: 157 | | L54R | | | |
| hu9F5VHv9/hu9F5VLv8_DIM27 | | V3Q, L37Q, | 2410000 | 0.000385 | 1.6E−10 |
| SEQ ID NO: 127 | | M51G, L54R, | | | |
| SEQ ID NO: 158 | | L92I | | | |
| hu9F5VHv9/hu9F5VLv8_DIM28 | | V3Q, L27cS, | 3130000 | 0.000217 | 6.92E−11 |
| SEQ ID NO: 127/ | | M51G, L54R, | | | |
| SEQ ID NO: 159 | | L92I | | | |
| hu9F5VHv9/hu9F5VLv8_DIM29 | | V3Q, L27cS, | 1980000 | 0.000534 | 2.7E−10 |
| SEQ ID NO: 127/ | | L37Q, L54R, | | | |
| SEQ ID NO: 160 | | L92I | | | |
| hu9F5VHv9/hu9F5VLv8_DIM30 | | V3Q, L27cS, | 1020000 | 0.000447 | 4.38E−10 |
| SEQ ID NO: 127/ | | L37Q, M51G, | | | |
| SEQ ID NO: 161 | | L92I | | | |
| hu9F5VHv10/hu9F5VLv9_DIM1 | Q38K, G42E | V3Q, L27cS, | 2690000 | 0.00395 | 1.47E−09 |
| SEQ ID NO: 128/ | | L37Q, M51G, | | | |
| SEQ ID NO: 162 | | L54G, N60D, | | | |
| | | L92I | | | |
| hu9F5VHv10/hu9F5VLv9_DIM2 | Q38K, G42E | V3Q, L27cS, | 2720000 | 0.00184 | 6.79E−10 |
| SEQ ID NO: 128/ | | L37Q, M51G, | | | |
| SEQ ID NO: 163 | | L54R, N60D, | | | |
| | | L92I | | | |
| hu9F5VHv10/hu9F5VLv9_DIM4 | Q38K, G42E | V3Q, L27cS, | 2530000 | 0.00439 | 1.74E−09 |
| SEQ ID NO: 128/ | | L37Q, M51G, | | | |
| SEQ ID NO: 164 | | L54R, N60D, | | | |
| | | L92G | | | |
| hu9F5VHv10/hu9F5VLv9_DIM5 | Q38K, G42E | V3Q, L27cG, | 1700000 | 0.000919 | 5.42E−10 |
| SEQ ID NO: 128/ | | L37Q, M51G, | | | |
| SEQ ID NO: 165 | | L54R, N60D, | | | |
| | | L92I | | | |
| hu9F5VHv10/hu9F5VLv9_DIM8 | Q38K, G42E | V3Q, L27cG, | 2710000 | 0.00232 | 8.59E−10 |
| SEQ ID NO: 128/ | | L37Q, M51K, | | | |
| SEQ ID NO: 166 | | L54R, N60D, | | | |
| | | L92I | | | |
| hu9F5VHv10/hu9F5VLv9_DIM10 | Q38K, G42E | V3Q, L27cS, | 6890000 | 0.026 | 3.77E−09 |
| SEQ ID NO:/ | | L37Q, M51K, | | | |
| SEQ ID NO: 167 | | L54G, N60D, | | | |
| | | L92I | | | |
| hu9F5VHv10/hu9F5VLv9_DIM11 | Q38K, G42E | V3Q, L27cG, | 1760000 | 0.000287 | 1.63E−10 |
| SEQ ID NO: 128/ | | L37G, M51G, | | | |
| SEQ ID NO: 168 | | L54R, N60D, | | | |
| | | L92I | | | |
| hu9F5VHv10/hu9F5VLv9_DIM13 | Q38K, G42E | V3Q, L27cG, | 2280000 | 0.00116 | 5.08E−10 |
| SEQ ID NO: 128/ | | L37G, M51G, | | | |
| SEQ ID NO: 169 | | L54R, N60D, | | | |
| hu9F5VHv10/hu9F5VLv9_DIM19 | Q38K, G42E | V3Q, L27cS, | 3490000 | 0.00688 | 1.97E−09 |
| SEQ ID NO: 128/ | | L37I, M51I, | | | |
| SEQ ID NO: 170 | | L54R, N60D, | | | |
| | | L92I | | | |
| hu9F5VHv10/hu9F5VLv9_DIM20 | Q38K, G42E | V3Q, L27cS, | 3470000 | 0.0334 | 9.62E−09 |
| SEQ ID NO: 128/ | | L37Q, M51I, | | | |
| SEQ ID NO: 171 | | L54G, N60D, | | | |
| | | L92I | | | |

In addition, as a secondary characteristic, thermostability and titer were analyzed for all deimmunized variants. Thermostability and titer levels were compared for antibodies that were comparable to Hu9F5VHv9/Hu9F5VLv8 based on affinity measurements, and antibodies in Table 26 are listed in order based on variance from T. of Hu9F5VHv9/Hu9F5VLv8.

Thermostability values were determined using differential scanning calorimetry (DSC). All DSC scans were performed using a VP-Capillary DSC system (Malvern). All samples were prepared to 0.5 mg/mL in1× PBS, and referenced to 1× PBS. Approximately 0.5 mL of protein solution and buffer were introduced into the sample and reference cell. calorimetric scan rates were carried out at scan rates of 60° C./hour, from 25° C. to 110° C. under constant pressure. Analysis was performed using origin software. Reported values are the temperature at which the maximal heat capacity of the Fab peak is recorded.

Titer was determined as follows. After expression in 293 suspension cells, antibodies were purified using Protein A chromatography utilizing standard methods. Following purification, the antibodies were exchanged into 1× PBS and protein concentrations were determined by absorbance at 280nm. Titers were calculated by dividing the final yield of purified protein by the starting volume of the expression culture, and reported in milligrams per liter.

TABLE 26
Thermostability and Titer of 9F5 Humanized Variants

| | Substitutions | Substitutions | Tₘ (° C.)- | |
|---|---|---|---|---|
| | in VH relative | in VL relative | variance of Fab | |
| | to hu9F5VHv9 | to hu9F5VLv8 | peak from | |
| | (Kabat number | (Kabat number | hu9F5VHv9/ | titer |
| 9F5 Humanized Variant | of mutation(s) | of mutation(s) | hu9F5VLv8 | (mg/L) |
| hu9F5VHv9/hu9F5VLv8 | | | 0 | 491.6 |
| SEQ ID NO: 127/ | | | | |
| SEQ ID NO: 130 | | | | |
| hu9F5VHv9/hu9F5VLv8_DIM18 | | V3Q, L27cD, | −1.55 | 579.60 |
| SEQ ID NO: 127/ | | L37G, M51G, | | |
| SEQ ID NO: 149 | | L54R, L92I | | |
| hu9F5VHv9/hu9F5VLv8_DIM11 | | V3Q, L27cG, | −3.06 | 446.40 |
| SEQ ID NO: 127/ | | L37G, M51G, | | |
| SEQ ID NO: 142 | | L54R, L92I | | |
| hu9F5VHv9/hu9F5VLv8_DIM28 | | V3Q, L27cS, | −0.27 | 348.70 |
| SEQ ID NO: 127/ | | M51G, L54R, | | |
| SEQ ID NO: 159 | | L92I | | |
| hu9F5VHv9/hu9F5VLv8_DIM17 | | V3Q, L27cS, | −3.83 | 423.80 |
| SEQ ID NO: 127/ | | L37G, M51G, | | |
| SEQ ID NO: 148 | | L54T, L92I | | |
| hu9F5VHv9/hu9F5VLv8_DIM6 | | V3Q, L27cD, | −0.77 | 569.80 |
| SEQ ID NO: 127/ | | L37Q, M51G, | | |
| SEQ ID NO: 137 | | L54R, L92I | | |
| hu9F5VHv9/hu9F5VLv8_DIM14 | | V3Q, L27cG, | −5.65 | 282.40 |
| SEQ ID NO: 127/ | | L37G, M51G, | | |
| SEQ ID NO: 145 | | L54T, L92I | | |
| hu9F5VHv9/hu9F5VLv8_DIM5 | | V3Q, L27cG, | −1.83 | 287.30 |
| SEQ ID NO: 127/ | | L37Q, M51G, | | |
| SEQ ID NO: 136 | | L54R, L92I | | |
| hu9F5VHv9/hu9F5VLv8_DIM7 | | V3Q, L27cD, | −2.05 | 636.60 |
| SEQ ID NO: 127/ | | L37Q, M51K, | | |
| SEQ ID NO: 138 | | L54R, L92I | | |
| hu9F5VHv9/hu9F5VLv8_DIM27 | | V3Q, L37Q, | −0.16 | 351.20 |
| SEQ ID NO: 127/ | | M51G, L54R, | | |
| SEQ ID NO: 158 | | L92I | | |
| hu9F5VHv9/hu9F5VLv8_DIM12 | | V3Q, L27cG, | −6.38 | 369.40 |
| SEQ ID NO: 127/ | | L37G, M51G, | | |
| SEQ ID NO: 143 | | L54R, L92G | | |
| hu9F5VHv9/hu9F5VLv8_DIM13 | | V3Q, L27cG, | −1.7 | 595.80 |
| SEQ ID NO: 127/ | | L37G, M51G, | | |
| SEQ ID NO: 144 | | L54R | | |
| hu9F5VHv9/hu9F5VLv8_DIM2 | | V3Q, L27cS, | −0.3 | 474.30 |
| SEQ ID NO: 127/ | | L37Q, M51G, | | |
| SEQ ID NO: 133 | | L54R, L92I | | |
| hu9F5VHv9/hu9F5VLv8_DIM29 | | V3Q, L27cS, | −1.83 | 327.50 |
| SEQ ID NO: 127/ | | L37Q, L54R, | | |
| SEQ ID NO: 160 | | L92I | | |
| hu9F5VHv9/hu9F5VLv8_DIM30 | | V3Q, L27cS, | −0.92 | 372.50 |
| SEQ ID NO: 127/ | | L37Q, M51G, | | |
| SEQ ID NO: 161 | | L92I | | |
| hu9F5VHv9/hu9F5VLv8_DIM8 | | V3Q, L27cG, | −4.89 | 451.50 |
| SEQ ID NO: 127/ | | L37Q, M51K, | | |
| SEQ ID NO: 139 | | L54R, L92I | | |
| hu9F5VHv10/hu9F5VLv9_DIM11 | Q38K, G42E | V3Q, L27cG, | −11.94 | 244.60 |
| SEQ ID NO: 128/ | | L37G, M51G, | | |
| SEQ ID NO: 168 | | L54R, N60D, | | |
| | | L92I | | |

### Example 8 Neutralization Activity of Humanized 9F5 Variants

Neutralization activity of humanized 9F5 variants is analyzed in a cell based model of tau internalization. An internalization assay employing fluorescence activated cell sorting (FACS) is performed to evaluate the ability of antibodies to block neuronal internalization of tau. Antibodies that block internalization will likely block transmission of tau. pHrodo-labeled 4R0N human tau P301L soluble oligomer (1.5 μg/ml final concentration) is preincubated with anti-tau antibodies (dose titration: 80 μg/ml starting concentration followed by 4-fold serial dilutions) for 30 min at room temperature in cell culture media. Tau/antibody mixture is then added to B103 neuroblastoma cell lines at 500,000 cells/ml final concentration and incubated for 3-4 hrs at 37° C. in a tissue culture incubator (5% CO₂). Cells are then washed 3× with culture media, followed by 10 minutes culture media incubation, and washed 2× with FACS buffer (1% FBS in PBS). Cells are resuspended in 100 μl FACS buffer and Texas Red mean fluorescence intensity measured by FACS LSR II. Texas red fluorescence from pHrodo is activated by low pH associated with endolysosomal compartments upon internalization. Because FACS detects cells and pHrodo only fluoresces upon internalization, only tau internalized by the cells will be detected. The lower the mean fluorescence intensity, the lower the amount of internalized tau, which suggests a higher blocking activity of the antibody tested.

### Example 9. Design of Humanized 10C12 Antibodies

The starting point or donor antibody for humanization was the mouse antibody 10C12. The heavy chain variable amino acid sequence of mature m10C12 is provided as SEQ ID NO:7. The light chain variable amino acid sequence of mature m10C12 is provided as SEQ ID NO:11. The heavy chain Kabat/Chothia Composite CDR1, CDR2, and CDR3 amino acid sequences are provided as SEQ ID NOs:8-10, respectively. The light chain Kabat CDR1, CDR2, and CDR3 amino acid sequences are provided as SEQ ID NOs:12-14 respectively. Kabat numbering is used throughout.

The variable kappa (Vk) of 10C12 belongs to mouse Vk subgroup 2 which corresponds to human Vk subgroup 2 and the variable heavy (Vh) to mouse Vh subgroup 2c which corresponds to human Vh subgroup 1 [Kabat E. A., et al., (1991), Sequences of Proteins of Immunological Interest, Fifth Edition. NIH Publication No. 91-3242]. 16 residue Chothia CDR-L1 is similar to Chothia canonical class 4, 7 residue Chothia CDR-L2 is of Chothia canonical class 1, 9 residue Chothia CDR-L3 is similar to Chothia canonical class 1 [Martin ACR. (2010) Protein sequence and structure analysis of antibody variable domains. In: Kontermann R and Dübel S (eds). Antibody Engineering. Heidelberg, Germany: Springer International Publishing AG. [Martin, 2010]. 10 residue Chothia CDR-H1 is similar to Chothia canonical class 1, 17 residue Chothia CDR-H2 is similar to Chothia canonical class 2 [Martin, 2010]. 3 residue CDR-H3 has no canonical classes. A search was made over the protein sequences in the PDB database [Deshpande N, et al., (2005) Nucleic Acids Res. 33: D233-7] to find structures, which would provide a rough structural model of 10C12. The crystal structure of an antibody fab pdb code 5OBF [Vicentini, et al., 2017, unpublished] was used for both Vh and Vk structure since it had good resolution (1.92A°) and overall sequence similarity to 10C12 Vh and Vk, retaining the same canonical structures for the loops.

The frameworks of 10C12 VH share a high degree of sequence similarity with the corresponding regions of immunoglobulin heavy chain variable region [Homo sapiens] CAC20421 cloned by Arnold-Schild et al [Cancer Res. 60 (15), 4175-4178 (2000)]. The variable domains of 10C12 and CAC20421 VH also share identical lengths for the CDR-H1, H2 loops. Similarly, the frameworks of 10C12 VL share a high degree of sequence similarity with the corresponding regions of human antibody CAB51297, cloned by Capello et al [Identification of three subgroups of B-cell chronic lymphocytic leukemia based upon mutations of BCL-6 and IGV genes. Unpublished (direct submission to GenBank)]. The variable light domain of 10C12 and CAB51297 VL also share identical lengths for the CDR-L1, L2 and L3 loops. Accordingly, the framework regions of CAC20421 VH and CAB51297 VL were chosen as the acceptor sequences for the CDRs of 10C12. A model of the 10C12 CDRs grafted onto the respective human frame-works for VH and VL was built and used as a guidance for further backmutations.

Heavy and light chain variant sequences resulting from antibody humanization process were further aligned to human germ line sequences using IMGT Domain GapAlign tool to assess the humanness of the heavy and light chain as outlined by WHO INN committee guidelines. (WHO-INN: International nonproprietary names (INN) for biological and biotechnological substances (a review) (Internet) 2014. Available from: http://www.who.int/medicines/services/inn/BioRev2014.pdf) Residues were changed to align with corresponding human germ line sequence, where possible, to enhance humanness and to reduce potential immunogenicity. For humanized VLv1 and VLv2, variants, mutations were introduced to render the sequences more similar to human germ line gene IGKV2-28*01 & IGKJ2*01 (SEQ ID NO:37). For humanized VHv1 and VHv2, variants, mutations were introduced to render the sequences more similar to human germ line gene IGHV1-69-2*01 (SEQ ID NO:33)

Additional versions of hu10C12-VH and hu10C12-VL were designed to enable assessment of various framework residues for their contributions to antigen binding, thermostability, and immunogenicity, and for optimization of deamination, oxidation, N-glycosylation, proteolysis and aggregation. The positions considered for mutation include those that:
define the canonical CDR conformations (summarized in Martin, A. C. R. (2010) Protein sequence and structure analysis of antibody variable domains. In: Kontermann R and Dübel S (eds). Antibody Engineering. Heidelberg, Germany: Springer International Publishing AG.),
are within the Vernier zone (Foote J and Winter G. (1992) Antibody framework residues affecting the conformation of the hypervariable loops. J Mol Biol. 224(2):487-99.),
localize to the VH/VL domain interface (summarized in Léger O J P and Saldanha J. (2000) Preparation of recombinant antibodies from immune rodent spleens and the design of their humanisation by CDR grafting. In: Shepherd P and Dean C (eds). Monoclonal Antibodies: a Practical Approach. Oxford, UK: Oxford University Press.),
are susceptible to post-translational modifications, such as glycosylation or pyroglutamination,
are occupied by residues that are predicted to clash with CDRs, according to the model of 10C12 CDRs grafted onto VH and VL frameworks, or
are occupied by residues that are rare among sequenced human antibodies, where either the parental mouse 10C12 residue or some other residue is much more prevalent within human antibody repertoire.

Alignments of the murine 10C12 and various humanized antibodies are shown for the light chain variable regions (Table 28 and FIG. 8), and heavy chain variable regions (Table 27 and FIG. 7).

2 humanized heavy chain variable region variants and 2 humanized light chain variable region variants were constructed containing different permutations of substitutions: hu10C12VHv1 or hu10C12VHv2, (SEQ ID NOs:214-215, respectively); and hu10C12VLv1 or hu10C12VLv2, (SEQ ID NOs:216-217, respectively) (Tables 27 and 28). The exemplary humanized Vk and Vh designs, with backmutations and other mutations based on selected human frameworks, are shown in Tables 27 and 28, respectively. The bolded areas in Tables27 and 28 indicate the CDRs as defined by Kabat/Chothia Composite. A “-” in the columns in Tables 27 and 28 indicates no residue at the indicated position. SEQ ID NOs:214-215, and SEQ ID NOs:216-217 contain backmutations and other mutations as shown in Table 29. The amino acids at positions in hu10C12VHv1 and hu10C12VHv2 are listed in Table 30. The amino acids at positions in hu10C12VLv1 and hu10C12VLv2 are listed in Table 31.

The percentage humanness for humanized VH chains hu10C12VHv1 and hu10C12VHv2 (SEQ ID NOs:214-215, respectively) with respect to the most similar human germline gene IGHV1-69-2*01 (SEQ ID NO:33), and for humanized VL chains hu10C12VLv1 and hu10C12VLv2 (SEQ ID NOs:216-217, respectively) with respect to the most similar human germline gene IGKV2-28*01 & IGKJ2*01 (SEQ ID NO:37), is shown in Table 32.

TABLE 27

| | | | | IMGT# | CAC20421- | | |
|---|---|---|---|---|---|---|---|
| | | | Mouse 10C12 VH | IGV1-69-2*01 | VH_huFrwk | Hu10C12VHv1 | Hu10C12VHv2 |
| Kabat | Linear | FD or | (SEQ ID | (SEQ ID | (SEQ ID | (SEQ ID | SEQ ID |
| residue # | residue # | CDR | NO: 7) | NO: 33) | NO: 218) | NO: 214) | NO: 215) |
| 1 | 1 | Fr1 | E | E | Q | Q | E |
| 2 | 2 | Fr1 | V | V | V | V | V |
| 3 | 3 | Fr1 | Q | Q | Q | Q | Q |
| 4 | 4 | Fr1 | L | L | L | L | L |
| 5 | 5 | Fr1 | Q | V | V | V | V |
| 6 | 6 | Fr1 | Q | Q | Q | Q | Q |
| 7 | 7 | Fr1 | S | S | S | S | S |
| 8 | 8 | Fr1 | G | G | G | G | G |
| 9 | 9 | Fr1 | A | A | A | A | A |
| 10 | 10 | Fr1 | E | E | E | E | E |
| 11 | 11 | Fr1 | L | V | V | V | V |
| 12 | 12 | Fr1 | V | K | K | K | K |
| 13 | 13 | Fr1 | R | K | K | K | K |
| 14 | 14 | Fr1 | P | P | P | P | P |
| 15 | 15 | Fr1 | G | G | G | G | G |
| 16 | 16 | Fr1 | A | A | A | A | A |
| 17 | 17 | Fr1 | S | T | T | T | T |
| 18 | 18 | Fr1 | V | V | V | V | V |
| 19 | 19 | Fr1 | K | K | K | K | K |
| 20 | 20 | Fr1 | L | I | I | I | I |
| 21 | 21 | Fr1 | S | S | S | S | S |
| 22 | 22 | Fr1 | C | C | C | C | C |
| 23 | 23 | Fr1 | T | K | K | K | K |
| 24 | 24 | Fr1 | A | V | V | A | A |
| 25 | 25 | Fr1 | S | S | S | S | S |
| 26 | 26 | CDR-H1 | G | G | G | G | G |
| 27 | 27 | CDR-H1 | F | Y | Y | F | F |
| 28 | 28 | CDR-H1 | N | T | T | N | N |
| 29 | 29 | CDR-H1 | I | F | F | I | I |
| 30 | 30 | CDR-H1 | K | T | T | K | K |
| 31 | 31 | CDR-H1 | D | D | D | D | D |
| 32 | 32 | CDR-H1 | D | Y | Y | D | D |
| 33 | 33 | CDR-H1 | Y | Y | Y | Y | Y |
| 34 | 34 | CDR-H1 | M | M | M | M | M |
| 35 | 35 | CDR-H1 | N | H | H | N | N |
| 35A | | CDR-H1 | — | — | — | — | — |
| 35B | | CDR-H1 | — | — | — | — | — |
| 36 | 36 | Fr2 | W | W | W | W | W |
| 37 | 37 | Fr2 | V | V | V | V | V |
| 38 | 38 | Fr2 | K | Q | Q | Q | Q |
| 39 | 39 | Fr2 | Q | Q | Q | Q | Q |
| 40 | 40 | Fr2 | R | A | A | A | A |
| 41 | 41 | Fr2 | P | P | P | P | P |
| 42 | 42 | Fr2 | E | G | G | G | G |
| 43 | 43 | Fr2 | R | K | K | K | K |
| 44 | 44 | Fr2 | G | G | G | G | G |
| 45 | 45 | Fr2 | L | L | L | L | L |
| 46 | 46 | Fr2 | E | E | E | E | E |
| 47 | 47 | Fr2 | W | W | W | W | W |
| 48 | 48 | Fr2 | I | M | M | I | I |
| 49 | 49 | Fr2 | G | G | G | G | G |
| 50 | 50 | CDR-H2 | W | L | L | W | W |
| 51 | 51 | CDR-H2 | I | V | V | I | I |
| 52 | 52 | CDR-H2 | D | D | D | D | D |
| 52A | 53 | CDR-H2 | P | P | P | P | P |
| 52B | | CDR-H2 | — | — | — | — | — |
| 52C | | CDR-H2 | — | — | — | — | — |
| 53 | 54 | CDR-H2 | E | E | E | E | E |
| 54 | 55 | CDR-H2 | N | D | D | N | N |
| 55 | 56 | CDR-H2 | G | G | G | G | G |
| 56 | 57 | CDR-H2 | D | E | E | D | D |
| 57 | 58 | CDR-H2 | T | T | T | T | T |
| 58 | 59 | CDR-H2 | E | I | I | E | E |
| 59 | 60 | CDR-H2 | Y | Y | Y | Y | Y |
| 60 | 61 | CDR-H2 | A | A | A | A | A |
| 61 | 62 | CDR-H2 | S | E | E | S | S |
| 62 | 63 | CDR-H2 | K | K | K | K | K |
| 63 | 64 | CDR-H2 | F | F | F | F | F |
| 64 | 65 | CDR-H2 | Q | Q | Q | Q | Q |
| 65 | 66 | CDR-H2 | G | G | G | G | G |
| 66 | 67 | Fr3 | K | R | R | R | R |
| 67 | 68 | Fr3 | A | V | V | A | A |
| 68 | 69 | Fr3 | T | T | T | T | T |
| 69 | 70 | Fr3 | M | I | I | M | M |
| 70 | 71 | Fr3 | T | T | T | T | T |
| 71 | 72 | Fr3 | A | A | A | A | A |
| 72 | 73 | Fr3 | D | D | D | D | D |
| 73 | 74 | Fr3 | T | T | T | T | T |
| 74 | 75 | Fr3 | S | S | S | S | S |
| 75 | 76 | Fr3 | S | T | T | T | T |
| 76 | 77 | Fr3 | N | D | D | D | D |
| 77 | 78 | Fr3 | T | T | T | T | T |
| 78 | 79 | Fr3 | A | A | A | A | A |
| 79 | 80 | Fr3 | Y | Y | Y | Y | Y |
| 80 | 81 | Fr3 | L | M | M | M | M |
| 81 | 82 | Fr3 | Q | E | E | E | E |
| 82 | 83 | Fr3 | F | L | L | L | L |
| 82A | 84 | Fr3 | S | S | S | S | S |
| 82B | 85 | Fr3 | S | S | S | S | S |
| 82C | 86 | Fr3 | L | L | L | L | L |
| 83 | 87 | Fr3 | T | R | R | R | R |
| 84 | 88 | Fr3 | S | S | S | S | S |
| 85 | 89 | Fr3 | E | E | E | E | E |
| 86 | 90 | Fr3 | D | D | D | D | D |
| 87 | 91 | Fr3 | T | T | T | T | T |
| 88 | 92 | Fr3 | A | A | A | A | A |
| 89 | 93 | Fr3 | V | V | V | V | V |
| 90 | 94 | Fr3 | Y | Y | Y | Y | Y |
| 91 | 95 | Fr3 | Y | Y | Y | Y | Y |
| 92 | 96 | Fr3 | C | C | C | C | C |
| 93 | 97 | Fr3 | T | A | A | T | T |
| 94 | 98 | Fr3 | T | T | R | T | T |
| 95 | 99 | CDR-H3 | S | — | I | S | S |
| 96 | | CDR-H3 | — | — | P | — | — |
| 97 | | CDR-H3 | — | — | L | — | — |
| 98 | | CDR-H3 | — | — | F | — | — |
| 99 | | CDR-H3 | — | — | G | — | — |
| 100 | | CDR-H3 | — | — | R | — | — |
| 100A | | CDR-H3 | — | — | — | — | — |
| 100B | | CDR-H3 | — | — | — | — | — |
| 100C | | CDR-H3 | — | — | — | — | — |
| 100D | | CDR-H3 | — | — | — | — | — |
| 100E | | CDR-H3 | — | — | — | — | — |
| 100F | | CDR-H3 | — | — | — | — | — |
| 100G | | CDR-H3 | — | — | — | — | — |
| 100H | | CDR-H3 | — | — | — | — | — |
| 100I | | CDR-H3 | — | — | — | — | — |
| 100J | | CDR-H3 | — | — | — | — | — |
| 100K | | CDR-H3 | — | — | — | — | — |
| 101 | 100 | CDR-H3 | N | Q | D | N | N |
| 102 | 101 | CDR-H3 | G | H | H | G | G |
| 103 | 102 | Fr4 | W | W | W | W | W |
| 104 | 103 | Fr4 | G | G | G | G | G |
| 105 | 104 | Fr4 | Q | Q | Q | Q | Q |
| 106 | 105 | Fr4 | G | G | G | G | G |
| 107 | 106 | Fr4 | T | T | T | T | T |
| 108 | 107 | Fr4 | L | L | L | L | L |
| 109 | 108 | Fr4 | V | V | V | V | V |
| 110 | 109 | Fr4 | T | T | T | T | T |
| 111 | 110 | Fr4 | V | V | V | V | V |
| 112 | 111 | Fr4 | S | S | S | S | S |
| 113 | 112 | Fr4 | T | S | S | S | S |

TABLE 28

| | | | | IGKV2-28*01 & | CAB51297- | | |
|---|---|---|---|---|---|---|---|
| | | | Mouse 10C12 VL | IGKJ2*01 | VL_huFrwk | Hu10C12VLv1 | Hu10C12VLv2 |
| Kabat | Linear | FR or | (SEQ ID | (SEQ ID | (SEQ ID | (SEQ ID | (SEQ ID |
| residue # | residue # | CDR | NO: 11) | NO:37) | NO: 35) | NO: 216) | NO: 217) |
| 1 | 1 | Fr1 | D | D | D | D | D |
| 2 | 2 | Fr1 | I | I | I | I | I |
| 3 | 3 | Fr1 | V | V | V | V | V |
| 4 | 4 | Fr1 | M | M | M | M | M |
| 5 | 5 | Fr1 | T | T | T | T | T |
| 6 | 6 | Fr1 | Q | Q | Q | Q | Q |
| 7 | 7 | Fr1 | A | S | S | S | S |
| 8 | 8 | Fr1 | A | P | P | P | P |
| 9 | 9 | Fr1 | F | L | L | L | L |
| 10 | 10 | Fr1 | S | S | S | S | S |
| 11 | 11 | Fr1 | N | L | L | L | L |
| 12 | 12 | Fr1 | P | P | P | P | P |
| 13 | 13 | Fr1 | V | V | V | V | V |
| 14 | 14 | Fr1 | T | T | T | T | T |
| 15 | 15 | Fr1 | L | P | P | P | P |
| 16 | 16 | Fr1 | G | G | G | G | G |
| 17 | 17 | Fr1 | T | E | E | E | E |
| 18 | 18 | Fr1 | S | P | P | P | P |
| 19 | 19 | Fr1 | A | A | A | A | A |
| 20 | 20 | Fr1 | S | S | S | S | S |
| 21 | 21 | Fr1 | I | I | I | I | I |
| 22 | 22 | Fr1 | S | S | S | S | S |
| 23 | 23 | Fr1 | C | C | C | C | C |
| 24 | 24 | CDR-L1 | R | R | R | R | R |
| 25 | 25 | CDR-L1 | S | S | S | S | S |
| 26 | 26 | CDR-L1 | S | S | S | S | S |
| 27 | 27 | CDR-L1 | K | Q | Q | K | K |
| 27A | 28 | CDR-L1 | S | S | S | S | S |
| 27B | 29 | CDR-L1 | L | L | L | L | L |
| 27C | 30 | CDR-L1 | L | L | L | L | L |
| 27D | 31 | CDR-L1 | H | H | H | H | H |
| 27E | 32 | CDR-L1 | S | S | S | S | S |
| 27F | | CDR-L1 | — | — | — | — | — |
| 28 | 33 | CDR-L1 | N | N | N | N | N |
| 29 | 34 | CDR-L1 | G | G | G | G | G |
| 30 | 35 | CDR-L1 | I | Y | Y | I | I |
| 31 | 36 | CDR-L1 | T | N | N | T | T |
| 32 | 37 | CDR-L1 | Y | Y | Y | Y | Y |
| 33 | 38 | CDR-L1 | L | L | L | L | L |
| 34 | 39 | CDR-L1 | Y | D | D | Y | Y |
| 35 | 40 | Fr2 | W | W | W | W | W |
| 36 | 41 | Fr2 | Y | Y | Y | Y | Y |
| 37 | 42 | Fr2 | L | L | L | L | L |
| 38 | 43 | Fr2 | Q | Q | Q | Q | Q |
| 39 | 44 | Fr2 | K | K | K | K | K |
| 40 | 45 | Fr2 | P | P | P | P | P |
| 41 | 46 | Fr2 | G | G | G | G | G |
| 42 | 47 | Fr2 | Q | Q | Q | Q | Q |
| 43 | 48 | Fr2 | S | S | S | S | S |
| 44 | 49 | Fr2 | P | P | P | P | P |
| 45 | 50 | Fr2 | Q | Q | Q | Q | Q |
| 46 | 51 | Fr2 | L | L | L | L | L |
| 47 | 52 | Fr2 | L | L | L | L | L |
| 48 | 53 | Fr2 | I | I | I | I | I |
| 49 | 54 | Fr2 | Y | Y | Y | Y | Y |
| 50 | 55 | CDR-L2 | Q | L | L | Q | Q |
| 51 | 56 | CDR-L2 | M | G | G | M | M |
| 52 | 57 | CDR-L2 | S | S | S | S | S |
| 53 | 58 | CDR-L2 | N | N | N | N | N |
| 54 | 59 | CDR-L2 | L | R | R | L | L |
| 55 | 60 | CDR-L2 | A | A | A | A | A |
| 56 | 61 | CDR-L2 | S | S | S | S | S |
| 57 | 62 | Fr3 | G | G | G | G | G |
| 58 | 63 | Fr3 | V | V | V | V | V |
| 59 | 64 | Fr3 | P | P | P | P | P |
| 60 | 65 | Fr3 | D | D | D | D | D |
| 61 | 66 | Fr3 | R | R | R | R | R |
| 62 | 67 | Fr3 | F | F | F | F | F |
| 63 | 68 | Fr3 | S | S | S | S | S |
| 64 | 69 | Fr3 | S | G | G | S | S |
| 65 | 70 | Fr3 | S | S | S | S | S |
| 66 | 71 | Fr3 | G | G | G | G | G |
| 67 | 72 | Fr3 | S | S | S | S | S |
| 68 | 73 | Fr3 | G | G | G | G | G |
| 69 | 74 | Fr3 | T | T | T | T | T |
| 70 | 75 | Fr3 | D | D | D | D | D |
| 71 | 76 | Fr3 | F | F | F | F | F |
| 72 | 77 | Fr3 | T | T | T | T | T |
| 73 | 78 | Fr3 | L | L | L | L | L |
| 74 | 79 | Fr3 | R | K | K | K | K |
| 75 | 80 | Fr3 | I | I | I | I | I |
| 76 | 81 | Fr3 | S | S | S | S | S |
| 77 | 82 | Fr3 | R | R | R | R | R |
| 78 | 83 | Fr3 | V | V | V | V | V |
| 79 | 84 | Fr3 | E | E | E | E | E |
| 80 | 85 | Fr3 | A | A | A | A | A |
| 81 | 86 | Fr3 | E | E | E | E | E |
| 82 | 87 | Fr3 | D | D | D | D | D |
| 83 | 88 | Fr3 | V | V | V | V | V |
| 84 | 89 | Fr3 | G | G | G | G | G |
| 85 | 90 | Fr3 | V | V | V | V | V |
| 86 | 91 | Fr3 | Y | Y | Y | Y | Y |
| 87 | 92 | Fr3 | Y | Y | Y | Y | Y |
| 88 | 93 | Fr3 | C | C | C | C | C |
| 89 | 94 | CDR-L3 | A | M | M | A | A |
| 90 | 95 | CDR-L3 | Q | Q | Q | Q | Q |
| 91 | 96 | CDR-L3 | N | A | A | N | N |
| 92 | 97 | CDR-L3 | L | L | L | L | L |
| 93 | 98 | CDR-L3 | E | Q | Q | E | E |
| 94 | 99 | CDR-L3 | L | T | T | L | L |
| 95 | 100 | CDR-L3 | P | P | P | P | P |
| 95A | | CDR-L3 | — | — | — | — | — |
| 95B | | CDR-L3 | — | — | — | — | — |
| 95C | | CDR-L3 | — | — | — | — | — |
| 95D | | CDR-L3 | — | — | — | — | — |
| 95E | | CDR-L3 | — | — | — | — | — |
| 95F | | CDR-L3 | — | — | — | — | — |
| 96 | 101 | CDR-L3 | L | Y | L | L | L |
| 97 | 102 | CDR-L3 | T | T | T | T | T |
| 98 | 103 | Fr4 | F | F | F | F | F |
| 99 | 104 | Fr4 | G | G | G | G | G |
| 100 | 105 | Fr4 | A | Q | G | G | G |
| 101 | 106 | Fr4 | G | G | G | G | G |
| 102 | 107 | Fr4 | T | T | T | T | T |
| 103 | 108 | Fr4 | K | K | K | K | K |
| 104 | 109 | Fr4 | L | L | V | V | L |
| 105 | 110 | Fr4 | E | E | E | E | E |
| 106 | 111 | Fr4 | L | I | I | I | I |
| 106A | | Fr4 | — | — | — | — | — |
| 107 | 112 | Fr4 | K | K | K | K | K |

TABLE 29
V_{H}, V_{L} Backmutations and Other Mutations for Humanized 10C12

| | | Changes from Acceptor Framework |
|---|---|---|
| | | (or CDR) Residues (based on |
| V_{H} or V_{L} Variant | V_{H} or V_{L} Exon Acceptor Sequence | Kabat/Chothia Composite CDRs) |
| Hu10C12VHv1 | GenBank Acc. # CAC20421-VH_huFrwk | H24, H48, H67, H69, H93, H94 |
| (SEQ ID NO: 214) | (SEQ ID NO: 218) | |
| | IMGT# IGHV1-69-2*01 | |
| | (SEQ ID NO: 33) | |
| Hu10C12VHv2 | GenBank Acc. # CAC20421-VH_huFrwk | H1, H24, H48, H67, H69, H93, H94 |
| (SEQ ID NO: 215) | (SEQ ID NO: 218) | |
| | IMGT# IGHV1-69-2*01 | |
| | (SEQ ID NO: 33) | |
| Hu10C12VLv1 | GenBank Acc. # CAB51297-VL_huFrwk | L64 |
| (SEQ ID NO: 216) | (SEQ ID NO: 35) | |
| Hu10C12VLv2 | GenBank Acc. # CAB51297-VL_huFrwk | L64, L104 |
| (SEQ ID NO: 217) | (SEQ ID NO: 35) | |
| | IGKV2-28*01 & IGKJ2*01 | |
| | (SEQ ID NO: 37) | |

TABLE 30
Kabat Numbering of Framework (or CDR) Residues (based on Kabat/Chothia
Composite CDRs) for Backmutations and Other Mutations in Heavy Chains
of Humanized 10C12 Antibodies

| | CAC20421- | IMGT# | | | |
|---|---|---|---|---|---|
| | VH_huFrwk | IGHV1-69-2*01 | Mouse 10C12 VH | Hu10C12VHv1 | Hu10C12VHv2 |
| | (SEQ ID | (SEQ ID | (SEQ ID | (SEQ ID | (SEQ ID |
| Residue | NO: 218) | NO: 33) | NO: 7) | NO: 214) | NO: 215) |
| H1 | Q | E | E | Q | E |
| H24 | V | V | A | A | A |
| H48 | M | M | I | I | I |
| H67 | V | V | A | A | A |
| H69 | I | I | M | M | M |
| H93 | A | A | T | T | T |
| H94 | R | T | T | T | T |

TABLE 31
Kabat Numbering of Framework Residues (based on Kabat/Chothia
Composite CDRs) for Backmutations and Other Mutations in Light
Chains of Humanized 10C12 Antibodies

| | CAB51297- | IGKV2-28*01 & | | | |
|---|---|---|---|---|---|
| | VL_huFrwk | IGKJ2*01 | Mouse 10C12 VL | Hu10C12VLv1 | Hu10C12VLv2 |
| | (SEQ ID | (SEQ ID | (SEQ ID | (SEQ ID | (SEQ ID |
| Residue | NO: 35) | NO: 37) | NO: 11) | NO: 216) | NO: 217) |
| L64 | G | G | S | S | S |
| L104 | V | L | L | V | L |

TABLE 32
Percentage Humanness of Heavy and Light
Chains of Humanized 10C12 Antibodies

| V_{H} or V_{L} Variant | % Humanness |
|---|---|
| Hu10C12VHv1 (SEQ ID NO: 214) | 81.6% |
| Hu10C12VHv2 (SEQ ID NO: 215) | 82.7% |
| Hu10C12VLv1 (SEQ ID NO: 216) | 88.0% |
| Hu10C12VLv2(SEQ ID NO: 217) | 88.0% |

Positions at which canonical, vernier, or interface residues differ between mouse and human acceptor sequences are candidates for substitution. Examples of canonical/CDR interacting residues include Kabat residues H24 and H94 in Table 27. Examples of vernier residues include Kabat residues H48, H67, H69, H93, and H94 in Table 27 and L64 in Table 28. Examples of interface/packing (VH+VL) residues include Kabat residue H93 in Table 27.

The rationales for selection of the positions indicated in Table 27 in the heavy chain variable region as candidates for substitution are as follows.

Heavy Chain Variable Regions

hu10C12VHv1
consists of the CDR-H1, H2, and H3 loops of 10C12-VH grafted onto the framework of CAC20421-VH. Additionally, hu10C12VHv1 also reverts all framework substitutions at positions that are key for defining the Chothia canonical classes, are part of the Vernier zone, or localize to the VH/VL domain interface contribute to structural stability.

V24A: is a backmutation of a Vernier zone residue.

M48I: is a backmutation of a Vernier zone residue.

V67A: is a backmutation of a Vernier zone residue and is back mutated to retain CDR conformation.

I69M: is a backmutation of a Vernier zone residue and backmutation is made to keep the CDR packing intact.

A93T: Position H93 is a VH/VL interface residue, and is backmutated to Thr for the integrity of the antibody interface.

R94T: is a backmutation of a Chothia canonical structural residue. R94T is also a germ-line-aligning mutation. Thr is in human germ line gene IGHV1-69-2*01 (SEQ ID NO:33) at this position.

hu10C12VHv2
10C12VHv2 retains all reverted framework substitutions at positions that are key for defining the Chothia canonical classes, are part of the Vernier zone, or localize to the VH/VL domain interface contribute to structural stability. Additionally, hu10C12VHv2 incorporates backmutations or substitution with most frequent residue at a given position and for optimization of deamination, oxidation, N-glycosylation, proteolysis and aggregation. Mutations in VHv2 that are not included in hu10C12VHv1 are:

Q1E: is a stability enhancing mutation to mitigate pyroglutamate formation potential to reduce N-term heterogeneity (Liu, supra.).

The rationales for selection of the positions indicated in Table 28 in the light chain variable region as candidates for substitution are as follows.

Kappa Light Chain Variable Regions

hu10C12VLv1
consists of the CDR-L1, L2, and L3 loops of 10C12-VL grafted onto the framework of CAB51297-VL. Additionally, hu10C12VLv1 also reverts all framework substitutions at positions that are key for defining the Chothia canonical classes, are part of the Vernier zone, or localize to the VH/VL domain interface contribute to structural stability.

G64S: is a back-mutation of a Vernier zone residue. G645 back-mutation preserves CDR packing.

hu10C12VLv2
retains all reverted framework substitutions at positions that are key for defining the Chothia canonical classes, are part of the Vernier zone, or localize to the VH/VL domain interface contribute to structural stability. Additionally, hu10C12VLv2 incorporates backmutations or substitution with most frequent residue at a given position and for optimization of deamination, oxidation, N-glycosylation, proteolysis and aggregation. Mutations in VLv2 that are not included in hu10C12VLv1 are:

V104L: is a germ-line aligning mutation. Leu is in human germ line gene IGKV2-28*01 & IGKJ2*01 (SEQ ID NO:37) at this position.

The designs based on these human frameworks were:

heavy chain variable regions
>hu10C12VHv1
(SEQ ID NO: 214)
QVQLVQSGAEVKKPGATVKISCKASGFNIKDDYMNWVQQAPGKGLEWIGW
IDPENGDTEYASKFQGRATMTADTSTDTAYMELSSLRSEDTAVYYCTTSN
GWGQGTLVTVSS
>hu10C12VHv2
(SEQ ID NO: 215)
EVQLVQSGAEVKKPGATVKISCKASGFNIKDDYMNWVQQAPGKGLEWIGW
IDPENGDTEYASKFQGRATMTADTSTDTAYMELSSLRSEDTAVYYCTTSN
GWGQGTLVTVSS
kappa light chain variable regions
>hu10C12VLv1
(SEQ ID NO: 216)
DIVMTQSPLSLPVTPGEPASISCRSSKSLLHSNGITYLYWYLQKPGQSPQ
LLIYQMSNLASGVPDRFSSSGSGTDFTLKISRVEAEDVGVYYCAQNLELP
LTFGGGTKVEIK
>hu10C12VLv2
(SEQ ID NO: 217)
DIVMTQSPLSLPVTPGEPASISCRSSKSLLHSNGITYLYWYLQKPGQSPQ
LLIYQMSNLASGVPDRFSSSGSGTDFTLKISRVEAEDVGVYYCAQNLELP
LTFGGGTKLEIK

Humanized sequences are generated using a two-stage PCR protocol that allows introduction of multiple mutations, deletions, and insertions using QuikChange site-directed mutagenesis [Wang, W. and Malcolm, B.A. (1999) BioTechniques 26:680-682).

### Example 10. Design of Humanized 12C4 Antibodies

The starting point or donor antibody for humanization was the mouse antibody 12C4. The heavy chain variable amino acid sequence of mature m12C4 is provided as SEQ ID NO:219. The light chain variable amino acid sequence of mature m12C4 is provided as SEQ ID NO:11. The heavy chain Kabat/Chothia Composite CDR1, CDR2, and CDR3 amino acid sequences are provided as SEQ ID NOs:8, 220, and 10, respectively. The light chain Kabat CDR1, CDR2, and CDR3 amino acid sequences are provided as SEQ ID NOs:12-14 respectively. Kabat numbering is used throughout.

The variable kappa (Vk) of 12C4 belongs to mouse Vk subgroup 2which corresponds to human Vk subgroup 2and the variable heavy (Vh) to mouse Vh subgroup 2c which corresponds to human Vh subgroup 1 [Kabat E. A., et al., (1991), Sequences of Proteins of Immunological Interest, Fifth Edition. NIH Publication No. 91-3242]. 16 residue Chothia CDR-L1 is similar to Chothia canonical class 4, 7 residue Chothia CDR-L2 is of Chothia canonical class 1, 9 residue Chothia CDR-L3 is similar to Chothia canonical class 1 [Martin ACR. (2010) Protein sequence and structure analysis of antibody variable domains. In: Kontermann R and Dübel S (eds). Antibody Engineering. Heidelberg, Germany: Springer International Publishing AG. [Martin, 2010]. 10 residue Chothia CDR-H1 is similar to Chothia canonical class 1, 17 residue Chothia CDR-H2 is similar to Chothia canonical class 2 [Martin, 2010]. 3 residue CDR-H3 has no canonical classes. A search was made over the protein sequences in the PDB database [Deshpande N, et al., (2005) Nucleic Acids Res. 33: D233-7] to find structures, which would provide a rough structural model of 12C4. The crystal structure of an antibody fab pdb code 5OBF [Vicentini, et al., 2017, unpublished] was used for both Vh and Vk structure since it had good resolution (1.92A°) and overall sequence similarity to 12C4 Vh and Vk, retaining the same canonical structures for the loops.

The frameworks of 12C4 VH share a high degree of sequence similarity with the corresponding regions of immunoglobulin heavy chain variable region [Homo sapiens] CAC20421 cloned by Arnold-Schild et al [Cancer Res. 60 (15), 4175-4178 (2000)]. Similarly, the frameworks of 12C4 VL share a high degree of sequence similarity with the corresponding regions of human antibody CAB51297 VL cloned by Capello et al (GenBank Ref. NO. CAB51297, submitted Jul. 20, 1999, unpublished). The variable light domain of 12C4 and CAB51297 VL also share identical lengths for the CDR-L1, L2 and L3 loops. Accordingly, the framework regions of CAC20421 VH and CAB51297 VL were chosen as the acceptor sequences for the CDRs of 12C4 VL. A model of the 12C4 CDRs grafted onto the respective human frameworks for VH and VL was built and used as a guidance for further backmutations.

Heavy and light chain variant sequences resulting from antibody humanization process were further aligned to human germ line sequences using IMGT Domain GapAlign tool to assess the humanness of the heavy and light chain as outlined by WHO INN committee guidelines. (WHO-INN: International nonproprietary names (INN) for biological and biotechnological substances (a review) (Internet) 2014. Available from: http://www.who.int/medicines/services/inn/BioRev2014.pdf) Residues were changed to align with corresponding human germ line sequence, where possible, to enhance humanness and to reduce potential immunogenicity. For humanized VLv1 and VLv2 variants, mutations were introduced to render the sequences more similar to human germ line gene IGKV2-28*01 & IGKJ2*01 (SEQ ID NO:37). For humanized VHv1 and VHv2 variants, mutations were introduced to render the sequences more similar to human germ line gene IGHV1-69-2*01 (SEQ ID NO:33)

Additional versions of hu12C4-VH and hu12C4-VL were designed to enable assessment of various framework residues for their contributions to antigen binding, thermostability, and immunogenicity, and for optimization of deamination, oxidation, N-glycosylation, proteolysis and aggregation. The positions considered for mutation include those that:
define the canonical CDR conformations (summarized in Martin, A. C. R. (2010) Protein sequence and structure analysis of antibody variable domains. In: Kontermann R and Dübel S (eds). Antibody Engineering. Heidelberg, Germany: Springer International Publishing AG.),
are within the Vernier zone (Foote J and Winter G. (1992) Antibody framework residues affecting the conformation of the hypervariable loops. J Mol Biol. 224(2):487-99.),
localize to the VH/VL domain interface (summarized in Léger O J P and Saldanha J. (2000) Preparation of recombinant antibodies from immune rodent spleens and the design of their humanisation by CDR grafting. In: Shepherd P and Dean C (eds). Monoclonal Antibodies: a Practical Approach. Oxford, UK: Oxford University Press.),
are susceptible to post-translational modifications, such as glycosylation or pyroglutamination,
are occupied by residues that are predicted to clash with CDRs, according to the model of 12C4 CDRs grafted onto VH and VL frameworks, or
are occupied by residues that are rare among sequenced human antibodies, where either the parental mouse 12C4 residue or some other residue is much more prevalent within human antibody repertoire.

Alignments of the murine 12C4 and various humanized antibodies are shown for the light chain variable regions (Table 34 and FIG. 10), and heavy chain variable regions (Table 33 and FIG. 9).

2 humanized heavy chain variable region variants and 2 humanized light chain variable region variants were constructed containing different permutations of substitutions: hu12C4VHv1 or hu12C4VHv2, (SEQ ID NOs:221-222, respectively); and hu12C4VLv1 or hu12C4VLv2, (SEQ ID NOs:223-224, respectively) (Tables 33 and 34). The exemplary humanized Vk and Vh designs, with backmutations and other mutations based on selected human frameworks, are shown in Tables 33and 34, respectively. The bolded areas in Tables 33 and 34 indicate the CDRs as defined by Kabat/Chothia Composite. A “−” in the columns in Tables 33 and 34 indicates no residue at the indicated position. SEQ ID NOs:221-222, and SEQ ID NOs:223-224 contain backmutations and other mutations as shown in Table 35. The amino acids at positions in hu12C4VHv1 and hu12C4VHv2 are listed in Table 36. The amino acids at positions in hu12C4VLv1 and hu12C4VLv2 are listed in Table 37.

The percentage humanness for humanized VH chains hu12C4VHv1 and hu12C4VHv2 (SEQ ID NOs:221-222, respectively) with respect to the most similar human germline gene IGHV1-69-2*01 (SEQ ID NO:33), and for humanized VL chains hu12C4VLv1 and hu12C4VLv2 (SEQ ID NOs:223-224, respectively) with respect to the most similar human germline gene IGKV2-28*01 & IGKJ2*01 (SEQ ID NO:37), is shown in Table 38.

TABLE 33

| | | | | IMGT# | | | |
|---|---|---|---|---|---|---|---|
| | | | Mouse 12C4 VH | IGHV1-69-2*01 | CAC20421 VH | Hu12C4VHv1 | Hu12C4VHv2 |
| Kabat | Linear | FR or | (SEQ ID | (SEQ ID | (SEQ ID | (SEQ ID | (SEQ ID |
| residue # | residue # | CDR | NO: 219) | NO: 33) | NO: 218) | NO: 221) | NO: 222) |
| 1 | 1 | Fr1 | E | E | Q | Q | E |
| 2 | 2 | Fr1 | V | V | V | V | V |
| 3 | 3 | Fr1 | Q | Q | Q | Q | Q |
| 4 | 4 | Fr1 | L | L | L | L | L |
| 5 | 5 | Fr1 | Q | V | V | V | V |
| 6 | 6 | Fr1 | Q | Q | Q | Q | Q |
| 7 | 7 | Fr1 | S | S | S | S | S |
| 8 | 8 | Fr1 | G | G | G | G | G |
| 9 | 9 | Fr1 | A | A | A | A | A |
| 10 | 10 | Fr1 | E | E | E | E | E |
| 11 | 11 | Fr1 | L | V | V | V | V |
| 12 | 12 | Fr1 | V | K | K | K | K |
| 13 | 13 | Fr1 | R | K | K | K | K |
| 14 | 14 | Fr1 | P | P | P | P | P |
| 15 | 15 | Fr1 | G | G | G | G | G |
| 16 | 16 | Fr1 | A | A | A | A | A |
| 17 | 17 | Fr1 | S | T | T | T | T |
| 18 | 18 | Fr1 | V | V | V | V | V |
| 19 | 19 | Fr1 | K | K | K | K | K |
| 20 | 20 | Fr1 | L | I | I | I | I |
| 21 | 21 | Fr1 | S | S | S | S | S |
| 22 | 22 | Fr1 | C | C | C | C | C |
| 23 | 23 | Fr1 | T | K | K | K | K |
| 24 | 24 | Fr1 | A | V | V | V | V |
| 25 | 25 | Fr1 | S | S | S | S | S |
| 26 | 26 | CDR-H1 | G | G | G | G | G |
| 27 | 27 | CDR-H1 | F | Y | Y | F | F |
| 28 | 28 | CDR-H1 | N | T | T | N | N |
| 29 | 29 | CDR-H1 | I | F | F | I | I |
| 30 | 30 | CDR-H1 | K | T | T | K | K |
| 31 | 31 | CDR-H1 | D | D | D | D | D |
| 32 | 32 | CDR-H1 | D | Y | Y | D | D |
| 33 | 33 | CDR-H1 | Y | Y | Y | Y | Y |
| 34 | 34 | CDR-H1 | M | M | M | M | M |
| 35 | 35 | CDR-H1 | N | H | H | N | N |
| 35A | | CDR-H1 | — | — | — | — | — |
| 35B | | CDR-H1 | — | — | — | — | — |
| 36 | 36 | Fr2 | W | W | W | W | W |
| 37 | 37 | Fr2 | V | V | V | V | V |
| 38 | 38 | Fr2 | R | Q | Q | Q | Q |
| 39 | 39 | Fr2 | Q | Q | Q | Q | Q |
| 40 | 40 | Fr2 | R | A | A | A | A |
| 41 | 41 | Fr2 | P | P | P | P | P |
| 42 | 42 | Fr2 | E | G | G | G | G |
| 43 | 43 | Fr2 | R | K | K | K | K |
| 44 | 44 | Fr2 | G | G | G | G | G |
| 45 | 45 | Fr2 | L | L | L | L | L |
| 46 | 46 | Fr2 | E | E | E | E | E |
| 47 | 47 | Fr2 | W | W | W | W | W |
| 48 | 48 | Fr2 | I | M | M | M | I |
| 49 | 49 | Fr2 | G | G | G | G | G |
| 50 | 50 | CDR-H2 | W | L | L | W | W |
| 51 | 51 | CDR-H2 | I | V | V | I | I |
| 52 | 52 | CDR-H2 | D | D | D | D | D |
| 52A | 53 | CDR-H2 | P | P | P | P | P |
| 52B | | CDR-H2 | — | — | — | — | — |
| 52C | | CDR-H2 | — | — | — | — | — |
| 53 | 54 | CDR-H2 | E | E | E | E | E |
| 54 | 55 | CDR-H2 | N | D | D | N | N |
| 55 | 56 | CDR-H2 | G | G | G | G | G |
| 56 | 57 | CDR-H2 | D | E | E | D | D |
| 57 | 58 | CDR-H2 | T | T | T | T | T |
| 58 | 59 | CDR-H2 | A | I | I | A | A |
| 59 | 60 | CDR-H2 | Y | Y | Y | Y | Y |
| 60 | 61 | CDR-H2 | A | A | A | A | A |
| 61 | 62 | CDR-H2 | S | E | E | S | S |
| 62 | 63 | CDR-H2 | K | K | K | K | K |
| 63 | 64 | CDR-H2 | F | F | F | F | F |
| 64 | 65 | CDR-H2 | Q | Q | Q | Q | Q |
| 65 | 66 | CDR-H2 | G | G | G | G | G |
| 66 | 67 | Fr3 | K | R | R | R | R |
| 67 | 68 | Fr3 | A | V | V | V | V |
| 68 | 69 | Fr3 | T | T | T | T | T |
| 69 | 70 | Fr3 | M | I | I | I | I |
| 70 | 71 | Fr3 | T | T | T | T | T |
| 71 | 72 | Fr3 | A | A | A | A | A |
| 72 | 73 | Fr3 | D | D | D | D | D |
| 73 | 74 | Fr3 | T | T | T | T | T |
| 74 | 75 | Fr3 | S | S | S | S | S |
| 75 | 76 | Fr3 | S | T | T | T | T |
| 76 | 77 | Fr3 | N | D | D | D | D |
| 77 | 78 | Fr3 | T | T | T | T | T |
| 78 | 79 | Fr3 | A | A | A | A | A |
| 79 | 80 | Fr3 | Y | Y | Y | Y | Y |
| 80 | 81 | Fr3 | L | M | M | M | M |
| 81 | 82 | Fr3 | Q | E | E | E | E |
| 82 | 83 | Fr3 | F | L | L | L | L |
| 82A | 84 | Fr3 | S | S | S | S | S |
| 82B | 85 | Fr3 | S | S | S | S | S |
| 82C | 86 | Fr3 | L | L | L | L | L |
| 83 | 87 | Fr3 | T | R | R | R | R |
| 84 | 88 | Fr3 | S | S | S | S | S |
| 85 | 89 | Fr3 | E | E | E | E | E |
| 86 | 90 | Fr3 | D | D | D | D | D |
| 87 | 91 | Fr3 | S | T | T | T | T |
| 88 | 92 | Fr3 | A | A | A | A | A |
| 89 | 93 | Fr3 | V | V | V | V | V |
| 90 | 94 | Fr3 | Y | Y | Y | Y | Y |
| 91 | 95 | Fr3 | Y | Y | Y | Y | Y |
| 92 | 96 | Fr3 | C | C | C | C | C |
| 93 | 97 | Fr3 | T | A | A | A | T |
| 94 | 98 | Fr3 | T | T | R | R | T |
| 95 | 99 | CDR-H3 | S | — | I | S | S |
| 96 | | CDR-H3 | — | — | P | — | — |
| 97 | | CDR-H3 | — | — | L | — | — |
| 98 | | CDR-H3 | — | — | F | — | — |
| 99 | | CDR-H3 | — | — | G | — | — |
| 100 | | CDR-H3 | — | — | R | — | — |
| 100A | | CDR-H3 | — | — | — | — | — |
| 100B | | CDR-H3 | — | — | — | — | — |
| 100C | | CDR-H3 | — | — | — | — | — |
| 100D | | CDR-H3 | — | — | — | — | — |
| 100E | | CDR-H3 | — | — | — | — | — |
| 100F | | CDR-H3 | — | — | — | — | — |
| 100G | | CDR-H3 | — | — | — | — | — |
| 100H | | CDR-H3 | — | — | — | — | — |
| 100I | | CDR-H3 | — | — | — | — | — |
| 100J | | CDR-H3 | — | — | — | — | — |
| 100K | | CDR-H3 | — | — | — | — | — |
| 101 | 100 | CDR-H3 | N | Q | D | N | N |
| 102 | 101 | CDR-H3 | G | H | H | G | G |
| 103 | 102 | Fr4 | W | W | W | W | W |
| 104 | 103 | Fr4 | G | G | G | G | G |
| 105 | 104 | Fr4 | Q | Q | Q | Q | Q |
| 106 | 105 | Fr4 | G | G | G | G | G |
| 107 | 106 | Fr4 | T | T | T | T | T |
| 108 | 107 | Fr4 | L | L | L | L | L |
| 109 | 108 | Fr4 | V | V | V | V | V |
| 110 | 109 | Fr4 | T | T | T | T | T |
| 111 | 110 | Fr4 | V | V | V | V | V |
| 112 | 111 | Fr4 | S | S | S | S | S |
| 113 | 112 | Fr4 | A | S | S | S | S |

TABLE 34

| Kabat | Linear | | | IGKV2-28*01 & | CAB51297 VL | Hu12C4VLv1 | Hu12C4VLv2 |
|---|---|---|---|---|---|---|---|
| residue | residue | FR or | Mouse 12C4 VL | IGKJ2*01 | (SEQ ID | (SEQ ID | (SEQ ID |
| # | # | CDR | (SEQ ID NO: 11) | (SEQ ID NO: 37) | NO: 35) | NO: 223) | NO: 224) |
| 1 | 1 | Fr1 | D | D | D | D | D |
| 2 | 2 | Fr1 | I | I | I | I | I |
| 3 | 3 | Fr1 | V | V | V | V | V |
| 4 | 4 | Fr1 | M | M | M | M | M |
| 5 | 5 | Fr1 | T | T | T | T | T |
| 6 | 6 | Fr1 | Q | Q | Q | Q | Q |
| 7 | 7 | Fr1 | A | S | S | S | S |
| 8 | 8 | Fr1 | A | P | P | P | P |
| 9 | 9 | Fr1 | F | L | L | L | L |
| 10 | 10 | Fr1 | S | S | S | S | S |
| 11 | 11 | Fr1 | N | L | L | L | L |
| 12 | 12 | Fr1 | P | P | P | P | P |
| 13 | 13 | Fr1 | V | V | V | V | V |
| 14 | 14 | Fr1 | T | T | T | T | T |
| 15 | 15 | Fr1 | L | P | P | P | P |
| 16 | 16 | Fr1 | G | G | G | G | G |
| 17 | 17 | Fr1 | T | E | E | E | E |
| 18 | 18 | Fr1 | S | P | P | P | P |
| 19 | 19 | Fr1 | A | A | A | A | A |
| 20 | 20 | Fr1 | S | S | S | S | S |
| 21 | 21 | Fr1 | I | I | I | I | I |
| 22 | 22 | Fr1 | S | S | S | S | S |
| 23 | 23 | Fr1 | C | C | C | C | C |
| 24 | 24 | CDR-L1 | R | R | R | R | R |
| 25 | 25 | CDR-L1 | S | S | S | S | S |
| 26 | 26 | CDR-L1 | S | S | S | S | S |
| 27 | 27 | CDR-L1 | K | Q | Q | K | K |
| 27A | 28 | CDR-L1 | S | S | S | S | S |
| 27B | 29 | CDR-L1 | L | L | L | L | L |
| 27C | 30 | CDR-L1 | L | L | L | L | L |
| 27D | 31 | CDR-L1 | H | H | H | H | H |
| 27E | 32 | CDR-L1 | S | S | S | S | S |
| 27F | | CDR-L1 | — | — | — | — | — |
| 28 | 33 | CDR-L1 | N | N | N | N | N |
| 29 | 34 | CDR-L1 | G | G | G | G | G |
| 30 | 35 | CDR-L1 | I | Y | Y | I | I |
| 31 | 36 | CDR-L1 | T | N | N | T | T |
| 32 | 37 | CDR-L1 | Y | Y | Y | Y | Y |
| 33 | 38 | CDR-L1 | L | L | L | L | L |
| 34 | 39 | CDR-L1 | Y | D | D | Y | Y |
| 35 | 40 | Fr2 | W | W | W | W | W |
| 36 | 41 | Fr2 | Y | Y | Y | Y | Y |
| 37 | 42 | Fr2 | L | L | L | L | L |
| 38 | 43 | Fr2 | Q | Q | Q | Q | Q |
| 39 | 44 | Fr2 | K | K | K | K | K |
| 40 | 45 | Fr2 | P | P | P | P | P |
| 41 | 46 | Fr2 | G | G | G | G | G |
| 42 | 47 | Fr2 | Q | Q | Q | Q | Q |
| 43 | 48 | Fr2 | S | S | S | S | S |
| 44 | 49 | Fr2 | P | P | P | P | P |
| 45 | 50 | Fr2 | Q | Q | Q | Q | Q |
| 46 | 51 | Fr2 | L | L | L | L | L |
| 47 | 52 | Fr2 | L | L | L | L | L |
| 48 | 53 | Fr2 | I | I | I | I | I |
| 49 | 54 | Fr2 | Y | Y | Y | Y | Y |
| 50 | 55 | CDR-L2 | Q | L | L | Q | Q |
| 51 | 56 | CDR-L2 | M | G | G | M | M |
| 52 | 57 | CDR-L2 | S | S | S | S | S |
| 53 | 58 | CDR-L2 | N | N | N | N | N |
| 54 | 59 | CDR-L2 | L | R | R | L | L |
| 55 | 60 | CDR-L2 | A | A | A | A | A |
| 56 | 61 | CDR-L2 | S | S | S | S | S |
| 57 | 62 | Fr3 | G | G | G | G | G |
| 58 | 63 | Fr3 | V | V | V | V | V |
| 59 | 64 | Fr3 | P | P | P | P | P |
| 60 | 65 | Fr3 | D | D | D | D | D |
| 61 | 66 | Fr3 | R | R | R | R | R |
| 62 | 67 | Fr3 | F | F | F | F | F |
| 63 | 68 | Fr3 | S | S | S | S | S |
| 64 | 69 | Fr3 | S | G | G | G | S |
| 65 | 70 | Fr3 | S | S | S | S | S |
| 66 | 71 | Fr3 | G | G | G | G | G |
| 67 | 72 | Fr3 | S | S | S | S | S |
| 68 | 73 | Fr3 | G | G | G | G | G |
| 69 | 74 | Fr3 | T | T | T | T | T |
| 70 | 75 | Fr3 | D | D | D | D | D |
| 71 | 76 | Fr3 | F | F | F | F | F |
| 72 | 77 | Fr3 | T | T | T | T | T |
| 73 | 78 | Fr3 | L | L | L | L | L |
| 74 | 79 | Fr3 | R | K | K | K | K |
| 75 | 80 | Fr3 | I | I | I | I | I |
| 76 | 81 | Fr3 | S | S | S | S | S |
| 77 | 82 | Fr3 | R | R | R | R | R |
| 78 | 83 | Fr3 | V | V | V | V | V |
| 79 | 84 | Fr3 | E | E | E | E | E |
| 80 | 85 | Fr3 | A | A | A | A | A |
| 81 | 86 | Fr3 | E | E | E | E | E |
| 82 | 87 | Fr3 | D | D | D | D | D |
| 83 | 88 | Fr3 | V | V | V | V | V |
| 84 | 89 | Fr3 | G | G | G | G | G |
| 85 | 90 | Fr3 | V | V | V | V | V |
| 86 | 91 | Fr3 | Y | Y | Y | Y | Y |
| 87 | 92 | Fr3 | Y | Y | Y | Y | Y |
| 88 | 93 | Fr3 | C | C | C | C | C |
| 89 | 94 | CDR-L3 | A | M | M | A | A |
| 90 | 95 | CDR-L3 | Q | Q | Q | Q | Q |
| 91 | 96 | CDR-L3 | N | A | A | N | N |
| 92 | 97 | CDR-L3 | L | L | L | L | L |
| 93 | 98 | CDR-L3 | E | Q | Q | E | E |
| 94 | 99 | CDR-L3 | L | T | T | L | L |
| 95 | 100 | CDR-L3 | P | P | P | P | P |
| 95A | | CDR-L3 | — | — | — | — | — |
| 95B | | CDR-L3 | — | — | — | — | — |
| 95C | | CDR-L3 | — | — | — | — | — |
| 95D | | CDR-L3 | — | — | — | — | — |
| 95E | | CDR-L3 | — | — | — | — | — |
| 95F | | CDR-L3 | — | — | — | — | — |
| 96 | 101 | CDR-L3 | L | Y | L | L | L |
| 97 | 102 | CDR-L3 | T | T | T | T | T |
| 98 | 103 | Fr4 | F | F | F | F | F |
| 99 | 104 | Fr4 | G | G | G | G | G |
| 100 | 105 | Fr4 | A | Q | G | G | G |
| 101 | 106 | Fr4 | G | G | G | G | G |
| 102 | 107 | Fr4 | T | T | T | T | T |
| 103 | 108 | Fr4 | K | K | K | K | K |
| 104 | 109 | Fr4 | L | L | V | V | L |
| 105 | 110 | Fr4 | E | E | E | E | E |
| 106 | 111 | Fr4 | L | I | I | I | I |
| 106A | | Fr4 | — | — | — | — | — |
| 107 | 112 | Fr4 | K | K | K | K | K |

TABLE 35
V_{H}, V_{L} Backmutations and Other Mutations for Humanized 12C4

| | | Changes from Acceptor Framework |
|---|---|---|
| | | (or CDR) Residues (based on |
| V_{H} or V_{L} Variant | V_{H} or V_{L} Exon Acceptor Sequence | Kabat/Chothia Composite CDRs) |
| Hu12C4VHv1 | GenBank Acc. # CAC20421 VH | None |
| (SEQ ID NO: 221) | (SEQ ID NO: 218) | |
| Hu12C4VHv2 | GenBank Acc. # CAC20421 VH | H1, H48, H93, H94 |
| (SEQ ID NO: 222) | (SEQ ID NO: 218) | |
| | IMGT# IGHV1-69-2*01 | |
| | (SEQ ID NO: 33) | |
| Hu12C4VLv1 | GenBank Acc. # CAB51297 VL | None |
| (SEQ ID NO: 223) | (SEQ ID NO: 35) | |
| Hu12C4VLv2 | GenBank Acc. # CAB51297 VL | L64, L104 |
| (SEQ ID NO: 224) | (SEQ ID NO: 35) | |
| | IGKV2-28*01 & IGKJ2*01 | |
| | (SEQ ID NO: 37) | |

TABLE 36
Kabat Numbering of Framework (or CDR) Residues (based on Kabat/Chothia
Composite CDRs) for Backmutations and Other Mutations in Heavy Chains
of Humanized 12C4 Antibodies

| | | IMGT# | | | |
|---|---|---|---|---|---|
| | CAC20421 VH | ICHV1-69-2*01 | Mouse 12C4 VH | Hu12C4Hv1 | Hu12C4VHv2 |
| | (SEQ ID | (SEQ ID | (SEQ ID | (SEQ ID | (SEQ ID |
| Residue | NO: 218) | NO: 33) | NO: 219) | NO: 221) | NO: 222) |
| H1 | Q | E | E | Q | E |
| H48 | M | M | I | M | I |
| H93 | A | A | T | A | T |
| H94 | R | T | T | R | T |

TABLE 37
Kabat Numbering of Framework Residues (based on Kabat/Chothia
Composite CDRs) for Backmutations and Other Mutations in Light
Chains of Humanized 12C4 Antibodies

| | | IGKV2-28*01 & | | | |
|---|---|---|---|---|---|
| | CAB52197 VL | IGKJ2*01 | Mouse 12C4 VL | Hu12C4VLv1 | Hu12C4VLv2 |
| | (SEQ ID | (SEQ ID | (SEQ ID | (SEQ ID | (SEQ ID |
| Residue | NO: 35) | NO: 37) | NO: 11) | NO: 223) | NO: 224) |
| L64 | G | G | S | G | S |
| L104 | V | L | L | V | L |

TABLE 38
Percentage Humanness of Heavy and Light
Chains of Humanized 12C4 Antibodies

| V_{H} or V_{L} Variant | % Humanness |
|---|---|
| Hu12C4VHv1 (SEQ ID NO: 221) | 86.6% |
| Hu12C4VHv2 (SEQ ID NO: 222) | 85.7% |
| Hu12C4VLv1 (SEQ ID NO: 223) | 89.0% |
| Hu12C4VLv2 (SEQ ID NO: 224) | 88.0% |

Positions at which canonical, vernier, or interface residues differ between mouse and human acceptor sequences are candidates for substitution. Examples of canonical/CDR interacting residues include Kabat residue H94 in Table 33. Examples of vernier residues include Kabat residues H48, H93, and H94 in Table 33 and L64 in Table 34. Examples of interface/packing (VH+VL) residues include Kabat residue H93 in Table 33.

The rationales for selection of the positions indicated in Table 33 in the heavy chain variable region as candidates for substitution are as follows.

Heavy Chain Variable Regions

hu12C4VHv1
consists of the CDR-H1, H2, and H3 loops of 12C4-VH grafted onto the framework of CAC20421.

hu12C4VHv2
reverts all framework substitutions at positions that are key for defining the Chothia canonical classes, are part of the Vernier zone, or localize to the VH/VL domain interface or contribute to structural stability. 12C4-VH_v2 incorporates backmutations or substitution with most frequent residue at a given position.

Q1E: is a stability enhancing mutation to mitigate pyroglutamate formation potential to reduce N-term heterogeneity (Liu, supra.)

M48I: is a backmutation of a Vernier zone residue.

A93T: is a backmutation of a VH/VL interface residue, and is backmutated to preserve the interface.

R94T: is a backmutation of a Vernier zone residue. R94T is also a germ line-aligning mutation. Thr is in human germ line gene IGHV1-69-2*01 (SEQ ID NO:33) at this position.

The rationales for selection of the positions indicated in Table 34 in the light chain variable region as candidates for substitution are as follows.

Kappa Light Chain Variable Regions

hu12C4VLv1
consists of the CDR-L1, L2, and L3 loops of 12C4-VL grafted onto the framework of CAB51297 VL along with reverting all framework substitutions at positions that are key for defining the Chothia canonical classes, are part of the Vernier zone, or locate to the VH/VL domain interface. For hu12C4VLv1 no position other than G64 meets this criterion, therefore no back mutation is made. Based on homology model analysis Gly at position 64 might be tolerated, therefore no change is made at position 64 in 12C4VLv1, substitution with Ser is tried in hu12C4VLv2.

Hu12C4VLv2
hu12C4VLv2 includes substitutions that contribute to structural stability or increases the humanness of the antibody.

G64S: is a back-mutation of a Vernier zone residue. G645 back-mutation preserves CDR packing.

V104L: is a germ-line aligning mutation. Leu is in human germ line gene IGKV2-28*01 & IGKJ2*01 (SEQ ID NO:37) at this position.

The designs based on these human frameworks were:

heavy chain variable regions
>hu12C4VHv1
(SEQ ID NO: 221)
QVQLVQSGAEVKKPGATVKISCKVSGFNIKDDYMNWVQQAPGKGLEWMGW
IDPENGDTAYASKFQGRVTITADTSTDTAYMELSSLRSEDTAVYYCARSN
GWGQGTLVTVSS
>hu12C4VHv2
(SEQ ID NO: 222)
EVQLVQSGAEVKKPGATVKISCKVSGFNIKDDYMNWVQQAPGKGLEWIGW
IDPENGDTAYASKFQGRVTITADTSTDTAYMELSSLRSEDTAVYYCTTSN
GWGQGTLVTVSS
kappa light chain variable regions
>hu12C4VLv1
(SEQ ID NO: 223)
DIVMTQSPLSLPVTPGEPASISCRSSKSLLHSNGITYLYWYLQKPGQSPQ
LLIYQMSNLASGVPDRFSGSGSGTDFTLKISRVEAEDVGVYYCAQNLELP
LTFGGGTKVEIK
>hu12C4VLv2
(SEQ ID NO: 224)
DIVMTQSPLSLPVTPGEPASISCRSSKSLLHSNGITYLYWYLQKPGQSPQ
LLIYQMSNLASGVPDRFSSSGSGTDFTLKISRVEAEDVGVYYCAQNLELP
LTFGGGTKLEIK

Humanized sequences are generated using a two-stage PCR protocol that allows introduction of multiple mutations, deletions, and insertions using QuikChange site-directed mutagenesis [Wang, W. and Malcolm, B. A. (1999) BioTechniques 26:680-682).

### Example 11. Design of Humanized 17C12 Antibodies

The starting point or donor antibody for humanization was the mouse antibody 17C12. The heavy chain variable amino acid sequence of mature m17C12 is provided as SEQ ID NO:225. The light chain variable amino acid sequence of mature m17C12 is provided as SEQ ID NO:228. The heavy chain Kabat/Chothia Composite CDR1, CDR2, and CDR3 amino acid sequences are provided as SEQ ID NOs:226, 227, and 10, respectively. The light chain Kabat CDR1, CDR2, and CDR3 amino acid sequences are provided as SEQ ID NOs:229-231 respectively. Kabat numbering is used throughout.

The variable kappa (Vk) of 17C12 belongs to mouse Vk subgroup 2 which corresponds to human Vk subgroup 3 and the variable heavy (Vh) to mouse Vh subgroup 2c which corresponds to human Vh subgroup 1 [Kabat E.A., et al., (1991), Sequences of Proteins of Immunological Interest, Fifth Edition. NIH Publication No. 91-3242]. 16 residue Chothia CDR-L1 is similar to Chothia canonical class 4, 7 residue Chothia CDR-L2 is of Chothia canonical class 1, 9 residue Chothia CDR-L3 is similar to Chothia canonical class 1 [Martin ACR. (2010) Protein sequence and structure analysis of antibody variable domains. In: Kontermann R and Dübel S (eds). Antibody Engineering. Heidelberg, Germany: Springer International Publishing AG. [Martin, 2010]. 10 residue Chothia CDR-H1 is similar to Chothia canonical class 1, 17 residue Chothia CDR-H2 is similar to Chothia canonical class 2 [Martin, 2010]. 3 residue CDR-H3 has no canonical classes. A search was made over the protein sequences in the PDB database [Deshpande N, et al., (2005) Nucleic Acids Res. 33: D233-7] to find structures, which would provide a rough structural model of 17C12. The crystal structure of an antibody fab pdb code 3PP3 [Niederfellner, et al., 2011, Blood 118. 358-367] was used for both Vh and Vk structure since it had good resolution (2.51A°) and overall sequence similarity to 17C12 Vh and Vk, retaining the same canonical structures for the loops.

The frameworks of 17C12 VH share a high degree of sequence similarity with the corresponding regions of human antibody CAC20421, cloned by Arnold-Schild, et al [Cancer Res. 60 (15), 4175-4178 (2000)]. The variable heavy domains of 17C12 and CAC20421also share identical lengths for the CDR-H1, H2 loops. Similarly, the frameworks of 17C12 VL share a high degree of sequence similarity with the corresponding regions of human antibody QD016713 VL cloned by Cho et al [Cell Rep 28 (4), 909-922.e6 (2019)]. The variable light domain of 17C12 and QD016713 antibody also share identical lengths for the CDR-L1, L2 and L3 loops. Accordingly, the framework regions of CAC20421 VH and QD016713 VL were chosen as the acceptor sequences for the CDRs of 17C12. A model of the 17C12 CDRs grafted onto the respective human frameworks for VH and VL was built and used as a guidance for further backmutations.

Heavy and light chain variant sequences resulting from antibody humanization process were further aligned to human germ line sequences using IMGT Domain GapAlign tool to assess the humanness of the heavy and light chain as outlined by WHO INN committee guidelines. (WHO-INN: International nonproprietary names (INN) for biological and biotechnological substances (a review) (Internet) 2014. Available from: http://www.who.int/medicines/services/inn/BioRev2014.pdf) Residues were changed to align with corresponding human germ line sequence, where possible, to enhance humanness and to reduce potential immunogenicity. For humanized VLv2 and VLv2 variants, mutations were introduced to render the sequences more similar to human germ line IGKV2-29*02 & IGKJ4*01 (SEQ ID NO:239) For humanized VHv1 and VHv variants, mutations were introduced to render the sequences more similar to human germ line gene IGHV1-69-2*01 (SEQ ID NO:33).

Additional versions of hu17C12-VH and hu17C12-VL were designed to enable assessment of various framework residues for their contributions to antigen binding, thermostability, and immunogenicity, and for optimization of deamination, oxidation, N-glycosylation, proteolysis and aggregation. The positions considered for mutation include those that:
define the canonical CDR conformations (summarized in Martin, A. C. R. (2010) Protein sequence and structure analysis of antibody variable domains. In: Kontermann R and Dübel S (eds). Antibody Engineering. Heidelberg, Germany: Springer International Publishing AG.),
are within the Vernier zone (Foote J and Winter G. (1992) Antibody framework residues affecting the conformation of the hypervariable loops. J Mol Biol. 224(2):487-99.),
localize to the VH/VL domain interface (summarized in Léger O J P and Saldanha J. (2000) Preparation of recombinant antibodies from immune rodent spleens and the design of their humanisation by CDR grafting. In: Shepherd P and Dean C (eds). Monoclonal Antibodies: a Practical Approach. Oxford, UK: Oxford University Press.),
are susceptible to post-translational modifications, such as glycosylation or pyroglutamination,
are occupied by residues that are predicted to clash with CDRs, according to the model of 17C12 CDRs grafted onto VH and VL frameworks, or
are occupied by residues that are rare among sequenced human antibodies, where either the parental mouse 17C12 residue or some other residue is much more prevalent within human antibody repertoire.

Alignments of the murine 17C12 and various humanized antibodies are shown for the light chain variable regions (Table 40 and FIG. 12), and heavy chain variable regions (Table 39 and FIG. 11).

2 humanized heavy chain variable region variants and 2 humanized light chain variable region variants were constructed containing different permutations of substitutions: hu17C12VHv1 or hu17C12VHv2, (SEQ ID NOs:232-233, respectively); and hu17C12VLv1 or hu17C12VLv2 (SEQ ID NOs:234-235, respectively) (Tables 39 and 40). The exemplary humanized Vk and Vh designs, with backmutations and other mutations based on selected human frameworks, are shown in Tables 39 and 40, respectively. The bolded areas in Tables 39 and 40 indicate the CDRs as defined by Kabat/Chothia Composite. A “−” in the columns in Tables 39 and 40 indicates no residue at the indicated position. SEQ ID NOs:232-233, and SEQ ID NOs:234-235 contain backmutations and other mutations as shown in Table 41. The amino acids at positions in hu17C12VHv1 and hu17C12VHv2 are listed in Table 42. The amino acids at positions in hu17C12VLv1 and hu17C12VLv2 are listed in Table 43.

The percentage humanness for humanized VH chains hu17C12VHv1 and hu17C12VHv2 (SEQ ID NOs:232-233, respectively) with respect to the most similar human germline gene IGHV1-69-2*01 (SEQ ID NO:33), and for humanized VL chains hu17C12VLv1 and hu17C12VLv2 (SEQ ID NOs:234-235, respectively) with respect to the most similar human germline gene IGKV2-29*02 & IGKJ4*01 (SEQ ID NO:239), is shown in Table 44.

TABLE 39

| | | | | IMGT# | CAC20421- | | |
|---|---|---|---|---|---|---|---|
| | | | Mouse 17C12 VH | IGHV1-69-2*01 | VH_huFrwk | Hu17C12VHv1 | Hu17C12VHv2 |
| Kabat | Linear | FR or | (SEQ ID | (SEQ ID | (SEQ ID: | (SEQ ID | (SEQ ID |
| residue # | residue # | CDR | NO: 225) | NO: 33) | 218) | NO: 232) | NO: 233) |
| 1 | 1 | Fr1 | K | E | Q | Q | E |
| 2 | 2 | Fr1 | I | V | V | I | I |
| 3 | 3 | Fr1 | Q | Q | Q | Q | Q |
| 4 | 4 | Fr1 | L | L | L | L | L |
| 5 | 5 | Fr1 | Q | V | V | V | V |
| 6 | 6 | Fr1 | Q | Q | Q | Q | Q |
| 7 | 7 | Fr1 | S | S | S | S | S |
| 8 | 8 | Fr1 | G | G | G | G | G |
| 9 | 9 | Fr1 | A | A | A | A | A |
| 10 | 10 | Fr1 | E | E | E | E | E |
| 11 | 11 | Fr1 | L | V | V | V | V |
| 12 | 12 | Fr1 | V | K | K | K | K |
| 13 | 13 | Fr1 | R | K | K | K | K |
| 14 | 14 | Fr1 | P | P | P | P | P |
| 15 | 15 | Fr1 | G | G | G | G | G |
| 16 | 16 | Fr1 | A | A | A | A | A |
| 17 | 17 | Fr1 | S | T | T | T | T |
| 18 | 18 | Fr1 | V | V | V | V | V |
| 19 | 19 | Fr1 | K | K | K | K | K |
| 20 | 20 | Fr1 | L | I | I | I | I |
| 21 | 21 | Fr1 | S | S | S | S | S |
| 22 | 22 | Fr1 | C | C | C | C | C |
| 23 | 23 | Fr1 | T | K | K | K | K |
| 24 | 24 | Fr1 | A | V | V | A | A |
| 25 | 25 | Fr1 | S | S | S | S | S |
| 26 | 26 | CDR-H1 | A | G | G | A | A |
| 27 | 27 | CDR-H1 | F | Y | Y | F | F |
| 28 | 28 | CDR-H1 | N | T | T | N | N |
| 29 | 29 | CDR-H1 | I | | F | I | I |
| 30 | 30 | CDR-H1 | K | T | T | K | K |
| 31 | 31 | CDR-H1 | D | D | D | D | D |
| 32 | 32 | CDR-H1 | D | Y | Y | D | D |
| 33 | 33 | CDR-H1 | Y | Y | Y | Y | Y |
| 34 | 34 | CDR-H1 | M | M | M | M | M |
| 35 | 35 | CDR-H1 | N | H | H | N | N |
| 35A | | CDR-H1 | — | — | — | — | — |
| 35B | | CDR-H1 | — | — | — | — | — |
| 36 | 36 | Fr2 | W | W | W | W | W |
| 37 | 37 | Fr2 | V | V | V | V | V |
| 38 | 38 | Fr2 | K | Q | Q | Q | Q |
| 39 | 39 | Fr2 | Q | Q | Q | Q | Q |
| 40 | 40 | Fr2 | R | A | A | A | A |
| 41 | 41 | Fr2 | P | P | P | P | P |
| 42 | 42 | Fr2 | E | G | G | G | G |
| 43 | 43 | Fr2 | R | K | K | K | K |
| 44 | 44 | Fr2 | G | G | G | G | G |
| 45 | 45 | Fr2 | L | L | L | L | L |
| 46 | 46 | Fr2 | E | E | E | E | E |
| 47 | 47 | Fr2 | W | W | W | W | W |
| 48 | 48 | Fr2 | I | M | M | I | I |
| 49 | 49 | Fr2 | G | G | G | G | G |
| 50 | 50 | CDR-H2 | W | L | L | W | W |
| 51 | 51 | CDR-H2 | I | V | V | I | I |
| 52 | 52 | CDR-H2 | D | D | D | D | D |
| 52A | 53 | CDR-H2 | P | P | P | P | P |
| 52B | | CDR-H2 | — | — | — | — | — |
| 52C | | CDR-H2 | — | — | — | — | — |
| 53 | 54 | CDR-H2 | E | E | E | E | E |
| 54 | 55 | CDR-H2 | N | D | D | N | N |
| 55 | 56 | CDR-H2 | G | G | G | G | G |
| 56 | 57 | CDR-H2 | D | E | E | D | D |
| 57 | 58 | CDR-H2 | T | T | T | T | T |
| 58 | 59 | CDR-H2 | K | I | I | K | K |
| 59 | 60 | CDR-H2 | Y | Y | Y | Y | Y |
| 60 | 61 | CDR-H2 | A | A | A | A | A |
| 61 | 62 | CDR-H2 | S | E | E | S | S |
| 62 | 63 | CDR-H2 | K | K | K | K | K |
| 63 | 64 | CDR-H2 | F | F | F | F | F |
| 64 | 65 | CDR-H2 | Q | Q | Q | Q | Q |
| 65 | 66 | CDR-H2 | G | G | G | G | G |
| 66 | 67 | Fr3 | K | R | R | R | R |
| 67 | 68 | Fr3 | A | V | V | A | A |
| 68 | 69 | Fr3 | T | T | T | T | T |
| 69 | 70 | Fr3 | M | I | I | M | M |
| 70 | 71 | Fr3 | T | T | T | T | T |
| 71 | 72 | Fr3 | A | A | A | A | A |
| 72 | 73 | Fr3 | D | D | D | D | D |
| 73 | 74 | Fr3 | T | T | T | T | T |
| 74 | 75 | Fr3 | S | S | S | S | S |
| 75 | 76 | Fr3 | S | T | T | T | T |
| 76 | 77 | Fr3 | N | D | D | D | D |
| 77 | 78 | Fr3 | T | T | T | T | T |
| 78 | 79 | Fr3 | A | A | A | A | A |
| 79 | 80 | Fr3 | Y | Y | Y | Y | Y |
| 80 | 81 | Fr3 | L | M | M | M | M |
| 81 | 82 | Fr3 | Q | E | E | E | E |
| 82 | 83 | Fr3 | L | L | L | L | L |
| 82A | 84 | Fr3 | S | S | S | S | S |
| 82B | 85 | Fr3 | S | S | S | S | S |
| 82C | 86 | Fr3 | L | L | L | L | L |
| 83 | 87 | Fr3 | T | R | R | R | R |
| 84 | 88 | Fr3 | S | S | S | S | S |
| 85 | 89 | Fr3 | E | E | E | E | E |
| 86 | 90 | Fr3 | D | D | D | D | D |
| 87 | 91 | Fr3 | T | T | T | T | T |
| 88 | 92 | Fr3 | A | A | A | A | A |
| 89 | 93 | Fr3 | V | V | V | V | V |
| 90 | 94 | Fr3 | Y | Y | Y | Y | Y |
| 91 | 95 | Fr3 | Y | Y | Y | Y | Y |
| 92 | 96 | Fr3 | C | C | C | C | C |
| 93 | 97 | Fr3 | T | A | A | T | T |
| 94 | 98 | Fr3 | T | T | R | T | T |
| 95 | 99 | CDR-H3 | S | | I | S | S |
| 96 | | CDR-H3 | — | — | P | — | — |
| 97 | | CDR-H3 | — | — | L | — | — |
| 98 | | CDR-H3 | — | — | F | — | — |
| 99 | | CDR-H3 | — | — | G | — | — |
| 100 | | CDR-H3 | — | — | R | — | — |
| 100A | | CDR-H3 | — | — | — | — | — |
| 100B | | CDR-H3 | — | — | — | — | — |
| 100C | | CDR-H3 | — | — | — | — | — |
| 100D | | CDR-H3 | — | — | — | — | — |
| 100E | | CDR-H3 | — | — | — | — | — |
| 100F | | CDR-H3 | — | — | — | — | — |
| 100G | | CDR-H3 | — | — | — | — | — |
| 100H | | CDR-H3 | — | — | — | — | — |
| 100I | | CDR-H3 | — | — | — | — | — |
| 100J | | CDR-H3 | — | — | — | — | — |
| 100K | | CDR-H3 | — | — | — | — | — |
| 101 | 100 | CDR-H3 | N | Q | D | N | N |
| 102 | 101 | CDR-H3 | G | H | H | G | G |
| 103 | 102 | Fr4 | W | W | W | W | W |
| 104 | 103 | Fr4 | G | G | G | G | G |
| 105 | 104 | Fr4 | Q | Q | Q | Q | Q |
| 106 | 105 | Fr4 | G | G | G | G | G |
| 107 | 106 | Fr4 | T | T | T | T | T |
| 108 | 107 | Fr4 | L | L | T | T | L |
| 109 | 108 | Fr4 | V | V | V | V | V |
| 110 | 109 | Fr4 | T | T | T | T | T |
| 111 | 110 | Fr4 | V | V | V | V | V |
| 112 | 111 | Fr4 | S | S | S | S | S |
| 113 | 112 | Fr4 | A | S | R | R | S |

TABLE 40

| | | | | IGKV2-29*02 & | QDO16713- | | |
|---|---|---|---|---|---|---|---|
| | | | Mouse 17C12 VL | IGKJ4*01 | VL_huFrwk | Hu17C12VLv1 | Hu17C12VLv2 |
| Kabat | Linear | | (SEQ ID | (SEQ ID | (SEQ ID | (SEQ ID | (SEQ ID |
| residue # | residue # | FR or CDR | NO: )228) | NO: 239) | NO: 238) | NO: 234) | NO: 235) |
| 1 | 1 | Fr1 | D | D | D | D | D |
| 2 | 2 | Fr1 | V | I | I | V | V |
| 3 | 3 | Fr1 | V | V | V | V | V |
| 4 | 4 | Fr1 | M | M | M | M | M |
| 5 | 5 | Fr1 | T | T | T | T | T |
| 6 | 6 | Fr1 | Q | Q | Q | Q | Q |
| 7 | 7 | Fr1 | T | T | T | T | T |
| 8 | 8 | Fr1 | P | P | P | P | P |
| 9 | 9 | Fr1 | L | L | L | L | L |
| 10 | 10 | Fr1 | T | S | S | S | S |
| 11 | 11 | Fr1 | L | L | L | L | L |
| 12 | 12 | Fr1 | S | S | S | S | S |
| 13 | 13 | Fr1 | V | V | V | V | V |
| 14 | 14 | Fr1 | T | T | T | T | T |
| 15 | 15 | Fr1 | I | P | P | P | P |
| 16 | 16 | Fr1 | G | G | G | G | G |
| 17 | 17 | Fr1 | Q | Q | Q | Q | Q |
| 18 | 18 | Fr1 | P | P | P | P | P |
| 19 | 19 | Fr1 | A | A | A | A | A |
| 20 | 20 | Fr1 | S | S | S | S | S |
| 21 | 21 | Fr1 | I | I | I | I | I |
| 22 | 22 | Fr1 | S | S | S | S | S |
| 23 | 23 | Fr1 | C | C | C | C | C |
| 24 | 24 | CDR-L1 | T | K | K | T | T |
| 25 | 25 | CDR-L1 | S | S | S | S | S |
| 26 | 26 | CDR-L1 | S | S | S | S | S |
| 27 | 27 | CDR-L1 | Q | Q | Q | Q | Q |
| 27A | 28 | CDR-L1 | S | S | S | S | S |
| 278 | 29 | CDR-L1 | L | L | L | L | L |
| 27C | 30 | CDR-L1 | L | L | L | L | L |
| 27D | 31 | CDR-L1 | H | H | H | H | H |
| 27E | 32 | CDR-L1 | S | S | S | S | S |
| 27F | | CDR-L1 | — | — | — | — | — |
| 28 | 33 | CDR-L1 | N | D | D | N | N |
| 29 | 34 | CDR-L1 | R | G | G | R | R |
| 30 | 35 | CDR-L1 | K | K | K | K | K |
| 31 | 36 | CDR-L1 | T | T | T | T | T |
| 32 | 37 | CDR-L1 | Y | Y | Y | Y | Y |
| 33 | 38 | CDR-L1 | L | L | L | L | L |
| 34 | 39 | CDR-L1 | H | Y | Y | H | H |
| 35 | 40 | Fr2 | W | W | W | W | W |
| 36 | 41 | Fr2 | L | Y | Y | L | L |
| 37 | 42 | Fr2 | L | L | L | L | L |
| 38 | 43 | Fr2 | Q | Q | Q | Q | Q |
| 39 | 44 | Fr2 | R | K | K | K | K |
| 40 | 45 | Fr2 | P | P | P | P | P |
| 41 | 46 | Fr2 | G | G | G | G | G |
| 42 | 47 | Fr2 | Q | Q | Q | Q | Q |
| 43 | 48 | Fr2 | S | S | P | P | S |
| 44 | 49 | Fr2 | P | P | P | P | P |
| 45 | 50 | Fr2 | K | Q | Q | Q | Q |
| 46 | 51 | Fr2 | L | L | L | L | L |
| 47 | 52 | Fr2 | L | L | L | L | L |
| 48 | 53 | Fr2 | I | I | I | I | I |
| 49 | 54 | Fr2 | Y | Y | Y | Y | Y |
| 50 | 55 | CDR-L2 | L | E | E | L | L |
| 51 | 56 | CDR-L2 | V | V | V | V | V |
| 52 | 57 | CDR-L2 | S | S | S | S | S |
| 53 | 58 | CDR-L2 | K | S | N | K | K |
| 54 | 59 | CDR-L2 | L | R | R | L | L |
| 55 | 60 | CDR-L2 | E | F | F | E | E |
| 56 | 61 | CDR-L2 | S | S | S | S | S |
| 57 | 62 | Fr3 | G | G | G | G | G |
| 58 | 63 | Fr3 | V | V | V | V | V |
| 59 | 64 | Fr3 | P | P | P | P | P |
| 60 | 65 | Fr3 | D | D | D | D | D |
| 61 | 66 | Fr3 | R | R | R | R | R |
| 62 | 67 | Fr3 | F | F | F | F | F |
| 63 | 68 | Fr3 | S | S | S | S | S |
| 64 | 69 | Fr3 | G | G | G | G | G |
| 65 | 70 | Fr3 | S | S | S | S | S |
| 66 | 71 | Fr3 | G | G | G | G | G |
| 67 | 72 | Fr3 | S | S | S | S | S |
| 68 | 73 | Fr3 | G | G | G | G | G |
| 69 | 74 | Fr3 | T | T | T | T | T |
| 70 | 75 | Fr3 | D | D | D | D | D |
| 71 | 76 | Fr3 | F | F | F | F | F |
| 72 | 77 | Fr3 | T | T | T | T | T |
| 73 | 78 | Fr3 | L | L | L | L | L |
| 74 | 79 | Fr3 | K | K | K | K | K |
| 75 | 80 | Fr3 | I | I | I | I | I |
| 76 | 81 | Fr3 | S | S | S | S | S |
| 77 | 82 | Fr3 | R | R | R | R | R |
| 78 | 83 | Fr3 | V | V | V | V | V |
| 79 | 84 | Fr3 | E | E | E | E | E |
| 80 | 85 | Fr3 | A | A | A | A | A |
| 81 | 86 | Fr3 | E | E | E | E | E |
| 82 | 87 | Fr3 | D | D | D | D | D |
| 83 | 88 | Fr3 | L | V | V | V | V |
| 84 | 89 | Fr3 | G | G | G | G | G |
| 85 | 90 | Fr3 | V | V | V | V | V |
| 86 | 91 | Fr3 | Y | Y | Y | Y | Y |
| 87 | 92 | Fr3 | Y | Y | Y | Y | Y |
| 88 | 93 | Fr3 | C | C | C | C | C |
| 89 | 94 | CDR-L3 | L | M | M | L | L |
| 90 | 95 | CDR-L3 | Q | Q | Q | Q | Q |
| 91 | 96 | CDR-L3 | T | G | S | T | T |
| 92 | 97 | CDR-L3 | T | I | I | T | T |
| 93 | 98 | CDR-L3 | H | H | Q | H | H |
| 94 | 99 | CDR-L3 | F | L | L | F | F |
| 95 | 100 | CDR-L3 | P | P | P | P | P |
| 95A | | CDR-L3 | — | — | — | — | — |
| 95B | | CDR-L3 | — | — | — | — | — |
| 95C | | CDR-L3 | — | — | — | — | — |
| 95D | | CDR-L3 | — | — | — | — | — |
| 95E | | CDR-L3 | — | — | — | — | — |
| 95F | | CDR-L3 | — | — | — | — | — |
| 96 | 101 | CDR-L3 | R | L | P | R | R |
| 97 | 102 | CDR-L3 | T | T | T | T | T |
| 98 | 103 | Fr4 | F | F | F | F | F |
| 99 | 104 | Fr4 | G | G | G | G | G |
| 100 | 105 | Fr4 | G | G | G | G | G |
| 101 | 106 | Fr4 | G | G | G | G | G |
| 102 | 107 | Fr4 | T | T | T | T | T |
| 103 | 108 | Fr4 | K | K | K | K | K |
| 104 | 109 | Fr4 | L | V | V | V | V |
| 105 | 110 | Fr4 | E | E | E | E | E |
| 106 | 111 | Fr4 | I | I | I | I | I |
| 106A | | Fr4 | — | — | — | — | — |
| 107 | 112 | Fr4 | K | K | K | K | K |

TABLE 41
V_{H}, V_{L} Backmutations and Other Mutations for Humanized 17C12

| | | Changes from Acceptor Framework |
|---|---|---|
| | | (or CDR) Residues (based on |
| V_{H} or V_{L} Variant | V_{H} or V_{L} Exon Acceptor Sequence | Kabat/Chothia Composite CDRs) |
| Hu17C12VHv1 | GenBank Acc. # CAC20421-VH_huFrwk | H2, H24, H48, H67, H69, H93, H94 |
| (SEQ ID NO: 232) | (SEQ ID NO: 218) | |
| | IMGT# IGHV1-69-2*01 | |
| | (SEQ ID NO: 33) | |
| Hu17C12VHv2 | GenBank Acc. # CAC20421-VH_huFrwk | H1, H2, H24, H48, H67, H69, H93, H94, |
| (SEQ ID NO: 232) | (SEQ ID NO: 218) | H108, H113 |
| | IMGT# IGHV1-69-2*01 | |
| | (SEQ ID NO: 33) | |
| Hu17C12VLv1 | GenBank Acc. # QDO16713-VL_huFrwk | L2, L36 |
| (SEQ ID NO: 233) | (SEQ ID NO: 238) | |
| Hu17C12VLv2 | GenBank Acc. # QDO16713-VL_huFrwk | L2, L36, L43 |
| (SEQ ID NO: 234) | (SEQ ID NO: 238) | |
| | IGKV2-29*02 & IGKJ4*01 | |
| | (SEQ ID NO: 239) | |

TABLE 42
Kabat Numbering of Framework (or CDR) Residues (based on Kabat/Chothia
Composite CDRs) for Backmutations and Other Mutations in Heavy Chains
of Humanized 17C12 Antibodies

| | CAC20421- | IMGT# | | | |
|---|---|---|---|---|---|
| | VH_huFrwk | IGHV1-69-2*01 | Mouse 17C12 VH | Hu17C12VHv1 | Hu17C12VHv2 |
| | (SEQ ID | (SEQ ID | (SEQ ID | (SEQ ID | (SEQ ID |
| Residue | NO: 218) | NO: 33) | NO: 225) | NO: 232) | NO: 233) |
| H1 | Q | E | K | Q | E |
| H2 | V | V | I | I | I |
| H24 | V | V | A | A | A |
| H48 | M | M | I | I | I |
| H67 | V | V | A | A | A |
| H69 | I | I | M | M | M |
| H93 | A | A | T | T | T |
| H94 | R | T | T | T | T |
| H108 | T | L | L | T | L |
| H113 | R | S | A | R | S |

TABLE 43
Kabat Numbering of Framework Residues (based
on Kabat/Chothia Composite CDRs) for
Backmutations and Other Mutations in
Light Chains of Humanized 17C12
Antibodies

| | | IGKV2- | | | |
|---|---|---|---|---|---|
| | | 29*02 | | | |
| | QD016713- | & | Mouse | | |
| | VL_ | IGKJ4* | I7CI2 | Hu17C12 | Hu17C12 |
| | huFnvk | 01 | VL | VLv1 | VLv2 |
| | (SEQ | (SEQ | (SEQ | (SEQ | (SEQ |
| | ID | ID | ID | ID | ID |
| | NO: | NO: | NO: | NO: | NO: |
| Residue | 238) | 239) | 228) | 234) | 235) |
| L2 | I | I | V | V | V |
| L36 | Y | Y | L | L | L |
| L43 | P | S | S | P | S |

TABLE 44
Percentage Humanness of Heavy and Light
Chains of Humanized 17C12 Antibodies

| V_{H} or V_{L} Variant | % Humanness |
|---|---|
| Hu17C12VHv1 (SEQ ID NO: 232) | 79.6% |
| Hu17C12VHv2 (SEQ ID NO: 233) | 80.6% |
| Hu17C12VLv1 (SEQ ID NO: 234) | 85.0% |
| Hu17C12VLv2 (SEQ ID NO: 235) | 86.0% |

Positions at which canonical, vernier, or interface residues differ between mouse and human acceptor sequences are candidates for substitution. Examples of canonical/CDR interacting residues include Kabat residues H94 in Table 39 and L2 in Table 40. Examples of vernier residues include Kabat residues H2, H24, H48, H67, H69, H93, and H94 in Table 39 and L2 and L36 in Table 40. Examples of interface/packing (VH+VL) residues include Kabat residue H93 in Table 39 and L36 in Table 40.

The rationales for selection of the positions indicated in Table 39 in the heavy chain variable region as candidates for substitution are as follows.

Heavy Chain Variable Regions

hu17C12VHv1
consists of the CDR-H1, H2, and H3 loops of 17C12-VH grafted onto the framework of

CAC20421. Additionally, hu17C12VHv1 also reverts all framework substitutions at positions that are key for defining the Chothia canonical classes, are part of the Vernier zone, or localize to the VH/VL domain interface contribute to structural stability.

V2I: is a backmutation of a Vernier zone residue to preserve Vernier zone.

V24A: is a backmutation of a Chothia canonical structure residue and back-mutation may enhance stability.

M48I: is a backmutation of a Vernier zone residue.

V67A: is a backmutation of a Vernier zone residue and is back mutated to retain CDR conformation.

I69M: is a backmutation of a Vernier zone residue and backmutation is made to keep the CDR packing intact.

A93T: is a backmutation of a VH/VL interface residue, and is backmutated for the integrity of the antibody interface.

R94T: is a backmutation of a Chothia canonical structural residue. R94T is also a germ-line-aligning mutation. Thr is in human germ line gene IGHV1-69-2*01 (SEQ ID NO:33) at this position.

hu17C12VHv2
hu17C12VHv2 also retains all reverted framework substitutions at positions that are key for defining the Chothia canonical classes, are part of the Vernier zone, or localize to the VH/VL domain interface contribute to structural stability. Additionally, hu17C12VHv2 incorporates backmutations or substitution with most frequent residue at a given position and for optimization of deamination, oxidation, N-glycosylation, proteolysis and aggregation. Mutations in hu17C12VHv2 that are not included in hu17C12VHv1 are:

Q1E: is a stability enhancing mutation to mitigate pyroglutamate formation potential to reduce N-term heterogeneity (Liu, supra.).

T108L: is a germ-line aligning mutation. Leu is in human germ line gene IGHV1-69-2*01 (SEQ ID NO:33) at this position.

R113 S: is a frequency-based and germ-line aligning mutation. Arg is rare at this position, and Ser is in human germ line gene IGHV1-69-2*01 (SEQ ID NO:33) at this position and is most frequent residue at this position.

The rationales for selection of the positions indicated in Table 40 in the light chain variable region as candidates for substitution are as follows.

Kappa Light Chain Variable Regions

hu17C12VLv1
consists of the CDR-L1, L2, and L3 loops of 17C12-VL grafted onto the framework of QD016713 VL along with reverting all framework substitutions at positions that are key for defining the Chothia canonical classes, are part of the Vernier zone, or locate to the VH/VL domain interface.

I2V: is a backmutation of a Vernier zone residue and back mutation preserves that CDR packing conformation.

Y36L: is a backmutation of a VH/VL interface and Vernier zone residue, and backmutation is made to keep the interface intact.

hu17C12VLv2
hu17C12VLv2 includes substitutions that contribute to structural stability or increase the humanness of the antibody.

P43S: is a frequency-based and germ line-aligning mutation. Pro is rare at this position and is substituted with Ser which is in human germline gene IGKV2-29*02 & IGKJ4*01 (SEQ ID NO:239) at this position.

The designs based on these human frameworks were:

heavy chain variable regions
>hu17C12VHv1
QIQLVQSGAEVKKPGATVKISCKASAFNIKDDYMNWVQQAP
GKGLEWIGWIDPENGDTKYASKFQGRATMTADTSTDTAYME
LSSLRSEDTAVYYCTTSNGWGQGTTVTVSR
>hu17C12VHv2
EVQLQQSGAELVKPGATVKISCTASGFNIKDDYMNWVKQRP
EQGLEWIGWIDPENGDTEYASKFQGKATMTADTSTNTAYLQ
LSSLTSEDTAVYYCTTSNGWGQGTTVTVSS
kappa light chain variable regions
>hu17C12VLv1
DVVMTQTPLSLSVTPGQPASISCTSSQSLLHSNRKTYLHWL
LQKPGQPPQLLIYLVSKLESGVPDRFSGSGSGTDFTLKISR
VEAEDVGVYYCLQTTHFPRTFGGGTKVEIK
>hu17C12VLv2
DVVMTQTPLSLSVTPGQPASISCTSSQSLLHSNRKTYLHWL
LQKPGQSPQLLIYLVSKLESGVPDRFSGSGSGTDFTLKISR
VEAEDVGVYYCLQTTHFPRTFGGGTKVEIK

Humanized sequences are generated using a two-stage PCR protocol that allows introduction of multiple mutations, deletions, and insertions using QuikChange site-directed mutagenesis [Wang, W. and Malcolm, B. A. (1999) BioTechniques 26:680-682).

### Example 12. Design of Humanized 14H3 Antibodies

The starting point or donor antibody for humanization was the mouse antibody 14H3. The heavy chain variable amino acid sequence of mature m14H3 is provided as SEQ ID NO:240. The light chain variable amino acid sequence of mature m14H3 is provided as SEQ ID NO:244. The heavy chain Kabat/Chothia Composite CDR1, CDR2, and CDR3 amino acid sequences are provided as SEQ ID NOs:241-243, respectively. The light chain Kabat CDR1, CDR2, and CDR3 amino acid sequences are provided as SEQ ID NOs:245-247 respectively. Kabat numbering is used throughout.

The variable kappa (Vk) of 14H3 belongs to mouse Vk subgroup 2which corresponds to human Vk subgroup 2 and the variable heavy (Vh) to mouse Vh subgroup lb which corresponds to human Vh subgroup 2 [Kabat E.A., et al., (1991), Sequences of Proteins of Immunological Interest, Fifth Edition. NIH Publication No. 91-3242]. 16 residue Chothia CDR-L1 is similar to Chothia canonical class 4, 7 residue Chothia CDR-L2 is of Chothia canonical class 1, and 9 residue Chothia CDR-L3 is similar to Chothia canonical class 1 [Martin A C R. (2010) Protein sequence and structure analysis of antibody variable domains. In: Kontermann R and Dübel S (eds). Antibody Engineering. Heidelberg, Germany: Springer International Publishing AG. [Martin, 2010]. 12 residue Chothia CDR-H1 is similar to Chothia canonical class 3, 16 residue Chothia CDR-H2 is similar to Chothia canonical class 1 [Martin, 2010], and 4 residue CDR-H3 has no canonical classes. A search was made over the protein sequences in the PDB database [Deshpande N, et al., (2005) Nucleic Acids Res. 33: D233-7] to find structures, which would provide a rough structural model of 14H3. The crystal structure of an antibody fab pdb code 2VQ1 [De Geus, D. C., et al. Proteins 76: 439; (2009)] was used for both Vh and Vk structure since it had good resolution (2.5A°) and overall sequence similarity to 14H3 Vh and Vk, retaining the same canonical structures for the loops.

The frameworks of 14H3 VH share a high degree of sequence similarity with the corresponding regions of of immunoglobulin heavy chain variable region [Homo sapiens] QDJ57937 cloned by Cassotta et al. [(2009) Cassotta, A., Mikol, V., Bertrand, T.,et al. A single T cell epitope drives the neutralizing anti-idiotypic antibody response to natalizumab in patients with multiple sclerosis Unpublished (direct submission to GenBank (2019)]. The variable domains of 14H3 and QDJ57937 VH also share identical lengths for the CDR-H1, H2 loops. Similarly, the frameworks of 14H3 VL share a high degree of sequence similarity with the corresponding regions of human antibody ABC66914, cloned by Shriner et al. [Vaccine 24 (49-50), 7159-7166 (2006)]. The variable light domain of 14H3 and ABC66914 VL also share identical lengths for the CDR-L1, L2 and L3 loops. Accordingly, the framework regions of QDJ57937 VH and ABC66914 VL were chosen as the acceptor sequences for the CDRs of 14H3. A model of the 14H3 CDRs grafted onto the respective human frameworks for VH and VL was built and used as a guidance for further backmutations.

Heavy and light chain variant sequences resulting from antibody humanization process were further aligned to human germ line sequences using IMGT Domain GapAlign tool to assess the humanness of the heavy and light chain as outlined by WHO INN committee guidelines. (WHO-INN: International nonproprietary names (INN) for biological and biotechnological substances (a review) (Internet) 2014. Available from: http://www.who.int/medicines/services/inn/BioRev2014.pdf) Residues were changed to align with corresponding human germ line sequence, where possible, to enhance humanness and to reduce potential immunogenicity. For humanized VLv1 and VLv2 variants, mutations were introduced to render the sequences more similar to human germ line gene IGKV2-28*01 & IGKJ2*01 (SEQ ID NO:37). For humanized VHv1 and VHv2 variants, mutations were introduced to render the sequences more similar to human germ line gene IGHV1-70*04 & IGHJ4*01 (SEQ ID NO:254)

Additional versions of hu14H3-VH and hu14H3-VL were designed to enable assessment of various framework residues for their contributions to antigen binding, thermostability, and immunogenicity, and for optimization of deamination, oxidation, N-glycosylation, proteolysis and aggregation. The positions considered for mutation include those that:
define the canonical CDR conformations (summarized in Martin, A. C. R. (2010) Protein sequence and structure analysis of antibody variable domains. In: Kontermann R and Dübel S (eds). Antibody Engineering. Heidelberg, Germany: Springer International Publishing AG.),
are within the Vernier zone (Foote J and Winter G. (1992) Antibody framework residues affecting the conformation of the hypervariable loops. J Mol Biol. 224(2):487-99.),
localize to the VH/VL domain interface (summarized in Léger O J P and Saldanha J. (2000) Preparation of recombinant antibodies from immune rodent spleens and the design of their humanisation by CDR grafting. In: Shepherd P and Dean C (eds). Monoclonal Antibodies: a Practical Approach. Oxford, UK: Oxford University Press.),
are susceptible to post-translational modifications, such as glycosylation or pyroglutamination,
are occupied by residues that are predicted to clash with CDRs, according to the model of 14H3 CDRs grafted onto VH and VL frameworks, or
are occupied by residues that are rare among sequenced human antibodies, where either the parental mouse 14H3 residue or some other residue is much more prevalent within human antibody repertoire.

Alignments of the murine 14H3 and various humanized antibodies are shown for the light chain variable regions (Table 46 and FIG. 14), and heavy chain variable regions (Table 45 and FIG. 13).

2 humanized heavy chain variable region variants and 2 humanized light chain variable region variants were constructed containing different permutations of substitutions: hu14H3VHv1 or hu14H3VHv2, (SEQ ID NOs:248-249, respectively); and hu14H3VLv1 or hu14H3VLv2, (SEQ ID NOs:250-251, respectively) (Tables 45 and 46). The exemplary humanized Vk and Vh designs, with backmutations and other mutations based on selected human frameworks, are shown in Tables 45 and 46, respectively. The bolded areas in Tables 45 and 46 indicate the CDRs as defined by Kabat/Chothia Composite. A “-” in the columns in Tables 45 and 46indicates no residue at the indicated position. SEQ ID NOs:248-249, and SEQ ID NOs:250-251 contain backmutations and other mutations as shown in Table 47. The amino acids at positions in hu14H3VHv1 and hu14H3VHv2 are listed in Table 48 The amino acids at positions in hu14H3VLv1 and hu14H3VLv2 are listed in Table 49.

The percentage humanness for humanized VH chains hu14H3VHv1 and hu14H3VHv2 (SEQ ID NOs:248-249, respectively) with respect to the most similar human germline gene IGHV1-70*04 & IGHJ4*01 (SEQ ID NO:254), and for humanized VL chains hu14H3VLv1 and hu14H3VLv2 (SEQ ID NOs:250-251, respectively) with respect to the most similar human germline gene IGKV2-28*01 & IGKJ2*01 (SEQ ID NO:37), is shown in Table 50.

TABLE 45

| | | | | IGHV2- | | | |
|---|---|---|---|---|---|---|---|
| | | | | 70*04 | | | |
| | | | Mouse | & | QDJ57 | | |
| | | | 14H3 | IGHJ4* | 937VH | Hu4H3 | Hu14H3 |
| | | | VH | 01 | hFrwk | VHv1 | VHv2 |
| Kabat | Linear | | (SEQ | (SEQ | (SEQ | (SEQ | (SEQ |
| resi- | resi- | FR | ID | ID | ID | ID | ID |
| due | due | or | NO: | NO: | NO: | NO: | NO: |
| # | # | CDR | 240) | 254) | x253 | 248) | 249) |
| 1 | 1 | Fr1 | Q | Q | Q | Q | Q |
| 2 | 2 | Fr1 | V | V | V | V | V |
| 3 | 3 | Fr1 | T | T | T | T | T |
| 4 | 4 | Fr1 | L | L | L | L | L |
| 5 | 5 | Fr1 | K | K | K | K | K |
| 6 | 6 | Fr1 | E | E | E | E | E |
| 7 | 7 | Fr1 | S | S | S | S | S |
| 8 | 8 | Fr1 | G | G | G | G | G |
| 9 | 9 | Fr1 | P | P | P | P | P |
| 10 | 10 | Fr1 | G | A | A | A | A |
| 11 | 11 | Fr1 | 1 | L | L | L | L |
| 12 | 12 | Fr1 | L | V | V | V | V |
| 13 | 13 | Fr1 | Q | K | K | K | K |
| 14 | 14 | Fr1 | P | P | P | P | P |
| 15 | 15 | Fr1 | S | T | T | T | T |
| 16 | 16 | Fr1 | Q | Q | Q | Q | Q |
| 17 | 17 | Fr1 | T | T | T | T | T |
| 18 | 18 | Fr1 | L | L | L | L | L |
| 19 | 19 | Fr1 | S | T | T | T | T |
| 20 | 20 | Fr1 | L | L | L | L | L |
| 21 | 21 | Fr1 | T | T | T | T | T |
| 22 | 22 | Fr1 | C | C | C | C | C |
| 23 | 23 | Fr1 | S | T | T | T | T |
| 24 | 24 | Fr1 | F | F | F | F | F |
| 25 | 25 | Fr1 | S | S | S | S | S |
| 26 | 26 | CDR- | G | G | G | G | G |
| | | H1 | | | | | |
| 27 | 27 | CDR- | F | F | F | F | F |
| | | H1 | | | | | |
| 28 | 28 | CDR- | S | S | S | S | S |
| | | H1 | | | | | |
| 29 | 29 | CDR- | L | L | L | L | L |
| | | H1 | | | | | |
| 30 | 30 | CDR- | S | S | S | S | S |
| | | H1 | | | | | |
| 31 | 31 | CDR- | T | T | T | T | T |
| | | H1 | | | | | |
| 32 | 32 | CDR- | Y | S | S | Y | Y |
| | | H1 | | | | | |
| 33 | 33 | CDR- | G | G | G | G | G |
| | | H1 | | | | | |
| 34 | 34 | CDR- | M | M | M | M | M |
| | | H1 | | | | | |
| 35 | 35 | CDR- | G | R | R | G | G |
| | | H1 | | | | | |
| 35A | 36 | CDR- | V | V | V | V | V |
| | | H1 | | | | | |
| 35B | 37 | CDR- | G | S | S | S | S |
| | | H1 | | | | | |
| 36 | 38 | Fr2 | W | W | W | W | W |
| 37 | 39 | Fr2 | I | I | I | I | I |
| 38 | 40 | Fr2 | R | R | R | R | R |
| 39 | 41 | Fr2 | Q | Q | Q | Q | Q |
| 40 | 42 | Fr2 | P | P | P | P | P |
| 41 | 43 | Fr2 | S | P | P | P | P |
| 42 | 44 | Fr2 | G | G | G | G | G |
| 43 | 45 | Fr2 | K | K | K | K | K |
| 44 | 46 | Fr2 | G | A | A | A | A |
| 45 | 47 | Fr2 | L | L | L | L | L |
| 46 | 48 | Fr2 | E | E | E | E | E |
| 47 | 49 | Fr2 | W | W | W | W | W |
| 48 | 50 | Fr2 | L | L | L | L | L |
| 49 | 51 | Fr2 | A | A | A | A | A |
| 50 | 52 | CDR-H2 | N | R | R | N | N |
| 51 | 53 | CDR-H2 | I | I | I | I | I |
| 52 | 54 | CDR-H2 | W | D | D | W | W |
| 52A | | CDR-H2 | — | — | — | — | — |
| 52B | | CDR-H2 | — | — | — | — | — |
| 52C | | CDR-H2 | — | — | — | — | — |
| 53 | 55 | CDR-H2 | W | W | W | W | W |
| 54 | 56 | CDR-H2 | D | D | D | D | D |
| 55 | 57 | CDR-H2 | D | D | D | D | D |
| 56 | 58 | CDR-H2 | I | D | D | I | I |
| 57 | 59 | CDR-H2 | K | K | K | K | K |
| 58 | 60 | CDR-H2 | Y | F | F | Y | Y |
| 59 | 61 | CDR-H2 | Y | Y | Y | Y | Y |
| 60 | 62 | CDR-H2 | N | S | S | N | N |
| 61 | 63 | CDR-H2 | A | T | T | A | A |
| 62 | 64 | CDR-H2 | A | S | S | A | A |
| 63 | 65 | CDR-H2 | L | L | L | L | L |
| 64 | 66 | CDR-H2 | K | K | K | K | K |
| 65 | 67 | CDR-H2 | S | T | T | S | S |
| 66 | 68 | Fr3 | R | R | R | R | R |
| 67 | 69 | Fr3 | L | L | L | L | L |
| 68 | 70 | Fr3 | T | T | T | T | T |
| 69 | 71 | Fr3 | I | I | I | I | I |
| 70 | 72 | Fr3 | S | S | S | S | S |
| 71 | 73 | Fr3 | K | K | K | K | K |
| 72 | 74 | Fr3 | D | D | D | D | D |
| 73 | 75 | Fr3 | T | T | T | T | T |
| 74 | 76 | Fr3 | S | S | S | S | S |
| 75 | 77 | Fr3 | K | K | K | K | K |
| 76 | 78 | Fr3 | N | N | N | N | N |
| 77 | 79 | Fr3 | Q | Q | Q | Q | Q |
| 78 | 80 | Fr3 | V | V | V | V | V |
| 79 | 81 | Fr3 | F | V | V | V | V |
| 80 | 82 | Fr3 | L | L | L | L | L |
| 81 | 83 | Fr3 | K | T | T | T | T |
| 82 | 84 | Fr3 | I | M | M | M | M |
| 82A | 85 | Fr3 | A | T | T | T | T |
| 82B | 86 | Fr3 | S | N | N | N | N |
| 82C | 87 | Fr3 | V | M | M | M | M |
| 83 | 88 | Fr3 | D | D | D | D | D |
| 84 | 89 | Fr3 | T | P | P | P | P |
| 85 | 90 | Fr3 | A | V | V | V | V |
| 86 | 91 | Fr3 | D | D | D | D | D |
| 87 | 92 | Fr3 | T | T | T | T | T |
| 88 | 93 | Fr3 | A | A | A | A | A |
| 89 | 94 | Fr3 | T | T | T | T | T |
| 90 | 95 | Fr3 | Y | Y | Y | Y | Y |
| 91 | 96 | Fr3 | Y | Y | Y | Y | Y |
| 92 | 97 | Fr3 | C | C | C | C | C |
| 93 | 98 | Fr3 | A | A | A | A | A |
| 94 | 99 | Fr3 | R | R | R | R | R |
| 95 | 100 | CDR-H3 | N | N | L | N | N |
| 96 | 101 | CDR-H3 | V | V | A | V | V |
| 97 | | CDR-H3 | — | — | V | — | — |
| 98 | | CDR-H3 | — | — | A | — | — |
| 99 | | CDR-H3 | — | — | D | — | — |
| 100 | | CDR-H3 | — | — | A | — | — |
| 100A | | CDR-H3 | — | — | F | — | — |
| 100B | | CDR-H3 | — | — | — | — | — |
| 100C | | CDR-H3 | — | — | — | — | — |
| 100D | | CDR-H3 | — | — | — | — | — |
| 100E | | CDR-H3 | — | — | — | — | — |
| 100F | | CDR-H3 | — | — | — | — | — |
| 100G | | CDR-H3 | — | — | — | — | — |
| 100H | | CDR-H3 | — | — | — | — | — |
| 100I | | CDR-H3 | — | — | — | — | — |
| 100J | | CDR-H3 | — | — | — | — | — |
| 100K | | CDR-H3 | — | — | — | — | — |
| 101 | 102 | CDR-H3 | D | D | D | D | D |
| 102 | 103 | CDR-H3 | Y | Y | I | Y | Y |
| 103 | 104 | Fr4 | W | W | W | W | W |
| 104 | 105 | Fr4 | G | G | G | G | G |
| 105 | 106 | Fr4 | Q | Q | Q | Q | Q |
| 106 | 107 | Fr4 | G | G | G | G | G |
| 107 | 108 | Fr4 | T | T | T | T | T |
| 108 | 109 | Fr4 | T | L | M | M | L |
| 109 | 110 | Fr4 | L | V | V | V | V |
| 110 | 111 | Fr4 | T | T | T | T | T |
| 111 | 112 | Fr4 | V | V | V | V | V |
| 112 | 113 | Fr4 | S | S | S | S | S |
| 113 | 114 | Fr4 | S | S | L | L | S |

TABLE 46

| | | | | 28*01 | ABC669 | | |
|---|---|---|---|---|---|---|---|
| | | | Mouse | & | 14VL_ | Hu14H | Hu14 |
| | | | 14H3 | IGKJ2* | hFwrk | 3VLv1 | H3VLv2 |
| Kabat | Linear | | VL | 01 | (SEQ | (SEQ | (SEQ |
| resi— | resi— | | (SEQ | (SEQ | ID | ID | ID |
| due | due | FR or | ID NO: | ID NO: | NO: | NO: | NO: |
| # | # | CDR | 244) | 37) | 256) | 250) | 251) |
| 1 | 1 | Fr1 | D | D | D | D | D |
| 2 | 2 | Fr1 | V | I | I | V | V |
| 3 | 3 | Fr1 | V | V | V | V | V |
| 4 | 4 | Fr1 | M | M | M | M | M |
| 5 | 5 | Fr1 | T | T | T | T | T |
| 6 | 6 | Fr1 | Q | Q | Q | Q | Q |
| 7 | 7 | Fr1 | T | S | T | T | S |
| 8 | 8 | Fr1 | P | P | P | P | P |
| 9 | 9 | Fr1 | L | L | L | L | L |
| 10 | 10 | Fr1 | S | S | S | S | S |
| 11 | 11 | Fr1 | L | L | L | L | L |
| 12 | 12 | Fr1 | P | P | P | P | P |
| 13 | 13 | Fr1 | V | V | V | V | V |
| 14 | 14 | Fr1 | S | T | T | T | T |
| 15 | 15 | Fr1 | L | P | P | P | P |
| 16 | 16 | Fr1 | G | G | G | G | G |
| 17 | 17 | Fr1 | D | E | E | E | E |
| 18 | 18 | Fr1 | Q | P | P | P | P |
| 19 | 19 | Fr1 | A | A | A | A | A |
| 20 | 20 | Fr1 | S | S | S | S | S |
| 21 | 21 | Fr1 | I | I | I | I | I |
| 22 | 22 | Fr1 | S | S | S | S | S |
| 23 | 23 | Fr1 | C | C | C | C | C |
| 24 | 24 | CDR-L1 | R | R | R | R | R |
| 25 | 25 | CDR-L1 | S | S | S | S | S |
| 26 | 26 | CDR-L1 | S | S | S | S | S |
| 27 | 27 | CDR-L1 | Q | Q | Q | Q | Q |
| 27A | 28 | CDR-L1 | S | S | S | S | S |
| 27B | 29 | CDR-L1 | L | L | L | L | L |
| 27C | 30 | CDR-L1 | V | L | L | V | V |
| 27D | 31 | CDR-L1 | H | H | H | H | H |
| 27E | 32 | CDR-L1 | S | S | S | S | S |
| 27F | | CDR-L1 | — | — | — | — | — |
| 28 | 33 | CDR-L1 | N | N | N | N | N |
| 29 | 34 | CDR-L1 | G | G | G | G | G |
| 30 | 35 | CDR-L1 | N | Y | Y | N | N |
| 31 | 36 | CDR-L1 | T | N | N | T | T |
| 32 | 37 | CDR-L1 | F | Y | Y | F | F |
| 33 | 38 | CDR-L1 | L | L | L | L | L |
| 34 | 39 | CDR-L1 | H | D | D | H | H |
| 35 | 40 | Fr2 | W | W | W | W | W |
| 36 | 41 | Fr2 | Y | Y | Y | Y | Y |
| 37 | 42 | Fr2 | L | L | L | L | Q |
| 38 | 43 | Fr2 | Q | Q | Q | Q | Q |
| 39 | 44 | Fr2 | K | K | K | K | K |
| 40 | 45 | Fr2 | P | P | P | P | P |
| 41 | 46 | Fr2 | G | G | G | G | G |
| 42 | 47 | Fr2 | Q | Q | Q | Q | Q |
| 43 | 48 | Fr2 | S | S | S | S | S |
| 44 | 49 | Fr2 | P | P | P | P | P |
| 45 | 50 | Fr2 | K | Q | Q | Q | Q |
| 46 | 51 | Fr2 | L | L | L | L | L |
| 47 | 52 | Fr2 | L | L | L | L | L |
| 48 | 53 | Fr2 | I | I | I | I | I |
| 49 | 54 | Fr2 | Y | Y | Y | Y | Y |
| 50 | 55 | CDR-L2 | K | L | L | K | K |
| 51 | 56 | CDR-L2 | V | G | G | V | V |
| 52 | 57 | CDR-L2 | S | S | S | S | S |
| 53 | 58 | CDR-L2 | N | N | N | N | N |
| 54 | 59 | CDR-L2 | R | R | R | R | R |
| 55 | 60 | CDR-L2 | F | A | A | F | F |
| 56 | 61 | CDR-L2 | S | S | S | S | S |
| 57 | 62 | Fr3 | G | G | G | G | G |
| 58 | 63 | Fr3 | V | V | V | V | V |
| 59 | 64 | Fr3 | P | P | P | P | P |
| 60 | 65 | Fr3 | D | D | D | D | D |
| 61 | 66 | Fr3 | R | R | R | R | R |
| 62 | 67 | Fr3 | F | F | F | F | F |
| 63 | 68 | Fr3 | S | S | S | S | S |
| 64 | 69 | Fr3 | G | G | G | G | G |
| 65 | 70 | Fr3 | S | S | S | S | S |
| 66 | 71 | Fr3 | G | G | G | G | G |
| 67 | 72 | Fr3 | S | S | S | S | S |
| 68 | 73 | Fr3 | G | G | G | G | G |
| 69 | 74 | Fr3 | T | T | T | T | T |
| 70 | 75 | Fr3 | D | D | D | D | D |
| 71 | 76 | Fr3 | F | F | F | F | F |
| 72 | 77 | Fr3 | T | T | T | T | T |
| 73 | 78 | Fr3 | L | L | L | L | L |
| 74 | 79 | Fr3 | K | K | K | K | K |
| 75 | 80 | Fr3 | I | I | I | I | I |
| 76 | 81 | Fr3 | S | S | S | S | S |
| 77 | 82 | Fr3 | R | R | R | R | R |
| 78 | 83 | Fr3 | V | V | V | V | V |
| 79 | 84 | Fr3 | E | E | E | E | E |
| 80 | 85 | Fr3 | A | A | A | A | A |
| 81 | 86 | Fr3 | E | E | E | E | E |
| 82 | 87 | Fr3 | D | D | D | D | D |
| 83 | 88 | Fr3 | L | V | V | V | V |
| 84 | 89 | Fr3 | G | G | G | G | G |
| 85 | 90 | Fr3 | V | V | V | V | V |
| 86 | 91 | Fr3 | Y | Y | Y | Y | Y |
| 87 | 92 | Fr3 | F | Y | Y | F | F |
| 88 | 93 | Fr3 | C | C | C | C | C |
| 89 | 94 | CDR-L3 | S | M | M | S | S |
| 90 | 95 | CDR-L3 | Q | Q | Q | Q | Q |
| 91 | 96 | CDR-L3 | S | A | A | S | S |
| 92 | 97 | CDR-L3 | T | L | L | T | T |
| 93 | 98 | CDR-L3 | L | Q | Q | L | L |
| 94 | 99 | CDR-L3 | V | T | T | V | V |
| 95 | 100 | CDR-L3 | P | P | P | P | P |
| 95A | | CDR-L3 | — | — | L | — | — |
| 95B | | CDR-L3 | — | — | T | — | — |
| 95C | | CDR-L3 | — | — | — | — | — |
| 95D | | CDR-L3 | — | — | — | — | — |
| 95E | | CDR-L3 | — | — | — | — | — |
| 95F | | CDR-L3 | — | — | — | — | — |
| 96 | 101 | CDR-L3 | W | Y | L | W | W |
| 97 | 102 | CDR-L3 | T | T | T | T | T |
| 98 | 103 | Fr4 | F | F | F | F | F |
| 99 | 104 | Fr4 | G | G | G | G | G |
| 100 | 105 | Fr4 | G | Q | G | G | Q |
| 101 | 106 | Fr4 | G | G | G | G | G |
| 102 | 107 | Fr4 | T | T | T | T | T |
| 103 | 108 | Fr4 | K | K | K | K | K |
| 104 | 109 | Fr4 | L | L | V | V | L |
| 105 | 110 | Fr4 | E | E | E | E | E |
| 106 | 111 | Fr4 | I | I | I | I | I |
| 106A | | Fr4 | — | — | — | — | — |
| 107 | 112 | Fr4 | K | K | K | K | K |

TABLE 47
V_{H}, V_{L} Backmutations and Other Mutations for Humanized 14H3

| | | Changes from Acceptor Framework |
|---|---|---|
| | | (or CDR) Residues (based on |
| V_{H} or V_{L} Variant | V_{H} or V_{L} Exon Acceptor Sequence | Kabat/Chothia Composite CDRs) |
| Hu14H3VHv1 | GenBank Acc. # QDJ57937VH hFrwk | H35B |
| (SEQ ID NO: 248 | (SEQ ID NO: 253) | |
| | IGHV2-70*04 & IGHJ4*01 | |
| | (SEQ ID NO: 254) | |
| Hu14H3VHv2 | GenBank Acc. # QDJ57937VH hFrwk | H35B, H108, H113 |
| (SEQ ID NO: 249) | (SEQ ID NO: 253) | |
| | IGHV2-70*04 & IGHJ4*01 | |
| | (SEQ ID NO: 254) | |
| Hu14H3VLv1 | GenBank Acc. # ABC66914VL_hFwrk | L2, L87 |
| (SEQ ID NO: 250) | (SEQ ID NO: 256) | |
| Hu14H3VLv2 | GenBank Acc. # ABC66914VL_hFwrk | L2, L7, L37, L87, L100, L104 |
| (SEQ ID NO: 251) | (SEQ ID NO: 256) | |
| | IGKV2-28*01 & IGKJ2*01 | |
| | (SEQ ID NO: 37) | |

TABLE 48
Kabat Numbering of Framework Composite CDRs)
for Backmutations14H3 Antibodies (or CDR)
Residues (based on Kabat/Chothia and Other
Mutations in Heavy Chains of Humanized

| | | IGHV2- | | | |
|---|---|---|---|---|---|
| | | 70*04 | | | |
| | QDJ579 | & | Mouse | | |
| | 37VH | IGHJ4* | 14H3 | Hu14H3 | Hu14H3 |
| | hFnvk | 01 | VH | VHv1 | VHv2 |
| | (SEQ | (SEQ | (SEQ | (SEQ | (SEQ |
| | ID | ID | ID | ID | ID |
| Resi- | NO: | NO: | NO: | NO: | NO: |
| due | 253) | 254) | 240) | 248) | 249) |
| H35B | S | S | G | S | S |
| H108 | M | L | T | M | L |
| H113 | L | S | S | L | S |

TABLE 49
Kabat Numbering of Framework Residues
(based on Kabat/Chothia Composite CDRs)
for Backmutations and Otber Mutations
in Light Chains of Humanized 14H3
Antibodies

| | | IGKV2- | | | |
|---|---|---|---|---|---|
| | ABC66 | 28*01 | Mouse | | |
| | 914VL | & IGKJ2* | 14H3 | Hu14H3 | Hu14H3 |
| | hFwrk | 01 | VL | VLv1 | VLv2 |
| | (SEQ | (SEQ | (SEQ | (SEQ | (SEQ |
| | ID | ID | ID | ID | ID |
| Resi- | NO: | NO: | NO: | NO: | NO: |
| due | 256) | 37) | x244) | 250) | 251) |
| L2 | I | I | V | V | V |
| L7 | T | S | T | T | s |
| L37 | L | L | L | L | Q |
| L87 | Y | Y | F | F | F |
| L100 | G | Q | G | G | Q |
| L104 | V | L | L | V | L |

TABLE 50
Percentage Humanness of Heavy and Light
Chains of Humanized 14H3 Antibodies

| V_{H} or V_{L} Variant | % Humanness |
|---|---|
| Hu14H3VHv1 (SEQ ID NO: 248) | 89.9% |
| Hu14H3VHv2 (SEQ ID NO: 249) | 89.9% |
| Hu14H3VLv1 (SEQ ID NO: 250) | 85.0% |
| Hu14H3VLv2 (SEQ ID NO: 251) | 85.0% |

Positions at which canonical, vernier, or interface residues differ between mouse and human acceptor sequences are candidates for substitution. Examples of canonical/CDR interacting residues include Kabat residue L2 in Table 46. Examples of vernier residues include Kabat residue L2 in Table 46. Examples of interface/packing (VH+VL) residues include Kabat residue L87 in Table 46.

The rationales for selection of the positions indicated in Table 45 in the heavy chain variable region as candidates for substitution are as follows.

Heavy Chain Variable Regions

hu14H3VHv1
consists of the CDR-H1, H2, and H3 loops of 14H3-VH grafted onto the framework of QDJ57937-VH. Additionally, contains a substitution G35bS in the CDR-H1

G35bS: is a mutation of a CDR residue. Residue at position H35b is predicted not to be in direct contact with antigen based upon the model. Human germline gene IGHV1-70*04 & IGHJ4*01 (SEQ ID NO:254) has Ser at this position. L35bS is a germ line-aligning mutation.

hu14H3VHv2
reverts all framework substitutions at positions that are key for defining the Chothia canonical classes, are part of the Vernier zone, or localize to the VH/VL domain interface or contribute to structural stability. hu14H3VHv2 incorporates backmutations or substitutions at various positions as listed below to enable assessment of these positions' contributions to antigen-binding affinity and immunogenicity.
reverts all framework substitutions at positions that are key for defining the Chothia canonical classes, are part of the Vernier zone, or localize to the VH/VL domain interface or contribute to structural stability. hu14H3VHv2 incorporates backmutations or substitutions at various positions as listed below to enable assessment of these positions' contributions to antigen-binding affinity and immunogenicity. Also, incorporates backmutations or substitution with most frequent residue at a given position.

M108L: is a germ-line aligning mutation. Leu is in human germ line gene IGHV1-70*04 & IGHJ4*01 (SEQ ID NO:254) at this position.
L113S: is a germ-line aligning mutation. Ser is in human germ line gene IGHV1-70*04 & IGHJ4*01 (SEQ ID NO:254) at this position.

The rationales for selection of the positions indicated in Table 46 in the light chain variable region as candidates for substitution are as follows.

Kappa Light Chain Variable Regions

hu14H3VLv1
consists of the CDR-L1, L2, and L3 loops of 14H3-VL grafted onto the framework of ABC66914 VL. Additionally, hu14H3VLv1 also reverts all framework substitutions at positions that are key for defining the Chothia canonical classes, are part of the Vernier zone, or localize to the VH/VL domain interface contribute to structural stability.

I2V: is a backmutation of a Chothia canonical structure residue, in order to preserve the canonical structure.

Y87F: is a backmutation of a VH/VL interface residue, in order to preserve the heavy chain: light chain interface.

hu14H3VLv2
Retains all reverted framework substitutions at positions that are key for defining the Chothia canonical classes, are part of the Vernier zone, or locate to the VH/VL domain interface. Additionally, hu14H3VLv2 incorporates backmutations or substitution with most frequent residue at a given position and that enhances antibody developability. Mutations in hu14H3VLv2 that are not included in hu14H3VLv1 are:

T7S: is a germ line-aligning mutation. Ser is in is in human germ line gene IGKV2-28*01 & IGKJ2*01 (SEQ ID NO:37) at this position.

L37Q: Leu at this position is immunogenic, therefore, it is replaced with Gln to reduce immunogenicity. L37Q is also a frequency-based mutation. Gln is most frequent residue at this position.

G100Q: is a germ line-aligning mutation. Gln is in human germ line gene IGKV2-28*01 & IGKJ2*01 (SEQ ID NO:37) at this position.

V104L: is a germ line-aligning mutation. Leu is in human germ line gene IGKV2-28*01 & IGKJ2*01 (SEQ ID NO:37) at this position.

The designs based on these human frameworks were:

heavy chain variable regions
>hu14H3VHv1
QVTLKESGPALVKPTQTLTLTCTFSGFSLSTYGMGVSW
IRQPPGKALEWLANIWWDDIKYYNAALKSRLTISKDTS
KNQVVLTMTNMDPVDTATYYCARNVDYWGQGTMVTVSL
>hu14H3VHv2
QVTLKESGPALVKPTQTLTLTCTFSGFSLSTYGMGVSW
IRQPPGKALEWLANIWWDDIKYYNAALKSRLTISKDTS
KNQVVLTMTNMDPVDTATYYCARNVDYWGQGTLVTVSS
kappa light chain variable regions
>hu14H3VL v1
DVVMTQTPLSLPVTPGEPASISCRSSQSLVHSNGNTFL
HWYLQKPGQSPQLLIYKVSNRFSGVPDRFSGSGSGTDF
TLKISRVEAEDVGVYFCSQSTLVPWTFGGGTKVEIK
>hu14H3VLv2
DVVMTQSPLSLPVTPGEPASISCRSSQSLVHSNGNTFL
HWYQQKPGQSPQLLIYKVSNRFSGVPDRFSGSGSGTDF
TLKISRVEAEDVGVYFCSQSTLVPWTFGQGTKLEIK

Humanized sequences are generated using a two-stage PCR protocol that allows introduction of multiple mutations, deletions, and insertions using QuikChange site-directed mutagenesis [Wang, W. and Malcolm, B. A. (1999) BioTechniques 26:680-682).

### Example 13. Mouse 9F5, Mouse 10C12, Mouse 12C4, and Mouse 2D11 Prevent Tau Toxicity in Primary Neurons

Cortical neurons from embryonic day 16-17 were prepared from C57B16/J mouse fetuses, as described in Pillot et al., 1999; Kriem et al., 2005; and Garcia et al., 2010. In brief, dissociated cortical cells were plated (50.000 cells/well) in 48-well plates pre-coated with 1.5 μg/mL polyornithine (Sigma). Cells were cultured in a chemically defined Dulbecco's-modified Eagle's/F12 medium free of serum and supplemented with hormones, proteins and salts. Cultures were kept at 35° C. in a humidified 6% CO₂ atmosphere.

All treatments were done in triplicates at day 6 to 7 in vitro (DIV). Neurons were incubated either with vehicle or with human tau oligomers (1 μM final concentration based on monomers) in the presence of 5 increasing concentrations of the indicated antibody, for 24 h and in a final volume of 140 μL per well.

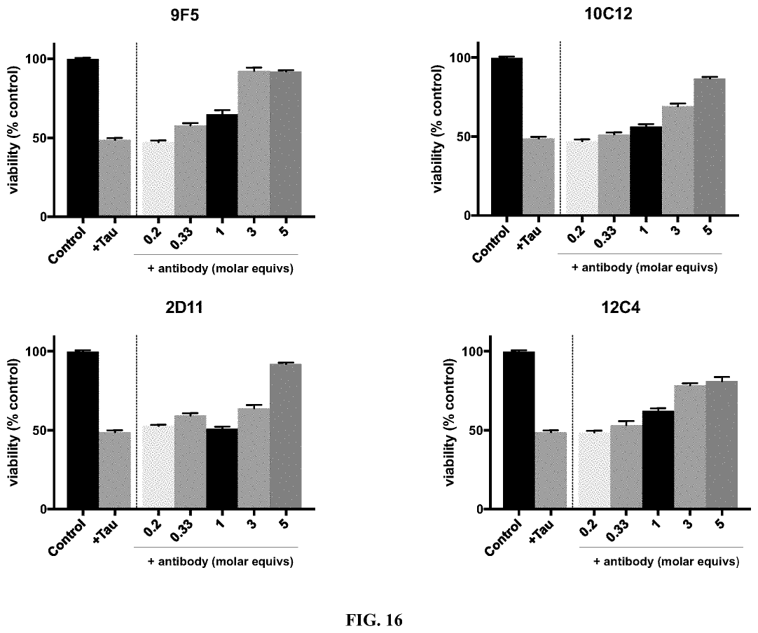

After incubations, neuronal viability was measured using 3-(4,5-Dimethyl-2-thiazolyl)-2,5-diphenyl-2H-tetrazolium bromide (MTT), using published protocols (e.g. Mosmann et al., 1983). Cells were incubated at 35° C. for 1 h with MTT. For that purpose, MTT was solubilized in PBS at 5 mg/mL. 14 μL MTT solution were added to each well. After incubation, medium was removed, and cells were lysed with 150 μL DMSO for 10 minutes and protected from light. After complete solubilization of the formazan product, absorbance was determined at 570 nm. Viability in vehicle control was defined as 100%. Data were expressed as % of viability in vehicle control. Results are shown in Table 57 and FIG. 16.

TABLE 57
MTT assay using 9F5, 10C12, 2D11, and 12C4
All values are cell viability (% of control of
cells not treated with tau oligomers) ± SD

| Antibody | | | | |
|---|---|---|---|---|
| amount | | | | |
| (molar | | | | |
| equivs) | 9F5 | 10C12 | 2D11 | 12C4 |
| 5 | 93.1 ± 1.5 | 86.8 ± 1.7 | 92.2 ± 1.1 | 81.1 ± 4.5 |
| 3 | 92.6 ± 3.4 | 69.2 ± 3 | 64 ± 3.6 | 78.7 ± 2.1 |
| 1 | 64.9 ± 4.5 | 56.4 ± 2.6 | 50.9 ± 2.4 | 62.3 ± 2.9 |
| 0.33 | 58 ± 2.6 | 51.3 ± 2.4 | 59.5 ± 2.3 | 53.2 ± 4.5 |
| 0.2 | 47.4 ± 1.6 | 47.1 ± 2.1 | 52.8 ± 1.1 | 48.6 ± 2.1 |
| 0 | 48.9 ± 2.8 | 48.9 ± 1.9 | 50.9 ± 2.6 | 50.9 ± 3.4 |

All four antibodies demonstrated the ability to protect neurons against tau-induced toxicity. It was noted that 9F5 demonstrated higher potency, with the ability to fully inhibit toxicity at a lower concentration than the other antibodies.

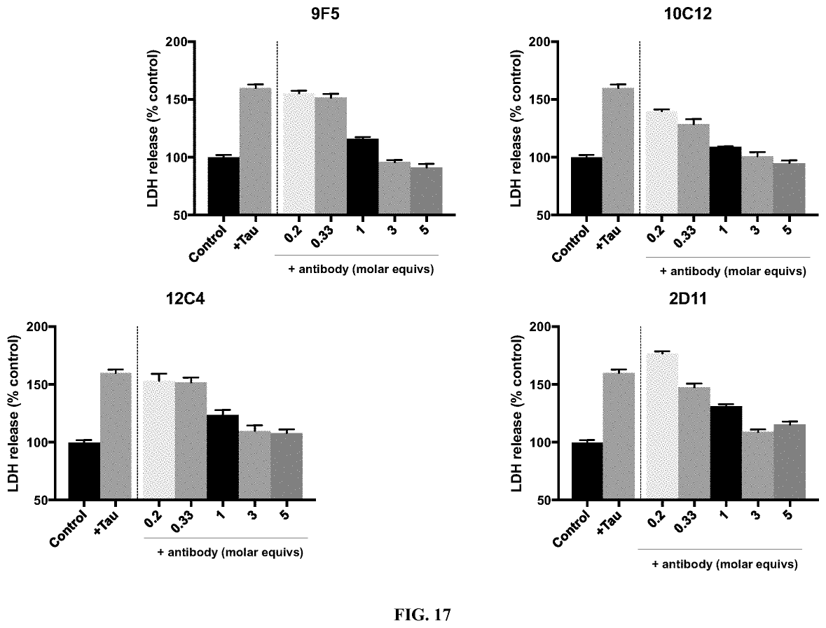

Lactate dehydrogenase (LDH) release is an indicator of cell death. Reduced LDH indicates reduced cell death, resulting from reduced internalization of tau. For the measurement of lactate dehydrogenase (LDH) release, culture medium (110 μL) of each well was transferred into a 1.5 mL Eppendorf tube and replaced by fresh medium for the MTT assay. Collected medium was centrifuged at 800 g for five minutes and the supernatant (100 μL of cell-free culture medium) was transferred into a 48-well plate stored at 4° C. and protected from light for further analysis. The quantification of LDH in culture medium was performed according to manufacturer's recommendations (Cytotoxicity Detection Kit [LDH], Roche Ref 11 644 793 001). Results are shown in Table 58 and FIG. 17.

TABLE 58
LDH assay using 9F5, 10C12, 2D11, and 12C4
All values are LDF released (% of control of
cells not treated with tau oligomers) ± SD

| Antibody | | | | |
|---|---|---|---|---|
| amount | | | | |
| (molar | | | | |
| equivs) | 9F5 | 10C12 | 2D11 | 12C4 |
| 5 | 91.4 ± 5.1 | 95.1 ± 3.9 | 115.4 ± 4.5 | 108.2 ± 5.2 |
| 3 | 95.9 ± 2.9 | 101.1 ± 5.8 | 109 ± 3.8 | 109.9 ± 8.2 |
| 1 | 116.0 ± 2.6 | 109 ± 1.1 | 131.1 ± 3.3 | 123.7 ± 7.5 |
| 0.33 | 151.5 ± 5.9 | 128.6 ± 7.6 | 147.7 ± 5.3 | 151.9 ± 7.1 |
| 0.2 | 155.4 ± 3.8 | 140 ± 2.7 | 177 ± 3.0 | 153.4 ± 10.3 |
| 0 | 158.1 ± 4.5 | 145.1 ± 3.9 | 159.4 ± 7.8 | 160.1 ± 4.1 |

All four antibodies demonstrated the ability to protect neurons against tau-induced toxicity. It was noted that 9F5 demonstrated higher potency, with the ability to fully inhibit toxicity at a lower concentration than the other antibodies, and even reduced LDH release below baseline at the highest concentration added.

### REFERENCES

Pillot, T., Drouet, B., Queillé, S., et al., The nonfibrillar amyloid beta-peptide induces apoptotic neuronal cell death: involvement of its C-terminal fusogenic domain. J Neurochem. 73(4):1626-34 (1999).
Kriem, B., Sponne, I., Fifre, A., et al., Cytosolic phospholipase A2 mediates neuronal apoptosis induced by soluble oligomers of the amyloid-beta peptide. FASEB J. 19(1):85-7 (2005).
Garcia, P., Youssef, I., Utvik, J. K., et al, Ciliary neurotrophic factor cell-based delivery prevents synaptic impairment and improves memory in mouse models of Alzheimer's disease. J Neurosci. 30(22): 7516-27 (2010).
Mosmann, T., Rapid colorimetric assay for cellular growth and survival: application to proliferation and cytotoxicity assays. J. Immunol. Methods., 65, 55-63 (1983).

### Example 14. Surface Plasmon Resonance (SPR) Analysis of Mouse 9F5, Mouse 10C12, Mouse 14H3, Mouse 17C12, Mouse 2D11, and Mouse 12C4

Surface plasmon resonance (SPR) analysis using Biacore was performed by immobilizing an anti-mouse (GE Lifesciences) antibody to a CM3 chip with EDC/NHS. Antibodies were then captured to the anti-mouse surface to equivalent levels. Regeneration was performed with 2 30 s injections of 10 mM Glycine pH 1.7. Various concentrations ranging from 500 to 0.122 nM, diluted in 3-fold dilutions, of recombinant human 4R0N tau were flowed over the sensor for a 150s association phase and 300 s dissociation phase. Data was blank subtracted to both an irrelevant sensor not containing test antibody to account for irrelevant binding to the prepared sensor surface, and to the mobile phase vehicle to account for the dissociation of test antibody from the anti-mouse capture. Data was then analyzed with onboard evaluation software using a global 1:1 fit. Results are shown in Table 51. All antibodies displayed similar binding kinetics with Kd values within 200 pM of each other; kinetic profiles featured rapid association and slow dissociation phases.

TABLE 51
Kinetics parameters of mouse 9F5, mouse 10C12, mouse
14H3, Mouse 17C12, mouse 2D11, and mouse 12C4

| Antibody | kₐ (M⁻¹ s⁻¹) | k_{d} (s⁻¹) | K_{d} (M) |
|---|---|---|---|
| 9F5 | 6.39e+6 | 2.24e−3 | 3.50e−10 |
| 10C12 | 6.53e+6 | 2.45e−3 | 3.75e−10 |
| 14H3 | 7.60e+6 | 1.53e−3 | 2.02e−10 |
| 17C12 | 4.03e+6 | 1.94e−3 | 4.82e−10 |
| 2D11 | 6.93e+6 | 2.21e−3 | 3.18e−10 |
| 12C4 | 5.69e+6 | 2.06e−3 | 3.63e−10 |

### Example 15. Mouse 9F5, mouse 10C12, Mouse 14H3, Mouse 17C12, Mouse 2D11, and Mouse 12C4 Detect Tau in Samples from Alzheimer's Disease Patients: Western Blotting Analysis

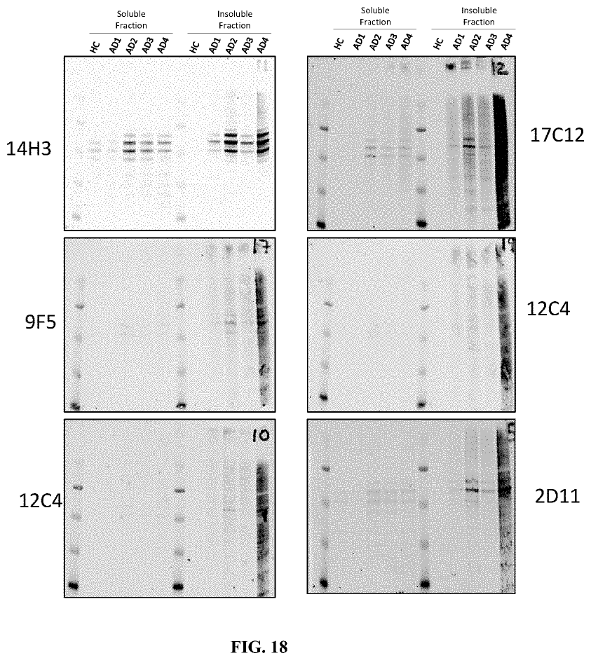

Fresh frozen frontal cortical samples were obtained from brains of one healthy control (HC) and four Alzheimer's disease (AD) patients. Samples were homogenized in10 volumes of RIPA buffer containing protease inhibitors, and centrifuged for 15 minutes at 16,000×g at 4° C. The supernatants were removed to generate the soluble fraction. Pellets were resuspended in 1× PBS containing 1% Sarkosyl and homogenized. Sarkosyl homogenates were centrifuged for 60 minutes at 100,000×g at 4° C., and the supernatants and pellets were separated. Pellets were resuspended in one volume of 2% SDS to generate the insoluble fraction. Resulting soluble and insoluble fractions were resolved by SDS-PAGE, and immunoblotted with indicated antibodies at 0.5 ug/mL, washed, and probed with goat anti-mouse secondary antibodies conjugated to IRDye-800 (LiCor). After washing, blots were scanned using a LiCor scanner. Results are shown in FIG. 18. All antibodies specifically detected tau contained in the insoluble fractions of AD patients, and detected tau to varying degrees in the soluble fractions.

### Example 16. Immunoprecipitation Analysis of Insoluble Extract from Alzheimer's Disease Patient with Mouse 9F5, Mouse 10C12, Mouse 14H3, Mouse 17C12, Mouse 2D11, and Mouse 12C4

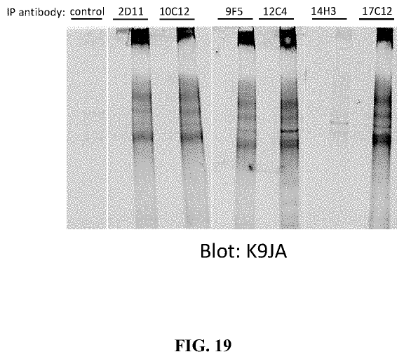

In order to test antibody capacity to capture tau aggregates, the sarkosyl-insoluble fraction from one Alzheimer's disease (AD) patient and one normal control was incubated with indicated antibodies. Magnetic beads conjugated to goat anti-mouse antibody (Life Technologies) were then added to the extract/antibody mixtures to allow capture of antibody/antigen complexes. Beads were washed thoroughly, boiled in SDS-PAGE running buffer containing DTT, and the beads were removed. Resulting fractions were resolved by SDS-PAGE, and immunoblotted with the polyclonal antibody K9JA to detect all immunocaptured tau species. Results are shown in FIG. 19. Alternating lanes are immunoprecipitates from normal and AD tissue extracts. Molecular weight markers are indicated at the left. All antibodies captured insoluble tau from AD patients to varying degrees, and demonstrated the ability to capture large insoluble high-mass aggregates. Of the antibodies tested, 9F5, 2D11, 17C12, 10C12 and 12C4 displayed similar profiles, with strong immunoreactivity present in the 50-60 kDa range as well as in the stacking gel. 14H3 capture also resulted in similar, though less abundant, tau species. These data indicate that all antibodies tested are able to bind a similar array of tau forms.

### Example 17. Ability of Humanized 9F5 Variants to Withstand Agitation Stress

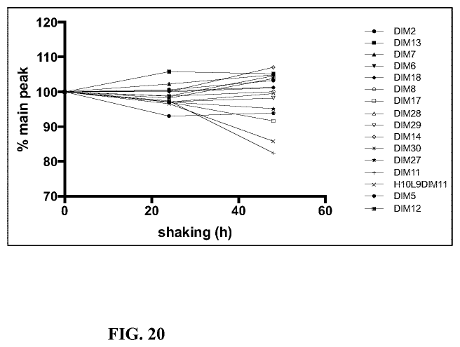

Developability characteristics of humanized variants of 9F5 were tested for their ability to withstand aggregation induced by agitation stress. Antibodies were buffer-exchanged into histidine buffer (25 mM Histidine, pH 6) at 1 mg/mL, and underwent agitation at 1500 rpm at room temperature for 48 h. Samples were withdrawn at 0, 24 and 48 h, filtered through a 0.22 micron filter and analyzed by analytical size exclusion chromatography (SEC). For each antibody, the total area of the main peaks for each timepoint were normalized to the area of the main peak at t=0 h, and antibodies were ranked based on the loss of antibody to aggregation over time. Results are shown in FIG. 20 and Table 52. Abbreviations for names of 9F5 humanized variants used in FIG. 20 are as described in the second column of Table 52. While no single point mutation can be attributed as causing sensitivity to agitation stress, some combinations of mutations contribute to the lack of resistance. Of note, the combinations L27cG/L37G/M51G/L54R/L92I (DIM11), L27cS/L37G/M51GL54T (DIM17), and L27cG/L37Q/M51G/L54R (DIM5) result in increased sensitivity to agitation, while the combinations, for example, L27cS/L37Q/M51G/L54R (DIM2), L27cG/L37G/M51G/L54T (DIM14), and L27cG/L37G/M51G/L54R (DIM13) result in resistance to agitation stress. These results suggest the generation of specific residue combinations in the humanized variants that would be particularly likely to survive the rigors of manufacturing, handling, and shipment.

TABLE 52
Ability of humanized 9F5 variants to withstand agitation stress

| | | Monomer at 24 h | Monomer 48 h |
|---|---|---|---|
| Rank | 9F5 Humanized Variant | shaking (% t = 0) | shaking (% t = 0) |
| 1 | DIM2 [hu9F5VHv9/hu9F5VLv8_DIM2 | 100.6 | 103.3 |
| | SEQ ID NO: 127/SEQ ID NO: 133] | | |
| 2 | DIM14 [hu9F5VHv9/hu9F5VLv8_DIM14 | 100.1 | 107.1 |
| | SEQ ID NO: 127/SEQ ID NO: 145] | | |
| 3 | DIM13 [hu9F5VHv9/hu9F5VLv8_DIM13 | 105.8 | 105.2 |
| | SEQ ID NO: 127/SEQ ID NO: 144] | | |
| 4 | DIM7 [hu9F5VHv9/hu9F5VLv8_DIM7 | 102.3 | 105 |
| | SEQ ID NO: 127/SEQ ID NO: 138] | | |
| 5 | DIM6 [hu9F5VHv9/hu9F5VLv8_DIM6 | 98.8 | 104.6 |
| | SEQ ID NO: 127/SEQ ID NO: 137] | | |
| 6 | DIM12 [hu9F5VHv9/hu9F5VLv8_DIM12 | 98.2 | 103.8 |
| | SEQ ID NO: 127/SEQ ID NO: 143] | | |
| 7 | DIM18 [hu9F5VHv9/hu9F5VLv8_DIM18 | 100.3 | 101.3 |
| | SEQ ID NO: 127/SEQ ID NO: 149] | | |
| 8 | DIM30 [hu9F5VHv9/hu9F5VLv8_DIM30 | 100 | 101.2 |
| | SEQ ID NO: 127/SEQ ID NO: 161] | | |
| 9 | DIM8 [hu9F5VHv9/hu9F5VLv8_DIM8 | 98.8 | 100.1 |
| | SEQ ID NO: 127/SEQ ID NO: 139] | | |
| 10 | DIM28 [hu9F5VHv9/hu9F5VLv8_DIM28 | 97.1 | 99.5 |
| | SEQ ID NO: 127/SEQ ID NO: 159] | | |
| 11 | DIM29 [hu9F5VHv9/hu9F5VLv8_DIM29 | 97.2 | 98.2 |
| | SEQ ID NO: 127/SEQ ID NO: 160] | | |
| 12 | DIM27 [hu9F5VHv9/hu9F5VLv8_DIM27 | 97.1 | 95.2 |
| | SEQ ID NO: 127/SEQ ID NO: 158] | | |
| 13 | DIM5 [hu9F5VHv9/hu9F5VLv8_DIM5 | 93.1 | 93.9 |
| | SEQ ID NO: 127/SEQ ID NO: 136] | | |
| 14 | DIM17 [hu9F5VHv9/hu9F5VLv8_DIM17 | 97.1 | 91.6 |
| | SEQ ID NO: 127/SEQ ID NO: 148] | | |
| 15 | H10L9-DIM11 | 96.6 | 85.8 |
| | [hu9F5VHv10/hu9F5VLv9_DIM11 | | |
| | SEQ ID NO: 128/SEQ ID NO: 168] | | |
| 16 | DIM11 [hu9F5VHv9/hu9F5VLv8_DIM11 | 97.4 | 82.4 |
| | SEQ ID NO: 127/SEQ ID NO: 142] | | |

### Example 18. Ability of Humanized 9F5 Variants to Withstand Low pH Stress

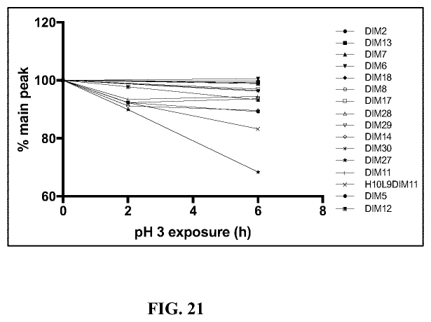

Developability characteristics of humanized variants of 9F5 were tested for their ability to withstand low pH exposure, as would be encountered during viral inactivation steps of manufacturing. Antibodies were buffer-exchanged into acetate buffer (10 mM acetate, pH 3) at 1 mg/mL, and incubated at room temperature for 6 h. Samples were withdrawn at 0, 2 and 6 h, filtered through a 0.22 micron filter and analyzed by analytical size exclusion chromatography (SEC). For each antibody, the total area of the main peaks for each timepoint were normalized to the area of the main peak at t=0 h, and antibodies were ranked based on the loss of antibody to aggregation over time. Results are shown in FIG. 21 and Table 53. Abbreviations for names of 9F5 humanized variants used in FIG. 21 are as described in the second column of Table 53. The combinations L27cG/L37G/M51G/L54R/L92I (DIM11), L27cG/L37Q/M51G/L54R (DIM5), and L27cG/L37G/M51G/L54T (DIM14) result in increased sensitivity to low pH stress as evidenced by a decrease of >5% of the monomer peak at 6 h, while the combinations, for example, L27cS/L37Q/M51G/L54R (DIM2), L27cD/L37Q/M51G/L54R (DIM6), and L27cG/L37G/M51G/L54R (DIM13) result in resistance to to low pH stress. Of note, the inclusion of mutation L27cD resulted in low pH-resistant variants DIM18, DIM6 and DIM7. These results suggest the generation of specific residue combinations in the humanized variants that would be particularly likely to survive the rigors of manufacturing, in particular the inclusion of low pH incubations commonly included during purification.

TABLE 53
Ability of humanized 9F5 variants to withstand low pH exposure

| | | Monomer at 2 h | Monomer at 6 h |
|---|---|---|---|
| Rank | h9F5 Variant | pH 3 (% t = 0) | pH 3 (% t = 0) |
| 1 | DIM6 [hu9F5VHv9/hu9F5VLv8_DIM6 | 100.8 | 100.6 |
| | SEQ ID NO: 127/SEQ ID NO: 137] | | |
| 2 | DIM2 [hu9F5VHv9/hu9F5VLv8_DIM2 | 100.6 | 99.6 |
| | SEQ ID NO: 127/SEQ ID NO: 133] | | |
| 3 | DIM13 [hu9F5VHv9/hu9F5VLv8_D]IM13 | 97.4 | 99.3 |
| | SEQ ID NO: 127/SEQ ID NO: 144 | | |
| 4 | DIM8 [hu9F5VHv9/hu9F5VLv8_DIM8 | 99.2 | 99.2 |
| | SEQ ID NO: 127/SEQ ID NO: 139] | | |
| 5 | DIM7 [hu9F5VHv9/hu9F5VLv8_DIM7 | 99.6 | 98.9 |
| | SEQ ID NO: 127/SEQ ID NO: 138] | | |
| 6 | DIM28 [hu9F5VHv9/hu9F5VLv8_DIM28 | 99.1 | 98.9 |
| | SEQ ID NO: 127/SEQ ID NO: 159] | | |
| 7 | DIM17 [hu9F5VHv9/hu9F5VLv8_DIM17 | 96.5 | 97 |
| | SEQ ID NO: 127/SEQ ID NO: 148] | | |
| 8 | DIM18 [hu9F5VHv9/hu9F5VLv8_DIM18 | 97.2 | 96.5 |
| | SEQ ID NO: 127/SEQ ID NO: 149] | | |
| 9 | DIM30 [hu9F5VHv9/hu9F5VLv8_DIM30 | 96.5 | 96.3 |
| | SEQ ID NO: 127/SEQ ID NO: 161] | | |
| 10 | DIM11 [hu9F5VHv9/hu9F5VLv8_DIM11 | 93.6 | 94.5 |
| | SEQ ID NO: 127/SEQ ID NO: 142] | | |
| 11 | DIM29 [hu9F5VHv9/hu9F5VLv8_DIM29 | 94.0 | 93.6 |
| | SEQ ID NO: 127/SEQ ID NO: 160] | | |
| 12 | DIM12 [hu9F5VHv9/hu9F5VLv8_DIM12 | 93.9 | 93.3 |
| | SEQ ID NO: 127/SEQ ID NO: 143] | | |
| 13 | DIM14 [hu9F5VHv9/hu9F5VLv8_DIM14 | 93.5 | 89.6 |
| | SEQ ID NO: 127/SEQ ID NO: 145] | | |
| 14 | DIM5 [hu9F5VHv9/hu9F5VLv8_DIM5 | 93.6 | 89.2 |
| | SEQ ID NO: 127/SEQ ID NO: 136] | | |
| 15 | H10L9-DIM11 | 93.2 | 83.3 |
| | [hu9F5VHv10/hu9F5VLv9_DIM11 | | |
| | SEQ ID NO: 128/SEQ ID NO: 168] | | |
| 16 | DIM27 [hu9F5VHv9/hu9F5VLv8_DIM27 | 91.3 | 68.4 |
| | SEQ ID NO: 127/SEQ ID NO: 158] | | |

### Example 19. PEG Precipitation with 9F5 Variants

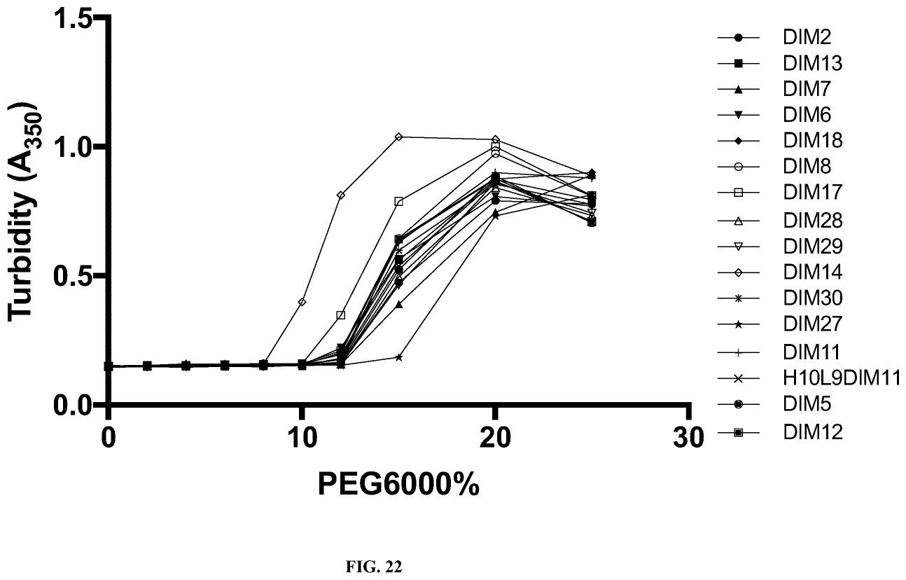

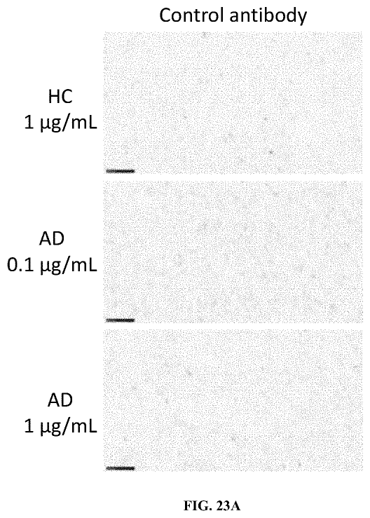

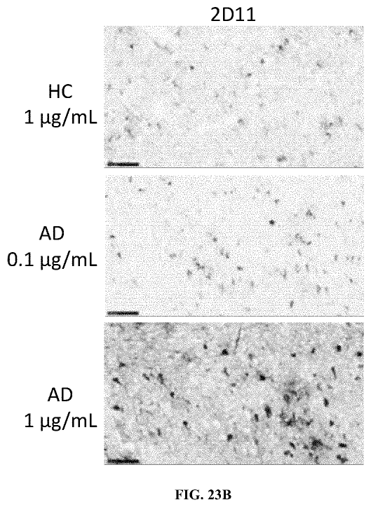

PEG precipitation was utilized to determine the relative propensity of different humanized 9F5 variants to aggregate under simulated high-concentration conditions. Antibodies were buffer-exchanged into histidine buffer (25 mM histidine, pH 6), and added to a microplate containing indicated amounts of PEG6000 to a final protein concentration of 1 mg/mL, and incubated at room temperature for 1 h. After incubation, turbidity was measured at an absorbance of 350 nM. Results are shown in Tables 59-60 and FIG. 22.

TABLE 59
PEG precipitation of 9F5 variants DIM2, DIM13,
DIM7, DIM6, DIM18, DIM8, DIM17, and DIM28

| PEG6000 % | DIM2 | DIM13 | DIM7 | DIM6 | DIM18 | DIM8 | DIM17 | DIM28 |
|---|---|---|---|---|---|---|---|---|
| 25 | 0.7784 | 0.7944 | 0.8912 | 0.7688 | 0.901 | 0.8085 | 0.811 | 0.733 |
| 20 | 0.7904 | 0.8737 | 0.7454 | 0.8063 | 0.8743 | 0.9733 | 1.001 | 0.842 |
| 15 | 0.4729 | 0.5612 | 0.3895 | 0.5658 | 0.6362 | 0.6438 | 0.7885 | 0.4952 |
| 12 | 0.1578 | 0.1952 | 0.1563 | 0.2206 | 0.2022 | 0.1592 | 0.3472 | 0.1586 |
| 10 | 0.1554 | 0.1582 | 0.1512 | 0.1541 | 0.1541 | 0.1538 | 0.1582 | 0.1583 |
| 8 | 0.1607 | 0.1546 | 0.15 | 0.15 | 0.1508 | 0.1506 | 0.1556 | 0.1591 |
| 6 | 0.1538 | 0.1519 | 0.1486 | 0.1564 | 0.1494 | 0.1502 | 0.1541 | 0.157 |
| 4 | 0.1526 | 0.1516 | 0.1526 | 0.1479 | 0.1462 | 0.1504 | 0.1513 | 0.1587 |
| 2 | 0.1495 | 0.1511 | 0.1523 | 0.148 | 0.1467 | 0.1478 | 0.1497 | 0.1518 |
| 0 | 0.1464 | 0.1481 | 0.1501 | 0.1458 | 0.1451 | 0.1471 | 0.1477 | 0.1496 |

TABLE 60
PEG precipitation of 9F5 variants DIM29, DIM14,
DIM30, DIM27, DIM11, H10L9DIM11, DIM5, and DIM12

| PEG6000 % | DIM29 | DIM14 | DIM30 | DIM27 | DIM11 | H10L9DIM11 | DIM5 | DIM12 |
|---|---|---|---|---|---|---|---|---|
| 25 | 0.7422 | 0.8867 | 0.7759 | 0.8138 | 0.8785 | 0.7122 | 0.7042 | 0.7078 |
| 20 | 0.8623 | 1.0284 | 0.8549 | 0.732 | 0.8999 | 0.8663 | 0.8869 | 0.8767 |
| 15 | 0.4642 | 1.0377 | 0.598 | 0.1843 | 0.6288 | 0.5383 | 0.5228 | 0.6414 |
| 12 | 0.1564 | 0.8123 | 0.1752 | 0.1536 | 0.1802 | 0.1755 | 0.1652 | 0.2092 |
| 10 | 0.1539 | 0.3975 | 0.1545 | 0.1525 | 0.1522 | 0.1575 | 0.1548 | 0.159 |
| 8 | 0.1542 | 0.1564 | 0.1475 | 0.1532 | 0.1514 | 0.1535 | 0.1549 | 0.1572 |
| 6 | 0.1518 | 0.1505 | 0.1492 | 0.1497 | 0.1486 | 0.1503 | 0.157 | 0.1559 |
| 4 | 0.1494 | 0.1496 | 0.1483 | 0.1469 | 0.1484 | 0.149 | 0.1523 | 0.153 |
| 2 | 0.1475 | 0.1485 | 0.1473 | 0.1469 | 0.1485 | 0.1482 | 0.1507 | 0.1526 |
| 0 | 0.1482 | 0.1537 | 0.1499 | 0.148 | 0.1484 | 0.1465 | 0.1487 | 0.149 |

Abbreviations for names of 9F5 humanized variants used in Tables 59-60 and FIG. 22 are as described in the second column of Table 53. Antibodies were qualitatively ranked by the PEG6000 concentration that induced the onset of turbidity. DIM14 [hu9F5VHv9/hu9F5VLv8_DIM14; SEQ ID NO: 127/SEQ ID NO: 145] and DIM17 [hu9F5VHv9/hu9F5VLv8_DIM17; SEQ ID NO: 127/SEQ ID NO: 148] displayed aggregation at a lower PEG6000 concentration compared with most antibodies, and DIM27 [hu9F5VHv9/hu9F5VLv8_DIM27; SEQ ID NO: 127/SEQ ID NO: 158] displayed aggregation at a higher PEG6000 concentration compared with most antibodies. The combinations L27cG/L37G/M51G/L54T/L92I (DIM14) and L27cS/L37G/M51G/L54T (DIM17) resulted in decreased solubility in the presence of increasing amounts of PEG6000, as evidenced by the earlier onset of turbidity. In contrast, the combination L37Q/M51G/L54R combined with the original leucine at position L27c(DIM27), result in increased solubility in the presence of increasing amounts of PEG6000. These results suggest the generation of specific residue combinations in the humanized variants that would be particularly likely to survive formulation at the high concentrations of antibody typically required for clinical use.

### Example 20. Immunohistochemistry Analysis of Human Brain Samples Using Control, Mouse 2D11, Mouse 9F5, Mouse 12C4, Mouse 14H3, and Mouse 17C12

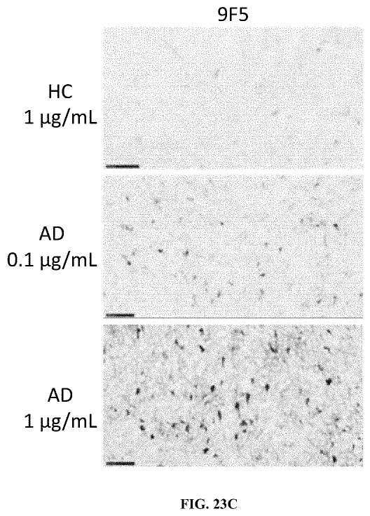

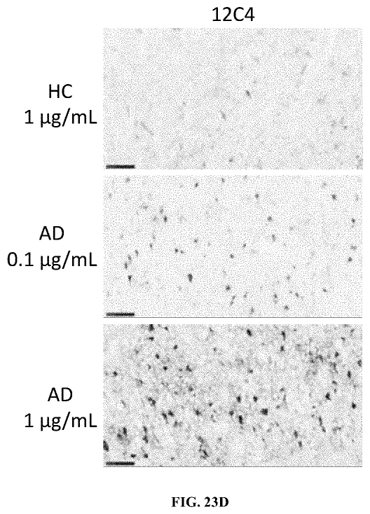

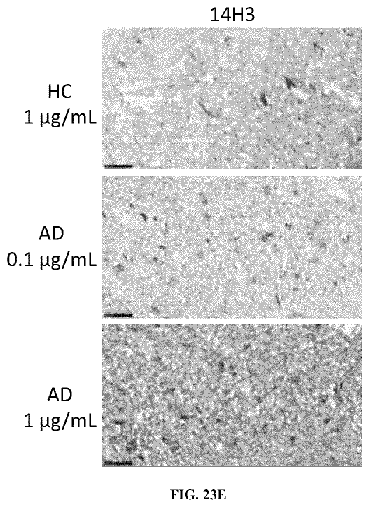

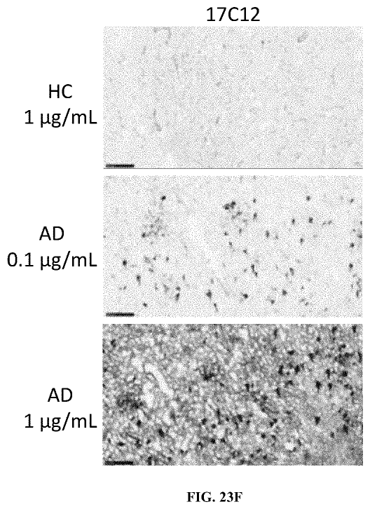

Fresh-frozen human brain samples were embedded in OCT, and cut using a cryostat to generate 10 μm sections. Sections were fixed to slides using 10% neutral-buffered formalin for 10 minutes at 4° C., and were treated with glucose oxidase to block endogenous peroxidases. Sections were stained using indicated primary antibodies using the BOND Polymer Refine Detection system (Leica), which contains DAB chromagen and hematoxylin counterstain. Sections were then dehydrated in an increasing series of ethanol concentrations and xylene before coverslipping. Results are shown in FIGS. 23A-F. As expected, nonimmune control antibody displayed little binding to either normal control or Alzheimer's disease samples at both concentrations tested. In contrast, anti-MTBR antibodies demonstrated abundant binding to pathological features present in Alzheimer's disease, including dystrophic neurites and neurofibrillary tangles. This binding displayed dose dependence, with stronger binding evident at higher antibody concentrations. A subset of antibodies displayed little binding to normal control sections, notably 9F5 and 12C4 and 17C12. 14H3 and 2D11 displayed binding to normal control tissue, indicating subtle differences in specificity towards tau present in these samples. For 14H3, the lessened selectivity towards AD compared to control samples mirrors the difference in immunoreactivity detected in immunoprecipitation experiments, indicating that this antibody binds normal forms of tau to a greater degree than 2D11, 9F5, 12C4 and 17C12.

### Example 21. Exemplary CDRs

Exemplary CDRs of antibodies of the invention are in Table 54.

TABLE 54
Exemplary CDRs

| | | | Exemplary VH |
|---|---|---|---|
| | | SEQ | or VL that |
| CDR and | CDR Amino Acid | ID | CDR is |
| Definition | Sequence | NO: | present in |
| Kabat/Chothia | GFNIKDDYMN | 8 | Mouse 9F5 VH |
| composite CDR-H1 | | | |
| Kabat CDR-H2 | WIDPENGDTEY | 9 | Mouse 9F5 VH |
| | ASKFQG | | |
| Kabat CDR-H3 | SNG | 10 | Mouse 9F5 VH |
| Kabat CDR-L1 | RSSKSLLHSN | 12 | Mouse 9F5 VL |
| | GITYLY | | |
| Kabat CDR-L2 | QMSNLAS | 13 | Mouse 9F5 VL |
| Kabat CDR-L3 | AQNLELPLT | 14 | Mouse 9F5 VL |
| Kabat CDR-H1 | DDYMN | 40 | Mouse 9F5 VH |
| Chothia CDR-H1 | GFNIKDD | 41 | Mouse 9F5 VH |
| Chothia CDR-H2 | DPENGD | 42 | Mouse 9F5 VH |
| AbM CDR-H2 | WIDPENGDTE | 43 | Mouse 9F5 VH |
| Contact CDR-H1 | KDDYMN | 44 | Mouse 9F5 VH |
| Contact CDR-H2 | WIGWIDPENGDTE | 45 | Mouse 9F5 VH |
| Contact CDR-H3 | TTSN | 46 | Mouse 9F5 VH |
| Contact CDR-L1 | ITYLYWY | 47 | Mouse 9F5 VL |
| Contact CDR-L2 | LLIYQMSNLA | 48 | Mouse 9F5 VL |
| Contact CDR-L3 | AQNLELPL | 49 | Mouse 9F5 VL |
| Kabat-Chothia | GFTIKDDYMN | 50 | hu9F5VHv4, |
| Composite CDR-H1 | | | hu9F5VHv5, |
| | | | and |
| | | | hu9F5VHv6 |
| Kabat CDR-H2 | WVDPEDGETEY | 51 | hu9F5VHv5, |
| | ASKFQG | | hu9F5VHv6, |
| | | | and |
| | | | hu9F5VHv7 |
| Kabat CDR-H2 | WVDPENGDTEY | 52 | hu9F5VHv8 |
| | ASKFQG | | |
| Kabat CDR-Ll | RSSKSLLHSNG | 53 | hu9F5VLv5 |
| | YTYLY | | and |
| | | | hu9F5VLv6 |
| Kabat CDR-L1 | RSSKSLLHSNG | 54 | hu9F5VLv7 |
| | INYLY | | |
| Kabat CDR-L2 | QGSNRAS | 55 | hu9F5VLv4, |
| | | | hu9F5VLv5, |
| | | | hu9F5VLv6, |
| | | | hu9F5VLv7, |
| | | | SEQ ID NO: |
| | | | 133, 135, |
| | | | 136, 137, |
| | | | 142, 143, |
| | | | 144, 149, |
| | | | 158, 159, |
| | | | 163, 164, |
| | | | 165, 168, |
| | | | 169 |
| Kabat CDR-L1 | RSSKSDLHSN | 172 | hu9F5VLv2_ |
| | GITYLY | | L27Bd, |
| | | | SEQ ID |
| | | | NO: 71 |
| Kabat CDR-L1 | RSSKSTLHSN | 173 | hu9F5VLv2_ |
| | GITYLY | | L27bT, |
| | | | SEQ ID |
| | | | NO: 79 |
| Kabat CDR-L1 | RSSKSQLHSN | 174 | hu9F5VLv2_ |
| | GITYLY | | L27bQ, |
| | | | SEQ ID |
| | | | NO: 81 |
| Kabat CDR-L1 | RSSKSLDHSNGITYLY | 175 | hu9F5VLv2_L27cD, SEQ ID NO: 63, |
| | | | hu9F5VLv8_DIM6, SEQ ID NO: 137, |
| | | | hu9F5VLv8_D1M7, SEQ ID NO: 138, |
| | | | and hu9F5VLv8_DIM18, SEQ ID NO: 149 |
| Kabat CDR-L1 | RSSKSLGHSNGITYLY | 176 | hu9F5VLv2_L27cG, SEQ ID NO: 64, |
| | | | hu9F5VLv8_DIMS, SEQ ID NO: 136, |
| | | | hu9F5VLv8_DIM8, SEQ ID NO: 139, |
| | | | hu9F5VLv8_D1M9, SEQ ID NO: 140, |
| | | | hu9F5VLv8_DIM11, SEQ ID NO: 142, |
| | | | hu9F5VLv8_DIM12, SEQ ID NO: 143, |
| | | | hu9F5VLv8_DIM13, SEQ ID NO: 144, |
| | | | hu9F5VLv8_D1M14, SEQ ID NO: 145, |
| | | | hu9F5VLv8_DIM15, SEQ ID NO: 146, |
| | | | hu9F5VLv8_DIM16, SEQ ID NO: 147, |
| | | | hu9F5VLv8_D1M23, SEQ ID NO, 154, |
| | | | hu9F5VLv8_DIM24, SEQ ID NO: 155, |
| | | | hu9F5VLv8_DIM25, SEQ ID NO: 156, |
| | | | hu9F5VLv9_DIM5, SEQ ID NO: 165, |
| | | | hu9F5VLv9_DIMS, SEQ ID NO: 166, |
| | | | hu9F5VLv9_DIM11, SEQ ID NO: 168, |
| | | | and |
| | | | hu9F5VLv9_DIM13, SEQ ID NO: 169 |
| Kabat CDR-L1 | RSSKSLSHSNGITYLY | 177 | hu9F5VLv2_L27cS, SEQ ID NO: 65, |
| | | | hu9F5VLv8_DIM1, SEQ ID NO: 132, |
| | | | hu9F5VLv8_DIM2, SEQ ID NO: 133, |
| | | | hu9F5VLv8_DIM3, SEQ ID NO: 134, |
| | | | hu9F5VLv8_DIM4, SEQ ID NO: 135, |
| | | | hu9F5VLv8_DIM10, SEQ ID NO: 141, |
| | | | hu9F5VLv8_DIM17, SEQ ID NO: 148, |
| | | | hu9F5VLv8_DIM19, SEQ ID NO, 150, |
| | | | hu9F5VLv8_DIM20, SEQ ID NO: 151, |
| | | | hu9F5VLvS D1M21, SEQ ID NO: 152,, |
| | | | hu9F5VLv8_DIM22, SEQ ID NO 153, |
| | | | hu9F5VLv8_DIM28, SEQ ID NO: 159, |
| | | | hu9F5VLv8_DIM29, SEQ ID NO: 160, |
| | | | hu9F5VLv8_DIM30, SEQ I NO 161, |
| | | | hu9F5VLv9_DIM1, SEQ ID NO: 162, |
| | | | hu9F5VLv9_D1M2, SEQ ID NO: 163, |
| | | | hu9F5VLv9_DIM4, SEQ ID NO: 164, |
| | | | hu9F5VLv9_DIM10, SEQ ID NO: 167, |
| | | | hu9F5VLv9_DIM19, SEQ ID NO: 170, |
| | | | and hu9F5VLv9_D1M20, SEQ ID NO: 171 |
| Kabat CDR-L1 | RSSKSLEHSNGITYLY | 178 | hu9F5VLv2_L27cE, SEQ ID NO:66 |
| Kabat CDR-L1 | RSSKSLTHSNGITYLY | 179 | hu9F5VLv2_L27cT, SEQ ID NO:69 |
| Kabat CDR-L1 | RSSKSLNHSNGITYLY | 180 | hu9F5VLv2_L27cN, SEQ ID NO:70 |
| Kabat CDR-L1 | RSSKSLAHSNGITYLY | 181 | hu9F5VLv2_L27cA, SEQ ID NO:74 |
| Kabat CDR-Ll | RSSKSLPHSNGITYLY | 182 | hu9F5VLv2_L27cP, SEQ ID NO:83 |
| Kabat CDR-L1 | RSSKSLIHSNGITYLY | 183 | Hu9F5VLv8_DIM26, SEQ ID NO: 157 |
| Kabat CDR-L1 | RSSKSLLHSNGETYLY | 184 | hu9F5VLv2_I30E, SEQ ID NO: 67 |
| Kabat CDR-Ll | RSSKSLLHSNGKTYLY | 185 | hu9F5VLv2_I30K, SEQ ID NO:68 |
| Kabat CDR-L1 | RSSKSLLHSNGGTYLY | 186 | hu9F5VLv2_I30G, SEQ ID NO: 72 |
| Kabat CDR-L1 | RSSKSLLHSNGQTYLY | 187 | hu9F5VLv2_I30Q, SEQ ID NO: 78 |
| Kabat CDR-L1 | RSSKSLLHSNGIGYLY | 188 | hu9F5VLv2 T3IG, SEQ ID NO:80 |
| Kabat CDR-L1 | RSSKSLLHSNGITYNY | 189 | hu9F5VLv2_L33N, SEQ ID NO:73 |
| Kabat CDR-L1 | RSSKSLLHSNGITYTY | 190 | hu9F5VLv2_L33T, SEQ ID NO:75 |
| Kabat CDR-L1 | RSSKSLLHSNGITYSY | 191 | hu9F5VLv2_L33S, SEQ ID NO:76 |
| Kabat CDR-L1 | RSSKSLLHSNGITYRY | 192 | hu9F5VLv2_L33R, SEQ ID NO:77 |
| Kabat CDR-L1 | RSSKSLLHSNGITYGY | 193 | hu9F5VLv2_L33G, SEQ ID NO:82 |
| Kabat CDR-L2 | QESNLAS | 194 | hu9F5VLv2_M51E, SEQ ID NO:61 |
| Kabat CDR-L2 | QDSNLAS | 195 | hu9F5VLv2_M51D, SEQ ID NO:62 |
| Kabat CDR-L2 | OGSNLAS | 196 | hu9F5VLv8_DIM30, SEQ ID NO: 161 |
| Kabat CDR-L2 | QMSNRAS | 197 | hu9F5VLv8_DIM29, SEQ ID NO: 160 |
| Kabat CDR-L2 | QGSNGAS | 198 | hu9F5VLv8_DIM1, SEQ ID NO: 132, |
| | | | and |
| | | | hu9F5VLv9 DIML SEQ ID NO: 162 |
| Kabat CDR-L2 | QGSNTAS | 199 | hu9F5VLv8_DIM3, SEQ ID NO: 134, |
| | | | hu9F5VLv8_DIM14, SEQ ID NO: 145, |
| | | | hu9F5VLv8_DIM15, SEQ ID NO: 146, |
| | | | hu9F5VLv8_DIM16, SEQ ID NO: 147, |
| | | | and |
| | | | hu9F5VLv8_DIM17, SEQ ID NO: 148 |
| Kabat CDR-L2 | QKSNRAS | 200 | hu9F5VLv8_DTM7, SEQ ID NO: 138, |
| | | | hu9F5VLv8_DIM8, SEQ ID NO: 139, |
| | | | and |
| | | | hu9F5VLv9_DIM8, SEQ ID NO: 166 |
| Kabat CDR-L2 | QKSNGAS | 201 | hu9F5VLv8_DIM9, SEQ ID NO: 140, |
| | | | hu9F5VLv8_DIM10, SEQ ID NO: 141, |
| | | | and |
| | | | hu9F5VLv9_DIM10, SEQ ID NO: 167 |
| Kabat CDR-L2 | QISNRAS | 202 | hu9F5VLv8_DIM19, SEQ ID NO: 150, |
| | | | and |
| | | | hu9F5VLv9_DIM19, SEQ ID NO, 170 |
| Kabat CDR-L2 | QISNGAS | 203 | hu9F5VLv8_DIM20, SEQ ID NO: 151, |
| | | | hu9F5VLv8_DIM21, SEQ ID NO, 152, |
| | | | and |
| | | | hu9F5VLv9_D1M20, SEQ ID NO: 171 |
| Kabat CDR-L2 | QESNRAS | 204 | hu9F5VLv8_DIM22, SEQ ID NO: 153, |
| | | | hu9F5VLv8_DIM24, SEQ ID NO 155, |
| | | | hu9F5VLv8_D1M25, SEQ ID NO: 156, |
| | | | and |
| | | | hu9F5VLv8_DIM26, SEQ ID NO: 157 |
| Kabat CDR-L2 | QESNGAS | 205 | hu9F5VLv8_DIM23, SEQ ID NO: 154 |
| Kabat CDR-L3 | GQNLELPLT | 206 | hu9F5VLv2_A89G, SEQ ID NO: 108 |
| Kabat CDR-L3 | AQNDELPLT | 207 | hu9F5VLv2_L92D, SEQ ID NO: 100 |
| Kabat CDR-L3 | AQNEELPLT | 208 | hu9F5VLv2_L92E, SEQ ID NO: 102 |
| Kabat CDR-L3 | AQNGELPLT | 209 | hu9F5VLv2_L92G, SEQ ID NO: 103, |
| | | | hu9F5VLv8_D1M4, SEQ ID NO: 135, |
| | | | hu9F5VLv8_DIM12, SEQ ID NO: 143, |
| | | | hu9F5VLv8_DIM15, SEQ ID NO: 146, |
| | | | hu9F5VLv8_DIM25, SEQ ID NO: 156, |
| | | | and hu9F5VLv9_D1M4, SEQ ID |
| | | | NO: 164 |
| Kabat CDR-L3 | AQNQELPLT | 210 | hu9F5VLv2_L92Q, SEQ ID NO: 104 |
| Kabat CDR-L3 | AQNTELPLT | 211 | hu9F5VLv2_L92T, SEQ ID NO: 107 |
| Kabat CDR-L3 | AQNIELPLT | 212 | hu9F5VLv8_DIM1, SEQ ID NO: 132, |
| | | | hu9F5VLv8_DIM2, SEQ ID NO: 133, |
| | | | hu9F5VLv8_DIM3, SEQ ID NO: 134, |
| | | | hu9F5VLv8_DIM5, SEQ ID NO: 136, |
| | | | hu9FSVLv8_DIM6, SEQ ID NO: 137, |
| | | | hu9F5VLv8_DIM7, SEQ ID NO: 138, |
| | | | hu9F5VLv8_DIMS, SEQ ID NO: 139, |
| | | | hu9F5VLv8_DIM9, SEQ ID NO 140, |
| | | | hu9F5VLv8_DIM10, SEQ ID NO: 141, |
| | | | hu9F5VLv8_DIM11, SEQ ID NO 142, |
| | | | hu9F5VLv8_DLM14, SEQ ID NO: 145, |
| | | | hu9F5VLv8_DIM17, SEQ ID NO, 148, |
| | | | hu9F5VLv8_DIM18, SEQ ID NO: 149, |
| | | | hu9F5VLv8_DIM19, SEQ ID NO: 150, |
| | | | hu9F5VLv8_DIM20, SEQ ID NO 151, |
| | | | hu9F5VLv8_DIM22, SEQ ID NO: 153, |
| | | | hu9F5VLv8_DIM23, SEQ ID NO: 154, |
| | | | hu9F5VLv8_DIM24, SEQ ID NO: 155, |
| | | | hu9F5VLv8_DIM27, SEQ ID NO: 158, |
| | | | hu9F5VLv8_DIM28, SEQ ID NO: 159, |
| | | | hu9F5VLv8_DIM29, SEQ ID NO: 160, |
| | | | hu9F5VLv8_D1M30, SEQ ID NO: 161, |
| | | | hu9F5VLv9_DIM1, SEQ ID NO: 162, |
| | | | hu9F5VLv9_D1M2, SEQ ID NO: 163, |
| | | | hu9F5VLv9_DIM5, SEQ ID NO: 165, |
| | | | hu9F5VLv9_DIMS, SEQ ID NO: 166, |
| | | | hu9F5VLv9_DIM10, SEQ ID NO 167, |
| | | | hu9F5VLv9_DIM11, SEQ ID NO: 168, |
| | | | hu9F5VLv9_DIM19, SEQ ID NO, 170, |
| | | | and hu9F5VLv9_DIM20, SEQ ID |
| | | | NO: 171 |
| Kabat CDR-L3 | AQNLGLPLT | 213 | hu9F5VLv2_L93G, SEQ ID NO: 105 |
| Kabat CDR-H2 | WIDPENGDT | 220 | Mouse 12C4 VH |
| | AYASKFQG | | |
| Kabat-Chothia | AFNIKDDYMN | 226 | Mouse 17C12 VH |
| composite CDR-H1 | | | |
| Kabat CDR-H2 | WIDPENGDTKYASKFQG | 227 | Mouse 17C12 VH |
| Kabat CDR-Ll | TSSQSLLHSNRKTYLH | 229 | Mouse 17C12 VL |
| Kabat CDR-L2 | LVSKLES | 230 | Mouse I7C12 VLv |
| Kabat CDR-L3 | LQTTHFPRT | 231 | Mouse I7C12 VL |
| Kabat-Chothia | GFSLSTYGMGVG | 241 | Mouse 14H3 VH |
| composite CDR-H1 | | | |
| Kabat CDR-H2 | NIWWDDIKYYNAALKS | 242 | Mouse 14H3 VH |
| Kabat CDR-H3 | NVDY | 243 | Mouse 14H3 VH |
| Kabat CDR-L1 | RSSOSLVHSNGNTFLH | 245 | Mouse 14H3 VL |
| Kabat CDR-L2 | KVSNRFS | 246 | Mouse 14H3 VL |
| Kabat CDR-L3 | SQSTLVPWT | 247 | Mouse 14H3 VL |
| Contact CDR-H2 | WIGWIDPENGDTA | 258 | Mouse 12C4 VH |
| Chothia CDR-H1 | AFNIKDD | 259 | Mouse 17C12 VH |
| AbM CDR-H2 | WIDPENGDTK | 260 | Mouse 17C12 VH |
| Contact CDR-H2 | WIGWIDPENGDTK | 261 | Mouse 17C12 VH |
| Contact CDR-L1 | KTYLHWL | 262 | Mouse I7C12 VL |
| Contact CDR-L2 | LLIYLVSKLE | 263 | Mouse 17C12 VL |
| Contact CDR-L3 | LQTTHFPR | 264 | Mouse 17C12 VL |
| Kabat | TYGMGVG | 265 | Mouse 14H3 VH |
| CDR-H1 | | | |
| Chothia CDR-H1 | GFSLSTYGM | 266 | Mouse 14H3 VH |
| Chothia CDR-H2 | WWDDI | 267 | Mouse 14H3 VH |
| AbM CDR-H2 | NIWWDDIKY | 268 | Mouse 14H3 VH |
| Contact CDR-H1 | STYGMGVG | 269 | Mouse 14H3 VH |
| Contact CDR-H2 | WLANIWWDDIKY | 270 | Mouse 14H3 VH |
| Contact CDR-H3 | ARNVD | 271 | Mouse 14H3 VH |
| Contact CDR-L1 | NTFLHWY | 272 | Mouse 14H3 VL |
| Contact CDR-L2 | LLIYKVSNRF | 273 | Mouse 14H3 VLv |
| Contact CDR-L3 | SQSTLVPW | 274 | Mouse 14H3 VL |
| Kabat-Chothia | GFSLSTYGMGVS | 275 | hu14H3VHv1 and hu14H3VHv2 |
| composite CDR-H1 | | | |

Listing of Sequences
P10636-8 (SEQ ID NO: 1)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMVDSPQLATLADEVSASLAKQGL
P10636-7 (SEQ ID NO: 2)
MAEPRQEFEVMEDHAGTYGLGDRKDQGGYTMHQDQEGDTDAGLKESPLQT
PTEDGSEEPGSETSDAKSTPTAEAEEAGIGDTPSLEDEAAGHVTQARMVS
KSKDGTGSDDKKAKGADGKTKIATPRGAAPPGQKGQANATRIPAKTPPAP
KTPPSSGEPPKSGDRSGYSSPGSPGTPGSRSRTPSLPTPPTREPKKVAVV
RTPPKSPSSAKSRLQTAPVPMPDLKNVKSKIGSTENLKHQPGGGKVQIIN
KKLDLSNVQSKCGSKDNIKHVPGGGSVQIVYKPVDLSKVTSKCGSLGNIH
HKPGGGQVEVKSEKLDFKDRVQSKIGSLDNITHVPGGGNKKIETHKLTFR
ENAKAKTDHGAEIVYKSPVVSGDTSPRHLSNVSSTGSIDMVDSPQLATLA
DEVSASLAKQGL
P10636-6 (4RON human tau) (SEQ ID NO: 3)
MAEPRQEFEVMEDHAGTYGLGDRKDQGGYTMHQDQEGDTDAGLKAEEAGI
GDTPSLEDEAAGHVTQARMVSKSKDGTGSDDKKAKGADGKTKIATPRGAA
PPGQKGQANATRIPAKTPPAPKTPPSSGEPPKSGDRSGYSSPGSPGTPGS
RSRTPSLPTPPTREPKKVAVVRTPPKSPSSAKSRLQTAPVPMPDLKNVKS
KIGSTENLKHQPGGGKVQIINKKLDLSNVQSKCGSKDNIKHVPGGGSVQI
VYKPVDLSKVTSKCGSLGNIHHKPGGGQVEVKSEKLDFKDRVQSKIGSLD
NITHVPGGGNKKIETHKLTFRENAKAKTDHGAEIVYKSPVVSGDTSPRHL
SNVSSTGSIDMVDSPQLATLADEVSASLAKQGL
P10636-5 (SEQ ID NO: 4)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIVYKPVDLSKVTSKCGSLGNIHHK
PGGGQVEVKSEKLDFKDRVQSKIGSLDNITHVPGGGNKKIETHKLTFREN
AKAKTDHGAEIVYKSPVVSGDTSPRHLSNVSSTGSIDMVDSPQLATLADE
VSASLAKQGL
P10636-4 (SEQ ID NO: 5)
MAEPRQEFEVMEDHAGTYGLGDRKDQGGYTMHQDQEGDTDAGLKESPLQT
PTEDGSEEPGSETSDAKSTPTAEAEEAGIGDTPSLEDEAAGHVTQARMVS
KSKDGTGSDDKKAKGADGKTKIATPRGAAPPGQKGQANATRIPAKTPPAP
KTPPSSGEPPKSGDRSGYSSPGSPGTPGSRSRTPSLPTPPTREPKKVAVV
RTPPKSPSSAKSRLQTAPVPMPDLKNVKSKIGSTENLKHQPGGGKVQIVY
KPVDLSKVTSKCGSLGNIHHKPGGGQVEVKSEKLDFKDRVQSKIGSLDNI
THVPGGGNKKIETHKLTFRENAKAKTDHGAEIVYKSPVVSGDTSPRHLSN
VSSTGSIDMVDSPQLATLADEVSASLAKQGL
P10636-2 (SEQ ID NO: 6)
MAEPRQEFEVMEDHAGTYGLGDRKDQGGYTMHQDQEGDTDAGLKAEEAGI
GDTPSLEDEAAGHVTQARMVSKSKDGTGSDDKKAKGADGKTKIATPRGAA
PPGQKGQANATRIPAKTPPAPKTPPSSGEPPKSGDRSGYSSPGSPGTPGS
RSRTPSLPTPPTREPKKVAVVRTPPKSPSSAKSRLQTAPVPMPDLKNVKS
KIGSTENLKHQPGGGKVQIVYKPVDLSKVTSKCGSLGNIHHKPGGGQVEV
KSEKLDFKDRVQSKIGSLDNITHVPGGGNKKIETHKLTFRENAKAKTDHG
AEIVYKSPVVSGDTSPRHLSNVSSTGSIDMVDSPQLATLADEVSASLAKQ
GL
SEQ ID NO: 7:
Heavy chain variable region of mouse 9F5 antibody
>m9F5VH
EVQLQQSGAELVRPGASVKLSCTASGFNIKDDYMNWVKQRPERGLEWIGW
IDPENGDTEYASKFQGKATMTADTSSNTAYLQFSSLTSEDTAVYYCTTSN
GWGQGTLVTVST
SEQ ID NO: 8: Kabat/Chothia composite CDR-H1 of
mouse 9F5 antibody
GFNIKDDYMN
SEQ ID NO: 9: Kabat CDR-H2 of mouse 9F5 antibody
WIDPENGDTEYASKFQG
SEQ ID NO: 10: Kabat CDR-H3 of mouse 9F5 antibody
SNG
SEQ ID NO: 11: Light chain variable region of
mouse 9F5 antibody
>m9F5VL
DIVMTQAAFSNPVTLGTSASISCRSSKSLLHSNGITYLYWYLQKPGQSPQ
LLIYQMSNLASGVPDRFSSSGSGTDFTLRISRVEAEDVGVYYCAQNLELP
LTFGAGTKLELK
SEQ ID NO: 12: Kabat CDR-L1 of mouse 9F5 antibody
RSSKSLLHSNGITYLY
SEQ ID NO: 13: Kabat CDR-L2 of mouse 9F5 antibody
QMSNLAS
SEQ ID NO: 14: Kabat CDR-L3 of mouse 9F5 antibody
AQNLELPLT
SEQ ID NO: 15:
Heavy chain variable region hu9F5VHv1
QVQLQQSGAELVKPGASVKLSCTASGFNIKDDYMNWVKQRPEQGLEWIGW
IDPENGDTEYASKFQGKATITADTSSNTAYLQLSSLTSEDTAVYYCASSN
GWGQGTTLTVSS
SEQ ID NO: 16:
Heavy chain variable region hu9F5VHv2
EVQLQQSGAELVKPGATVKISCTASGFNIKDDYMNWVKQRPEQGLEWIGW
IDPENGDTEYASKFQGKATMTADTSTNTAYLQLSSLTSEDTAVYYCTTSN
GWGQGTTVTVSS
SEQ ID NO: 17:
Heavy chain variable region hu9F5VHv3
EVQLQQSGAELVKPGATVKISCTASGFNIKDDYMNWVKQRPEQGLEWIGW
IDPENGDTEYASKFQGRATMTADTSTNTAYLELSSLTSEDTAVYYCTTSN
GWGQGTTVTVSS
SEQ ID NO: 18:
Heavy chain variable region hu9F5VHv4
EVQLQQSGAELVKPGATVKISCKASGFTIKDDYMNWVKQRPEQGLEWIGW
IDPENGDTEYASKFQGRATMTADTSTNTAYLELSSLRSEDTAVYYCTTSN
GWGQGTTVTVSS
SEQ ID NO: 19:
Heavy chain variable region hu9F5VHv5
EVQLQQSGAELVKPGATVKISCKASGFTIKDDYMNWVKQRPEKGLEWIGW
VDPEDGETEYASKFQGRATMTADTSTDTAYMELSSLRSEDTAVYYCTTSN
GWGQGTLVTVSS
SEQ ID NO: 20:
Heavy chain variable region hu9F5VHv6
EVQLQQSGAELVKPGATVKISCKASGFTIKDDYMNWVKQAPEKGLEWMGW
VDPEDGETEYASKFQGRATMTADTSTDTAYMELSSLRSEDTAVYYCTTSN
GWGQGTLVTVSS
SEQ ID NO: 21:
Heavy chain variable region hu9F5VHv7
EVQLVQSGAEVKKPGATVKISCKASGFNIKDDYMNWVRQAPGKGLEWIGW
VDPEDGETEYASKFQGRATMTADTSTDTAYMELSSLRSEDTAVYYCTTSN
GWGQGTLVTVSS
SEQ ID NO: 22:
Heavy chain variable region hu9F5VHv8
EVQLVQSGAEVKKPGATVKISCKASGFNIKDDYMNWVRQAPGKGLEWIGW
VDPENGDTEYASKFQGRATMTADTSTDTAYMELSSLRSEDTAVYYCTTSN
GWGQGTLVTVSS
SEQ ID NO: 23:
Light chain variable region hu9F5VLv1
DIVMTQAAFSNPVTLGTSASISCRSSKSLLHSNGITYLYWYLQRPGQSPQ
LLIYQMSNLASGVPNRFSSSGSGTDFTLRISRVEAEDVGVYYCAQNLELP
LTFGGGTKLEIK
SEQ ID NO: 24:
Light chain variable region hu9F5VLv2
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 25:
Light chain variable region hu9F5VLv3
DIVMTQSPFSNPVTPGESASISCRSSKSLLHSNGITYLYWYLQRPGQSPQ
LLIYQMSNLASGVPNRFSGSGSGTDFTLRISRVEAEDVGVYYCAQNLELP
LTFGQGTKLEIK
SEQ ID NO: 26:
Light chain variable region hu9F5VLv4
DIVMTQSPFSLPVTPGESASISCRSSKSLLHSNGITYLYWYLQRPGQSPQ
LLIYQGSNRASGVPNRFSGSGSGTDFTLRISRVEAEDVGVYYCAQNLELP
LTFGQGTKLEIK
SEQ ID NO: 27:
Light chain variable region hu9F5VLv5
DIVMTQSPFSLPVTPGESASISCRSSKSLLHSNGYTYLYWYLQRPGQSPQ
LLIYQGSNRASGVPNRFSGSGSGTDFTLRISRVEAEDVGVYYCAQNLELP
LTFGQGTKLEIK
SEQ ID NO: 28:
Light chain variable region hu9F5VLv6
DIVMTQSPFSLPVTPGESASISCRSSKSLLHSNGYTYLYWYLQRPGQSPQ
LLIYQGSNRASGVPNRFSGSESGTDFTLRISRVEAEDVGVYYCAQNLELP
LTFGQGTKLEIK
SEQ ID NO: 29:
Light chain variable region hu9F5VLv7
DIVMTQSPLSLPVTPGEPASISCRSSKSLLHSNGINYLYWYLQKPGQSPQ
LLIYQGSNRASGVPDRFSGSGSGTDFTLKISRVEAEDVGVYYCAQNLELP
LTFGQGTKLEIK
SEQ ID NO: 30:
Heavy chain variable region structural model
PDB.# 50BF-VH_mSt
EVQLQQSGAELVEPGASVKLSCTGSGFNIKVYYVHWLKQLTEQGLEWIGR
IDPENGETIYTPKFQDKATLTVDTSSNTAYLQLSSLTSEDAAVYYCVSSG
YWGQGTTLTVSS
SEQ ID NO: 31:
Heavy chain variable region acceptor GenBank
Acc.# AAN16432-VH_huFrwk
EVQLVQSGAEVKKPGASVKVSCKVSGYTLTELSMIHWVRQAPGKGLEWMG
GFDPEDGETIYAQKFQGRVTMTEDTSTDTAYMELSSLRSEDTAVYYCAGY
RSPMPTANKWGQGTLVTVSS
SEQ ID NO: 32:
Heavy chain variable region acceptor PDB +190
2RCS-VH_huFrwk
QVQLQQSGAELVKPGASVKLSCTASGFNIKDTYMIHWVKQRPEQGLEWIG
RIDPANGNTKYDPKFQGKATITADTSSNTAYLQLSSLTSEDTAVYYCASY
YGIYWGQGTTLTVSS
SEQ ID NO: 33:
Heavy chain variable region germline sequence
IMGT# IGHV1-69-2*01
EVQLVQSGAEVKKPGATVKISCKVSGYTFTDYYMHWVQQAPGKGLEWMGL
VDPEDGETIYAEKFQGRVTITADTSTDTAYMELSSLRSEDTAVYYCAT-
Q--HWGQGTLVTVSS
SEQ ID NO: 34:
Light chain variable region structural model
PDB # 50BF-VL_mSt
DIVMTQSAFSNPVTLGTSASISCRSSKSLLHRNGITYLYWYLQKPGQPPQ
LLIYQMSNLASGVPDRFTSSGSGTDFTLKISRVEAEDVGVYYCAQNLELW
TFGGGTKLEIK
SEQ ID NO: 35:
Light chain variable region acceptor GenBank
Acc. # CAB51297-VL_huFrwk
DIVMTQSPLSLPVTPGEPASISCRSSQSLLHSNGYNYLDWYLQKPGQSPQ
LLIYLGSNRASGVPDRFSGSGSGTDFTLKISRVEAEDVGVYYCMQALQTP
LTFGGGTKVEIK
SEQ ID NO: 36:
Light chain variable region acceptor GenBank
Acc. # 1911357B- VL_huFrwk
DIVMTQAAFSNPVTLGTSASISCRSSKNLLHSNGITFLYWYLQRPGQSPQ
LLIYRVSNLASGVPNRFSGSESGTDFTLRISRVEAEDVGVYYCAQLLELP
YTFGGGTKLEIK
SEQ ID NO: 37:
Light chain variable region germline sequence
EVIGT# IGKV2-28*01 & IGKJ2 *01
DIVMTQSPLSLPVTPGEPASISCRSSQSLLHSNGYNYLDWYLQKPGQSPQ
LLIYLGSNRASGVPDRFSGSGSGTDFTLKISRVEAEDVGVYYCMQALQTP
YTFGQGTKLEIK
SEQ ID NO: 38:
Nucleic acid sequence encoding heavy chain
variable region of mouse 9F5 antibody
ATGAAATGCAGCTGGGTCATCTTCTTCCTGATGGCAGTGGTTATAGGGGT
CAATTCAGAGGTTCAGCTGCAGCAGTCTGGGGCTGAACTTGTGAGGCCAG
GGGCCTCAGTCAAGTTGTCCTGCACAGCTTCTGGCTTTAACATTAAAGAC
GACTATATGAACTGGGTGAAACAGAGACCTGAACGGGGCCTGGAGTGGAT
TGGATGGATTGATCCTGAGAATGGTGATACTGAATATGCCTCGAAGTTCC
AGGGAAAGGCCACTATGACTGCAGACACATCCTCCAACACAGCCTACCTG
CAGTTCAGCAGCCTGACATCTGAGGACACTGCCGTCTATTACTGTACTAC
AAGTAACGGCTGGGGCCAAGGGACTCTGGTCACTGTCTCTACA
SEQ ID NO: 39:
Nucleic acid sequence encoding light chain
variable region of mouse 9F5 antibody
ATGAGGTTCTCTGCTCAGCTTCTGGGGCTGCTTGTGCTCTGGATCCCTGG
ATCCATTGCAGATATTGTGATGACGCAGGCTGCATTCTCCAATCCAGTCA
CTCTTGGAACATCAGCTTCCATCTCCTGCAGGTCTAGTAAGAGTCTCCTA
CATAGTAATGGCATCACTTATTTGTATTGGTATCTGCAGAAGCCAGGCCA
GTCTCCTCAGCTCCTGATTTATCAGATGTCCAACCTTGCCTCAGGAGTCC
CAGACAGGTTCAGTAGCAGTGGGTCAGGAACTGATTTCACACTGAGAATC
AGCAGAGTGGAGGCTGAGGATGTGGGTGTTTATTACTGTGGTCAAAATCT
AGAACTTCCGCTCACGTTCGGTGCTGGGACCAAGCTGGAGCTGAAA
SEQ ID NO: 40: Kabat CDR-H1 of mouse 9F5 antibody
DDYMN
SEQ ID NO: 41: Chothia CDR-H1 of mouse 9F5 antibody
GFNIKDD
SEQ ID NO: 42: Chothia CDR-H2 of mouse 9F5 antibody
DPENGD
SEQ ID NO: 43: AbM CDR-H2 of mouse 9F5 antibody
WIDPENGDTE
SEQ ID NO: 44: Contact CDR-H1 of mouse 9F5 antibody
KDDYMN
SEQ ID NO: 45: Contact CDR-H2 of mouse 9F5 antibody
WIGWIDPENGDTE
SEQ ID NO: 46: Contact CDR-H3 of mouse 9F5 antibody
TTSN
SEQ ID NO: 47: Contact CDR-L1 of mouse 9F5 antibody
ITYLYWY
SEQ ID NO: 48: Contact CDR-L2 of mouse 9F5 antibody
LLIYQMSNLA
SEQ ID NO: 49: Contact CDR-L3 of mouse 9F5 antibody
AQNLELPL
SEQ ID NO: 50:
Alternate Kabat-Chothia Composite CDR-H1
of a humanized 9F5 antibody (present in
hu9F5VHv4, hu9F5VHv5, and hu9F5VHv6)
GFTIKDDYMN
SEQ ID NO: 51:
Alternate Kabat CDR-H2 of a humanized
9F5 antibody (present in
hu9F5VHv5, hu9F5VHv6, and hu9F5VHv7)
WVDPEDGETEYASKFQG
SEQ ID NO: 52: Alternate Kabat CDR-H2 of a
humanized 9F5 antibody (present in
hu9F5VHv8)
WVDPENGDTEYASKFQG
SEQ ID NO: 53:
Alternate Kabat CDR-L1 of a humanized 9F5
antibody (present in hu9F5VLv5 and hu9F5VLv6)
RSSKSLLHSNGYTYLY
SEQ ID NO: 54:
Alternate Kabat CDR-L1 of a humanized 9F5
antibody (present in hu9F5VLv7)
RSSKSLLHSNGINYLY
SEQ ID NO: 55: Alternate Kabat CDR-L2 of a
humanized 9F5 antibody (present in hu9F5VLv4,
hu9F5VLv5, hu9F5VLv6, hu9F5VLv7, SEQ ID NO:
133, 135, 136, 137, 142, 143, 144, 149, 158,
159, 163, 164, 165, 168, 169))
QGSNRAS
SEQ ID NO: 56: Epitope of antibody 9F5
(Q/E)IVYK(S/P)
SEQ ID NO: 57:
Consensus motif of a peptide bound by
antibody 9F5, 10C12, 2D11, and 17C12
QIVYKP
SEQ ID NO: 58: Consensus motif of a peptide
bound by antibody 9F5, 10C12, and 17C12
EIVYKSP
SEQ ID NO: 59: Linker
GSGSGSG
SEQ ID NO: 60: HA control peptide
YPYDVPDYAG
SEQ ID NO: 61:
Variant of hu9F5VLv2 light chain variable
region (also known as hu9F5VLv2_M51T)
DIVMTQSPFSNPVTPGTSASISCRSSKSLLHSNGITYLYWYLQRPGQSPQ
LLIYQESNLASGVPNRFSSSGSGTDFTLRISRVEAEDVGVYYCAQNLELP
LTFGQGTKLEIK
SEQ ID NO: 62: Variant of hu9F5VLv2 light chain
variable region (also known as
hu9F5VLv2_M51D)
DIVMTQSPFSNPVTPGTSASISCRSSKSLLHSNGITYLYWYLQRPGQSPQ
LLIYQDSNLASGVPNRFSSSGSGTDFTLRISRVEAEDVGVYYCAQNLELP
LTFGQGTKLEIK
SEQ ID NO: 63: Variant of hu9F5VLv2 light chain
variable region (also known as
hu9F5VLv2_L27cD)
DIVMTQSPFSNPVTPGTSASISCRSSKSLDHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 64: Variant of hu9F5VLv2_Light chain
variable region (also known as
hu9F5VLv2_L27cG)
DIVMTQSPFSNPVTPGTSASISCRSSKSLGHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 65: Variant of hu9F5VLv2_Light chain
variable region (also known as
hu9F5VLv2_L27cS)
DIVMTQSPFSNPVTPGTSASISCRSSKSLSHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 66: Variant of hu9F5VLv2_Light chain
variable region (also known as
hu9F5VLv2_L27cE)
DIVMTQSPFSNPVTPGTSASISCRSSKSLEHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 67: Variant of hu9F5VLv2_Light chain
variable region (also known as
hu9F5VLv2_I30E)
DIVMTQSPFSNPVTPGTSASISCRSSKSLLHSNGETYLYWYLQRPGQSPQ
LLIYQMSNLASGVPNRFSSSGSGTDFTLRISRVEAEDVGVYYCAQNLELP
LTFGQGTKLEIK
SEQ ID NO: 68: Variant of hu9F5VLv2_Light chain
variable region (also known as
hu9F5VLv2_I30K)
DIVMTQSPFSNPVTPGTSASISCRSSKSLLHSNGKTYLYWYLQRPGQSPQ
LLIYQMSNLASGVPNRFSSSGSGTDFTLRISRVEAEDVGVYYCAQNLELP
LTFGQGTKLEIK
SEQ ID NO: 69: Variant of hu9F5VLv2_Light chain
variable region (also known as
hu9F5VLv2_L27cT)
DIVMTQSPFSNPVTPGTSASISCRSSKSLTHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 70: Variant of hu9F5VLv2_Light chain
variable region (also known as
hu9F5VLv2_L27cN)
DIVMTQSPFSNPVTPGTSASISCRSSKSLNHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 71: Variant of hu9F5VLv2_Light chain
variable region (also known as
hu9F5VLv2_L27bD)
DIVMTQSPFSNPVTPGTSASISCRSSKSDLHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 72: Variant of hu9F5VLv2_Light chain
variable region (also known as
hu9F5VLv2 130G)
DIVMTQSPFSNPVTPGTSASISCRSSKSLLHSNGGTYLYWYLQRPGQSPQ
LLIYQMSNLASGVPNRFSSSGSGTDFTLRISRVEAEDVGVYYCAQNLELP
LTFGQGTKLEIK
SEQ ID NO: 73: Variant of hu9F5VLv2_Light chain
variable region (also known as
hu9F5VLv2_L33N)
DIVMTQSPFSNPVTPGTSASISCRSSKSLLHSNGITYNYWYLQRPGQSPQ
LLIYQMSNLASGVPNRFSSSGSGTDFTLRISRVEAEDVGVYYCAQNLELP
LTFGQGTKLEIK
SEQ ID NO: 74: Variant of hu9F5VLv2_Light chain
variable region (also known as
hu9F5VLv2_L27cA)
DIVMTQSPFSNPVTPGTSASISCRSSKSLAHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 75: Variant of hu9F5VLv2_Light chain
variable region (also known as
hu9F5VLv2_L33T)
DIVMTQSPFSNPVTPGTSASISCRSSKSLLHSNGITYTYWYLQRPGQSPQ
LLIYQMSNLASGVPNRFSSSGSGTDFTLRISRVEAEDVGVYYCAQNLELP
LTFGQGTKLEIK
SEQ ID NO: 76: Variant of hu9F5VLv2_Light chain
variable region (also known as
hu9F5VLv2_L33 S)
DIVMTQSPFSNPVTPGTSASISCRSSKSLLHSNGITYSYWYLQRPGQSPQ
LLIYQMSNLASGVPNRFSSSGSGTDFTLRISRVEAEDVGVYYCAQNLELP
LTFGQGTKLEIK
SEQ ID NO: 77: Variant of hu9F5VLv2_Light chain
variable region (also known as
hu9F5VLv2_L33R)
DIVMTQSPFSNPVTPGTSASISCRSSKSLLHSNGITYRYWYLQRPGQSPQ
LLIYQMSNLASGVPNRFSSSGSGTDFTLRISRVEAEDVGVYYCAQNLELP
LTFGQGTKLEIK
SEQ ID NO: 78: Variant of hu9F5VLv2_Light chain
variable region (also known as
hu9F5VLv2_I30Q)
DIVMTQSPFSNPVTPGTSASISCRSSKSLLHSNGQTYLYWYLQRPGQSPQ
LLIYQMSNLASGVPNRFSSSGSGTDFTLRISRVEAEDVGVYYCAQNLELP
LTFGQGTKLEIK
SEQ ID NO: 79: Variant of hu9F5VLv2_Light chain
variable region (also known as
hu9F5VLv2_L27bT)
DIVMTQSPFSNPVTPGTSASISCRSSKSTLHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 80: Variant of hu9F5VLv2_Light chain
variable region (also known as
hu9F5VLv2 SEQ ID NO: 146)
DIVMTQSPFSNPVTPGTSASISCRSSKSLLHSNGIGYLYWYLQRPGQSPQ
LLIYQMSNLASGVPNRFSSSGSGTDFTLRISRVEAEDVGVYYCAQNLELP
LTFGQGTKLEIK
SEQ ID NO: 81: Variant of hu9F5VLv2_Light chain
variable region (also known as
hu9F5VLv2_L27bQ)
DIVMTQSPFSNPVTPGTSASISCRSSKSQLHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 82: Variant of hu9F5VLv2_Light chain
variable region (also known as
hu9F5VLv2_L33G)
DIVMTQSPFSNPVTPGTSASISCRSSKSLLHSNGITYGYWYLQRPGQSPQ
LLIYQMSNLASGVPNRFSSSGSGTDFTLRISRVEAEDVGVYYCAQNLELP
LTFGQGTKLEIK
SEQ ID NO: 83: Variant of hu9F5VLv2_Light chain
variable region (also known as
hu9F5VLv2_L27cP)
DIVMTQSPFSNPVTPGTSASISCRSSKSLPHSNGITYLYWYLQRPGQSPQ
LLIYQMSNLASGVPNRFSSSGSGTDFTLRISRVEAEDVGVYYCAQNLELP
LTFGQGTKLEIK
SEQ ID NO: 84: Variant of hu9F5VLv2_Light chain
variable region (also known as
hu9F5VLv2 V78R)
DIVMTQSPFSNPVTPGTSASISCRSSKSLLHSNGITYLYWYLQRPGQSPQ
LLIYQMSNLASGVPNRFSSSGSGTDFTLRISRREAEDVGVYYCAQNLELP
LTFGQGTKLEIK
SEQ ID NO: 85: Variant of hu9F5VLv2_Light chain
variable region (also known as
hu9F5VLv2_I75D)
DIVMTQSPFSNPVTPGTSASISCRSSKSLLHSNGITYLYWYLQRPGQSP
QLLIYQMSNLASGVPNRFSSSGSGTDFTLRDSRVEAEDVGVYYCAQNLE
LPLTFGQGTKLEIK
SEQ ID NO: 86: Variant of hu9F5VLv2_Light chain
variable region (also known as
hu9F5VLv2_V78D)
DIVMTQSPFSNPVTPGTSASISCRSSKSLLHSNGITYLYWYLQRPGQSP
QLLIYQMSNLASGVPNRFSSSGSGTDFTLRISRDEAEDVGVYYCAQNLE
LPLTFGQGTKLEIK
SEQ ID NO: 87: Variant of hu9F5VLv2_Light chain
variable region (also known as
hu9F5VLv2_V78E)
DIVMTQSPFSNPVTPGTSASISCRSSKSLLHSNGITYLYWYLQRPGQSP
QLLIYQMSNLASGVPNRFSSSGSGTDFTLRISREEAEDVGVYYCAQNLE
LPLTFGQGTKLEIK
SEQ ID NO: 88: Variant of hu9F5VLv2_Light chain
variable region (also known as
hu9F5VLv2_V78P)
DIVMTQSPFSNPVTPGTSASISCRSSKSLLHSNGITYLYWYLQRPGQSP
QLLIYQMSNLASGVPNRFSSSGSGTDFTLRISRPEAEDVGVYYCAQNLE
LPLTFGQGTKLEIK
SEQ ID NO: 89: Variant of hu9F5VLv2_Light chain
variable region (also known as
hu9F5VLv2_V78K)
DIVMTQSPFSNPVTPGTSASISCRSSKSLLHSNGITYLYWYLQRPGQS
PQLLIYQMSNLASGVPNRFSSSGSGTDFTLRISRKEAEDVGVYYCAQN
LELPLTFGQGTKLEIK
SEQ ID NO: 90: Variant of hu9F5VLv2_Light chain
variable region (also known as
hu9F5VLv2_R77D)
DIVMTQSPFSNPVTPGTSASISCRSSKSLLHSNGITYLYWYLQRPGQSPQ
LLIYQMSNLASGVPNRFSSSGSGTDFTLRISDVEAEDVGVYYCAQNLELP
LTFGQGTKLEIK
SEQ ID NO: 91: Variant of hu9F5VLv2_Light chain
variable region (also known as
hu9F5VLv2_V78G)
DIVMTQSPFSNPVTPGTSASISCRSSKSLLHSNGITYLYWYLQRPGQSPQ
LLIYQMSNLASGVPNRFSSSGSGTDFTLRISRGEAEDVGVYYCAQNLELP
LTFGQGTKLEIK
SEQ ID NO: 92: Variant of hu9F5VLv2_Light chain
variable region (also known as
hu9F5VLv2_S76P)
DIVMTQSPFSNPVTPGTSASISCRSSKSLLHSNGITYLYWYLQRPGQSPQ
LLIYQMSNLASGVPNRFSSSGSGTDFTLRIPRVEAEDVGVYYCAQNLELP
LTFGQGTKLEIK
SEQ ID NO: 93: Variant of hu9F5VLv2_Light chain
variable region (also known as
hu9F5VLv2_I75P)
DIVMTQSPFSNPVTPGTSASISCRSSKSLLHSNGITYLYWYLQRPGQSPQ
LLIYQMSNLASGVPNRFSSSGSGTDFTLRPSRVEAEDVGVYYCAQNLELP
LTFGQGTKLEIK
SEQ ID NO: 94: Variant of hu9F5VLv2_Light chain
variable region (also known as
hu9F5VLv2_I75Q)
DIVMTQSPFSNPVTPGTSASISCRSSKSLLHSNGITYLYWYLQRPGQSPQ
LLIYQMSNLASGVPNRFSSSGSGTDFTLRQSRVEAEDVGVYYCAQNLELP
LTFGQGTKLEIK
SEQ ID NO: 95: Variant of hu9F5VLv2_Light chain
variable region (also known as
hu9F5VLv2_I75G)
DIVMTQSPFSNPVTPGTSASISCRSSKSLLHSNGITYLYWYLQRPGQSPQ
LLIYQMSNLASGVPNRFSSSGSGTDFTLRGSRVEAEDVGVYYCAQNLELP
LTFGQGTKLEIK
SEQ ID NO: 96: Variant of hu9F5VLv2_Light chain
variable region (also known as
hu9F5VLv2_L73P)
DIVMTQSPFSNPVTPGTSASISCRSSKSLLHSNGITYLYWYLQRPGQSPQ
LLIYQMSNLASGVPNRFSSSGSGTDFTPRISRVEAEDVGVYYCAQNLELP
LTFGQGTKLEIK
SEQ ID NO: 97: Variant of hu9F5VLv2_Light chain
variable region (also known as
hu9F5VLv2_L73G)
DIVMTQSPFSNPVTPGTSASISCRSSKSLLHSNGITYLYWYLQRPGQSPQ
LLIYQMSNLASGVPNRFSSSGSGTDFTGRISRVEAEDVGVYYCAQNLELP
LTFGQGTKLEIK
SEQ ID NO: 98: Variant of hu9F5VLv2_Light chain
variable region (also known as
hu9F5VLv2 V78Q)
DIVMTQSPFSNPVTPGTSASISCRSSKSLLHSNGITYLYWYLQRPGQSPQ
LLIYQMSNLASGVPNRFSSSGSGTDFTLRISRQEAEDVGVYYCAQNLELP
LTFGQGTKLEIK
SEQ ID NO: 99: Variant of hu9F5VLv2_Light chain
variable region (also known as
hu9F5VLv2 S76G)
DIVMTQSPFSNPVTPGTSASISCRSSKSLLHSNGITYLYWYLQRPGQSPQ
LLIYQMSNLASGVPNRFSSSGSGTDFTLRIGRVEAEDVGVYYCAQNLELP
LTFGQGTKLEIK
SEQ ID NO: 100: Variant of hu9F5VLv2_Light chain
variable region (also known as
hu9F5VLv2_L92D)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNDELP
LTFGQGTKLEIK
SEQ ID NO: 101: Variant of hu9F5VLv2_Light chain
variable region (also known as
hu9F5VLv2 Y86T)
DIVMTQSPFSNPVTPGTSASISCRSSKSLLHSNGITYLYWYLQRPGQSPQ
LLIYQMSNLASGVPNRFSSSGSGTDFTLRISRVEAEDVGVTYCAQNLELP
LTFGQGTKLEIK
SEQ ID NO: 102: Variant of hu9F5VLv2_Light chain
variable region (also known as
hu9F5VLv2_L92E)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNEELP
LTFGQGTKLEIK
SEQ ID NO: 103: Variant of hu9F5VLv2_Light chain
variable region (also known as
hu9F5VLv2_L92G)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNGELP
LTFGQGTKLEIK
SEQ ID NO: 104: Variant of hu9F5VLv2_Light chain
variable region (also known as
hu9F5VLv2_L92Q)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNQELP
LTFGQGTKLEIK
SEQ ID NO: 105: Variant of hu9F5VLv2_Light chain
variable region (also known as
hu9F5VLv2_L93G)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNLEGP
LTFGQGTKLEIK
SEQ ID NO: 106: Variant of hu9F5VLv2_Light chain
variable region (also known as
hu9F5VLv2 V85G)
DIVMTQSPFSNPVTPGTSASISCRSSKSLLHSNGITYLYWYLQRPGQSPQ
LLIYQMSNLASGVPNRFSSSGSGTDFTLRISRVEAEDVGGYYCAQNLELP
LTFGQGTKLEIK
SEQ ID NO: 107: Variant of hu9F5VLv2_Light chain
variable region (also known as
hu9F5VLv2_L92T)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCAQNTELP
LTFGQGTKLEIK
SEQ ID NO: 108: Variant of hu9F5VLv2_Light chain
variable region (also known as
hu9F5VLv2 A89G)
DIVMTQSPFSNPVTPGTSASISCRSSKSLLHSNGITYLYWYLQRPGQSPQ
LLIYQMSNLASGVPNRFSSSGSGTDFTLRISRVEAEDVGVYYCGQNLELP
LTFGQGTKLEIK
SEQ ID NO: 109: Variant of hu9F5VHv4 heavy chain
variable region (also known as
hu9F5VHv4_L80P)
EVQLQQSGAELVKPGATVKISCKASGFTIKDDYMNWVKQRPEQGLEWIGW
IDPENGDTEYASKFQGRATMTADTSTNTAYPELSSLRSEDTAVYYCTTSN
GWGQGTTVTVSS
SEQ ID NO: 110: Variant of hu9F5VHv4 heavy chain
variable region (also known as
hu9F5VHv4_L80D)
EVQLQQSGAELVKPGATVKISCKASGFTIKDDYMNWVKQRPEQGLEWIGW
IDPENGDTEYASKFQGRATMTADTSTNTAYDELSSLRSEDTAVYYCTTSN
GWGQGTTVTVSS
SEQ ID NO: 111: Variant of hu9F5VHv4 heavy chain
variable region (also known as
hu9F5VHv4_L82cG)
EVQLQQSGAELVKPGATVKISCKASGFTIKDDYMNWVKQRPEQGLEWIGW
IDPENGDTEYASKFQGRATMTADTSTNTAYLELSSGRSEDTAVYYCTTSN
GWGQGTTVTVSS
SEQ ID NO: 112: Variant of hu9F5VHv4 heavy chain
variable region (also known as
hu9F5VHv4_L82cD)
EVQLQQSGAELVKPGATVKISCKASGFTIKDDYMNWVKQRPEQGLEWIGW
IDPENGDTEYASKFQGRATMTADTSTNTAYLELSSDRSEDTAVYYCTTSN
GWGQGTTVTVSS
SEQ ID NO: 113: Variant of hu9F5VHv4 heavy chain
variable region (also known as
hu9F5VHv4_L82P)
EVQLQQSGAELVKPGATVKISCKASGFTIKDDYMNWVKQRPEQGLEWIGW
IDPENGDTEYASKFQGRATMTADTSTNTAYLEPSSLRSEDTAVYYCTTSN
GWGQGTTVTVSS
SEQ ID NO: 114: Variant of hu9F5VHv4 heavy chain
variable region (also known as
hu9F5VHv4_L80G)
EVQLQQSGAELVKPGATVKISCKASGFTIKDDYMNWVKQRPEQGLEWIGW
IDPENGDTEYASKFQGRATMTADTSTNTAYGELSSLRSEDTAVYYCTTSN
GWGQGTTVTVSS
SEQ ID NO: 115: Variant of hu9F5VHv4 heavy chain
variable region (also known as
hu9F5VHv4_L82K)
EVQLQQSGAELVKPGATVKISCKASGFTIKDDYMNWVKQRPEQGLEWIGW
IDPENGDTEYASKFQGRATMTADTSTNTAYLEKSSLRSEDTAVYYCTTSN
GWGQGTTVTVSS
SEQ ID NO: 116: Variant of hu9F5VHv4 heavy chain
variable region (also known as
hu9F5VHv4_L82R)
EVQLQQSGAELVKPGATVKISCKASGFTIKDDYMNWVKQRPEQGLEWIGW
IDPENGDTEYASKFQGRATMTADTSTNTAYLERSSLRSEDTAVYYCTTSN
GWGQGTTVTVSS
SEQ ID NO: 117: Variant of hu9F5VHv4 heavy chain
variable region (also known as
hu9F5VHv4_L82E)
EVQLQQSGAELVKPGATVKISCKASGFTIKDDYMNWVKQRPEQGLEWIGW
IDPENGDTEYASKFQGRATMTADTSTNTAYLEESSLRSEDTAVYYCTTSN
GWGQGTTVTVSS
SEQ ID NO: 118: Variant of hu9F5VHv4 heavy chain
variable region (also known as
hu9F5VHv4_L82N)
EVQLQQSGAELVKPGATVKISCKASGFTIKDDYMNWVKQRPEQGLEWIGW
IDPENGDTEYASKFQGRATMTADTSTNTAYLENSSLRSEDTAVYYCTTSN
GWGQGTTVTVSS
SEQ ID NO: 119: Variant of hu9F5VHv4 heavy chain
variable region (also known as
hu9F5VHv4_Y79D)
EVQLQQSGAELVKPGATVKISCKASGFTIKDDYMNWVKQRPEQGLEWIGW
IDPENGDTEYASKFQGRATMTADTSTNTADLELSSLRSEDTAVYYCTTSN
GWGQGTTVTVSS
SEQ ID NO: 120: Variant of hu9F5VHv4 heavy chain
variable region (also known as
hu9F5VHv4_Y79N)
EVQLQQSGAELVKPGATVKISCKASGFTIKDDYMNWVKQRPEQGLEWIGW
IDPENGDTEYASKFQGRATMTADTSTNTANLELSSLRSEDTAVYYCTTSN
GWGQGTTVTVSS
SEQ ID NO: 121: Variant of hu9F5VHv4 heavy chain
variable region (also known as
hu9F5VHv4_Y79G)
EVQLQQSGAELVKPGATVKISCKASGFTIKDDYMNWVKQRPEQGLEWIGW
IDPENGDTEYASKFQGRATMTADTSTNTAGLELSSLRSEDTAVYYCTTSN
GWGQGTTVTVSS
SEQ ID NO: 122: Variant of hu9F5VHv5 heavy chain
variable region (also known as
hu9F5VHv5 M80E)
EVQLQQSGAELVKPGATVKISCKASGFTIKDDYMNWVKQRPEKGLEWIGW
VDPEDGETEYASKFQGRATMTADTSTDTAYEELSSLRSEDTAVYYCTTSN
GWGQGTLVTVSS
SEQ ID NO: 123: Variant of hu9F5VHv4 heavy chain
variable region (also known as
hu9F5VHv5 M80G)
EVQLQQSGAELVKPGATVKISCKASGFTIKDDYMNWVKQRPEKGLEWIGW
VDPEDGETEYASKFQGRATMTADTSTDTAYGELSSLRSEDTAVYYCTTSN
GWGQGTLVTVSS
SEQ ID NO: 124: Variant of hu9F5VHv4 heavy chain
variable region (also known as
hu9F5VHv4_L82cS)
EVQLQQSGAELVKPGATVKISCKASGFTIKDDYMNWVKQRPEQGLEWIGW
IDPENGDTEYASKFQGRATMTADTSTNTAYLELSSSRSEDTAVYYCTTSN
GWGQGTTVTVSS
SEQ ID NO: 125: Variant of hu9F5VHv4 heavy chain
variable region (also known as
hu9F5VHv4_Y79Q)
EVQLQQSGAELVKPGATVKISCKASGFTIKDDYMNWVKQRPEQGLEWIGW
IDPENGDTEYASKFQGRATMTADTSTNTAQLELSSLRSEDTAVYYCTTSN
GWGQGTTVTVSS
SEQ ID NO: 126: Variant of hu9F5VHv4 heavy chain
variable region (also known as
hu9F5VHv4 S82aG)
EVQLQQSGAELVKPGATVKISCKASGFTIKDDYMNWVKQRPEQGLEWIGW
IDPENGDTEYASKFQGRATMTADTSTNTAYLELGSLRSEDTAVYYCTTSN
GWGQGTTVTVSS
SEQ ID NO: 127: Heavy chain
variable region hu9F5VHv9
EVQLVQSGAEVICKPGATVKISCKASGENIKDDYMNWVQQRPGKGLETNI
GWIDPENGDTEYASKFQGRATMTADTSINTAYMELSSLRSEDTAVYYQTT
SNGWGQGTLVTVSS
SEQ ID NO: 128: Heavy chain
variable region hu9F5VHv10 (also known as
hu9F5VHv9_Q3_8K_G42E)
EVQLVQSGAEVKKPGATVKISCKASGFNIKDDYMNWVKQRPEKGLEWIGW
IDPENGDTEYASKFQGRATMTADTSTNTAYMELSSLRSEDTAVYYCTTSN
GWGQGTLVTVSS
SEQ ID NO: 129: Heavy chain
variable region hu9F5VHv10_L82cG
EVQLVQSGAEVKKPGATVKISCKASGFNIKDDYMNWVKQRPEKGLEWIGW
IDPENGDTEYASKFQGRATMTADTSTNTAYMELSSGRSEDTAVYYCTTSN
GWGQGTLVTVSS
SEQ ID NO: 130:
Light chain variable region hu9F5VLv8
DIVMTQSPFSLPVTPGESASISCR5SKSLLHSNGITYLYWYLQKPGQSPQ
LLIYQMSNLASGVPNRFSSSGSGTDFTLKISRVEAEDVGVYYCAQNLELP
LTFGQGTKLEIK
SEQ ID NO:131:
Eight chain variable region hu9F5VLv9 (also
known as hu9F5VLv8_N60D)
DTVMTQSPFSLPVTPGESASISCRSSKSLLKSNGITYLYWYLQKPGQSPQ
LLIYQMSNLASGVPDRFSSSGSGTDFTLKISRVEAEDVGVYYCAQNLELP
LTFGQGTKLEIK
SEQ ID NO: 132: Variant of hu9F5VLv8 light chain
variable region (hu9F5VLv8_V3Q, L27cS, L37Q, M51G,
L54G, L921, also known as hu9F5VLv8_DIM1)
DIQMTQSPFSLPVIPGESASISCRSSKSLSHSNGITYLYWYQQKPGQSPQ
LLIYQGSNGASTIPNRFSSSGSGTDFILKISPATEAEDVGVYYCAQNIEL
PLIFGQGTKLEIK
SEQ ID NO: 133: Variant of hu9F5VLv8 light chain
variable region (hu9F5VLv8_V3Q, L27cS, L37Q, M51G,
L54R, L921, also known as hu9FEATLv_8DIM2)
DIQMIQSPFSLPVTPGESASISCRSSKSLSHSNGITYLYWIQQKPGQSPQ
LLIWGSNRASGVPNRIFSSSGSGTDFILKISRVEAEDVGVYYCAQNIELP
LTFGQTLEIK
SEQ ID NO: 134:
Variant of hu9F5VLv8 light chain variable region
(hu9F5VLv8_V3Q, L27cS, L37Q, M51G, L54T, L921,
also known as hu9F5VLv8_DIM3)
DIQMTQSPFSLPVTPGESASISCRSSKSLSHSNGITYLYWYQQKPGQSP
QLLIYQGSNTASGVPNRFSSSGSGTDFTLKISRVEAEDVGVYYCAQNIE
LPLTFGQGTKLEIK
SEQ ID NO: 135: Variant of hu9F5VLv8 light chain
variable region
(hu9F5VLv8_V3Q, L27cS, L37Q, M51G, L54R, L92G,
also known as hu9F5VLv8_DIM4)
DIQMTQPFSLPVTPGESASISCRSSKSLSHSNGITYLYWYQQKPGQSPQ
LLTYQGSNRASGVPNRESSSGSGTDFTLKISRVEAEDVGVYYCAQNGEL
PLITGQGTKLEIK
SEQ ID NO: 136: Variant of hu9F5VLv8 light chain
variable region
(hu9F5VLv8_V3Q, L27cG, L37Q, M51G, L54R, L921,
also known as hu9F5VLv8_DIM5)
DIQMTQSPFSLPVTPGESASISCRSSKSLGHSNGITYLYWYQQKPGQS
PQLLIYQGSNRASGVPNRFSSSGSGTDFTLKISRVEAEDVGVYYCAQN
IELPLTFGQGTKLEIK
SEQ ID NO: 137: Variant of hu9F5VLv8 light chain
variable region
(hu9F5VLv8_V3Q, L27cD, L37Q, M51G, L54R, L921,
also known as hu9F5VLv8_DIM6)
DIQMTQPFSLPVTPGESASISCRSSKSLDHSNGITYLIWYQQKPGQS
PQLLIWGSNRPLSGVPNRFSSSGSGTDFTLKISRVEAEDVGVYYCAQ
NIELPLTFGGTKLEIK
SEQ ID NO: 138: Variant of hu9F5VLv8 light chain
variable region
(hu9F5VLv8_V3Q, L27cD, L37Q, M51K, L54R, L921,
also known as hu9F5VLv8_DIM7)
DIQMTQSPFSLPVTPGESASISCRSSKSLDHSNGITYLYWYQQKPGQ
SPQLLIYQKSNRASGVPNRFSSSGSGTDFTLKISRVEAEDVGVYYCA
QNIELPLTFGQGTKLEIK
SEQ ID NO: 139: Variant of hu9F5VLv8 light chain
variable region
(hu9F5VLv8_V3Q, L27cG, L37Q, M51K, L54R, L921,
also known as hu9F5VLv8_DIM8)
DIQMTQSPFSLPVTPGESASISCRSSKSLGHSNGITYLYWYQQKPGQ
SPQLLIYQKSNRASGVPNRFSSSGSGTDFTLKISRVEAEDVGVYYCA
QNIELPLTFGQGTKLEIK
SEQ ID NO: 140: Variant of hu9F5VLv8 light chain
variable region
(hu9F5VLv8_V3Q, L27cG, L37Q, M51K, L54G, L921,
also known as hu9F5VLv8_DIM9)
DIQMTQ5PFSLPVTPGESASISCRSSKSLGHSNGTTYLYWYQQKPG
QSPQLLIYQKSNGASGVPNRFSSSGSGTDFTLKISRVEAEDVGVYY
CAQNIELPLTFGQGTKLEIK
SEQ ID NO: 141:
Variant of hu9F5VLv8 light chain variable region
(hu9F5VLv8_V3Q, L27cS, L37Q, M51K, L54G, L92I,
also known as hu9F5VLv8_DIM10)
DIQMTQSPFSLPVTPGESASISCRSSKSLSHSMGITYLYWYQQKPGQSPQ
LLIYQKSMGASGVPNRFSSSGSGTDFTLKISRVEAEDVGVYYCAQNIELP
LTFGQGTKLEIK
SEQ ID NO: 142:
Variant of hu9F5VLv8 light chain variable region
(hu9F5VLv8_V3Q, L27cG, L37G, M51G, L54R, L92I,
also known as hu9F5VLv8_DIH11)
DIQMTQSPFSLFVTPGESASISCRSSKSLGHSNGITYLYWYGQKPGQSPQ
LLIYQGSNRASGVPNRFSSSGSGTDFTLKISRVEAEDVGVYYCAQNIELP
LTFGQGTKLEIK
SEQ ID NO: 143:
Variant ofhu9F5VLv8 light chain variable region
(hu9F5VLv8_V3Q, L27cG, L37G, M51G, L54R, L92G,
also known as hu9F5VLv8_DIM12)
DIQMTQSPFSLPVTPGSSASISCRSSKSLGHSNGITYLYWYGQKPGQSPQ
LLIYQGSMRASGVPNRFSSSGSGTDFTLKISRVEAEDVGVYYCAQNGELP
LTFGQGTKLEIK
SEQ ID NO: 144:
Variant of hu9F5VLv8 light chain variable region
(hu9F5VLv8_V3Q, L27cG, L37G, M51G, L54R, also
known as hu9F5VLv8_DIM13)
EIQMTQSPFSLPVTPGESASISCRSSKSLGKSKGITYLYWYGQKPGQSPQ
LLIYQGSNRASGVPNRFSSSGSGTDFTLKISRVEAEDVGVYYGAQNLELP
LTFGQGTKLEIK
SEQ ID NO: 145:
Variant ofhu9F5VLv8 light chain variable region
(hu9F5VLv8_V3Q, L27cG, L37G, M51G, L54T, L92I,
also known as hu9F5VLv8_DIM14)
DIQMTQSPFSLPVTPGESASISCRSSKSLGKSKGITYLYWYGQKPGQSPQ
LLIYQGSNTASGVPNRFSSSGSGTDFTLKISRVEAEDVGVYYCAQNIELP
LTFGQGTKLEIK
SEQ ID NO: 146:
Variant of hu9F5VLv8 light chain variable region
(hu9F5VLv8_V3Q, L27cG, L37G, M51G, L54T, L92G,
also known as hu9F5VLv8_DIM15)
DIQMTQSPFSLPVTPGESASISCRSSKSLGHSKGITYLYWYGQKPGQSPQ
LLIYQGSMTASGVPNRFSSSGSGTDFTLKISRVEAEDVGVYYCAQNGELP
LTFGQGTKLEIK
SEQ ID NO: 147:
Variant of hu9F5VLv8 light chain variable region
(hu9F5VLv8_V3Q, L27cG, L37G, M51G, L54T, also
known as hu9F5VLv8_DIM16)
DIQMTQSPFSLPVTPGESASISCRSSKSLGKSKGITYLYWYGQKPGQSPQ
LLIYQGSMTASGVPNRFSSSGSGTDFTLKISRVEAEDVGVYYCAQNLELP
LTFGQGTKLEIK
SEQ ID NO: 148:
Variant of hu9F5VLv8 light chain variable region
(hu9F5VLv8_V3Q, L27cS, L37G, M51G, L54T, L92I,
also known as hu9F5VLv8_DIM17)
DIQMTQSPFSLPVTPGESASISCRSSKSLSHSNGITYLYWYGQKPGQSPQ
LLIYQGSNTASGVPNRFSSSGSGTDFTLKISRVEAEDVGVYYCAQNIELP
LTFGQGTKLEIK
SEQ ID NO: 149:
Variant of hu9F5VLv8 light chain variable region
(hu9F5VLv8_V3Q, L27cD, L37G, M51G, L54R, L92I,
also known as hu9F5VLv8_DIM18)
DIQMTQSPFSLPVTPGESASISCRSSKSLDHSNGITYLYWYGQKPGQSPQ
LLIYQGSMPASGVPNRFSSSGSGTDFTLKISRVEAEDVGVYYCAQNIELP
LTFGQGTKLEIK
SEQ ID NO: 150:
Variant of hu9F5VLv8 light chain variable region
(hu9F5VLv8_V3Q, L27cS, L37I, M51I, L54R, L92I,
also known as hu9F5VLv8_DIM19)
DIQMTQSPFSLPVTPGESASISCRSSKSLSHSNGITYLYWYIQKPGQSPQ
LLIYQISMRASGVPNRFSSSGSGTDFTLKISRVEAEDVGVYYCAQNIELP
LTFGQGTKLEIK
SEQ ID NO: 151:
Variant of hu9F5VLv8 light chain variable region
(hu9F5VLv8_V3Q, L27cS, L37Q, M51I, L54G, L92I,
also known as hu9F5VLv8_DIM20)
DIQMTQSPFSLPVTPGESASISCRSSKSLSHSNGITYLYWYQQKPGQSPQ
LLIYQISNGASGVPNRFSSSGSGTDFTLKISRVEAEDVGVYYCAQNIELP
LTFGQGTKLEIK
SEQ ID NO:152:
Variant of hu9F5VLv8 light chain variable region
(hu9F5VLv8_V3Q, L27cS, L37Q, M51I, L54G, also
known as hu9F5VLv8_DIM21)
DIQMTQSPFSLPVTPGESASISCRSSKSLSKSNGITYLYWYQQKPGQSPQ
LLIYQISMGASGVPNRFSSSGSGTDFTLKISRVEAEDVGVYYCAQNLELP
LTFGQGTKLEIK
SEQ ID NO: 153:
Variant of hu9F5VLv8 light chain variable region
(hu9F5VLv8_V3Q, L27cS, L37Q, M51E, L54R, L92I,
also known as hu9F5VLv8_EIM22)
DIQMTQSPFSLPVTPGESASISCRSSKSLSHSNGITYLYWYQQKPGQSPQ
LLIYQESNRASGVPNRFSSSGSGTDFTLKISRVEAEDVGVYYCAQNIELP
LTFGQGTKLEIK
SEQ ID NO: 154
Variant of hu9F5VLv8 light chain variable region
(hu9F5VLv8_V3Q, L27cG, L37Q, M51E, L54G, L92I,
also known as hu9F5VLv8_DTM23)
DIQMTQSPFSLPVTPGESASISCRSSKSLGHSNGITYLYWYQQKPGQSPQ
LLIYQESNGASGVPNRFSSSGSGTDFTLKISRVEAEDVGVYYCAQNIELP
LTFGQGTKLEIK
SEQ ID NO: 155:
Variant of hu9F5VLv8 light chain variable region
(hu9F5VLv8_V3Q, L27cG, L37I, M51E, L54R, L92I,
also known as hu9F5VLv8_DIM24)
DIQMTQSPFSLPVTPGESASISCRSSKSLGHSNGITYLYWYIQKPGQSPQ
LLIYQESNRASGVPNRFSSSGSGTDFTLKISRVEAEDVGVYYCAQNIELP
LTFGQGTKLEIK
SEQ ID NO: 156:
Variant of hu9F5VLv8 light chain variable
region (hu9F5VLv8_V3Q, L27cG, L37I, M51E, L54R,
L92G also known as hu9F5VLv8_DIM25)
DIQMTQSPFSLFVTPGESASISCRSSKSLGHSNGITYLYWYIQKPGQSPQ
LLIYQESNRASGVPGSGTDFTLKISRVEAEDVGVYYCAQNGELPLTFGQG
TKLEIK
SEQ ID NO: 157:
Variant of hu9F5VLv8 light chain variable region
(hu9F5VLv8_V3Q, L27cl, L371, M51E, L54R, also
known as hu9F5VLv8_DIM26)
DIQMTQSPFSLPVTPGESASISCRSSKSLIHSNGITYLYWYIQKPGQSPQ
LLIYQESNRASGVPNRFSSSGSGTDFTLKISRVEAEDVGVYYCAQNLELP
LTFGQGTKLEIK
SEQ ID NO: 158: Variant of hu9F5VLv8 light chain
variable region (hu9F5VLv8_V3Q, L37Q, M51G, L54R,
L92I, also known as hu9F5VLv8 _DIM27)
DTQMTQSPFSLPVTPGESASISCRSSKSLLHSNGITYLYWYQQKPGQSPQ
LLTYQGSNRASGVPNRFSSSGSGTDFTLKISRVEAEDVGVYYCAQNIELP
LTFGQGTKLEIK
SEQ ID NO: 159:
Variant of hu9F5VLv8 light chain variable region
(hu9F5VLv8_V3Q, L27cS, M51G, L54R, L92I, also
known as hu9F5VLv8_DIM28)
DIQMTQSPFSLPVTPGESASISCRSSKSLSHSNGITYLYWYLQKPGQSPQ
LLIYQGSNRASGVPNRFSSSGSGTDFTLKISRVEAEDVGVYYCAQNIELP
LTFGQGTKLEIK
SEQ ID NO:160:
Variant of hu9F5VLv8 light chain variable region
(hu9F5VLv8_V3Q, L27cS, L37Q, L54R, L92I, also
known as hu9F5VLv8_DIM29)
DIQMTQSPFSLPVTPGESASISCRSSKSLSKSNGITYLYWYQQKPGQSPQ
LLIYQMSNRASGVPNRFSSSGSGTDFTLKISRVEAEDVGVYYCAQNIELP
LTFGQGTKLEIK
SEQ ID NO: 161:
Variant of hu9F5VLv8 light chain variable region
(hu9F5VLv8_V3Q, L27cS, L37Q, M51G, L92I, also
known as hu9F5VLv8_DIM30)
DIQMTQSPFSLPVTPGESASISCRSSKSLSKSNGITYLYWYQQKPGQSPQ
LLIYQGSNLASGVPNRFSSSGSGTDFTLKISRVEAEDVGVYYCAQNIELP
LTFGQGTKLEIK
SEQ ID NO: 162:
Variant of hu9F5VLv9 light chain variable region
(hu9F5VLv9_V3Q, L27cS, L37Q, M51G, L54G, L92I,
also known as hu9F5VLv9_DIMl)
DIQMTQSPFSLPVTPGESASISCRSSKSLSHSNGITYLYWYQQKPGQSPQ
LLIYQGSNGASGVPDRFSSSGSGTDFTLKISRVEAEDVGVYYCAQNIELP
LTFGQGTKLEIK
SEQ ID NO: 163:
Variant of hu9F5VLv9 light chain variable region
(hu9F5VLv9_V3Q, L27cS, L37Q, M51G, L54R, L92I,
also known as hu9F5VLv9_DIM2)
DIQMTQSPFSLPVTPGESASISCRSSKSLSHSNGITYLYWYQQKPGQSPQ
LLIYQGSNRASGVPDRFSSSGSGTDFTLKISRVEAEDVGVYYCAQNIELP
LTFGQGTKLEIK
SEQ ID NO: 164:
Variant of hu9F5VLv9 light chain variable region
(hu9F5VLv9_V3Q, L27cS, L37Q, M51G, L54R, L92G,
also known as hu9F5VLv9_DIM4 )
DIQMTQSPFSLPVTPGESASISCRSSKSLSKSNGITYLYWYQQKPGQSPQ
LLIYQGSNRASGVPDRFSSSGSGTDFTLKISRVEAEDVGVYYCAQNGELP
LTFGQGTKLEIK
SEQ ID NO: 165:
Variant of hu9F5VLv9 light chain variable region
(hu9F5VLv9_V3Q, L27cG, L37Q, M51G, L54R, L92I,
also known as hu9F5VLv9 DIMS)
DIQMTQSPFSLPVTPGESASISCRSSKSLGHSNGITYLYWYQQKPGQSPQ
LLIYQGSNRASGVPDRFSSSGSGTDFTLKISRVEAEDVGVYYCAQNIELP
LTFGQGTKLEIK
SEQ ID NO: 166:
Variant ofhu9F5VLv9 light chain variable region
(hu9F5VLv9_V3Q, L27cG, L37Q, M51K, L54R, L92I,
also known as hu9F5VLv9_DIM8)
DIQMTQSPFSLPVTPGESASISCRSSKSLGKSNGITYLYWYQQKPGQSPQ
LLIYQKSNRASGVPDRFSSSGSGTDFTLKISRVEAEDVGVYYCAQNIELP
LTFGQGTKLEIK
SEQ ID NO: 167:
Variant ofhu9F5VLv9 light chain variable region
(hu9F5VLv9_V3Q, L27cS, L37Q, M51K, L54G, L92I,
also known as hu9F5VLv9_DIM10)
DIQMTQSPFSLPVTPGESASISCRSSKSLSKSNGITYLYWYQQKPGQSPQ
LLIYQKSNGASGVIDRFSSSGSGTDFTLKISRVEAEDVGVYYCAQNIELP
LTFGQGTKLEIK
SEQ ID NO: 168:
Variant of hu9F5VLv9 light chain variable region
(hu9F5VLv9_V3Q, L27cG, L37G, M51G, L54R, L92I,
also known as hu9F5VLv9_DIM11)
DIQMTQSPFSLPVTPGESASISCRSSKSLGHSNGITYLYWYGQKPGQSPQ
LLIYQGSNRASGVPDRFSSSGSGTDFTLKISRVEAEDVGVYYCAQNIELP
LTFGQGTKLEIK
SEQ ID NO: 169:
Variant of hu9F5VLv9 light chain variable region
(hu9F5VLv9_V3Q, L27cG, L37G, M51G, L54R, also
known as hu9F5VLv9_DIM13}
DIQMTQSPFSLPVTPGESASISCRSSKSLGHSNGITYLYWYGQKPGQSPQ
LLIYQGSNRASGVPDRFSSSGSGTDFTLKISRVEAEDVGVYYCAQNLELP
LTFGQGTKLEIK
SEQ ID NO: 170:
Variant of hu9F5VLv9 light chain variable region
(hu9F5VLv9_V3Q, L27cS, L37I, M51I, L54R, L92I,
also known as hu9F5VLv9_DIM19)
DIQMTQSPFSLPVTPGESASISCRSSKSLSKSNGITYLYWYIQKPGQSPQ
LLIYQISMRASGVPDRFSSSGSGTDFTLKISRVEAEDVGVYYCAQNIELP
LTFGQGTKLEIK
SEQ ID NO:171:
Variant ofhu9F5VLv9 light chain variable region
(hu9F5VLv9_V3Q, L27cS, L37Q, M51I, L54G, L92I,
also known as hu9F5VLv9_DIM20)
DIQMTQSPFSLPVTPGESASISCRSSKSLSHSNGITYLYWYQQKPGQSPQ
LLIYQISNGASGVPDRFSSSGSGTDFTLKISRVEAEDVGVYYCAQNIELP
LTFGQGTKLEIK
SEQ ID NO: 172
Alternate Kabat CDR-L1 of a humanized 9F5
antibody (L27bD)(present in hu9F5VLv2_L27bD,
SEQ ID NO: 71)
RSSKSDLHSNGITYLY
SEQ ID NO: 173
Alternate Kabat CDR-L1 of a humanized 9F5
antibody (L27bT)
(present in hu9F5VLv2_L27bT, SEQ ID NO: 79)
RSSKSTLHSNGITYLY
SEQ ID NO: 174
Alternate Kabat CDR-L1 of a humanized 9F5
antibody (L27bQ)
(present in hu9F5VLv2_L27bQ, SEQ ID NO:81)
RSSKSQLHSNGITYLY
SEQ ID NO: 175
Alternate Kabat CDR-L1 of
a humanized 9F5 antibody (L27cD)
(present in hu9F5VLv2_L27cD, SEQ ID NO: 63,
in hu9F5VLv8_DIM6, SEQ ID NO: 137, in
hu9F5VLv8_DLM7, SEQ ID NO: 138, and in
hu9F5VLv8_DLM18, SEQ ID NO: 149)
RSSKSLDHSNGITYLY
SEQ ID NO: 176
Alternate Kabat CDR-L1 of a humanized
9F5 antibody (L27cG)
(present in hu9F5VLv2_L27cG, SEQ ID NO: 64,
in hu9F5VLv8_DIM5, SEQ ID NO: 136, in
hu9F5VLv8_DIM8, SEQ ID NO: 139,
in hu9F5VLv8_DIM9, SEQ ID NO: 140, in
hu9F5VLv8_DIM11, SEQ ID NO: 142, in
hu9F5VLv8_DIM12, SEQ ID NO: 143, in
hu9F5VLv8_DIM13, SEQ ID NO: 144, in
hu9F5VLv8_DIMM, SEQ IDNO:145, in
hu9F5VLv8_DIM15, SEQ ID NO: 146, in
hu9F5VLv8_DIM16, SEQ ID NO: 147, in
hu9F5VLv8_DIM23, SEQ ID NO: 154, in
hu9F5VLv8_DIM24, SEQ ID NO: 155, in
hu9F5VLv8_DIM25, SEQ ID NO: 156, in
hu9F5VLv9_DIM5, SEQ ID NO 165, in
hu9F5VLv9_DIM5, SEQ ID NO: 166, in
hu9F5VLv9_DIM11, SEQ ID NO: 168,
and in
hu9F5 VLv9_DIM13, SEQ ID NO: 169)
RSSKSLGHSNGITYLY
SEQ ID NO: 177
Alternate Kabat CDR-LI of a humanized
9F5 antibody (L27cS)
(present in
hu9F5VLv2_L27cS, SEQ ID NO:65, in
hu9F5VLvS_DIM 1, SEQ ID NO: 132, in
hu9F5VLv8_DIM2, SEQ ID NO: 133, in
hu9F5VLv8_DIM3, SEQ ID NO: 134, in
hu9F5VLv8J)IM4, SEQ ID NO. 135, in
hu9F5VLv8JDIM10, SEQ ID NO 141, in
hu9F5VLv8_DIM 17, SEQ ID NO: 148,
hu9F5VLv8_DIM19, SEQ ID NO: 150, in
hu9F5 VLv8_DIM20, SEQ ID NO: 151, in
hu9F5VLv8_DIM23, SEQ ID NO: 152, in
hu9F5VLv8_DIM22, SEQ ID NO: 153, in
hu9F5VLv8_DIM28, SEQ ID NO: 159, in
hu9F5VLv8_DLM29, SEQ ID NO: 160, in
hu9F5VLv8_DIM30, SEQ I NO: 161, in
hu9F5VLv9_DIM 1, SEQ ID NO: 162, in
hu9F5VLv9_DIM2, SEQ ID NO: 163, in
hu9F5VLv9_DIM4, SEQ ID NO. 164, in
hu9F5VLv9_DIM10, SEQ ID NO 167, in
hu9F5VLv9_DIM19, SEQ ID NO: 170, and in
hu9F5VLv9_DIM20, SEQ ID NO: 171)
RSSKSLSHSNGITYLY
SEQ ID NO: 178
Alternate Kabat CDR-L1 of a humanized 9F5
antibody (L27cE)
(present in hu9F5VLv2_L27cE, SEQ ID NO: 66)
RSSKSLEHSNGITYLY
SEQ ID NO: 179 Alternate Kabat CDR-L1 of a
humanized 9F5 antibody (L27cT)
(present in hu9F5VLv2_L27cT, SEQ ID NO: 69)
RSSKSLTHSNGITYLY
SEQ ID NO: 180 Alternate Kabat CDR-L1 of a
humanized 9F5 antibody (L27cN)
(present in hu9F5VLv2_L27cN, SEQ ID NO: 70)
RSSKSLNHSNGITYLY
SEQ ID NO: 181 Alternate Kabat CDR-L1 of a
humanized 9F5 antibody (L27cA)
(present in hu9F5VLv2_L27cA, SEQ ID NO: 74)
RSSKSLAHSNGITYLY
SEQ ID NO: 182 Alternate Kabat CDR-L1 of a
humanized 9F5 antibody (L27cP)
(present in hu9F5VLv2_L27cP, SEQ ID NO: 83)
RSSKSLPHSNGITYLY
SEQ ID NO: 183 Alternate Kabat CDR-L1 of a
humanized 9F5 antibody (L27cI)
(present in hu9F5VLv8_DIM26, SEQ ID NO: 157)
RSSKSLIHSNGITYLY
SEQ ID NO: 184 Alternate Kabat CDR-L1 of a
humanized 9F5 antibody (I30E)
(present in hu9F5VLv2_I30E, SEQ ID NO: 67)
RSSKSLLHSNGETYLY
SEQ ID NO: 185 Alternate Kabat CDR-L1 of a
humanized 9F5 antibody (I30K)
(present in hu9F5VLv2_I30K, SEQ ID NO: 68)
RSSKSLLHSNGKTYLY
SEQ ID NO: 186 Alternate Kabat CDR-L1 of a
humanized 9F5 antibody (I30G)
(present in hu9F5VLv2_I30G, SEQ ID NO: 72)
RSSKSLLHSNGGTYLY
SEQ ID NO: 187 Alternate Kabat CDR-L1 of a
humanized 9F5 antibody (I30Q)
(present in hu9F5VLv2_I30Q, SEQ ID NO: 78)
RSSKSLLHSNGQTYLY
SEQ ID NO: 188 Alternate Kabat CDR-L1 of a
humanized 9F5 antibody (T31G)
(present in hu9F5VLv2_T31G, SEQ ID NO: 80)
RSSKSLLHSNGIGYLY
SEQ ID NO: 189 Alternate Kabat CDR-L1 of a
humanized 9F5 antibody (L33N)
(present in hu9F5VLv2_L33N, SEQ ID NO: 73)
RSSKSLLHSNGITYNY
SEQ ID NO: 190 Alternate Kabat CDR-L1 of a
humanized 9F5 antibody (L33T)
(present in hu9F5VLv2_L33T, SEQ ID NO: 75)
RSSKSLLHSNGITYTY
SEQ ID NO: 191 Alternate Kabat CDR-L1 of a
humanized 9F5 antibody (L335)
(present in hu9F5VLv2_L335, SEQ ID NO: 76)
RSSKSLLHSNGITYSY
SEQ ID NO: 192 Alternate Kabat CDR-L1 of a
humanized 9F5 antibody (L33R)
(present in hu9F5VLv2_L33R, SEQ ID NO: 77)
RSSKSLLHSNGITYRY
SEQ ID NO: 193 Alternate Kabat CDR-L1 of a
humanized 9F5 antibody (L33G)
(present in hu9F5VLv2_L33G, SEQ ID NO: 82)
RSSKSLLHSNGITYGY
SEQ ID NO: 194 Alternate Kabat CDR-L2 of a
humanized 9F5 antibody (M51E)
(present in hu9F5VLv2_M51E, SEQ ID NO: 61)
QESNLAS
SEQ ID NO: 195 Alternate Kabat CDR-L2 of a
humanized 9F5 antibody (M51D)
(present in hu9F5VLv2_M51D, SEQ ID NO: 62)
QDSNLAS
SEQ ID NO: 196 Alternate Kabat CDR-L2 of a
humanized 9F5 antibody (M51G)
(present in hu9F5VLv8_DIM30, SEQ ID NO: 161)
QGSNLAS
SEQ ID NO: 197 Alternate Kabat CDR-L2 of a
humanized 9F5 antibody (L54R)
(present in hu9F5VLv8_DIM29, SEQ ID NO: 160)
QMSNRAS
SEQ ID NO: 198 Alternate Kabat CDR-L2 of a
humanized 9F5 antibody (M51G L54G) (present
in hu9F5VLv8_DIM1, SEQ ID NO: 132,
and in hu9F5VLv9_DIM1, SEQ ID NO: 162)
QGSNGAS
SEQ ID NO: 199 Alternate Kabat CDR-L2 of a
humanized 9F5 antibody (M51G L54T) (present
in hu9F5VLv8_DIM3, SEQ ID NO: 134, in
hu9F5VLv8_DIM14, SEQ ID NO: 145, in
hu9F5VLv8_DIM15, SEQ ID NO: 146, in
hu9F5VLv8_DIM16, SEQ ID NO: 147,
and in hu9F5VLv8_DIM17, SEQ ID NO: 148)
QGSNTAS
SEQ ID NO: 200 Alternate Kabat CDR-L2 of a
humanized 9F5 antibody (M51K L54R) (present
in hu9F5VLv8_DIM7, SEQ ID NO: 138, in
hu9F5VLv8_DIM8, SEQ ID NO: 139, and in
hu9F5VLv9_DIM8, SEQ ID NO: 166)
QKSNRAS
SEQ ID NO: 201 Alternate Kabat CDR-L2 of a
humanized 9F5 antibody (M51K L54G) (present
in hu9F5VLv8_DIM9, SEQ ID NO: 140,
in hu9F5VLv8_DIM10, SEQ ID NO: 141,
and in hu9F5VLv9_DIM10, SEQ ID NO: 167)
QKSNGAS
SEQ ID NO: 202 Alternate Kabat CDR-L2 of a
humanized 9F5 antibody (M51I L54R) (present
in hu9F5VLv8_DIM19, SEQ ID NO: 150, and
in hu9F5VLv9_DIM19, SEQ ID NO: 170)
QISNRAS
SEQ ID NO: 203 Alternate Kabat CDR-L2 of a
humanized 9F5 antibody (M51I L54G) (present
in hu9F5VLv8_DIM20, SEQ ID NO: 151, in
hu9F5VLv8_DIM21, SEQ ID NO: 152 and in
hu9F5VLv9_DIM20, SEQ ID NO: 171)
QISNGAS
SEQ ID NO: 204 Alternate Kabat CDR-L2 of a
humanized 9F5 antibody (M51E L54R) (present
in hu9F5VLv8_DIM22, SEQ ID NO: 153, in
hu9F5VLv8_DIM24, SEQ ID NO: 155,
in hu9F5VLv8_DIM25, SEQ ID NO: 156,
and in hu9F5VLv8_DIM26, SEQ ID NO: 157)
QESNRAS
SEQ ID NO: 205 Alternate Kabat CDR-L2 of a
humanized 9F5 antibody (M51E L54G) (present
in hu9F5VLv8_DIM23, SEQ ID NO: 154)
QESNGAS
SEQ ID NO: 206 Alternate Kabat CDR-L3 of a
humanized 9F5 antibody (A89G)
(present in hu9F5VLv2_A89G , SEQ ID NO: 108)
GQNLELPLT
SEQ ID NO: 207 Alternate Kabat CDR-L3 of a
humanized 9F5 antibody (L92D)
(present in hu9F5VLv2_L92D, SEQ ID NO: 100)
AQNDELPLT
SEQ ID NO: 208 Alternate Kabat CDR-L3 of a
humanized 9F5 antibody (L92E)
(present in hu9F5VLv2_L92E, SEQ ID NO: 102)
AQNEELPLT
SEQ ID NO: 209 Alternate Kabat CDR-L3 of a
humanized 9F5 antibody (L92G)
(present in hu9F5VLv2_L92G, SEQ ID NO: 103,
in hu9F5VLv8_DIM4, SEQ ID NO: 135, in
hu9F5VLv8_DIM12, SEQ ID NO: 143,
in hu9F5VLv8_DIM15, SEQ ID NO: 146,
in hu9F5VLv8_DIM25, SEQ ID NO: 156,
and in hu9F5VLv8_DIM4, SEQ ID NO: 164)
AQNGELPLT
SEQ ID NO: 210 Alternate Kabat CDR-L3 of a
humanized 9F5 antibody (L92Q)
(present in hu9F5VLv2_L92Q, SEQ ID NO: 104)
AQNQELPLT
SEQ ID NO: 211 Alternate Kabat CDR-L3 of a
humanized 9F5 antibody (L92T)
(present in hu9F5VLv2_L92T, SEQ ID NO: 107)
AQNTELPLT
SEQ ID NO: 212 Alternate Kabat CDR-L3 of a
humanized 9F5 antibody (L92I)
(present in hu9F5VLv8_DIM11,
SEQ ID NO: 132, in hu9F5VLv8_DIM2,
SEQ ID NO: 133, in hu9F5VLv8_DIM3,
SEQ ID NO: 134, in hu9F5VLv8_DIM5,
SEQ ID NO: 136, in hu9F5VLv8_DIM6,
SEQ ID NO: 137, in hu9F5VLv8_DIM7,
SEQ ID NO: 1382 in hu9F5VLv8_DIM8,
SEQ ID NO: 139, in hu9F5VLv8_DIM9,
SEQ ID NO 140, hu9F5VLv8_DIM10,
SEQ ID NO: 141, in hu9F5VLv8_DIM1,
SEQ ID NO: 142, in hu9F5VLv8_DIM14,
SEQ ID NO: 145, in hu9F5VLv8_DIM17,
SEQ ID NO: 148, in hu9F5VLv8_DIM18,
SEQ ID NO: 149, in hu9F5VLv8_DIM19,
SEQ ID NO: 150, in hu9F5VLv8_DIM20,
SEQ ID NO: 151, in hu9F5VLv8_DIM22,
SEQ ID NO: 153, in hu9F5VLv8_DIM23,
SEQ ID NO: 154, in hu9F5VLv8_DIM24,
SEQ ID NO: 155, in hu9F5VLv8_DIM27,
SEQ ID M) 18, in hu9F5VLv8_DIM28,
SEQ ID NO: 159, in hu9F5VLv8_DIM29,
SEQ ID NO: 160, in hu9F5VLv8_DIM30,
SEQ ID NO: 161, in hu9F5VLv9_DIM1,
SEQ ID NO: 162, in hu9F5VLv9_DIM2,
SEQ ID NO: 163, in hu9F5VLv9_DIM5,
SEQ ID NO: 165, in hu9F5VLv9_DIM8,
SEQ ID NO: 166, in hu9F5VLv9_DIM10,
SEQ ID NO: 167, in hu9F5VLv9_DIM11,
SEQ ID NO: 168, in hu9F5VLv9_DIM19,
SEQ ID NO: 170, and
in hu9F5VLv8_DIM20, SEQ ID NO: 171)
AQNIELPLT
SEQ ID NO: 213 Alternate Kabat CDR-L3 of a
humanized 9F5 antibody (L93G)
(present in hu9F5VLv2_L93G, SEQ ID NO: 105)
AQNLGLPLT
SEQ ID NO: 214 Heavy chain
variable region hu10C12VHv1
QVQLVQSGAEVKKPGATVKISCKASGFNIKDDYMNWVQQAPGKGLEWIGW
IDPENGDTEYASKFQGRATMTADTSTDTAYMELSSLRSEDTAVYYCTTSN
GWGQGTLVTVSS
SEQ ID NO: 215 Heavy chain
variable region hu10C12VHv2
EVQLVQSGAEVKKPGATVKISCKASGFNIKDDYMNWVQQAPGKGLEWIGW
IDPENGDTEYASKFQGRATMTADTSTDTAYMELSSLRSEDTAVYYCTTSN
GWGQGTLVTVSS
SEQ ID NO: 216 Light chain
variable region hu10C12VLv1
DIVMTQSPLSLPVTPGEPASISCRSSKSLLHSNGITYLYWYLQKPGQSPQ
LLIYQMSNLASGVPDRFSSSGSGTDFTLKISRVEAEDVGVYYCAQNLELP
LTFGGGTKVEIK
SEQ ID NO: 217 Light chain
variable region hu10C12VLv2
DIVMTQSPLSLPVTPGEPASISCRSSKSLLHSNGITYLYWYLQKPGQSPQ
LLIYQMSNLASGVPDRFSSSGSGTDFTLKISRVEAEDVGVYYCAQNLELP
LTFGGGTKLEIK
SEQ ID NO: 218 Heavy chain
variable region acceptor CAC20421-VH_huFrwk
QVQLVQSGAEVKKPGATVKISCKVSGYTFTDYYMEIWVQQAPGKGLEWMG
LVDPEDGETIYAEKFQGRVTITADTSTDTAYMELSSLRSEDTAVYYCARI
PLFGRDHWGQGTLVTVSS
SEQ ID NO: 219 Heavy chain
variable region of mouse 12C4 antibody
EVQLQQSGAELVRPGASVKLSCTASGFNIKDDYMNWVRQRPERGLEWIGW
IDPENGDTAYASKFQGKATMTADTSSNTAYLQFSSLTSEDSAVYYCTTSN
GWGQGTLVTVSA
SEQ ID NO: 220
Kabat CDR-H2 of mouse 12C4 antibody
WIDPENGDTAYASKFQG
SEQ ID NO: 221 Heavy chain
variable region hu12C4VHv1
QVQLVQSGAEVKKPGATVKISCKVSGFNIKDDYMNWVQQAPGKGLEWMGW
IDPENGDTAYASKFQGRVTITADTSTDTAYMELSSLRSEDTAVYYCARSN
GWGQGTLVTVSS
SEQ ID NO: 222 Heavy chain
variable region hu12C4VHv2
EVQLVQSGAEVKKPGATVKISCKVSGFNIKDDYMNWVQQAPGKGLEWIGW
IDPENGDTAYASKFQGRVTITADTSTDTAYMELSSLRSEDTAVYYCTTSN
GWGQGTLVTVSS
SEQ ID NO: 223 Light chain
variable region hu12C4VLv1
DIVMTQSPLSLPVTPGEPASISCRSSKSLLHSNGITYLYWYLQKPGQSPQ
LLIYQMSNLASGVPDRFSGSGSGTDFTLKISRVEAEDVGVYYCAQNLELP
LTFGGGTKVEIK
SEQ ID NO: 224_Light chain
variable region hu12C4VLv2
DIVMTQSPLSLPVTPGEPASISCRSSKSLLHSNGITYLYWYLQKPGQSPQ
LLIYQMSNLASGVPDRFSSSGSGTDFTLKISRVEAEDVGVYYCAQNLELP
LTFGGGTKLEIK
SEQ ID NO: 225 Heavy chain
variable region of mouse 17C12 antibody
KIQLQQSGAELVRPGASVKLSCTASAFNIKDDYMNWVKQRPERGLEWIGW
IDPENGDTKYASKFQGKATMTADTSSNTAYLQLSSLTSEDTAVYYCTTSN
GWGQGTLVTVSA
SEQ ID NO: 226 Kabat-Chothia composite CDR H1
of mouse 17C12 antibody
AFNIKDDYMN
SEQ ID NO: 227 Kabat CDR H2 of mouse 17C12 antibody
WIDPENGDTKYASKFQG
SEQ ID NO: 228 Light chain
variable region of mouse 17C12 antibody
DVVMTQTPLTLSVTIGQPASISCTSSQSLLHSNRKTYLHWLLQRPGQSPK
WYLVSKLESGVPDRFSGSGSGTDFTLKISRVEAEDLGVYYCLQTTHFPRT
FGGGTKLEIK
SEQ ID NO: 229 Kabat CDR-L1 of mouse 17C12 antibody
TSSQSLLHSNRKTYLH
SEQ ID NO: 230 Kabat CDR-L2 of mouse 17C12 antibody
LVSKLES
SEQ ID NO: 231 Kabat CDR-L3 of mouse 17C12 antibody
LQTTHFPRT
SEQ ID NO: 232 Heavy chain
variable region hu17C12VHv1
QIQLVQSGAEVKKPGATVKISCKASAFNIKDDYMNWVQQAPGKGLEWIGW
IDPENGDTKYASKFQGRATMTADTSTDTAYMELSSLRSEDTAVYYCTTSN
GWGQGTTVTVSR
SEQ ID NO: 233 Heavy chain
variable region hu17C12VHv2
EVQLQQSGAELVKPGATVKISCTASGFNIKDDYMNWVKQRPEQGLEWIGW
IDPENGDTEYASKFQGKATMTADTSTNTAYLQLSSLTSEDTAVYYCTTSN
GWGQGTTVTVSS
SEQ ID NO: 234_Light chain
variable region hu17C12VLv1
DVVMTQTPLSLSVTPGQPASISCTSSQSLLHSNRKTYLHWLLQKPGQPPQ
LLIYLVSKLESGVPDRFSGSGSGTDFTLKISRVEAEDVGVYYCLQTTHFP
RTFGGGTKVEIK
SEQ ID NO: 235 Light chain
variable region hu17C12VLv2
DVVMTQTPLSLSVTPGQPASISCTSSQSLLHSNRKTYLHWLLQKPGQSPQ
LLIYLVSKLE SGVPDRFSGSGSGTDFTLKISRVEAEDVGVYYCLQTTHF
PRTFGGGTKVEIK
SEQ ID NO: 236 Heavy chain
variable region structural model 3PP3-VH mSt
QVQLVQSGAEVKKPGSSVKVSCKASGYAFSYSWINWVRQAPGQGLEWMGR
IFPGDGDTDYNGKFKGRVTITADKSTSTAYMELSSLRSEDTAVYYCARNV
FDGYWLVYWGQGTLVTVSS
SEQ ID NO: 237 Light chain
variable region structural model 3PP3-VL_mSt
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIK
SEQ ID NO: 238 Light chain
variable region acceptor QD016713-VL_huFrwk
DIVMTQTPLSLSVTPGQPASISCKSSQSLLHSDGKTYLYWYLQKPGQPPQ
LLIYEVSNRFSGVPDRFSGSGSGTDFTLKISRVEAEDVGVYYCMQSIQLP
PTFGGGTKVEIK
SEQ ID NO: 239 Light chain
variable region germline sequence IGKV2-29*02 &
IGKJ4 *01
DIVMTQTPLSLSVTPGQPASISCKSSQSLLHSDGKTYLYWYLQKPGQSPQ
LLIYEVSSRFSGVPDRFSGSGSGTDFTLKISRVEAEDVGVYYCMQGIHLP
LTFGGGTKVEIK
SEQ ID NO: 240 Heavy chain
variable region of mouse 14H3 antibody
QVTLKESGPGILQPSQTLSLTCSFSGFSLSTYGMGVGWIRQPSGKGLEWL
ANIWWDDIKYYNAALKSRLTISKDTSKNQVFLKIASVDTADTATYYCARN
VDYWGQGTTLTVSS
SEQ ID NO: 241 Kabat-Chothia composite CDR H1
of mouse 14H3 antibody
GFSLSTYGMGVG
SEQ ID NO: 242 Kabat CDR H2 of mouse 14H3 antibody
NIWWDDIKYYNAALKS
SEQ ID NO: 243 Kabat CDR H3 of mouse 14H3 antibody
NVDY
SEQ ID NO: 244_Light chain
variable region of mouse ml4H3 antibody
DVVMTQTPLSLPVSLGDQASISCRSSQSLVHSNGNTFLHWYLQKPGQSPK
LLIYKVSNRFSGVPDRFSGSGSGTDFTLKISRVEAEDLGVYFCSQSTLVP
WTFGGGTKLEIK
SEQ ID NO: 245
Kabat CDR Ll of mouse 14H3 antibody
RSSQSLVHSNGNTFLH
SEQ ID NO: 246
Kabat CDR L2 of mouse 14H3 antibody
KVSNRFS
SEQ ID NO: 247
Kabat CDR L3 of mouse 14H3 antibody
SQSTLVPWT
SEQ ID NO: 248 Heavy chain
variable region hul4H3VHvl
QVTLKESGPALVKPTQTLTLTCTFSGFSLSTYGMGVSWIRQPPGKALEWL
ANIWWDDIKYYNAALKSRLTISKDTSKNQVVLTMTNMDPVDTATYYCARN
VDYWGQGTMVTVSL
SEQ ID NO: 249 Heavy chain
variable region hul4H3VHv2
QVTLKESGPALVKPTQTLTLTCTFSGFSLSTYGMGVSWIRQPPGKALEWL
ANIWWDDIKYYNAALKSRLTISKDTSKNQVVLTMTNMDPVDTATYYCARN
VDYWGQGTLVTVSS
SEQ ID NO: 250 Light chain
variable region hul4H3VLv1
DVVMTQTPLSLPVTPGEPASISCRSSQSLVHSNGNTFLHWYLQKPGQSPQ
LLIYKVSNRFSGVPDRFSGSGSGTDFTLKISRVEAEDVGVYFCSQSTLVP
WTFGGGTKVEIK
SEQ ID NO: 251 Light chain
variable region hul4H3VLv2
DVVMTQSPLSLPVTPGEPASISCRSSQSLVHSNGNTFLHWYQQKPGQSPQ
LLIYKVSNRFSGVPDRFSGSGSGTDFTLKISRVEAEDVGVYFCSQSTLVP
WTFGQGTKLEIK
SEQ ID NO: 252 Heavy chain
variable region structural model 2VQ1-VH_mSt
QITLEESGPGILQPSQTLSLTCSFSGFSLSSSAMSVGWIRQPSGKGLEWL
AHIWWNDDKYYNPALKSRLTVSKDSSDNQVFLKIASVVTADTATYYCARI
PGFGFDYWGQGTTLTVSS
SEQ ID NO: 253 Heavy chain
variable region acceptor QDJ57937-VH_huFrwk
QVTLKESGPALVKPTQTLTLTCTFSGFSLSTSGMRVSWIRQPPGKALEWL
ARIDWDDDKFYSTSLKTRLTISKDTSKNQVVLTMTNMDPVDTATYYCARL
AVADAFDIWGQGTMVTVSL
SEQ ID NO: 254 Heavy chain
variable region germline sequence IGHV1-70*04 &
IGHJ4*01
QVTLKESGPALVKPTQTLTLTCTFSGFSLSTSGMRVSWIRQPPGKALEWL
ARIDWDDDKFYSTSLKTRLTISKDTSKNQVVLTMTNMDPVDTATYYCARN
DYWGQGTLVTVSS
SEQ ID NO: 255 Light chain
variable region structural model 2VQ1-VL_mSt
DVVMTQTPLSLSVSLGDQASISCRSSQSLVHSNGNTFLQWYLQKPGQSPK
LLIYKVSNRFSGVPDRFSGSGSGTDFTLKISRMEAEDLGIYFCSQTTHVP
WTFGGGTKLEIK
SEQ ID NO: 256 Light chain
variable region acceptor ABC66914-VL_huFrwk
DIVMTQTPLSLPVTPGEPASISCRSSQSLLHSNGYNYLDWYLQKPGQSPQ
LLIYLGSNRASGVPDRFSGSGSGTDFTLKISRVEAEDVGVYYCMQALQTP
LTFGGGTKVEIK
SEQ ID NO: 257
AbM CDR-H2 of mouse 12C4 antibody
WIDPENGDTA
SEQ ID NO: 258
Contact CDR-H2 of mouse 12C4 antibody
WIGWIDPENGDTA
SEQ ID NO: 259
Chothia CDR-H1 of mouse 17C12 antibody
AFNIKDD
SEQ ID NO: 260
AbM CDR-H2 of mouse 17C12 antibody
WIDPENGDTK
SEQ ID NO: 261
Contact CDR-H2 of mouse 17C12 antibody
WIGWIDPENGDTK
SEQ ID NO: 262
Contact CDR-L1 of mouse 17C12 antibody
KTYLHWL
SEQ ID NO: 263
Contact CDR-L2 of mouse 17C12 antibody
LLIYLVSKLE
SEQ ID NO: 264
Contact CDR-L3 of mouse 17C12 antibody
LQTTHFPR
SEQ ID NO: 265
Kabat CDR-H1 of mouse 14H3 antibody
TYGMGVG
SEQ ID NO: 266
Chothia CDR-H1 of mouse 14H3 antibody
GFSLSTYGM
SEQ ID NO: 267
Chothia CDR-H2 of mouse 14H3 antibody
WWDDI
SEQ ID NO: 268
AbM CDR-H2 of mouse 14H3 antibody
NIWWDDIKY
SEQ ID NO: 269
Contact CDR-H1 of mouse 14H3 antibody
STYGMGVG
SEQ ID NO: 270
Contact CDR-H2 of mouse 14H3 antibody
WLANIWWDDIKY
SEQ ID NO: 271
Contact CDR-H3 of mouse 14H3 antibody
ARNVD
SEQ ID NO: 272
Contact CDR-L1 of mouse 14H3 antibody
NTFLHWY
SEQ ID NO: 273
Contact CDR-L2 of mouse 14H3 antibody
LLIYKVSNRF
SEQ ID NO: 274
Contact CDR-L3 of mouse 14H3 antibody
SQSTLVPW
SEQ ID NO: 275
Alternate Kabat-Chothia composite CDR H1
as in hu14H3VHv1 and hu14H3VHv2
GFSLSTYGMGVS
SEQ ID NO: 276 Consensus motif of a peptide
bound by antibody 9F5, 10C12, 2D11, 12C4,
17C12, and 14H3
IVYK
SEQ ID NO: 277
Consensus motif of a peptide bound by
antibody 2D11
EIVYKS

1. An isolated monoclonal antibody that competes for binding to human tau with antibody 9F5.

2-5. (canceled)

6. The antibody of claim 1 that binds to the same epitope on human tau as 9F5.

7. The antibody of claim 1, comprising three light chain CDRs and three heavy chain CDRs of a mouse antibody characterized by a heavy chain variable region having an amino acid sequence comprising SEQ ID NO:7 and a light chain variable region having an amino acid sequence comprising SEQ ID NO:11.

8. The antibody of claim 1, wherein the three heavy chain CDRs CDR-H1, CDR-H2, and CDR-H3are as defined by Kabat/Chothia Composite (SEQ ID NOs:8, 9, and 10, respectively), except that position H28 can be occupied by N or T, position H51 can be occupied by I or V, position H54 can be occupied by N or D, and position H56 can be occupied by D or E, and the three light chain CDRs CDR-L1, CDR-L2, and CDR-L3 are as defined by Kabat/Chothia Composite (SEQ ID NOs:12, 13, and 14, respectively), except that position L27b is occupied by L, D, T, or Q, position L27c is occupied by L, D, G, S, E, T, N, A, P, or I, position L30 can be occupied by I, Y, E, K, G, or Q, position L31 can be occupied by T, N, or G, position L33 is occupied by L N, T, S, R, or G, position L51 can be occupied by M, G, E, D, K, or I, position L54 can be occupied by L, R, G, or T, position L89 is occupied by A or G, position L92 is occupied by L, D, E, G, Q, T, or I, and position L93 is occupied by E or G.

9. The antibody of claim 8, wherein CDR-H1 has an amino acid sequence comprising SEQ ID NO:50, CDR-H2 has an amino acid sequence comprising SEQ ID NO:51 or SEQ ID NO:52, CDR-L1 has an amino acid sequence comprising any of SEQ ID NO:53, SEQ ID NO:54, and SEQ ID NOs:172-193, wherein CDR-L2 has an amino acid sequence comprising any of SEQ ID NO:55 and SEQ ID NOs:194-205, and wherein CDR-L3 has an amino acid sequence comprising any of SEQ ID NOs:206-213.

10-17. (canceled)

18. The antibody of claim 8, wherein the antibody is a humanized antibody.

19. The humanized antibody of claim 18 wherein the humanized mature heavy chain variable region comprises the three Kabat heavy chain CDRs SEQ ID NO:40, SEQ ID NO:9, and SEQ ID NO:10 and the humanized mature light chain variable region comprises the three Kabat light chain CDRs of SEQ ID NOs:12-14.

20. The humanized antibody of claim 18 comprising a humanized mature heavy chain variable region having an amino acid sequence at least 90% identical to any one of SEQ ID NOs:15-22 and SEQ ID NOs:109-129, and a humanized mature light chain variable region having an amino acid sequence at least 90% identical to any one of SEQ ID NOs:23-29, SEQ ID NOs:61-108, and SEQ ID NOs:130-171.

21. The humanized antibody of claim 20 wherein at least one of the following positions in the VH region is occupied by the amino acid as specified: H1 is occupied by Q or E, H5 is occupied by Q or V, H11 is occupied by L or V, H12 is occupied by V or K, H17 is occupied by S or T, H20 is occupied by L or I, H23 is occupied by T or K, H28 is occupied by N or T, H38 is occupied by K, R, or Q, H40 is occupied by R or A, H42 is occupied by E or G, H43 is occupied by Q or K, H48 is occupied by I or M, H51 is occupied by I or V, H54 is occupied by N or D, H56 is occupied by D or E, H66 is occupied by K or R, H69 is occupied by I or M, H75 is occupied by S or T, H76 is occupied by N or D, H79 is occupied by Y, Q, D, N, or G, H80 is occupied by L, M, P, D, G, or E, H81 is occupied by Q or E, H82 is occupied by L, P, K, R, E, or N, H82a is occupied by S or G, H82c is occupied by L, G, D, or S, H83 is occupied by T or R, H93 is occupied by A or T, H94 is occupied by S or T, H108 is occupied by T or L, H109 is occupied by L or V.

22-25. (canceled)

26. The humanized antibody of claim 20, wherein at least one of the following positions in the VL region is occupied by the amino acid as specified: L3 is V or Q, L7 is A or S, L8 is A or P, L9 is F or L, L11 is N or L, L15 is L or P, L17 is T or E, L18 is S or P, L27b is L, D. T, or Q, L27c is L, D, G, S, E, T, N, A, P, or I, L30 is I,Y, E, K, G, L31 is T, N, or G, L33 is L, N, T, S, R, or G, L37 is L, Q, G, or I, L39 is R or K, L51 is M, G, E, D, K, or I, L54 is R, G, or T, L60 is N or D, L64 is G or S, L66 is E or G, L73 is L, P, or G, L74 is R or K, L75 is I, D, P, Q, or G, L76 is S, P, or G, L77 is R or D, L78 is V, R, D, E, P, K, G, or Q, L85 is V or G, L86 is Y or T, L89 is A or G, L92 is L, D, E, G, Q, T, or I, L93 is E or G, L100 is G or Q.

27-42. (canceled)

43. The humanized antibody of claim 20 wherein the mature heavy chain variable region has an amino acid sequence of any one of SEQ ID NOs:15-22 and SEQ ID NOs:109-129, and the mature light chain variable region has an amino acid sequence of any one of SEQ ID NO:23-29, SEQ ID NOs:61-108, and SEQ ID NOs:130-171.

44-59. (canceled)

60. The antibody of claim 1, comprising three light chain CDRs and three heavy chain CDRs of a mouse antibody characterized by a heavy chain variable region having an amino acid sequence comprising SEQ ID NO:7 and a light chain variable region having an amino acid sequence comprising SEQ ID NO:11.

61. The antibody of claim 60, wherein the antibody is a humanized antibody.

62. The humanized antibody of claim 61 wherein the humanized mature heavy chain variable region comprises the three Kabat heavy chain CDRs SEQ ID NO:40, SEQ ID NO:9, and SEQ ID NO:10 and the humanized mature light chain variable region comprises the three Kabat light chain CDRs SEQ ID NOs:12-14.

63. The humanized antibody of claim 61 comprising a humanized mature heavy chain variable region having an amino acid sequence at least 90% identical to any one of SEQ ID NOs:214-215 and a humanized mature light chain variable region having an amino acid sequence at least 90% identical to any one of SEQ ID NOs:216-217.

64. The humanized antibody of claim 63 wherein at least one of the following positions in the VH region is occupied by the amino acid as specified: H1 is occupied by Q or E, H24 is occupied by A, H48 is occupied by I, H67 is occupied by A, H69 is occupied by M, H93 is occupied by T, H94 is occupied by T.

65. (canceled)

66. The humanized antibody of claim 63, wherein at least one of the following positions in the VL region is occupied by the amino acid as specified: L64 is S, L104 is V or L.

67. (canceled)

68. The humanized antibody of claim 63 wherein the mature heavy chain variable region has an amino acid sequence of any one of SEQ ID NOs:214-215 and the mature light chain variable region has an amino acid sequence of any one of SEQ ID NO:216-217.

69. The antibody of claim 1, comprising three light chain CDRs and three heavy chain CDRs of a mouse antibody characterized by a heavy chain variable region having an amino acid sequence comprising SEQ ID NO:219 and a light chain variable region having an amino acid sequence comprising SEQ ID NO:11.

70. The antibody of claim 69, wherein the antibody is a humanized antibody.

71. The humanized antibody of claim 70 wherein the humanized mature heavy chain variable region comprises the three Kabat heavy chain CDRs SEQ ID NO:40, SEQ ID NO:220, and SEQ ID NO:10 and the humanized mature light chain variable region comprises the three Kabat light chain CDRs SEQ ID NOs:12-14.

72. The humanized antibody of claim 70 comprising a humanized mature heavy chain variable region having an amino acid sequence at least 90% identical to any one of SEQ ID NOs:221-222 and a humanized mature light chain variable region having an amino acid sequence at least 90% identical to any one of SEQ ID NOs:223-224.

73. The humanized antibody of claim 72 wherein at least one of the following positions in the VH region is occupied by the amino acid as specified: H1 is occupied by Q or E, H48 is occupied by M or I, H93 is occupied by A or T, H94 is occupied by R or T.

74. (canceled)

75. The humanized antibody of claim 72, wherein positions in the VL region is occupied by the amino acid as specified: L64 is G or S, L104 is V or L.

76. (canceled)

77. The humanized antibody of claim 72 wherein the mature heavy chain variable region has an amino acid sequence of any one of SEQ ID NOs:221-222 and the mature light chain variable region has an amino acid sequence of any one of SEQ ID NO:223-224.

78. The antibody of claim 1, comprising three light chain CDRs and three heavy chain CDRs of a mouse antibody characterized by a heavy chain variable region having an amino acid sequence comprising SEQ ID NO:225 and a light chain variable region having an amino acid sequence comprising SEQ ID NO:228.

79. The antibody of claim 78, wherein the antibody is a humanized antibody.

80. The humanized antibody of claim 79 wherein the humanized mature heavy chain variable region comprises the three Kabat heavy chain CDRs SEQ ID NO:40, SEQ ID NO:227, and SEQ ID NO:10 and the humanized mature light chain variable region comprises the three Kabat light chain CDRs SEQ ID NOs:229-231.

81. The humanized antibody of claim 79 comprising a humanized mature heavy chain variable region having an amino acid sequence at least 90% identical to any one of SEQ ID NOs:232-233 and a humanized mature light chain variable region having an amino acid sequence at least 90% identical to any one of SEQ ID NOs:234-235.

82. The humanized antibody of claim 81 wherein at least one of the following positions in the VH region is occupied by the amino acid as specified: H1 is occupied by Q or E, H2 is occupied by I, H24 is occupied by A, H48 is occupied by I, H67 is occupied by A, H69 is occupied by M, H93 is occupied by T, H94 is occupied by T, H108 is occupied by T or L, H113 is occupied by R or S.

83. (canceled)

84. The humanized antibody of claim 81, wherein at least one of the following positions in the VL region is occupied by the amino acid as specified: L2 is V, L36 is L, L43 is P or S.

85. (canceled)

86. The humanized antibody of claim 81 wherein the mature heavy chain variable region has an amino acid sequence of any one of SEQ ID NOs:232-233and the mature light chain variable region has an amino acid sequence of any one of SEQ ID NO:234-235.

87. The antibody of claim 1, comprising three light chain CDRs and three heavy chain CDRs of a mouse antibody characterized by a heavy chain variable region having an amino acid sequence comprising SEQ ID NO:240 and a light chain variable region having an amino acid sequence comprising SEQ ID NO:244.

88. The antibody of claim 87, wherein the three heavy chain CDRs CDR-H1, CDR-H2, and CDR-H3 are as defined by Kabat/Chothia Composite (SEQ ID NOs:241, 242, and 243, respectively), except that position H35B can be occupied by G or S, and the three light chain CDRs CDR-L1, CDR-L2, and CDR-L3 are as defined by Kabat/Chothia Composite (SEQ ID NOs:245, 246, and 247, respectively).

89. The antibody of claim 88, wherein CDR-H1 has an amino acid sequence comprising SEQ ID NO:277.

90. The antibody of claim 87, wherein the antibody is a humanized antibody.

91. The humanized antibody of claim 90 wherein the humanized mature heavy chain variable region comprises the three Kabat heavy chain CDRs SEQ ID NO:265, SEQ ID NO:242, and SEQ ID NO:243 and the humanized mature light chain variable region comprises the three Kabat light chain CDRs of SEQ ID NOs:245-247.

92. The humanized antibody of claim 90 comprising a humanized mature heavy chain variable region having an amino acid sequence at least 90% identical to any one of SEQ ID NOs:248-249 and a humanized mature light chain variable region having an amino acid sequence at least 90% identical to any one of SEQ ID NOs:250-251.

93. The humanized antibody of claim 92 wherein at least one of the following positions in the VH region is occupied by the amino acid as specified: H35B is occupied by S, H108 is occupied by M or L, H113 is occupied by L or S.

94. (canceled)

95. The humanized antibody of claim 92, wherein at least one of the following positions in the VL region is occupied by the amino acid as specified: L2 is V, L7 is T or S, L37 is L or Q, L87 is F, L100 is G or Q, L104 is V or L.

96. (canceled)

97. The humanized antibody of claim 92, wherein the mature heavy chain variable region has an amino acid sequence of any one of SEQ ID NOs:248-249 and the mature light chain variable region has an amino acid sequence of any one of SEQ ID NO:250-251.

98. The antibody of claim 1 that is an intact antibody.

99. The antibody of claim 1, that is a binding fragment.

100. The antibody of claim 1, wherein the isotype is human IgG1.

101. The antibody of claim 1 wherein the mature light chain variable region is fused to a light chain constant region and the mature heavy chain variable region is fused to a heavy chain constant region.

102. The antibody of claim 101, wherein the heavy chain constant region is a mutant form of a natural human heavy chain constant region which has reduced binding to a Fcγ receptor relative to the natural human heavy chain constant region.

103. The antibody of claim 1 wherein the isotype is of human IgG2 or IgG4 isotype.

104. A pharmaceutical composition comprising an antibody as defined in claim 1 and a pharmaceutically-acceptable carrier.

105. A nucleic acid encoding the heavy chain and/or light chain of an antibody as described in claim 1.

106. A method of humanizing a mouse antibody, the method comprising:
(a) selecting one or more acceptor antibody sequences;
(b) identifying amino acid residues of the mouse antibody to be retained;
(c) synthesizing a nucleic acid encoding a humanized heavy chain comprising CDRs of the mouse antibody heavy chain and a nucleic acid encoding a humanized light chain comprising CDRs of the mouse antibody light chain; and
(d) expressing the nucleic acids in a host cell to produce a humanized antibody;
wherein the mouse antibody is characterized by a mature heavy chain variable region of SEQ ID NO:7 and a mature light chain variable region of SEQ ID NO:11,
wherein the mouse antibody is characterized by a mature heavy chain variable region of SEQ ID NO:7 and a mature light chain variable region of SEQ ID NO:11,
wherein the mouse antibody is characterized by a mature heavy chain variable region of SEQ ID NO:7 and a mature light chain variable region of SEQ ID NO:11,
wherein the mouse antibody is characterized by a mature heavy chain variable region of SEQ ID NO:219 and a mature light chain variable region of SEQ ID NO:11,
wherein the mouse antibody is characterized by a mature heavy chain variable region of SEQ ID NO:225 and a mature light chain variable region of SEQ ID NO:228,
wherein the mouse antibody is characterized by a mature heavy chain variable region of SEQ ID NO:240 and a mature light chain variable region of SEQ ID NO:244.

107. A method of inhibiting or reducing aggregation of tau in a subject having or at risk of developing a tau-mediated amyloidosis, comprising administering to the subject an effective regime of the antibody of claim 1, thereby inhibiting or reducing aggregation of tau in the subject.

108. A method of treating or effecting prophylaxis of a tau-related disease in a subject, comprising administering an effective regime of an antibody as defined by claim 1 and thereby treating or effecting prophylaxis of the disease.

109. A method of claim 108 wherein the tau-related disease is Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), Parkinson's disease, or progressive supranuclear palsy (PSP).

110. (canceled)

111. A method of detecting tau protein deposits in a subject having or at risk of a disease associated with tau aggregation or deposition, comprising administering to a subject an antibody defined by claim 1, and detecting the antibody bound to tau in the subject.

112. An isolated monoclonal antibody that specifically binds to a peptide consisting of residues (Q/E)IVYK(S/P) (SEQ ID NO:56)), QIVYKP (SEQ ID NO:57), EIVYKSP (SEQ ID NO:58), or EIVYKS (SEQ ID NO:277).

113-115. (canceled)

116. A method of treating or effecting prophylaxis of a tau-related disease in a subject comprising administering an immunogen comprising a tau peptide of up to 20 contiguous amino acids of SEQ ID NO:1 to which antibody 9F5, 10C12, 2D11, 12C4, 17C12, or 14H3 specifically binds, wherein the peptide induces formation of antibodies specifically binding to tau in the subject.
